(19) United States
(12) Patent Application Publication
(10) Pub. No.: US 2025/0248998 A1
(43) Pub. Date: Aug. 7, 2025

(54) LRRK2 Inhibitors

(71) Applicant: Brenig Therapeutics, Inc., San Diego, CA (US)

(72) Inventors: Volodymyr Kysil, San Diego, CA (US); Vladislav Zenonovich Parchinsky, Moscow (RU); Alexei Pushechnikov, San Diego, CA (US); Alexandre Vasilievich Ivachtchenko, Hallandale Beach, FL (US); Nikolay Savchuk, San Diego, CA (US)

(21) Appl. No.: 18/854,031

(22) PCT Filed: Mar. 4, 2023
(86) PCT No.: PCT/US2023/063745

§ 371 Date: Oct. 3, 2024

Related U.S. Application Data
(60) Provisional application No. 63/327,058, filed on Apr. 4, 2022.

(51) Int. Cl. A61K 31/501; A61K 31/5377; A61K 31/55; A61K 31/551; A61K 31/553; A61K 31/554; C07D 401/14; C07D 403/04; C07D 403/14; C07D 413/14; C07D 417/14; C07D 487/10; C07D 491/052; C07D 491/107; C07D 495/10

(52) U.S. Cl. CPC A61K 31/501; A61K 31/5377; A61K 31/55; A61K 31/551; A61K 31/553; A61K 31/554; C07D 401/14; C07D 403/04; C07D 403/14; C07D 413/14; C07D 417/14; C07D 487/10; C07D 491/052; C07D 491/107; C07D 495/10

(57) ABSTRACT

The present invention is directed to the compounds of Formula (I)—inhibitors of LRRK2. The inhibitors described herein can be useful in the treatment of diseases or disorders associated with LRRK2, such as Parkinson disease (PD). In particular, the invention is concerned with compounds and pharmaceutical compositions inhibiting LRRK2, methods of treating diseases or disorders associated with LRRK2, and methods of synthesizing these compounds.

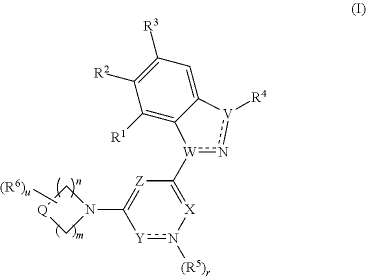

### CROSS-REFERENCE TO RELATED APPLICATIONS

This application claims priority to and the benefit of U.S. Provisional Patent Application Ser. No. 63/327,058 filed Apr. 4, 2022 entitled “LRRK2 Inhibitors,” the disclosure of which is incorporated herein by reference in its entirety for all purposes.

### FIELD OF INVENTION

The present invention is directed to inhibitors leucine-rich repeat kinase 2 (LRRK2). The inhibitors described herein can be useful in the treatment of diseases or disorders associated with LRRK2, such as Parkinson disease (PD). In particular, the invention is concerned with compounds and pharmaceutical compositions inhibiting LRRK2, methods of treating diseases or disorders associated with LRRK2, and methods of synthesizing these compounds.

### BACKGROUND

Parkinson's disease (PD), like many common age-related conditions, has been recognized to have a substantial genetic component. Parkinson's disease (PD) is a progressive neurodegenerative disorder that affects around 2% of individuals over 60 years old.

Multiple lines of evidence suggest that Leucine-rich repeat kinase 2 (LRRK2) is a crucial factor to understanding the etiology of PD. LRRK2 is a large, widely expressed, multi-domain and multifunctional protein. LRRK2 mutations are the major cause to inherited and sporadic PD. Recently, for sporadic PD, wild-type LRRK2 is also considered as a potential target. LRRK2 mutations, and particularly the most common mutation Gly2019Ser, are observed in patients with autosomal dominant PD and in those with apparent sporadic PD, who are clinically indistinguishable from those with idiopathic PD. The discoveries that pathogenic mutations in the LRRK2 gene increase LRRK2 kinase activity and that small-molecule LRRK2 kinase inhibitors can be neuroprotective in preclinical models of PD have placed LRRK2 at the centre of disease modification efforts in PD. Growing evidence highlights that LRRK2 controls multiple processes in brain immune cells, microglia and astrocytes, and suggests that deregulated LRRK2 activity in these cells, due to gene mutation, might be directly associated with pathological mechanisms underlying PD. Recent investigations also suggest that LRRK2 has a role in the pathogenesis of idiopathic PD and that LRRK2 therapies (LRRK2 mutated and wild) might, therefore, be beneficial in this common subtype of PD.

For the treatment of Parkinson's disease, comprehensive treatment should be taken, such as drug treatment, surgical therapy, rehabilitation therapy, psychological therapy and so on. Among them, drug treatment is the first choice and the main treatment. For example, deep brain stimulation (DBS) of the subthalamic nucleus (STN) is an established therapy for advanced PD and the STN-DBS outcomes were not influenced by the LRRK2 p.G2019S mutation. However, all the treatments just cannot stop the process of PD, needless to say to cure. The primary goals of all the therapies are to relieve the motor and non-motor symptoms of PD. Therefore, the requirement for new drugs to reverse, prevent, or slow down the progress of PD is one of the major goals for PD research.

### SUMMARY

A first aspect of the invention relates to compounds of Formula (I):

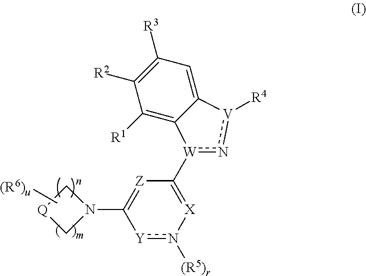

or a pharmaceutically acceptable salt, prodrug, stereoisomer, solvate, or tautomer thereof, wherein
V is selected from C and N;
W is selected from C and N;
and one from V and W is N;
bond V [symbol] N is select oni a single bond and a double bond;
bond W [symbol] N is selected from a single bond and a double bond;
provided that when V is N, the bond V [symbol] N is a single bond, the bond W [symbol] N is a double bond and W is C, and when V is C, the bond V [symbol] N is a double bond, the bond W [symbol] N is a single bond and W is N;
X is selected from CR⁷ and N;
Y is selected from C═O, CR⁸, and N;
provided that one from X and Y is N;
bond Y [symbol] N is selected from a single bond and a double bond;
r is an integer selected from 0 and 1;
provided that when Y is C═O, the bond Y [symbol] N is a single bond and r is 1 and when Y is CR⁸ or N, the bond Y [symbol] N is a double bond and r is 0;
when Y is C═O or N, Z is selected from CR⁹ and N;
when Y is CR⁸, Z is CR⁹;
Q is selected from CH₂, NR⁵, O, S, S(O), S(O)₂
R¹ is selected from H, halogen, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, NR¹⁰R¹¹, and S(O)₂R¹² wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R² is selected from H, halogen, C₁-C₆ alkyl, C₁-C₆ alkoxy, C₃-C₁₀ monocyclic cycloalkyl, C₃-C₁₀ cycloalkoxy, C₂-C₆ alkenyl, C₂-C₆ alkynyl, heterocycle, aryl, heteroaryl, C₁-C₆ alkyl-aryl, C₁-C₆ alkyl-heteroaryl, C₂-C₆ alkenyl-aryl, C₂-C₆ alkenyl-heteroaryl, C₂-C₆ alkynyl-aryl, C₂-C₆ alkynyl-heteroaryl, NR¹⁰R¹¹, and S(O)₂R¹² wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl; or
R¹ and R² together with the atoms to which they are attached and any intervening atoms, form a C₃-C₁₀ cycloalkyl, 5-14 membered heterocycle, aryl or heteroaryl wherein the cycloalkyl, heterocycle, aryl or heteroaryl is optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NHC₁-C₆ alkyl, and NR¹⁰R¹¹;
R³ is selected from H, halogen, C₁-C₆ alkyl, C₁-C₆ alkoxy, C₃-C₁₀ monocyclic cycloalkyl, C₃-C₁₀ cycloalkoxy, C₂-C₆ alkenyl, C₂-C₆ alkynyl, heterocycle, aryl, heteroaryl, C₁-C₆ alkyl-aryl, C₁-C₆ alkyl-heteroaryl, C₂-C₆ alkenyl-aryl, C₂-C₆ alkenyl-heteroaryl, C₂-C₆ alkynyl-aryl, C₂-C₆ alkynyl-heteroaryl, NR¹⁰R¹¹, and S(O)₂R¹² wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl; or
R² and R³ together with the atoms to which they are attached and any intervening atoms, form a C₃-C₁₀ cycloalkyl, 5-14 membered heterocycle, aryl or heteroaryl wherein the cycloalkyl, heterocycle, aryl or heteroaryl is optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NHC₁-C₆ alkyl, and NR¹⁰R¹¹;
R⁴ is selected from H, OH, CN, C₁-C₆ alkyl, C₃-C₁₀ cycloalkyl, C₁-C₆ alkyl-C₁-C₆ alkoxy, C₁-C₆ alkyl-NR¹⁰R¹¹, C(O)OC₁-C₆ alkyl, OC(O)C₁-C₆ alkyl wherein the alkyl, cycloalkyl or alkyl-alkoxy is optionally substituted with one or more substituents independently selected from halogen, oxo, OH, C₁-C₆ alkyl, C₁-C₆ alkoxy, NR¹⁰R¹¹, cycloalkyl, and heterocycle;
each R⁵ is independently selected from H, C₁-C₆ alkyl, C₃-C₁₀ cycloalkyl, C₁-C₆ alkyl-C₁-C₆ alkoxy, C₁-C₆ alkyl-NR¹⁰R¹¹, wherein the alkyl, cycloalkyl or alkyl-alkoxy is optionally substituted with one or more substituents independently selected from halogen, oxo, OH, C₁-C₆ alkyl, C₁-C₆ alkoxy, NR¹⁰R¹¹, cycloalkyl, and heterocycle;
each R⁶ is independently selected from halogen, OH, oxo, CN, CONR¹⁰R¹¹, NR¹⁰R¹¹, C₁-C₆ alkyl, C₁-C₆ alkoxy, C₁-C₆ alkyl-C₁-C₆ alkoxy, C₁-C₆ alkyl-C₃-C₈ cycloalkoxy, O—C₁-C₆ alkyl-C(O)NR¹⁰R¹¹, NR¹⁰C(O)R¹¹, —S(O)₂—C₁-C₆ alkyl, —C₁-C₆ alkanediyl-S(O)₂—C₁-C₆ alkyl, wherein the alkyl, or alkoxy is optionally substituted with one or more substituents independently selected from halogen, oxo, OH, C₁-C₆ alkyl, C₁-C₆ alkoxy, NR¹⁰R¹¹, cycloalkyl, and heterocycle;
or two R⁶ together with the atoms to which they are attached and any intervening atoms, form a 3-14 membered cycloalkyl, an aryl, a 3-14 membered heterocycle, or a 5-10 membered heteroaryl, wherein the cycloalkyl, aryl, heterocycle or heteroaryl is optionally substituted with one or more substituents independently selected from halogen, OH, CN, C₁-C₆ alkyl, C₁-C₆ alkoxy, C₁-C₆ hydroxyalkyl, C₁-C₆ alkyl-C₁-C₆ alkoxy, S(O)₂R¹²;
R⁷, R⁸ and R⁹ are each independently selected from H, C₁-C₆ alkyl, C₁-C₆ alkoxy, C₃-C₁₀ cycloalkyl, C₁-C₆ alkyl-C₁-C₆ alkoxy, C₁-C₆ alkyl-NR¹⁰R¹¹, wherein the alkyl, alkoxy, cycloalkyl or alkyl-alkoxy is optionally substituted with one or more substituents independently selected from halogen, oxo, OH, CN, C₁-C₆ alkyl, C₁-C₆ alkoxy, NR¹⁰R¹¹, S(O)₂R¹², cycloalkyl, and heterocycle;
R¹⁰ is selected from H, C₁-C₆ alkyl, C₃-C₁₀ cycloalkyl, S(O)₂R¹², wherein alkyl or cycloalkyl is optionally substituted with one or more substituents independently selected from halogen, oxo, OH, CN, C₁-C₆ alkyl, C₁-C₆ alkoxy;
R¹¹ is selected from H, C₁-C₆ alkyl, C₃-C₁₀ cycloalkyl, wherein alkyl or cycloalkyl is optionally substituted with one or more substituents independently selected from halogen, oxo, OH, CN, C₁-C₆ alkyl, C₁-C₆ alkoxy; or
R¹⁰ and R¹¹ together with the atoms to which they are attached and any intervening atoms, form a 5-14 membered heterocycle, wherein the heterocycle is optionally substituted with one or more substituents independently selected from halogen, OH, CN, C₁-C₆ alkyl, C₁-C₆ alkoxy;
R¹² is selected from C₁-C₆ alkyl, —NH₂, —NH(C₁-C₃ alkyl), or —N(C₁-C₃ alkyl)₂ wherein the alkyl is optionally substituted with one or more substituents independently selected from halogen, oxo, OH, CN, C₁-C₆ alkyl, C₁-C₆ alkoxy; wherein,
m is an integer selected from 1, 2, 3, 4;
n is an integer selected from 0, 1, 2, 3, 4;
u is an integer selected from 0, 1, 2, 3, 4, 5, 6;
aryl is cyclic, aromatic hydrocarbon groups that have 1 to 3 aromatic rings;
heterocyclyl is saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms selected from O, N, S, P, Se, or B;
heteroaryl is a monovalent monocyclic or a polycyclic aromatic radical of 5 to 24 ring atoms, containing one or more ring heteroatoms selected from N, O, S, P, or B, the remaining ring atoms being C.

Another aspect of the invention is directed to pharmaceutical compositions comprising a compound of Formula (I), or a pharmaceutically acceptable salt, hydrate, solvate, prodrug, stereoisomer, or tautomer thereof and a pharmaceutically acceptable carrier. The pharmaceutical acceptable carrier may further include an excipient, diluent, or surfactant.

Another aspect of the invention relates to a method of treating a disease or disorder associated with LRRK2. The method comprises administering to a patient in need of a treatment for diseases or disorders associated with LRRK2 an effective amount of a compound of Formula (I), or a pharmaceutically acceptable salt, hydrate, solvate, prodrug, stereoisomer, tautomer, or pharmaceutical composition thereof.

Another aspect of the invention is directed to a method of inhibiting of LRRK2. The method involves administering to a patient in need thereof an effective amount of a compound of Formula (I), or a pharmaceutically acceptable salt, hydrate, solvate, prodrug, stereoisomer, tautomer, or pharmaceutical composition thereof.

Another aspect of the present invention relates to compounds of Formula (I), or pharmaceutically acceptable salts, hydrates, solvates, prodrugs, stereoisomers, tautomers, or pharmaceutical compositions thereof, for use in the manufacture of a medicament for inhibiting LRRK2.

Another aspect of the present invention relates to the use of compounds of Formula (I), or pharmaceutically acceptable salts, hydrates, solvates, prodrugs, stereoisomers, tautomers, or pharmaceutical compositions thereof, in the treatment of diseases and disorders associated with LRRK2.

Another aspect of the present invention relates to compounds of Formula (I), or pharmaceutically acceptable salts, hydrates, solvates, prodrugs, stereoisomers, tautomers, or pharmaceutical compositions thereof, for use in the manufacture of a medicament for treating or preventing a disease or disorder disclosed herein.

Another aspect of the invention is directed to a method of treating or preventing a disease or disorder disclosed herein in a subject in need thereof. The method involves administering to a patient in need of the treatment an effective amount of a compound of Formula (I), or a pharmaceutically acceptable salt, hydrate, solvate, prodrug, stereoisomer, tautomer, or pharmaceutical composition thereof.

Another aspect of the present invention relates to the use of compounds of Formula (I), or pharmaceutically acceptable salts, hydrates, solvates, prodrugs, stereoisomers, tautomers, or pharmaceutical compositions thereof, in the treatment of a disease or disorder disclosed herein.

The present invention further provides methods of treating a disease or disorder associated with LRRK2, comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

The present invention provides inhibitors of LRRK2 that are therapeutic agents in the treatment of diseases and disorders.

The present invention further provides compounds and compositions with an improved efficacy and safety profile relative to known inhibitors of LRRK2. The present disclosure also provides agents with novel mechanisms of action toward LRRK2 in the treatment of various types of diseases.

The present invention further provides methods of treating a disease or disorder associated with LRRK2, comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

The present invention provides inhibitors of LRRK2 that are therapeutic agents in the treatment of diseases and disorders.

The present invention further provides methods of treating a disease, disorder, or condition selected from Parkinson Disease 8, Autosomal Dominant (PARK8); Hereditary Late-Onset Parkinson Disease (LOPD); Spinocerebellar Atrophy; Klippel-Feil Syndrome 1, Autosomal Dominant (KFS1); Autosomal Dominant Cerebellar Ataxia (SCA); Parkinson Disease, Late-Onset (PD); Parkinson Disease 2, Autosomal Recessive Juvenile (PARK2); Parkinsonism; Rem Sleep Behavior Disorder; Dementia, Lewy Body (DLB); Lrrk2 Parkinson Disease; Parkinson Disease 3, Autosomal Dominant (PARK3); Early-Onset Parkinson's Disease; Multiple System Atrophy 1 (MSA1); Essential Tremor; Movement Disease; Supranuclear Palsy, Progressive, 1 (PSNP1); Klippel-Feil Syndrome 1; Dementia; Parkinson Disease 10 (PARK10); Tremor; Frontotemporal Dementia (FTD); Postencephalitic Parkinson Disease; Vascular Parkinsonism; Aphasia; Parkinson Disease 1, Autosomal Dominant (PARK1); Athetosis; Klippel-Feil Syndrome (KFS); Kufor-Rakeb Syndrome (KRS); Leprosy 3 (LPRS3); Alzheimer Disease 8 (AD8); Crohn's Disease; Rheumatoid Arthritis (RA); Alzheimer Disease (AD); Color Agnosia; Gaucher Disease, Type I (GD1); Parkinson Disease 15, Autosomal Recessive Early-Onset (PARK15); Von Economo's Disease; Gerstmann-Straussler Disease (GSD); Amyotrophic Lateral Sclerosis-Parkinsonism/dementia Complex 1 (ALS-PDC1); Dystonia; Sphingolipidosis; Radial Nerve Lesion; Toxic Encephalopathy; Sleep Disorder; Pick Disease of Brain (PIDB); Ophthalmomyiasis; Gaucher's Disease (GD); Optic Atrophy 7 with or Without Auditory Neuropathy (OPA7); Ulnar Nerve Lesion; Inflammatory Bowel Disease 4 (IBD4); Inflammatory Bowel Disease; 3-Methylglutaconic Aciduria, Type Iii (MGCA3); Nervous System Disease; Amyotrophic Lateral Sclerosis 1 (ALS1); Mitochondrial Complex I Deficiency, Nuclear Type 1 (MC1DN1) comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

In some aspects, the present disclosure provides a compound obtainable by, or obtained by, a method for preparing compounds described herein (e.g., a method comprising one or more steps described in General Procedure A, B or C).

In some aspects, the present disclosure provides an intermediate as described herein, being suitable for use in a method for preparing a compound as described herein (e.g., the intermediate is selected from the intermediates described in Preparative part—P1-P44).

In some aspects, the present disclosure provides a method of preparing compounds of the present disclosure.

In some aspects, the present disclosure provides a method of preparing compounds of the present disclosure, comprising one or more steps described herein.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods, and examples are illustrative only and are not intended to be limiting. In the case of conflict between the chemical structures and names of the compounds disclosed herein, the chemical structures will control.

Other features and advantages of the disclosure will be apparent from the following detailed description and claims

### DETAILED DESCRIPTION

The present disclosure provides methods of treating, preventing, or ameliorating a disease or disorder associated with the inhibition LRRK2 by administering to a subject in need thereof a therapeutically effective amount of a compound as disclosed herein.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Definitions

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The term “optionally substituted” is understood to mean that a given chemical moiety (e.g., an alkyl group) can (but is not required to) be bonded other substituents (e.g., heteroatoms). For instance, an alkyl group that is optionally substituted can be a fully saturated alkyl chain (i.e., a pure hydrocarbon). Alternatively, the same optionally substituted alkyl group can have one or more substituents different from hydrogen. For instance, it can, at any point along the chain be bounded to a halogen atom, a hydroxyl group, or any other substituent described herein. Thus, the term “optionally substituted” means that a given chemical moiety has the potential to contain other functional groups but does not necessarily have any further functional groups. Suitable substituents used in the optional substitution of the described groups include, without limitation, halogen, oxo, —OH, —CN, —COOH, —CH₂CN, —O—(C₁-C₆) alkyl, (C₁-C₆) alkyl, (C₁-C₆) alkoxy, (C₁-C₆) haloalkyl, (C₁-C₆) haloalkoxy, —O—(C₂-C₆) alkenyl, —O—(C₂-C₆) alkynyl, (C₂-C₆) alkenyl, (C₂-C₆) alkynyl, —OH, —OP(O)(OH)₂, —OC(O)(C₁-C₆) alkyl, —C(O)(C₁-C₆) alkyl, —OC(O)O(C₁-C₆) alkyl, —NH₂, —NH((C₁-C₆) alkyl), —N((C₁-C₆) alkyl)₂, —NHC(O)(C₁-C₆) alkyl, —C(O)NH(C₁-C₆) alkyl, —S(O)₂(C₁-C₆) alkyl, —S(O)NH(C₁-C₆)alkyl, and —S(O)N((C₁-C₆)alkyl)₂. The substituents can themselves be optionally substituted. “Optionally substituted” as used herein also refers to substituted or unsubstituted whose meaning is described below.

As used herein, the term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

As used herein, the term “unsubstituted” means that the specified group bears no substituents.

Unless otherwise specifically defined, the term “aryl” refers to cyclic, aromatic hydrocarbon groups that have 1 to 3 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. Exemplary substituents include, but are not limited to, —H, -halogen, —O—(C₁-C₆)alkyl, (C₁-C₆)alkyl, —O—(C₂-C₆)alkenyl, —O—(C₂-C₆) alkynyl, (C₂-C₆)alkenyl, (C₂-C₆)alkynyl, —OH, —OP(O)(OH)₂, —OC(O)(C₁-C₆)alkyl, —C(O)(C₁-C₆) alkyl, —OC(O)O(C₁-C₆)alkyl, —NH₂, —NH((C₁-C₆)alkyl), —N((C₁-C₆)alkyl)₂, —S(O)₂—(C₁-C₆) alkyl, —S(O)NH(C₁-C₆)alkyl, and —S(O)N((C₁-C₆)alkyl)₂. The substituents can themselves be optionally substituted. Furthermore, when containing two fused rings the aryl groups herein defined may have one or more saturated or partially unsaturated ring fused with a fully unsaturated aromatic ring. Exemplary ring systems of these aryl groups include, but are not limited to, phenyl, biphenyl, naphthyl, anthracenyl, phenalenyl, phenanthrenyl, indanyl, indenyl, tetrahydronaphthalenyl, tetrahydrobenzoannulenyl, and the like.

Unless otherwise specifically defined, “heteroaryl” means a monovalent monocyclic or a polycyclic aromatic radical of 5 to 24 ring atoms, containing one or more ring heteroatoms selected from N, O, S, P, or B, the remaining ring atoms being C. A polycyclic aromatic radical includes two or more fused rings and may further include two or more spiro-fused rings, e.g., bicyclic, tricyclic, tetracyclic, and the like. Unless otherwise specifically defined, “fused” means two rings sharing two ring atoms. Unless otherwise specifically defined, “spiro-fused” means two rings sharing one ring atom. Heteroaryl as herein defined also means a bicyclic heteroaromatic group wherein the heteroatom is selected from N, O, S, P, or B. Heteroaryl as herein defined also means a tricyclic heteroaromatic group containing one or more ring heteroatoms selected from N, O, S, P, or B. Heteroaryl as herein defined also means a tetracyclic heteroaromatic group containing one or more ring heteroatoms selected from N, O, S, P, or B. The aromatic radical is optionally substituted independently with one or more substituents described herein. Examples include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, thiophen-2-yl, quinolyl, benzopyranyl, isothiazolyl, thiazolyl, thiadiazole, indazole, benzimidazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydro pyrrolo[1,2-a]pyrimidinyl, 3,4-dihydro-2H-1-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, benzooxazolyl, benzoisoxazolyl, furo[2,3-b]pyridinyl, benzothiophenyl, 1,5-naphthyridinyl, furo[3,2-b]pyridine, [1,2,4]triazolo[1,5-a]pyridinyl, benzo [1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo [1,5-b][1,2]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, 3H-indolyl, and derivatives thereof. Furthermore, when containing two or more fused rings, the heteroaryl groups defined herein may have one or more saturated or partially unsaturated ring fused with one or more fully unsaturated aromatic ring. In heteroaryl ring systems containing more than two fused rings, a saturated or partially unsaturated ring may further be fused with a saturated or partially unsaturated ring described herein. Furthermore, when containing three or more fused rings, the heteroaryl groups defined herein may have one or more saturated or partially unsaturated ring spiro-fused. Any saturated or partially unsaturated ring described herein is optionally substituted with one or more oxo. Exemplary ring systems of these heteroaryl groups include, for example, indolinyl, indolinonyl, dihydrobenzothiophenyl, dihydrobenzofuran, chromanyl, thiochromanyl, tetrahydroquinolinyl, dihydrobenzothiazine, 3,4-dihydro-1H-isoquinolinyl, 2,3-dihydrobenzofuranyl, benzofuranonyl, indolinyl, oxindolyl, indolyl, 1,6-dihydro-7H-pyrazolo[3,4-c]pyridin-7-onyl, 7,8-dihydro-6H-pyrido[3,2-b]pyrrolizinyl, 8H-pyrido[3,2-b]pyrrolizinyl, 1,5,6,7-tetrahydrocyclopenta[b]pyrazolo[4,3-e]pyridinyl, 7,8-dihydro-6H-pyrido[3,2-b]pyrrolizine, pyrazolo[1,5-a]pyrimidin-7(4H)-only, 3,4-dihydropyrazino[1,2-a]indol-1(2H)-onyl, benzo[c][1,2]oxaborol-1(3H)-olyl, 6,6a,7,8-tetrahydro-9H-pyrido[2,3-b]puyrrolo[1,2-d][1,4]oxazin-9-onyl, or 6a′,7′-dihydro-6′H,9′H-spiro[cyclopropane-1,8′-pyrido[2,3-b]pyrrolo[1,2-d][1,4]oxazin]-9′-onyl.

Halogen or “halo” refers to fluorine, chlorine, bromine, or iodine.

Alkyl refers to a straight or branched chain saturated hydrocarbon containing 1-12 carbon atoms. Examples of a (C₁-C₆) alkyl group include, but are not limited to, methyl, ethyl, propyl, butyl, pentyl, hexyl, isopropyl, isobutyl, sec-butyl, tert-butyl, isopentyl, neopentyl, and isohexyl.

“Alkoxy” refers to a straight or branched chain saturated hydrocarbon containing 1-12 carbon atoms containing a terminal “O” in the chain, i.e., —O(alkyl). Examples of alkoxy groups include without limitation, methoxy, ethoxy, propoxy, butoxy, t-butoxy, or pentoxy groups.

“Alkenyl” refers to a straight or branched chain unsaturated hydrocarbon containing 2-12 carbon atoms. The “alkenyl” group contains at least one double bond in the chain. The double bond of an alkenyl group can be unconjugated or conjugated to another unsaturated group. Examples of alkenyl groups include ethenyl, propenyl, n-butenyl, iso-butenyl, pentenyl, or hexenyl. An alkenyl group can be unsubstituted or substituted. Alkenyl, as herein defined, may be straight or branched.

“Alkynyl” refers to a straight or branched chain unsaturated hydrocarbon containing 2-12 carbon atoms. The “alkynyl” group contains at least one triple bond in the chain. Examples of alkenyl groups include ethynyl, propargyl, n-butynyl, iso-butynyl, pentynyl, or hexynyl. An alkynyl group can be unsubstituted or substituted.

The term “alkylene” or “alkylenyl” refers to a divalent alkyl radical. Any of the above mentioned monovalent alkyl groups may be an alkylene by abstraction of a second hydrogen atom from the alkyl. As herein defined, alkylene may also be a C₁-C₆ alkylene. An alkylene may further be a C₁-C₄ alkylene. Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

“Cycloalkyl” means mono or polycyclic saturated carbon rings containing 3-18 carbon atoms. Polycyclic cycloalkyl may be fused bicyclic cycloalkyl, bridged bicyclic cycloalkyl, or spiro-fused bicyclic cycloalkyl. A polycyclic cycloalkyl comprises at least one non-aromatic ring. Examples of cycloalkyl groups include, without limitations, cyclopropyl, cyclobutyl, cyclopentyl, cyclohexyl, cycloheptanyl, cyclooctanyl, norbornyl, norborenyl, 1,2,3,4-tetrahydronaphthyl, 2,3-dihydro-1H-indenyl, spiro[3.5]nonyl, spiro [5.5]undecyl, bicyclo[1.1.1]pentanyl, bicyclo[2.2.2]octanyl, or bicyclo[2.2.2]octenyl.

“Heterocyclyl”, “heterocycle” or “heterocycloalkyl” mono or polycyclic rings containing 3-24 atoms which include carbon and one or more heteroatoms selected from N, O, S, P, or B and wherein the rings are not aromatic. The heterocycloalkyl ring structure may be substituted by one or more substituents. The substituents can themselves be optionally substituted. Examples of heterocyclyl rings include, but are not limited to, oxetanyl, azetidinyl, tetrahydrofuranyl, tetrahydropyranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, oxazolidinonyl, and homotropanyl.

The term “aromatic” means a planar ring having 4n+2 electrons in a conjugated system. As used herein, “conjugated system” means a system of connected p-orbitals with delocalized electrons, and the system may include lone electron pairs.

The term “haloalkyl” as used herein refers to an alkyl group, as defined herein, which is substituted one or more halogen. Examples of haloalkyl groups include, but are not limited to, trifluoromethyl, difluoromethyl, pentafluoroethyl, trichloromethyl, etc.

The term “haloalkoxy” as used herein refers to an alkoxy group, as defined herein, which is substituted with one or more halogen. Examples of haloalkyl groups include, but are not limited to, trifluoromethoxy, difluoromethoxy, pentafluoroethoxy, trichloromethoxy, etc.

The term “cyano” as used herein means a substituent having a carbon atom joined to a nitrogen atom by a triple bond, i.e., C≡N.

“Spirocycloalkyl” or “spirocyclyl” means carbogenic bicyclic ring systems with both rings connected through a single atom. The ring can be different in size and nature, or identical in size and nature. Examples include spiropentane, spriohexane, spiroheptane, spirooctane, spirononane, or spirodecane. One or both of the rings in a spirocycle can be fused to another ring carbocyclic, heterocyclic, aromatic, or heteroaromatic ring. One or more of the carbon atoms in the spirocycle can be substituted with a heteroatom (e.g., O, N, S, or P). A (C₃-C₁₂) spirocycloalkyl is a spirocycle containing between 3 and 12 carbon atoms. One or more of the carbon atoms can be substituted with a heteroatom.

The term “spiroheterocycloalkyl”, “spiroheterocycle”, or “spiroheterocyclyl” is understood to mean a spirocycle wherein at least one of the rings is a heterocycle (e.g., at least one of the rings is furanyl, morpholinyl, or piperidinyl).

The term “solvate” refers to a complex of variable stoichiometry formed by a solute and solvent. Such solvents for the purpose of the disclosure may not interfere with the biological activity of the solute. Examples of suitable solvents include, but are not limited to, water, MeOH, EtOH, and AcOH. Solvates wherein water is the solvent molecule are typically referred to as hydrates. Hydrates include compositions containing stoichiometric amounts of water, as well as compositions containing variable amounts of water.

The term “isomer” refers to compounds that have the same composition and molecular weight but differ in physical and/or chemical properties. The structural difference may be in constitution (geometric isomers) or in the ability to rotate the plane of polarized light (stereoisomers). With regard to stereoisomers, the compounds of Formula (I) may have one or more asymmetric carbon atom and may occur as racemates, racemic mixtures and as individual enantiomers or diastereomers.

The present disclosure also contemplates isotopically-labelled compounds of Formula I (e.g., those labeled with ²H and ¹⁴C). Deuterated (i.e., ²H or D) and carbon-14 (i.e., ¹⁴C) isotopes are particularly preferred for their ease of preparation and detectability. Further, substitution with heavier isotopes such as deuterium may afford certain therapeutic advantages resulting from greater metabolic stability (e.g., increased in vivo half-life or reduced dosage requirements) and hence may be preferred in some circumstances. Isotopically labelled compounds of Formula I can generally be prepared by following procedures analogous to those disclosed in the Schemes and/or in the Examples herein below, by substituting an appropriate isotopically labelled reagent for a non-isotopically labelled reagent.

The disclosure also includes pharmaceutical compositions comprising a therapeutically effective amount of a disclosed compound and a pharmaceutically acceptable carrier. Representative “pharmaceutically acceptable salts” include, e.g., water-soluble and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate, pantothenate, phosphate/diphosphate, picrate, polygalacturonate, propionate, p-toluenesulfonate, salicylate, stearate, subacetate, succinate, sulfate, sulfosalicylate, tannate, tartrate, teoclate, tosylate, triethiodide, and valerate salts.

A “patient” or “subject” is a mammal, e.g., a human, mouse, rat, guinea pig, dog, cat, horse, cow, pig, or non-human primate, such as a monkey, chimpanzee, baboon, or rhesus.

An “effective amount” when used in connection with a compound is an amount effective for treating or preventing a disease in a subject as described herein.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treating” with regard to a subject, refers to improving at least one symptom of the subject's disorder. Treating includes curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

The term “prodrug,” as used in this disclosure, means a compound which is convertible in vivo by metabolic means (e.g., by hydrolysis) to a disclosed compound

The term “salt’ refers to pharmaceutically acceptable salts

The term “pharmaceutically acceptable salt” also refers to a salt of the compositions of the present disclosure having an acidic functional group, such as a carboxylic acid functional group, and a base.

“LRRK2 inhibitor” as used herein refer to compounds of Formula I and/or compositions comprising a compound of Formula I which inhibits LRRK2 kinase.

The amount of compound of composition described herein needed for achieving a therapeutic effect may be determined empirically in accordance with conventional procedures for the particular purpose. Generally, for administering therapeutic agents (e.g. compounds or compositions of Formula I (and/or additional agents) described herein) for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. For example, administration of therapeutic agents to a subject suffering from cancer provides a therapeutic benefit not only when the underlying condition is eradicated or ameliorated, but also when the subject reports a decrease in the severity or duration of the symptoms associated with the disease, e.g., a decrease in tumor burden, a decrease in circulating tumor cells, an increase in progression free survival. Therapeutic benefit also includes halting or slowing the progression of the underlying disease or disorder, regardless of whether improvement is realized.

### Compounds of the Present Disclosure

In one aspect, the present disclosure provides compounds of Formula (I) and salts, stereoisomers, solvates, prodrugs, isotopic derivatives, and tautomers thereof:

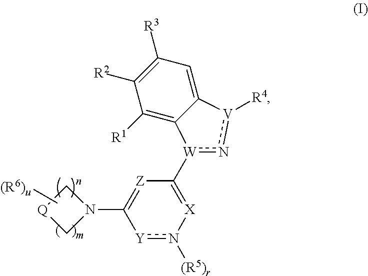

Wherein R¹, R², R³, R⁴, R⁵, R⁶, V, W, X, Y, Z, Q, m, n, r and u as described herein.

It is understood that, for a compound of Formula (I), R¹, R², R³, R⁴, R⁵, R⁶, V, W, X, Y, Z, Q, m, n, r and u can each be, where applicable, selected from the groups described herein, and any group described herein for any R¹, R², R³, R⁴, R⁵, R⁶, V, W, X, Y, Z, Q, m, n, r and u can be combined, where applicable, with any group described herein for one or more of the remainder of R¹, R², R³, R⁴, R⁵, R⁶, V, W, X, Y, Z, Q, m, n, r and u.

In some embodiments,
V is selected from C and N;
W is selected from C and N;
and one from V and W is N;
bond V [symbol] N is selected from a single bond and a double bond;
bond W [symbol] N is selected from a single bond and a double bond;
provided that when V is N, the bond V [symbol] N is a single bond, the bond W [symbol] N is a double bond and W is C, and when V is C, the bond V [symbol] N is a double bond, the bond W [symbol] N is a single bond and W is N;
X is selected from CR⁷ and N;
Y is selected from C═O, CR⁸, and N;
provided that one from X and Y is N;
bond Y [symbol] N is selected from a single bond and a double bond;
r is an integer selected from 0 and 1;
provided that when Y is C═O, the bond Y [symbol] N is a single bond and r is 1 and when Y is CR⁸ or N, the bond Y [symbol] N is a double bond and r is 0;
when Y is C═O or N, Z is selected from CR⁹ and N;
when Y is CR⁸, Z is CR⁹;
Q is selected from CH₂, NH, O, S, S(O), S(O)₂
R¹ is selected from H, halogen, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, NR¹⁰R¹¹, and S(O)₂R¹² wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R² is selected from H, halogen, C₁-C₆ alkyl, C₁-C₆ alkoxy, C₃-C₁₀ monocyclic cycloalkyl, C₃-C₁₀ cycloalkoxy, C₂-C₆ alkenyl, C₂-C₆ alkynyl, heterocycle, aryl, heteroaryl, C₁-C₆ alkyl-aryl, C₁-C₆ alkyl-heteroaryl, C₂-C₆ alkenyl-aryl, C₂-C₆ alkenyl-heteroaryl, C₂-C₆ alkynyl-aryl, C₂-C₆ alkynyl-heteroaryl, NR¹⁰R¹¹, and S(O)₂R¹² wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl; or
R¹ and R² together with the atoms to which they are attached and any intervening atoms, form a C₃-C₁₀ cycloalkyl, 5-14 membered heterocycle, aryl or heteroaryl wherein the cycloalkyl, heterocycle, aryl or heteroaryl is optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NHC₁-C₆ alkyl, and NR¹⁰R¹¹;
R³ is selected from H, halogen, C₁-C₆ alkyl, C₁-C₆ alkoxy, C₃-C₁₀ monocyclic cycloalkyl, C₃-C₁₀ cycloalkoxy, C₂-C₆ alkenyl, C₂-C₆ alkynyl, heterocycle, aryl, heteroaryl, C₁-C₆ alkyl-aryl, C₁-C₆ alkyl-heteroaryl, C₂-C₆ alkenyl-aryl, C₂-C₆ alkenyl-heteroaryl, C₂-C₆ alkynyl-aryl, C₂-C₆ alkynyl-heteroaryl, NR¹⁰R¹¹, and S(O)₂R¹² wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl; or
R² and R³ together with the atoms to which they are attached and any intervening atoms, form a C₃-C₁₀ cycloalkyl, 5-14 membered heterocycle, aryl or heteroaryl wherein the cycloalkyl, heterocycle, aryl or heteroaryl is optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NHC₁-C₆ alkyl, and NR¹⁰R¹¹;
R⁴ is selected from H, OH, CN, C₁-C₆ alkyl, C₃-C₁₀ cycloalkyl, C₁-C₆ alkyl-C₁-C₆ alkoxy, C₁-C₆ alkyl-NR¹⁰R¹¹, C(O)OC₁-C₆ alkyl, OC(O)C₁-C₆ alkyl wherein the alkyl, cycloalkyl or alkyl-alkoxy is optionally substituted with one or more substituents independently selected from halogen, oxo, OH, C₁-C₆ alkyl, C₁-C₆ alkoxy, NR¹⁰R¹¹, cycloalkyl, and heterocycle;
each R⁵ is independently selected from H, C₁-C₆ alkyl, C₃-C₁₀ cycloalkyl, C₁-C₆ alkyl-C₁-C₆ alkoxy, C₁-C₆ alkyl-NR¹⁰R¹¹, wherein the alkyl, cycloalkyl or alkyl-alkoxy is optionally substituted with one or more substituents independently selected from halogen, oxo, OH, C₁-C₆ alkyl, C₁-C₆ alkoxy, NR¹⁰R¹¹, cycloalkyl, and heterocycle;
each R⁶ is independently selected from halogen, OH, oxo, CN, CONR¹⁰R¹¹, NR¹⁰R¹¹, C₁-C₆ alkyl, C₁-C₆ alkoxy, C₁-C₆ alkyl-C₁-C₆ alkoxy, C₁-C₆ alkyl-C₃-C₈ cycloalkoxy, O—C₁-C₆ alkyl-C(O)NR¹⁰R¹¹, NR¹⁰C(O)R¹¹, —S(O)₂—C₁-C₆ alkyl, —C₁-C₆ alkanediyl-S(O)₂—C₁-C₆ alkyl, wherein the alkyl, or alkoxy is optionally substituted with one or more substituents independently selected from halogen, oxo, OH, C₁-C₆ alkyl, C₁-C₆ alkoxy, NR¹⁰R¹¹, cycloalkyl, and heterocycle;
or two R⁶ together with the atoms to which they are attached and any intervening atoms, form a 3-14 membered cycloalkyl, an aryl, a 3-14 membered heterocycle, or a 5-10 membered heteroaryl, wherein the cycloalkyl, aryl, heterocycle or heteroaryl is optionally substituted with one or more substituents independently selected from halogen, OH, CN, C₁-C₆ alkyl, C₁-C₆ alkoxy, C₁-C₆ hydroxyalkyl, C₁-C₆ alkyl-C₁-C₆ alkoxy, S(O)₂R¹²;
R⁷, R⁸ and R⁹ are each independently selected from H, C₁-C₆ alkyl, C₁-C₆ alkoxy, C₃-C₁₀ cycloalkyl, C₁-C₆ alkyl-C₁-C₆ alkoxy, C₁-C₆ alkyl-NR¹⁰R¹¹, wherein the alkyl, alkoxy, cycloalkyl or alkyl-alkoxy is optionally substituted with one or more substituents independently selected from halogen, oxo, OH, CN, C₁-C₆ alkyl, C₁-C₆ alkoxy, NR¹⁰R¹¹, S(O)₂R¹², cycloalkyl, and heterocycle;
R¹⁰ is selected from H, C₁-C₆ alkyl, C₃-C₁₀ cycloalkyl, S(O)₂R¹², wherein alkyl or cycloalkyl is optionally substituted with one or more substituents independently selected from halogen, oxo, OH, CN, C₁-C₆ alkyl, C₁-C₆ alkoxy;
R¹¹ is selected from H, C₁-C₆ alkyl, C₃-C₁₀ cycloalkyl, wherein alkyl or cycloalkyl is optionally substituted with one or more substituents independently selected from halogen, oxo, OH, CN, C₁-C₆ alkyl, C₁-C₆ alkoxy; or
R¹⁰ and R¹¹ together with the atoms to which they are attached and any intervening atoms, form a 5-14 membered heterocycle, wherein the heterocycle is optionally substituted with one or more substituents independently selected from halogen, OH, CN, C₁-C₆ alkyl, C₁-C₆ alkoxy;
R¹² is selected from C₁-C₆ alkyl, —NH₂, —NH(C₁-C₃ alkyl), or —N(C₁-C₃ alkyl)₂ wherein the alkyl is optionally substituted with one or more substituents independently selected from halogen, oxo, OH, CN, C₁-C₆ alkyl, C₁-C₆ alkoxy;
   wherein,
m is an integer selected from 1, 2, 3, 4;
n is an integer selected from 0, 1, 2, 3, 4;
u is an integer selected from 0, 1, 2, 3, 4, 5, 6.

In some embodiments, V is C or N. In some embodiments, V is C. In some embodiments, V is N.

In some embodiments, W is C or N. In some embodiments, W is C. In some embodiments, W is N.

In some embodiments, V is N, bond V [symbol] N is a single bond, bond W [symbol] N is a double bond, W is C.

In some embodiments, V is C, bond V [symbol] N is a double bond, bond W [symbol] N is a single bond, W is N.

In some embodiments, X is CR⁸ or N. In some embodiments, X is CR⁸. In some embodiments, X is N.

In some embodiments, X is CR⁸.

In some embodiments, X is N.

In some embodiments, Y is C═O, CR⁹, or N. In some embodiments, Y is C═O. In some embodiments, Y is CR⁹. In some embodiments, Y is N.

In some embodiments, Y is C═O.

In some embodiments, Y is CR⁹.

In some embodiments, Y is N.

In some embodiments, Z is CH or N. In some embodiments, Z is CH. In some embodiments, Z is N.

In some embodiments, Z is CH.

In some embodiments, when Y is N, Z is N as well.

In some embodiments, r is 0. In some embodiments, r is 1.

In some embodiments, r is 0.

In some embodiments, r is 1.

In some embodiments, when Y is C═O, the bond Y [symbol] N is a single bond.

In some embodiments, when Y is CR⁹ or N, the bond Y [symbol] N is a double bond.

In some embodiments, when Y is C═O, the bond Y [symbol] N is a single bond and r is 1.

In some embodiments, when Y is CR⁹ or N, the bond Y [symbol] N is a double bond and r is 0.

In some embodiments, R¹ is selected from H, halogen, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, NR¹⁰R¹¹, and S(O)₂R¹² wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl.

In some embodiments, R¹ is hydrogen.

In some embodiments, R¹ is halogen.

In some embodiments, R¹ is F. In some embodiments, R¹ is Cl. In some embodiments, R¹ is Br. In some embodiments, R¹ is I.

In some embodiments, R¹ is F.

In some embodiments, R¹ is C₁-C₆ alkyl.

In some embodiments, R¹ is —CH₃. In some embodiments, R¹ is —CH₂CH₃. In some embodiments, R¹ is —CH₂CH₂CH₃. In some embodiments, R¹ is —CH(CH₃)₂.

In some embodiments, R¹ is methyl.

In some embodiments, R¹ is —CH₂F. In some embodiments, R¹ is —CHF₂. In some embodiments, R¹ is —CF₃.

In some embodiments, R¹ is C₁-C₆ alkoxy.

In some embodiments, R¹ is —OCH₃. In some embodiments, R¹ is —OCH₂CH₃. In some embodiments, R¹ is —OCH₂CH₂CH₃. In some embodiments, R¹ is —OCH(CH₃)₂.

In some embodiments, R¹ is —OMe.

In some embodiments, R¹ is methoxy.

In some embodiments, R¹ is —OCH(CH₃)₂.

In some embodiments, R¹ is iso-propoxy.

In some embodiments, R¹ is —OC(CH₃)₃.

In some embodiments, R¹ is —OCF₃. In some embodiments, R¹ is —OCF₂CF₃.

In some embodiments, R¹ and R² together with the atoms to which they are attached and any intervening atoms, form a 5-14 membered heterocycle, wherein the heterocycle is optionally substituted with one or more substituents independently selected from halogen, OH, ═O, CN, NH₂, C₁-C₆ alkyl, C₁-C₆ alkoxy, C₁-C₆ alkyl-C₁-C₆ alkoxy, C₁-C₆ alkyl-NHC₁-C₆ alkyl, and NR¹⁰R¹¹.

In some embodiments, R¹ and R² together with the atoms to which they are attached and intervening atoms, form a 5 membered heterocycle, comprising one heteroatom selected from O, N, S.

In some embodiments, R¹ and R² together with the atoms to which they are attached and intervening atoms, form a 5 membered heterocycle, comprising one heteroatom —O.

In some embodiments, R¹ and R² together with the atoms to which they are attached and intervening atoms, form a 6 membered heterocycle, comprising one heteroatom selected from O, N, S.

In some embodiments, R¹ and R² together with the atoms to which they are attached and intervening atoms, form a 6 membered heterocycle, comprising one heteroatom —O.

In some embodiments, R¹ and R² together with the atoms to which they are attached and intervening atoms, form

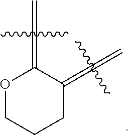 [SMILES: *=C1CCCOC1=*]

In some embodiments, R² is H. In some embodiments, R² is halogen. In some embodiments, R² is C₁-C₆ alkyl, optionally substituted with one or more substituents independently selected from halogen, OH, ═O, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl. In some embodiments, R² is C₁-C₆ alkoxy, optionally substituted with one or more substituents independently selected from halogen, OH, ═O, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl. In some embodiments, R² is C₃-C₁₀ monocyclic cycloalkyl, optionally substituted with one or more substituents independently selected from halogen, OH, ═O, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl. In some embodiments, R² is C₂-C₆ alkenyl, optionally substituted with one or more substituents independently selected from halogen, OH, ═O, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl. In some embodiments, R² is C₂-C₆ alkynyl, optionally substituted with one or more substituents independently selected from halogen, OH, ═O, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl. In some embodiments, R² is heterocycle, aryl, or heteroaryl, optionally substituted with one or more substituents independently selected from halogen, OH, ═O, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl. In some embodiments, R² is NR¹⁰R¹¹. In some embodiments, R² is S(O)₂R¹².

In some embodiments, R² is H.

In some embodiments, R² is C₁-C₆ alkyl.

In some embodiments, R² is methyl. In some embodiments, R² is ethyl. In some embodiments, R² is propyl. In some embodiments, R² is n-propyl. In some embodiments, R² is isopropyl. In some embodiments, R² is butyl. In some embodiments, R² is n-butyl. In some embodiments, R² is iso-butyl. In some embodiments, R² is sec-butyl. In some embodiments, R² is tert-butyl. In some embodiments, R² is pentyl. In some embodiments, R² is hexyl.

In some embodiments, R² is C₁-C₆ alkyl substituted with one or more halogen.

In some embodiments, R² is C₁-C₆ alkyl substituted with one or more F.

In some embodiments, R² is —CH₂F. In some embodiments, R² is —CHF₂. In some embodiments, R² is —CF₃. In some embodiments, R² is —CF₂CH₃. In some embodiments, R² is —CH₂CF₃. In some embodiments, R² is —CF₂CF₃. In some embodiments, R² is —CF₂CH(CH₃)₂.

In some embodiments, R² is

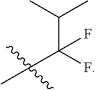 [SMILES: *C(F)(F)C(C)C]

In some embodiments, R² is C₁-C₆ alkyl substituted with one or more CN.

In some embodiments, R² is —CH₂CN. In some embodiments, R² is —CH(CH₃)CN. In some embodiments, R² is —C(CH₃)₂CN.

In some embodiments, R² is

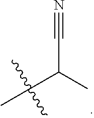 [SMILES: *C(C)C#N]

In some embodiments, R² is

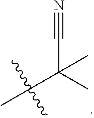 [SMILES: *C(C)(C)C#N]

In some embodiments, R² is C₁-C₆ alkoxy.

In some embodiments, R² is —OCH₃. In some embodiments, R² is —OCH₂CH₃. In some embodiments, R² is —OCH₂CH₂CH₃. In some embodiments, R² is —OCH(CH₃)₂. In some embodiments, R² is —OCH₂CH₂CH₂CH₃. In some embodiments, R² is —OCH₂CH(CH₃)₂. In some embodiments, R² is —OCH(CH₃)CH₂CH₃. In some embodiments, R² is —OC(CH₃)₃.

In some embodiments, R² is —OCH(CH₃)₂.

In some embodiments, R² is iso-propoxy.

In some embodiments, R² is —OC(CH₃)₃.

In some embodiments, R² is tert-butoxy.

In some embodiments, R² is C₁-C₆ alkoxy substituted with one or more halogen.

In some embodiments, R² is —OCH₂F. In some embodiments, R² is —OCHF₂. In some embodiments, R² is —OCF₃. In some embodiments, R² is —OCF₂CH₃. In some embodiments, R² is —OCH₂CF₃. In some embodiments, R² is —OCF₂CF₃.

In some embodiments, R² is

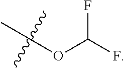 [SMILES: *OC(F)F]

In some embodiments, R² is

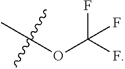 [SMILES: *OC(F)(F)F]

In some embodiments, R² is C₃-C₁₀ monocyclic cycloalkyl.

In some embodiments, R² is cyclopropyl. In some embodiments, R² is cyclobutyl. In some embodiments, R² is cyclopentyl. In some embodiments, R² is cyclohexyl. In some embodiments, R² is cycloheptyl.

In some embodiments, R² is C₃-C₁₀ monocyclic cycloalkyl substituted with one or more halogen.

In some embodiments, R² is C₃-C₁₀ monocyclic cycloalkyl substituted with one or more F.

In some embodiments, R² is C₃-C₁₀ monocyclic cycloalkyl substituted with one halogen.

In some embodiments, R² is C₃-C₁₀ monocyclic cycloalkyl substituted with one F.

In some embodiments, R² is cyclopropyl substituted with one F. In some embodiments, R² is cyclobutyl substituted with one F. In some embodiments, R² is cyclopentyl substituted with one F. In some embodiments, R² is cyclohexyl substituted with one F. In some embodiments, R² is cycloheptyl substituted with one F.

In some embodiments, R² is

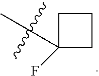 [SMILES: *C1(F)CCC1]

In some embodiments, R² is C₃-C₁₀ monocyclic cycloalkyl substituted with one or more CN.

In some embodiments, R² is C₃-C₁₀ monocyclic cycloalkyl substituted with one CN.

In some embodiments, R² is cyclopropyl substituted with one —CN. In some embodiments, R² is cyclobutyl substituted with one —CN. In some embodiments, R² is cyclopentyl substituted with one —CN. In some embodiments, R² is cyclohexyl substituted with one —CN. In some embodiments, R² is cycloheptyl substituted with one —CN.

In some embodiments, R² is

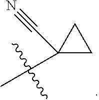 [SMILES: *C1(C#N)CC1]

In some embodiments, R² is

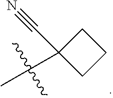 [SMILES: *C1(C#N)CCC1]

In some embodiments, R² is

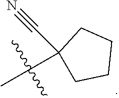 [SMILES: *C1(C#N)CCCC1]

In some embodiments, R² is

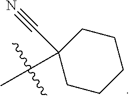 [SMILES: *C1(C#N)CCCCC1]

In some embodiments, R² is

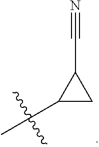 [SMILES: *C1CC1C#N]

In some embodiments, R² is

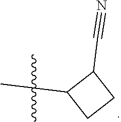 [SMILES: *C1CCC1C#N]

In some embodiments, R² is

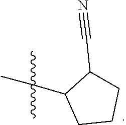 [SMILES: *C1CCCC1C#N]

In some embodiments, R² is

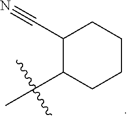 [SMILES: *C1CCCCC1C#N]

In some embodiments, R² is C₃-C₁₀ cycloalkoxy.

In some embodiments, R² is cyclopropoxy. In some embodiments, R² is cyclobutoxy. In some embodiments, R² is cyclopentoxy. In some embodiments, R² is cyclohexoxy. In some embodiments, R² is cycloheptoxy.

In some embodiments, R² is

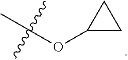 [SMILES: *OC1CC1]

In some embodiments, R² is

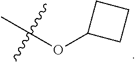 [SMILES: *OC1CCC1]

In some embodiments, R² is

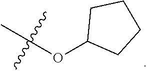 [SMILES: *OC1CCCC1]

In some embodiments, R² is

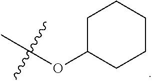 [SMILES: *OC1CCCCC1]

In some embodiments, R² is C₃-C₁₀ cycloalkoxy substituted with one or more C₁-C₆ alkyl.

In some embodiments, R² is C₃-C₁₀ cycloalkoxy substituted with one C₁-C₆ alkyl.

In some embodiments, R² is C₃-C₁₀ cycloalkoxy substituted with one methyl.

In some embodiments, R² is

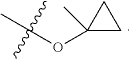 [SMILES: *OC1(C)CC1]

In some embodiments, R² is

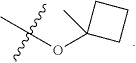 [SMILES: *OC1(C)CCC1]

In some embodiments, R² is

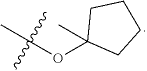 [SMILES: *OC1(C)CCCC1]

In some embodiments, R² is

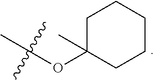 [SMILES: *OC1(C)CCCCC1]

In some embodiments, R² is

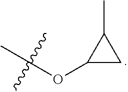 [SMILES: *OC1CC1C]

In some embodiments, R² is

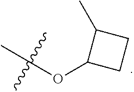 [SMILES: *OC1CCC1C]

In some embodiments, R² is

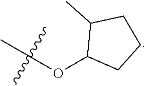 [SMILES: *OC1CCCC1C]

In some embodiments, R² is

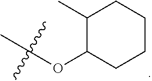 [SMILES: *OC1CCCCC1C]

In some embodiments, R² is

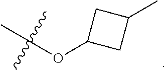 [SMILES: *OC1CC(C)C1]

In some embodiments, R² is

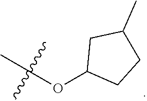 [SMILES: *OC1CCC(C)C1]

In some embodiments, R² is

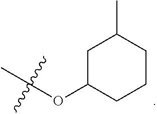 [SMILES: *OC1CCCC(C)C1]

In some embodiments, R² is

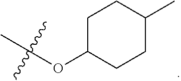 [SMILES: *OC1CCC(C)CC1]

In some embodiments, R² is heterocycle.

In some embodiments, R² is 3-10 membered heterocycle comprising 1-3 heteroatom is selected from O, N, S optionally substituted with one or more substituents independently selected from halogen, OH, ═O, NH₂, CN, C₁-C₆ alkyl, C₁-C₆ alkoxy, C₃-C₁₀ cycloalkyl, heterocycle, aryl, heteroaryl.

In some embodiments, R² is 5 membered heterocycle comprising S(O)₂ and substituted with 1-4 substituents selected from OH, ═O, NH₂, CN, C₁-C₆ alkyl.

In some embodiments, R² is

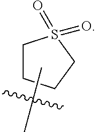

In some embodiments, R² is

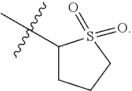 [SMILES: *C1CCCS1(=O)=O]

In some embodiments, R² is 5 membered heterocycle comprising N and substituted with 1-4 substituents selected from OH, ═O, NH₂, CN, C₁-C₆ alkyl.

In some embodiments, R² is

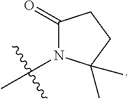 [SMILES: *N1C(=O)CCC1(C)C]

In some embodiments, R² is heteroaryl.

In some embodiments, R² is heteroaryl optionally substituted with one or more substituents independently selected from halogen, OH, ═O, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl.

In some embodiments, R² is heteroaryl comprising at least one heteroatom selected from N, O, S.

In some embodiments, R² is 5-membered heteroaryl.

In some embodiments, R² is 6-membered heteroaryl.

In some embodiments, R² is 5-membered heteroaryl comprising 0.

In some embodiments, R² is 5-membered heteroaryl comprising N.

In some embodiments, R² is 5-membered heteroaryl comprising S.

In some embodiments, R² is 5-membered heteroaryl comprising 0 and N.

In some embodiments, R² is 5-membered heteroaryl comprising two N.

In some embodiments, R² is 5-membered heteroaryl comprising S and N.

In some embodiments, R² is 6-membered heteroaryl comprising 0.

In some embodiments, R² is 6-membered heteroaryl comprising N.

In some embodiments, R² is 6-membered heteroaryl comprising S.

In some embodiments, R² is 6-membered heteroaryl comprising 0 and N.

In some embodiments, R² is 6-membered heteroaryl comprising two N.

In some embodiments, R² is 6-membered heteroaryl comprising S and N.

In some embodiments, R² is heteroaryl substituted with —CH₃.

In some embodiments, R² is heteroaryl substituted with halogen.

In some embodiments, R² is heteroaryl substituted with —CN.

In some embodiments, R² is

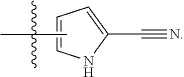

In some embodiments, R² is

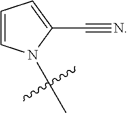 [SMILES: *n1cccc1C#N]

In some embodiments, R² is

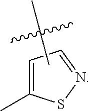

In some embodiments, R² is

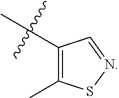 [SMILES: *c1cnsc1C]

In some embodiments, R² is —NR¹⁰R¹¹.

In some embodiments, R² is —NHS(O)₂R¹².

In some embodiments, R² is —NHS(O)₂R¹², wherein R¹² is C₁-C₆ alkyl optionally substituted with one or more halogen.

In some embodiments, R² is —NHS(O)₂R¹², wherein R¹² is C₁-C₆ alkyl optionally substituted with one or more F.

In some embodiments, R² is —NHS(O)₂CHF₂.

In some embodiments, R² is —S(O)₂R¹².

In some embodiments, R² is —S(O)₂R¹², wherein R¹² is C₁-C₆ alkyl optionally substituted with one or more halogen.

In some embodiments, R² is —S(O)₂CH₃.

In some embodiments, R² and R³ together with the atoms to which they are attached and any intervening atoms, form a 5-14 membered heterocycle, wherein the heterocycle is optionally substituted with one or more substituents independently selected from halogen, OH, ═O, CN, NH₂, C₁-C₆ alkyl, C₁-C₆ alkoxy, C₁-C₆ alkyl-C₁-C₆ alkoxy, C₁-C₆ alkyl-NHC₁-C₆ alkyl, and NR¹⁰R¹¹.

In some embodiments, R² and R³ together with the atoms to which they are attached and intervening atoms, form a 5 membered heterocycle, comprising one heteroatom selected from O, N, S.

In some embodiments, R² and R³ together with the atoms to which they are attached and intervening atoms, form a 5 membered heterocycle, comprising one heteroatom —O.

In some embodiments, R² and R³ together with the atoms to which they are attached and intervening atoms, form a 6 membered heterocycle, comprising one heteroatom selected from O, N, S.

In some embodiments, R² and R³ together with the atoms to which they are attached and intervening atoms, form a 6 membered heterocycle, comprising one heteroatom —O.

In some embodiments, R² and R³ together with the atoms to which they are attached and intervening atoms, form

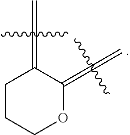 [SMILES: *=C1CCCOC1=*]

In some embodiments, R³ is selected from H, halogen, C₁-C₆ alkyl, C₁-C₆ alkoxy, C₃-C₁₀ monocyclic cycloalkyl, C₃-C₁₀ cycloalkoxy, C₂-C₆ alkenyl, C₂-C₆ alkynyl, heterocycle, aryl, heteroaryl, C₁-C₆ alkyl-aryl, C₁-C₆ alkyl-heteroaryl, C₂-C₆ alkenyl-aryl, C₂-C₆ alkenyl-heteroaryl, C₂-C₆ alkynyl-aryl, C₂-C₆ alkynyl-heteroaryl, NR¹⁰R¹¹, and S(O)₂R¹² wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl

In some embodiments, R³ is H.

In some embodiments, R³ is C₁-C₆ alkyl.

In some embodiments, R³ is methyl. In some embodiments, R³ is ethyl. In some embodiments, R³ is propyl. In some embodiments, R³ is n-propyl. In some embodiments, R³ is isopropyl. In some embodiments, R³ is butyl. In some embodiments, R³ is n-butyl. In some embodiments, R³ is isobutyl. In some embodiments, R³ is sec-butyl. In some embodiments, R³ is tert-butyl. In some embodiments, R³ is pentyl. In some embodiments, R³ is hexyl.

In some embodiments, R³ is —CH₃.

In some embodiments, R⁴ is selected from H, OH, CN, C₁-C₆ alkyl, C₃-C₁₀ cycloalkyl, C₁-C₆ alkyl-C₁-C₆ alkoxy, C₁-C₆ alkyl-NR¹⁰R¹¹, C(O)OC₁-C₆ alkyl, OC(O)C₁-C₆ alkyl wherein the alkyl, cycloalkyl or alkyl-alkoxy is optionally substituted with one or more substituents independently selected from halogen, oxo, OH, NH₂, C₁-C₆ alkyl, C₁-C₆ alkoxy, NR¹⁰R¹¹, cycloalkyl, and heterocycle;

In some embodiments, R⁴ is H.

In some embodiments, when V is C, R⁴ is halogen.

In some embodiments, R⁴ is F. In some embodiments, R⁴ is Cl. In some embodiments, R⁴ is Br. In some embodiments, R⁴ is I.

In some embodiments, when V is C, R⁴ is CN.

In some embodiments, when V is N, R⁴ is C(O)OC₁-C₆ alkyl.

In some embodiments, when V is N, R⁴ is C(O)O-tert-butyl.

In some embodiments, R⁵ is selected from H, C₁-C₆ alkyl, C₃-C₁₀ cycloalkyl, C₂-C₆ alkenyl, C₂-C₆ alkynyl, heterocycle, aryl, or heteroaryl wherein the alkyl, cycloalkyl, alkenyl, alkynyl, heterocycle, aryl, or heteroaryl is optionally substituted with one or more substituents independently selected from halogen, oxo, OH, NH₂, C₁-C₆ alkyl, C₁-C₆ alkoxy, NR¹⁰R¹¹, cycloalkyl, and heterocycle.

In some embodiments, R⁵ is H.

In some embodiments, R⁵ is C₁-C₆ alkyl.

In some embodiments, R⁵ is CH₃. In some embodiments, R⁵ is C₂H₅. In some embodiments, R⁵ is CH₂CH₂CH₃. In some embodiments, R⁵ is CH(CH₃)₂. In some embodiments, R⁵ is CH₂CH₂CH₂CH₃. In some embodiments, R⁵ is CH₂CH(CH₃)₂.

In some embodiments, R⁵ is methyl.

In some embodiments, R⁵ is ethyl.

In some embodiments, R⁵ is iso-propyl.

In some embodiments, R⁵ is iso-butyl.

In some embodiments, R⁵ is C₁-C₆ alkyl substituted with one or more halogen.

In some embodiments, R⁵ is C₁-C₆ alkyl substituted with one or more F.

In some embodiments, R⁵ is —CHF₂.

In some embodiments, R⁵ is —CF₃.

In some embodiments, R⁵ is C₃-C₁₀ cycloalkyl.

In some embodiments, R⁵ is cyclopropyl.

In some embodiments, R⁵ is cyclobutyl.

In some embodiments, R⁵ is cyclopentyl.

In some embodiments, Q is CH₂. In some embodiments Q is NR⁵. In some embodiments, Q is O. In some embodiments, Q is S. In some embodiments, Q is S(O). In some embodiments, Q is S(O)₂.

In some embodiments, Q is CH₂, optionally substituted with one or two R⁶.

In some embodiments, Q is CH₂.

In some embodiments, Q is CHR⁶.

In some embodiments, Q is C(R⁶)₂.

In some embodiments, Q is O.

In some embodiments, Q is NR⁵.

In some embodiments, Q is NH.

In some embodiments, Q is N(C₁-C₆ alkyl).

In some embodiments, Q is NCH₃.

In some embodiments, Q is NCH(CH₃)₂.

In some embodiments, Q is N(C₁-C₆ halogenalkyl).

In some embodiments, Q is NCH₂CF₃.

In some embodiments, Q is N(C₁-C₆ hydroxyalkyl).

In some embodiments, Q is

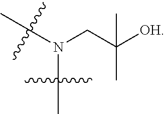 [SMILES: *N(*)CC(C)(C)O]

In some embodiments, Q is S(O)₂.

In some embodiments, m is 1. In some embodiments, m is 2. In some embodiments, m is 3. In some embodiments, m is 4.

In some embodiments, m is 1.

In some embodiments, m is 2.

In some embodiments, m is 3.

In some embodiments, m is 4.

In some embodiments, n is 0. In some embodiments, n is 1. In some embodiments, n is 2. In some embodiments, n is 3. In some embodiments, n is 4.

In some embodiments, n is 0.

In some embodiments, n is 1.

In some embodiments, n is 2.

In some embodiments, n is 3.

In some embodiments, n is 4.

In some embodiments, combination of m and n is selected from the table below:

| m | n |
|---|---|
| 1 | 0 |
| 1 | 1 |
| 1 | 2 |
| 1 | 3 |
| 1 | 4 |
| 2 | 0 |
| 2 | 1 |
| 2 | 2 |
| 2 | 3 |
| 2 | 4 |
| 3 | 0 |
| 3 | 1 |
| 3 | 2 |
| 3 | 3 |
| 3 | 4 |
| 4 | 0 |
| 4 | 1 |
| 4 | 2 |
| 4 | 3 |
| 4 | 4 |

In some embodiments, m is 1 and n is 2.

In some embodiments, m is 1, n is 2, and Q is CH₂.

In some embodiments, m is 1, n is 2, and Q is CHR⁶.

In some embodiments, m is 1, n is 2, and Q is C(R⁶)₂.

In some embodiments, m is 1, n is 2, and Q is C(CH₃)₂.

In some embodiments, m is 1, n is 2, and Q is

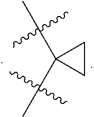 [SMILES: *C1(*)CC1]

In some embodiments, m is 1, n is 2, and Q is

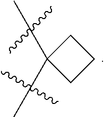 [SMILES: *C1(*)CCC1]

In some embodiments, m is 2 and n is 2.

In some embodiments, m is 2, n is 2, and Q is O.

In some embodiments, m is 2, n is 2, and Q is NR.

In some embodiments, m is 2, n is 2, and Q is S(O)₂.

In some embodiments, m is 2, n is 2, and Q is CH₂.

In some embodiments, m is 3 and n is 2.

In some embodiments, m is 3, n is 2, and Q is O.

In some embodiments, m is 3, n is 2, and Q is NR.

In some embodiments, m is 3, n is 2, and Q is NRH.

In some embodiments, m is 3, n is 2, and Q is NCH₃.

In some embodiments, m is 3, n is 2, and Q is NCH(CH₃)₂.

In some embodiments, m is 3, n is 2, and Q is NCH₂CF₃.

In some embodiments, m is 3, n is 2, and Q is

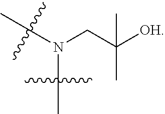 [SMILES: *N(*)CC(C)(C)O]

In some embodiments, m is 3, n is 2, and Q is CH₂.

In some embodiments, m is 3, n is 2, and Q is CHCH₃.

In some embodiments, m is 3, n is 2, and Q is C(CH₃)₂.

In some embodiments, m is 3, n is 2, and Q is CH(halogen).

In some embodiments, m is 3, n is 2, and Q is CHF.

In some embodiments, m is 3, n is 2, and Q is CH(OH).

In some embodiments, m is 3, n is 2, and Q is C(CH₃)OH.

In some embodiments, m is 3, n is 2, and Q is S(O)₂.

In some embodiments, u is 0. In some embodiments, u is 1. In some embodiments, u is 2. In some embodiments u is 3. In some embodiments u is 4. In some embodiments, u is 5. In some embodiments, u is 6.

In some embodiments, u is 0.

In some embodiments, u is 1.

In some embodiments, u is 2.

In some embodiments, u is 3.

In some embodiments, u is 4.

In some embodiments, each R⁶ is independently selected from halogen, OH, oxo, CN, CONR¹⁰R¹¹, NR¹⁰R¹¹, C₁-C₆ alkyl, C₁-C₆ alkoxy, C₁-C₆ alkyl-C₁-C₆ alkoxy, C₁-C₆ alkyl-C₃-C₈ cycloalkoxy, O—C₁-C₆ alkyl-C(O)NR¹⁰R¹¹, NR¹⁰R¹¹, —S(O)₂—C₁-C₆ alkyl, —C₁-C₆ alkanediyl-S(O)₂—C₁-C₆ alkyl, wherein the alkyl, or alkoxy is optionally substituted with one or more substituents independently selected from halogen, oxo, OH, C₁-C₆ alkyl, C₁-C₆ alkoxy, NR¹⁰R¹¹, cycloalkyl, and heterocycle.

In some embodiments, R⁶ is selected from halogen. In some embodiments, R⁶ is OH. In some embodiments, R⁶ is oxo. In some embodiments, R⁶ is CN. In some embodiments, R⁶ is CONR¹⁰R¹¹. In some embodiments, R⁶ is NR¹⁰R¹¹. In some embodiments, R⁶ is C₁-C₆ alkyl. In some embodiments, R⁶ is C₁-C₆ alkoxy. In some embodiments, R⁶ is O—C₁-C₆ alkyl-C(O)NR¹⁰R¹¹. In some embodiments, R⁶ is NR¹⁰C(O)R¹¹.

In some embodiments, R⁶ is halogen.

In some embodiments, R⁶ is F.

In some embodiments, R⁶ is OH.

In some embodiments, R⁶ is C₁-C₆ alkyl.

In some embodiments, R⁶ is —CH₃.

In some embodiments, R⁶ is C₁-C₆ halogenalkyl.

In some embodiments, R⁶ is —CF₃.

In some embodiments, R⁶ is —CH₂Cl.

In some embodiments, R⁶ is C₁-C₆ hydroxyalkyl.

In some embodiments, R⁶ is —CH₂OH.

In some embodiments, R⁶ is

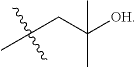 [SMILES: *CC(C)(C)O]

In some embodiments, R⁶ is C₁-C₆ alkyl-C₁-C₆ alkoxy.

In some embodiments, R⁶ is —CH₂—O—CH₃.

In some embodiments, R⁶ is —CH₂—O—CH₂CH₃.

In some embodiments, two R⁶ together with the atoms to which they are attached and any intervening atoms, form a 3-14 membered cycloalkyl, an aryl, a 3-14 membered heterocycle, or a 5-10 membered heteroaryl, wherein the cycloalkyl, aryl, heterocycle or heteroaryl is optionally substituted with one or more substituents independently selected from halogen, OH, CN, C₁-C₆ alkyl, C₁-C₆ alkoxy, C₁-C₆ hydroxyalkyl, C₁-C₆ alkyl-C₁-C₆ alkoxy, S(O)₂R¹².

In some embodiments, two geminal R⁶ together with the atoms to which they are attached and any intervening atoms, form a 3-14 membered cycloalkyl, or a 3-14 membered heterocycle, wherein the cycloalkyl, or heterocycle is optionally substituted with one or more substituents independently selected from halogen, OH, CN, C₁-C₆ alkyl, C₁-C₆ alkoxy, C₁-C₆ hydroxyalkyl, C₁-C₆ alkyl-C₁-C₆ alkoxy, S(O)₂R¹².

In some embodiments, two vicinal R⁶ together with the atoms to which they are attached and any intervening atoms, form a 3-14 membered cycloalkyl, an aryl, a 3-14 membered heterocycle, or a 5-10 membered heteroaryl, wherein the cycloalkyl, aryl, heterocycle or heteroaryl is optionally substituted with one or more substituents independently selected from halogen, OH, CN, C₁-C₆ alkyl, C₁-C₆ alkoxy, C₁-C₆ hydroxyalkyl, C₁-C₆ alkyl-C₁-C₆ alkoxy, S(O)₂R¹².

In some embodiments, two isolated R⁶ together with the atoms to which they are attached and any intervening atoms, form a 5-14 membered cycloalkyl, or 5-14 membered heterocycle, wherein the cycloalkyl, or heterocycle is optionally substituted with one or more substituents independently selected from halogen, OH, CN, C₁-C₆ alkyl, C₁-C₆ alkoxy, C₁-C₆ hydroxyalkyl, C₁-C₆ alkyl-C₁-C₆ alkoxy, S(O)₂R¹².

In some embodiments, two R⁶ together with the atoms to which they are attached and intervening atoms, form

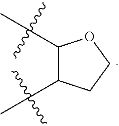 [SMILES: *C1CCOC1*]

In some embodiments, two R⁶ together with the atoms to which they are attached and intervening atoms, form

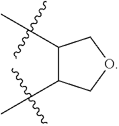 [SMILES: *C1COCC1*]

In some embodiments, two R⁶ together with the atom to which they are attached, form

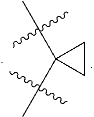 [SMILES: *C1(*)CC1]

In some embodiments, two R⁶ together with the atom to which they are attached, form

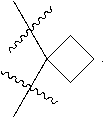 [SMILES: *C1(*)CCC1]

In some embodiments, two R⁶ together with the atom to which they are attached, form

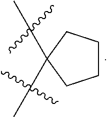 [SMILES: *C1(*)CCCC1]

In some embodiments, two R⁶ together with the atom to which they are attached, form

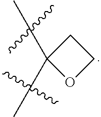 [SMILES: *C1(*)CCO1]

In some embodiments, two R⁶ together with the atom to which they are attached, form

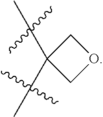 [SMILES: *C1(*)COC1]

In some embodiments, two R⁶ together with the atom to which they are attached, form

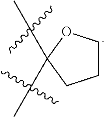 [SMILES: *C1(*)CCCO1]

In some embodiments, two R⁶ together with the atom to which they are attached, form

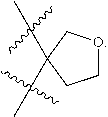 [SMILES: *C1(*)CCOC1]

In some embodiments, two R⁶ together with the atom to which they are attached, form

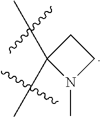 [SMILES: *C1(*)CCN1C]

In some embodiments, two R⁶ together with the atom to which they are attached, form

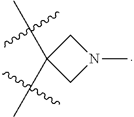 [SMILES: *C1(*)CN(C)C1]

In some embodiments, two R⁶ together with the atom to which they are attached, form

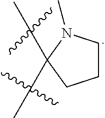 [SMILES: *C1(*)CCCN1C]

In some embodiments, two R⁶ together with the atom to which they are attached, form

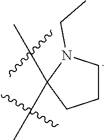 [SMILES: *C1(*)CCCN1CC]

In some embodiments, two R⁶ together with the atom to which they are attached, form

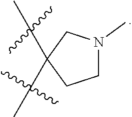 [SMILES: *C1(*)CCN(C)C1]

In some embodiments, two R⁶ together with the atom to which they are attached, form

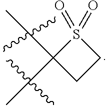 [SMILES: *C1(*)CCS1(=O)=O]

In some embodiments, two R⁶ together with the atom to which they are attached, form

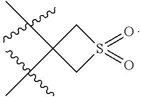 [SMILES: *C1(*)CS(=O)(=O)C1]

In some embodiments, two R⁶ together with the atom to which they are attached, form

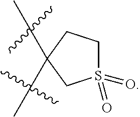 [SMILES: *C1(*)CCS(=O)(=O)C1]

In some embodiments, R⁷ is H. In some embodiments, R⁷ is C₁-C₆ alkyl optionally substituted with one or more substituents independently selected from halogen, oxo, OH, NH₂, C₁-C₆ alkyl, C₁-C₆ alkoxy, NR¹⁰R¹¹, cycloalkyl, and heterocycle. In some embodiments, R⁷ is C₃-C₁₀ cycloalkyl optionally substituted with one or more substituents independently selected from halogen, oxo, OH, NH₂, C₁-C₆ alkyl, C₁-C₆ alkoxy, NR¹⁰R¹¹, cycloalkyl, and heterocycle. In some embodiments, R⁷ is C₁-C₆ alkoxy optionally substituted with one or more substituents independently selected from halogen, oxo, OH, NH₂, C₁-C₆ alkyl, C₁-C₆ alkoxy, NR¹⁰R¹¹, cycloalkyl, and heterocycle. In some embodiments, R⁷ is C₂-C₆ alkenyl optionally substituted with one or more substituents independently selected from halogen, oxo, OH, NH₂, C₁-C₆ alkyl, C₁-C₆ alkoxy, NR¹⁰R¹¹, cycloalkyl, and heterocycle. In some embodiments, R⁷ is C₂-C₆ alkynyl optionally substituted with one or more substituents independently selected from halogen, oxo, OH, NH₂, C₁-C₆ alkyl, C₁-C₆ alkoxy, NR¹⁰R¹¹, cycloalkyl, and heterocycle. In some embodiments, R⁷ is heterocycle optionally substituted with one or more substituents independently selected from halogen, oxo, OH, NH₂, C₁-C₆ alkyl, C₁-C₆ alkoxy, NR¹⁰R¹¹, cycloalkyl, and heterocycle. In some embodiments, R⁷ is aryl optionally substituted with one or more substituents independently selected from halogen, oxo, OH, NH₂, C₁-C₆ alkyl, C₁-C₆ alkoxy, NR¹⁰R¹¹, cycloalkyl, and heterocycle. In some embodiments, R⁷ is heteroaryl optionally substituted with one or more substituents independently selected from halogen, oxo, OH, NH₂, C₁-C₆ alkyl, C₁-C₆ alkoxy, NR¹⁰R¹¹, cycloalkyl, and heterocycle.

In some embodiments, R⁷ is H.

In some embodiments, R⁸ is H. In some embodiments, R⁸ is C₁-C₆ alkyl optionally substituted with one or more substituents independently selected from halogen, oxo, OH, NH₂, C₁-C₆ alkyl, C₁-C₆ alkoxy, NR¹⁰R¹¹, cycloalkyl, and heterocycle. In some embodiments, R⁸ is C₃-C₁₀ cycloalkyl optionally substituted with one or more substituents independently selected from halogen, oxo, OH, NH₂, C₁-C₆ alkyl, C₁-C₆ alkoxy, NR¹⁰R¹¹, cycloalkyl, and heterocycle. In some embodiments, R⁸ is C₁-C₆ alkoxy optionally substituted with one or more substituents independently selected from halogen, oxo, OH, NH₂, C₁-C₆ alkyl, C₁-C₆ alkoxy, NR¹⁰R¹¹, cycloalkyl, and heterocycle. In some embodiments, R⁸ is C₂-C₆ alkenyl optionally substituted with one or more substituents independently selected from halogen, oxo, OH, NH₂, C₁-C₆ alkyl, C₁-C₆ alkoxy, NR¹⁰R¹¹, cycloalkyl, and heterocycle. In some embodiments, R⁸ is C₂-C₆ alkynyl optionally substituted with one or more substituents independently selected from halogen, oxo, OH, NH₂, C₁-C₆ alkyl, C₁-C₆ alkoxy, NR¹⁰R¹¹, cycloalkyl, and heterocycle. In some embodiments, R⁸ is heterocycle optionally substituted with one or more substituents independently selected from halogen, oxo, OH, NH₂, C₁-C₆ alkyl, C₁-C₆ alkoxy, NR¹⁰R¹¹, cycloalkyl, and heterocycle. In some embodiments, R⁸ is aryl optionally substituted with one or more substituents independently selected from halogen, oxo, OH, NH₂, C₁-C₆ alkyl, C₁-C₆ alkoxy, NR¹⁰R¹¹, cycloalkyl, and heterocycle. In some embodiments, R⁸ is heteroaryl optionally substituted with one or more substituents independently selected from halogen, oxo, OH, NH₂, C₁-C₆ alkyl, C₁-C₆ alkoxy, NR¹⁰R¹¹, cycloalkyl, and heterocycle.

In some embodiments, R⁸ is H.

In some embodiments, R⁸ is C₁-C₆ alkyl optionally substituted with one or more substituents independently selected from halogen, oxo, OH, NH₂, C₁-C₆ alkyl, C₁-C₆ alkoxy, NR¹⁰R¹¹, cycloalkyl, and heterocycle.

In some embodiments, R⁸ is C₁-C₆ alkyl.

In some embodiments, R⁸ is —CH₃.

In some embodiments, R⁹ is H. In some embodiments, R⁹ is C₁-C₆ alkyl optionally substituted with one or more substituents independently selected from halogen, oxo, OH, CN, C₁-C₆ alkyl, C₁-C₆ alkoxy, NR¹⁰R¹¹, cycloalkyl, and heterocycle. In some embodiments, R⁹ is C₃-C₁₀ cycloalkyl optionally substituted with one or more substituents independently selected from halogen, oxo, OH, CN, C₁-C₆ alkyl, C₁-C₆ alkoxy, NR¹⁰R¹¹, cycloalkyl, and heterocycle. In some embodiments, R⁹ is C₁-C₆ alkoxy optionally substituted with one or more substituents independently selected from halogen, oxo, OH, CN, C₁-C₆ alkyl, C₁-C₆ alkoxy, NR¹⁰R¹¹, cycloalkyl, and heterocycle. In some embodiments, R⁹ is C₂-C₆ alkenyl optionally substituted with one or more substituents independently selected from halogen, oxo, OH, CN, C₁-C₆ alkyl, C₁-C₆ alkoxy, NR¹⁰R¹¹, cycloalkyl, and heterocycle. In some embodiments, R⁹ is C₂-C₆ alkynyl optionally substituted with one or more substituents independently selected from halogen, oxo, OH, CN, C₁-C₆ alkyl, C₁-C₆ alkoxy, NR¹⁰R¹¹, cycloalkyl, and heterocycle. In some embodiments, R⁹ is heterocycle optionally substituted with one or more substituents independently selected from halogen, oxo, OH, CN, C₁-C₆ alkyl, C₁-C₆ alkoxy, NR¹⁰R¹¹, cycloalkyl, and heterocycle. In some embodiments, R⁹ is aryl optionally substituted with one or more substituents independently selected from halogen, oxo, OH, CN, C₁-C₆ alkyl, C₁-C₆ alkoxy, NR¹⁰R¹¹, cycloalkyl, and heterocycle. In some embodiments, R⁹ is heteroaryl optionally substituted with one or more substituents independently selected from halogen, oxo, OH, CN, C₁-C₆ alkyl, C₁-C₆ alkoxy, NR¹⁰R¹¹, cycloalkyl, and heterocycle.

In some embodiments, R⁹ is H.

In some embodiments, R⁹ is C₁-C₆ alkyl.

In some embodiments, R⁹ is —CH₃.

In some embodiments, R¹⁰ is H. In some embodiments, R¹⁰ is C₁-C₆ alkyl, optionally substituted with one or more substituents independently selected from halogen, oxo, OH, CN, C₁-C₆ alkyl, C₁-C₆ alkoxy. In some embodiments, R¹⁰ is C₃-C₁₀ cycloalkyl, optionally substituted with one or more substituents independently selected from halogen, oxo, OH, CN, C₁-C₆ alkyl, C₁-C₆ alkoxy. In some embodiments, R¹⁰ is S(O)₂R¹².

In some embodiments, R¹⁰ is H.

In some embodiments, R¹⁰ is C₁-C₆ alkyl.

In some embodiments, R¹⁰ is methyl.

In some embodiments, R¹⁰ is —S(O)₂R¹².

In some embodiments, R¹⁰ is —S(O)₂C₁-C₆ alkyl.

In some embodiments, R¹⁰ is —S(O)₂CH₃.

In some embodiments, R¹⁰ is —S(O)₂CHF₂.

In some embodiments, R¹¹ is H. In some embodiments, R¹¹ is C₁-C₆ alkyl, optionally substituted with one or more substituents independently selected from halogen, oxo, OH, CN, C₁-C₆ alkyl, C₁-C₆ alkoxy. In some embodiments, R¹¹ is C₃-C₁₀ cycloalkyl, optionally substituted with one or more substituents independently selected from halogen, oxo, OH, CN, C₁-C₆ alkyl, C₁-C₆ alkoxy.

In some embodiments, R¹¹ is H.

In some embodiments, R¹¹ is C₁-C₆ alkyl.

In some embodiments, R¹¹ is methyl.

In some embodiments, R¹² is C₁-C₆ alkyl, —NH₂, —NH(C₁-C₃ alkyl), or —N(C₁-C₃ alkyl)₂ wherein the alkyl is optionally substituted with one or more substituents independently selected from halogen, oxo, OH, CN, C₁-C₆ alkyl, C₁-C₆ alkoxy.

In some embodiments, R¹² is C₁-C₆ alkyl optionally substituted with one or more halogen.

In some embodiments, R¹² is —CHF₂.

In some embodiments, the compound is of Formula (I-I):

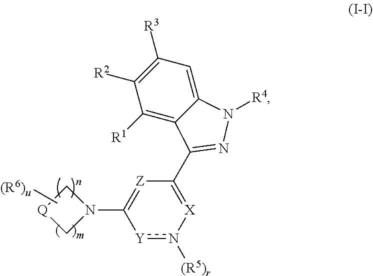

or a pharmaceutically acceptable salt, stereoisomer, solvate, prodrug, isotopic derivative, or tautomer thereof wherein all variables are as defined herein.

In some embodiments, the compound is of Formula (I-II):

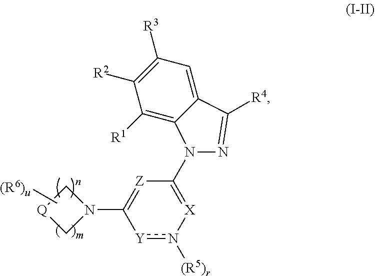

or a pharmaceutically acceptable salt, stereoisomer, solvate, prodrug, isotopic derivative, or tautomer thereof wherein all variables are as defined herein.

In some embodiments, the compound is of Formula (I-0):

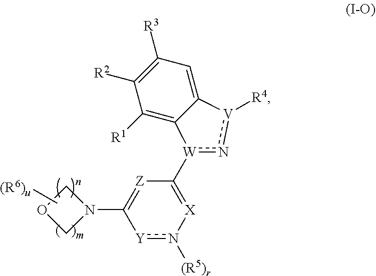

or a pharmaceutically acceptable salt, stereoisomer, solvate, prodrug, isotopic derivative, or tautomer thereof.

In some embodiments, the compound is of Formula (I-N):

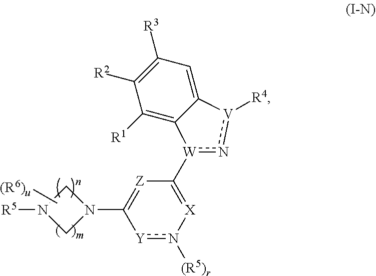

or a pharmaceutically acceptable salt, stereoisomer, solvate, prodrug, isotopic derivative, or tautomer thereof.

In some embodiments, the compound is of Formula (I-S):

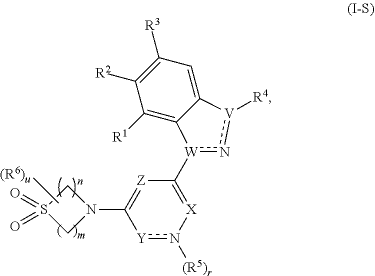

or a pharmaceutically acceptable salt, stereoisomer, solvate, prodrug, isotopic derivative, or tautomer thereof.

In some embodiments, the compound is of Formula (I-I):

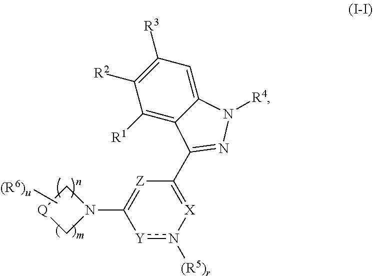

or a pharmaceutically acceptable salt, prodrug, stereoisomer, solvate, or tautomer thereof, wherein
X is selected from CR⁷ and N;
Y is selected from C═O, CR⁸, and N;
provided that on Y is N;
bond Y [symbol] N is selected from a single bond and a double bond;
r is an integer selected from 0 and 1;
provided that when Y is C═O, the bond Y [symbol] N is a single bond and r is 1 and when Y is CR⁸ or N, the bond Y [symbol] N is a double bond and r is 0;
when Y is C═O or N, Z is selected from CR⁹ and N;
when Y is CR⁸, Z is CR⁹;
Q is selected from CH₂, NR⁵, O, S, S(O), S(O)₂
R¹ is selected from H, halogen, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, NR¹⁰R¹¹, and S(O)₂R¹² wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R² is selected from H, halogen, C₁-C₆ alkyl, C₁-C₆ alkoxy, C₃-C₁₀ monocyclic cycloalkyl, C₃-C₁₀ cycloalkoxy, C₂-C₆ alkenyl, C₂-C₆ alkynyl, heterocycle, aryl, heteroaryl, C₁-C₆ alkyl-aryl, C₁-C₆ alkyl-heteroaryl, C₂-C₆ alkenyl-aryl, C₂-C₆ alkenyl-heteroaryl, C₂-C₆ alkynyl-aryl, C₂-C₆ alkynyl-heteroaryl, NR¹⁰R¹¹, and S(O)₂R¹² wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl; or
R¹ and R² together with the atoms to which they are attached and any intervening atoms, form a C₃-C₁₀ cycloalkyl, 5-14 membered heterocycle, aryl or heteroaryl wherein the cycloalkyl, heterocycle, aryl or heteroaryl is optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NHC₁-C₆ alkyl, and NR¹⁰R¹¹;
R³ is selected from H, halogen, C₁-C₆ alkyl, C₁-C₆ alkoxy, C₃-C₁₀ monocyclic cycloalkyl, C₃-C₁₀ cycloalkoxy, C₂-C₆ alkenyl, C₂-C₆ alkynyl, heterocycle, aryl, heteroaryl, C₁-C₆ alkyl-aryl, C₁-C₆ alkyl-heteroaryl, C₂-C₆ alkenyl-aryl, C₂-C₆ alkenyl-heteroaryl, C₂-C₆ alkynyl-aryl, C₂-C₆ alkynyl-heteroaryl, NR¹⁰R¹¹, and S(O)₂R¹² wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl; or
R² and R³ together with the atoms to which they are attached and any intervening atoms, form a C₃-C₁₀ cycloalkyl, 5-14 membered heterocycle, aryl or heteroaryl wherein the cycloalkyl, heterocycle, aryl or heteroaryl is optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NHC₁-C₆ alkyl, and NR¹⁰R¹¹;
R⁴ is selected from H, OH, CN, C₁-C₆ alkyl, C₃-C₁₀ cycloalkyl, C₁-C₆ alkyl-C₁-C₆ alkoxy, C₁-C₆ alkyl-NR¹⁰R¹¹, C(O)OC₁-C₆ alkyl, OC(O)C₁-C₆ alkyl wherein the alkyl, cycloalkyl or alkyl-alkoxy is optionally substituted with one or more substituents independently selected from halogen, oxo, OH, C₁-C₆ alkyl, C₁-C₆ alkoxy, NR¹⁰R¹¹, cycloalkyl, and heterocycle;
each R⁵ is independently selected from H, C₁-C₆ alkyl, C₃-C₁₀ cycloalkyl, C₁-C₆ alkyl-C₁-C₆ alkoxy, C₁-C₆ alkyl-NR¹⁰R¹¹, wherein the alkyl, cycloalkyl or alkyl-alkoxy is optionally substituted with one or more substituents independently selected from halogen, oxo, OH, C₁-C₆ alkyl, C₁-C₆ alkoxy, NR¹⁰R¹¹, cycloalkyl, and heterocycle;
each R⁶ is independently selected from halogen, OH, oxo, CN, CONR¹⁰R¹¹, NR¹⁰R¹¹, C₁-C₆ alkyl, C₁-C₆ alkoxy, C₁-C₆ alkyl-C₁-C₆ alkoxy, C₁-C₆ alkyl-C₃-C₈ cycloalkoxy, O—C₁-C₆ alkyl-C(O)NR¹⁰R¹¹, NR¹⁰C(O)R¹¹, —S(O)₂—C₁-C₆ alkyl, —C₁-C₆ alkanediyl-S(O)₂—C₁-C₆ alkyl, wherein the alkyl, or alkoxy is optionally substituted with one or more substituents independently selected from halogen, oxo, OH, C₁-C₆ alkyl, C₁-C₆ alkoxy, NR¹⁰R¹¹, cycloalkyl, and heterocycle;
or two R⁶ together with the atoms to which they are attached and any intervening atoms, form a 3-14 membered cycloalkyl, an aryl, a 3-14 membered heterocycle, or a 5-10 membered heteroaryl, wherein the cycloalkyl, aryl, heterocycle or heteroaryl is optionally substituted with one or more substituents independently selected from halogen, OH, CN, C₁-C₆ alkyl, C₁-C₆ alkoxy, C₁-C₆ hydroxyalkyl, C₁-C₆ alkyl-C₁-C₆ alkoxy, S(O)₂R¹²;
R⁷, R⁸ and R⁹ are each independently selected from H, C₁-C₆ alkyl, C₁-C₆ alkoxy, C₃-C₁₀ cycloalkyl, C₁-C₆ alkyl-C₁-C₆ alkoxy, C₁-C₆ alkyl-NR¹⁰R¹¹, wherein the alkyl, alkoxy, cycloalkyl or alkyl-alkoxy is optionally substituted with one or more substituents independently selected from halogen, oxo, OH, CN, C₁-C₆ alkyl, C₁-C₆ alkoxy, NR¹⁰R¹¹, S(O)₂R¹², cycloalkyl, and heterocycle;
R¹⁰ is selected from H, C₁-C₆ alkyl, C₃-C₁₀ cycloalkyl, S(O)₂R¹², wherein alkyl or cycloalkyl is optionally substituted with one or more substituents independently selected from halogen, oxo, OH, CN, C₁-C₆ alkyl, C₁-C₆ alkoxy;
R¹¹ is selected from H, C₁-C₆ alkyl, C₃-C₁₀ cycloalkyl, wherein alkyl or cycloalkyl is optionally substituted with one or more substituents independently selected from halogen, oxo, OH, CN, C₁-C₆ alkyl, C₁-C₆ alkoxy; or
R¹⁰ and R¹¹ together with the atoms to which they are attached and any intervening atoms, form a 5-14 membered heterocycle, wherein the heterocycle is optionally substituted with one or more substituents independently selected from halogen, OH, CN, C₁-C₆ alkyl, C₁-C₆ alkoxy;
R¹² is selected from C₁-C₆ alkyl, —NH₂, —NH(C₁-C₃ alkyl), or —N(C₁-C₃ alkyl)₂ wherein the alkyl is optionally substituted with one or more substituents independently selected from halogen, oxo, OH, CN, C₁-C₆ alkyl, C₁-C₆ alkoxy;
   wherein,
m is an integer selected from 1, 2, 3, 4;
n is an integer selected from 0, 1, 2, 3, 4;
u is an integer selected from 0, 1, 2, 3, 4, 5, 6;
aryl is cyclic, aromatic hydrocarbon groups that have 1 to 3 aromatic rings.

In some embodiments, the compound is of Formula (I-I-QN):

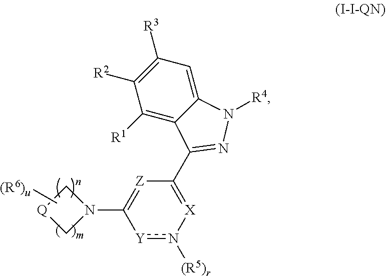

or a pharmaceutically acceptable salt, prodrug, stereoisomer, solvate, or tautomer thereof, wherein the

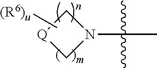

fragment is selected from:

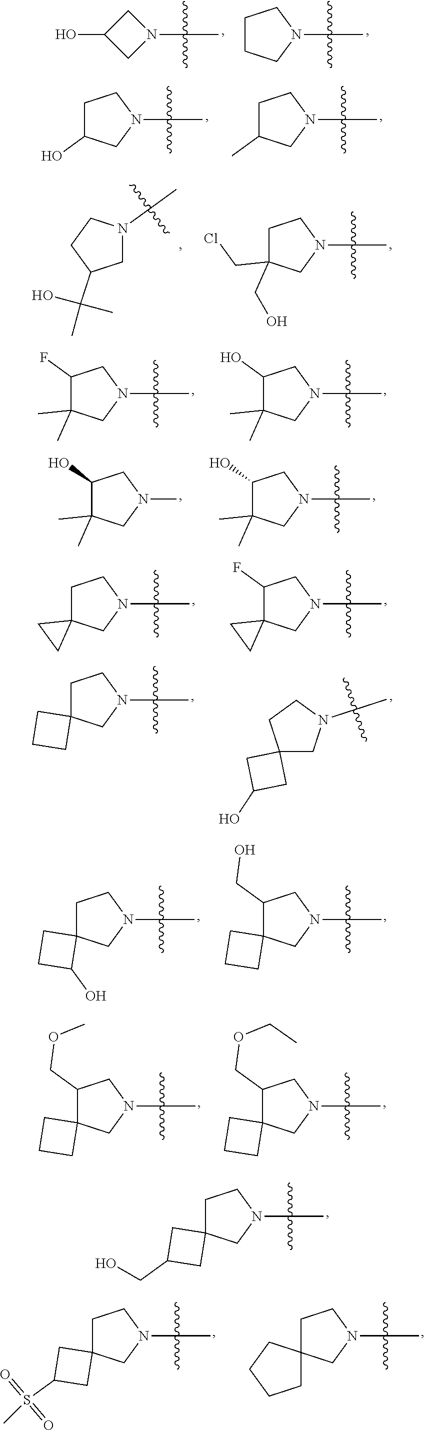

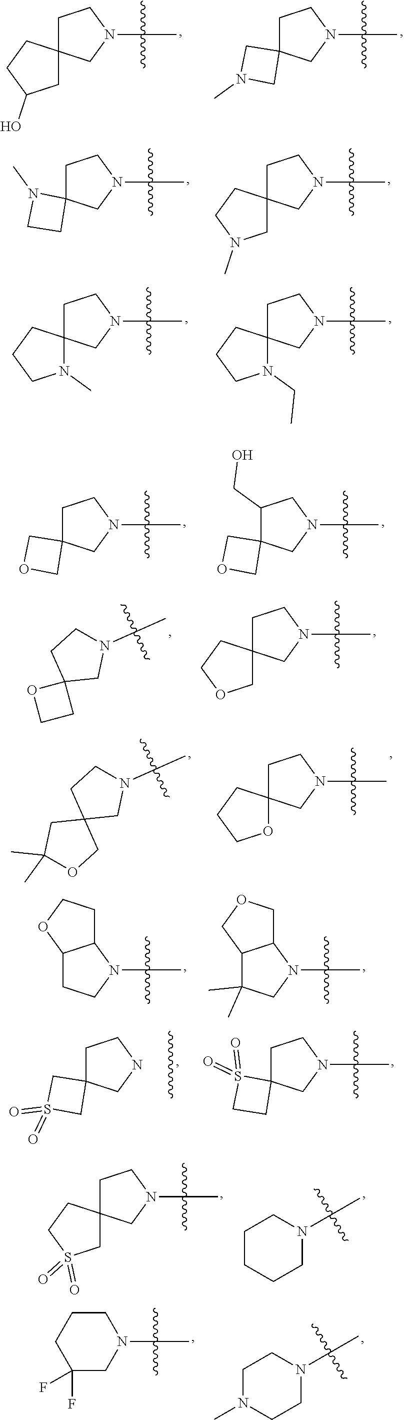

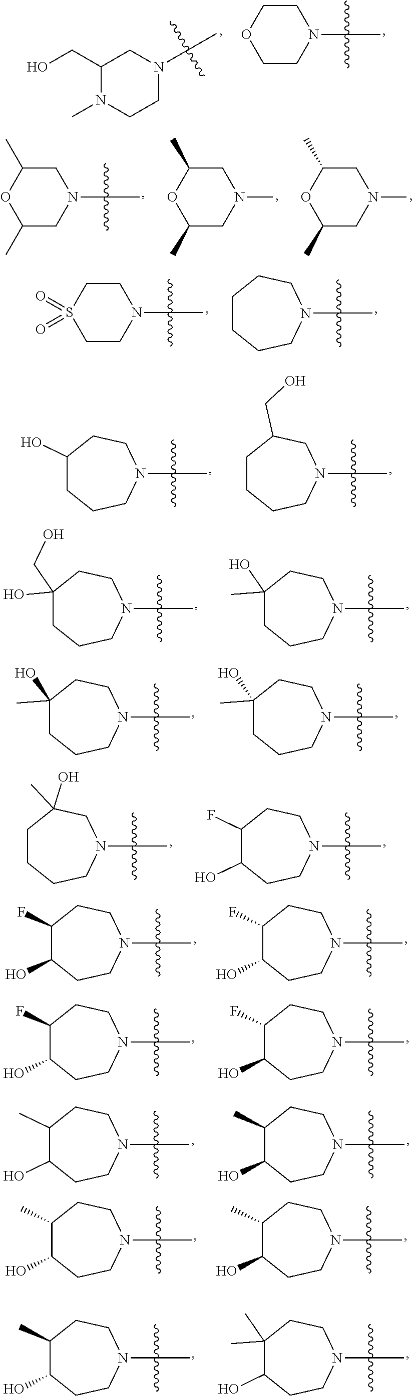

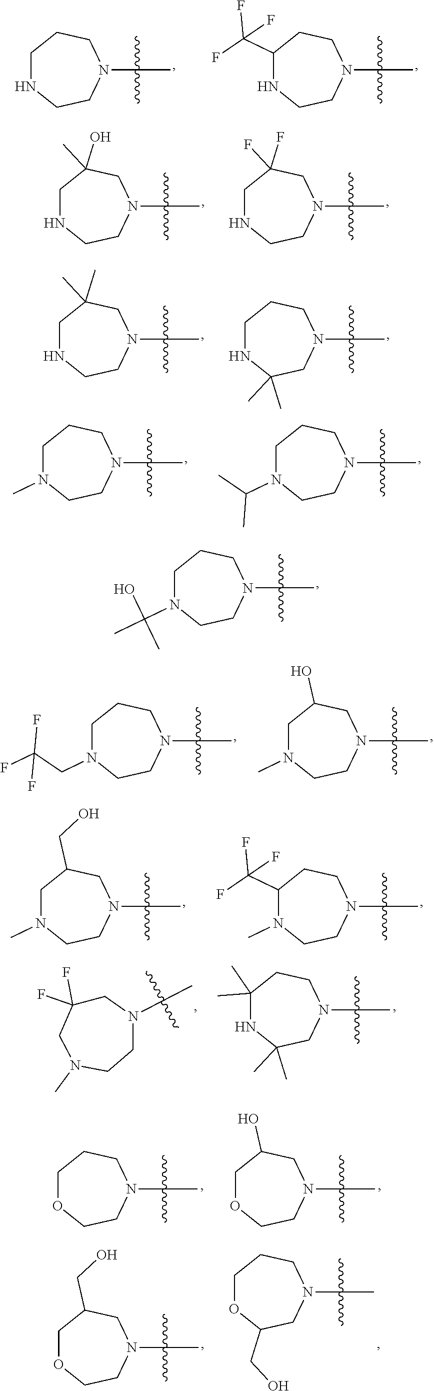

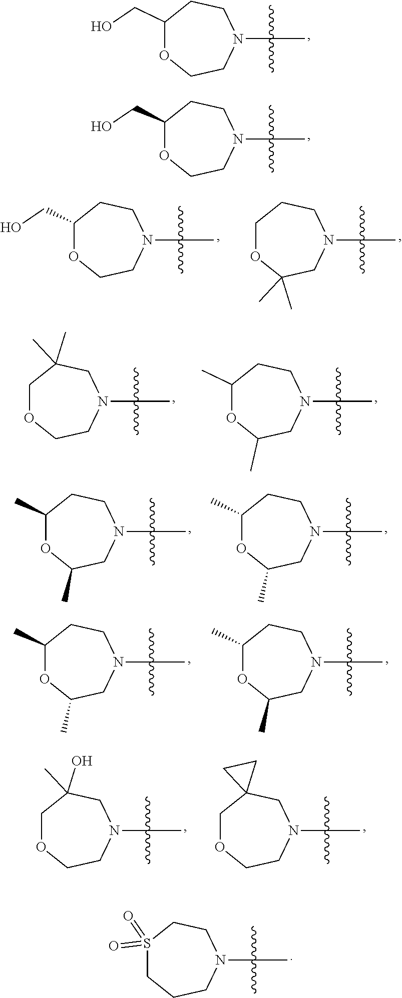

In some embodiments, the compound is of Formula (I-I-A):

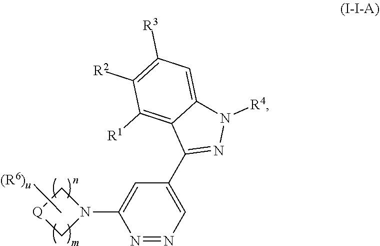

or a pharmaceutically acceptable salt, stereoisomer, solvate, prodrug, isotopic derivative, or tautomer thereof.

In some embodiments, the compound is of Formula (I-I-B):

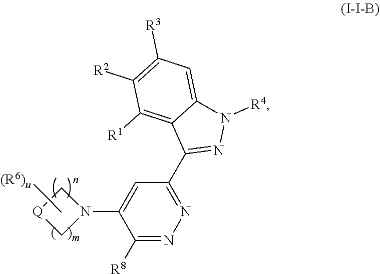

or a pharmaceutically acceptable salt, stereoisomer, solvate, prodrug, isotopic derivative, or tautomer thereof.

In some embodiments, the compound is of Formula (I-I-C):

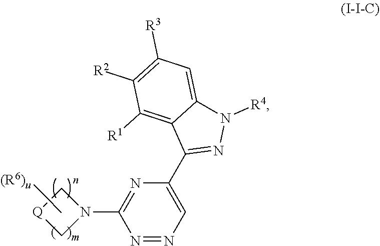

or a pharmaceutically acceptable salt, stereoisomer, solvate, prodrug, isotopic derivative, or tautomer thereof.

In some embodiments, the compound is of Formula (I-I-D):

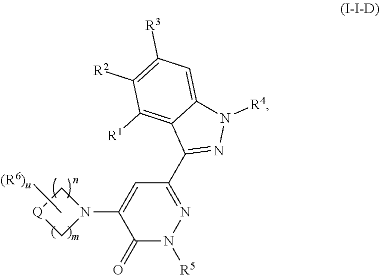

or a pharmaceutically acceptable salt, stereoisomer, solvate, prodrug, isotopic derivative, or tautomer thereof.

In some preferred embodiments, the compound is of Formula (I-I-D):

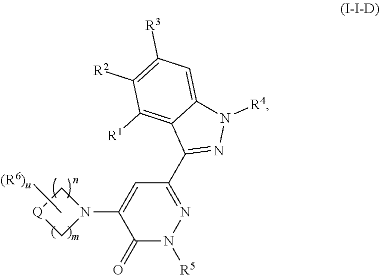

or a pharmaceutically acceptable salt, stereoisomer, solvate, prodrug, isotopic derivative, or tautomer thereof, wherein:
Q is selected from CH₂, NR⁵, O, S, S(O), S(O)₂
R¹ is selected from H, halogen, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, NR¹⁰R¹¹, and S(O)₂R¹² wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R² is selected from H, halogen, C₁-C₆ alkyl, C₁-C₆ alkoxy, C₃-C₁₀ monocyclic cycloalkyl, C₃-C₁₀ cycloalkoxy, C₂-C₆ alkenyl, C₂-C₆ alkynyl, heterocycle, aryl, heteroaryl, C₁-C₆ alkyl-aryl, C₁-C₆ alkyl-heteroaryl, C₂-C₆ alkenyl-aryl, C₂-C₆ alkenyl-heteroaryl, C₂-C₆ alkynyl-aryl, C₂-C₆ alkynyl-heteroaryl, NR¹⁰R¹¹, and S(O)₂R¹² wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl; or
R¹ and R² together with the atoms to which they are attached and any intervening atoms, form a C₃-C₁₀ cycloalkyl, 5-14 membered heterocycle, aryl or heteroaryl wherein the cycloalkyl, heterocycle, aryl or heteroaryl is optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NHC₁-C₆ alkyl, and NR¹⁰R¹¹;
R³ is selected from H, halogen, C₁-C₆ alkyl, C₁-C₆ alkoxy, C₃-C₁₀ monocyclic cycloalkyl, C₃-C₁₀ cycloalkoxy, C₂-C₆ alkenyl, C₂-C₆ alkynyl, heterocycle, aryl, heteroaryl, C₁-C₆ alkyl-aryl, C₁-C₆ alkyl-heteroaryl, C₂-C₆ alkenyl-aryl, C₂-C₆ alkenyl-heteroaryl, C₂-C₆ alkynyl-aryl, C₂-C₆ alkynyl-heteroaryl, NR¹⁰R¹¹, and S(O)₂R¹² wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl; or
R² and R³ together with the atoms to which they are attached and any intervening atoms, form a C₃-C₁₀ cycloalkyl, 5-14 membered heterocycle, aryl or heteroaryl wherein the cycloalkyl, heterocycle, aryl or heteroaryl is optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NHC₁-C₆ alkyl, and NR¹⁰R¹¹;
R⁴ is selected from H, OH, CN, C₁-C₆ alkyl, C₃-C₁₀ cycloalkyl, C₁-C₆ alkyl-C₁-C₆ alkoxy, C₁-C₆ alkyl-NR¹⁰R¹¹, C(O)OC₁-C₆ alkyl, OC(O)C₁-C₆ alkyl wherein the alkyl, cycloalkyl or alkyl-alkoxy is optionally substituted with one or more substituents independently selected from halogen, oxo, OH, C₁-C₆ alkyl, C₁-C₆ alkoxy, NR¹⁰R¹¹, cycloalkyl, and heterocycle;
each R⁵ is independently selected from H, C₁-C₆ alkyl, C₃-C₁₀ cycloalkyl, C₁-C₆ alkyl-C₁-C₆ alkoxy, C₁-C₆ alkyl-NR¹⁰R¹¹, wherein the alkyl, cycloalkyl or alkyl-alkoxy is optionally substituted with one or more substituents independently selected from halogen, oxo, OH, C₁-C₆ alkyl, C₁-C₆ alkoxy, NR¹⁰R¹¹, cycloalkyl, and heterocycle;
each R⁶ is independently selected from halogen, OH, oxo, CN, CONR¹⁰R¹¹, NR¹⁰R¹¹, C₁-C₆ alkyl, C₁-C₆ alkoxy, C₁-C₆ alkyl-C₁-C₆ alkoxy, C₁-C₆ alkyl-C₃-C₈ cycloalkoxy, O—C₁-C₆ alkyl-C(O)NR¹⁰R¹¹, NR¹⁰C(O)R¹¹, —S(O)₂—C₁-C₆ alkyl, —C₁-C₆ alkanediyl-S(O)₂—C₁-C₆ alkyl, wherein the alkyl, or alkoxy is optionally substituted with one or more substituents independently selected from halogen, oxo, OH, C₁-C₆ alkyl, C₁-C₆ alkoxy, NR¹⁰R¹¹, cycloalkyl, and heterocycle;
or two R⁶ together with the atoms to which they are attached and any intervening atoms, form a 3-14 membered cycloalkyl, an aryl, a 3-14 membered heterocycle, or a 5-10 membered heteroaryl, wherein the cycloalkyl, aryl, heterocycle or heteroaryl is optionally substituted with one or more substituents independently selected from halogen, OH, CN, C₁-C₆ alkyl, C₁-C₆ alkoxy, C₁-C₆ hydroxyalkyl, C₁-C₆ alkyl-C₁-C₆ alkoxy, S(O)₂R¹²;
R⁷, R⁸ and R⁹ are each independently selected from H, C₁-C₆ alkyl, C₁-C₆ alkoxy, C₃-C₁₀ cycloalkyl, C₁-C₆ alkyl-C₁-C₆ alkoxy, C₁-C₆ alkyl-NR¹⁰R¹¹, wherein the alkyl, alkoxy, cycloalkyl or alkyl-alkoxy is optionally substituted with one or more substituents independently selected from halogen, oxo, OH, CN, C₁-C₆ alkyl, C₁-C₆ alkoxy, NR¹⁰R¹¹, S(O)₂R¹², cycloalkyl, and heterocycle;
R¹⁰ is selected from H, C₁-C₆ alkyl, C₃-C₁₀ cycloalkyl, S(O)₂R¹², wherein alkyl or cycloalkyl is optionally substituted with one or more substituents independently selected from halogen, oxo, OH, CN, C₁-C₆ alkyl, C₁-C₆ alkoxy;
R¹¹ is selected from H, C₁-C₆ alkyl, C₃-C₁₀ cycloalkyl, wherein alkyl or cycloalkyl is optionally substituted with one or more substituents independently selected from halogen, oxo, OH, CN, C₁-C₆ alkyl, C₁-C₆ alkoxy; or
R¹⁰ and R¹¹ together with the atoms to which they are attached and any intervening atoms, form a 5-14 membered heterocycle, wherein the heterocycle is optionally substituted with one or more substituents independently selected from halogen, OH, CN, C₁-C₆ alkyl, C₁-C₆ alkoxy;
R¹² is selected from C₁-C₆ alkyl, —NH₂, —NH(C₁-C₃ alkyl), or —N(C₁-C₃ alkyl)₂ wherein the alkyl is optionally substituted with one or more substituents independently selected from halogen, oxo, OH, CN, C₁-C₆ alkyl, C₁-C₆ alkoxy; wherein,
m is an integer selected from 1, 2, 3, 4;
n is an integer selected from 0, 1, 2, 3, 4;
u is an integer selected from 0, 1, 2, 3, 4, 5, 6

In some preferred embodiments, the compound is of Formula (I-I-D-QN):

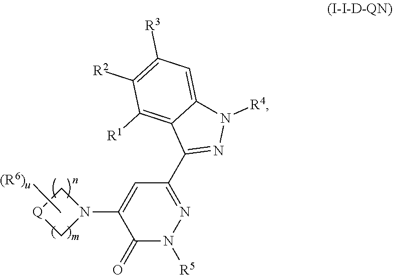

or a pharmaceutically acceptable salt, stereoisomer, solvate, prodrug, isotopic derivative, or tautomer thereof, wherein the fragment

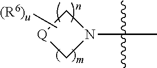

is selected from:

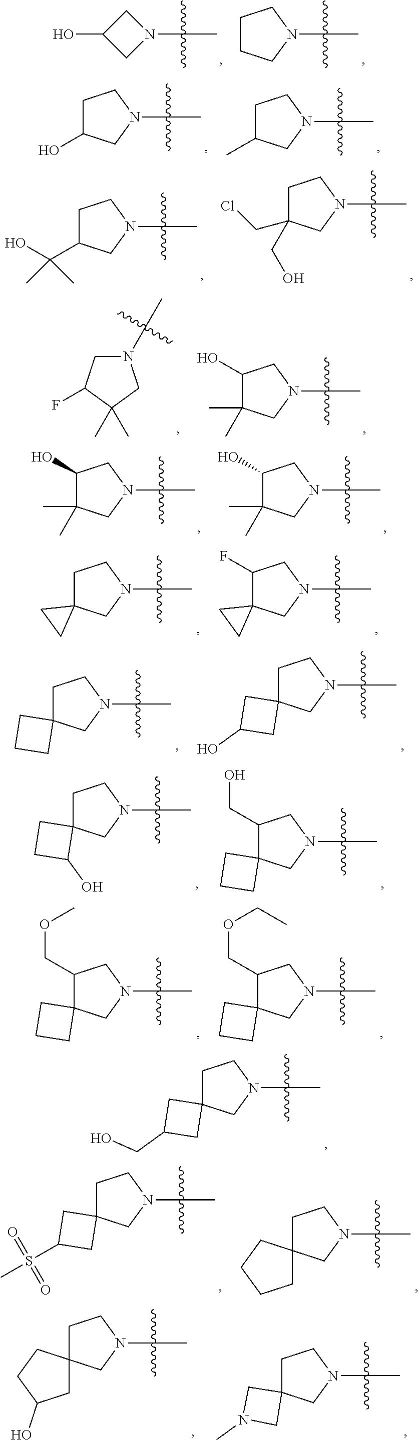

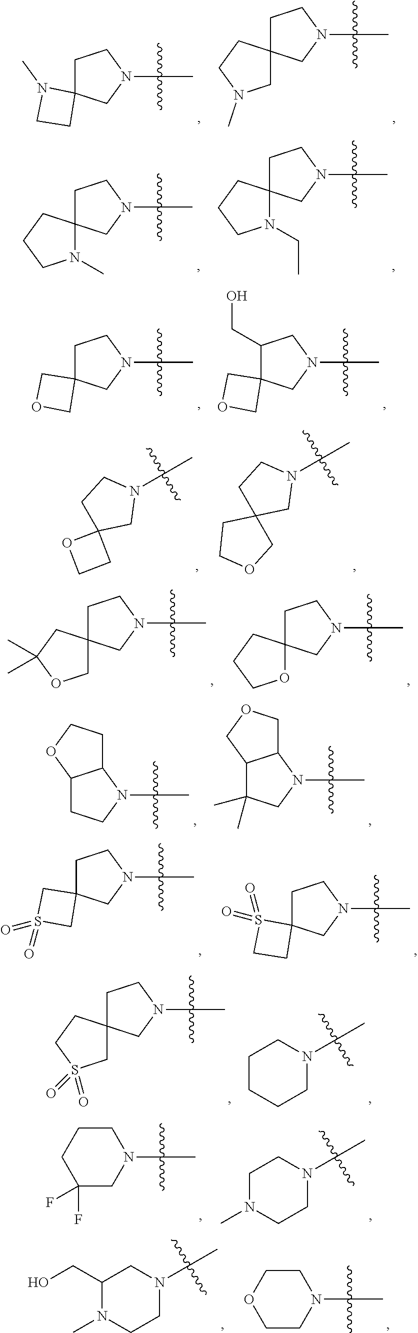

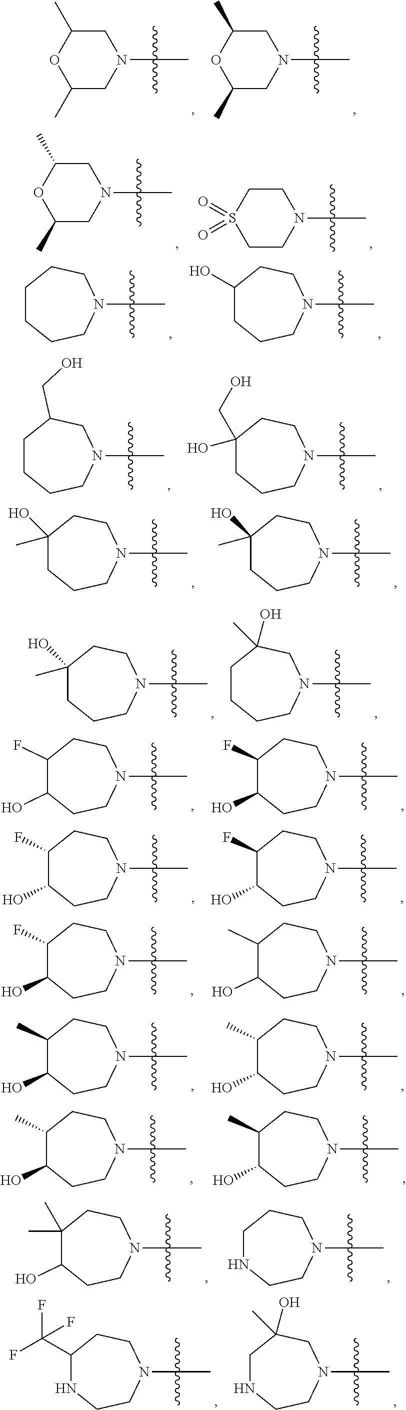

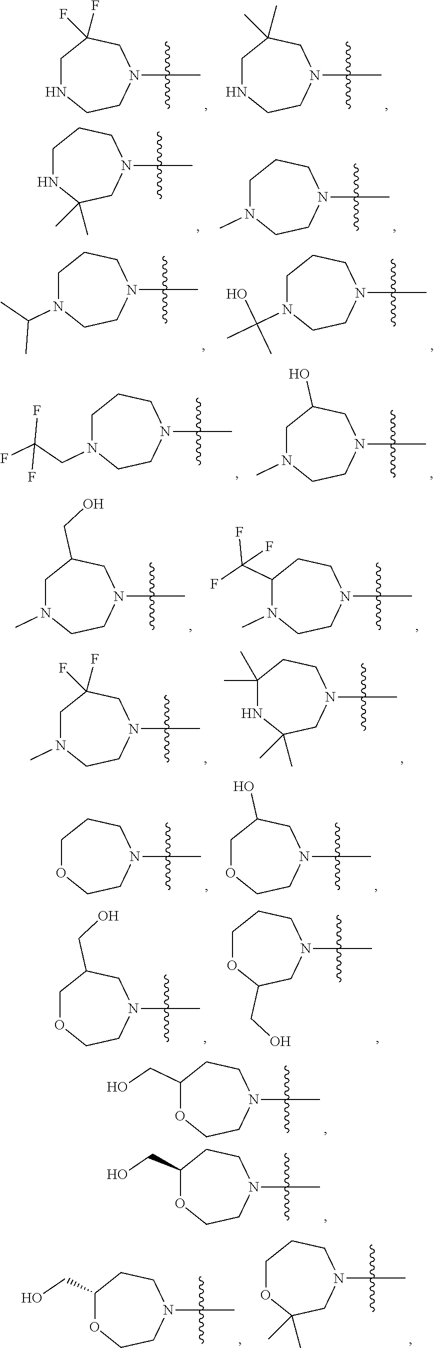

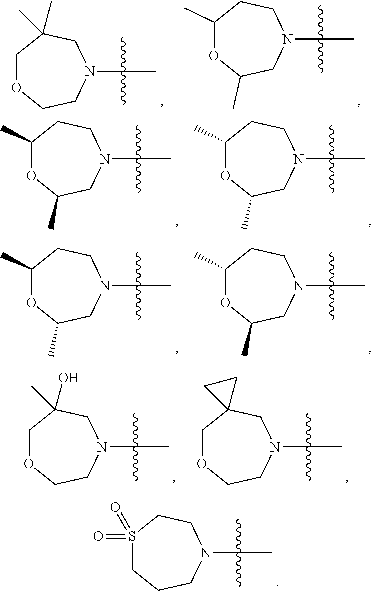

In some embodiments the compound is of Formula (I-II-A):

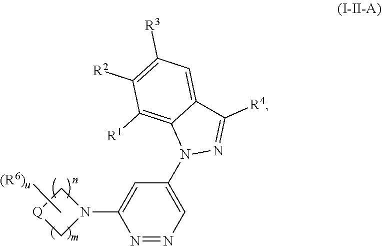

or a pharmaceutically acceptable salt, stereoisomer, solvate, prodrug, isotopic derivative, or tautomer thereof.

In some embodiments, the compound is of Formula (I-II-B):

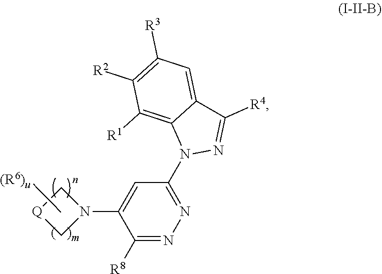

or a pharmaceutically acceptable salt, stereoisomer, solvate, prodrug, isotopic derivative, or tautomer thereof.

In some embodiments, the compound is of Formula (I-II-C):

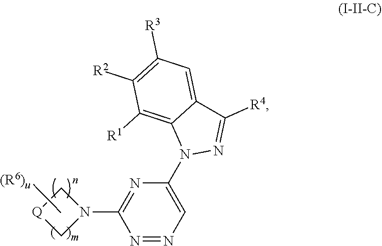

or a pharmaceutically acceptable salt, stereoisomer, solvate, prodrug, isotopic derivative, or tautomer thereof.

In some embodiments, the compound is of Formula (I-II-D):

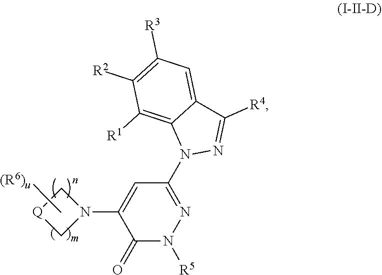

or a pharmaceutically acceptable salt, stereoisomer, solvate, prodrug, isotopic derivative, or tautomer thereof.

In some embodiments, the compound is of Formula (I-I-A-M):

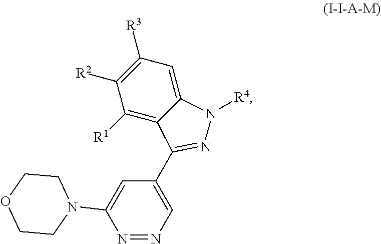

or a pharmaceutically acceptable salt, stereoisomer, solvate, prodrug, isotopic derivative, or tautomer thereof.

In some embodiments, the compound is of Formula (I-I-B-M):

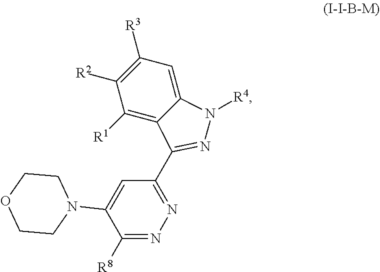

or a pharmaceutically acceptable salt, stereoisomer, solvate, prodrug, isotopic derivative, or tautomer thereof.

In some embodiments, the compound is of Formula (I-I-C-M):

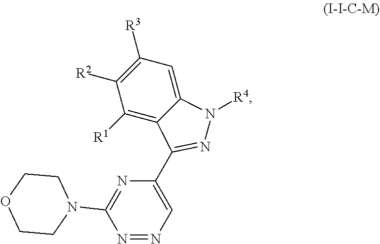

or a pharmaceutically acceptable salt, stereoisomer, solvate, prodrug, isotopic derivative, or tautomer thereof.

In some embodiments, the compound is of Formula (I-I-D-I):

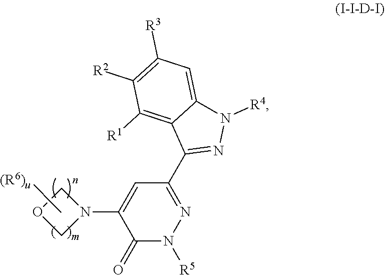

or a pharmaceutically acceptable salt, stereoisomer, solvate, prodrug, isotopic derivative, or tautomer thereof.

In some embodiments, the compound is of Formula (I-I-D-M):

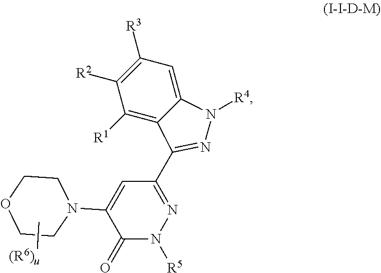

or a pharmaceutically acceptable salt, stereoisomer, solvate, prodrug, isotopic derivative, or tautomer thereof.

In some embodiments, the compound is of Formula (I-I-D-M-H):

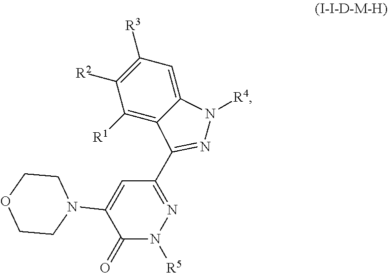

or a pharmaceutically acceptable salt, stereoisomer, solvate, prodrug, isotopic derivative, or tautomer thereof.

In some embodiments the compound is of Formula (I-I-D-M-A):

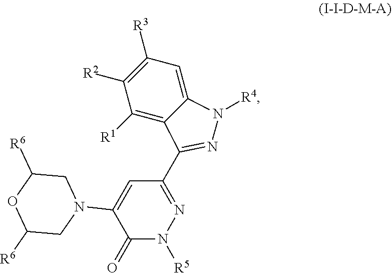

or a pharmaceutically acceptable salt, stereoisomer, solvate, prodrug, isotopic derivative, or tautomer thereof.

In some embodiments, the compound is of Formula (I-I-D-M-1):

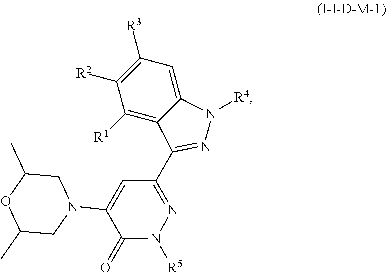

or a pharmaceutically acceptable salt, stereoisomer, solvate, prodrug, isotopic derivative, or tautomer thereof.

In some embodiments, the compound is of Formula (I-I-D-M-1*):

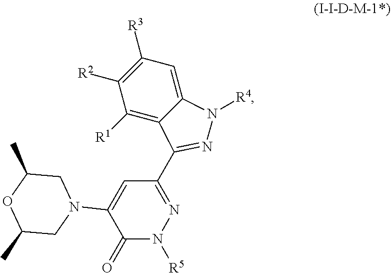

or a pharmaceutically acceptable salt, stereoisomer, solvate, prodrug, isotopic derivative, or tautomer thereof.

In some embodiments, the compound is of Formula (I-I-D-M-1**):

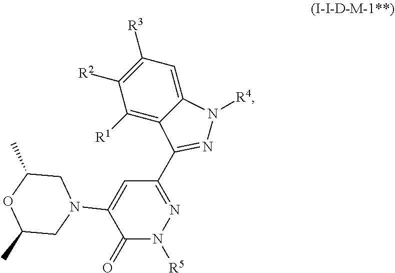

or a pharmaceutically acceptable salt, stereoisomer, solvate, prodrug, isotopic derivative, or tautomer thereof.

In some embodiments, the compound is of Formula (I-I-D-O):

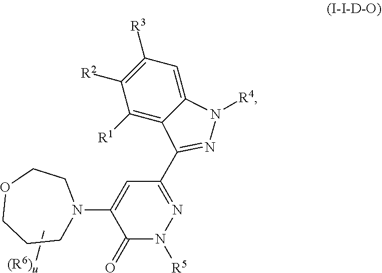

or a pharmaceutically acceptable salt, stereoisomer, solvate, prodrug, isotopic derivative, or tautomer thereof.

In some embodiments, the compound is of Formula (I-I-D-O—H):

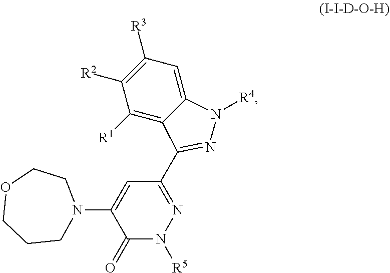

or a pharmaceutically acceptable salt, stereoisomer, solvate, prodrug, isotopic derivative, or tautomer thereof.

In some embodiments, the compound is of Formula (I-I-D-O-A):

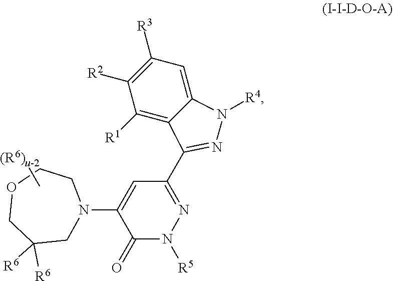

or a pharmaceutically acceptable salt, stereoisomer, solvate, prodrug, isotopic derivative, or tautomer thereof.

In some embodiments, the compound is of Formula (I-I-D-O-A-H):

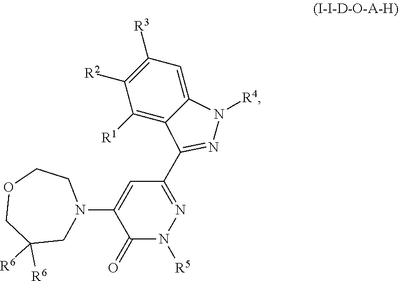

or a pharmaceutically acceptable salt, stereoisomer, solvate, prodrug, isotopic derivative, or tautomer thereof.

In some embodiments, the compound is of Formula (I-I-D-O-A-1):

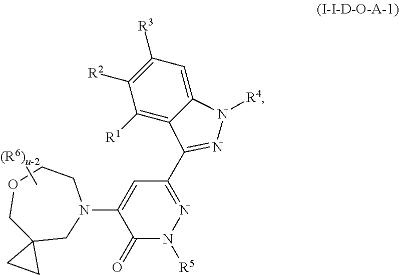

or a pharmaceutically acceptable salt, stereoisomer, solvate, prodrug, isotopic derivative, or tautomer thereof.

In some embodiments, the compound is of Formula (I-I-D-O-A-1-H):

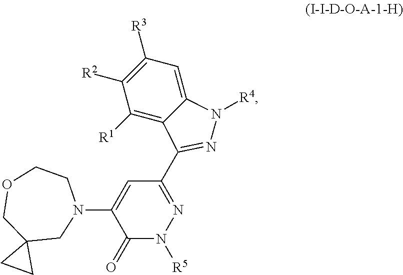

or a pharmaceutically acceptable salt, stereoisomer, solvate, prodrug, isotopic derivative, or tautomer thereof.

In some embodiments, the compound is of Formula (I-I-D-O-A-2):

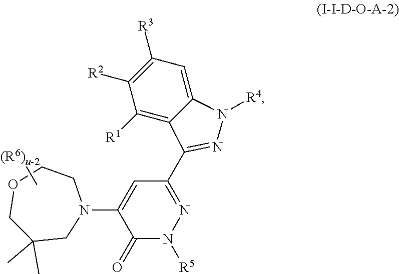

or a pharmaceutically acceptable salt, stereoisomer, solvate, prodrug, isotopic derivative, or tautomer thereof.

In some embodiments, the compound is of Formula (I-I-D-O-A-2-H):

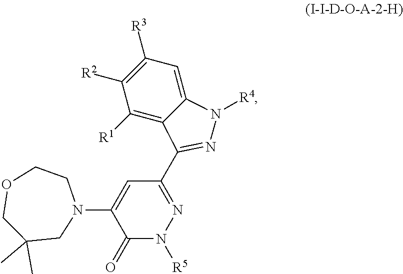

or a pharmaceutically acceptable salt, stereoisomer, solvate, prodrug, isotopic derivative, or tautomer thereof.

In some embodiments, the compound is of Formula (I-I-D-O-A-3):

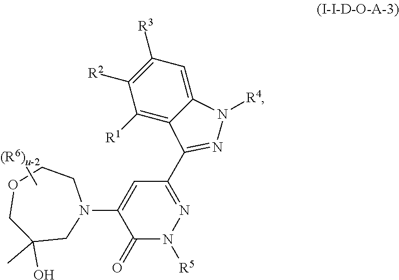

or a pharmaceutically acceptable salt, stereoisomer, solvate, prodrug, isotopic derivative, or tautomer thereof.

In some embodiments, the compound is of Formula (I-I-D-O-3-A-H):

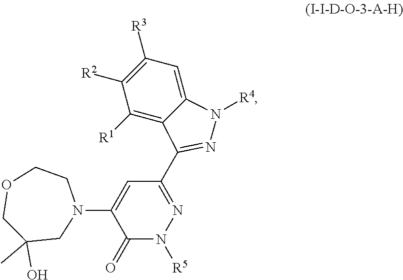

or a pharmaceutically acceptable salt, stereoisomer, solvate, prodrug, isotopic derivative, or tautomer thereof.

In some embodiments, the compound is of Formula (I-I-D-O-B):

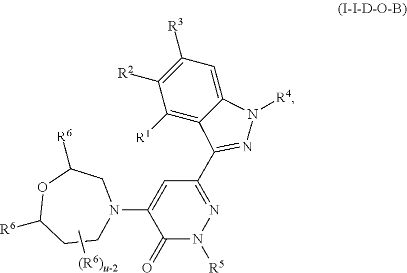

or a pharmaceutically acceptable salt, stereoisomer, solvate, prodrug, isotopic derivative, or tautomer thereof.

In some embodiments, the compound is of Formula (I-I-D-O-B-H):

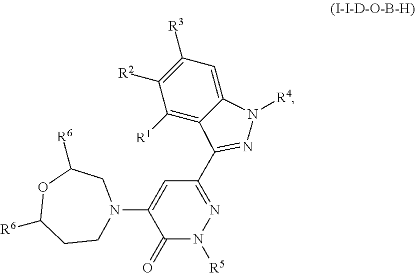

or a pharmaceutically acceptable salt, stereoisomer, solvate, prodrug, isotopic derivative, or tautomer thereof.

In some embodiments, the compound is of Formula (I-I-D-O-B-1):

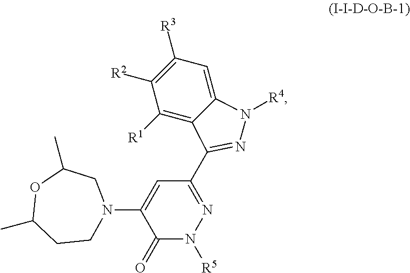

or a pharmaceutically acceptable salt, stereoisomer, solvate, prodrug, isotopic derivative, or tautomer thereof.

In some embodiments, the compound is of Formula (I-I-D-O-B-1*):

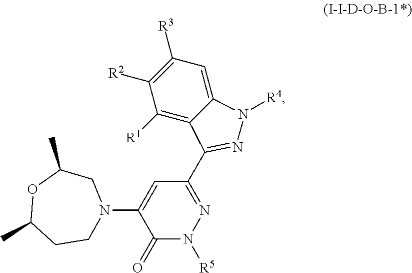

or a pharmaceutically acceptable salt, stereoisomer, solvate, prodrug, isotopic derivative, or tautomer thereof.

In some embodiments, the compound is of Formula (I-I-D-O-B-1**):

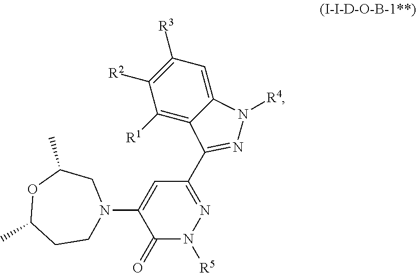

or a pharmaceutically acceptable salt, stereoisomer, solvate, prodrug, isotopic derivative, or tautomer thereof.

In some embodiments, the compound is of Formula (I-I-D-O-B-1***):

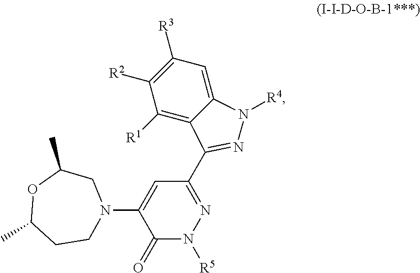

or a pharmaceutically acceptable salt, stereoisomer, solvate, prodrug, isotopic derivative, or tautomer thereof.

In some embodiments, the compound is of Formula (I-I-D-O-B-1****):

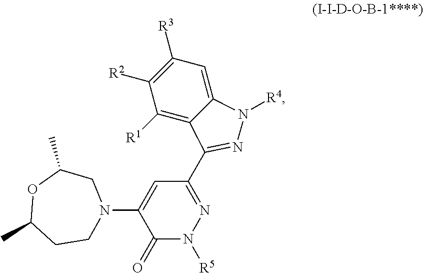

or a pharmaceutically acceptable salt, stereoisomer, solvate, prodrug, isotopic derivative, or tautomer thereof.

In some embodiments, the compound is of Formula (I-I-D-O-C):

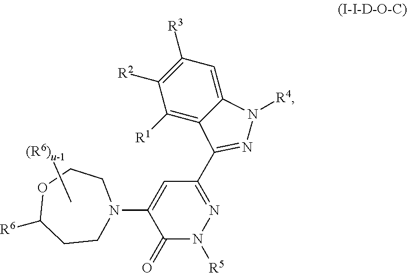

or a pharmaceutically acceptable salt, stereoisomer, solvate, prodrug, isotopic derivative, or tautomer thereof.

In some embodiments, the compound is of Formula (I-I-D-O-C-H):

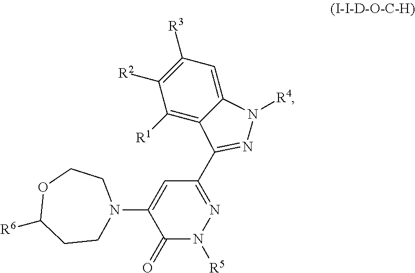

or a pharmaceutically acceptable salt, stereoisomer, solvate, prodrug, isotopic derivative, or tautomer thereof.

In some embodiments, the compound is of Formula (I-I-D-O-C-1):

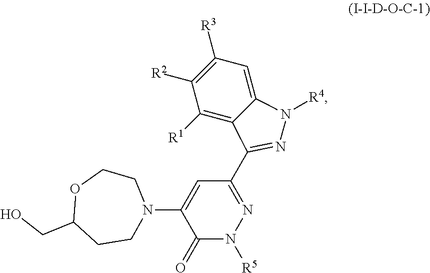

or a pharmaceutically acceptable salt, stereoisomer, solvate, prodrug, isotopic derivative, or tautomer thereof.

In some embodiments, the compound is of Formula (I-I-D-O-C-1*).

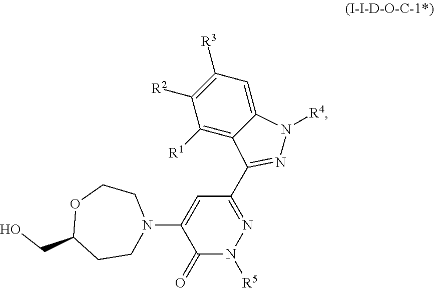

or a pharmaceutically acceptable salt, stereoisomer, solvate, prodrug, isotopic derivative, or tautomer thereof.

In some embodiments, the compound is of Formula (I-I-D-O-C-1**):

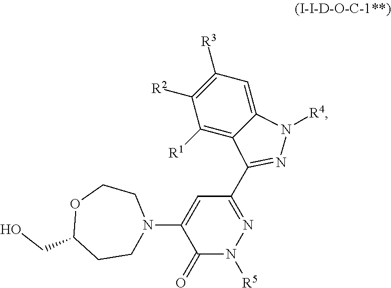

or a pharmaceutically acceptable salt, stereoisomer, solvate, prodrug, isotopic derivative, or tautomer thereof.

In some embodiments, the compound is of Formula (I-I-D-O-D):

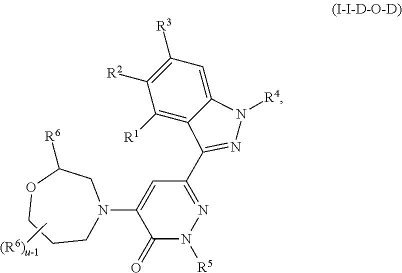

or a pharmaceutically acceptable salt, stereoisomer, solvate, prodrug, isotopic derivative, or tautomer thereof.

In some embodiments, the compound is of Formula (I-I-D-O-D-H):

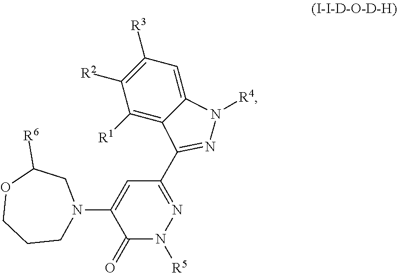

or a pharmaceutically acceptable salt, stereoisomer, solvate, prodrug, isotopic derivative, or tautomer thereof.

In some embodiments, the compound is of Formula (I-I-D-O-D-1):

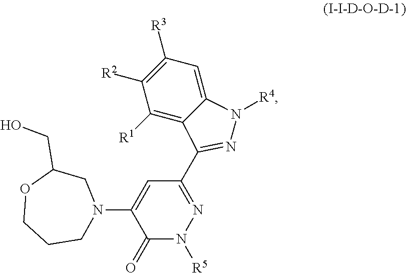

or a pharmaceutically acceptable salt, stereoisomer, solvate, prodrug, isotopic derivative, or tautomer thereof.

In some embodiments, the compound is of Formula (I-I-D-O-D-1*).

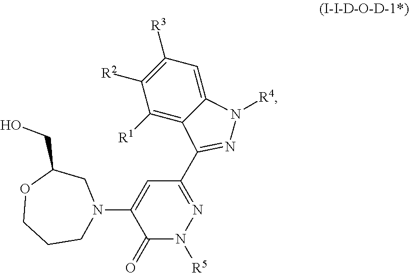

or a pharmaceutically acceptable salt, stereoisomer, solvate, prodrug, isotopic derivative, or tautomer thereof. In some embodiments, the compound is of Formula (I-I-D-O-D-1):

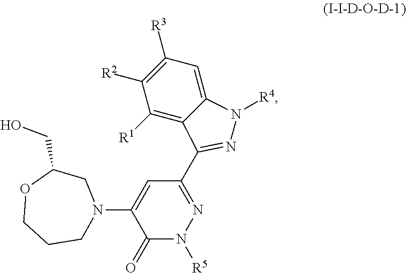

or a pharmaceutically acceptable salt, stereoisomer, solvate, prodrug, isotopic derivative, or tautomer thereof.

In some embodiments, the compound is of Formula (I-I-D-O-E):

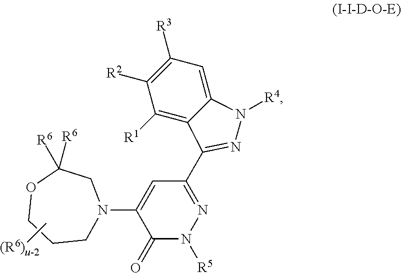

or a pharmaceutically acceptable salt, stereoisomer, solvate, prodrug, isotopic derivative, or tautomer thereof.

In some embodiments, the compound is of Formula (I-I-D-O-E-H):

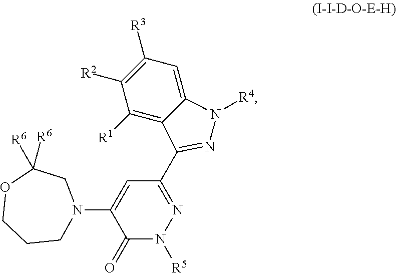

or a pharmaceutically acceptable salt, stereoisomer, solvate, prodrug, isotopic derivative, or tautomer thereof.

In some embodiments, the compound is of Formula (I-I-D-O-E-1):

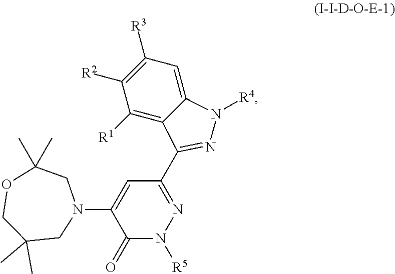

or a pharmaceutically acceptable salt, stereoisomer, solvate, prodrug, isotopic derivative, or tautomer thereof.

In some embodiments, the compound is of Formula (I-I-D-O-F):

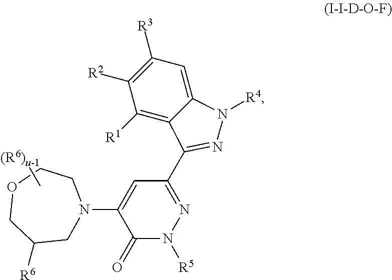

or a pharmaceutically acceptable salt, stereoisomer, solvate, prodrug, isotopic derivative, or tautomer thereof.

In some embodiments, the compound is of Formula (I-I-D-O-F-H):

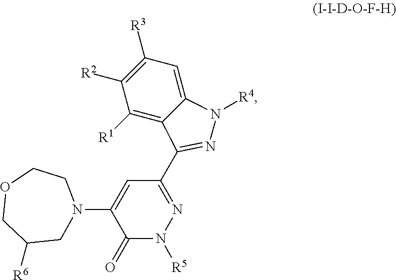

or a pharmaceutically acceptable salt, stereoisomer, solvate, prodrug, isotopic derivative, or tautomer thereof.

In some embodiments, the compound is of Formula (I-I-D-O-F-1):

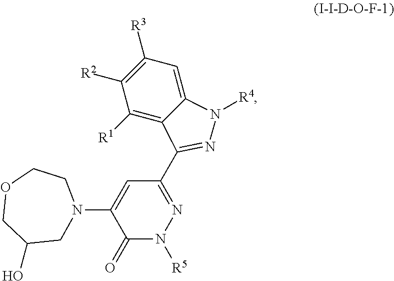

or a pharmaceutically acceptable salt, stereoisomer, solvate, prodrug, isotopic derivative, or tautomer thereof.

In some embodiments, the compound is of Formula (I-I-D-O-F-1*):

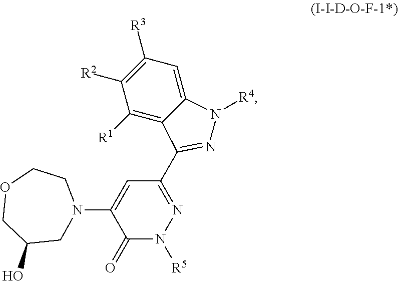

or a pharmaceutically acceptable salt, stereoisomer, solvate, prodrug, isotopic derivative, or tautomer thereof.

In some embodiments, the compound is of Formula (I-I-D-O-F-1**):

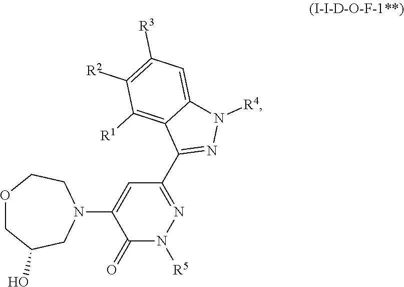

or a pharmaceutically acceptable salt, stereoisomer, solvate, prodrug, isotopic derivative, or tautomer thereof.

In some embodiments, the compound is of Formula (I-I-D-O-F-2):

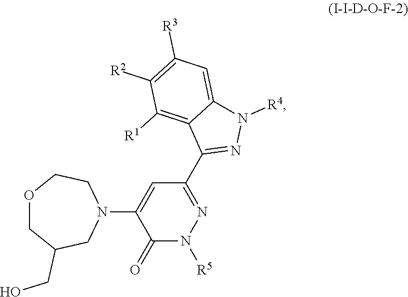

or a pharmaceutically acceptable salt, stereoisomer, solvate, prodrug, isotopic derivative, or tautomer thereof.

In some embodiments, the compound is of Formula (I-I-D-O-F-2*):

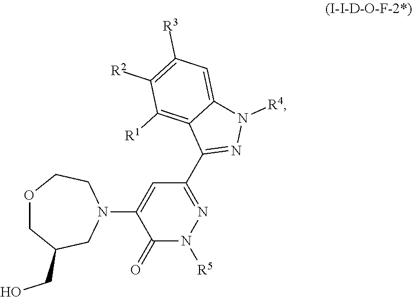

or a pharmaceutically acceptable salt, stereoisomer, solvate, prodrug, isotopic derivative, or tautomer thereof.

In some embodiments, the compound is of Formula (I-I-D-O-F-2):

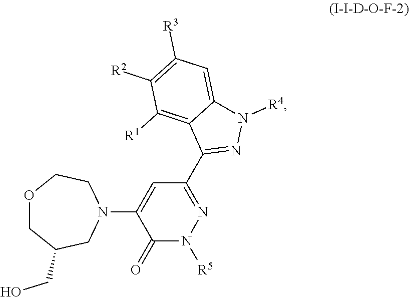

or a pharmaceutically acceptable salt, stereoisomer, solvate, prodrug, isotopic derivative, or tautomer thereof.

In some embodiments, the compound is of Formula (I-I-D-II):

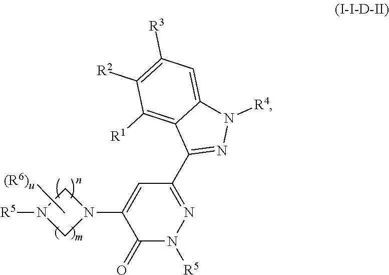

or a pharmaceutically acceptable salt, stereoisomer, solvate, prodrug, isotopic derivative, or tautomer thereof.

In some embodiments, the compound is of Formula (I-I-D-Z):

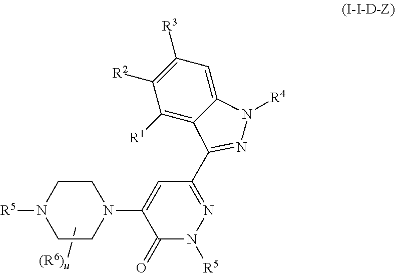

or a pharmaceutically acceptable salt, stereoisomer, solvate, prodrug, isotopic derivative, or tautomer thereof.

In some embodiments, the compound is of Formula (I-I-D-Z-H):

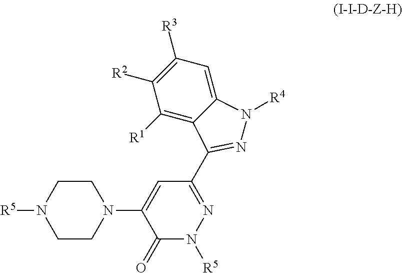

or a pharmaceutically acceptable salt, stereoisomer, solvate, prodrug, isotopic derivative, or tautomer thereof.

In some embodiments, the compound is of Formula (I-I-D-Z-0):

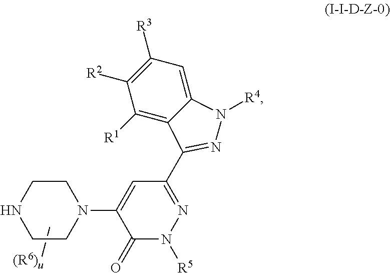

or a pharmaceutically acceptable salt, stereoisomer, solvate, prodrug, isotopic derivative, or tautomer thereof.

In some embodiments, the compound is of Formula (I-I-D-Z-1):

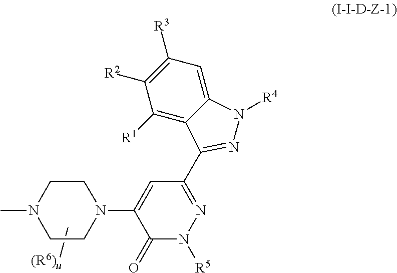

or a pharmaceutically acceptable salt, stereoisomer, solvate, prodrug, isotopic derivative, or tautomer thereof.

In some embodiments, the compound is of Formula (I-I-D-Z-1-H):

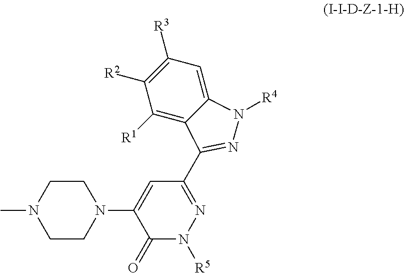

or a pharmaceutically acceptable salt, stereoisomer, solvate, prodrug, isotopic derivative, or tautomer thereof.

In some embodiments, the compound is of Formula (I-I-D-Z-1-A):

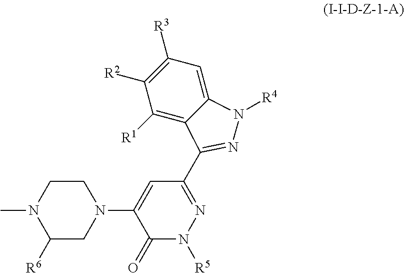

or a pharmaceutically acceptable salt, stereoisomer, solvate, prodrug, isotopic derivative, or tautomer thereof.

In some embodiments, the compound is of Formula (I-I-D-Z-1-a):

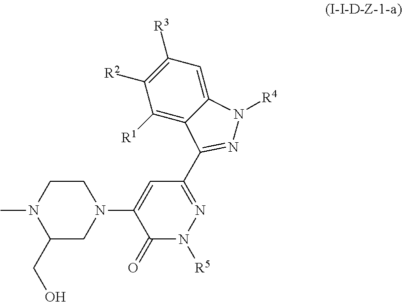

or a pharmaceutically acceptable salt, stereoisomer, solvate, prodrug, isotopic derivative, or tautomer thereof.

In some embodiments, the compound is of Formula (I-I-D-Z-1-a*):

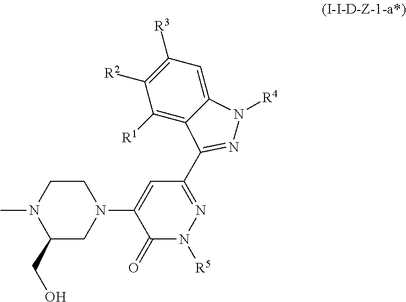

or a pharmaceutically acceptable salt, stereoisomer, solvate, prodrug, isotopic derivative, or tautomer thereof.

In some embodiments, the compound is of Formula (I-I-D-Z-1-a**):

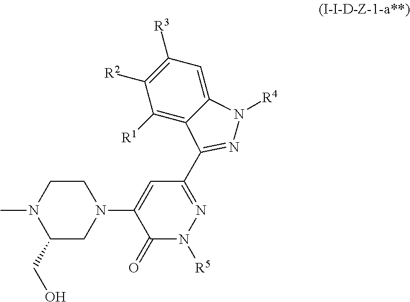

or a pharmaceutically acceptable salt, stereoisomer, solvate, prodrug, isotopic derivative, or tautomer thereof.

In some embodiments, the compound is of Formula (I-I-D-D):

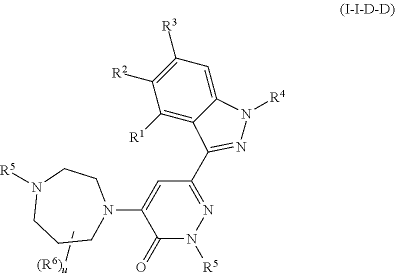

or a pharmaceutically acceptable salt, stereoisomer, solvate, prodrug, isotopic derivative, or tautomer thereof.

In some embodiments, the compound is of Formula (I-I-D-D-H):

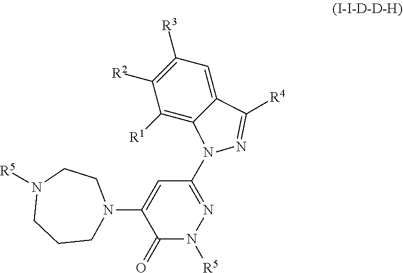

or a pharmaceutically acceptable salt, stereoisomer, solvate, prodrug, isotopic derivative, or tautomer thereof.

In some embodiments, the compound is of Formula (I-I-D-D-0):

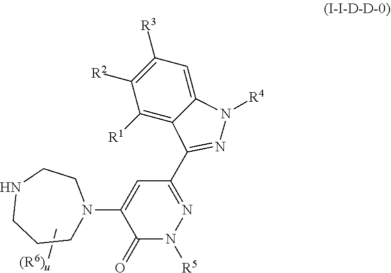

or a pharmaceutically acceptable salt, stereoisomer, solvate, prodrug, isotopic derivative, or tautomer thereof.

In some embodiments, the compound is of Formula (I-I-D-D-0-H):

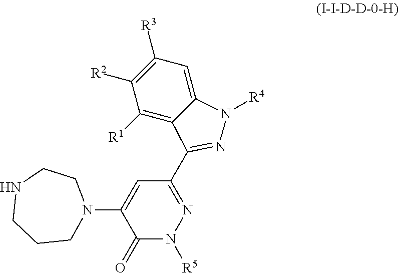

or a pharmaceutically acceptable salt, stereoisomer, solvate, prodrug, isotopic derivative, or tautomer thereof.

In some embodiments, the compound is of Formula (I-I-D-D-0-A):

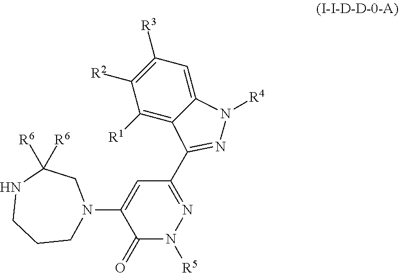

or a pharmaceutically acceptable salt, stereoisomer, solvate, prodrug, isotopic derivative, or tautomer thereof.

In some embodiments, the compound is of Formula (I-I-D-D-0-a):

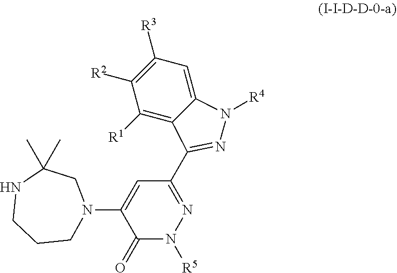

or a pharmaceutically acceptable salt, stereoisomer, solvate, prodrug, isotopic derivative, or tautomer thereof.

In some embodiments, the compound is of Formula (I-I-D-D-0-B):

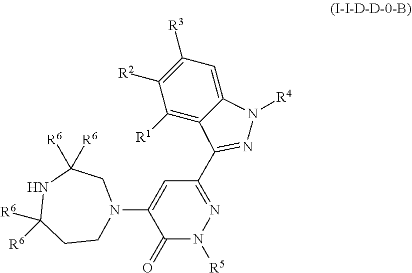

or a pharmaceutically acceptable salt, stereoisomer, solvate, prodrug, isotopic derivative, or tautomer thereof.

In some embodiments, the compound is of Formula (I-I-D-D-0-b):

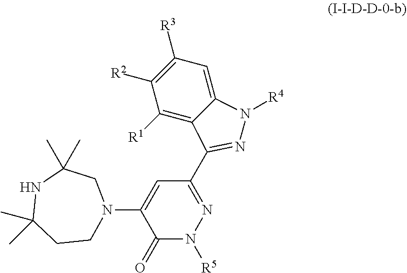

or a pharmaceutically acceptable salt, stereoisomer, solvate, prodrug, isotopic derivative, or tautomer thereof.

In some embodiments, the compound is of Formula (I-I-D-D-0-C):

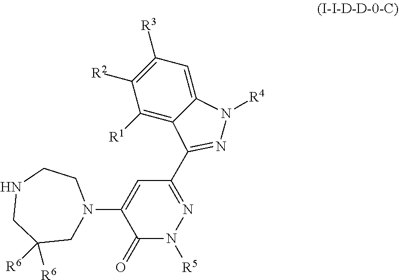

or a pharmaceutically acceptable salt, stereoisomer, solvate, prodrug, isotopic derivative, or tautomer thereof.

In some embodiments, the compound is of Formula (I-I-D-D-0-c):

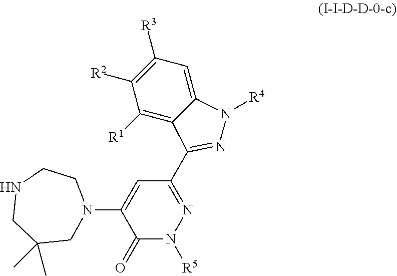

or a pharmaceutically acceptable salt, stereoisomer, solvate, prodrug, isotopic derivative, or tautomer thereof.

In some embodiments, the compound is of Formula (I-I-D-D-0-cc):

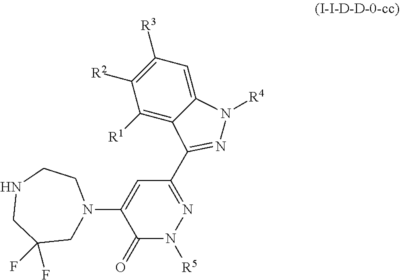

or a pharmaceutically acceptable salt, stereoisomer, solvate, prodrug, isotopic derivative, or tautomer thereof.

In some embodiments, the compound is of Formula (I-I-D-D-0-ccc):

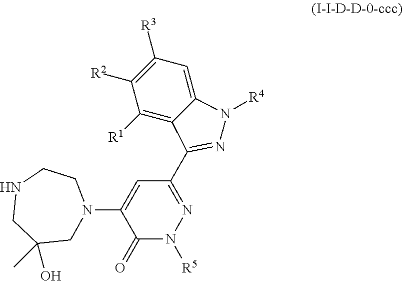

or a pharmaceutically acceptable salt, stereoisomer, solvate, prodrug, isotopic derivative, or tautomer thereof.

In some embodiments, the compound is of Formula (I-I-D-D-0-D):

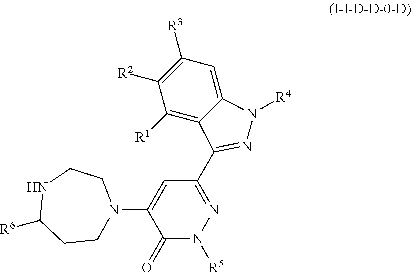

or a pharmaceutically acceptable salt, stereoisomer, solvate, prodrug, isotopic derivative, or tautomer thereof.

In some embodiments, the compound is of Formula (I-I-D-D-0-d):

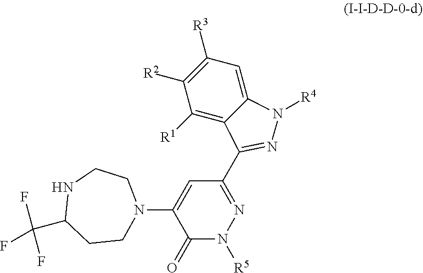

or a pharmaceutically acceptable salt, stereoisomer, solvate, prodrug, isotopic derivative, or tautomer thereof.

In some embodiments, the compound is of Formula (I-I-D-D-1):

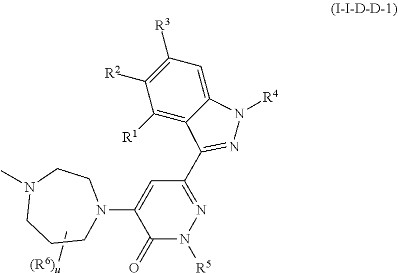

or a pharmaceutically acceptable salt, stereoisomer, solvate, prodrug, isotopic derivative, or tautomer thereof.

In some embodiments, the compound is of Formula (I-I-D-D-1-H):

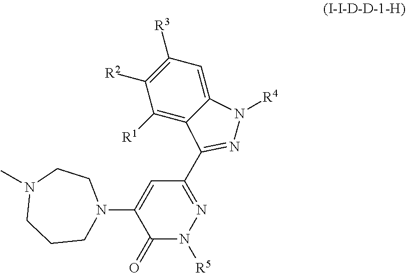

or a pharmaceutically acceptable salt, stereoisomer, solvate, prodrug, isotopic derivative, or tautomer thereof.

In some embodiments, the compound is of Formula (I-I-D-D-1-A):

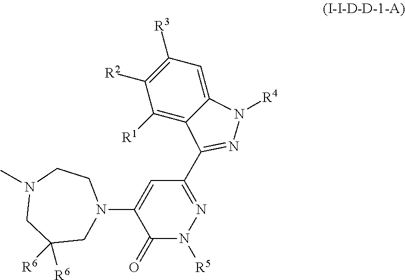

or a pharmaceutically acceptable salt, stereoisomer, solvate, prodrug, isotopic derivative, or tautomer thereof.

In some embodiments, the compound is of Formula (I-I-D-D-1-a):

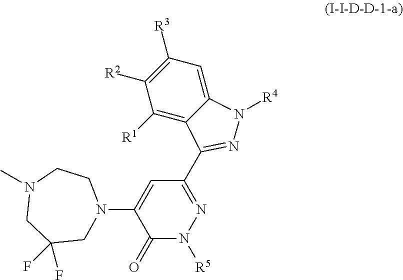

or a pharmaceutically acceptable salt, stereoisomer, solvate, prodrug, isotopic derivative, or tautomer thereof.

In some embodiments, the compound is of Formula (I-I-D-D-1-B):

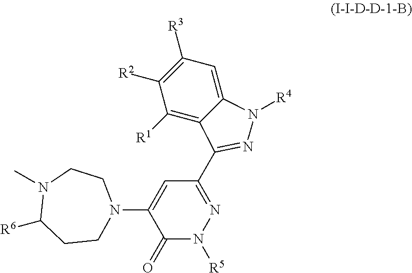

or a pharmaceutically acceptable salt, stereoisomer, solvate, prodrug, isotopic derivative, or tautomer thereof.

In some embodiments, the compound is of Formula (I-I-D-D-1-b):

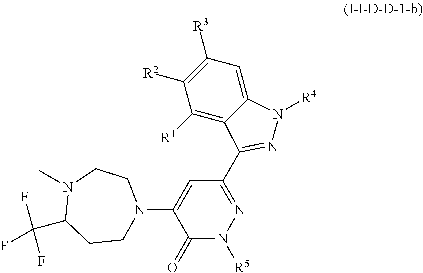

or a pharmaceutically acceptable salt, stereoisomer, solvate, prodrug, isotopic derivative, or tautomer thereof.

In some embodiments, the compound is of Formula (I-I-D-D-1-C):

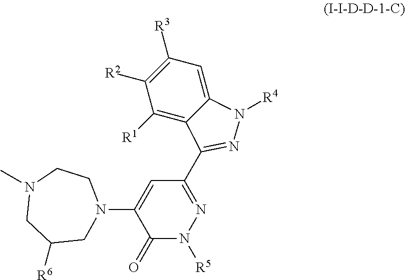

or a pharmaceutically acceptable salt, stereoisomer, solvate, prodrug, isotopic derivative, or tautomer thereof.

In some embodiments, the compound is of Formula (I-I-D-D-1-c):

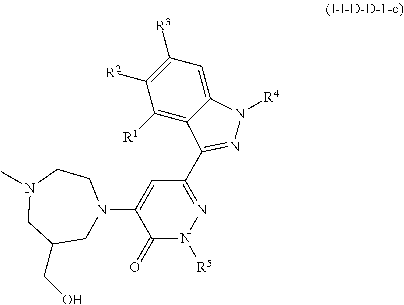

or a pharmaceutically acceptable salt, stereoisomer, solvate, prodrug, isotopic derivative, or tautomer thereof.

In some embodiments, the compound is of Formula (I-I-D-D-1-cc):

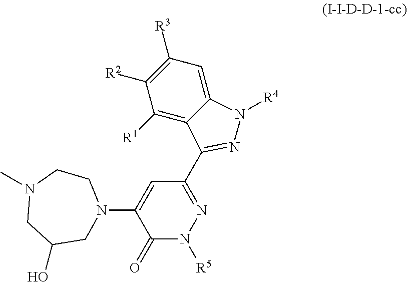

or a pharmaceutically acceptable salt, stereoisomer, solvate, prodrug, isotopic derivative, or tautomer thereof.

In some embodiments, the compound is of Formula (I-I-D-D-2):

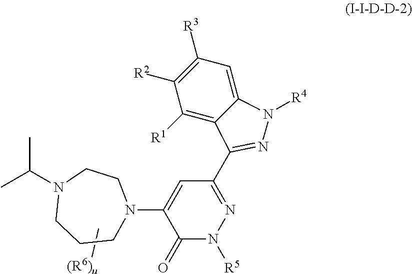

or a pharmaceutically acceptable salt, stereoisomer, solvate, prodrug, isotopic derivative, or tautomer thereof.

In some embodiments, the compound is of Formula (I-I-D-D-2-H):

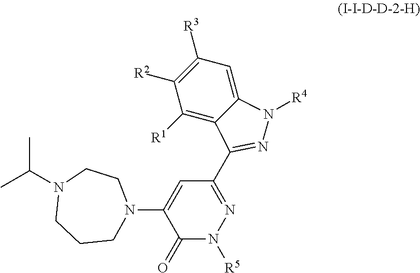

or a pharmaceutically acceptable salt, stereoisomer, solvate, prodrug, isotopic derivative, or tautomer thereof.

In some embodiments, the compound is of Formula (I-I-D-D-3):

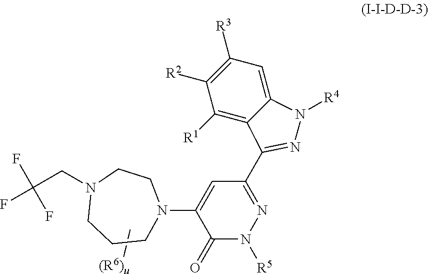

or a pharmaceutically acceptable salt, stereoisomer, solvate, prodrug, isotopic derivative, or tautomer thereof.

In some embodiments, the compound is of Formula (I-I-D-D-3-H):

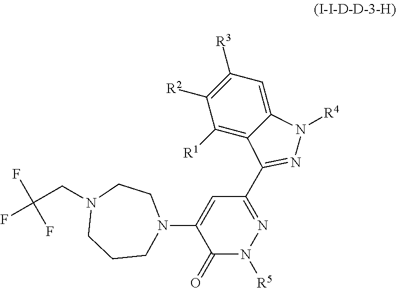

or a pharmaceutically acceptable salt, stereoisomer, solvate, prodrug, isotopic derivative, or tautomer thereof.

In some embodiments, the compound is of Formula (I-I-D-D-4):

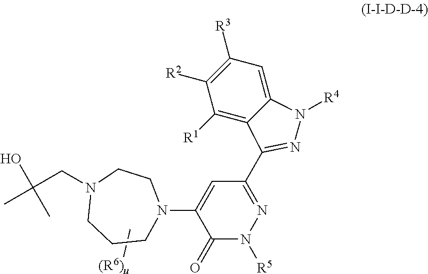

or a pharmaceutically acceptable salt, stereoisomer, solvate, prodrug, isotopic derivative, or tautomer thereof.

In some embodiments, the compound is of Formula (I-I-D-D-4-H):

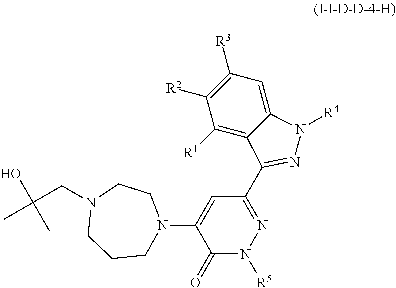

or a pharmaceutically acceptable salt, stereoisomer, solvate, prodrug, isotopic derivative, or tautomer thereof.

In some embodiments, the compound is of Formula (I-I-D-II):

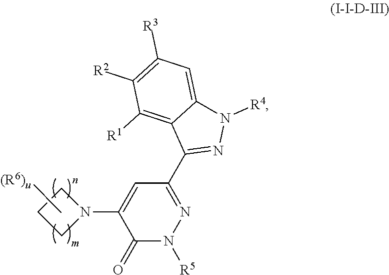

or a pharmaceutically acceptable salt, stereoisomer, solvate, prodrug, isotopic derivative, or tautomer thereof.

In some embodiments, the compound is of Formula (I-I-D-P):

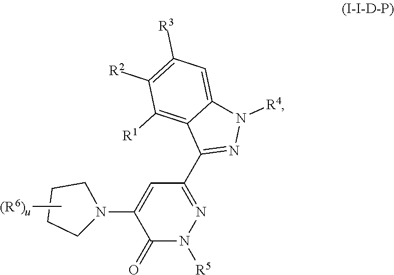

or a pharmaceutically acceptable salt, stereoisomer, solvate, prodrug, isotopic derivative, or tautomer thereof.

In some embodiments, the compound is of Formula (I-I-D-P-H):

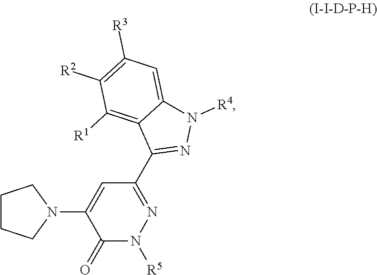

or a pharmaceutically acceptable salt, stereoisomer, solvate, prodrug, isotopic derivative, or tautomer thereof.

In some embodiments, the compound is of Formula (I-I-D-P-0):

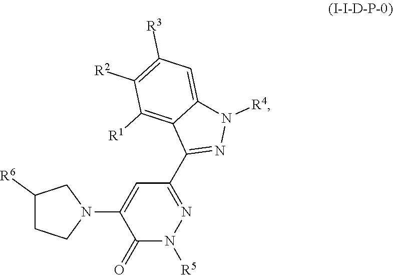

or a pharmaceutically acceptable salt, stereoisomer, solvate, prodrug, isotopic derivative, or tautomer thereof.

In some embodiments, the compound is of Formula (I-I-D-P-0-1):

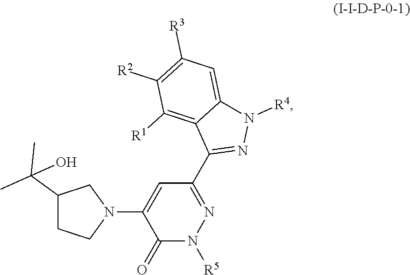

or a pharmaceutically acceptable salt, stereoisomer, solvate, prodrug, isotopic derivative, or tautomer thereof.

In some embodiments, the compound is of Formula (I-I-D-P-1):

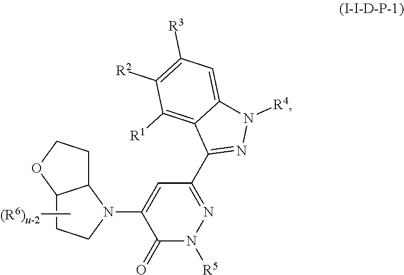

or a pharmaceutically acceptable salt, stereoisomer, solvate, prodrug, isotopic derivative, or tautomer thereof.

In some embodiments, the compound is of Formula (I-I-D-P-1-H):

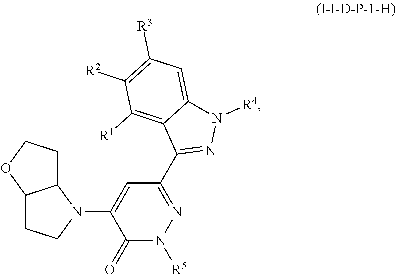

or a pharmaceutically acceptable salt, stereoisomer, solvate, prodrug, isotopic derivative, or tautomer thereof.

In some embodiments, the compound is of Formula (I-I-D-P-2):

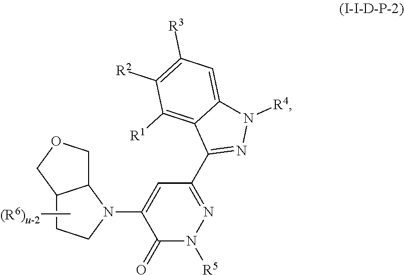

or a pharmaceutically acceptable salt, stereoisomer, solvate, prodrug, isotopic derivative, or tautomer thereof.

In some embodiments, the compound is of Formula (I-I-D-P-2-H):

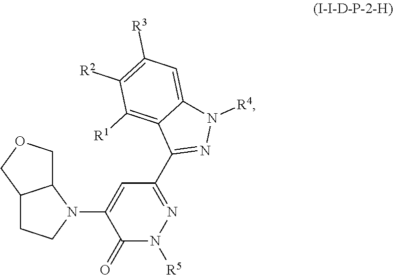

or a pharmaceutically acceptable salt, stereoisomer, solvate, prodrug, isotopic derivative, or tautomer thereof.

In some embodiments, the compound is of Formula (I-I-D-P-2-1):

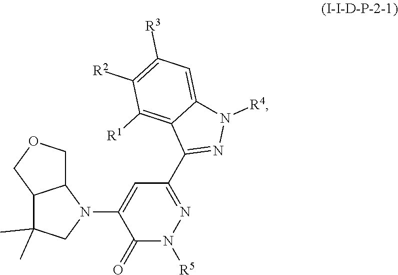

or a pharmaceutically acceptable salt, stereoisomer, solvate, prodrug, isotopic derivative, or tautomer thereof.

In some embodiments, the compound is of Formula (I-I-D-P-3):

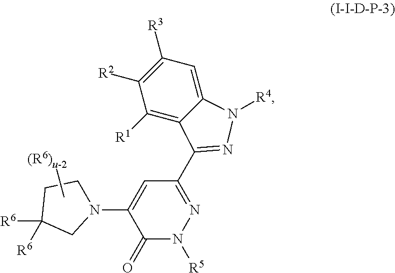

or a pharmaceutically acceptable salt, stereoisomer, solvate, prodrug, isotopic derivative, or tautomer thereof.

In some embodiments, the compound is of Formula (I-I-D-P-3-H):

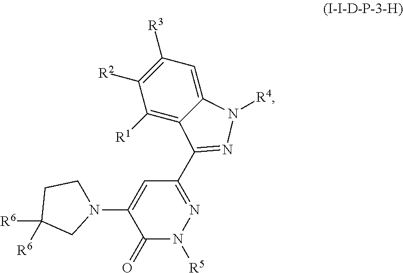

or a pharmaceutically acceptable salt, stereoisomer, solvate, prodrug, isotopic derivative, or tautomer thereof.

In some embodiments, the compound is of Formula (I-I-D-P-3-1):

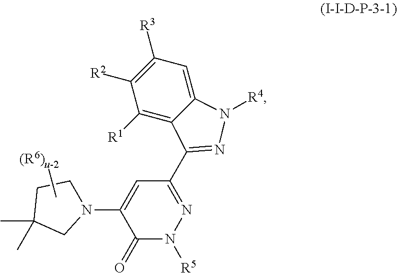

or a pharmaceutically acceptable salt, stereoisomer, solvate, prodrug, isotopic derivative, or tautomer thereof.

In some embodiments, the compound is of Formula (I-I-D-P-3-1-a):

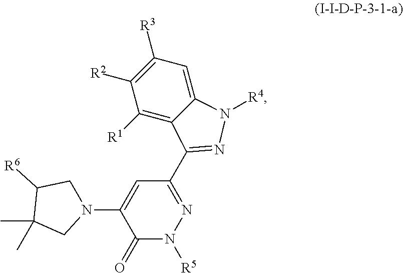

or a pharmaceutically acceptable salt, stereoisomer, solvate, prodrug, isotopic derivative, or tautomer thereof.

In some embodiments, the compound is of Formula (I-I-D-P-3-1-a-1):

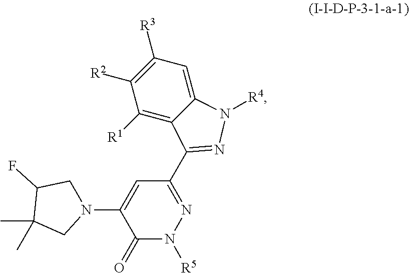

or a pharmaceutically acceptable salt, stereoisomer, solvate, prodrug, isotopic derivative, or tautomer thereof.

In some embodiments, the compound is of Formula (I-I-D-P-3-1-a-2):

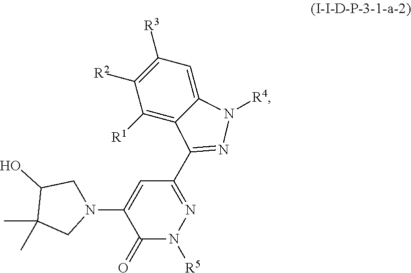

or a pharmaceutically acceptable salt, stereoisomer, solvate, prodrug, isotopic derivative, or tautomer thereof.

In some embodiments, the compound is of Formula (I-I-D-P-3-2):

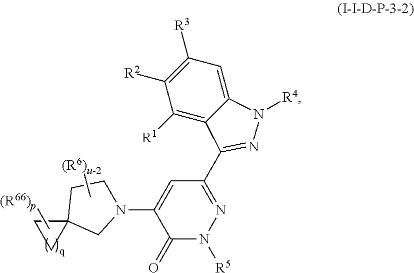

or a pharmaceutically acceptable salt, stereoisomer, solvate, prodrug, isotopic derivative, or tautomer thereof, wherein q is an integer selected from 1, 2, 3, 4 and 5; p is an integer selected from 0, 1, and 2; R⁶⁶ is selected from halogen, OH, CN, C₁-C₆ alkyl, C₁-C₆ alkoxy, C₁-C₆ hydroxyalkyl, C₁-C₆ alkyl-C₁-C₆ alkoxy, S(O)₂R¹²; and all other variables are as described herein.

In some embodiments, the compound is of Formula (I-I-D-P-3-2-a):

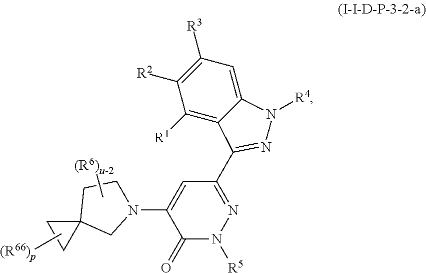

or a pharmaceutically acceptable salt, stereoisomer, solvate, prodrug, isotopic derivative, or tautomer thereof, wherein p is an integer selected from 0, 1, and 2; R⁶⁶ is selected from halogen, OH, CN, C₁-C₆ alkyl, C₁-C₆ alkoxy, C₁-C₆ hydroxyalkyl, C₁-C₆ alkyl-C₁-C₆ alkoxy, S(O)₂R¹²; and all other variables are as described herein.

In some embodiments, the compound is of Formula (I-I-D-P-3-2-a-H):

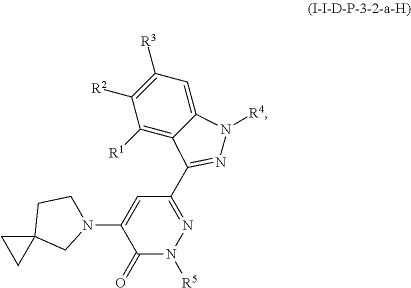

or a pharmaceutically acceptable salt, stereoisomer, solvate, prodrug, isotopic derivative, or tautomer thereof.

In some embodiments, the compound is of Formula (I-I-D-P-3-2-a-1):

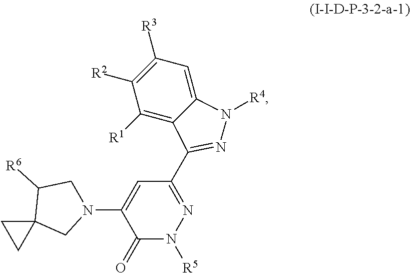

or a pharmaceutically acceptable salt, stereoisomer, solvate, prodrug, isotopic derivative, or tautomer thereof.

In some embodiments, the compound is of Formula (I-I-D-P-3-2-a-1-a):

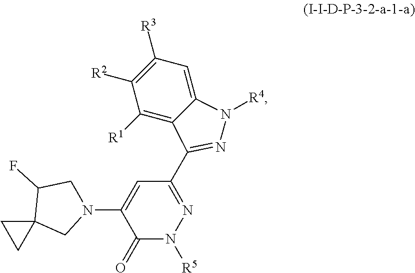

or a pharmaceutically acceptable salt, stereoisomer, solvate, prodrug, isotopic derivative, or tautomer thereof.

In some embodiments, the compound is of Formula (I-I-D-P-3-2-b):

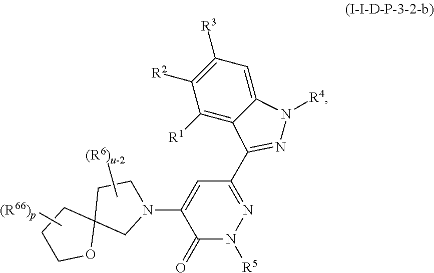

or a pharmaceutically acceptable salt, stereoisomer, solvate, prodrug, isotopic derivative, or tautomer thereof, wherein p is an integer selected from 0, 1, and 2; R⁶⁶ is selected from halogen, OH, CN, C₁-C₆ alkyl, C₁-C₆ alkoxy, C₁-C₆ hydroxyalkyl, C₁-C₆ alkyl-C₁-C₆ alkoxy, S(O)₂R¹²; and all other variables are as described herein.

In some embodiments, the compound is of Formula (I-I-D-P-3-2-b-H):

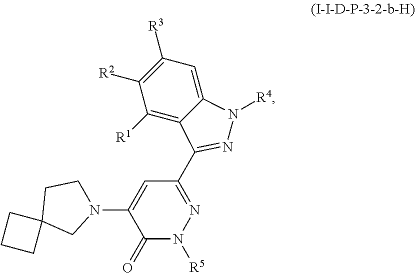

or a pharmaceutically acceptable salt, stereoisomer, solvate, prodrug, isotopic derivative, or tautomer thereof.

In some embodiments, the compound is of Formula (I-I-D-P-3-2-b-1):

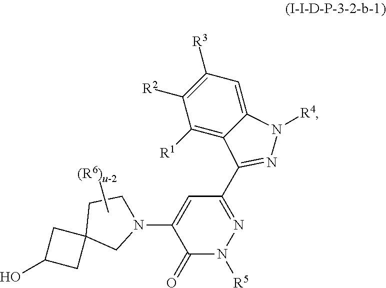

or a pharmaceutically acceptable salt, stereoisomer, solvate, prodrug, isotopic derivative, or tautomer thereof.

In some embodiments, the compound is of Formula (I-I-D-P-3-2-b-1-H):

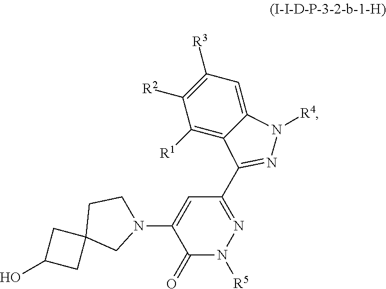

or a pharmaceutically acceptable salt, stereoisomer, solvate, prodrug, isotopic derivative, or tautomer thereof.

In some embodiments, the compound is of Formula (I-I-D-P-3-2-b-2):

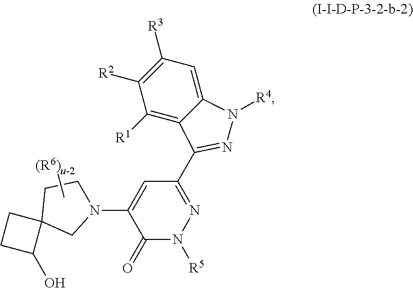

or a pharmaceutically acceptable salt, stereoisomer, solvate, prodrug, isotopic derivative, or tautomer thereof.

In some embodiments, the compound is of Formula (I-I-D-P-3-2-b-2-H):

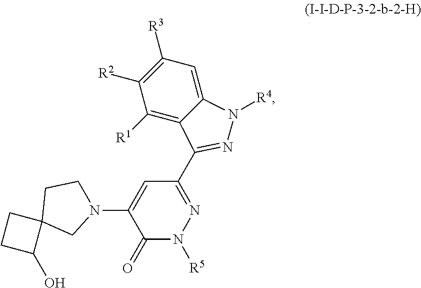

or a pharmaceutically acceptable salt, stereoisomer, solvate, prodrug, isotopic derivative, or tautomer thereof.

In some embodiments, the compound is of Formula (I-I-D-P-3-2-b-3):

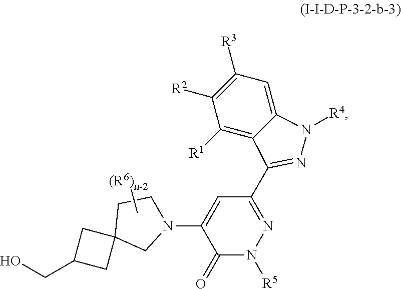

or a pharmaceutically acceptable salt, stereoisomer, solvate, prodrug, isotopic derivative, or tautomer thereof.

In some embodiments, the compound is of Formula (I-I-D-P-3-2-b-3-H):

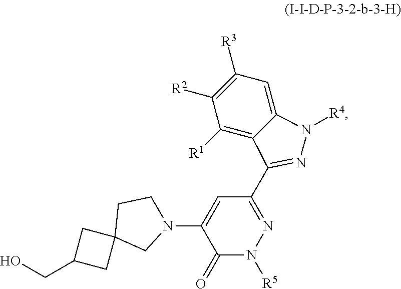

or a pharmaceutically acceptable salt, stereoisomer, solvate, prodrug, isotopic derivative, or tautomer thereof.

In some embodiments, the compound is of Formula (I-I-D-P-3-2-b-4):

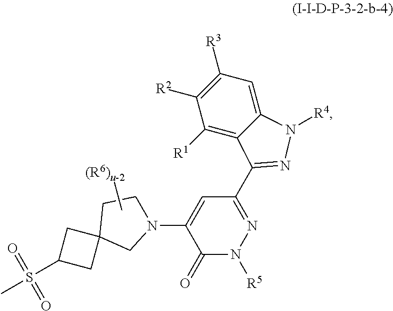

or a pharmaceutically acceptable salt, stereoisomer, solvate, prodrug, isotopic derivative, or tautomer thereof.

In some embodiments, the compound is of Formula (I-I-D-P-3-2-b-4-H):

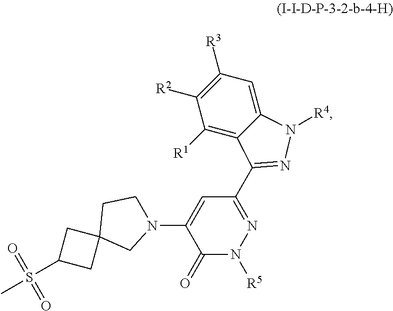

or a pharmaceutically acceptable salt, stereoisomer, solvate, prodrug, isotopic derivative, or tautomer thereof.

In some embodiments, the compound is of Formula (I-I-D-P-3-2-b-5):

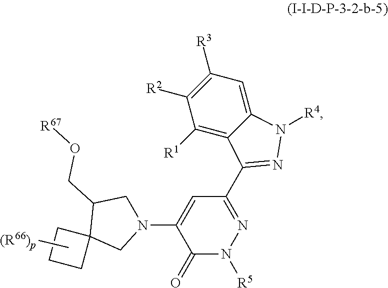

or a pharmaceutically acceptable salt, stereoisomer, solvate, prodrug, isotopic derivative, or tautomer thereof, wherein p is an integer selected from 0, 1, and 2; R⁶⁶ is selected from halogen, OH, CN, C₁-C₆ alkyl, C₁-C₆ alkoxy, C₁-C₆ hydroxyalkyl, C₁-C₆ alkyl-C₁-C₆ alkoxy, S(O)₂R¹²; R⁶⁷ is selected from H, C₁-C₆ alkyl, C₃-C₈ cycloalkyl; and all other variables are as described herein.

In some embodiments, the compound is of Formula (I-I-D-P-3-2-b-5-H):

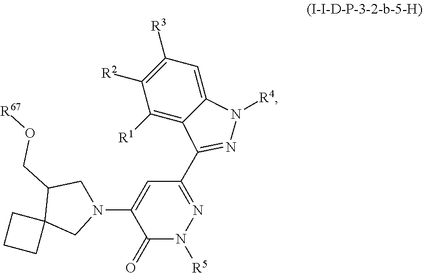

or a pharmaceutically acceptable salt, stereoisomer, solvate, prodrug, isotopic derivative, or tautomer thereof, wherein R⁶⁷ is selected from H, C₁-C₆ alkyl, C₃-C₈ cycloalkyl; and all other variables are as described herein.

In some embodiments, the compound is of Formula (I-I-D-P-3-2-b-5-a):

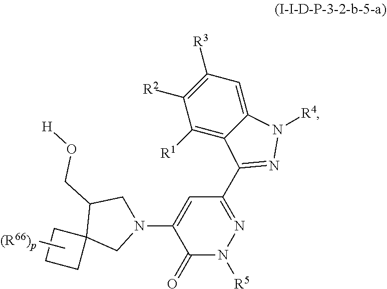

or a pharmaceutically acceptable salt, stereoisomer, solvate, prodrug, isotopic derivative, or tautomer thereof, wherein p is an integer selected from 0, 1, and 2; R⁶⁶ is selected from halogen, OH, CN, C₁-C₆ alkyl, C₁-C₆ alkoxy, C₁-C₆ hydroxyalkyl, C₁-C₆ alkyl-C₁-C₆ alkoxy, S(O)₂R¹²; and all other variables are as described herein.

In some embodiments, the compound is of Formula (I-I-D-P-3-2-b-5-a-H):

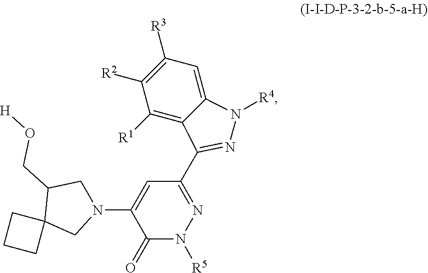

or a pharmaceutically acceptable salt, stereoisomer, solvate, prodrug, isotopic derivative, or tautomer thereof.

In some embodiments, the compound is of Formula (I-I-D-P-3-2-b-5-b):

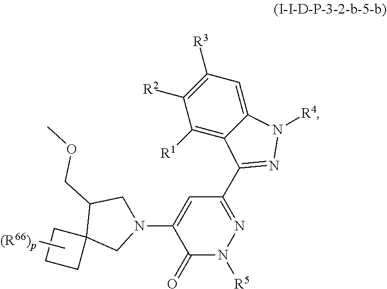

or a pharmaceutically acceptable salt, stereoisomer, solvate, prodrug, isotopic derivative, or tautomer thereof, wherein p is an integer selected from 0, 1, and 2; R⁶⁶ is selected from halogen, OH, CN, C₁-C₆ alkyl, C₁-C₆ alkoxy, C₁-C₆ hydroxyalkyl, C₁-C₆ alkyl-C₁-C₆ alkoxy, S(O)₂R¹²; and all other variables are as described herein.

In some embodiments, the compound is of Formula (I-I-D-P-3-2-b-5-b-H):

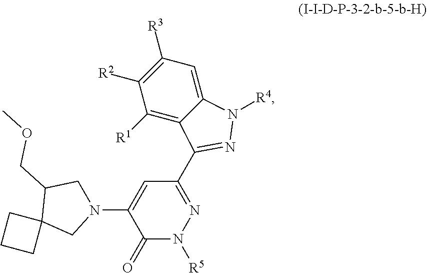

or a pharmaceutically acceptable salt, stereoisomer, solvate, prodrug, isotopic derivative, or tautomer thereof.

In some embodiments, the compound is of Formula (I-I-D-P-3-2-b-5-c):

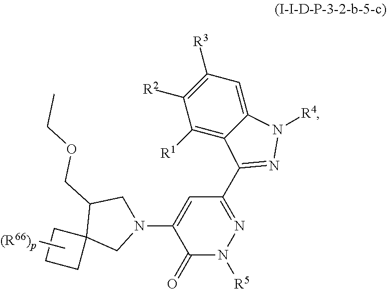

or a pharmaceutically acceptable salt, stereoisomer, solvate, prodrug, isotopic derivative, or tautomer thereof, wherein p is an integer selected from 0, 1, and 2; R⁶⁶ is selected from halogen, OH, CN, C₁-C₆ alkyl, C₁-C₆ alkoxy, C₁-C₆ hydroxyalkyl, C₁-C₆ alkyl-C₁-C₆ alkoxy, S(O)₂R¹²; and all other variables are as described herein.

In some embodiments, the compound is of Formula (I-I-D-P-3-2-b-5-c-H):

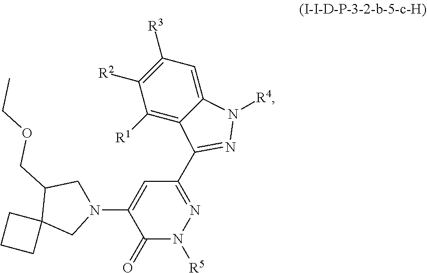

or a pharmaceutically acceptable salt, stereoisomer, solvate, prodrug, isotopic derivative, or tautomer thereof.

In some embodiments, the compound is of Formula (I-I-D-P-3-2-c):

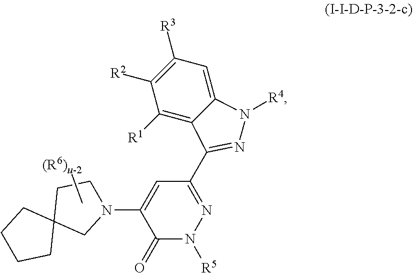

or a pharmaceutically acceptable salt, stereoisomer, solvate, prodrug, isotopic derivative, or tautomer thereof.

In some embodiments, the compound is of Formula (I-I-D-P-3-3):

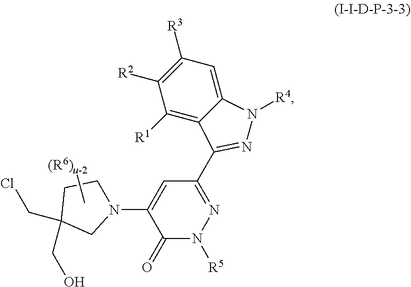

or a pharmaceutically acceptable salt, stereoisomer, solvate, prodrug, isotopic derivative, or tautomer thereof.

In some embodiments, the compound is of Formula (I-I-D-P-3-3-H):

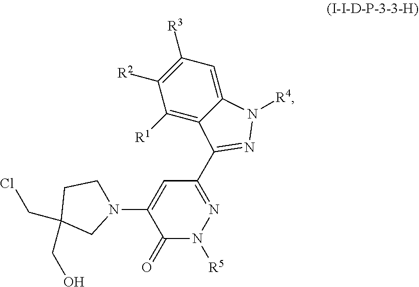

or a pharmaceutically acceptable salt, stereoisomer, solvate, prodrug, isotopic derivative, or tautomer thereof.

In some embodiments, the compound is of Formula (I-I-D-P-3-4):

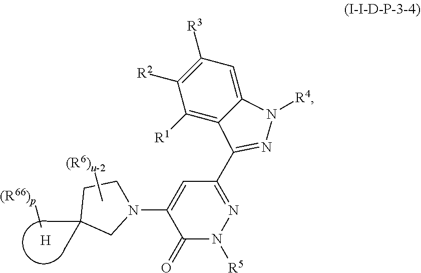

or a pharmaceutically acceptable salt, stereoisomer, solvate, prodrug, isotopic derivative, or tautomer thereof, wherein p is an integer selected from 0, 1, and 2; R⁶⁶ is selected from halogen, OH, CN, C₁-C₆ alkyl, C₁-C₆ alkoxy, C₁-C₆ hydroxyalkyl, C₁-C₆ alkyl-C₁-C₆ alkoxy, S(O)₂R¹²; Ring H is 3-8 membered saturated or partially unsaturated heterocyclyl, comprising 1-3 heteroatoms, selected from N, O, S; and all other variables are as described herein.

In some embodiments, the compound is of Formula (I-I-D-P-3-4-H):

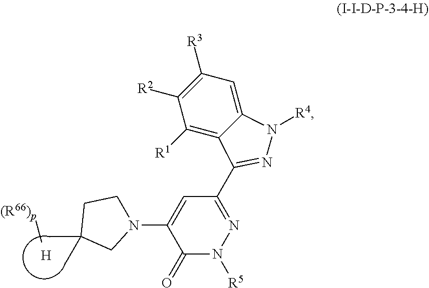

or a pharmaceutically acceptable salt, stereoisomer, solvate, prodrug, isotopic derivative, or tautomer thereof, wherein p is an integer selected from 0, 1, and 2; R⁶⁶ is selected from halogen, OH, CN, C₁-C₆ alkyl, C₁-C₆ alkoxy, C₁-C₆ hydroxyalkyl, C₁-C₆ alkyl-C₁-C₆ alkoxy, S(O)₂R¹²; Ring H is 3-8 membered saturated or partially unsaturated heterocyclyl, comprising 1-3 heteroatoms, selected from N, O, S; and all other variables are as described herein.

In some embodiments, the compound is of Formula (I-I-D-P-3-4-a):

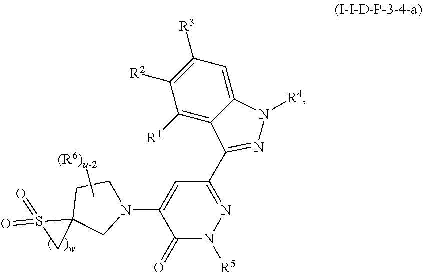

or a pharmaceutically acceptable salt, stereoisomer, solvate, prodrug, isotopic derivative, or tautomer thereof, wherein w is an integer selected from 1, 2, 3, 4 and 5; and all other variables are as described herein.

In some embodiments, the compound is of Formula (I-I-D-P-3-4-a-H):

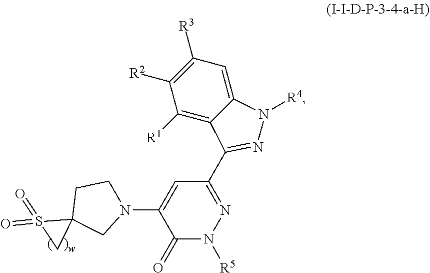

or a pharmaceutically acceptable salt, stereoisomer, solvate, prodrug, isotopic derivative, or tautomer thereof, wherein w is an integer selected from 1, 2, 3, 4 and 5; and all other variables are as described herein.

In some embodiments, the compound is of Formula (I-I-D-P-3-4-a-1):

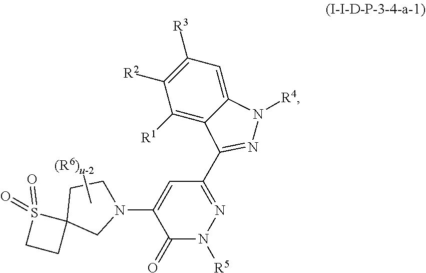

or a pharmaceutically acceptable salt, stereoisomer, solvate, prodrug, isotopic derivative, or tautomer thereof.

In some embodiments, the compound is of Formula (I-I-D-P-3-4-a-H):

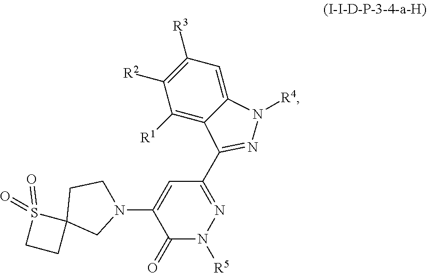

or a pharmaceutically acceptable salt, stereoisomer, solvate, prodrug, isotopic derivative, or tautomer thereof.

In some embodiments, the compound is of Formula (I-I-D-P-3-4-b):

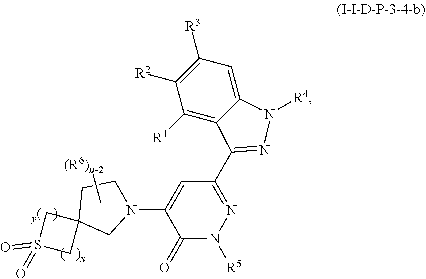

or a pharmaceutically acceptable salt, stereoisomer, solvate, prodrug, isotopic derivative, or tautomer thereof, wherein x and y are integers independently selected from 1, 2, and 3; and all other variables are as described herein.

In some embodiments, the compound is of Formula (I-I-D-P-3-4-b-H):

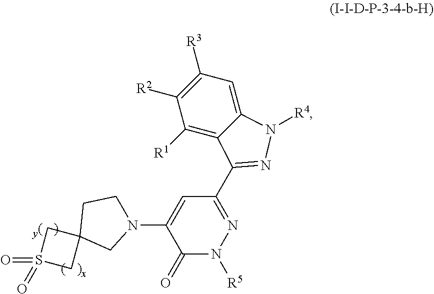

or a pharmaceutically acceptable salt, stereoisomer, solvate, prodrug, isotopic derivative, or tautomer thereof, wherein x and y are integers independently selected from 1, 2, and 3; and all other variables are as described herein.

In some embodiments, the compound is of Formula (I-I-D-P-3-4-b-1):

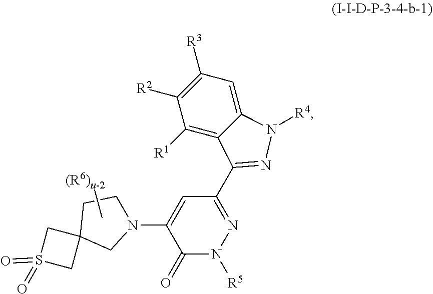

or a pharmaceutically acceptable salt, stereoisomer, solvate, prodrug, isotopic derivative, or tautomer thereof.

In some embodiments, the compound is of Formula (I-I-D-P-3-4-b-1-H):

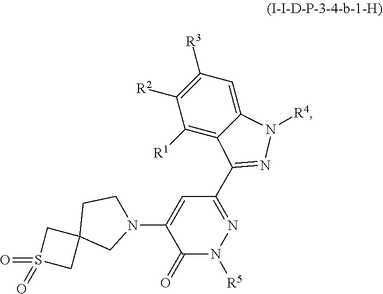

or a pharmaceutically acceptable salt, stereoisomer, solvate, prodrug, isotopic derivative, or tautomer thereof.

In some embodiments, the compound is of Formula (I-I-D-P-3-4-b-2):

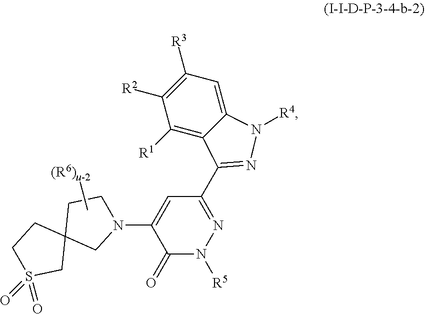

or a pharmaceutically acceptable salt, stereoisomer, solvate, prodrug, isotopic derivative, or tautomer thereof.

In some embodiments, the compound is of Formula (I-I-D-P-3-4-b-2-H):

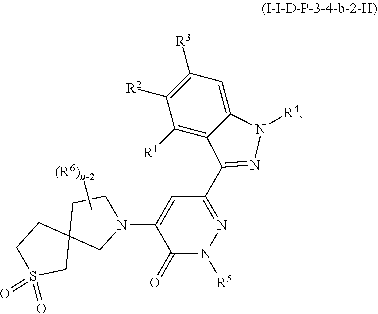

or a pharmaceutically acceptable salt, stereoisomer, solvate, prodrug, isotopic derivative, or tautomer thereof.

In some embodiments, the compound is of Formula (I-I-D-P-3-4-b-3):

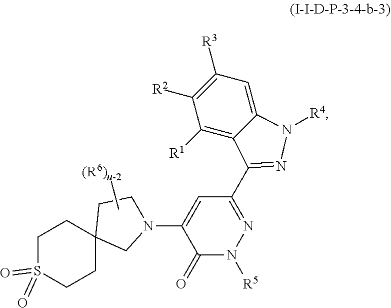

or a pharmaceutically acceptable salt, stereoisomer, solvate, prodrug, isotopic derivative, or tautomer thereof.

In some embodiments, the compound is of Formula (I-I-D-P-3-4-b-3-H):

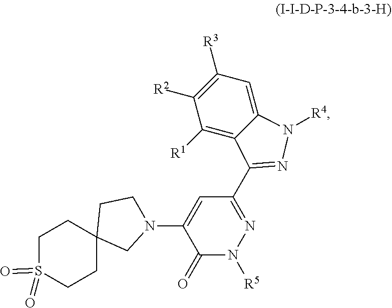

or a pharmaceutically acceptable salt, stereoisomer, solvate, prodrug, isotopic derivative, or tautomer thereof.

In some embodiments, the compound is of Formula (I-I-D-P-3-4-c):

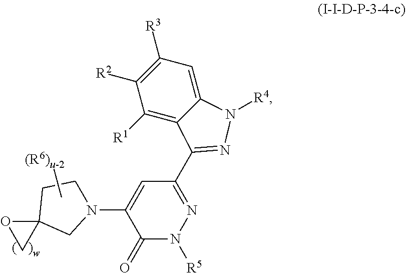

or a pharmaceutically acceptable salt, stereoisomer, solvate, prodrug, isotopic derivative, or tautomer thereof, wherein w is an integer selected from 1, 2, 3, 4 and 5; and all other variables are as described herein.

In some embodiments, the compound is of Formula (I-I-D-P-3-4-c-H):

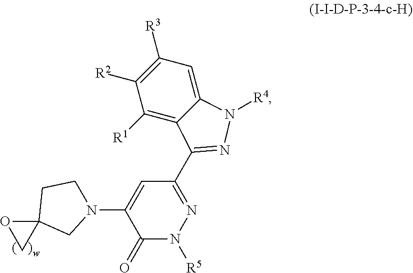

or a pharmaceutically acceptable salt, stereoisomer, solvate, prodrug, isotopic derivative, or tautomer thereof, wherein w is an integer selected from 1, 2, 3, 4 and 5; and all other variables are as described herein.

In some embodiments, the compound is of Formula (I-I-D-P-3-4-c-1):

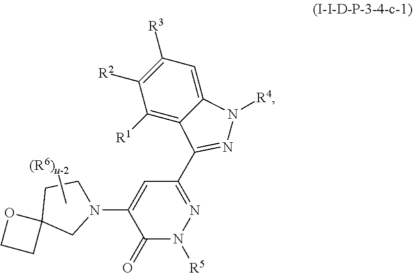

or a pharmaceutically acceptable salt, stereoisomer, solvate, prodrug, isotopic derivative, or tautomer thereof.

In some embodiments, the compound is of Formula (I-I-D-P-3-4-c-1-H):

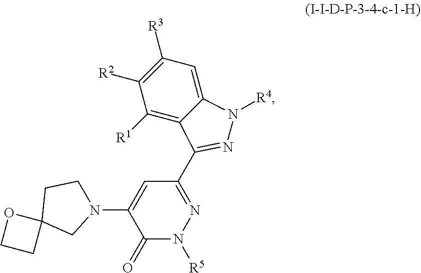

or a pharmaceutically acceptable salt, stereoisomer, solvate, prodrug, isotopic derivative, or tautomer thereof.

In some embodiments, the compound is of Formula (I-I-D-P-3-4-c-2):

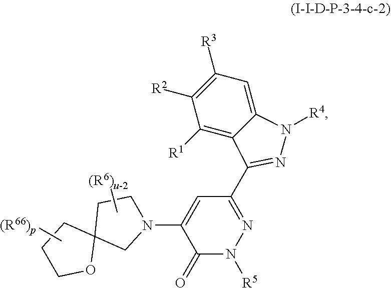

or a pharmaceutically acceptable salt, stereoisomer, solvate, prodrug, isotopic derivative, or tautomer thereof, wherein p is an integer selected from 0, 1, and 2; R⁶⁶ is selected from halogen, OH, CN, C₁-C₆ alkyl, C₁-C₆ alkoxy, C₁-C₆ hydroxyalkyl, C₁-C₆ alkyl-C₁-C₆ alkoxy, S(O)₂R¹²; and all other variables are as described herein.

In some embodiments, the compound is of Formula (I-I-D-P-3-4-c-2-H):

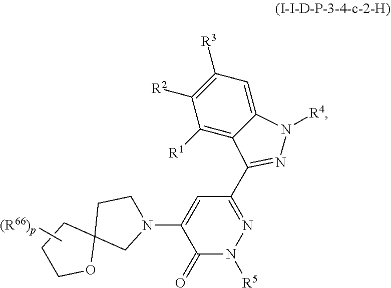

or a pharmaceutically acceptable salt, stereoisomer, solvate, prodrug, isotopic derivative, or tautomer thereof, wherein p is an integer selected from 0, 1, and 2; R⁶⁶ is selected from halogen, OH, CN, C₁-C₆ alkyl, C₁-C₆ alkoxy, C₁-C₆ hydroxyalkyl, C₁-C₆ alkyl-C₁-C₆ alkoxy, S(O)₂R¹²; and all other variables are as described herein.

In some embodiments, the compound is of Formula (I-I-D-P-3-4-c-2-a):

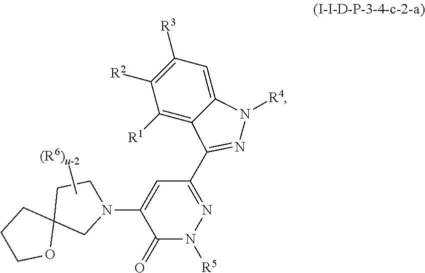

or a pharmaceutically acceptable salt, stereoisomer, solvate, prodrug, isotopic derivative, or tautomer thereof.

In some embodiments, the compound is of Formula (I-I-D-P-3-4-c-2-a-H):

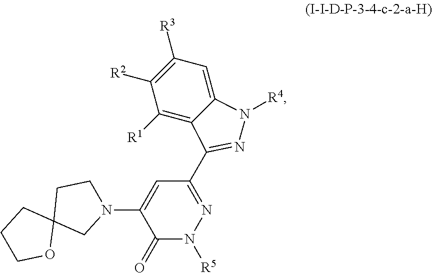

or a pharmaceutically acceptable salt, stereoisomer, solvate, prodrug, isotopic derivative, or tautomer thereof.

In some embodiments, the compound is of Formula (I-I-D-P-3-4-c-2-b):

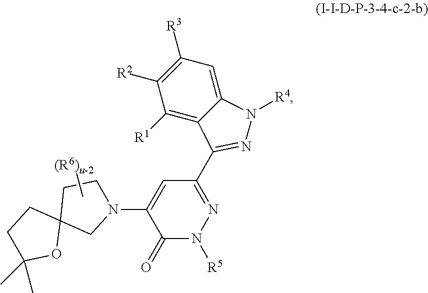

or a pharmaceutically acceptable salt, stereoisomer, solvate, prodrug, isotopic derivative, or tautomer thereof.

In some embodiments, the compound is of Formula (I-I-D-P-3-4-c-2-b-H):

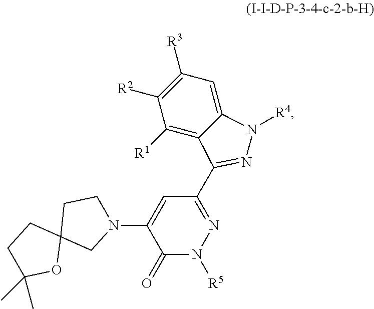

or a pharmaceutically acceptable salt, stereoisomer, solvate, prodrug, isotopic derivative, or tautomer thereof.

In some embodiments, the compound is of Formula (I-I-D-P-3-4-d):

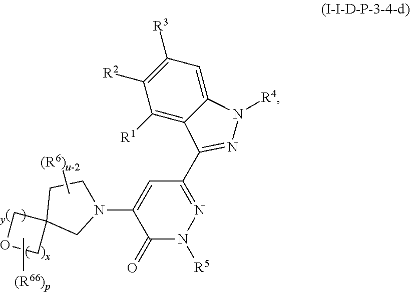

or a pharmaceutically acceptable salt, stereoisomer, solvate, prodrug, isotopic derivative, or tautomer thereof, wherein x and y are integers independently selected from 1, 2, and 3; p is an integer selected from 0, 1, and 2; R⁶⁶ is selected from halogen, OH, CN, C₁-C₆ alkyl, C₁-C₆ alkoxy, C₁-C₆ hydroxyalkyl, C₁-C₆ alkyl-C₁-C₆ alkoxy, S(O)₂R¹²; and all other variables are as described herein.

In some embodiments, the compound is of Formula (I-I-D-P-3-4-d-H):

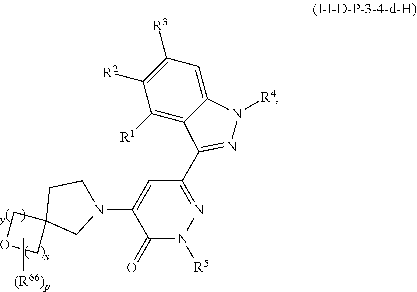

or a pharmaceutically acceptable salt, stereoisomer, solvate, prodrug, isotopic derivative, or tautomer thereof, wherein x and y are integers independently selected from 1, 2, and 3; p is an integer selected from 0, 1, and 2; R⁶⁶ is selected from halogen, OH, CN, C₁-C₆ alkyl, C₁-C₆ alkoxy, C₁-C₆ hydroxyalkyl, C₁-C₆ alkyl-C₁-C₆ alkoxy, S(O)₂R¹²; and all other variables are as described herein.

In some embodiments, the compound is of Formula (I-I-D-P-3-4-d-1):

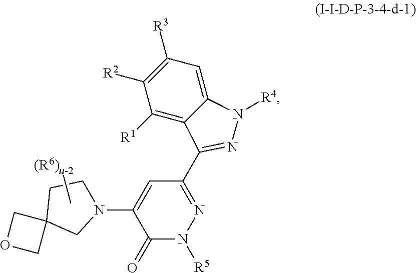

or a pharmaceutically acceptable salt, stereoisomer, solvate, prodrug, isotopic derivative, or tautomer thereof.

In some embodiments, the compound is of Formula (I-I-D-P-3-4-d-1-H):

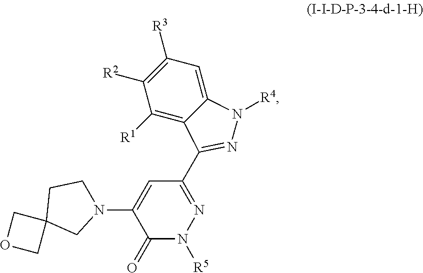

or a pharmaceutically acceptable salt, stereoisomer, solvate, prodrug, isotopic derivative, or tautomer thereof.

In some embodiments, the compound is of Formula (I-I-D-P-3-4-d-2):

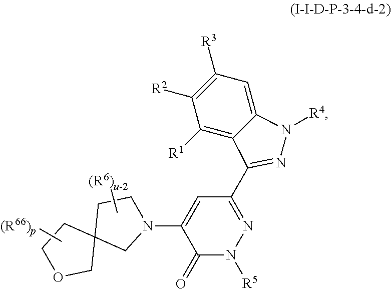

or a pharmaceutically acceptable salt, stereoisomer, solvate, prodrug, isotopic derivative, or tautomer thereof, wherein p is an integer selected from 0, 1, and 2; R⁶⁶ is selected from halogen, OH, CN, C₁-C₆ alkyl, C₁-C₆ alkoxy, C₁-C₆ hydroxyalkyl, C₁-C₆ alkyl-C₁-C₆ alkoxy, S(O)₂R¹²; and all other variables are as described herein.

In some embodiments, the compound is of Formula (I-I-D-P-3-4-d-2-H):

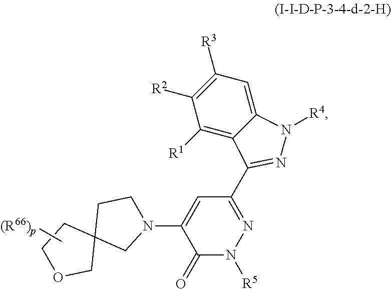

or a pharmaceutically acceptable salt, stereoisomer, solvate, prodrug, isotopic derivative, or tautomer thereof, wherein p is an integer selected from 0, 1, and 2; R⁶⁶ is selected from halogen, OH, CN, C₁-C₆ alkyl, C₁-C₆ alkoxy, C₁-C₆ hydroxyalkyl, C₁-C₆ alkyl-C₁-C₆ alkoxy, S(O)₂R¹²; and all other variables are as described herein.

In some embodiments, the compound is of Formula (I-I-D-P-3-4-d-2-a):

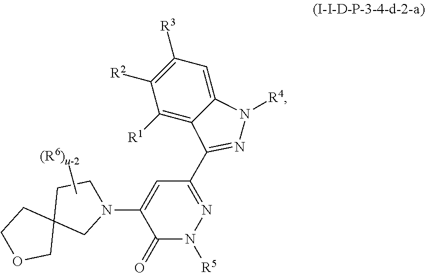

or a pharmaceutically acceptable salt, stereoisomer, solvate, prodrug, isotopic derivative, or tautomer thereof.

In some embodiments, the compound is of Formula (I-I-D-P-3-4-d-2-a-H):

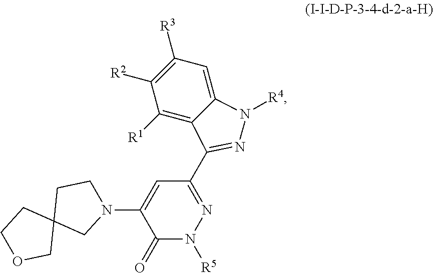

or a pharmaceutically acceptable salt, stereoisomer, solvate, prodrug, isotopic derivative, or tautomer thereof.

In some embodiments, the compound is of Formula (I-I-D-P-3-4-d-2-b):

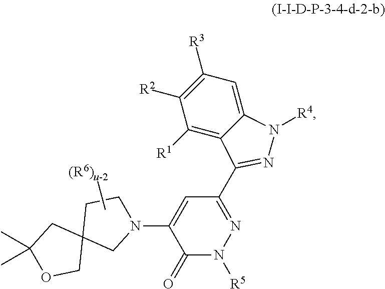

or a pharmaceutically acceptable salt, stereoisomer, solvate, prodrug, isotopic derivative, or tautomer thereof.

In some embodiments, the compound is of Formula (I-I-D-P-3-4-d-2-b-H):

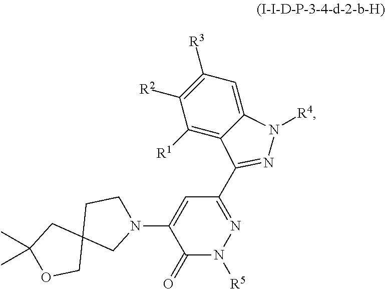

or a pharmaceutically acceptable salt, stereoisomer, solvate, prodrug, isotopic derivative, or tautomer thereof.

In some embodiments, the compound is of Formula (I-I-D-P-3-4-e):

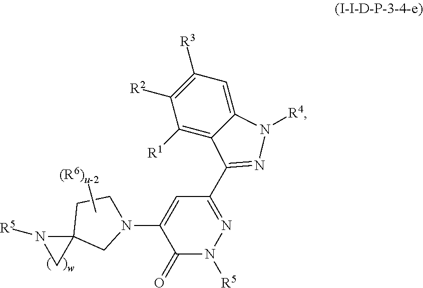

or a pharmaceutically acceptable salt, stereoisomer, solvate, prodrug, isotopic derivative, or tautomer thereof, wherein w is an integer selected from 1, 2, 3, 4 and 5; and all other variables are as described herein.

In some embodiments, the compound is of Formula (I-I-D-P-3-4-e-H):

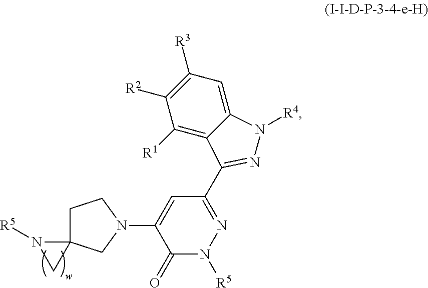

or a pharmaceutically acceptable salt, stereoisomer, solvate, prodrug, isotopic derivative, or tautomer thereof, wherein w is an integer selected from 1, 2, 3, 4 and 5; and all other variables are as described herein.

In some embodiments, the compound is of Formula (I-I-D-P-3-4-e-1):

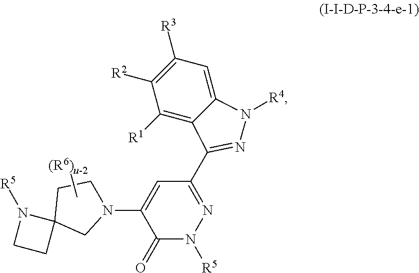

or a pharmaceutically acceptable salt, stereoisomer, solvate, prodrug, isotopic derivative, or tautomer thereof.

In some embodiments, the compound is of Formula (I-I-D-P-3-4-e-1-H):

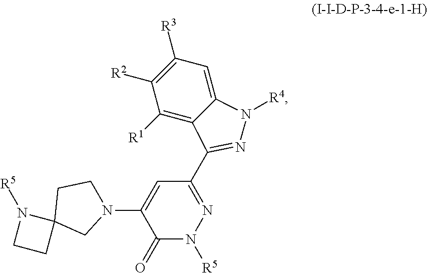

or a pharmaceutically acceptable salt, stereoisomer, solvate, prodrug, isotopic derivative, or tautomer thereof.

In some embodiments, the compound is of Formula (I-I-D-P-3-4-e-1-a):

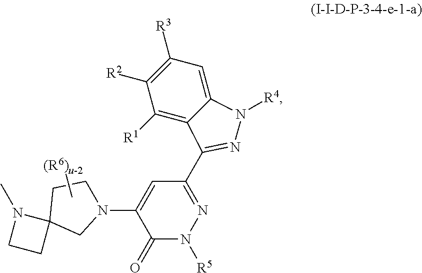

or a pharmaceutically acceptable salt, stereoisomer, solvate, prodrug, isotopic derivative, or tautomer thereof.

In some embodiments, the compound is of Formula (I-I-D-P-3-4-e-1-a-H):

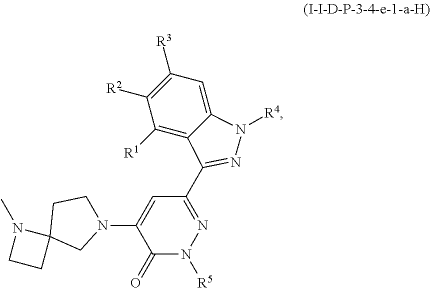

or a pharmaceutically acceptable salt, stereoisomer, solvate, prodrug, isotopic derivative, or tautomer thereof.

In some embodiments, the compound is of Formula (I-I-D-P-3-4-e-2):

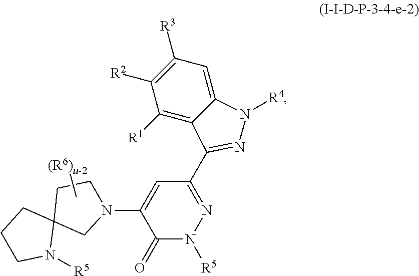

or a pharmaceutically acceptable salt, stereoisomer, solvate, prodrug, isotopic derivative, or tautomer thereof.

In some embodiments, the compound is of Formula (I-I-D-P-3-4-e-2-H):

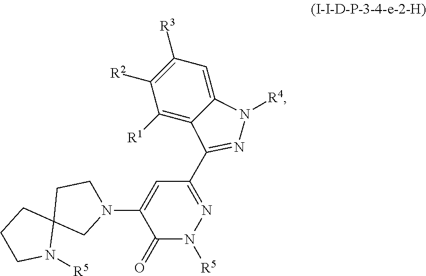

or a pharmaceutically acceptable salt, stereoisomer, solvate, prodrug, isotopic derivative, or tautomer thereof.

In some embodiments, the compound is of Formula (I-I-D-P-3-4-e-2-a):

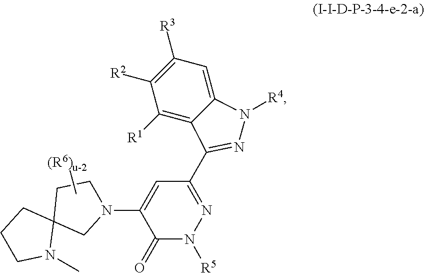

or a pharmaceutically acceptable salt, stereoisomer, solvate, prodrug, isotopic derivative, or tautomer thereof.

In some embodiments, the compound is of Formula (I-I-D-P-3-4-e-2-a-H):

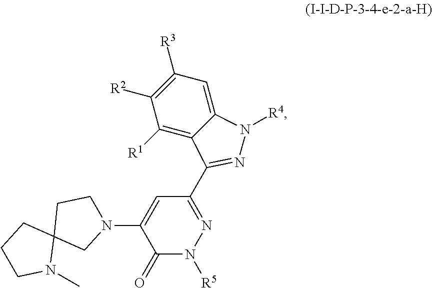

or a pharmaceutically acceptable salt, stereoisomer, solvate, prodrug, isotopic derivative, or tautomer thereof.

In some embodiments, the compound is of Formula (I-I-D-P-3-4-e-2-b):

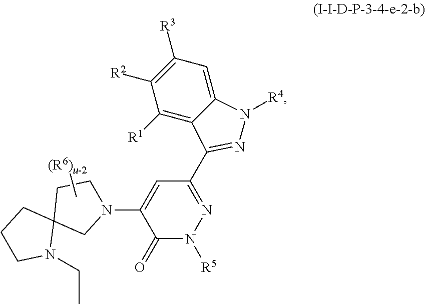

or a pharmaceutically acceptable salt, stereoisomer, solvate, prodrug, isotopic derivative, or tautomer thereof.

In some embodiments, the compound is of Formula (I-I-D-P-3-4-e-2-b-H):

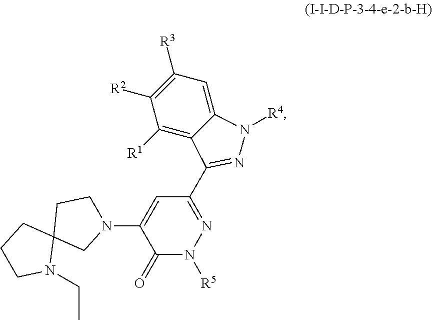

or a pharmaceutically acceptable salt, stereoisomer, solvate, prodrug, isotopic derivative, or tautomer thereof.

In some embodiments, the compound is of Formula (I-I-D-P-3-4-f):

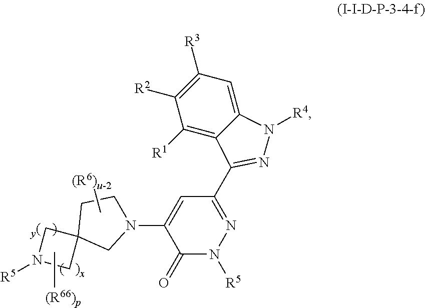

or a pharmaceutically acceptable salt, stereoisomer, solvate, prodrug, isotopic derivative, or tautomer thereof, wherein x and y are integers independently selected from 1, 2, and 3; p is an integer selected from 0, 1, and 2; R⁶⁶ is selected from halogen, OH, CN, C₁-C₆ alkyl, C₁-C₆ alkoxy, C₁-C₆ hydroxyalkyl, C₁-C₆ alkyl-C₁-C₆ alkoxy, S(O)₂R¹²; and all other variables are as described herein.

In some embodiments, the compound is of Formula (I-I-D-P-3-4-f-H):

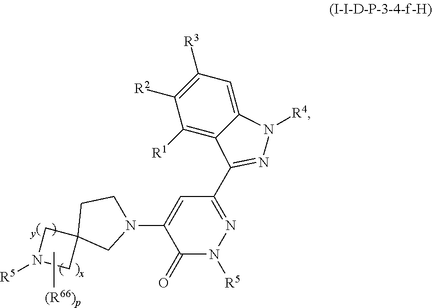

or a pharmaceutically acceptable salt, stereoisomer, solvate, prodrug, isotopic derivative, or tautomer thereof, wherein x and y are integers independently selected from 1, 2, and 3; p is an integer selected from 0, 1, and 2; R⁶⁶ is selected from halogen, OH, CN, C₁-C₆ alkyl, C₁-C₆ alkoxy, C₁-C₆ hydroxyalkyl, C₁-C₆ alkyl-C₁-C₆ alkoxy, S(O)₂R¹²; and all other variables are as described herein.

In some embodiments, the compound is of Formula (I-I-D-P-3-4-f-1):

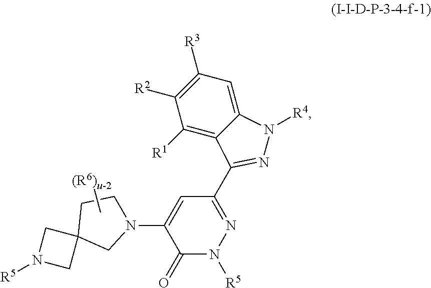

or a pharmaceutically acceptable salt, stereoisomer, solvate, prodrug, isotopic derivative, or tautomer thereof.

In some embodiments, the compound is of Formula (I-I-D-P-3-4-f-1-H):

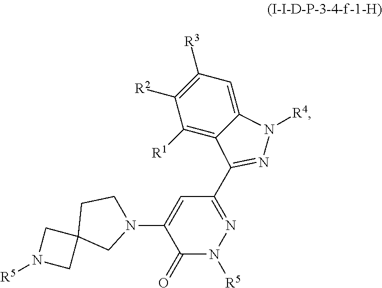

or a pharmaceutically acceptable salt, stereoisomer, solvate, prodrug, isotopic derivative, or tautomer thereof.

In some embodiments, the compound is of Formula (I-I-D-P-3-4-f-1-a):

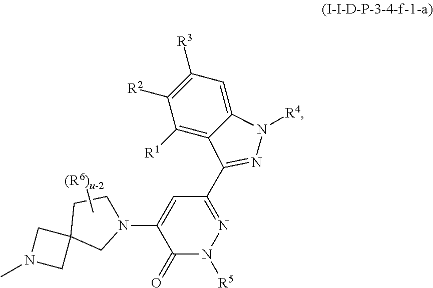

or a pharmaceutically acceptable salt, stereoisomer, solvate, prodrug, isotopic derivative, or tautomer thereof.

In some embodiments, the compound is of Formula (I-I-D-P-3-4-f-1-a-H):

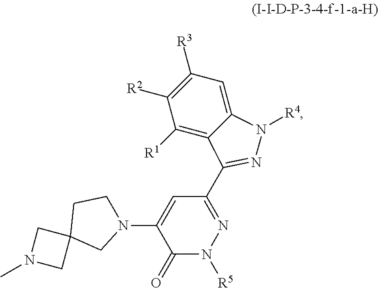

or a pharmaceutically acceptable salt, stereoisomer, solvate, prodrug, isotopic derivative, or tautomer thereof.

In some embodiments, the compound is of Formula (I-I-D-P-3-4-f-2):

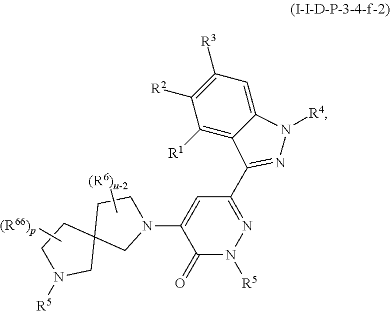

or a pharmaceutically acceptable salt, stereoisomer, solvate, prodrug, isotopic derivative, or tautomer thereof, wherein p is an integer selected from 0, 1, and 2; R⁶⁶ is selected from halogen, OH, CN, C₁-C₆ alkyl, C₁-C₆ alkoxy, C₁-C₆ hydroxyalkyl, C₁-C₆ alkyl-C₁-C₆ alkoxy, S(O)₂R¹²; and all other variables are as described herein.

In some embodiments, the compound is of Formula (I-I-D-P-3-4-f-2-H):

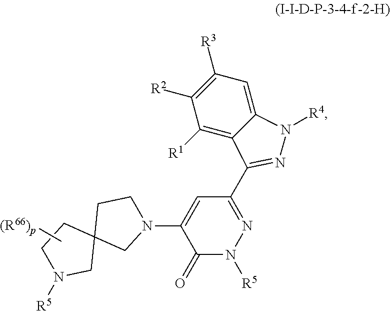

or a pharmaceutically acceptable salt, stereoisomer, solvate, prodrug, isotopic derivative, or tautomer thereof, wherein p is an integer selected from 0, 1, and 2; R⁶⁶ is selected from halogen, OH, CN, C₁-C₆ alkyl, C₁-C₆ alkoxy, C₁-C₆ hydroxyalkyl, C₁-C₆ alkyl-C₁-C₆ alkoxy, S(O)₂R¹²; and all other variables are as described herein.

In some embodiments, the compound is of Formula (I-I-D-P-3-4-f-2-a):

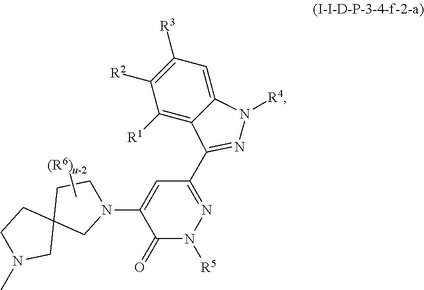

or a pharmaceutically acceptable salt, stereoisomer, solvate, prodrug, isotopic derivative, or tautomer thereof.

In some embodiments, the compound is of Formula (I-I-D-P-3-4-f-2-a-H):

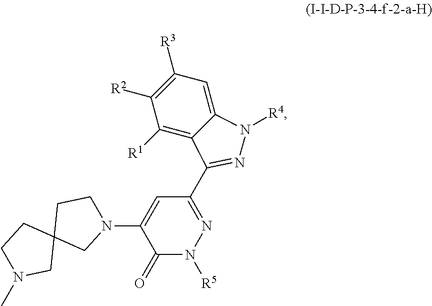

or a pharmaceutically acceptable salt, stereoisomer, solvate, prodrug, isotopic derivative, or tautomer thereof.

In some embodiments, the compound is of Formula (I-I-D-PP):

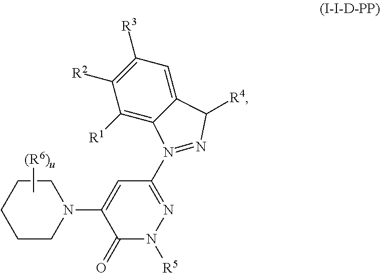

or a pharmaceutically acceptable salt, stereoisomer, solvate, prodrug, isotopic derivative, or tautomer thereof.

In some embodiments, the compound is of Formula (I-I-D-PP-H):

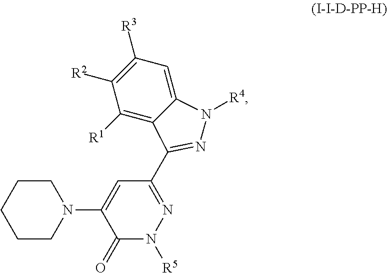

or a pharmaceutically acceptable salt, stereoisomer, solvate, prodrug, isotopic derivative, or tautomer thereof.

In some embodiments, the compound is of Formula (I-I-D-PP-1):

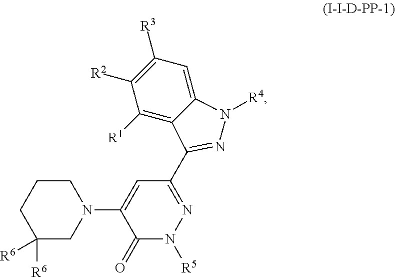

or a pharmaceutically acceptable salt, stereoisomer, solvate, prodrug, isotopic derivative, or tautomer thereof.

In some embodiments, the compound is of Formula (I-I-D-PP-1-a):

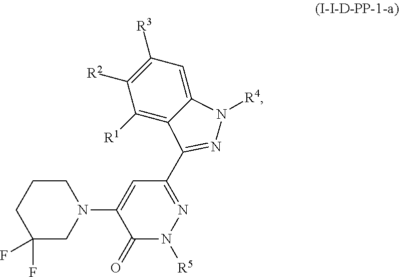

or a pharmaceutically acceptable salt, stereoisomer, solvate, prodrug, isotopic derivative, or tautomer thereof.

In some embodiments, the compound is of Formula (I-I-D-A):

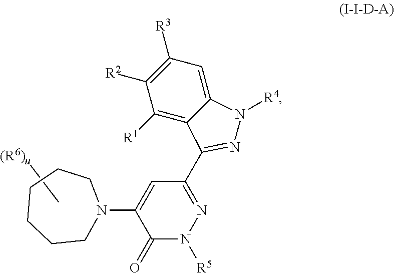

or a pharmaceutically acceptable salt, stereoisomer, solvate, prodrug, isotopic derivative, or tautomer thereof.

In some embodiments, the compound is of Formula (I-I-D-A-H):

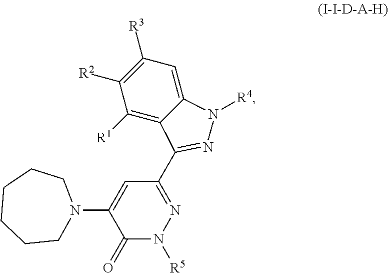

or a pharmaceutically acceptable salt, stereoisomer, solvate, prodrug, isotopic derivative, or tautomer thereof.

In some embodiments, the compound is of Formula (I-I-D-A-1):

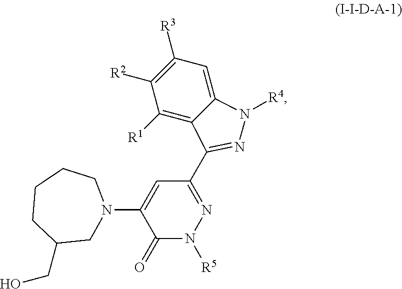

or a pharmaceutically acceptable salt, stereoisomer, solvate, prodrug, isotopic derivative, or tautomer thereof.

In some embodiments, the compound is of Formula (I-I-D-A-2):

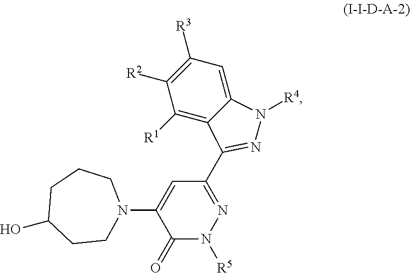

or a pharmaceutically acceptable salt, stereoisomer, solvate, prodrug, isotopic derivative, or tautomer thereof.

In some embodiments, the compound is of Formula (I-I-D-A-3):

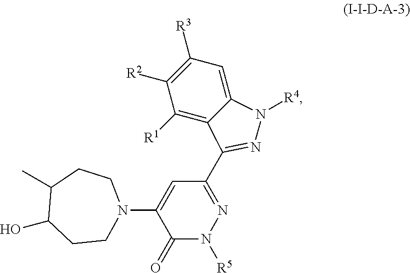

or a pharmaceutically acceptable salt, stereoisomer, solvate, prodrug, isotopic derivative, or tautomer thereof.

In some embodiments, the compound is of Formula (I-I-D-A-3*):

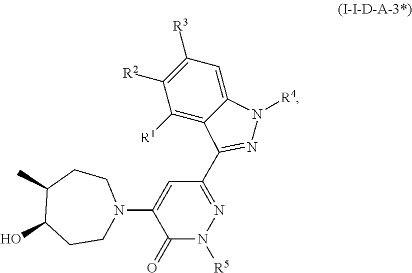

or a pharmaceutically acceptable salt, stereoisomer, solvate, prodrug, isotopic derivative, or tautomer thereof.

In some embodiments, the compound is of Formula (I-I-D-A-3**):

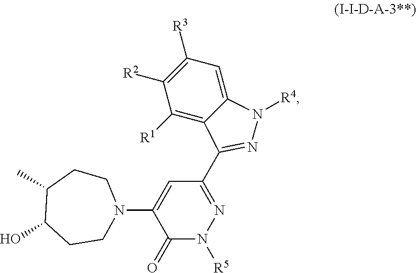

or a pharmaceutically acceptable salt, stereoisomer, solvate, prodrug, isotopic derivative, or tautomer thereof.

In some embodiments, the compound is of Formula (I-I-D-A-3***):

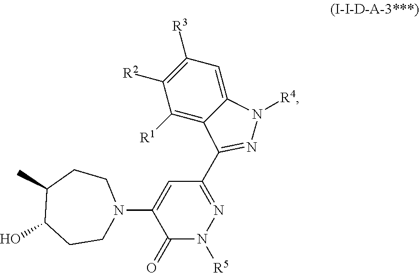

or a pharmaceutically acceptable salt, stereoisomer, solvate, prodrug, isotopic derivative, or tautomer thereof.

In some embodiments, the compound is of Formula (I-I-D-A-3****):

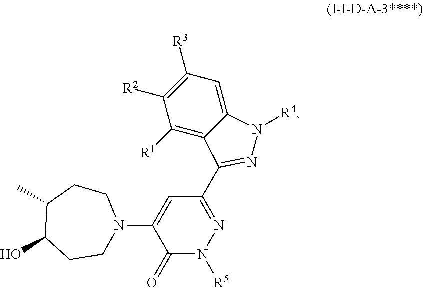

or a pharmaceutically acceptable salt, stereoisomer, solvate, prodrug, isotopic derivative, or tautomer thereof.

In some embodiments, the compound is of Formula (I-I-D-A-4):

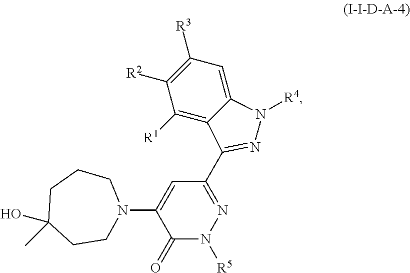

or a pharmaceutically acceptable salt, stereoisomer, solvate, prodrug, isotopic derivative, or tautomer thereof.

In some embodiments, the compound is of Formula (I-I-D-A-4*):

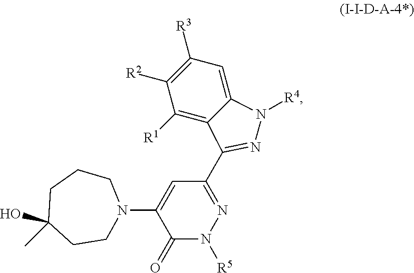

or a pharmaceutically acceptable salt, stereoisomer, solvate, pro rug, isotopic derivative, or tautomer thereof.

In some embodiments, the compound is of Formula (I-I-D-A-4**):

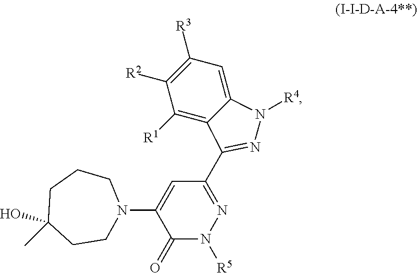

or a pharmaceutically acceptable salt, stereoisomer, solvate, prodrug, isotopic derivative, or tautomer thereof.

In some embodiments, the compound is of Formula (I-I-D-A-5):

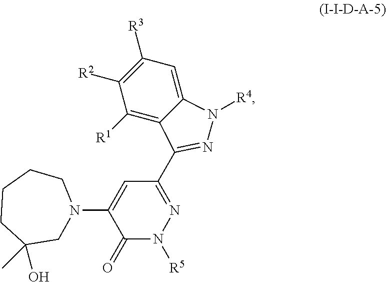

or a pharmaceutically acceptable salt, stereoisomer, solvate, prodrug, isotopic derivative, or tautomer thereof.

In some embodiments, the compound is of Formula (I-I-D-A-6):

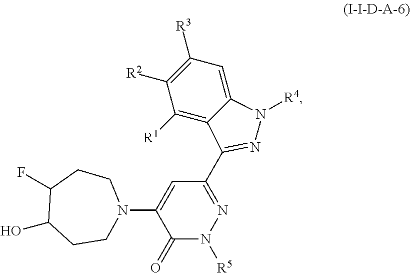

or a pharmaceutically acceptable salt, stereoisomer, solvate, prodrug, isotopic derivative, or tautomer thereof.

In some embodiments, the compound is of Formula (I-I-D-A-6*):

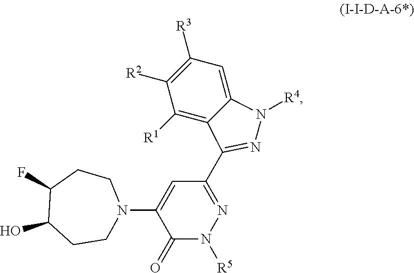

or a pharmaceutically acceptable salt, stereoisomer, solvate, prodrug, isotopic derivative, or tautomer thereof.

In some embodiments, the compound is of Formula (I-I-D-A-6**):

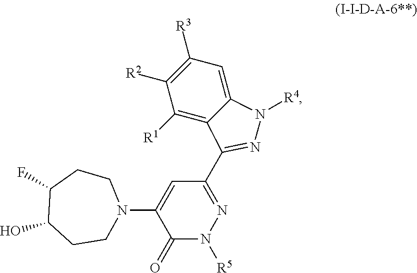

or a pharmaceutically acceptable salt, stereoisomer, solvate, prodrug, isotopic derivative, or tautomer thereof.

In some embodiments, the compound is of Formula (I-I-D-A-6***):

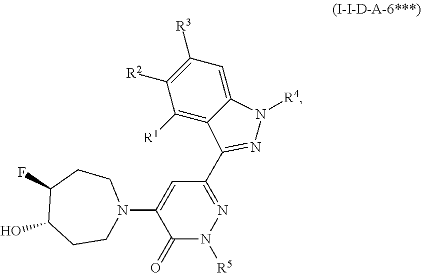

or a pharmaceutically acceptable salt, stereoisomer, solvate, prodrug, isotopic derivative, or tautomer thereof.

In some embodiments, the compound is of Formula (I-I-D-A-6****):

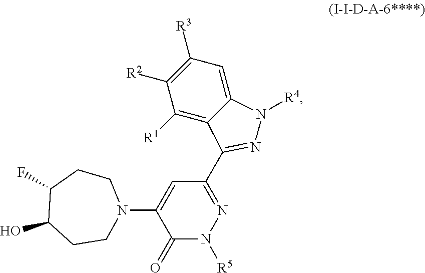

or a pharmaceutically acceptable salt, stereoisomer, solvate, prodrug, isotopic derivative, or tautomer thereof.

In some embodiments, the compound is of Formula (I-I-D-A-7):

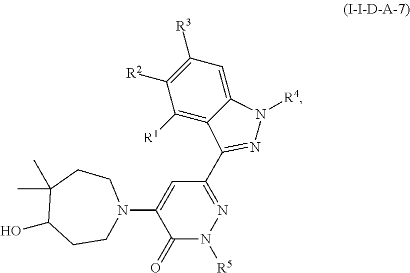

or a pharmaceutically acceptable salt, stereoisomer, solvate, prodrug, isotopic derivative, or tautomer thereof.

In some embodiments, the compound is of Formula (I-I-D-A-8):

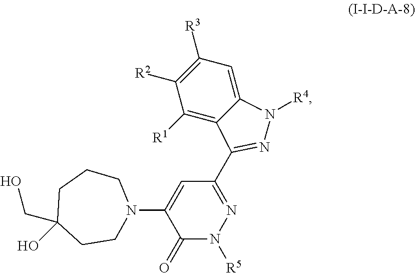

or a pharmaceutically acceptable salt, stereoisomer, solvate, prodrug, isotopic derivative, or tautomer thereof.

In some embodiments, the compound is of Formula (I-I-D-IV):

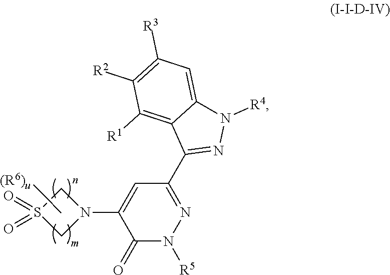

or a pharmaceutically acceptable salt, stereoisomer, solvate, prodrug, isotopic derivative, or tautomer thereof.

In some embodiments, the compound is of Formula (I-I-D-T):

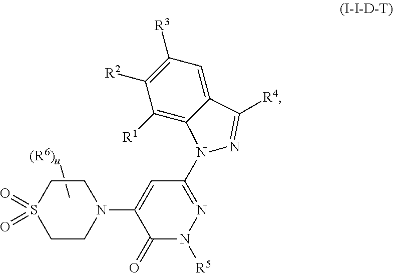

or a pharmaceutically acceptable salt, stereoisomer, solvate, prodrug, isotopic derivative, or tautomer thereof.

In some embodiments, the compound is of Formula (I-I-D-T-H):

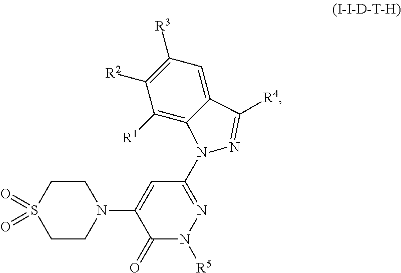

or a pharmaceutically acceptable salt, stereoisomer, solvate, prodrug, isotopic derivative, or tautomer thereof.

In some embodiments, the compound is of Formula (I-I-D-TZ):

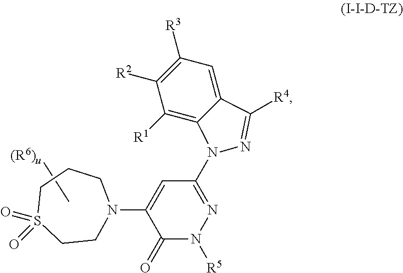

or a pharmaceutically acceptable salt, stereoisomer, solvate, prodrug, isotopic derivative, or tautomer thereof.

In some embodiments, the compound is of Formula (I-I-D-TZ-H):

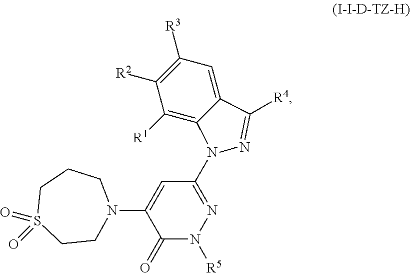

or a pharmaceutically acceptable salt, stereoisomer, solvate, prodrug, isotopic derivative, or tautomer thereof.

In some embodiments, the compound is of Formula (I-II-A-M):

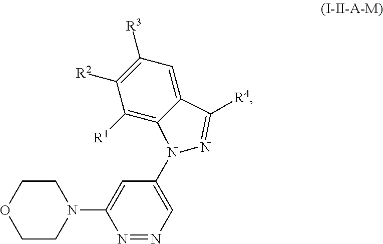

or a pharmaceutically acceptable salt, stereoisomer, solvate, prodrug, isotopic derivative, or tautomer thereof.

In some embodiments, the compound is of Formula (I-II-B-M):

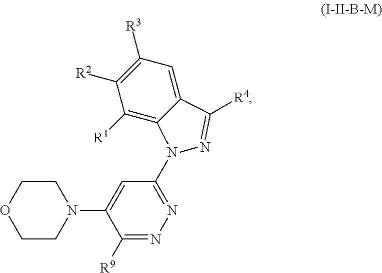

or a pharmaceutically acceptable salt, stereoisomer, solvate, prodrug, isotopic derivative, or tautomer thereof.

In some embodiments, the compound is of Formula (I-II-C-M):

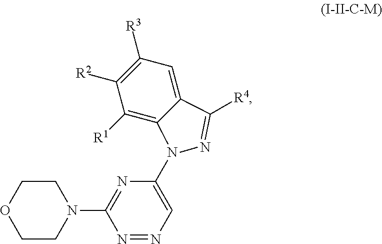

or a pharmaceutically acceptable salt, stereoisomer, solvate, prodrug, isotopic derivative, or tautomer thereof.

In some embodiments, the compound is of Formula (I-II-D-M):

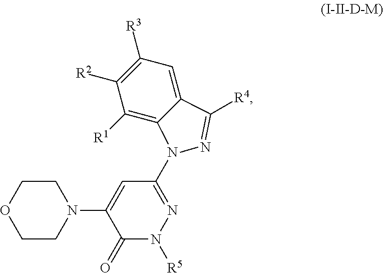

or a pharmaceutically acceptable salt, stereoisomer, solvate, prodrug, isotopic derivative, or tautomer thereof.

In some embodiments, the compound is of Formula (I-I-A-P):

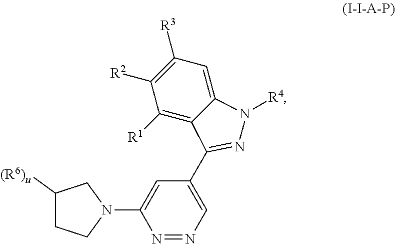

or a pharmaceutically acceptable salt, stereoisomer, solvate, prodrug, isotopic derivative, or tautomer thereof wherein u is 0 or 1 and all other variables are as defined herein.

In some embodiments, the compound is of Formula (I-I-B-P):

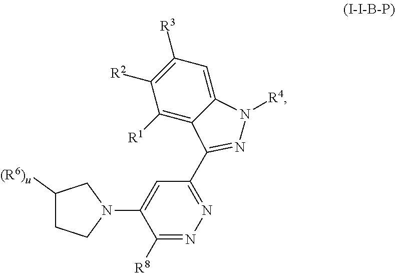

or a pharmaceutically acceptable salt, stereoisomer, solvate, prodrug, isotopic derivative, or tautomer thereof wherein u is 0 or 1 and all other variables are as defined herein.

In some embodiments, the compound is of Formula (I-I-C-P):

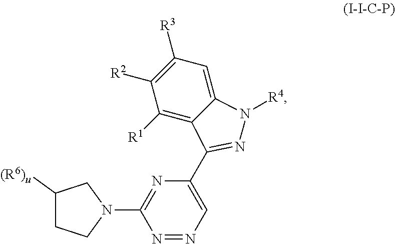

or a pharmaceutically acceptable salt, stereoisomer, solvate, prodrug, isotopic derivative, or tautomer thereof wherein u is 0 or 1 and all other variables are as defined herein.

In some embodiments, the compound is of Formula (I-I-D-P):

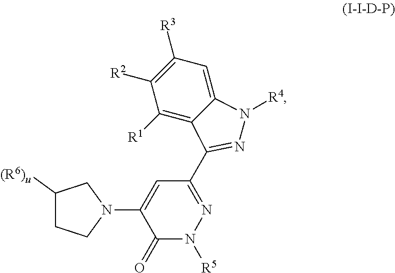

or a pharmaceutically acceptable salt, stereoisomer, solvate, prodrug, isotopic derivative, or tautomer thereof wherein u is 0 or 1 and all other variables are as defined herein.

In some embodiments, the compound is of Formula (I-II-A-P):

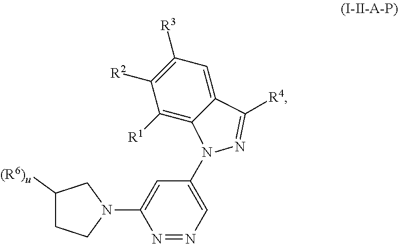

or a pharmaceutically acceptable salt, stereoisomer, solvate, prodrug, isotopic derivative, or tautomer thereof wherein u is 0 or 1 and all other variables are as defined herein.

In some embodiments, the compound is of Formula (I-II-B-P):

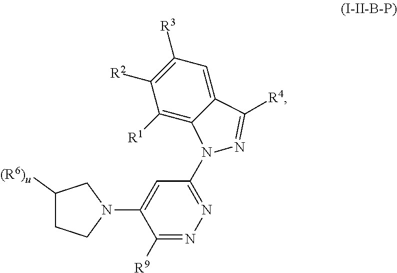

or a pharmaceutically acceptable salt, stereoisomer, solvate, prodrug, isotopic derivative, or tautomer thereof wherein u is 0 or 1 and all other variables are as defined herein.

In some embodiments, the compound is of Formula (I-II-C-P):

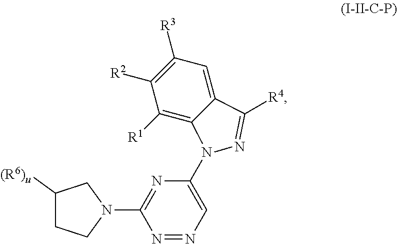

or a pharmaceutically acceptable salt, stereoisomer, solvate, prodrug, isotopic derivative, or tautomer thereof wherein u is 0 or 1 and all other variables are as defined herein.

In some embodiments, the compound is of Formula (I-II-D-P):

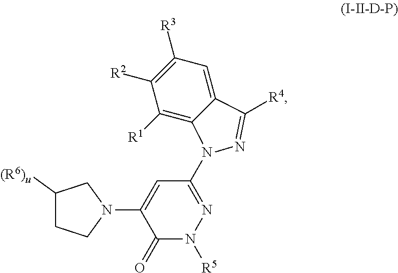

or a pharmaceutically acceptable salt, stereoisomer, solvate, prodrug, isotopic derivative, or tautomer thereof wherein u is 0 or 1 and all other variables are as defined herein.

In some embodiments, the compound is of Formula (I-I-A-A):

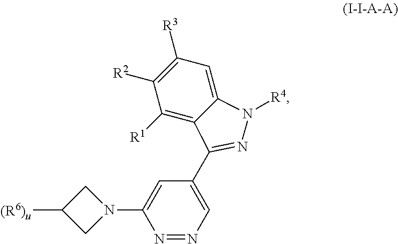

or a pharmaceutically acceptable salt, stereoisomer, solvate, prodrug, isotopic derivative, or tautomer thereof wherein u is 0 or 1 and all other variables are as defined herein.

In some embodiments, the compound is of Formula (I-I-B-A):

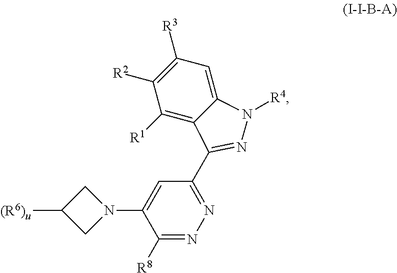

or a pharmaceutically acceptable salt, stereoisomer, solvate, prodrug, isotopic derivative, or tautomer thereof wherein u is 0 or 1 and all other variables are as defined herein.

In some embodiments, the compound is of Formula (I-I-C-A):

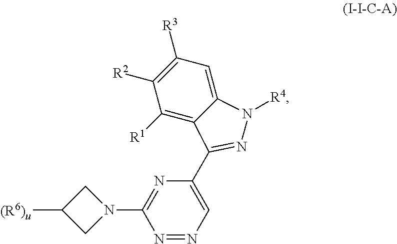

or a pharmaceutically acceptable salt, stereoisomer, solvate, prodrug, isotopic derivative, or tautomer thereof wherein u is 0 or 1 and all other variables are as defined herein.

In some embodiments, the compound is of Formula (I-I-D-A):

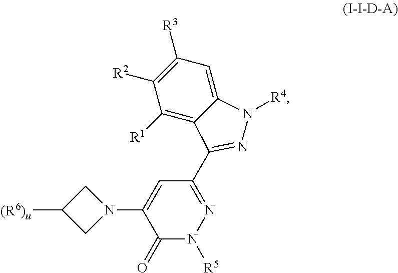

or a pharmaceutically acceptable salt, stereoisomer, solvate, prodrug, isotopic derivative, or tautomer thereof wherein u is 0 or 1 and all other variables are as defined herein.

In some embodiments, the compound is of Formula (I-II-A-A):

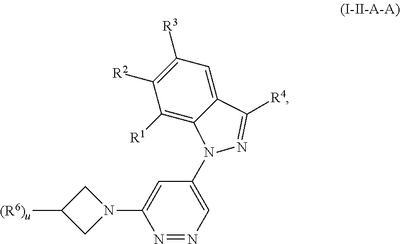

or a pharmaceutically acceptable salt, stereoisomer, solvate, prodrug, isotopic derivative, or tautomer thereof wherein u is 0 or 1 and all other variables are as defined herein.

In some embodiments, the compound is of Formula (I-II-B-A):

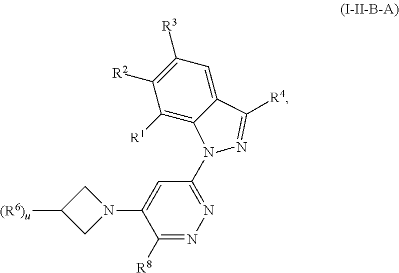

or a pharmaceutically acceptable salt, stereoisomer, solvate, prodrug, isotopic derivative, or tautomer thereof wherein u is 0 or 1 and all other variables are as defined herein.

In some embodiments, the compound is of Formula (I-II-C-A):

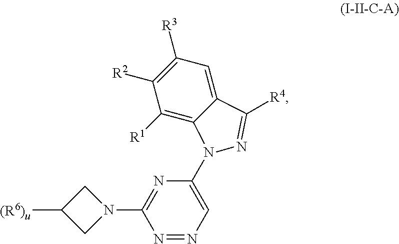

or a pharmaceutically acceptable salt, stereoisomer, solvate, prodrug, isotopic derivative, or tautomer thereof wherein u is 0 or 1 and all other variables are as defined herein.

In some embodiments, the compound is of Formula (I-II-D-A):

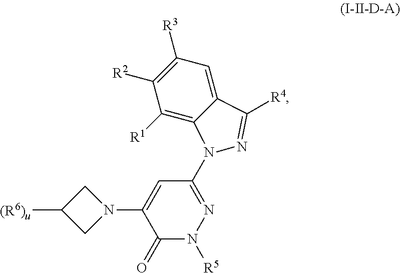

or a pharmaceutically acceptable salt, stereoisomer, solvate, prodrug, isotopic derivative, or tautomer thereof wherein u is 0 or 1 and all other variables are as defined herein.

In some embodiments, the compound is of Formula (I-I-A-1):

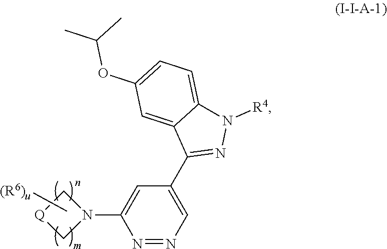

or a pharmaceutically acceptable salt, stereoisomer, solvate, prodrug, isotopic derivative, or tautomer thereof.

In some embodiments, the compound is of Formula (I-I-A-1-H):

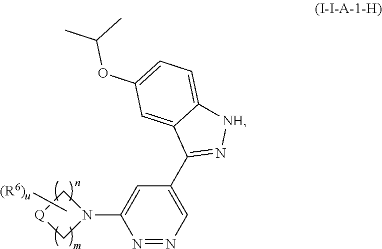

or a pharmaceutically acceptable salt, stereoisomer, solvate, prodrug, isotopic derivative, or tautomer thereof.

In some embodiments, the compound is of Formula (I-I-A-2):

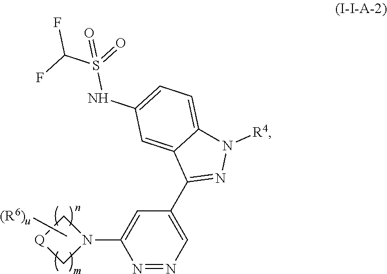

or a pharmaceutically acceptable salt, stereoisomer, solvate, prodrug, isotopic derivative, or tautomer thereof.

In some embodiments, the compound is of Formula (I-I-A-2-H):

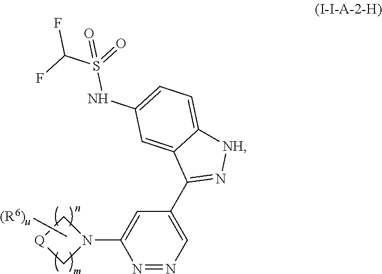

or a pharmaceutically acceptable salt, stereoisomer, solvate, prodrug, isotopic derivative, or tautomer thereof.

In some embodiments, the compound is of Formula (I-I-A-3):

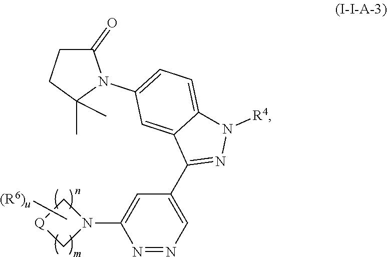

or a pharmaceutically acceptable salt, stereoisomer, solvate, prodrug, isotopic derivative, or tautomer thereof.

In some embodiments, the compound is of Formula (I-I-A-3-H):

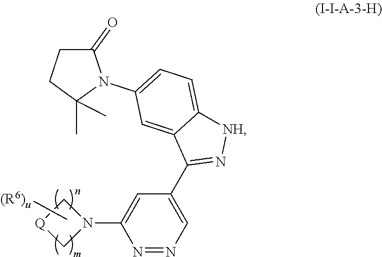

or a pharmaceutically acceptable salt, stereoisomer, solvate, prodrug, isotopic derivative, or tautomer thereof.

In some embodiments, the compound is of Formula (I-I-A-4):

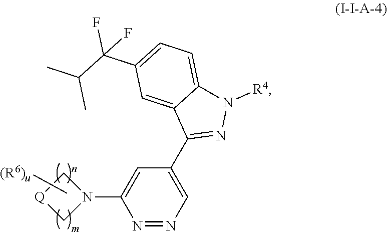

or a pharmaceutically acceptable salt, stereoisomer, solvate, prodrug, isotopic derivative, or tautomer thereof.

In some embodiments, the compound is of Formula (I-I-A-4-H):

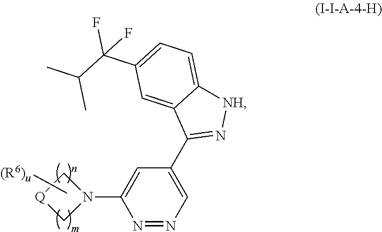

or a pharmaceutically acceptable salt, stereoisomer, solvate, prodrug, isotopic derivative, or tautomer thereof.

In some embodiments, the compound is of Formula (I-I-A-5):

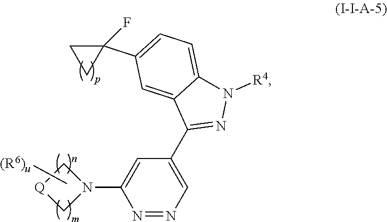

or a pharmaceutically acceptable salt, stereoisomer, solvate, prodrug, isotopic derivative, or tautomer thereof, wherein p is integer selected from 1, 2, 3, and 4 and all other variables are as defined herein.

In some embodiments, the compound is of Formula (I-I-A-5-H):

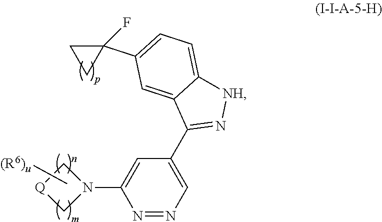

or a pharmaceutically acceptable salt, stereoisomer, solvate, prodrug, isotopic derivative, or tautomer thereof, wherein p is integer selected from 1, 2, 3, and 4 and all other variables are as defined herein.

In some embodiments, the compound is of Formula (I-I-B-1):

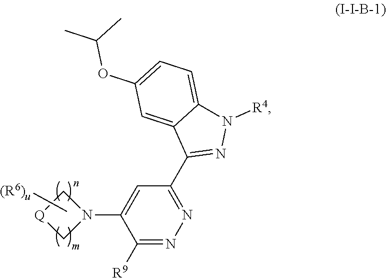

or a pharmaceutically acceptable salt, stereoisomer, solvate, prodrug, isotopic derivative, or tautomer thereof.

In some embodiments, the compound is of Formula (I-I-B-1-H):

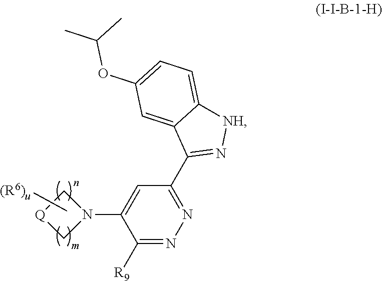

or a pharmaceutically acceptable salt, stereoisomer, solvate, prodrug, isotopic derivative, or tautomer thereof.

In some embodiments, the compound is of Formula (I-I-C-1):

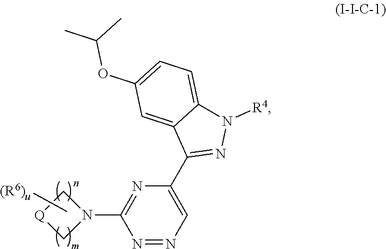

or a pharmaceutically acceptable salt, stereoisomer, solvate, prodrug, isotopic derivative, or tautomer thereof.

In some embodiments, the compound is of Formula (I-I-C-1-H):

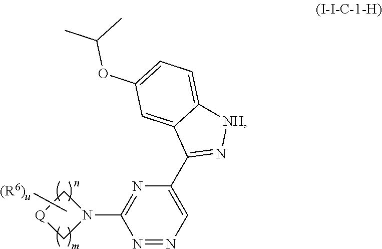

or a pharmaceutically acceptable salt, stereoisomer, solvate, prodrug, isotopic derivative, or tautomer thereof.

In some embodiments, the compound is of Formula (I-I-D-1):

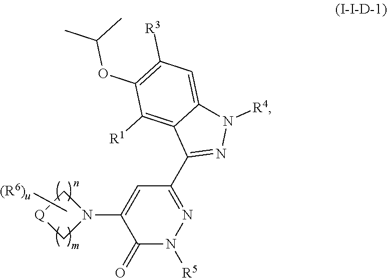

or a pharmaceutically acceptable salt, stereoisomer, solvate, prodrug, isotopic derivative, or tautomer thereof.

In some embodiments, the compound is of Formula (I-I-D-1-H):

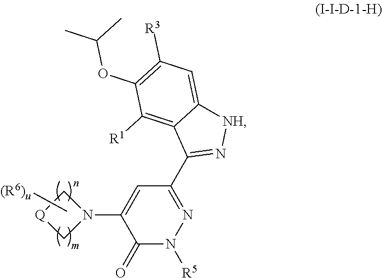

or a pharmaceutically acceptable salt, stereoisomer, solvate, prodrug, isotopic derivative, or tautomer thereof.

In some embodiments, the compound is of Formula (I-I-D-1-H-1):

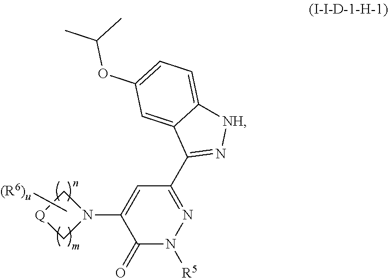

or a pharmaceutically acceptable salt, stereoisomer, solvate, prodrug, isotopic derivative, or tautomer thereof.

In some embodiments, the compound is of Formula (I-I-D-1-H-2):

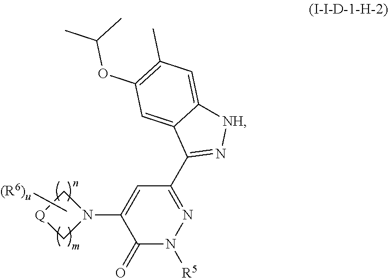

or a pharmaceutically acceptable salt, stereoisomer, solvate, prodrug, isotopic derivative, or tautomer thereof.

In some embodiments, the compound is of Formula (I-I-D-1-H-3):

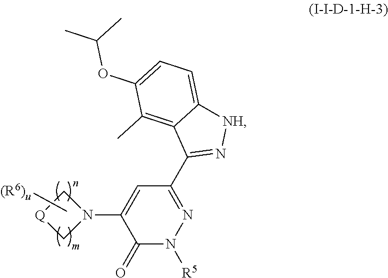

or a pharmaceutically acceptable salt, stereoisomer, solvate, prodrug, isotopic derivative, or tautomer thereof.

In some embodiments, the compound is of Formula (I-I-D-2):

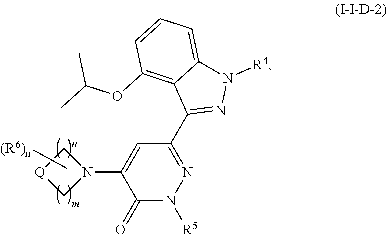

or a pharmaceutically acceptable salt, stereoisomer, solvate, prodrug, isotopic derivative, or tautomer thereof.

In some embodiments, the compound is of Formula (I-I-D-2-H):

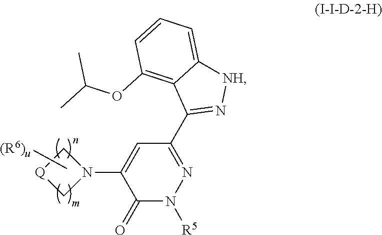

or a pharmaceutically acceptable salt, stereoisomer, solvate, prodrug, isotopic derivative, or tautomer thereof.

In some embodiments, the compound is of Formula (I-I-D-3):

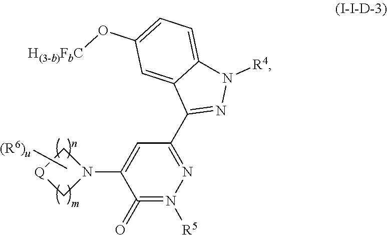

or a pharmaceutically acceptable salt, stereoisomer, solvate, prodrug, isotopic derivative, or tautomer thereof, wherein b is an integer selected from 1, 2, and 3 and all other integers are as defined herein.

In some embodiments, the compound is of Formula (I-I-D-3-H):

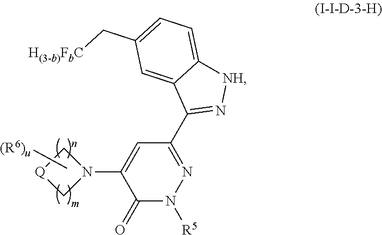

or a pharmaceutically acceptable salt, stereoisomer, solvate, prodrug, isotopic derivative, or tautomer thereof, wherein b is an integer selected from 1, 2, and 3 and all other variables are as defined herein.

In some embodiments, the compound is of Formula (I-I-D-3-1):

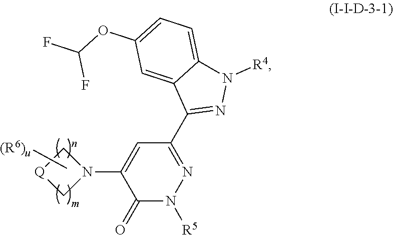

or a pharmaceutically acceptable salt, stereoisomer, solvate, prodrug, isotopic derivative, or tautomer thereof.

In some embodiments, the compound is of Formula (I-I-D-3-1-H):

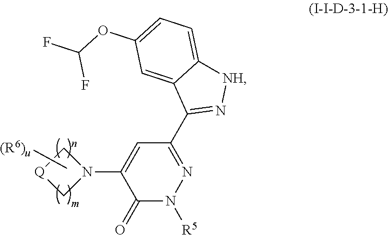

or a pharmaceutically acceptable salt, stereoisomer, solvate, prodrug, isotopic derivative, or tautomer thereof.

In some embodiments, the compound is of Formula (I-I-D-3-2):

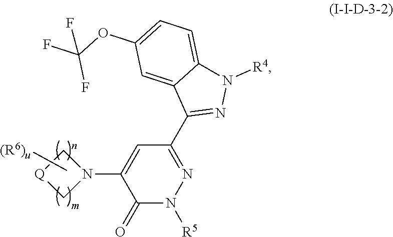

or a pharmaceutically acceptable salt, stereoisomer, solvate, prodrug, isotopic derivative, or tautomer thereof.

In some embodiments, the compound is of Formula (I-I-D-3-2-H):

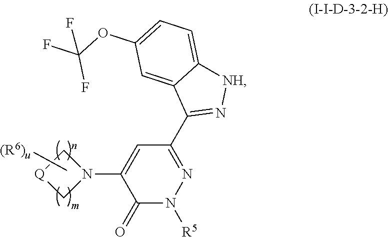

or a pharmaceutically acceptable salt, stereoisomer, solvate, prodrug, isotopic derivative, or tautomer thereof.

In some embodiments, the compound is of Formula (I-I-D-4):

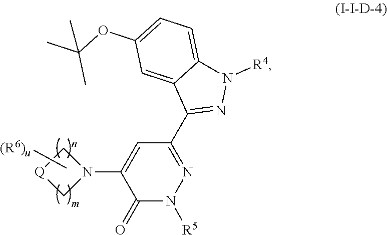

or a pharmaceutically acceptable salt, stereoisomer, solvate, prodrug, isotopic derivative, or tautomer thereof.

In some embodiments, the compound is of Formula (I-I-D-4-H):

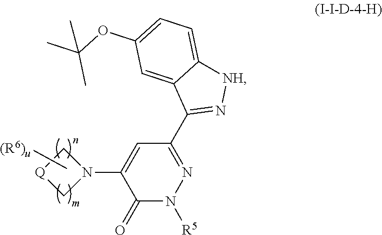

or a pharmaceutically acceptable salt, stereoisomer, solvate, prodrug, isotopic derivative, or tautomer thereof.

In some embodiments, the compound is of Formula (I-I-D-5):

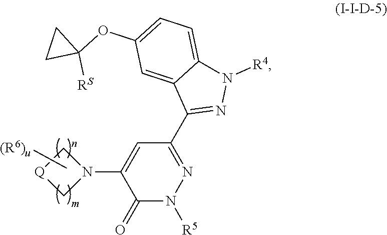

or a pharmaceutically acceptable salt, stereoisomer, solvate, prodrug, isotopic derivative, or tautomer thereof, wherein R^{s} is H, halogen, CN, or CH₃ and all other variables are as defined herein.

In some embodiments, the compound is of Formula (J-I-D-5-H):

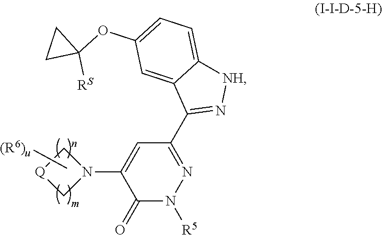

or a pharmaceutically acceptable salt, stereoisomer, solvate, prodrug, isotopic derivative, or tautomer thereof, wherein R^{s} is H, halogen, CN, or CH₃ and all other variables are as defined herein.

In some embodiments, the compound is of Formula (I-I-D-6):

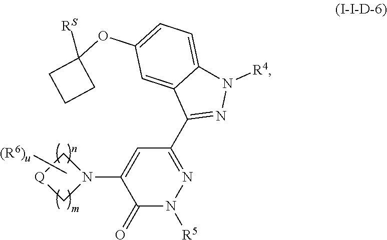

or a pharmaceutically acceptable salt, stereoisomer, solvate, prodrug, isotopic derivative, or tautomer thereof, wherein R^{s} is H, halogen, CN, or CH₃ and all other variables are as defined herein.

In some embodiments, the compound is of Formula (I-I-D-6-H):

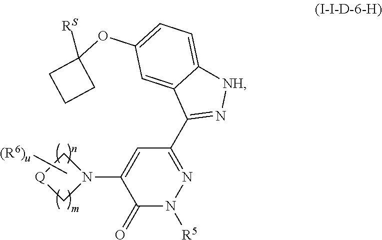

or a pharmaceutically acceptable salt, stereoisomer, solvate, prodrug, isotopic derivative, or tautomer thereof, wherein R^{s} is H, halogen, CN, or CH₃ and all other variables are as defined herein.

In some embodiments, the compound is of Formula (I-I-D-7):

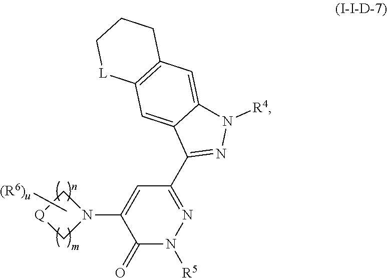

or a pharmaceutically acceptable salt, stereoisomer, solvate, prodrug, isotopic derivative, or tautomer thereof, L is CH₂, O, S, NH, and all other variables are as defined herein.

In some embodiments, the compound is of Formula (I-I-D-7-H):

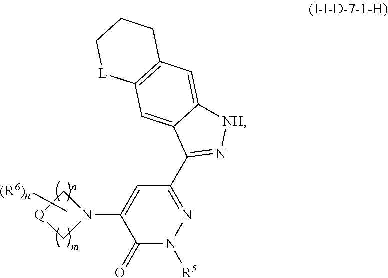

or a pharmaceutically acceptable salt, stereoisomer, solvate, prodrug, isotopic derivative, or tautomer thereof, L is CH₂, O, S, NH, and all other variables are as defined herein.

In some embodiments, the compound is of Formula (I-I-D-7-1):

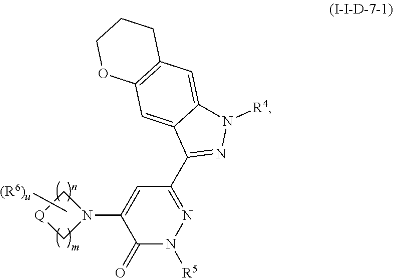

or a pharmaceutically acceptable salt, stereoisomer, solvate, prodrug, isotopic derivative, or tautomer thereof.

In some embodiments, the compound is of Formula (I-I-D-7-1-H):

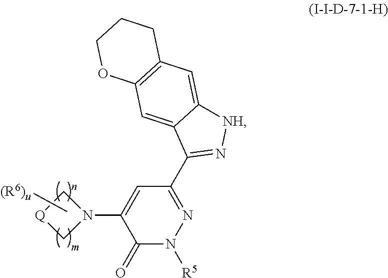

or a pharmaceutically acceptable salt, stereoisomer, solvate, prodrug, isotopic derivative, or tautomer thereof.

In some embodiments, the compound is of Formula (I-I-D-8):

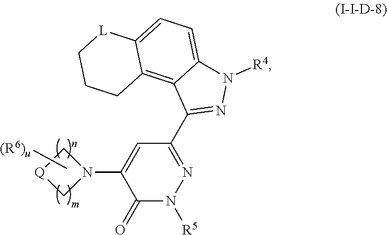

or a pharmaceutically acceptable salt, stereoisomer, solvate, prodrug, isotopic derivative, or tautomer thereof, wherein L is CH₂, O, S, or NH, and all other variables are as defined herein.

In some embodiments, the compound is of Formula (I-I-D-8-H):

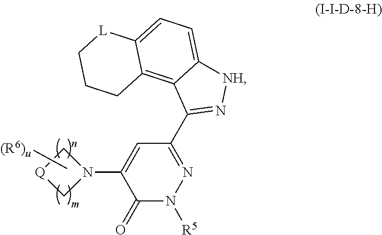

or a pharmaceutically acceptable salt, stereoisomer, solvate, prodrug, isotopic derivative, or tautomer thereof, wherein L is CH₂, O, S, or NH, and all other variables are as defined herein.

In some embodiments, the compound is of Formula (I-I-D-7-1):

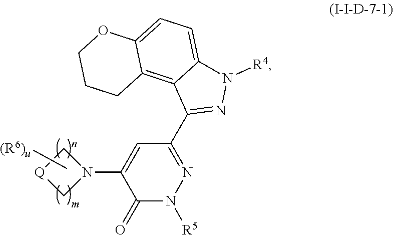

or a pharmaceutically acceptable salt, stereoisomer, solvate, prodrug, isotopic derivative, or tautomer thereof.

In some embodiments, the compound is of Formula (I-I-D-7-1-H):

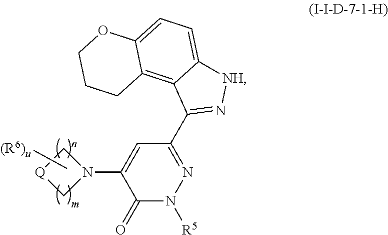

or a pharmaceutically acceptable salt, stereoisomer, solvate, prodrug, isotopic derivative, or tautomer thereof.

In some embodiments, the compound is of Formula (I-I-D-9):

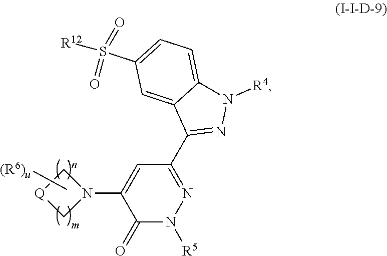

or a pharmaceutically acceptable salt, stereoisomer, solvate, prodrug, isotopic derivative, or tautomer thereof.

In some embodiments, the compound is of Formula (I-I-D-9-H):

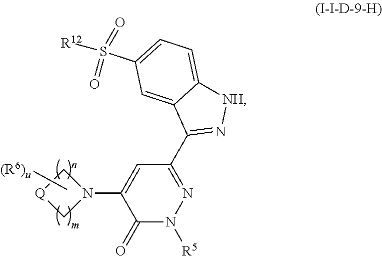

or a pharmaceutically acceptable salt, stereoisomer, solvate, prodrug, isotopic derivative, or tautomer thereof.

In some embodiments, the compound is of Formula (I-I-D-9-1):

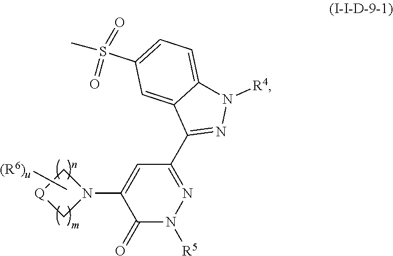

or a pharmaceutically acceptable salt, stereoisomer, solvate, prodrug, isotopic derivative, or tautomer thereof.

In some embodiments, the compound is of Formula (I-I-D-9-1-H):

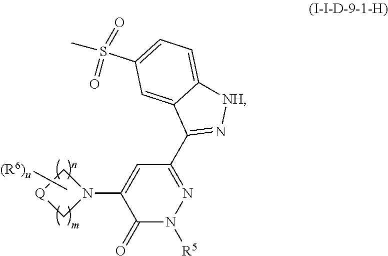

or a pharmaceutically acceptable salt, stereoisomer, solvate, prodrug, isotopic derivative, or tautomer thereof.

In some embodiments, the compound is of Formula (I-II-A-1):

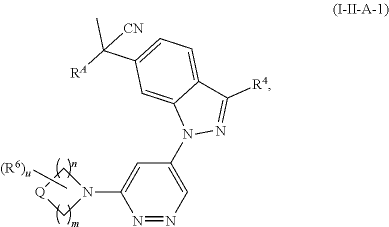

or a pharmaceutically acceptable salt, stereoisomer, solvate, prodrug, isotopic derivative, or tautomer thereof, wherein R^{A} is selected from H and C₁-C₃ alkyl and all other variables are as defined herein.

In some embodiments, the compound is of Formula (I-II-A-1-H):

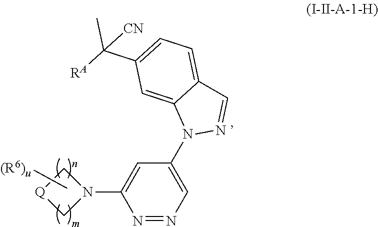

or a pharmaceutically acceptable salt, stereoisomer, solvate, prodrug, isotopic derivative, or tautomer thereof, wherein R^{A} is selected from H and C₁-C₃ alkyl and all other variables are as defined herein.

In some embodiments, the compound is of Formula (I-II-A-2):

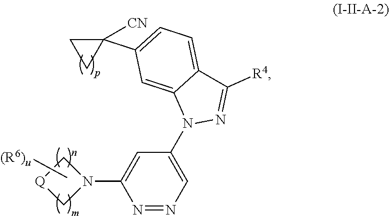

or a pharmaceutically acceptable salt, stereoisomer, solvate, prodrug, isotopic derivative, or tautomer thereof, wherein p is an integer selected from 1, 2, 3, 4, and 5 and all other variables are as defined herein.

In some embodiments, the compound is of Formula (I-II-A-2-H):

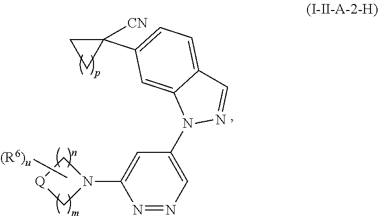

or a pharmaceutically acceptable salt, stereoisomer, solvate, prodrug, isotopic derivative, or tautomer thereof, wherein p is an integer selected from 1, 2, 3, 4, and 5, and all other variables are as defined herein.

In some embodiments, the compound is of Formula (I-II-A-2-H-1):

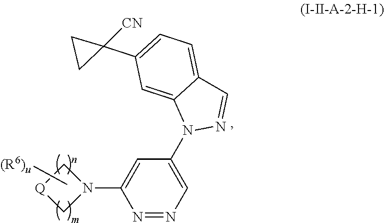

or a pharmaceutically acceptable salt, stereoisomer, solvate, prodrug, isotopic derivative, or tautomer thereof.

In some embodiments, the compound is of Formula (I-II-A-2-H-2):

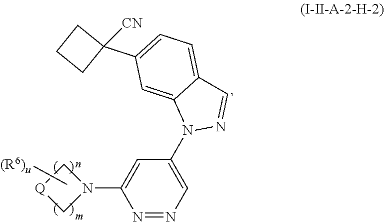

or a pharmaceutically acceptable salt, stereoisomer, solvate, prodrug, isotopic derivative, or tautomer thereof.

In some embodiments, the compound is of Formula (I-II-A-2-H-3):

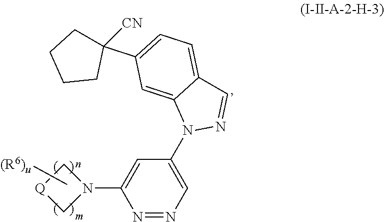

or a pharmaceutically acceptable salt, stereoisomer, solvate, prodrug, isotopic derivative, or tautomer thereof.

In some embodiments, the compound is of Formula (I-II-A-2-H-4):

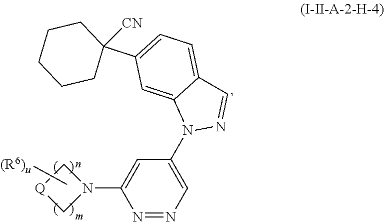

or a pharmaceutically acceptable salt, stereoisomer, solvate, prodrug, isotopic derivative, or tautomer thereof.

In some embodiments, the compound is of Formula (I-II-A-2-H-5):

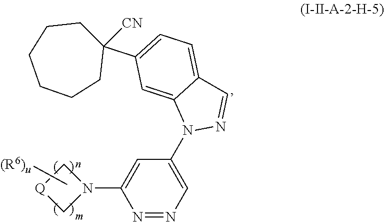

or a pharmaceutically acceptable salt, stereoisomer, solvate, prodrug, isotopic derivative, or tautomer thereof.

In some embodiments, the compound is of Formula (I-II-A-3):

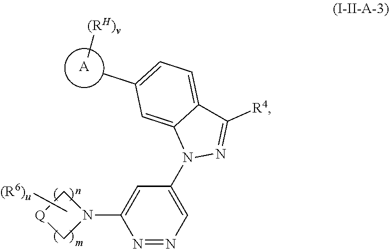

or a pharmaceutically acceptable salt, stereoisomer, solvate, prodrug, isotopic derivative, or tautomer thereof, wherein Ring A is heterocyclyl or heteroaryl; v is an integer selected from 0, 1, 2; each R^{H} is independently selected from halogen, OH, ═O, CN, C₁-C₆ alkyl; and all other variables are as defined herein.

In some embodiments, the compound is of Formula (I-II-A-3-H):

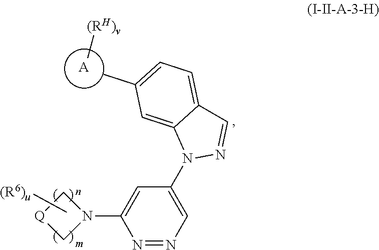

or a pharmaceutically acceptable salt, stereoisomer, solvate, prodrug, isotopic derivative, or tautomer thereof, wherein Ring A is heterocyclyl or heteroaryl; v is an integer selected from 0, 1, 2; each R^{H} is independently selected from halogen, OH, ═O, CN, C₁-C₆ alkyl; and all other variables are as defined herein.

In some embodiments, the compound is of Formula (I-II-A-3-1):

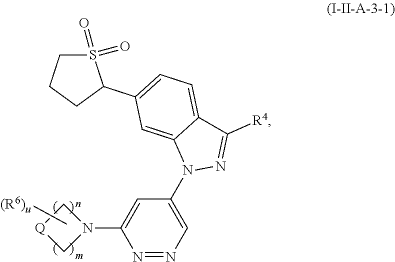

or a pharmaceutically acceptable salt, stereoisomer, solvate, prodrug, isotopic derivative, or tautomer thereof.

In some embodiments, the compound is of Formula (I-II-A-3-1-H):

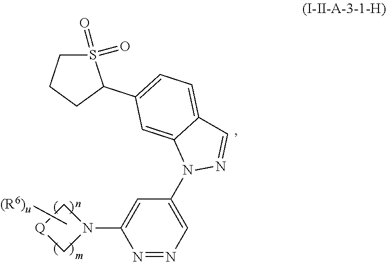

or a pharmaceutically acceptable salt, stereoisomer, solvate, prodrug, isotopic derivative, or tautomer thereof.

In some embodiments, the compound is of Formula (I-II-A-3-1-H′):

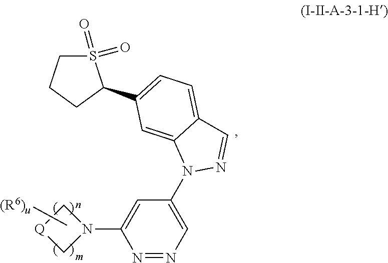

or a pharmaceutically acceptable salt, stereoisomer, solvate, prodrug, isotopic derivative, or tautomer thereof.

In some embodiments, the compound is of Formula (I-II-A-3-1-H″):

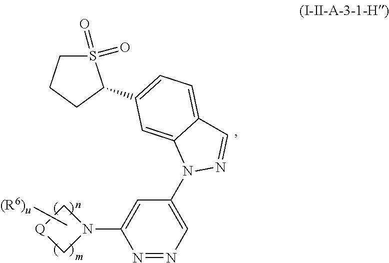

or a pharmaceutically acceptable salt, stereoisomer, solvate, prodrug, isotopic derivative, or tautomer thereof.

In some embodiments, the compound is of Formula (I-II-A-3-2):

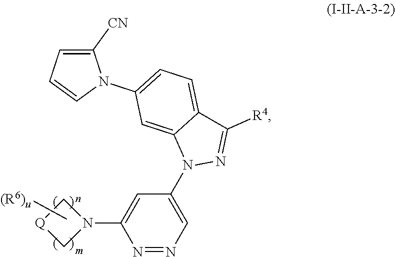

or a pharmaceutically acceptable salt, stereoisomer, solvate, prodrug, isotopic derivative, or tautomer thereof.

In some embodiments, the compound is of Formula (I-II-A-3-2-H):

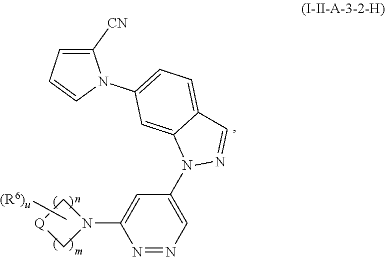

or a pharmaceutically acceptable salt, stereoisomer, solvate, prodrug, isotopic derivative, or tautomer thereof.

In some embodiments, the compound is of Formula (I-II-A-3-3):

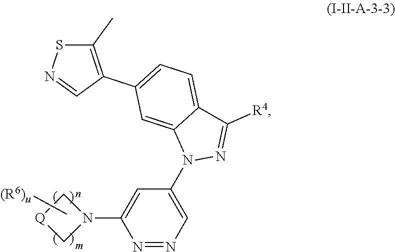

or a pharmaceutically acceptable salt, stereoisomer, solvate, prodrug, isotopic derivative, or tautomer thereof.

In some embodiments, the compound is of Formula (I-II-A-3-2-H):

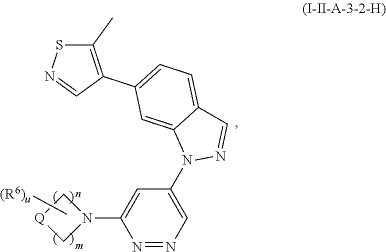

or a pharmaceutically acceptable salt, stereoisomer, solvate, prodrug, isotopic derivative, or tautomer thereof.

In some embodiments, the compound is of Formula (I-II-B-H):

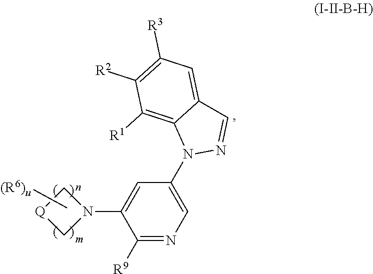

or a pharmaceutically acceptable salt, stereoisomer, solvate, prodrug, isotopic derivative, or tautomer thereof.

In some embodiments, the compound is of Formula (I-II-C-H):

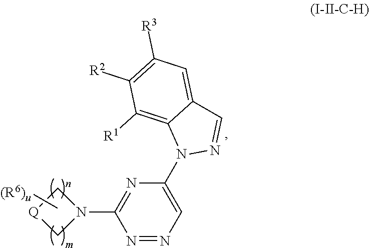

or a pharmaceutically acceptable salt, stereoisomer, solvate, prodrug, isotopic derivative, or tautomer thereof.

In some embodiments, the compound is of Formula (I-II-D-1):

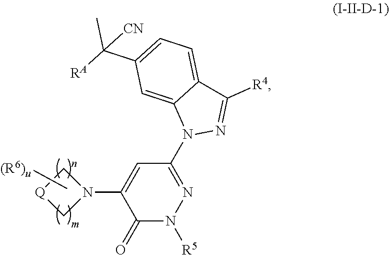

or a pharmaceutically acceptable salt, stereoisomer, solvate, prodrug, isotopic derivative, or tautomer thereof, wherein R^{A} is selected from H and C₁-C₃ alkyl and all other variables are as defined herein.

In some embodiments, the compound is of Formula (I-II-D-1-H):

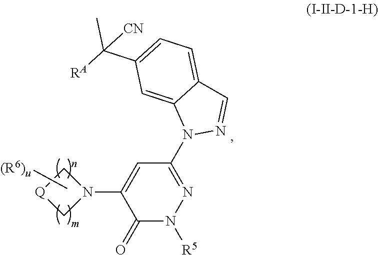

or a pharmaceutically acceptable salt, stereoisomer, solvate, prodrug, isotopic derivative, or tautomer thereof, wherein R^{A} is selected from H and C₁-C₃ alkyl and all other variables are as defined herein.

In some embodiments, the compound is of Formula (I-II-D-1-H-1):

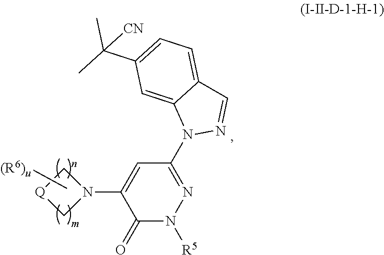

or a pharmaceutically acceptable salt, stereoisomer, solvate, prodrug, isotopic derivative, or tautomer thereof.

In some embodiments, the compound is of Formula (I-II-D-1-H-2):

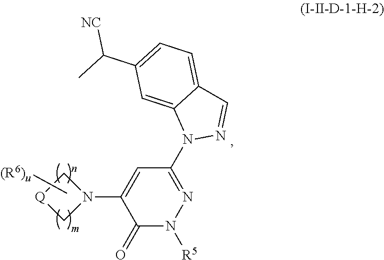

or a pharmaceutically acceptable salt, stereoisomer, solvate, prodrug, isotopic derivative, or tautomer thereof.

In some embodiments, the compound is of Formula (I-II-D-2):

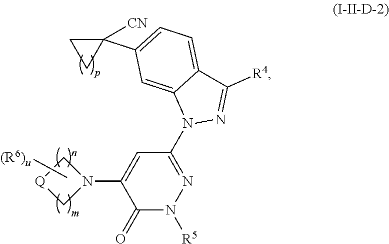

or a pharmaceutically acceptable salt, stereoisomer, solvate, prodrug, isotopic derivative, or tautomer thereof, wherein p is an integer selected from 1, 2, 3, 4, and 5, and all other variables are as defined herein.

In some embodiments, the compound is of Formula (I-II-D-2-H):

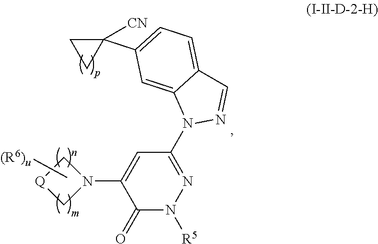

or a pharmaceutically acceptable salt, stereoisomer, solvate, prodrug, isotopic derivative, or tautomer thereof, wherein p is an integer selected from 1, 2, 3, 4, and 5, and all other variables are as defined herein.

In some embodiments, the compound is of Formula (I-II-D-2-H-1):

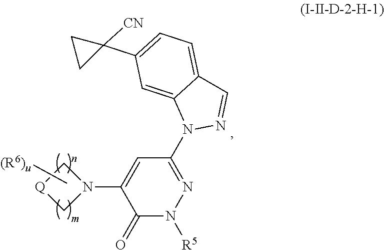

or a pharmaceutically acceptable salt, stereoisomer, solvate, prodrug, isotopic derivative, or tautomer thereof.

In some embodiments, the compound is of Formula (I-II-D-2-H-2):

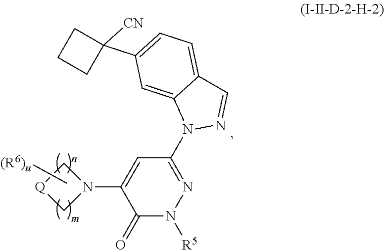

or a pharmaceutically acceptable salt, stereoisomer, solvate, prodrug, isotopic derivative, or tautomer thereof.

In some embodiments, the compound is of Formula (I-II-D-2-H-3):

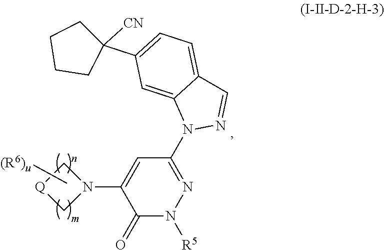

or a pharmaceutically acceptable salt, stereoisomer, solvate, prodrug, isotopic derivative, or tautomer thereof.

In some embodiments, the compound is of Formula (I-II-D-2-H-4):

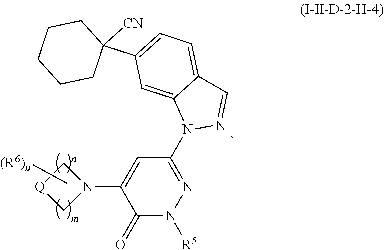

or a pharmaceutically acceptable salt, stereoisomer, solvate, prodrug, isotopic derivative, or tautomer thereof.

In some embodiments, the compound is of Formula (I-II-D-2-H-5):

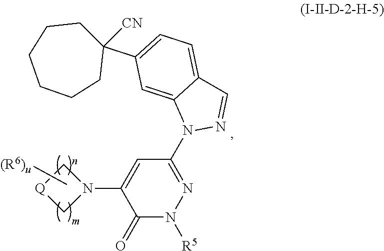

or a pharmaceutically acceptable salt, stereoisomer, solvate, prodrug, isotopic derivative, or tautomer thereof.

In some embodiments, the compound is selected from the compounds is of Formula (I-I-A-M), (I-I-B-M), (I-I-C-M), (I-I-D-M), (I-II-A-M), (I-II-B-M), (I-II-C-M), or (I-II-D-M):

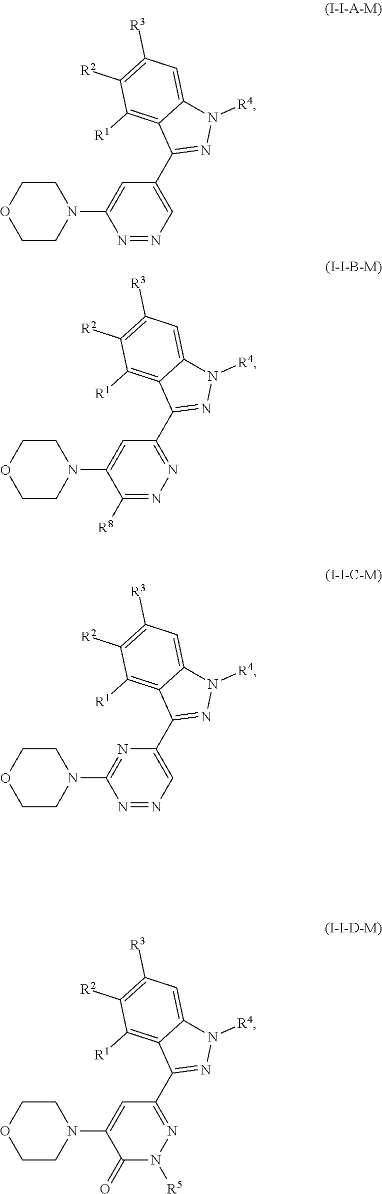

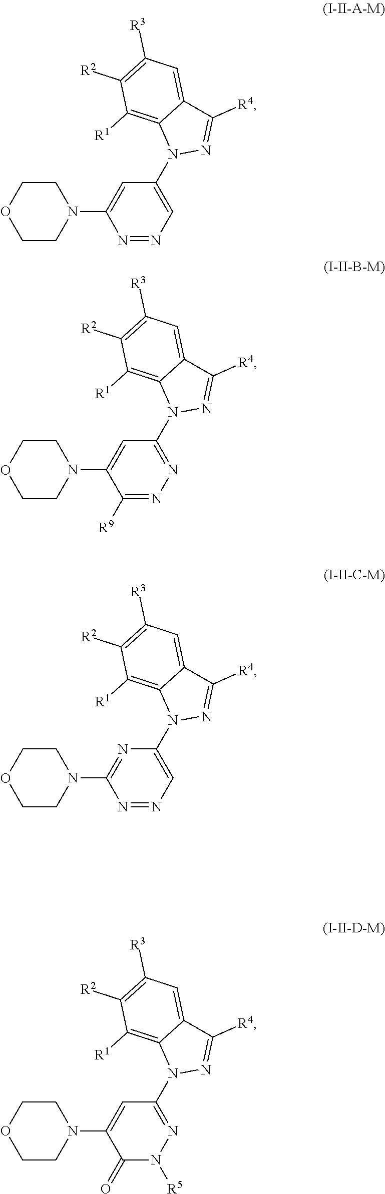

or a pharmaceutically acceptable salt, stereoisomer, solvate, or tautomer thereof.

In some embodiments, the compound is selected from the compounds of Formula (I-I-A-G), (I-I-B-G), (I-I-C-G), or (I-I-D-G):

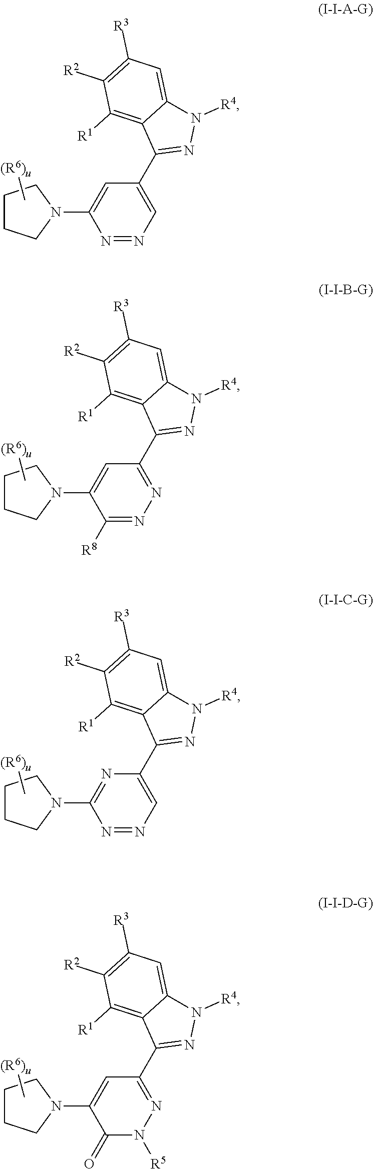

or a pharmaceutically acceptable salt, stereoisomer, solvate, or tautomer thereof.

In some embodiments, the compound is selected from the compounds of Formula (I-II-A-G), (I-II-B-G), (I-II-C-G), or (I-II-D-G):

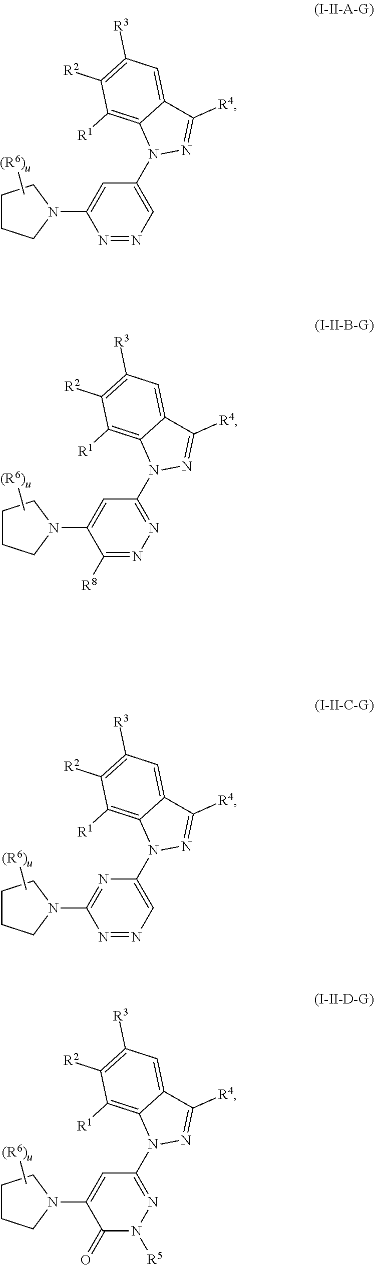

or a pharmaceutically acceptable salt, stereoisomer, solvate, or tautomer thereof.

In some embodiments, the compound selected from the compounds of Formula (I-I-A-A), (I-I-B-A), (I-I-C-A), (I-I-D-A), (I-II-A-A), (I-II-B-A), (I-II-C-A), or (I-II-D-A):

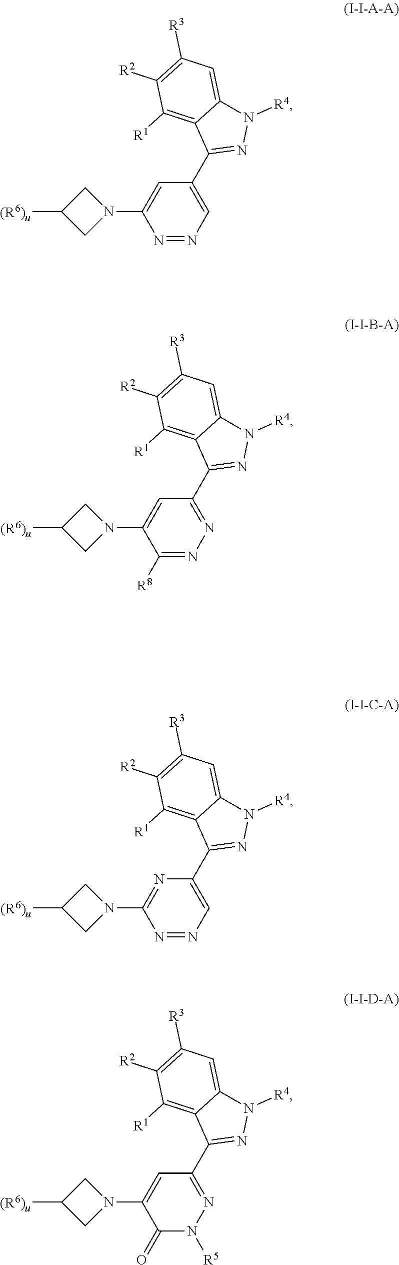

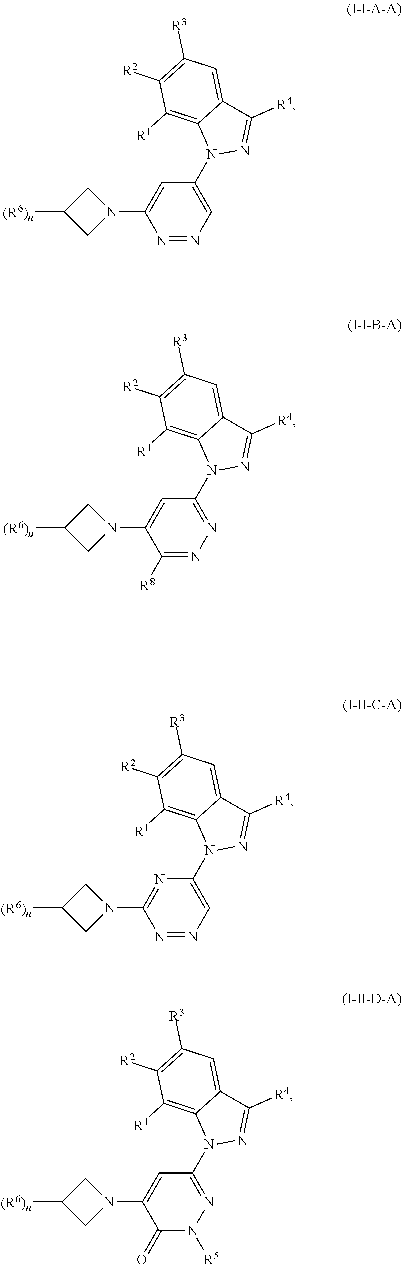

or a pharmaceutically acceptable salt, stereoisomer, solvate, or tautomer thereof, wherein u is 0 or 1 and all other variables are as defined herein.

In some preferred embodiments, the compound is compound 27:

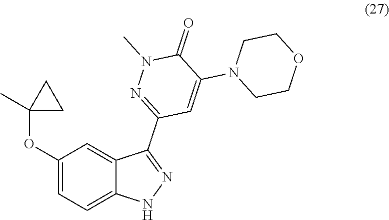 [SMILES: Cn1nc(-c2n[nH]c3ccc(OC4(C)CC4)cc23)cc(N2CCOCC2)c1=O]

or a pharmaceutically acceptable salt, stereoisomer, solvate, or tautomer thereof.

In some preferred embodiments, the compound is compound 50:

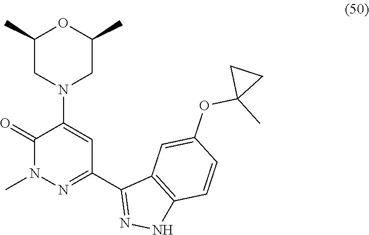 [SMILES: C[C@@H]1CN(c2cc(-c3n[nH]c4ccc(OC5(C)CC5)cc34)nn(C)c2=O)C[C@H](C)O1]

or a pharmaceutically acceptable salt, stereoisomer, solvate, or tautomer thereof.

In some preferred embodiments, the compound is compound 59:

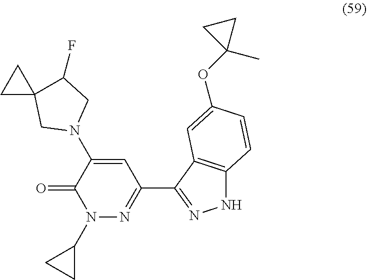 [SMILES: CC1(Oc2ccc3[nH]nc(-c4cc(N5CC(F)C6(CC6)C5)c(=O)n(C5CC5)n4)c3c2)CC1]

or a pharmaceutically acceptable salt, stereoisomer, solvate, or tautomer thereof.

In some preferred embodiments, the compound is compound 62:

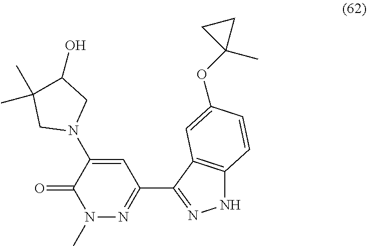

or a pharmaceutically acceptable salt, stereoisomer, solvate, or tautomer thereof.

In some preferred embodiments, the compound is compound 65:

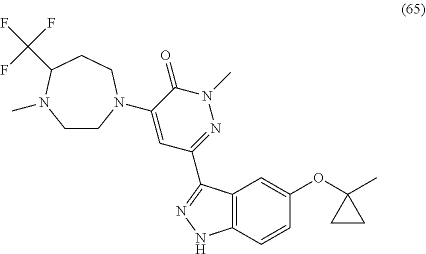 [SMILES: CN1CCN(c2cc(-c3n[nH]c4ccc(OC5(C)CC5)cc34)nn(C)c2=O)CCC1C(F)(F)F]

or a pharmaceutically acceptable salt, stereoisomer, solvate, or tautomer thereof.

In some preferred embodiments, the compound is compound 70:

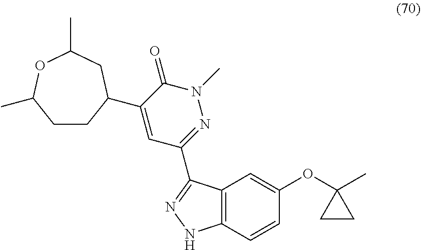

or a pharmaceutically acceptable salt, stereoisomer, solvate, or tautomer thereof.

In some preferred embodiments, the compound is compound 74:

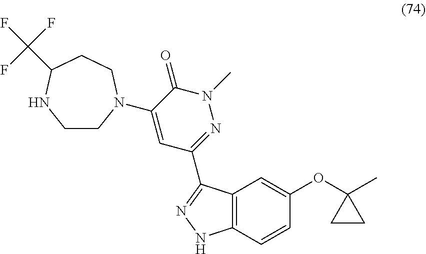 [SMILES: Cn1nc(-c2n[nH]c3ccc(OC4(C)CC4)cc23)cc(N2CCNC(C(F)(F)F)CC2)c1=O]

or a pharmaceutically acceptable salt, stereoisomer, solvate, or tautomer thereof.

In some preferred embodiments, the compound is compound 78:

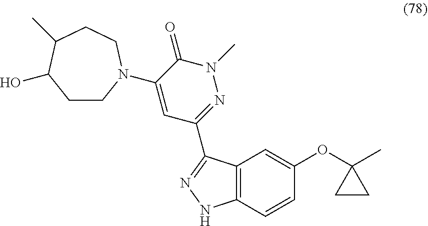 [SMILES: CC1CCN(c2cc(-c3n[nH]c4ccc(OC5(C)CC5)cc34)nn(C)c2=O)CCC1O]

or a pharmaceutically acceptable salt, stereoisomer, solvate, or tautomer thereof.

In some preferred embodiments, the compound is compound 79:

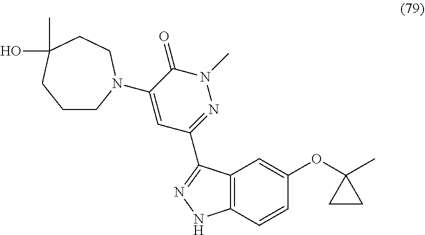 [SMILES: Cn1nc(-c2n[nH]c3ccc(OC4(C)CC4)cc23)cc(N2CCCC(C)(O)CC2)c1=O]

or a pharmaceutically acceptable salt, stereoisomer, solvate, or tautomer thereof.

In some preferred embodiments, the compound is compound 81:

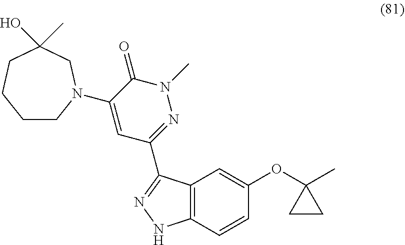 [SMILES: Cn1nc(-c2n[nH]c3ccc(OC4(C)CC4)cc23)cc(N2CCCCC(C)(O)C2)c1=O]

or a pharmaceutically acceptable salt, stereoisomer, solvate, or tautomer thereof.

In some preferred embodiments, the compound is compound 82:

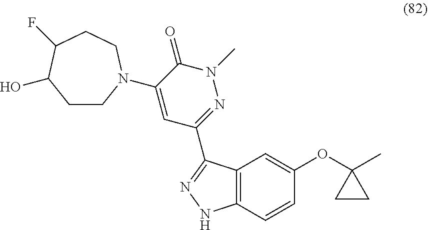 [SMILES: Cn1nc(-c2n[nH]c3ccc(OC4(C)CC4)cc23)cc(N2CCC(O)C(F)CC2)c1=O]

or a pharmaceutically acceptable salt, stereoisomer, solvate, or tautomer thereof.

In some preferred embodiments, the compound is compound 88:

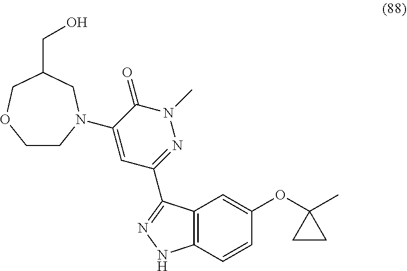 [SMILES: Cn1nc(-c2n[nH]c3ccc(OC4(C)CC4)cc23)cc(N2CCOCC(CO)C2)c1=O]

or a pharmaceutically acceptable salt, stereoisomer, solvate, or tautomer thereof.

In some preferred embodiments, the compound is compound 92:

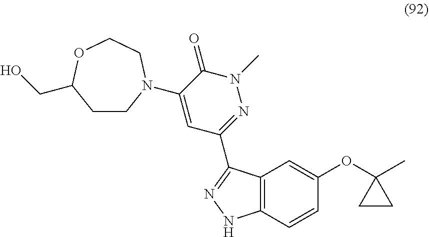 [SMILES: Cn1nc(-c2n[nH]c3ccc(OC4(C)CC4)cc23)cc(N2CCOC(CO)CC2)c1=O]

or a pharmaceutically acceptable salt, stereoisomer, solvate, or tautomer thereof.

In some preferred embodiments, the compound is compound 121:

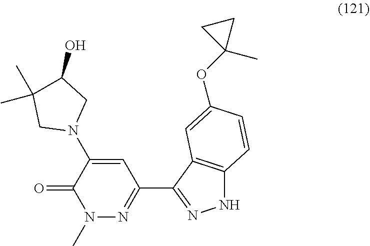

or a pharmaceutically acceptable salt, stereoisomer, solvate, or tautomer thereof.

In some preferred embodiments, the compound is compound 122:

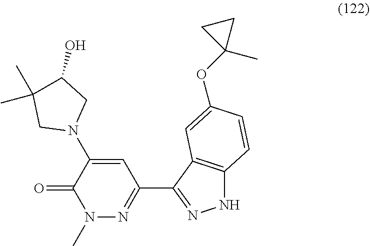

or a pharmaceutically acceptable salt, stereoisomer, solvate, or tautomer thereof.

In some preferred embodiments, the compound is compound 123:

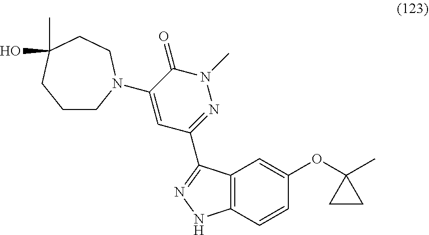 [SMILES: Cn1nc(-c2n[nH]c3ccc(OC4(C)CC4)cc23)cc(N2CCC[C@@](C)(O)CC2)c1=O]

or a pharmaceutically acceptable salt, stereoisomer, solvate, or tautomer thereof.

In some preferred embodiments, the compound is compound 124:

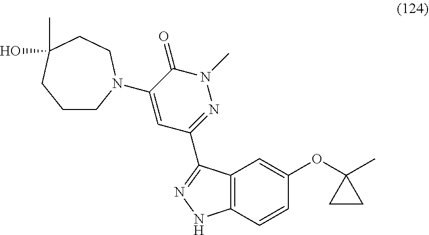 [SMILES: Cn1nc(-c2n[nH]c3ccc(OC4(C)CC4)cc23)cc(N2CCC[C@](C)(O)CC2)c1=O]

or a pharmaceutically acceptable salt, stereoisomer, solvate, or tautomer thereof.

In some preferred embodiments, the compound is compound 125:

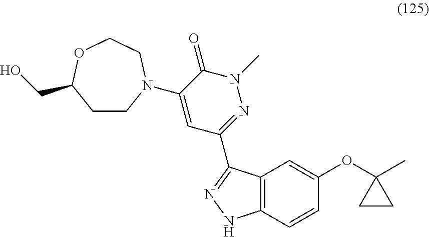 [SMILES: Cn1nc(-c2n[nH]c3ccc(OC4(C)CC4)cc23)cc(N2CCO[C@H](CO)CC2)c1=O]

or a pharmaceutically acceptable salt, stereoisomer, solvate, or tautomer thereof.

In some preferred embodiments, the compound is compound 126:

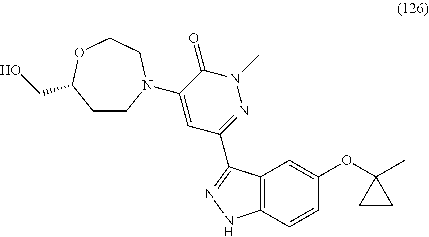 [SMILES: Cn1nc(-c2n[nH]c3ccc(OC4(C)CC4)cc23)cc(N2CCO[C@@H](CO)CC2)c1=O]

or a pharmaceutically acceptable salt, stereoisomer, solvate, or tautomer thereof.

In some preferred embodiments, the compound is compound 127:

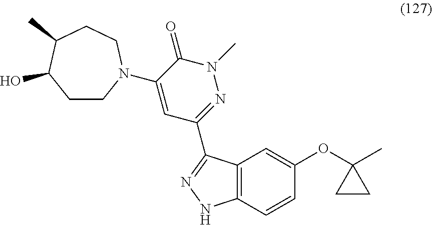 [SMILES: C[C@H]1CCN(c2cc(-c3n[nH]c4ccc(OC5(C)CC5)cc34)nn(C)c2=O)CC[C@H]1O]

or a pharmaceutically acceptable salt, stereoisomer, solvate, or tautomer thereof.

In some preferred embodiments, the compound is compound 128:

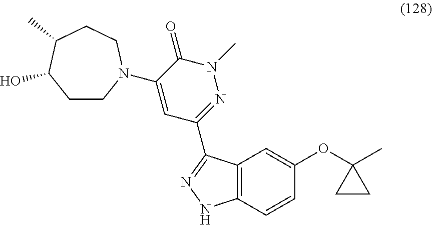 [SMILES: C[C@@H]1CCN(c2cc(-c3n[nH]c4ccc(OC5(C)CC5)cc34)nn(C)c2=O)CC[C@@H]1O]

or a pharmaceutically acceptable salt, stereoisomer, solvate, or tautomer thereof.

In some preferred embodiments, the compound is compound 129:

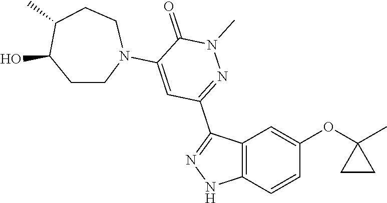 [SMILES: C[C@@H]1CCN(c2cc(-c3n[nH]c4ccc(OC5(C)CC5)cc34)nn(C)c2=O)CC[C@H]1O]

or a pharmaceutically acceptable salt, stereoisomer, solvate, or tautomer thereof.

In some preferred embodiments, the compound is compound 130:

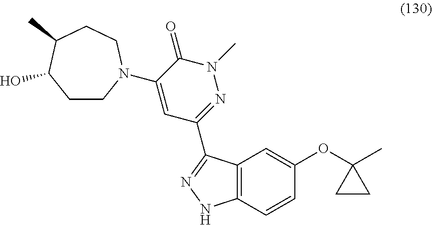 [SMILES: C[C@H]1CCN(c2cc(-c3n[nH]c4ccc(OC5(C)CC5)cc34)nn(C)c2=O)CC[C@@H]1O]

or a pharmaceutically acceptable salt, stereoisomer, solvate, or tautomer thereof.

In some preferred embodiments, the compound is compound 131:

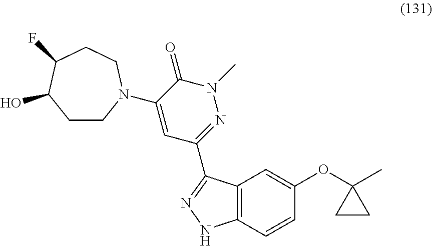 [SMILES: Cn1nc(-c2n[nH]c3ccc(OC4(C)CC4)cc23)cc(N2CC[C@@H](O)[C@@H](F)CC2)c1=O]

or a pharmaceutically acceptable salt, stereoisomer, solvate, or tautomer thereof.

In some preferred embodiments, the compound is compound 132:

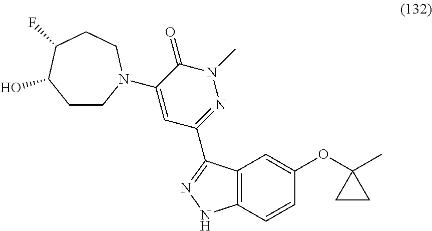 [SMILES: Cn1nc(-c2n[nH]c3ccc(OC4(C)CC4)cc23)cc(N2CC[C@@H](F)[C@@H](O)CC2)c1=O]

or a pharmaceutically acceptable salt, stereoisomer, solvate, or tautomer thereof.

In some preferred embodiments, the compound is compound 133:

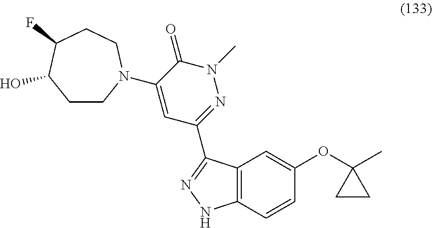 [SMILES: Cn1nc(-c2n[nH]c3ccc(OC4(C)CC4)cc23)cc(N2CC[C@H](O)[C@@H](F)CC2)c1=O]

or a pharmaceutically acceptable salt, stereoisomer, solvate, or tautomer thereof.

In some preferred embodiments, the compound is compound 134:

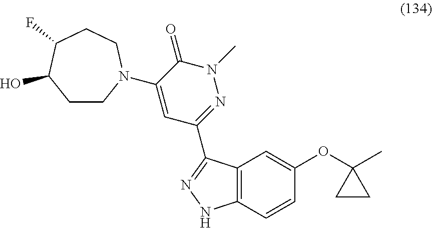 [SMILES: Cn1nc(-c2n[nH]c3ccc(OC4(C)CC4)cc23)cc(N2CC[C@@H](O)[C@H](F)CC2)c1=O]

or a pharmaceutically acceptable salt, stereoisomer, solvate, or tautomer thereof.

In some embodiments, the compound is selected from the compounds described in Table 1 and pharmaceutically acceptable salts, stereoisomers, solvates, prodrugs, isotopic derivatives, or tautomers thereof.

In some embodiments, the compound is selected from the compounds described in Table 1 and prodrugs and pharmaceutically acceptable salts thereof.

In some embodiments, the compound is selected from the compounds described in Table 1 and pharmaceutically acceptable salts thereof.

In some embodiments, the compound is selected from the prodrugs of the compounds described in Table 1 and pharmaceutically acceptable salts thereof.

In some embodiments, the compound is selected from the compounds described in Table 1.

TABLE 1
Non-limiting illustrative compounds of the present disclosure

| # | IUPAC Name |
|---|---|
| 1 | 1-[5-(5-isopropoxy-1H-indazol-3-yl)pyridazin-3-yl]azetidin-3-ol |
| 2 | (R)-1-(5-(5-isopropoxy-1H-indazol-3-yl)pyridazin-3-yl)pyrrolidin-3-ol |
| 3 | 5-isopropoxy-3-[6-(3-methylpyrrolidin-1-yl)pyridazin-4-yl]-1H-indazole |
| 4 | 4-[5-(5-isopropoxy-1H-indazol-3-yl)pyridazin-3-yl]morpholine |
| 5 | 5-isopropoxy-3-(6-pyrrolidin-1-ylpyridazin-4-yl)-1H-indazole |
| 6 | 1,1-difluoro-N-[3-(6-pyrrolidin-1-ylpyridazin-4-yl)-1H-indazol-5-yl]methanesulfonamide |
| 7 | 5,5-dimethyl-1-[3-(6-pyrrolidin-1-ylpyridazin-4-yl)-1H-indazol-5-yl]pyrrolidin-2-one |
| 8 | 5-(1,1-difluoro-2-methyl-propyl)-3-(6-pyrrolidin-1-ylpyridazin-4-yl)-1H-indazole |
| 9 | 5-(1-fluorocyclobutyl)-3-(6-pyrrolidin-1-ylpyridazin-4-yl)-1H-indazole |
| 10 | 4-[6-(5-isopropoxy-1H-indazol-3-yl)-3-methyl-pyridazin-4-yl]morpholine |
| 11 | 4-[6-(5-isopropoxy-1H-indazol-3-yl)pyridazin-4-yl]morpholine |
| 12 | 5-isopropoxy-3-(5-pyrrolidin-1-ylpyridazin-3-yl)-1H-indazole |
| 13 | 4-[5-(5-isopropoxy-1H-indazol-3-yl)-1,2,4-triazin-3-yl]morpholine |
| 14 | 6-(5-isopropoxy-1H-indazol-3-yl)-2-methyl-4-morpholino-pyridazin-3-one |
| 15 | 6-(4-isopropoxy-1H-indazol-3-yl)-2-methyl-4-morpholino-pyridazin-3-one |
| 16 | 2-cyclopentyl-6-(5-isopropoxy-1H-indazol-3-yl)-4-morpholino-pyridazin-3-one |
| 17 | 6-(5-isopropoxy-1H-indazol-3-yl)-4-morpholino-2-(trifluoromethyl)pyridazin-3-one |
| 18 | 2-isobutyl-6-(5-isopropoxy-1H-indazol-3-yl)-4-morpholino-pyridazin-3-one |
| 19 | 2-cyclopropyl-6-(5-isopropoxy-1H-indazol-3-yl)-4-morpholino-pyridazin-3-one |
| 20 | 2-(difluoromethyl)-6-(5-isopropoxy-1H-indazol-3-yl)-4-morpholino-pyridazin-3-one |
| 21 | 6-(5-isopropoxy-1H-indazol-3-yl)-2-isopropyl-4-morpholino-pyridazin-3-one |
| 22 | 2-methyl-4-morpholino-6-[5-(trifluoromethoxy)-1H-indazol-3-yl]pyridazin-3-one |
| 23 | 2-ethyl-6-(5-isopropoxy-1H-indazol-3-yl)-4-morpholino-pyridazin-3-one |
| 24 | 6-(5-isopropoxy-4-methyl-1H-indazol-3-yl)-2-methyl-4-morpholino-pyridazin-3-one |
| 25 | 6-(5-tert-butoxy-1H-indazol-3-yl)-2-methyl-4-morpholino-pyridazin-3-one |
| 26 | 6-(5-isopropoxy-6-methyl-1H-indazol-3-yl)-2-methyl-4-morpholino-pyridazin-3-one |
| 27 | 2-methyl-6-[5-(1-methylcyclopropoxy)-1H-indazol-3-yl]-4-morpholino-pyridazin-3-one |
| 28 | 6-[5-(cyclobutoxy)-1H-indazol-3-yl]-2-methyl-4-morpholino-pyridazin-3-one |
| 29 | 2-methyl-4-morpholino-6-(3,7,8,9-tetrahydropyrano[3,2-e]indazol-1-yl)pyridazin-3-one |
| 30 | 6-[5-(cyclopropoxy)-1H-indazol-3-yl]-2-methyl-4-morpholino-pyridazin-3-one |
| 31 | 2-methyl-4-morpholino-6-(1,6,7,8-tetrahydropyrano[2,3-f]indazol-3-yl)pyridazin-3-one |
| 32 | 6-[5-(difluoromethoxy)-1H-indazol-3-yl]-2-methyl-4-morpholino-pyridazin-3-one |
| 33 | 2-methyl-6-(5-methylsulfonyl-1H-indazol-3-yl)-4-morpholino-pyridazin-3-one |
| 34 | 2-[1-(6-pyrrolidin-1-ylpyridazin-4-yl)indazol-6-yl]thiolane 1,1-dioxide |
| 35 | 2-methyl-2-[1-(6-pyrrolidin-1-ylpyridazin-4-yl)indazol-6-yl]propanenitrile |
| 36 | 1-[1-(6-pyrrolidin-1-ylpyridazin-4-yl)indazol-6-yl]cyclopropanecarbonitrile |
| 37 | 1-[1-(6-pyrrolidin-1-ylpyridazin-4-yl)indazol-6-yl]cyclohexanecarbonitrile |
| 38 | 5-methyl-4-[1-(6-pyrrolidin-1-ylpyridazin-4-yl)indazol-6-yl]isothiazole |
| 39 | 1-[1-(6-pyrrolidin-1-ylpyridazin-4-yl)indazol-6-yl]cyclopentanecarbonitrile |
| 40 | 1-[1-(6-pyrrolidin-1-ylpyridazin-4-yl)indazol-6-yl]pyrrole-2-carbonitrile |
| 41 | 1-[1-(1-methyl-5-morpholino-6-oxo-pyridazin-3-yl)indazol-6-yl]cyclopropanecarbonitrile |
| 42 | 2-[1-(1-methyl-5-morpholino-6-oxo-pyridazin-3-yl)indazol-6-yl]propanenitrile |
| 43 | 2-methyl-2-[1-(1-methyl-5-morpholino-6-oxo-pyridazin-3-yl)indazol-6-yl]propanenitrile |
| 44 | 6-(5-isopropoxy-1H-indazol-3-yl)-2-methyl-4-pyrrolidin-1-yl-pyridazin-3-one |
| 45 | 1-[3-(1-methyl-5-morpholino-6-oxo-pyridazin-3-yl)-1H-indazol-5-yl]pyrrole-2- |
| | carbonitrile |
| 46 | 4-(2,3,3a,5,6,6a-hexahydrofuro[3,2-b]pyrrol-4-yl)-2-methyl-6-[5-(1- |
| | methylcyclopropoxy)-1H-indazol-3-yl]pyridazin-3-one |
| 47 | 4-(2,3,3a,5,6,6a-hexahydrofuro[3,2-b]pyrrol-4-yl)-2-cyclopropyl-6-[5-(1- |
| | methylcyclopropoxy)-1H-indazol-3-yl]pyridazin-3-one |
| 48 | 4-(3,3-dimethyl-3a,4,6,6a-tetrahydro-2H-furo[3,4-b]pyrrol-1-yl)-2-methyl-6-[5-(1- |
| | methylcyclopropoxy)-1H-indazol-3-yl]pyridazin-3-one |
| 49 | 2-cyclopropyl-4-(3,3-dimethyl-3a,4,6,6a-tetrahydro-2H-furo[3,4-b]pyrrol-1-yl)-6- |
| | [5-(1-methylcyclopropoxy)-1H-indazol-3-yl]pyridazin-3-one |
| 50 | 4-[(2S,6R)-2,6-dimethylmorpholin-4-yl]-2-methyl-6-[5-(1-methylcyclopropoxy)- |
| | 1H-indazol-3-yl]pyridazin-3-one |
| 51 | 2-cyclopropyl-4-[(2S,6R)-2,6-dimethylmorpholin-4-yl]-6-[5-(1- |
| | methylcyclopropoxy)-1H-indazol-3-yl]pyridazin-3-one |
| 52 | 2-methyl-6-[5-(1-methylcyclopropoxy)-1H-indazol-3-yl]-4-(5-oxa-8- |
| | azaspiro[2.6]nonan-8-yl)pyridazin-3-one |
| 53 | 2-cyclopropyl-6-[5-(1-methylcyclopropoxy)-1H-indazol-3-yl]-4-(5-oxa-8- |
| | azaspiro[2.6]nonan-8-yl)pyridazin-3-one |
| 54 | 4-(3,3-difluoro-1-piperidyl)-2-methyl-6-[5-(1-methylcyclopropoxy)-1H-indazol-3- |
| | yl]pyridazin-3-one |
| 55 | 4-(4-fluoro-3,3-dimethyl-pyrrolidin-1-yl)-2-methyl-6-[5-(1-methylcyclopropoxy)- |
| | 1H-indazol-3-yl]pyridazin-3-one |
| 56 | 4-(6-azaspiro[3.4]octan-6-yl)-2-methyl-6-[5-(1-methylcyclopropoxy)-1H-indazol- |
| | 3-yl]pyridazin-3-one |
| 57 | 4-(7-fluoro-5-azaspiro[2.4]heptan-5-yl)-2-methyl-6-[5-(1-methylcyclopropoxy)- |
| | 1H-indazol-3-yl]pyridazin-3-one |
| 58 | 4-(6-azaspiro[3.4]octan-6-yl)-2-cyclopropyl-6-[5-(1-methylcyclopropoxy)-1H- |
| | indazol-3-yl]pyridazin-3-one |
| 59 | 2-cyclopropyl-4-(7-fluoro-5-azaspiro[2.4]heptan-5-yl)-6-[5-(1- |
| | methylcyclopropoxy)-1H-indazol-3-yl]pyridazin-3-one |
| 60 | 2-cyclopropyl-4-(3,3-difluoro-1-piperidyl)-6-[5-(1-methylcyclopropoxy)-1H- |
| | indazol-3-yl]pyridazin-3-one |
| 61 | 2-cyclopropyl-4-(4-fluoro-3,3-dimethyl-pyrrolidin-1-yl)-6-[5-(1- |
| | methylcyclopropoxy)-1H-indazol-3-yl]pyridazin-3-one |
| 62 | 4-(4-hydroxy-3,3-dimethyl-pyrrolidin-1-yl)-2-methyl-6-[5-(1- |
| | methylcyclopropoxy)-1H-indazol-3-yl]pyridazin-3-one |
| 63 | 2-methyl-6-[5-(1-methylcyclopropoxy)-1H-indazol-3-yl]-4-(4-methyl-1,4- |
| | diazepan-1-yl)pyridazin-3-one |
| 64 | 4-(6,6-difluoro-4-methyl-1,4-diazepan-1-yl)-2-methyl-6-[5-(1- |
| | methylcyclopropoxy)-1H-indazol-3-yl]pyridazin-3-one |
| 65 | 2-methyl-6-[5-(1-methylcyclopropoxy)-1H-indazol-3-yl]-4-[4-methyl-5- |
| | (trifluoromethyl)-1,4-diazepan-1-yl]pyridazin-3-one |
| 66 | 2-methyl-6-[5-(1-methylcyclopropoxy)-1H-indazol-3-yl]-4-[4-(2,2,2- |
| | trifluoroethyl)-1,4-diazepan-1-yl]pyridazin-3-one |
| 67 | 4-(3,3-dimethyl-1,4-diazepan-1-yl)-2-methyl-6-[5-(1-methylcyclopropoxy)-1H- |
| | indazol-3-yl]pyridazin-3-one |
| 68 | 2-methyl-6-[5-(1-methylcyclopropoxy)-1H-indazol-3-yl]-4-(3,3,5,5-tetramethyl- |
| | 1,4-diazepan-1-yl)pyridazin-3-one |
| 69 | 4-(6,6-dimethyl-1,4-diazepan-1-yl)-2-methyl-6-[5-(1-methylcyclopropoxy)-1H- |
| | indazol-3-yl]pyridazin-3-one |
| 70 | 4-(2,7-dimethyl-1,4-oxazepan-4-yl)-2-methyl-6-[5-(1-methylcyclopropoxy)-1H- |
| | indazol-3-yl]pyridazin-3-one |
| 71 | 4-(6,6-dimethyl-1,4-oxazepan-4-yl)-2-methyl-6-[5-(1-methylcyclopropoxy)-1H- |
| | indazol-3-yl]pyridazin-3-one |
| 72 | 4-(6,6-difluoro-1,4-diazepan-1-yl)-2-methyl-6-[5-(1-methylcyclopropoxy)-1H- |
| | indazol-3-yl]pyridazin-3-one |
| 73 | 4-(2,2-dimethyl-1,4-oxazepan-4-yl)-2-methyl-6-[5-(1-methylcyclopropoxy)-1H- |
| | indazol-3-yl]pyridazin-3-one |
| 74 | 2-methyl-6-[5-(1-methylcyclopropoxy)-1H-indazol-3-yl]-4-[5-(trifluoromethyl)- |
| | 1,4-diazepan-1-yl]pyridazin-3-one |
| 75 | 4-[3-(hydroxymethyl)azepan-1-yl]-2-methyl-6-[5-(1-methylcyclopropoxy)-1H- |
| | indazol-3-yl]pyridazin-3-one |
| 76 | 2-methyl-6-[5-(1-methylcyclopropoxy)-1H-indazol-3-yl]-4-(1,4-oxazepan-4- |
| | yl)pyridazin-3-one |
| 77 | 4-(4-hydroxyazepan-1-yl)-2-methyl-6-[5-(1-methylcyclopropoxy)-1H-indazol-3- |
| | yl]pyridazin-3-one |
| 78 | 4-(4-hydroxy-5-methyl-azepan-1-yl)-2-methyl-6-[5-(1-methylcyclopropoxy)-1H- |
| | indazol-3-yl]pyridazin-3-one |
| 79 | 4-(4-hydroxy-4-methyl-azepan-1-yl)-2-methyl-6-[5-(1-methylcyclopropoxy)-1H- |
| | indazol-3-yl]pyridazin-3-one |
| 80 | 4-[6-(hydroxymethyl)-4-methyl-1,4-diazepan-1-yl]-2-methyl-6-[5-(1- |
| | methylcyclopropoxy)-1H-indazol-3-yl]pyridazin-3-one |
| 81 | 4-(3-hydroxy-3-methyl-azepan-1-yl)-2-methyl-6-[5-(1-methylcyclopropoxy)-1H- |
| | indazol-3-yl]pyridazin-3-one |
| 82 | 4-(4-fluoro-5-hydroxy-azepan-1-yl)-2-methyl-6-[5-(1-methylcyclopropoxy)-1H- |
| | indazol-3-yl]pyridazin-3-one |
| 83 | 4-(6-hydroxy-4-methyl-1,4-diazepan-1-yl)-2-methyl-6-[5-(1-methylcyclopropoxy)- |
| | 1H-indazol-3-yl]pyridazin-3-one |
| 84 | 4-[4-(2-hydroxy-2-methyl-propyl)-1,4-diazepan-1-yl]-2-methyl-6-[5-(1- |
| | methylcyclopropoxy)-1H-indazol-3-yl]pyridazin-3-one |
| 85 | 4-[3-(1-hydroxy-1-methyl-ethyl)pyrrolidin-1-yl]-2-methyl-6-[5-(1- |
| | methylcyclopropoxy)-1H-indazol-3-yl]pyridazin-3-one |
| 86 | 4-(5-hydroxy-4,4-dimethyl-azepan-1-yl)-2-methyl-6-[5-(1-methylcyclopropoxy)- |
| | 1H-indazol-3-yl]pyridazin-3-one |
| 87 | 4-[3-(hydroxymethyl)-4-methyl-piperazin-1-yl]-2-methyl-6-[5-(1- |
| | methylcyclopropoxy)-1H-indazol-3-yl]pyridazin-3-one |
| 88 | 4-[6-(hydroxymethyl)-1,4-oxazepan-4-yl]-2-methyl-6-[5-(1-methylcyclopropoxy)- |
| | 1H-indazol-3-yl]pyridazin-3-one |
| 89 | 4-[2-(hydroxymethyl)-1,4-oxazepan-4-yl]-2-methyl-6-[5-(1-methylcyclopropoxy)- |
| | 1H-indazol-3-yl]pyridazin-3-one |
| 90 | 4-(6-hydroxy-6-methyl-1,4-oxazepan-4-yl)-2-methyl-6-[5-(1- |
| | methylcyclopropoxy)-1H-indazol-3-yl]pyridazin-3-one |
| 91 | 4-(6-hydroxy-1,4-oxazepan-4-yl)-2-methyl-6-[5-(1-methylcyclopropoxy)-1H- |
| | indazol-3-yl]pyridazin-3-one |
| 92 | 4-[7-(hydroxymethyl)-1,4-oxazepan-4-yl]-2-methyl-6-[5-(1-methylcyclopropoxy)- |
| | 1H-indazol-3-yl]pyridazin-3-one |
| 93 | 4-(6-hydroxy-6-methyl-1,4-diazepan-1-yl)-2-methyl-6-[5-(1-methylcyclopropoxy)- |
| | 1H-indazol-3-yl]pyridazin-3-one |
| 94 | 4-[4-hydroxy-4-(hydroxymethyl)azepan-1-yl]-2-methyl-6-[5-(1- |
| | methylcyclopropoxy)-1H-indazol-3-yl]pyridazin-3-one |
| 95 | 4-(1,1-dioxo-1,4-thiazepan-4-yl)-2-methyl-6-[5-(1-methylcyclopropoxy)-1H- |
| | indazol-3-yl]pyridazin-3-one |
| 96 | 4-(4-isopropyl-1,4-diazepan-1-yl)-2-methyl-6-[5-(1-methylcyclopropoxy)-1H- |
| | indazol-3-yl]pyridazin-3-one |
| 97 | 4-(1, 1-dioxo-1lambda6-thia-7-azaspiro[3.4]octan-7-yl)-2-methyl-6-[5-(1- |
| | methylcyclopropoxy)-1H-indazol-3-yl]pyridazin-3-one |
| 98 | 4-(2,2-dioxo-2lambda6-thia-7-azaspiro[4.4]nonan-7-yl)-2-methyl-6-[5-(1- |
| | methylcyclopropoxy)-1H-indazol-3-yl]pyridazin-3-one |
| 99 | 4-(2,2-dioxo-2lambda6-thia-7-azaspiro[3.4]octan-7-yl)-2-methyl-6-[5-(1- |
| | methylcyclopropoxy)-1H-indazol-3-yl]pyridazin-3-one |
| 100 | 2-methyl-6-[5-(1-methylcyclopropoxy)-1H-indazol-3-yl]-4-(2-methylsulfonyl-6- |
| | azaspiro[3.4]octan-6-yl)pyridazin-3-one |
| 101 | 4-[5-(hydroxymethyl)-2-oxa-7-azaspiro[3.4]octan-7-yl]-2-methyl-6-[5-(1- |
| | methylcyclopropoxy)-1H-indazol-3-yl]pyridazin-3-one |
| 102 | 4-(2-hydroxy-6-azaspiro[3.4]octan-6-yl)-2-methyl-6-[5-(1-methylcyclopropoxy)- |
| | 1H-indazol-3-yl]pyridazin-3-one |
| 103 | 4-(8-hydroxy-2-azaspiro[4.4]nonan-2-yl)-2-methyl-6-[5-(1-methylcyclopropoxy)- |
| | 1H-indazol-3-yl]pyridazin-3-one |
| 104 | 4-(3-hydroxy-6-azaspiro[3.4]octan-6-yl)-2-methyl-6-[5-(1-methylcyclopropoxy)- |
| | 1H-indazol-3-yl]pyridazin-3-one |
| 105 | 4-[2-(hydroxymethyl)-6-azaspiro[3.4]octan-6-yl]-2-methyl-6-[5-(1- |
| | methylcyclopropoxy)-1H-indazol-3-yl]pyridazin-3-one |
| 106 | 2-methyl-6-[5-(1-methylcyclopropoxy)-1H-indazol-3-yl]-4-(1-oxa-7- |
| | azaspiro[3.4]octan-7-yl)pyridazin-3-one |
| 107 | 4-[8-(hydroxymethyl)-6-azaspiro[3.4]octan-6-yl]-2-methyl-6-[5-(1- |
| | methylcyclopropoxy)-1H-indazol-3-yl]pyridazin-3-one |
| 108 | 2-methyl-6-[5-(1-methylcyclopropoxy)-1H-indazol-3-yl]-4-(2-oxa-7- |
| | azaspiro[4.4]nonan-7-yl)pyridazin-3-one |
| 109 | 2-methyl-6-[5-(1-methylcyclopropoxy)-1H-indazol-3-yl]-4-(2-oxa-7- |
| | azaspiro[3.4]octan-7-yl)pyridazin-3-one |
| 110 | 2-methyl-6-[5-(1-methylcyclopropoxy)-1H-indazol-3-yl]-4-(1-oxa-7- |
| | azaspiro[4.4 ]nonan-7-yl)pyridazin-3-one |
| 111 | 2-methyl-6-[5-(1-methylcyclopropoxy)-1H-indazol-3-yl]-4-(2-methyl-2,6- |
| | diazaspiro[3.4]octan-6-yl)pyridazin-3-one |
| 112 | 4-(3,3-dimethyl-2-oxa-7-azaspiro[4.4]nonan-7-yl)-2-methyl-6-[5-(1- |
| | methylcyclopropoxy)-1H-indazol-3-yl]pyridazin-3-one |
| 113 | 2-methyl-6-[5-(1-methylcyclopropoxy)-1H-indazol-3-yl]-4-(7-methyl-2,7- |
| | diazaspiro[4.4]nonan-2-yl)pyridazin-3-one |
| 114 | 4-[8-(ethoxymethyl)-6-azaspiro[3.4]octan-6-yl]-2-methyl-6-[5-(1- |
| | methylcyclopropoxy)-1H-indazol-3-yl]pyridazin-3-one |
| 115 | 4-[8-(methoxymethyl)-6-azaspiro[3.4]octan-6-yl]-2-methyl-6-[5-(1- |
| | methylcyclopropoxy)-1H-indazol-3-yl]pyridazin-3-one |
| 116 | 2-methyl-6-[5-(1-methylcyclopropoxy)-1H-indazol-3-yl]-4-(1-methyl-1,7- |
| | diazaspiro[3.4]octan-7-yl)pyridazin-3-one |
| 117 | 2-methyl-6-[5-(1-methylcyclopropoxy)-1H-indazol-3-yl]-4-(1-methyl-1,7- |
| | diazaspiro[4.4]nonan-7-yl)pyridazin-3-one |
| 118 | 4-(1-ethyl-1,7-diazaspiro[4.4]nonan-7-yl)-2-methyl-6-[5-(1-methylcyclopropoxy)- |
| | 1H-indazol-3-yl]pyridazin-3-one |
| 119 | 4-(1,1-dioxo-1,4-thiazinan-4-yl)-2-methyl-6-[5-(1-methylcyclopropoxy)-1H- |
| | indazol-3-yl]pyridazin-3-one |
| 120 | 4-[3-(chloromethyl)-3-(hydroxymethyl)pyrrolidin-1-yl]-2-methyl-6-[5-(1- |
| | methylcyclopropoxy)-1H-indazol-3-yl]pyridazin-3-one |
| 121 | 4-[(4R)-4-hydroxy-3,3-dimethyl-pyrrolidin-1-yl]-2-methyl-6-[5-(1- |
| | methylcyclopropoxy)-1H-indazol-3-yl]pyridazin-3-one |
| 122 | 4-[(4S)-4-hydroxy-3,3-dimethyl-pyrrolidin-1-yl]-2-methyl-6-[5-(1- |
| | methylcyclopropoxy)-1H-indazol-3-yl]pyridazin-3-one |
| 123 | 4-[(4S)-4-hydroxy-4-methyl-azepan-1-yl]-2-methyl-6-[5-(1-methylcyclopropoxy)- |
| | 1H-indazol-3-yl]pyridazin-3-one |
| 124 | 4-[(4R)-4-hydroxy-4-methyl-azepan-1-yl]-2-methyl-6-[5-(1-methylcyclopropoxy)- |
| | 1H-indazol-3-yl]pyridazin-3-one |
| 125 | 4-[(7R)-7-(hydroxymethyl)-1,4-oxazepan-4-yl]-2-methyl-6-[5-(1- |
| | methylcyclopropoxy)-1H-indazol-3-yl]pyridazin-3-one |
| 126 | 4-[(7S)-7-(hydroxymethyl)-1,4-oxazepan-4-yl]-2-methyl-6-[5-(1- |
| | methylcyclopropoxy)-1H-indazol-3-yl]pyridazin-3-one |
| 127 | 4-[(4S,5R)-4-hydroxy-5-methyl-azepan-1-yl]-2-methyl-6-[5-(1- |
| | methylcyclopropoxy)-1H-indazol-3-yl]pyridazin-3-one |
| 128 | 4-[(4R,5S)-4-hydroxy-5-methyl-azepan-1-yl]-2-methyl-6-[5-(1- |
| | methylcyclopropoxy)-1H-indazol-3-yl]pyridazin-3-one |
| 129 | 4-[(4S,5S)-4-hydroxy-5-methyl-azepan-1-yl]-2-methyl-6-[5-(1- |
| | methylcyclopropoxy)-1H-indazol-3-yl]pyridazin-3-one |
| 130 | 4-[(4R,5R)-4-hydroxy-5-methyl-azepan-1-yl]-2-methyl-6-[5-(1- |
| | methylcyclopropoxy)-1H-indazol-3-yl]pyridazin-3-one |
| 131 | 4-[(4R,5S)-4-fluoro-5-hydroxy-azepan-1-yl]-2-methyl-6-[5-(1- |
| | methylcyclopropoxy)-1H-indazol-3-yl]pyridazin-3-one |
| 132 | 4-[(4S,5R)-4-fluoro-5-hydroxy-azepan-1-yl]-2-methyl-6-[5-(1- |
| | methylcyclopropoxy)-1H-indazol-3-yl]pyridazin-3-one |
| 133 | 4-[(4R,5R)-4-fluoro-5-hydroxy-azepan-1-yl]-2-methyl-6-[5-(1- |
| | methylcyclopropoxy)-1H-indazol-3-yl]pyridazin-3-one |
| 134 | 4-[(4S,5S)-4-fluoro-5-hydroxy-azepan-1-yl]-2-methyl-6-[5-(1- |
| | methylcyclopropoxy)-1H-indazol-3-yl]pyridazin-3-one |

In some embodiments, the compound is a pharmaceutically acceptable salt of any one of the compounds described in Table 1.

In some embodiments, the compound is a lithium salt, sodium salt, potassium salt, calcium salt, or magnesium salt of any one of the compounds described in Table 1.

In some embodiments, the compound is a salt of any acid described in the Table 2 and any one of the compounds described in Table 1.

TABLE 2
Pharmaceutical acceptable acid forming
salts with the Compound of Formula (I).

1-hydroxy-2-naphthoic acid
2,2-dichloroacetic acid
2-hydroxyethanesulfonic acid
2-oxoglutaric acid
4-acetamidobenzoic acid
4-aminosalicylic acid
acetic acid
adipic acid
ascorbic acid (L)
aspartic acid (L)
benzenesulfonic acid
benzoic acid
camphoric acid (+)
camphor-10-sulfonic acid (+)
capric acid (decanoic acid)
caproic acid (hexanoic acid)
caprylic acid (octanoic acid)
carbonic acid
cinnamic acid
citric acid
cyclamic acid
dodecylsulfuric acid
ethane-1,2-disulfonic acid
ethanesulfonic acid
formic acid
fumaric acid
galactaric acid
gentisic acid
glucoheptonic acid (D)
gluconic acid (D)
glucuronic acid (D)
glutamic acid
glutaric acid
glycerophosphoric acid
glycolic acid
hippuric acid
hydrobromic acid
hydrochloric acid
isobutyric acid
lactic acid (DL)
lactobionic acid
lauric acid
maleic acid
malic acid (−L)
malonic acid
mandelic acid (DL)
methanesulfonic acid
naphthalene-1,5-disulfonic acid
naphthalene-2-sulfonic acid
nicotinic acid
nitric acid
oleic acid
oxalic acid
palmitic acid
pamoic acid
phosphoric acid
proprionic acid
pyroglutamic acid (−L)
salicylic acid
sebacic acid
stearic acid
succinic acid
sulfuric acid
tartaric acid (+L)
thiocyanic acid
toluenesulfonic acid (p)
undecylenic acid

In some embodiments, the compound is a salt of acetic acid and any one of the compounds described in Table 1.

In some embodiments, the compound is a salt of adipic acid and any one of the compounds described in Table 1.

In some embodiments, the compound is a salt of ascorbic acid (L) and any one of the compounds described in Table 1.

In some embodiments, the compound is a salt of hydrobromic acid and any one of the compounds described in Table 1.

In some embodiments, the compound is a salt of hydrochloric acid and any one of the compounds described in Table 1.

In some embodiments, the compound is a salt of citric acid and any one of the compounds described in Table 1.

In some embodiments, the compound is a salt of glutamic acid and any one of the compounds described in Table 1.

In some embodiments, the compound is a salt of oxalic acid and any one of the compounds described in Table 1.

In some embodiments, the compound is a salt of formic acid and any one of the compounds described in Table 1.

In some embodiments, the compound is a salt of sulfuric acid and any one of the compounds described in Table 1.

In some aspects, the present disclosure provides a compound being an isotopic derivative (e.g., isotopically labeled compound) of any one of the compounds of the Formulae disclosed herein.

In some embodiments, the compound is an isotopic derivative of any one of the compounds described in Table 1 and prodrugs and pharmaceutically acceptable salts thereof.

In some embodiments, the compound is an isotopic derivative of any one of the compounds described in Table 1 and pharmaceutically acceptable salts thereof.

In some embodiments, the compound is an isotopic derivative of any one of prodrugs of the compounds described in Table 1 and pharmaceutically acceptable salts thereof.

In some embodiments, the compound is an isotopic derivative of any one of the compounds described in Table 1.

It is understood that the isotopic derivative can be prepared using any of a variety of art-recognized techniques. For example, the isotopic derivative can generally be prepared by carrying out the procedures disclosed in the Schemes and/or in the Examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

The term “isotopic derivative”, as used herein, refers to a derivative of a compound in which one or more atoms are isotopically enriched or labelled. For example, an isotopic derivative of a compound of Formula (I) is isotopically enriched with regard to, or labelled with, one or more isotopes as compared to the corresponding compound of Formula (I). In some embodiments, the isotopic derivative is enriched with regard to, or labelled with, one or more atoms selected from ²H, ¹³C, ¹⁴C, ¹⁵N, ¹⁸O, ²⁹Si, ³¹P, and ³⁴S. In some embodiments, the isotopic derivative is a deuterium labeled compound (i.e., being enriched with ²H with regard to one or more atoms thereof).

In some embodiments, the compound is a deuterium labeled compound of any one of the compounds described in Table 1 and prodrugs and pharmaceutically acceptable salts thereof.

In some embodiments, the compound is a deuterium labeled compound of any one of the compounds described in Table 1 and pharmaceutically acceptable salts thereof.

In some embodiments, the compound is a deuterium labeled compound of any one of the prodrugs of the compounds described in Table 1 and pharmaceutically acceptable salts thereof.

In some embodiments, the compound is a deuterium labeled compound of any one of the compounds described in Table 1.

It is understood that the deuterium labeled compound comprises a deuterium atom having an abundance of deuterium that is substantially greater than the natural abundance of deuterium, which is 0.015%.

In some embodiments, the deuterium labeled compound has a deuterium enrichment factor for each deuterium atom of at least 3500 (52.5% deuterium incorporation at each deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). As used herein, the term “deuterium enrichment factor” means the ratio between the deuterium abundance and the natural abundance of a deuterium.

It is understood that the deuterium labeled compound can be prepared using any of a variety of art-recognized techniques. For example, the deuterium labeled compound can generally be prepared by carrying out the procedures disclosed in the Schemes and/or in the Examples described herein, by substituting a deuterium labeled reagent for a non-deuterium labeled reagent.

A compound of the disclosure or a pharmaceutically acceptable salt or solvate thereof that contains the aforementioned deuterium atom(s) is within the scope of the disclosure. Further, substitution with deuterium (i.e., ²H) may afford certain therapeutic advantages resulting from greater metabolic stability, e.g., increased in vivo half-life or reduced dosage requirements.

In some embodiments, the compound is a ¹⁸F labeled compound.

In some embodiments, the compound is a ¹²³I labeled compound, a ¹²⁴I labeled compound, a ¹²⁵I labeled compound, a ¹²⁹I labeled compound, a ¹³¹I labeled compound, a ¹³⁵I labeled compound, or any combination thereof.

In some embodiments, the compound is a ³³S labeled compound, a ³⁴S labeled compound, a ³⁵S labeled compound, a ³⁶S labeled compound, or any combination thereof.

It is understood that the ¹⁸F, ¹²³I, ¹²⁴I, ¹²⁵I, ¹²⁹I, ¹³¹I, ¹³⁵I, ³S, ³⁴S, ³⁵S, and/or ³⁶S labeled compound, can be prepared using any of a variety of art-recognized techniques. For example, the deuterium labeled compound can generally be prepared by carrying out the procedures disclosed in the Schemes and/or in the Examples described herein, by substituting a ¹⁸F, ¹²³I, ¹²⁴I, ¹²⁵I, ¹²⁹I, ¹³¹I, ¹³⁵I, ³S, ³⁴S, ³S, and/or ³⁶S labeled reagent for a non-isotope labeled reagent.

A compound of the disclosure or a pharmaceutically acceptable salt or solvate thereof that contains one or more of the aforementioned ¹⁸F, ¹²³I, ¹²⁴I, ¹²⁵I, ¹²⁹I, ¹³¹I, ¹³⁵I, ³S, ³⁴S, ³S, and ³⁶S atom(s) is within the scope of the disclosure. Further, substitution with isotope (e.g., ¹⁸F, ¹²³I, ¹²⁴I, ¹²⁵I, ¹²⁹I, ¹³¹I, ¹³⁵I, ³S, ³⁴S, ³S, and/or ³⁶S) may afford certain therapeutic advantages resulting from greater metabolic stability, e.g., increased in vivo half-life or reduced dosage requirements.

For the avoidance of doubt, it is to be understood that, where in this specification a group is qualified by “described herein”, the said group encompasses the first occurring and broadest definition as well as each and all of the particular definitions for that group.

The various functional groups and substituents making up the compounds of the Formula (I) are typically chosen such that the molecular weight of the compound does not exceed 1000 Daltons. More usually, the molecular weight of the compound will be less than 900, for example less than 800, or less than 750, or less than 700, or less than 650 Daltons. More conveniently, the molecular weight is less than 600 and, for example, is 550 Daltons or less, for example 500 Daltons or less, for example 450 Daltons or less.

A suitable pharmaceutically acceptable salt of a compound of the disclosure is, for example, an acid-addition salt of a compound of the disclosure, which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the disclosure which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris-(2-hydroxyethyl)amine.

It will be understood that the compounds of any one of the Formulae disclosed herein and any pharmaceutically acceptable salts thereof, comprise stereoisomers, mixtures of stereoisomers, polymorphs of all isomeric forms of said compounds.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral center” refers to a carbon atom bonded to four nonidentical substituents.

As used herein, the term “chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent, and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterised by the absolute configuration of its asymmetric centre and is described by the R- and S-sequencing rules of Cahn and Prelog, or by the manner in which the molecule rotates the plane of polarised light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centres; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centres (E- and Z-isomers). It is to be understood that the present disclosure encompasses all optical, diastereoisomers and geometric isomers and mixtures thereof that possess inflammasome inhibitory activity.

The present disclosure also encompasses compounds of the disclosure as defined herein which comprise one or more isotopic substitutions.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonamides, tetrazoles, sulfonates and phosphonates. See, e.g., Patani and LaVoie, Chem. Rev. 96, 3147-3176, 1996.

It is also to be understood that certain compounds of any one of the Formulae disclosed herein may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate. It is to be understood that the disclosure encompasses all such solvated forms that possess inflammasome inhibitory activity.

It is also to be understood that certain compounds of any one of the Formulae disclosed herein may exhibit polymorphism, and that the disclosure encompasses all such forms, or mixtures thereof, which possess inflammasome inhibitory activity. It is generally known that crystalline materials may be analysed using conventional techniques such as X-Ray Powder Diffraction analysis, Differential Scanning Calorimetry, Thermal Gravimetric Analysis, Diffuse Reflectance Infrared Fourier Transform (DRIFT) spectroscopy, Near Infrared (NIR) spectroscopy, solution and/or solid state nuclear magnetic resonance spectroscopy. The water content of such crystalline materials may be determined by Karl Fischer analysis.

Compounds of any one of the Formulae disclosed herein may exist in a number of different tautomeric forms and references to compounds of Formula (I) or (II) include all such forms. For the avoidance of doubt, where a compound can exist in one of several tautomeric forms, and only one is specifically described or shown, all others are nevertheless embraced by Formula (I) or (II). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

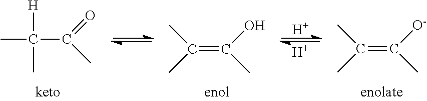

Compounds of any one of the Formulae disclosed herein containing an amine function may also form N-oxides. A reference herein to a compound of Formula (I) or (II) that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-oxides can be formed by treatment of the corresponding amine with an oxidising agent such as hydrogen peroxide or a peracid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with meta-chloroperoxybenzoic acid (mCPBA), for example, in an inert solvent such as dichloromethane.

The compounds of any one of the Formulae disclosed herein may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substituents at the ester or amide group in any one of the Formulae disclosed herein.

Accordingly, the present disclosure includes those compounds of any one of the Formulae disclosed herein as defined hereinbefore when made available by organic synthesis and when made available within the human or animal body by way of cleavage of a prodrug thereof. Accordingly, the present disclosure includes those compounds of any one of the Formulae disclosed herein that are produced by organic synthetic means and also such compounds that are produced in the human or animal body by way of metabolism of a precursor compound, that is a compound of any one of the Formulae disclosed herein may be a synthetically produced compound or a metabolically-produced compound.

A suitable pharmaceutically acceptable prodrug of a compound of any one of the Formulae disclosed herein is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of any one of the Formulae disclosed herein that possesses a hydroxy group is, for example, an in vivo cleavable ester or ether thereof. An in vivo cleavable ester or ether of a compound of any one of the Formulae disclosed herein containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl. Suitable pharmaceutically acceptable ether forming groups for a hydroxy group include a-acyloxyalkyl groups such as acetoxymethyl and pivaloyloxymethyl groups.

A suitable pharmaceutically acceptable prodrug of a compound of any one of the Formulae disclosed herein that possesses a carboxy group is, for example, an in vivo cleavable amide thereof, for example an amide formed with an amine such as ammonia, a C₁₋₄alkylamine such as methylamine, a (C₁-C₄ alkyl)₂amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of any one of the Formulae disclosed herein that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl,morpholinomethyl,piperazin-1-ylmethyl and 4-(C₁-C₄ alkyl)piperazin-1-ylmethyl.

The in vivo effects of a compound of any one of the Formulae disclosed herein may be exerted in part by one or more metabolites that are formed within the human or animal body after administration of a compound of any one of the Formulae disclosed herein. As stated hereinbefore, the in vivo effects of a compound of any one of the Formulae disclosed herein may also be exerted by way of metabolism of a precursor compound (a prodrug).

### Method of Synthesizing the Compounds

The compounds of the present invention may be made by a variety of methods, including standard chemistry. Suitable synthetic routes are depicted in the Schemes given below.

The compounds of Formula (I) may be prepared by methods known in the art of organic synthesis as set forth in part by the following synthetic schemes. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of those skilled in the art will recognize if a stereocenter exists in the compounds of Formula (I). Accordingly, the present invention includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds described herein may be made from commercially available starting materials or synthesized using known organic, inorganic, and/or enzymatic processes.

### Preparation of Compounds

The compounds of the present invention can be prepared in a number of ways well known to those skilled in the art of organic synthesis. By way of example, compounds of the present invention can be synthesized using the methods described below, together with synthetic methods known in the art of synthetic organic chemistry, or variations thereon as appreciated by those skilled in the art. Suitable methods include but are not limited to those methods described below. Compounds of the present invention can be synthesized by following the steps outlined in General Procedures A or B which comprise different sequences of assembling intermediates or compounds. Starting materials are either commercially available or made by known procedures in the reported literature or as illustrated below.

### General Procedure A

In general, the compound of the Formula (I-I) can be prepared using sequences of reactions presented at the Scheme 1:

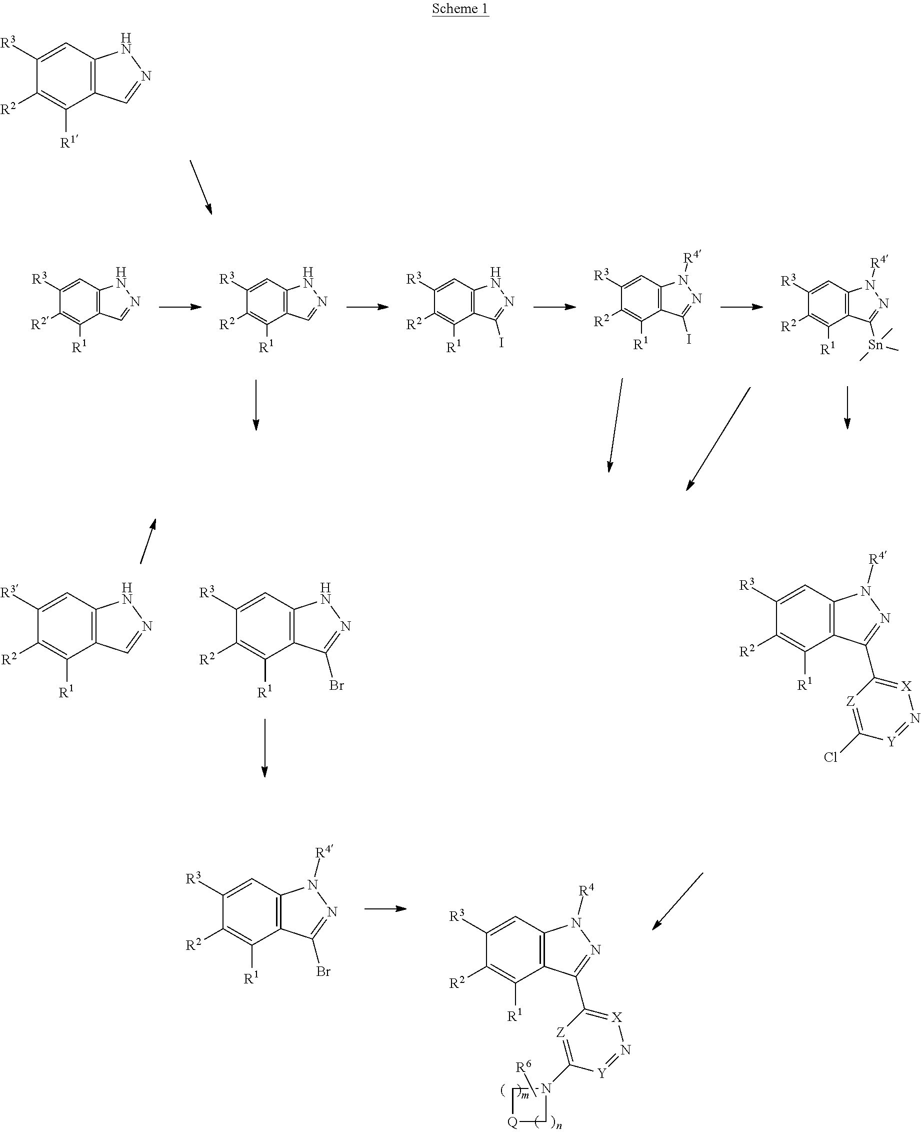

All reagents may be commercially available compounds itself or products of synthesis from commercially available reagents. For preparation of these reagents may be used one step or multistep synthetic procedures, including but not limited procedures described herein in preparative part.

### General Procedure B

In general, the compound of the Formula (I-II) can be prepared using sequences of reactions presented at the Scheme 2:

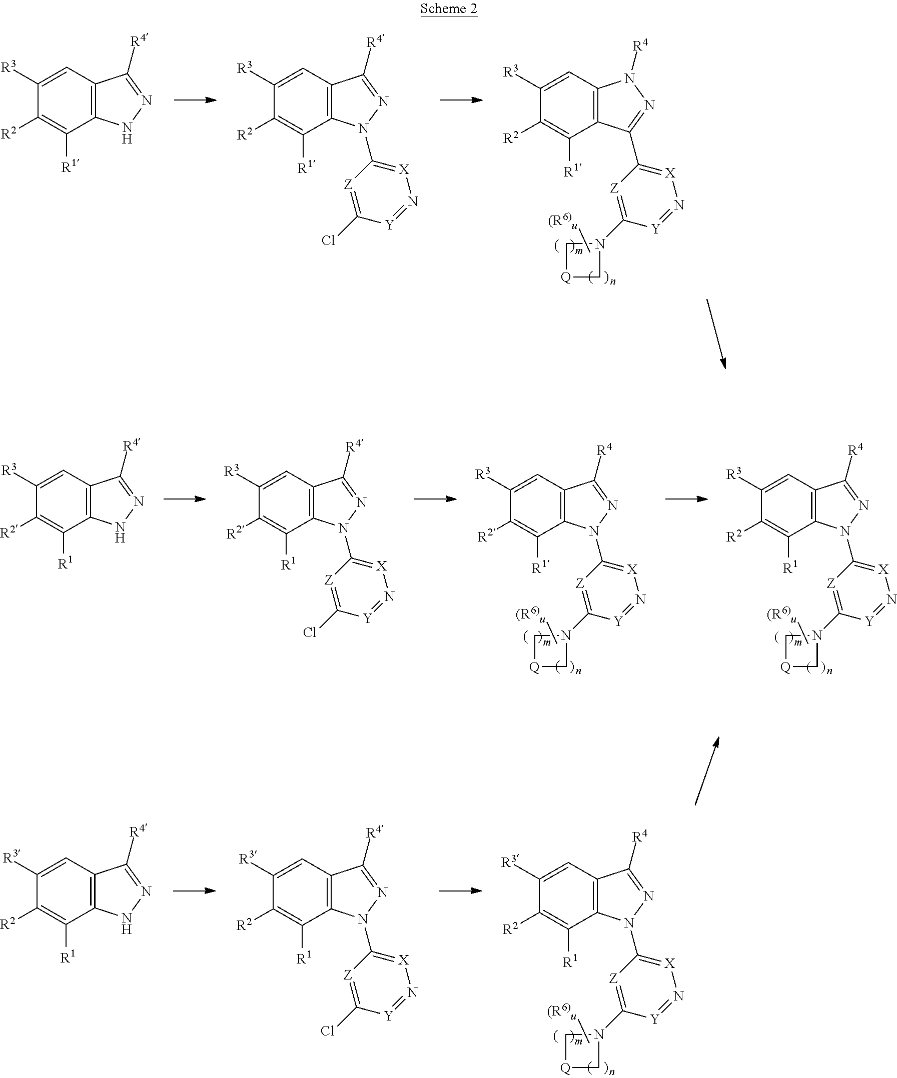

As a specific non-limiting example of this procedure can be presented by the reaction of preparation of the compound of Formula I-II in case when R^{1′}, R^{2′} or R^{3′} is Br.

All reagents may be commercially available compounds itself or products of synthesis from commercially available reagents. For preparation these reagents may be used one step or multistep synthetic procedures, including but not limited procedures described herein in preparative part.

### General Procedure C

In general, the compound of the Formula (I-II) can be prepared using sequences of reactions presented at the Scheme 3:

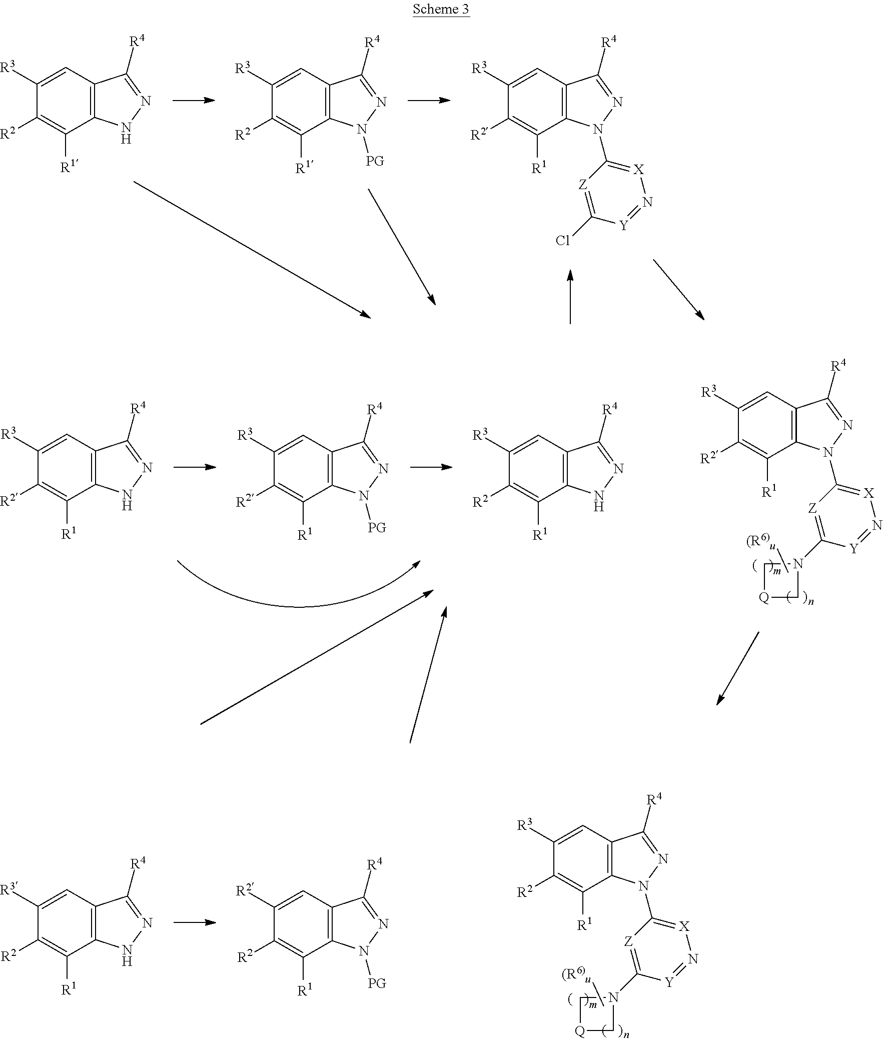

wherein PG is appropriate protecting group.

All reagents may be commercially available compounds itself or products of synthesis from commercially available reagents. For preparation these reagents may be used one step or multistep synthetic procedures, including but not limited procedures described herein in preparative part.

It should be obvious for specialist in this field that any of compound of Formula (I) obtained according to the procedures A, B, or C described above may be a subject for further transformation and modification that will led to obtain other compound of Formula (I).

### Biological Assays

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

### Pharmaceutical Compositions

In some aspects, the present disclosure provides a pharmaceutical composition comprising a compound of the present disclosure as an active ingredient. In some embodiments, the present disclosure provides a pharmaceutical composition comprising at least one compound of each of the formulae described herein, or a pharmaceutically acceptable salt or solvate thereof, and one or more pharmaceutically acceptable carriers or excipients. In some embodiments, the present disclosure provides a pharmaceutical composition comprising at least one compound selected from Table 1.

As used herein, the term “composition” is intended to encompass a product comprising the specified ingredients in the specified amounts, as well as any product which results, directly or indirectly, from combination of the specified ingredients in the specified amounts.

Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component, colorants, flavors, stabilizers, buffers, artificial and natural sweeteners, dispersants, thickeners, solubilizing agents, and the like.

Liquid formulations also are suitable for oral administration include liquid formulation including emulsions, syrups, elixirs, aqueous solutions, aqueous suspensions. These include solid form preparations which are intended to be converted to liquid form preparations shortly before use. Emulsions may be prepared in solutions, for example, in aqueous propylene glycol solutions or may contain emulsifying agents such as lecithin, sorbitan monooleate, or acacia. Aqueous solutions can be prepared by dissolving the active component in water and adding suitable colorants, flavors, stabilizing, and thickening agents. Aqueous suspensions can be prepared by dispersing the finely divided active component in water with viscous material, such as natural or synthetic gums, resins, methylcellulose, sodium carboxymethylcellulose, and other well-known suspending agents.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilization from solution for constitution before use with a suitable vehicle, e.g., sterile, pyrogen-free water.

The compounds of present disclosure can be formulated for oral administration in forms such as tablets, capsules (each of which includes sustained release or timed release formulations), pills, powders, granules, elixirs, tinctures, suspensions, syrups and emulsions. The compounds of present disclosure on can also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-o-cyclodextrin, methyl-o-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio)propyl-β-cyclodextrin, glucosyl-β-cyclodextrin, sulfated β-cyclodextrin (S-β-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

In some embodiments, examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof. In some embodiments, the tonicity agent is selected from the group consisting of a glycol (such as propylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols—such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base—depending on the formulation, the addition of one of the acid or base may be sufficient to bring the mixture to the desired pH range.

The aqueous vehicle may also contain a buffering agent to stabilize the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and F-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

According to a further aspect of the disclosure there is provided a pharmaceutical composition which comprises a compound of the disclosure as defined hereinbefore, or a pharmaceutically acceptable salt, hydrate or solvate thereof, in association with a pharmaceutically acceptable diluent or carrier.

In some embodiments, a pharmaceutical composition described herein may further comprise one or more additional pharmaceutically active agents.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more coloring, sweetening, flavoring and/or preservative agents.

A therapeutically effective amount of a compound of the present disclosure for use in therapy is an amount sufficient to treat or prevent a LRRK2 related condition referred to herein, slow its progression and/or reduce the symptoms associated with the condition.

A therapeutically effective amount of a compound of the present disclosure for use in therapy is an amount sufficient to treat an LRRK2 related condition referred to herein, slow its progression and/or reduce the symptoms associated with the condition.

The size of the dose for therapeutic or prophylactic purposes of a compound of Formula (I) will naturally vary according to the nature and severity of the conditions, the age and sex of the animal or subject and the route of administration, according to well-known principles of medicine.

### Methods of Use

In some aspects, the present disclosure provides a method of inhibiting of LRRK2 (e.g., in vitro or in vivo), comprising contacting a cell with a therapeutically effective amount of a compound of the present disclosure or a pharmaceutically acceptable salt thereof.

In some aspects, the present disclosure provides a method of treating or preventing a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some embodiments, the disease or disorder is associated with LRRK2. In some embodiments, the disease or disorder is a disease or disorder in which LRRK2 is implicated.

The compounds of the invention are also useful in treating diseases associated with LRRK2. For example, diseases and conditions treatable according to the methods of the invention include Parkinson Disease 8, Autosomal Dominant (PARK8); Hereditary Late-Onset Parkinson Disease (LOPD); Spinocerebellar Atrophy; Klippel-Feil Syndrome 1, Autosomal Dominant (KFS1); Autosomal Dominant Cerebellar Ataxia (SCA); Parkinson Disease, Late-Onset (PD); Parkinson Disease 2, Autosomal Recessive Juvenile (PARK2); Parkinsonism; Rem Sleep Behavior Disorder; Dementia, Lewy Body (DLB); Lrrk2 Parkinson Disease; Parkinson Disease 3, Autosomal Dominant (PARK3); Early-Onset Parkinson's Disease; Multiple System Atrophy 1 (MSA1); Essential Tremor; Movement Disease; Supranuclear Palsy, Progressive, 1 (PSNP1); Klippel-Feil Syndrome 1; Dementia; Parkinson Disease 10 (PARK10); Tremor; Frontotemporal Dementia (FTD); Postencephalitic Parkinson Disease; Vascular Parkinsonism; Aphasia; Parkinson Disease 1, Autosomal Dominant (PARK1); Athetosis; Klippel-Feil Syndrome (KFS); Kufor-Rakeb Syndrome (KRS); Leprosy 3 (LPRS3); Alzheimer Disease 8 (AD8); Crohn's Disease; Rheumatoid Arthritis (RA); Alzheimer Disease (AD); Color Agnosia; Gaucher Disease, Type I (GD1); Parkinson Disease 15, Autosomal Recessive Early-Onset (PARK15); Von Economo's Disease; Gerstmann-Straussler Disease (GSD); Amyotrophic Lateral Sclerosis-Parkinsonism/dementia Complex 1 (ALS-PDC1); Dystonia; Sphingolipidosis; Radial Nerve Lesion; Toxic Encephalopathy; Sleep Disorder; Pick Disease of Brain (PIDB); Ophthalmomyiasis; Gaucher's Disease (GD); Optic Atrophy 7 with or Without Auditory Neuropathy (OPA7); Ulnar Nerve Lesion; Inflammatory Bowel Disease 4 (IBD4); Inflammatory Bowel Disease; 3-Methylglutaconic Aciduria, Type Iii (MGCA3); Nervous System Disease; Amyotrophic Lateral Sclerosis 1 (ALS1); Mitochondrial Complex I Deficiency, Nuclear Type 1 (MC1DN1).

In some embodiments, the disease or disorder is a Parkinson Disease (PD).

In some aspects, the present disclosure provides a method of treating or preventing a Parkinson Disease (PD) in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a Parkinson Disease (PD) in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a compound of the present disclosure or a pharmaceutically acceptable salt thereof for use in inhibiting of LRRK2 (e.g., in vitro or in vivo).

In some aspects, the present disclosure provides a compound of the present disclosure or a pharmaceutically acceptable salt thereof for use in treating or preventing a disease or disorder disclosed herein.

In some aspects, the present disclosure provides a compound of the present disclosure or a pharmaceutically acceptable salt thereof for use in treating a disease or disorder disclosed herein.

In some aspects, the present disclosure provides a compound of the present disclosure or a pharmaceutically acceptable salt thereof for use in treating or preventing a Parkinson Disease (PD) in a subject in need thereof.

In some aspects, the present disclosure provides a compound of the present disclosure or a pharmaceutically acceptable salt thereof for use in treating Parkinson Disease (PD) in a subject in need thereof.

In some aspects, the present disclosure provides use of a compound of the present disclosure or a pharmaceutically acceptable salt thereof in the manufacture of a medicament for inhibiting of LRRK2 (e.g., in vitro or in vivo).

In some aspects, the present disclosure provides use of a compound of the present disclosure or a pharmaceutically acceptable salt thereof in the manufacture of a medicament for treating or preventing a disease or disorder disclosed herein.

In some aspects, the present disclosure provides use of a compound of the present disclosure or a pharmaceutically acceptable salt thereof in the manufacture of a medicament for treating a disease or disorder disclosed herein.

In some aspects, the present disclosure provides use of a compound of the present disclosure or a pharmaceutically acceptable salt thereof in the manufacture of a medicament for treating or preventing Parkinson Disease (PD) in a subject in need thereof.

In some aspects, the present disclosure provides use of a compound of the present disclosure or a pharmaceutically acceptable salt thereof in the manufacture of a medicament for treating Parkinson Disease (PD) in a subject in need thereof.

The present disclosure provides compounds that function as inhibitors of LRRK2 (e.g., in vitro or in vivo). The present disclosure therefore provides a method of inhibiting of LRRK2 in vitro or in vivo, said method comprising contacting a cell with a therapeutically effective amount of a compound, or a pharmaceutically acceptable salt thereof, as defined herein.

In some embodiments, the inhibitor of LRRK2 is a compound of the present disclosure.

Effectiveness of compounds of the disclosure can be determined by industry-accepted assays/disease models according to standard practices of elucidating the same as described in the art and are found in the current general knowledge.

The present disclosure also provides a method of treating a disease or disorder in which LRRK2 is implicated in a subject in need of such treatment, said method comprising administering to said subject a therapeutically effective amount of a compound, or a pharmaceutically acceptable salt thereof, or a pharmaceutical composition as defined herein.

In some embodiments, the subject is a mammal. In some embodiments, the subject is a human.

### Routes of Administration

The compounds of the disclosure or pharmaceutical compositions comprising these compounds may be administered to a subject by any convenient route of administration, whether systemically/peripherally or topically (i.e., at the site of desired action).

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### EXAMPLES

### General Synthetical Procedures and Examples of the Compound's Preparation.

All reagents were commercial and were used without further purification. Yields refer to purified and spectroscopically pure compounds. Thin layer chromatography (TLC) was performed using Merck TLC Aluminum sheets silica gel 60 F₂₅₄ plates and visualized by fluorescence quenching under UV light. Flash chromatography was performed using silica gel (Chromatorex, MB 70-40/75, 40-75 μm) purchased by Fuji Silysia Chemicals. NMR spectra were recorded on a Varian-400MR operating at 400 MHz for ¹H. Chemical shifts are reported in ppm with the solvent resonance as the internal standard. Data is reported as follows: s=singlet, br=broad, d=doublet, t=triplet, q=quartet, m=multiplet, dd=doublet of doublets; coupling constants in Hz; integration. The purities were recorded on Waters e2695 separations Module/2998 PDA Detector HPLC system (Column: XBridge C18, 5 μm, 4.6 mm (ID)×150 mm (L), Eluent: the mixture of mobile phase A and B, mobile phase A: 100% acetonitrile; mobile phase B: pure water containing 0.1% formic acid and 10 mM NH₄OAc, Flow rate: 0.5 mL/min. detection: UV, 254 nm)

Abbreviations used in the following examples and elsewhere herein are:
AcOH acetic acid
anh. anhydrous
aq. aqueous
br. broad
BSA bovine serum albumin
CPME cyclopentyl methyl ether
d duplet
DCM dichloromethane
DME Dimethoxyethane
DMEM Dulbecco's modified Eagle's medium
DMF N,N-dimethyl formamide
DMSO dimethyl sulfoxide
DTT dithiothreitol
FBS fetal bovine serum
h hour(s)
HPLC high pressure (or performance) liquid chromatography
LCMS liquid chromatography mass spectrometry
LiHMDS lithium bis(trimethylsilyl)amide
m multiplet
M molar
MHz megahertz
min minutes
Ms Mesyl
NBS N-bromosuccinimide
NMR nuclear magnetic resonance
Pd₂(dba)₃ tris(dibenzylideneacetone)dipalladium(0)
q quadruplet
rt room temperature
s singlet
s solid
SEMCl 2-(trimethylsilyl)ethoxymethyl chloride
t temperature, triplet
TFA trifluoroacetic acid
THF tetrahydrofuran
TLC thin layer chromatography
TrCl trityl chloride
TRIS 2-amino-2-(hydroximethyl)propane-1,3-diol
XantPhos 4,5-Bis(diphenylphosphino)-9,9-dimethylxanthene

### Synthesis of Building Blocks

### Synthesis of 5-bromo-3-chloropyridazine (P2)

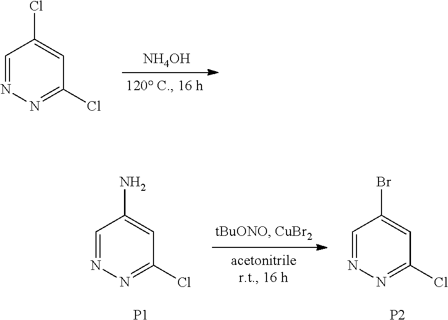

Preparation 1. Methyl 5-bromo-2-[(tert-butoxycarbonyl)amino]benzoate (P1). In a sealed tube, a mixture of 3,5-dichloropyridazine (3.4 g, 22.82 mmol, 1.0 eq) and 30% ammonium hydroxide (34 mL) was stirred at 120° C. for 16 h. The resulting precipitate was collected by filtration and washed with water and diethyl ether to give P1 (2.54 g, 86% yield) as a brown solid. ¹H NMR (400 MHz, DMSO-d₆), δ: 8.49 (d, J=2.4 Hz, 1H), 6.82 (br. s, 2H), 6.65 (d, J=2.4 Hz, 1H).

### Preparation 2. 5-bromo-3-chloropyridazine (P2)

To a solution of P1 (2.92 g, 22.54 mmol, 1.0 eq) and tert-butyl nitrite (4.02 mL, 33.81 mmol, 1.5 eq) in acetonitrile (60 mL) was added copper(II) bromide (7.55 g, 33.81 mmol, 1.5 eq) at 0° C. After the reaction mixture was stirred at rt for 16 h, the reaction mixture was treated with water and extracted with ethyl acetate for three times. The combined organic layers were dried over Na₂SO₄₍ₛ₎, filtered, and concentrated in vacuo. The residue was purified by silica gel column chromatography (n-hexane:EtOAc=5:1) to give P2 (2.04 g, 47% yield) as a yellow solid. H NMR (400 MHz, CDCl₃), δ: 9.22 (d, J=2.0 Hz, 1H), 7.78 (d, J=2.0 Hz, 1H).

### Synthesis of 1-(5-(5-isopropoxy-1-trityl-1H-indazol-3-yl)pyridazin-3-yl)azetidin-3-ol (P8)

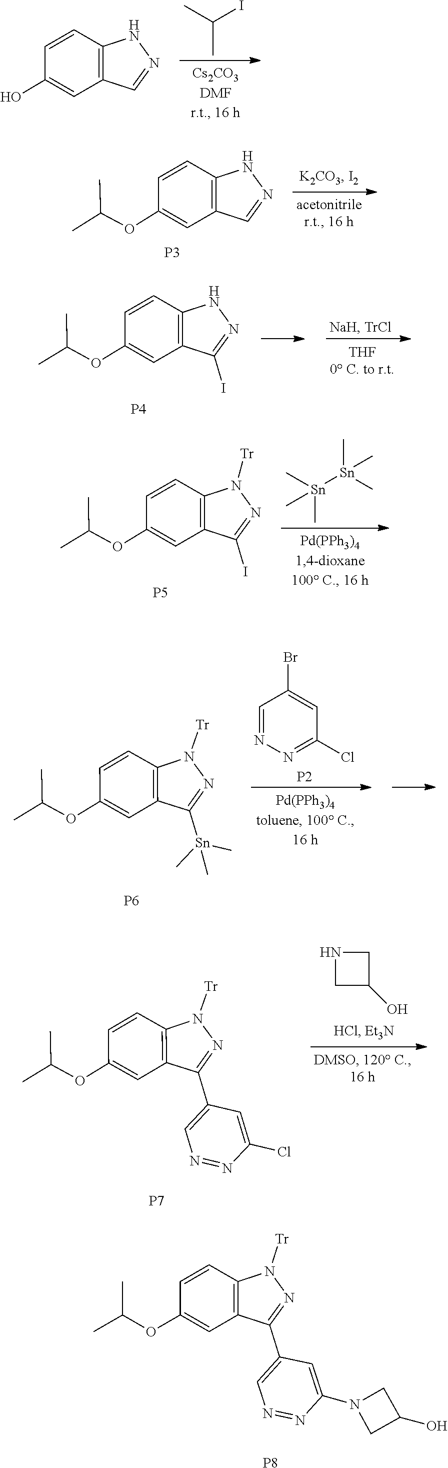

### Preparation 3. 5-isopropoxy-1H-indazole (P3)

To a solution of 1H-indazol-5-ol (1.00 g, 7.46 mmol, 1.0 eq) in DMF (10 mL) was added cesium carbonate (2.91 g, 8.95 mmol, 1.2 eq). The reaction mixture was stirred at rt for 15 min. 2-Iodopropane (0.89 mL, 8.95 mmol, 1.2 eq) was added to the reaction, and the mixture was stirred at rt for 16 h. The mixture was treated with water, extracted with ethyl acetate for three times. The combined organic layers were dried over Na₂SO₄₍ₛ₎, filtered, and concentrated in vacuo to give a residue, which was purified by silica gel column chromatography (n-hexane:EtOAc=3:1) to give P3 (876 mg, 67% yield) as an orange solid. ¹H NMR (400 MHz, DMSO-d₆), δ: 12.86 (br. s, 1H), 7.92-7.90 (m, 1H), 7.41 (d, J=9.0 Hz, 1H), 7.17 (d, J=2.2 Hz, 1H), 6.96 (dd, J=9.0, 2.2 Hz, 1H), 4.56 (septet, J=6.0 Hz, 1H), 1.27 (d, J=6.0 Hz, 6H).

### Preparation 4. 3-iodo-5-isopropoxy-1H-indazole (P4)

To a solution of P3 (872 mg, 4.95 mmol, 1.0 eq) in acetonitrile (21 mL) was added potassium carbonate (1.37 g, 9.90 mmol, 2.0 eq) and iodine (1.88 g, 7.42 mmol, 1.5 eq). After the solution was stirred at rt for 16 h, the mixture was quenched by saturated Na₂S₂O_{3(aq)} and extracted with ethyl acetate for three times. The combined organic layers were dried over Na₂SO₄₍ₛ₎, filtered, and concentrated in vacuo to give the residue, which was purified by silica gel column chromatography (n-hexane:EtOAc=5:1) to give P4 (1.48 g, 99% yield) as an orange foam. ¹H NMR (400 MHz, DMSO-d₆), δ: 13.34 (br. s, 1H), 7.45 (d, J=8.8 Hz, 1H), 7.05 (dd, J=8.8, 2.0 Hz, 1H), 6.75 (d, J=2.0 Hz, 1H), 4.63 (septet, J=6.0 Hz, 1H), 1.28 (d, J=6.0 Hz, 6H).

### Preparation 5. 3-iodo-5-isopropoxy-1-trityl-1H-indazole (P5)

To a solution of 60% sodium hydride in mineral oil (235.12 mg, 5.88 mmol, 1.2 eq) in anhydrous THF (10 mL) was added a solution of P4 (1.48 g, 4.90 mmol, 1.0 eq) in anhydrous THF (11 mL) at 0° C. The reaction mixture was stirred at 0° C. for 30 min, and then TrCl (1.50 g, 5.39 mmol, 1.1 eq) was added in one portion. The reaction mixture was slowly warmed up and stirred for 16 h at rt. The reaction was quenched by water and extracted with diethyl ether for three times. The combined organic layers were dried over Na₂SO₄₍ₛ₎, filtered and concentrated in vacuo to give the residue, which was purified by silica gel column chromatography (n-hexane:EtOAc=6:1) to give P5 (2.56 g, 96% yield) as an orange solid. ¹H NMR (400 MHz, CDCl₃), δ: 7.32-7.28 (m, 2H), 7.28-7.26 (m, 3H), 7.26-7.24 (m, 5H), 7.22-7.19 (m, 6H), 6.74 (d, J=2.6 Hz, 1H), 6.65 (dd, J=9.4, 2.6 Hz, 1H), 6.22 (d, J=9.4 Hz, 1H), 4.55 (septet, J=6.0 Hz, 1H), 1.34 (d, J=6.0 Hz, 6H).

### Preparation 6. 5-isopropoxy-3-(trimethylstannyl)-1-trityl-1H-indazole (P6)

A solution of P5 (1.55 g, 2.85 mmol, 1.0 eq) and hexamethyldistannane (0.86 mL, 4.27 mmol, 1.5 eq) in 1,4-dioxane (26 mL) was degassed by argon for 10 min. To the mixture was added tetrakis(triphenylphosphine)palladium(0) (263.2 mg, 0.228 mmol, 0.08 eq). The reaction mixture was stirred at 100° C. for 16 h. The solution was cooled to rt, treated with water, and extracted with ethyl acetate for three times. The combined organic layers were dried over Na₂SO₄₍ₛ₎, filtered, and concentrated in vacuo to give P6 as a product which was used in next step without further purification. ¹H NMR (400 MHz, CDCl₃), δ: 7.35-7.29 (m, 5H), 7.25-7.22 (m, 7H), 7.19-7.16 (m, 4H), 7.01 (d, J=2.4 Hz, 1H), 6.59 (dd, J=9.2, 2.4 Hz, 1H), 6.21 (d, J=9.2 Hz, 1H), 4.46 (septet, J=6.0 Hz, 1H), 1.33 (d, J=6.0 Hz, 6H), 0.35 (s, 9H).

### Preparation 7. 3-(6-chloropyridazin-4-yl)-5-isopropoxy-1-trityl-1H-indazole (P7)

A solution of crude P6 (2.85 mmol, 1.0 eq) and 5-bromo-3-chloropyridazine (P2, 991.23 mg, 5.12 mmol, 1.8 eq) in anhydrous toluene (70 mL) was degassed by argon for 10 min. To the mixture was added tetrakis(triphenylphosphine)palladium(0) (263.2 mg, 0.228 mmol, 0.08 eq) and then the reaction mixture was stirred at 110° C. for 16 h. The solution was cooled to rt, treated with water, and extracted with ethyl acetate for three times. The combined organic layers were dried over Na₂SO₄₍ₛ₎, filtered, and concentrated in vacuo to give a residue. The residue was purified by silica gel column chromatography (n-hexane:EtOAc=20:1 to 10:1) to give P7 (660 mg, 44% yield) as a yellow solid. ¹H NMR (400 MHz, CDCl₃), δ: 9.64 (d, J=1.8 Hz, 1H), 7.87 (d, J=1.8 Hz, 1H), 7.32-7.28 (m, 10H), 7.19-7.16 (m, 6H), 6.72 (dd, J=9.4, 2.4 Hz, 1H), 6.40 (d, J=9.4 Hz, 1H), 4.58 (septet, J=6.0 Hz, 1H), 1.37 (d, J=6.0 Hz, 6H).

### Preparation 8. 3-(6-chloropyridazin-4-yl)-5-isopropoxy-1-trityl-1H-indazole (P8)

To a solution of P7 (120.0 mg, 0.226 mmol, 1.0 eq) in DMSO (1.1 mL) was added triethylamine (0.19 mL, 1.36 mmol, 6.0 eq) and 3-hydroxyazetidine hydrochloride (74.3 mg, 0.678 mmol, 3.0 eq). The reaction mixture was stirred at 120° C. for 16 h. The solution was cooled to rt, treated with water and extracted with ethyl acetate for three times. The combined organic layers were dried over Na₂SO₄₍ₛ₎, filtered, and concentrated in vacuo to give the residue, which was purified by silica gel column chromatography (dichloromethane:MeOH=30:1) to give P8 (47 mg, 37% yield) as a yellow solid. ¹H NMR (400 MHz, CDCl₃), δ: 9.15 (d, J=1.6 Hz, 1H), 7.30-7.27 (m, 10H), 7.21-7.17 (m, 6H), 6.94 (d, J=2.2 Hz, 1H), 6.68 (dd, J=9.2, 2.2 Hz, 1H), 6.35 (d, J=9.2 Hz, 1H), 4.89-4.86 (m, 1H), 4.54 (septet, J=6.0 Hz, 1H), 4.47-4.43 (m, 2H), 4.05-4.01 (m, 2H), 1.35 (d, J=6.0 Hz, 6H).

### Synthesis of (R)-1-(5-(5-isopropoxy-1-trityl-1H-indazol-3-yl)pyridazin-3-yl)pyrrolidin-3-ol (P9)

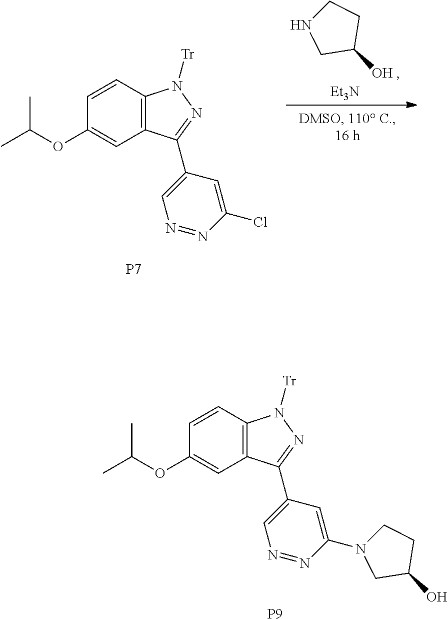

### Preparation 9. (R)-1-(5-(5-isopropoxy-1-trityl-1H-indazol-3-yl)pyridazin-3-yl)pyrrolidin-3-ol (P9)

To a solution of P7 (120.0 mg, 0.23 mmol, 1.0 eq) in DMSO (1.1 mL) was added triethylamine (0.2 mL, 1.36 mmol, 6.0 eq) and (R)-pyrrolidin-3-ol (0.055 mL, 0.68 mmol, 3.0 eq). The reaction mixture was stirred at 110° C. for 16 h. The solution was cooled to rt, treated with water, and extracted with ethyl acetate for three times. The combined organic layers were dried over Na₂SO₄₍ₛ₎, filtered, and concentrated in vacuo to give the residue, which was purified by C-18 reversed-phase column chromatography (mobile phase A: water (with NH₄HCO₃), mobile phase B: methanol, UV: 214 and 254 nm, Flow rate: 30 mL/min, Gradient: 3-90% (% B)) to give P9 (113 mg, 86% yield) as a light-yellow solid. ¹H NMR (400 MHz, CDCl₃), δ: 9.07 (d, J=1.8 Hz, 1H), 7.32-7.24 (m, 10H), 7.20-7.14 (m, 6H), 7.03 (d, J=1.8 Hz, 1H), 6.66 (dd, J=9.2, 2.4 Hz, 1H), 6.33 (d, J=9.2 Hz, 1H), 4.69-4.65 (m, 1H), 4.52 (septet, J=6.0 Hz, 1H), 3.78-3.63 (m, 4H), 2.23-2.11 (m, 2H), 1.33 (d, J=6.0 Hz, 6H).

### Synthesis of 5-isopropoxy-3-(6-(3-methylpyrrolidin-1-yl)pyridazin-4-yl)-1-trityl-1H-indazole (P10)

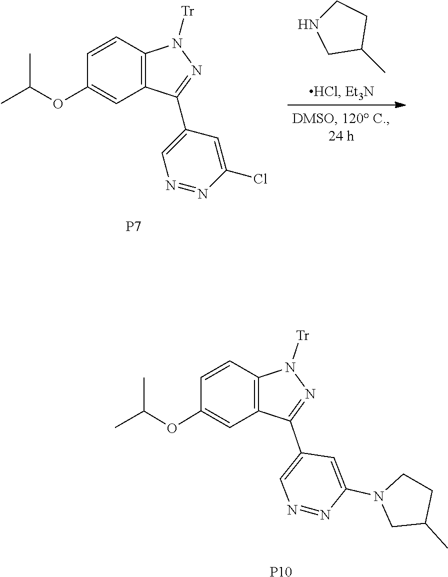

### Preparation 10. 5-isopropoxy-3-(6-(3-methylpyrrolidin-1-yl)pyridazin-4-yl)-1-trityl-1H-indazole (P10)

To a solution of P7 (120.0 mg, 0.23 mmol, 1.0 eq) in DMSO (1.1 mL) was added triethylamine (0.2 mL, 1.36 mmol, 6.0 eq) and 3-methylpyrrolidine hydrochloride (82.4 mg, 0.678 mmol, 3.0 eq). The reaction mixture was stirred at 120° C. for 24 h. The solution was cooled to rt, treated with water and extracted with ethyl acetate for three times. The combined organic layers were dried over Na₂SO₄₍ₛ₎, filtered, and concentrated in vacuo to give the residue, which was purified by silica gel column chromatography (n-hexane:EtOAc=2:1) to give P10 (66 mg, 50% yield) as a yellow solid. ¹H NMR (400 MHz, CDCl₃), δ: 9.06 (d, J=1.8 Hz, 1H), 7.33 (d, J=2.2 Hz, 1H), 7.31-7.27 (m, 8H), 7.22-7.16 (m, 7H), 7.01 (d, J=1.8 Hz, 1H), 6.68 (dd, J=9.2, 2.2 Hz, 1H), 6.34 (d, J=9.2 Hz, 1H), 4.54 (septet, J=6.0 Hz, 1H), 3.81-3.74 (m, 1H), 3.73-3.66 (m, 1H), 3.57-3.50 (m, 1H), 3.15-3.09 (m, 1H), 2.49-2.40 (m, 1H), 2.23-2.15 (m, 1H), 1.72-1.66 (m, 1H), 1.35 (d, J=6.0 Hz, 6H), 1.17 (d, J=6.8 Hz, 3H).

### Synthesis of 4-(5-(5-isopropoxy-1-trityl-1H-indazol-3-yl)pyridazin-3-yl)morpholine (P11)

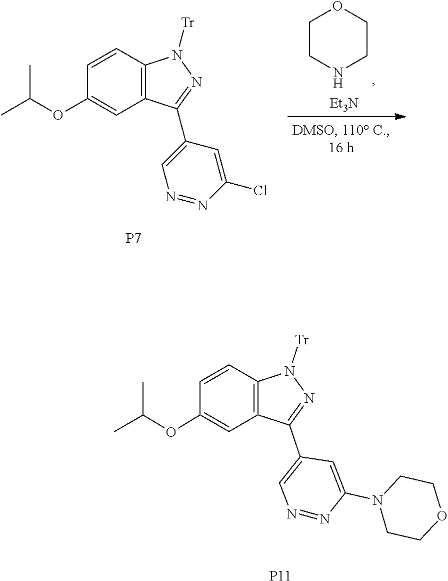

### Preparation 11. 4-(5-(5-isopropoxy-1-trityl-1H-indazol-3-yl)pyridazin-3-yl)morpholine (P11)

To a solution of P7 (50.0 mg, 0.094 mmol, 1.0 eq) in DMSO (0.50 mL) was added triethylamine (0.079 mL, 0.56 mmol, 6.0 eq) and morpholine (0.025 mg, 0.28 mmol, 3.0 eq). The reaction mixture was stirred at 110° C. for 16 h. The solution was cooled to rt, treated with water, and extracted with ethyl acetate for three times. The combined organic layers were dried over Na₂SO₄₍ₛ₎, filtered, and concentrated in vacuo to give the residue, which was purified by silica gel column chromatography (n-hexane:EtOAc=2:1) to give P11 (40 mg, 73% yield) as a yellow solid. ¹H NMR (400 MHz, CDCl₃), δ: 9.19 (d, J=1.6 Hz, 1H), 7.34-7.27 (m, 10H), 7.22-7.15 (m, 7H), 6.69 (dd, J=9.2, 2.4 Hz, 1H), 6.36 (d, J=9.6 Hz, 1H), 4.55 (septet, J=6.0 Hz, 1H), 3.90-3.85 (m, 4H), 3.68-3.64 (m, 4H), 1.35 (d, J=6.0 Hz, 6H).

### Synthesis of 5-isopropoxy-3-(6-(pyrrolidin-1-yl)pyridazin-4-yl)-1-trityl-1H-indazole (P12)

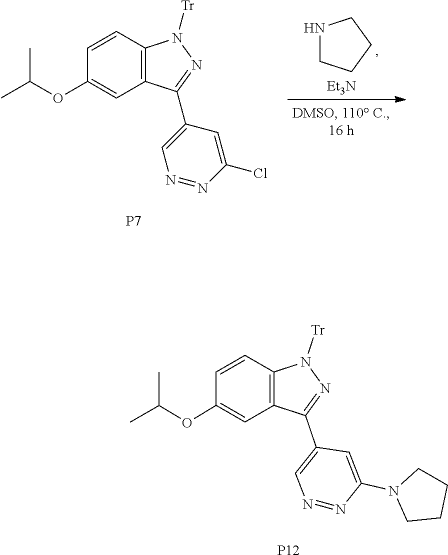

### Preparation 12. 5-isopropoxy-3-(6-(pyrrolidin-1-yl)pyridazin-4-yl)-1-trityl-1H-indazole (P12)

To a solution of P7 (240.0 mg, 0.45 mmol, 1.0 eq) in DMSO (2.0 mL) was added triethylamine (0.4 mL, 2.71 mmol, 6.0 eq) and pyrrolidine (0.110 mL, 1.36 mmol, 3.0 eq). The reaction mixture was stirred at 110° C. for 16 h. The solution was cooled to rt, treated with water, and extracted with ethyl acetate for three times. The combined organic layers were dried over Na₂SO₄₍ₛ₎, filtered, and concentrated in vacuo to give the residue, which was purified by silica gel column chromatography (n-hexane:EtOAc=1:1) to give P12 (205 mg, 80% yield) as a yellow solid. ¹H NMR (400 MHz, CDCl₃), δ: 9.07 (s, 1H), 7.34-7.29 (m, 6H), 7.26-7.15 (m, 10H), 7.04 (s, 1H), 6.68 (d, J=9.4 Hz, 1H), 6.35 (d, J=9.4 Hz, 1H), 4.54 (septet, J=6.0 Hz, 1H), 3.58 (br. m, 4H), 2.10-2.04 (m, 4H), 1.35 (d, J=6.0 Hz, 6H).

### Synthesis of 4-(6-chloro-3-methylpyridazin-4-yl)morpholine (P16)

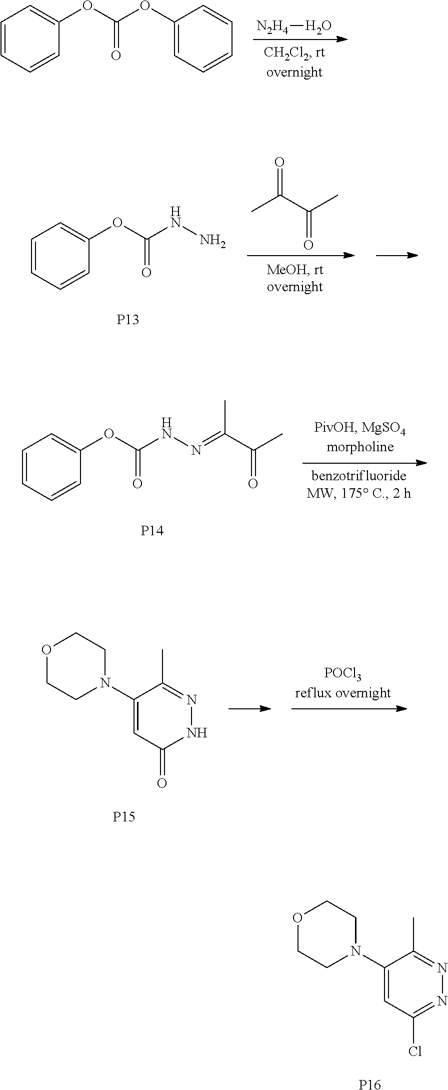

### Preparation 13. phenyl hydrazinecarboxylate (P13)

To a solution of diphenyl carbonate (15.0 g, 70.0 mmol) in DCM (230 mL) was added hydrazine hydrate (6.80 g, 140 mmol) and it was stirred at rt overnight. The reaction was concentrated to remove the solvent. The residue was purified by silica gel chromatography (0-100% ethyl acetate in n-hexane) to give P13 (5.5 g, 52%) as a white solid. ¹H NMR (400 MHz, DMSO-d₆), δ: 8.73 (br. s, 1H), 7.38-7.34 (m, 2H), 7.21-7.16 (m, 1H), 7.06 (d, J=7.6 Hz, 2H), 4.21 (br. s, 2H).

### Preparation 14. phenyl (E)-2-(3-oxobutan-2-ylidene)hydrazine-1-carboxylate (P14)

To a solution of P13 (5.5 g, 36.1 mmol) in methanol (120 mL) was added 2,3-butanedione (3.40 g, 39.7 mmol) and the reaction mixture was stirred at rt overnight. The reaction was concentrated to give the crude product P14 (7.9 g, 99%) as white solid. ¹H NMR (400 MHz, DMSO-d₆), δ: 11.13 (br. s, 1H), 7.46-7.42 (m, 2H), 7.30-7.23 (m, 2H), 2.32 (s, 3H), 1.96 (s, 3H).

### Preparation 15. 6-methyl-5-morpholinopyridazin-3(2H)-one (P15)

To a solution of P14 (1.0 g, 4.5 mmol) in benzotrifluoride (9 mL) was added pivalic acid (1.10 mL, 13.6 mmol), magnesium sulfate (540 mg, 4.50 mmol) and morpholine (91 mg, 0.90 mmol). The reaction was stirred at 175° C. under microwave irradiation for 2 h. The reaction was filtered through a pad of celite and washed with dichloromethane, and the filtrate was concentrated to remove solvent. The residue was purified by silica gel chromatography (0-10% methanol in dichloromethane) to give P15 (400 mg, 45%) as a brown solid. ¹H NMR (400 MHz, CDCl₃), δ: 11.00 (br. s, 1H), 6.17 (s, 1H), 3.84 (t, J=4.6 Hz, 4H), 3.01 (t, J=4.6 Hz, 4H), 2.31 (s, 3H).

### Preparation 16. 4-(6-chloro-3-methylpyridazin-4-yl)morpholine (P16)

A solution of 6-methyl-5-morpholinopyridazin-3(2H)-one (P15, 110 mg, 0.56 mmol) in POCl₃ (5 mL) was refluxed for overnight. The reaction was cooled and concentrated. The residue was diluted with ethyl acetate (100 mL) and washed with water (200 mL) and brine (200 mL). The organic layers were dried over NaSO₄₍ₛ₎, filtered, and concentrated to give a crude product (P16, 90 mg, 75% yield). ¹H NMR (400 MHz, CDCl₃), δ: 6.81 (s, 1H), 3.87 (t, J=4.8 Hz, 4H), 3.08 (t, J=4.8 Hz, 4H), 2.61 (s, 3H).

### Synthesis of 4-(6-(5-isopropoxy-1-trityl-1H-indazol-3-yl)-3-methylpyridazin-4-yl)morpholine (P17)

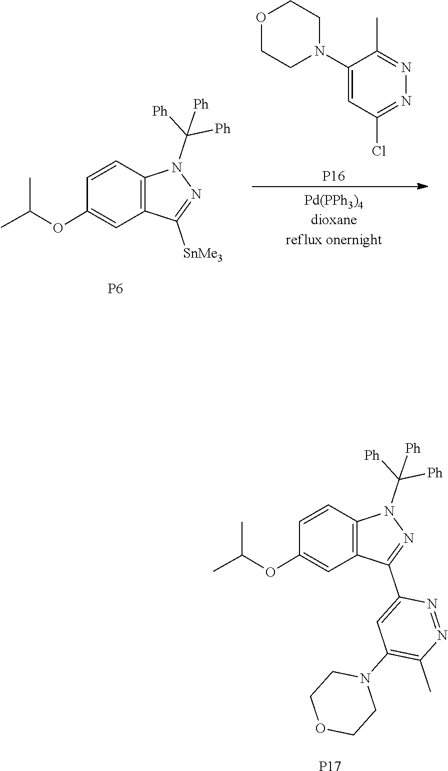

### Preparation 17. 4-(6-(5-isopropoxy-1-trityl-1H-indazol-3-yl)-3-methylpyridazin-4-yl)morpholine (P17)

To a solution of P6 (400 mg, crude) in dioxane (5.0 mL) at rt was added P16 (90 mg, 0.42 mmol) and Pd(PPh₃)₄ (50 mg, 0.04 mmol). The reaction was stirred at 100° C. for overnight under argon atmosphere. After it was cooled to room temperature, the mixture was filtered through a pad of celite, and the filtrate was concentrated. The residue was purified by preparative TLC (n-hexane:EtOAc=1:1) to give P17 (40 mg, 19% yield). ¹H NMR (400 MHz, CDCl₃), δ: 8.20 (d, J=2.4 Hz, 1H), 7.52 (s, 1H), 7.31-7.27 (m, 9H), 7.22-7.20 (m, 6H), 6.65 (dd, J=2.8, 9.4 Hz, 1H), 6.23 (d, J=9.4 Hz, 1H), 4.75-4.69 (m, 1H), 3.89-3.87 (m, 4H), 3.05-3.03 (m, 4H), 2.69 (s, 3H), 1.34 (d, J=6.0 Hz, 6H).

### Synthesis of 3-bromo-5-isopropoxy-1-((2-(trimethylsilyl)ethoxy)methyl)-1H-indazole (P19)

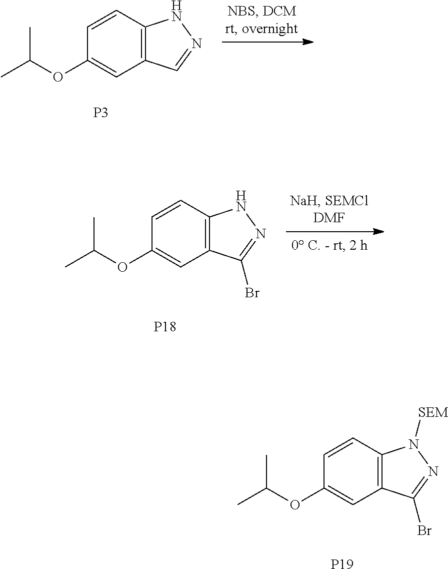

### Preparation 18. 3-bromo-5-isopropoxy-1H-indazole (P18)

To a solution of 5-isopropoxy-1H-indazole (P3, 3.0 g, 17 mmol) in DCM (30 mL) was added N-bromosuccinimide (3.32 g, 18.8 mmol) at 0° C. The mixture was stirred at rt for overnight. The reaction was directly concentrated and purified by silica gel column chromatography (n-hexane:EtOAc=1:1) to give P18 (4.11 g, 95% yield). ¹H NMR (400 MHz, CDCl₃), δ: 10.37 (br. s, 1H), 7.38 (d, J=9.0 Hz, 1H), 7.10 (dd, J=9.0, 2.4 Hz, 1H), 6.97 (d, J=2.4 Hz, 1H), 4.62 (septet, J=6.0 Hz, 1H), 1.38 (d, J=6.0 Hz, 6H).

### Preparation 19. 3-bromo-5-isopropoxy-1-((2-(trimethylsilyl)ethoxy)methyl)-1H-indazole (P19)

To a solution of P18 (4.11 g, 16.2 mmol) in DMF (40 mL) at 0° C. was added 60% NaH in mineral oil (780 mg, 19.4 mmol) under argon atmosphere. The reaction was stirred at 0° C. for 1 h. To the reaction was added 2-(trimethylsilyl)ethoxymethyl chloride (SEMCl, 3.42 mL, 19.4 mmol) at 0° C. After the mixture was stirred at rt for 2 h, the solution was poured into water and extracted with EtOAc. The organic phase was dried over MgSO₄₍ₛ₎, filtered, and concentrated. A residue was purified by silica gel column (n-hexane:DCM=2:1) to give P19 (4.75 g, 76% yield). ¹H NMR (400 MHz, CDCl₃), δ: 7.46 (d, J=9.0 Hz, 1H), 7.11 (dd, J=9.0, 2.4 Hz, 1H), 6.95 (d, J=2.4 Hz, 1H), 5.64 (s, 2H), 4.60 (septet, J=6.0 Hz, 1H), 3.57-3.53 (m, 1H), 1.38 (d, J=6.0 Hz, 6H), 0.89-0.85 (m, 2H), −0.06 (s, 9H).

### Synthesis of 4-(5-(5-isopropoxy-1-((2-(trimethylsilyl)ethoxy)methyl)-1H-indazol-3-yl)-1,2,4-triazin-3-yl)morpholine (P24)

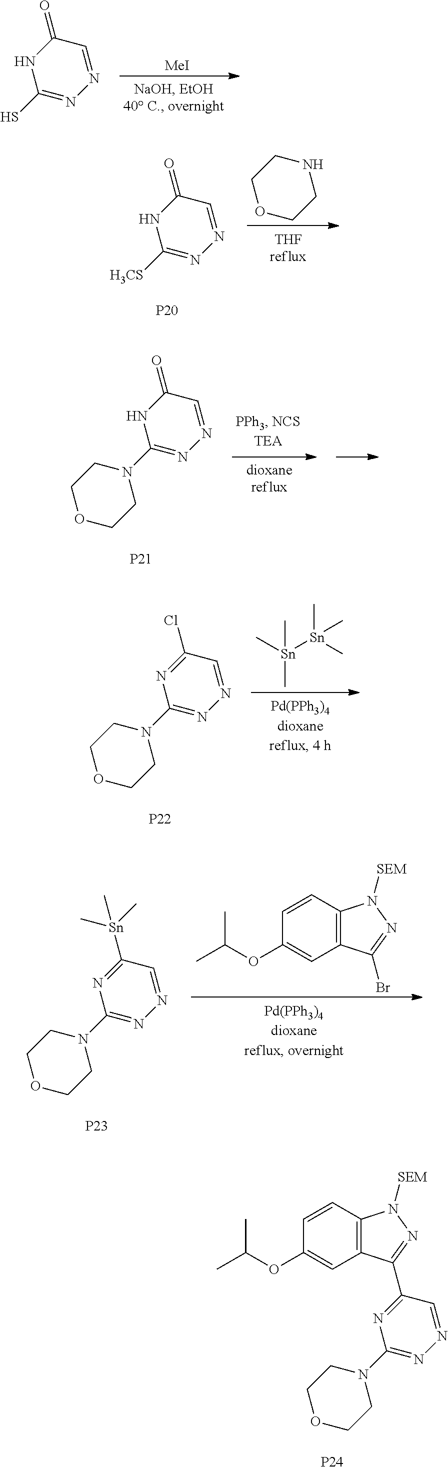

### Preparation 20. 3-(methylthio)-1,2,4-triazin-5(2H)-one (P20)

To a solution of 3-mercapto-1,2,4-triazin-5(4H)-one (1.66 g, 12.8 mmol) in ethanol (83 mL) at rt was added methyl iodide (1.60 mL, 25.6 mmol) and sodium hydroxide (620 mg, 15.5 mmol). The reaction was stirred at 40° C. for overnight. After it was cooled to rt, the mixture was dilute with water and extracted with EtOAc. The organic phase was dried over by MgSO₄₍ₛ₎, filtered, and concentrated. A residue was purified by silica gel column (methanol:DCM=20:1) to give P20 (556 mg, 31% yield). ¹H NMR (400 MHz, DMSO-d₆), δ: 7.58 (br. s, 1H), 2.46 (s, 3H).

### Preparation 21. 3-morpholino-1,2,4-triazin-5(4H)-one (P21)

To a solution of P20 (190 mg, 1.33 mmol) in THF (7.0 mL) at rt was added morpholine (340 mg, 3.98 mmol). The reaction was stirred at reflux for overnight. After it was cooled at 0° C. for 30 min, the resulting solid was collected by filtration and washed with EtOAc. The filtered cake was dried under high vacuum to give P21 (190 mg, 79% yield). ¹H NMR (400 MHz, DMSO-d₆), δ: 12.51 (br. s, 1H), 7.31 (s, 1H), 3.65-3.62 (m, 4H), 3.54-3.51 (m, 4H).

### Preparation 22. 4-(5-chloro-1,2,4-triazin-3-yl)morpholine (P22)

To a solution of triphenylphosphine (432 mg, 1.65 mmol) in dioxane (20 mL) was added N-chlorosuccinimide (220 mg, 1.65 mmol) at rt. After the mixture was stirred for 30 min at rt, P21 (100 mg, 0.55 mmol) was added, and the reaction mixture was stirred at reflux for 1 h. The reaction was cooled to 0° C. and triethylamine (110 mg, 1.10 mmol) was added. The mixture was stirred at 0° C. for another 1 h. The reaction mixture was concentrated and then purified by silica gel column chromatography (n-hexane:EtOAc=3:1) to give P22 (100 mg, 91% yield). ¹H NMR (400 MHz, DMSO-d₆), δ: 7.34 (s, 1H), 3.65-3.63 (m, 4H), 3.55-3.53 (m, 4H).

### Preparation 23. 4-(5-(trimethylstannyl)-1,2,4-triazin-3-yl)morpholine (P23)

To a solution of P22 (100 mg, 0.50 mmol) in dioxane (5.0 mL) at rt was added hexamethylditin (250 mg, 0.75 mmol) and tetrakis(triphenylphosphine)palladium(0) (60 mg, 0.05 mmol) under argon atmosphere. The reaction was stirred at 100° C. for overnight. After it was cooled to rt, the mixture was filtered through a pad of celite. The filtrate was concentrated to give P23 (100 mg, crude) which was used in next step without purification.

### Preparation 24. 4-(5-(5-isopropoxy-1-((2-(trimethylsilyl)ethoxy)methyl)-1H-indazol-3-yl)-1,2,4-triazin-3-yl)morpholine (P24)

To a solution of P23 (100 mg) in dioxane (5.0 mL) at rt was added P19 (211 mg, 0.55 mmol) and tetrakis(triphenylphosphine)palladium(0) (60 mg, 0.05 mmol) under argon atmosphere. The reaction was stirred at 100° C. for overnight. After it was cooled to rt, the mixture was filtered, and the filtrate was concentrated. The residue was purified by silica gel column chromatography (n-hexane:EtOAc=3:1) to give P24 (30 mg, 12% yield). ¹H NMR (400 MHz, CDCl₃), δ: 9.37 (s, 1H), 7.88 (d, J=2.2 Hz, 1H), 7.55 (d, J=9.0 Hz, 1H), 7.14 (dd, J=2.2, 9.0 Hz, 1H), 5.78 (s, 2H), 4.57 (septet, J=6.0 Hz, 1H), 4.05-4.02 (m, 4H), 3.89-3.87 (m, 4H), 3.61-3.57 (m, 2H), 1.41 (d, J=6.0 Hz, 6H), 0.93-0.89 (m, 2H), −0.06 (s, 9H).

### Synthesis of 6-(5-isopropoxy-1-trityl-1H-indazol-3-yl)-2-methyl-4-morpholinopyridazin-3(2H)-one (P29)

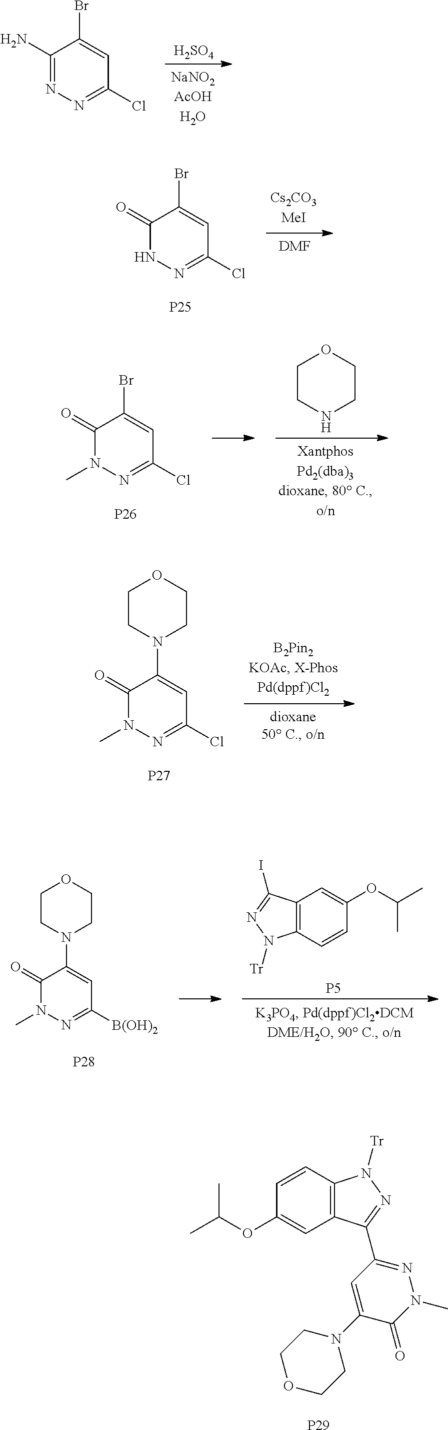

### Preparation 25. Methyl 4-bromo-6-chloropyridazin-3(2H)-one (P25)

To a solution of sodium nitrite (7.94 g, 115 mol) in conc. sulfuric acid (130 mL) was added a solution of 4-bromo-6-chloropyridazin-3-amine (20.00 g, 95.95 mol) in acetic acid (440 mL) in ice bath. The mixture was stirred at rt until the solution is clear, and then the mixture was added by water (660 mL). After the solution was stirred at rt for 2 days, the precipitate was collected by filtered and washed with water to give P25 (15.00 g, 75%) as a yellow solid which was used in next step without further purification. ¹H NMR (400 MHz, CDCl₃), δ: 7.68 (s, 1H).

### Preparation 26. 3-[6-(2,2,2-Trifluoroethyl)quinazolin-4-yl]-3,9-diazaspiro[5.5]undecane (P26)

To a solution of P25 (15.00 g, 71.62 mmol) and cesium carbonate (35.30 g, 108.0 mmol) in DMF (115 mL) was added iodomethane (6.70 mL, 108 mmol) in ice bath. After the reaction was stirred at rt for overnight, the mixture was treated with water and extracted with EtOAc. The combined organic layers were dried over Na₂SO₄, filtered, and concentrated. The residue was purified by silica gel chromatography (0-30% EtOAc in n-hexane) to give P26 (9.79 g, 61%) as a yellow solid. ¹H NMR (400 MHz, CDCl₃), δ: 7.62 (s, 1H), 3.80 (s, 3H).

### Preparation 27. 6-Chloro-2-methyl-4-morpholinopyridazin-3(2H)-one (P27)

To a solution of P26 (1.5 g, 6.71 mmol) in 1,4-dioxane (56 mL) was added morpholine (0.60 mL, 6.7 mmol), cesium carbonate (4.37 g, 13.4 mmol), and Xantphos (388 mg, 0.67 mmol). The mixture was bubbled with argon for 15 min. Tris(dibenzylideneacetone)dipalladium(0) (307 mg, 0.34 mmol) was added to the mixture and the solution was stirred at 80° C. for overnight. The mixture was filtered through a pad of celite. The filtrate was treated with water and extracted with EtOAc. The combined organic layers were dried over Na₂SO₄, filtered, and concentrated. The residue was purified by silica gel chromatography (0-20% EtOAc in n-hexane) to give P27 (1.23 g, 80%) as an orange oil. ¹H NMR (400 MHz, CDCl₃), δ: 6.23 (s, 1H), 3.84 (dd, J=5.7, 4.0 Hz, 4H), 3.70 (s, 3H), 3.52 (dd, J=5.7, 4.0 Hz, 4H).

### Preparation 28. (1-Methyl-5-morpholino-6-oxo-1,6-dihydropyridazin-3-yl)boronic acid (P28)

To a solution of P27 (1.20 g, 5.23 mmol) in 1,4-dioxane (60 mL) was added bis(pinacolato)diboron (6.63 g, 26.12 mmol) and potassium acetate (1.026 g, 10.45 mmol). The mixture was bubbled with argon for 15 min. XPhos (500 mg, 1.05 mmol) and [1,1′-Bis(diphenylphosphino)ferrocene]dichloropalladium(II) (382 mg, 0.52 mmol) was added to the mixture. The solution was bubbled with argon for 15 min. After the mixture was stirred at 50° C. for 16 h, the solution was filtered through a pad of celite, and the filtrate was concentrated to give P28 as a crude product which was used in next step without further purification.

### Preparation 29. 6-(5-Isopropoxy-1-trityl-1H-indazol-3-yl)-2-methyl-4-morpholinopyridazin-3(2H)-one (P29)

To a solution of P28 (5.23 mmol) in DME (50 mL) was added P5 (2.37 g, 4.35 mmol), potassium phosphate (2M solution in water, 2.77 g, 13.1 mmol). The mixture was bubbled with argon for 15 min. [1,1′-Bis(diphenylphosphino)ferrocene]-dichloropalladium (II) dichloromethane complex (356 mg, 0.44 mmole) was added to the solution. After the reaction was stirred at 90° C. for overnight, the mixture was filtered through a pad of celite. The filtrate was concentrated in vacuo and then purified by silica gel chromatography (0-30% EtOAc in n-hexane) to give 4.6 g of P29. ¹H NMR (400 MHz, CDCl₃), δ: 7.79 (d, J=2.4 Hz, 1H), 7.29-7.26 (m, 8H), 7.20-7.17 (m, 7H), 7.06 (s, 1H), 6.65 (dd, J=9.2, 2.4 Hz, 1H)), 6.21 (d, J=9.2 Hz, 1H), 4.59 (septet, J=6.0 Hz, 1H), 3.88 (s, 3H), 3.88-3.84 (m, 4H), 3.43-3.40 (m, 4H), 1.37 (d, J=6.0 Hz, 6H).

### Synthesis of 3-Iodo-4-isopropoxy-1-trityl-1H-indazole (P32)

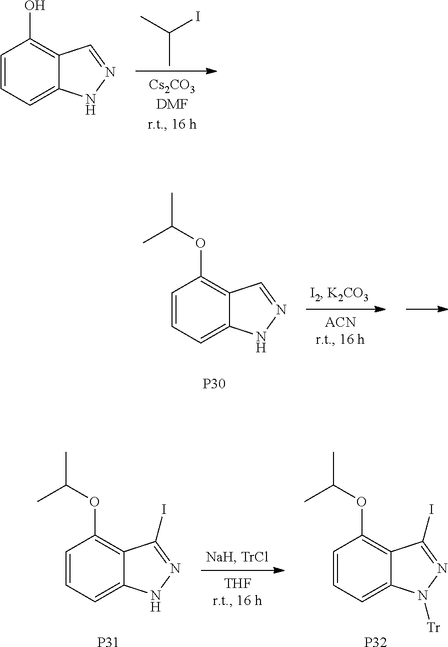

### Preparation 30. 4-Isopropoxy-1H-indazole (P30)

To a solution of 1H-indazol-4-ol (3.0 g, 22.3 mmol, 1.0 eq) in DMF (29 mL) was added 2-iodopropane (2.60 mL, 26.8 mmol, 1.2 eq) and cesium carbonate (8.71 g, 26.8 mmol, 1.2 eq). The reaction was stirred at rt for 16 h. The reaction was treated with water and extracted with ethyl acetate for three times. The combined organic layers were dried over Na₂SO₄₍ₛ₎, filtered, and concentrated in vacuo to give the residue, which was purified by silica gel column chromatography (0-100% ethyl acetate in n-hexane) to give P30 (3.2 g, 87% yield) as a yellow solid. ¹H NMR (400 MHz, CDCl₃), δ: 9.99 (br. s, 1H), 8.13 (d, J=1.2 Hz, 1H), 7.29-7.25 (m, 1H), 7.04-7.01 (m, 1H), 6.50-6.48 (m, 1H), 4.75 (septet, J=6.0 Hz, 1H), 1.43 (d, J=6.0 Hz, 6H).

### Preparation 31. 3-Iodo-4-isopropoxy-1H-indazole (P31)

To a solution of P30 (3.2 g, 18.0 mmol, 1.0 eq) in acetonitrile (90 mL) was added potassium carbonate (4.9 g, 36.0 mmol, 2.0 eq) and iodine (6.8 g, 27.0 mmol, 1.5 eq). The reaction was stirred at rt for 16 h. The mixture was quenched with Na₂S₂O_{3(aq)} and extracted with ethyl acetate for three times. The combined organic layers were dried over Na₂SO₄₍ₛ₎, filtered, and concentrated in vacuo to give the residue, which was purified by silica gel column chromatography (0-50% ethyl acetate in n-hexane) to give P31 (2.0 g, 37% yield) as a yellow oil. ¹H NMR (400 MHz, CDCl₃), δ: 10.28 (br. s, 1H), 7.29-7.25 (m, 1H), 7.02 (d, J=8.4 Hz, 1H), 6.48 (d, J=7.6 Hz, 1H), 4.73 (septet, J=6.0 Hz, 1H)), 1.47 (d, J=6.0 Hz, 6H).

### Preparation 32. 3-Iodo-4-isopropoxy-1-trityl-1H-indazole (P32)

To a solution of P31 (2.0 g, 6.6 mmol, 1.0 eq) in THF (33 mL) was added sodium hydride (190 mg, 7.9 mmol, 1.2 eq) and TrCl (2.0 g, 7.2 mmol, 1.1 eq) at 0° C. The reaction was stirred at rt for 16 h. The mixture was quenched with water and extracted with ether for three times. The combined organic layers were dried over Na₂SO₄₍ₛ₎, filtered, and concentrated in vacuo to give the residue, which was purified by silica gel column chromatography (0-50% ethyl acetate in n-hexane) to give P32 (3.3 g, 92% yield) as a yellow oil. ¹H NMR (400 MHz, CDCl₃), δ: 7.25-7.23 (m, 8H), 7.21-7.17 (m, 7H), 6.84-6.80 (m, 1H), 6.34 (d, J=7.6 Hz, 1H), 6.93 (d, J=8.4 Hz, 1H), 4.67 (septet, J=6.0 Hz, 1H), 1.45 (d, J=6.0 Hz, 6H).

### Synthesis of 6-(4-Isopropoxy-1-trityl-1H-indazol-3-yl)-2-methyl-4-morpholinopyridazin-3(2H)-one (P33)

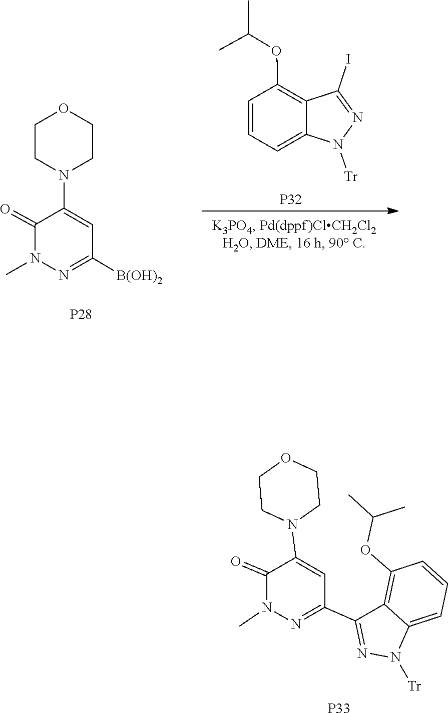

### Preparation 33. 6-(4-Isopropoxy-1-trityl-1H-indazol-3-yl)-2-methyl-4-morpholinopyridazin-3(2H)-one (P33)

To a solution of (1-methyl-5-morpholino-6-oxo-1,6-dihydropyridazin-3-yl)boronic acid (P28, 2.20 mmol) in DME (20 mL) was added 3-iodo-4-isopropoxy-1-trityl-1H-indazole (P32, 1.00 g, 1.84 mmol) and potassium phosphate (2M in H₂O, 2.75 mL, 5.51 mmol). The mixture was bubbled with argon for 15 min. [1,1′-Bis(diphenylphosphino)ferrocene]dichloropalladium(II) dichloromethane complex (150 mg, 0.184 mmol) was added to the mixture, and it was degassed with argon. After the mixture was stirred at 90° C. for overnight, the solution was treated with EtOAc and filtered through a pad of celite. The filtrate was concentrated in vacuo and then purified by silica gel chromatography (0-50% EtOAc in n-hexane) to give P33 (812 mg, 72%) as a brown solid. ¹H NMR (400 MHz, CDCl₃), δ: 7.26-7.19 (m, 15H), 6.88 (dd, J=8.4, 7.8 Hz, 1H), 6.79 (s, 1H), 6.42 (d, J=7.8 Hz, 1H), 6.02 (d, J=8.4 Hz, 1H), 4.766 (septet, J=6.0 Hz, 1H), 3.89-3.82 (m, 7H), 3.47-3.39 (m, 4H), 1.34 (d, J=6.0 Hz, 6H).

### Synthesis of 4-(2,7-Diazaspiro[3.5]non-2-yl)-6-(2,2,2-trifluoroethyl)cinnoline (P35)

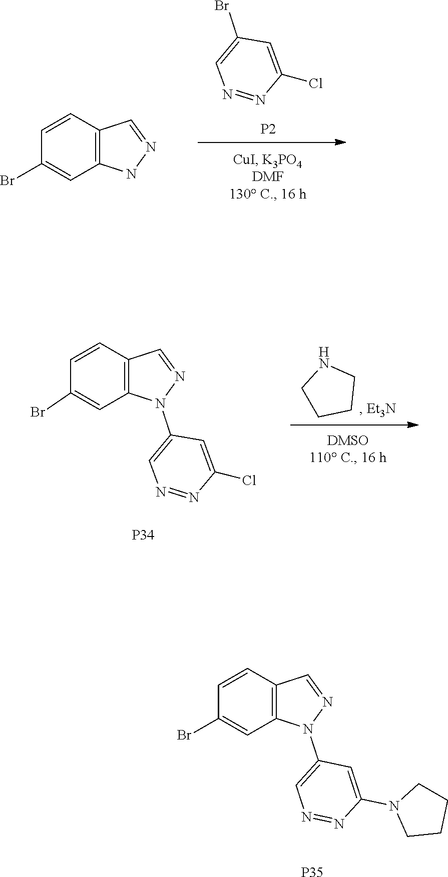

### Preparation 34. 6-Bromo-1-(6-chloropyridazin-4-yl)-1H-indazole (P34)

To a solution of 6-bromo-1H-indazole (100.0 mg, 0.508 mmol, 1.0 eq) in DMF (1.0 mL) was added P2 (147.3 mg, 0.761 mmol, 1.5 eq), copper(I) iodide (19.3 mg, 0.102 mmol, 0.2 eq) and tripotassium phosphate (215.5 mg, 1.02 mmol, 2.0 eq). The reaction mixture was stirred at rt for 5 min and then at 130° C. for 16 h. After the solution was cooled to rt, it was treated with water and extracted with DCM for three times. The combined organic layers were dried over Na₂SO₄₍ₛ₎, filtered, and concentrated in vacuo to give the residue, which was purified by silica gel column chromatography (n-hexane:EtOAc=6:1 to 5:1) to give P34 (62 mg, 39% yield) as a light-yellow solid. ¹H NMR (400 MHz, CDCl₃), δ: 9.78 (d, J=2.4 Hz, 1H), 8.31 (d, J=0.8 Hz, 1H), 8.10 (d, J=1.6 Hz, 1H), 7.92 (d, J=2.4 Hz, 1H), 7.74 (dd, J=8.4, 0.8 Hz, 1H), 7.52 (dd, J=8.4, 1.6 Hz, 1H).

### Preparation 35. 6-Bromo-1-(6-(pyrrolidin-1-yl)pyridazin-4-yl)-1H-indazole (P35)

To a solution of P34 (62.0 mg, 0.20 mmol, 1.0 eq) in DMSO (1.0 mL) was added triethylamine (0.17 mL, 1.20 mmol, 6.0 eq) and pyrrolidine (0.05 mL, 0.60 mmol, 3.0 eq). After the solution was stirred at 110° C. for 16 h and cooled to rt, it was treated with water and extracted with DCM for three times. The combined organic layers were dried over Na₂SO₄₍ₛ₎, filtered, and concentrated in vacuo to give the residue, which was purified by silica gel chromatography (DCM:MeOH=50:1) to give P35 (58 mg, 84% yield) as a light-yellow solid. ¹H NMR (400 MHz, CDCl₃), δ: 9.08 (d, J=2.2 Hz, 1H), 8.23 (s, 1H), 8.08 (s, 1H), 7.69 (d, J=8.4 Hz, 1H), 7.42 (d, J=8.4 Hz, 1H), 6.92 (d, J=2.2 Hz, 1H), 3.68-3.61 (m, 4H), 2.14-2.07 (m, 4H).

### Synthesis of 2-(1-(6-Chloropyridazin-4-yl)-1H-indazol-6-yl)-2-methylpropanenitrile (P36)

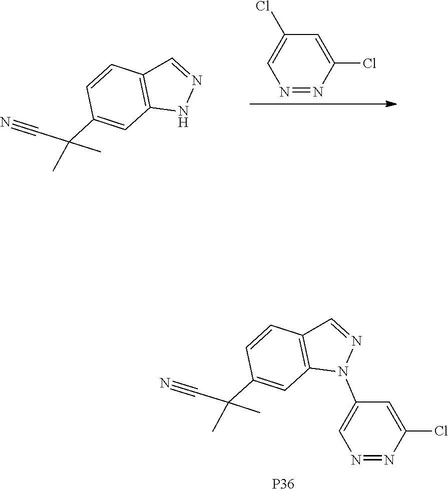

### Preparation 36. 2-(1-(6-Chloropyridazin-4-yl)-1H-indazol-6-yl)-2-methylpropanenitrile (P36)

40 mg of NaH (60% in mineral oil, 1 mmol) was added in one portion to the stirred solution of 2-(1H-indazol-6-yl)-2-methyl-propanenitrile (185 mg, 1 mmol) and 3,5-dichloropyridazine (148 mg, 1 mmol) in 2 mL of DMF at rt. The reaction mixture was stirred at rt for 8 h before been poured onto cold water (50 mL). Precipitate was filtered off and dried under reduced pressure. Yield of P36 150 mg (53%). LCMS (C18 column 20×2 mm, 2.5 μm, pore size 100 Å, water-acetonitrile+0.1% TFA, gradient 5 to 87% for 3 min, retention time 1.52 min). MS (ESI) m/z: 298.5 [M+H]+. ¹H NMR (400 MHz, DMSO-d₆), δ: 9.91 (d, J=2.2 Hz, 1H), 8.66 (s, 1H), 8.33 (d, J=2.2 Hz, 1H), 8.01 (d, J=8.5 Hz, 1H), 7.55 (dd, J=8.5, 1.5 Hz, 1H), 7.14 (d, J=2.2 Hz, 1H), 1.80 (s, 6H).

### Synthesis of 1-(1-(6-Chloropyridazin-4-yl)-1H-indazol-6-yl)cyclohexanecarbonitrile

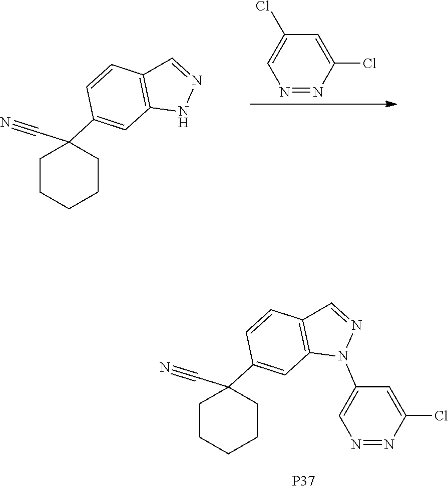

### Preparation 37. 1-[1-(6-chloropyridazin-4-yl)indazol-6-yl]cyclohexanecarbonitrile (P37)

40 mg of NaH (60% in mineral oil, 1 mmol) was added in one portion to the stirred solution of 1-(1H-indazol-6-yl)cyclohexanecarbonitrile (225 mg, 1 mmol) and 3,5-dichloropyridazine (148 mg, 1 mmol) in 2 mL of DMF at rt. The reaction mixture was stirred at rt for 8 h before been poured into cold water (50 mL). Formed precipitate was filtered off and dried under reduced pressure. Yield of P37 240 mg (71%). LCMS (C18 column 20×2 mm, 2.5 μm, pore size 100 Å, water-acetonitrile+0.1% TFA, gradient 5 to 87% for 3 min, retention time 1.76 min). MS (ESI) m/z: 338.5 [M+H]+. ¹H NMR (400 MHz, DMSO-d₆), δ: 9.91 (d, J=2.2 Hz, 1H), 8.66 (s, 1H), 8.33 (d, J=2.2 Hz, 1H), 8.17 (s, 1H), 8.01 (d, J=8.5 Hz, 1H), 7.64 (d, J=8.5 Hz, 1H), 2.21 (d, J=12.9 Hz, 2H), 2.07-2.00 (m, 2H), 1.91-1.88 (d, 2H), 1.80-1.64 (m, 3H), 1.42-1.32 (m, 1H).

### Synthesis of 1-[1-(6-Chloropyridazin-4-yl)indazol-6-yl]cyclopentanecarbonitrile (P38)

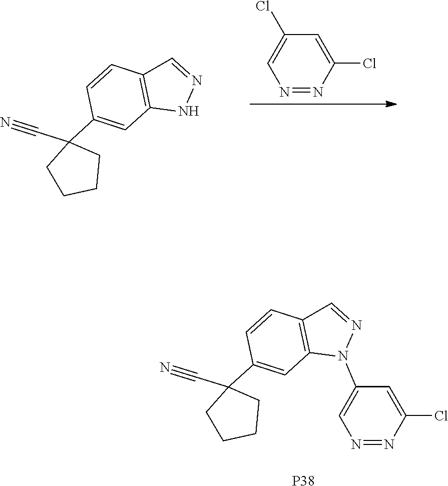

### Preparation 38. 1-[1-(6-Chloropyridazin-4-yl)indazol-6-yl]cyclopentanecarbonitrile (P38)

40 mg of NaH (60% in mineral oil, 1 mmol) was added in one portion to the stirred solution of 1-(1H-indazol-6-yl)cyclopentanecarbonitrile (211 mg, 1 mmol) and 3,5-dichloropyridazine (148 mg, 1 mmol) in 2 mL of DMF at rt. The reaction mixture was stirred at rt for 8 h before been poured onto cold water (50 mL). Formed precipitate was filtered off and dried under reduced pressure. Yield of P38 was 241 mg (74%). LCMS (C18 column 20×2 mm, 2.5 μm, pore size 100 Å, water-acetonitrile+0.1% TFA, gradient 5 to 87% for 3 min, retention time 1.65 min). MS (ESI) m/z: 324.5 [M+H]⁺. ¹H NMR (400 MHz, DMSO-d₆), δ: 9.90 (d, J=2.2 Hz, 1H), 8.67 (s, 1H), 8.35 (d, J=2.2 Hz, 1H), 8.17 (s, 1H), 8.01 (d, J=8.5 Hz, 1H), 7.58 (d, J=8.5 Hz, 1H), 2.60-2.55 (m, 2H), 2.29-2.21 (m, 2H), 1.98-1.95 (m, 4H).

### Synthesis of 1-[1-(6-chloropyridazin-4-yl)-1H-indazol-6-yl]-1H-pyrrole-2-carbonitrile (P42)

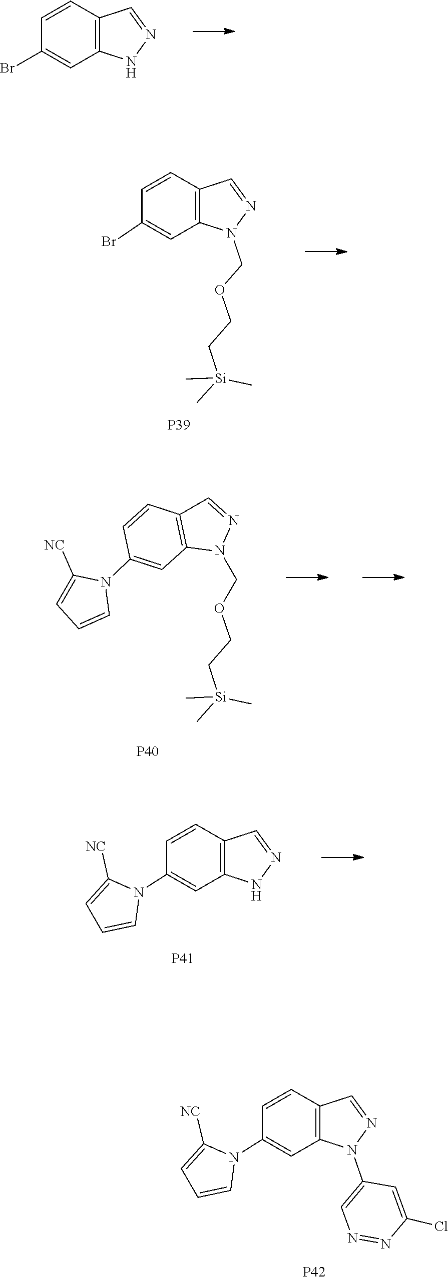

### Preparation 39. 6-bromo-1-{[2-(trimethylsilyl)ethoxy]methyl}-1H-indazole (P39)

To an ice-cold solution of 6-bromo-1H-indazole (10 g, 50.7 mmol) in DMF (50 ml) 60% dispersion of NaH in mineral oil (2.2 g, 55 mmol) at 0° C., and the reaction was stirred for an hour. Then SEM-Cl (10 ml, 9.42 g, 56.5 mmol) was added to the mixture. The resulting mixture was stirred at 0° C. for 1 hour and overnight at ambient temperature. Then solvent was evaporated in vacuo and the residue was partitioned between DCM and water (100 ml each). Organic layer was separated; aqueous was extracted with DCM (50 ml). Combined organic layers were dried over MgSO₄, filtered and concentrated. The residue was purified via silica gel chromatography (gradient elution: 50-0% hexane/CHCl₃) to provide 8.2 g (49%) of compound P39. ¹H NMR (400 MHz, CDCl₃), δ: 7.99 (s, 1H), 7.79 (s, 1H), 7.61 (d, J=8.5 Hz, 1H), 7.32 (dd, J=8.5, 1.3 Hz, 1H), 5.71 (s, 2H), 3.57 (dd, J=25.9, 17.8 Hz, 2H), 1.00-0.80 (m, 2H), −0.05 (s, 9H).

### Preparation 40. 1-(1-{[2-(Trimethylsilyl)ethoxy]methyl}-1H-indazol-6-yl)-1H-pyrrole-2-carbonitrile (P40)

Compound P39 (1 g, 3.05 mmol), copper iodide (60 mg, 0.31 mmol), sodium iodide (0.96 g, 6.4 mmol), 2-cyanopyrrole (0.3 g, 3.2 mmol) and K₃PO₄ (1.4 g, 6.6 mmol) are weighed out into a vial, a stir bar was added; the vial was sealed and was purged with nitrogen. 1,4-Dioxane (3 mL) was added, followed by trans-N,N′-dimethyl-1,2-diamine (0.04 mg, 0.032 g, 0.37 mmol). The mixture was heated to 110° C. for overnight. The mixture was evaporated in vacuo and was then diluted with DCM and water. The mixture was filtered through a hydrophobic frit and was then purified via column chromatography to afford compound P40 (0.73 g, 67%). LCMS (C18 column 20×2 mm, 2.5 μm, pore size 100 Å, water-acetonitrile+0.1% TFA, gradient 5 to 87% for 3 min, retention time 2.02 min). MS (ESI) m/z: 339.5 [M+H]⁺. ¹H NMR (400 MHz, CDCl₃), δ: 8.10 (s, 1H), 7.88 (d, J=8.5 Hz, 1H), 7.67 (s, 1H), 7.33 (dd, J=8.5, 1.6 Hz, 1H), 7.18 (d, J=1.8 Hz, 1H), 7.05 (dd, J=3.9, 1.4 Hz, 1H), 6.45-6.36 (m, 1H), 5.79 (s, 2H), 3.71-3.46 (m, 2H), 1.02-0.81 (m, 2H), −0.01-−0.14 (m, 9H).

### Preparation 41. 1-(1H-Indazol-6-yl)-1H-pyrrole-2-carbonitrile (P41)

Compound P40 (0.4 g, 1.18 mmol) was dissolved in DCM (10 ml) and TFA (3.4 ml, 5.05 g, 44.2 mmol). The mixture was stirred at ambient temperature for 48 h. The mixture was then evaporated in vacuo. The residue was treated with aqueous NaHCO₃, and the product was extracted with ethyl acetate (2 times by 10 ml). The combined organic layers were dried over MgSO₄ and concentrated. The product P41 (0.2 g, 81%) was used further without additional purification. LCMS (C18 column 20×2 mm, 2.5 μm, pore size 100 Å, water-acetonitrile+0.1% TFA, gradient 5 to 87% for 3 min, retention time 1.47 min). MS (ESI) m/z 209.4 [M+H]⁺. ¹H NMR (400 MHz, CDCl₃), δ: 10.80 (br. s, 1H), 8.18 (s, 1H), 7.89 (d, J=8.5 Hz, 1H), 7.64 (s, 1H), 7.32-7.23 (m, 1H), 7.18 (dd, J=2.3, 1.3 Hz, 1H), 7.09-7.02 (m, 1H), 6.46-6.37 (m, 1H).

### Preparation 42. 1-[1-(6-Chloropyridazin-4-yl)-1H-indazol-6-yl]-1H-pyrrole-2-carbonitrile (P42)

To a solution of compound P41 (240 mg, 1.15 mmol) in anhydrous DMF (5 mL) was added 60% dispersion of NaH in mineral oil (70.0 mg, 1.75 mmol) at 0° C., and the reaction was stirred for 20 min. Then 3,5-dichloropyridazine (0.2 mg, 1.34 mmol) was added to the mixture. The resulting mixture was stirred at 0° C. for 1 h. The reaction mixture was poured into water (10 mL) and extracted with EtOAc (3×30 mL). The organic layer was washed with water (10 mL), dried over MgSO₄ filtered and concentrated. The residue was purified via silica gel chromatography (gradient elution: 0-22% EtOAc/Hexanes) to provide 0.17 g (45%) of compound P42. LCMS (C18 column 20×2 mm, 2.5 μm, pore size 100 Å, water-acetonitrile+0.1% TFA, gradient 5 to 87% for 3 min, retention time 1.55 min). MS (ESI) m/z: 321.4 [M+H]⁺.

### Synthesis of 1-(1H-indazol-6-yl)cyclopropanecarbonitrile (P43)

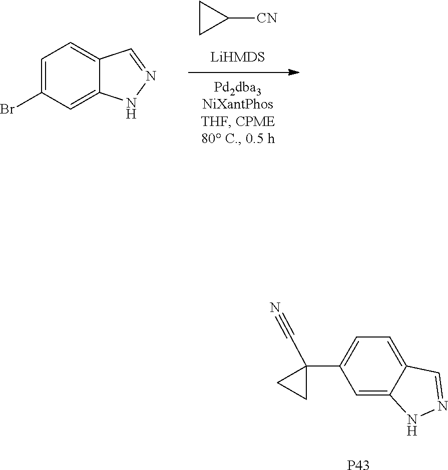 [SMILES: N#CC1CC1 ; Brc1ccc2cn[nH]c2c1 ; "P43" ; N#CC1(c2ccc3cn[nH]c3c2)CC1]

### Preparation 43. 1-(1H-indazol-6-yl)cyclopropanecarbonitrile (P43)

To a solution of 6-bromo-1H-indazole (5.0 g, 25 mmol), cyclopropanecarbonitrile (2.6 g, 39 mmol), Pd₂dba₃ (1.2 g, 1.3 mmol) and NiXantPhos (1.4 g, 2.5 mmol) in THF/CPME (1:1 (v/v), 150 mL) was added LiHMDS (76 mL, 76 mmol, 1 M in THF) at 0° C. After the reaction was stirred at 80° C. for 30 min, the mixture was treated with NH₄Cl_{(aq)} and extracted with EtOAc. The organic layers were dried over Na₂SO₄₍ₛ₎, filtered, and concentrated under reduced pressure. The residue was purified by silica gel column chromatography (25% EtOAc in n-hexane) to afford P43 (2.95 g, 64%) as a yellow solid. ¹H NMR (400 MHz, CDCl₃): δ 10.51 (br. s, 1H), 8.07 (s, 1H), 7.74 (dd, J=8.6 Hz, 1H), 7.57 (s, 1H), 7.00 (d, J=8.6 Hz, 1H), 1.81-1.78 (m, 2H), 1.51-1.47 (m, 2H); LRMS (ESI) m/z: calc. for C₁₁H₉N₃ 183.08; found, 184.1 [M+H]⁺; HPLC purity: 99.27%, t_{R}=14.56 min.

### Synthesis of 2-(1H-indazol-6-yl)-2-methylpropanenitrile (P44)

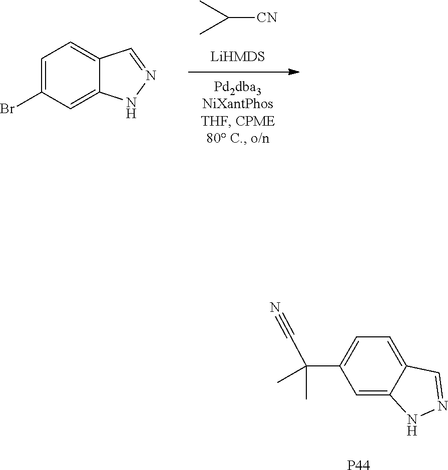

### Preparation 44. 2-(1H-indazol-6-yl)-2-methylpropanenitrile (P44)

To a solution of 6-bromo-1H-indazole (2.00 g, 10.2 mmol), isobutyronitrile (1.06 g, 15.3 mmol), Pd₂dba₃ (468 mg, 0.510 mmol) and NiXantPhos (564 mg, 1.02 mmol) in THF/CPME (1:1 (v/v), 60 mL) was added LiHMDS (30 mL, 30.45 mmol, 1 M in THF) at 0° C. After the reaction was stirred at 80° C. for overnight, the mixture was treated with NH₄Cl_{(aq)} and extracted with EtOAc. The organic layers were dried over Na₂SO₄₍ₛ₎, filtered, and concentrated under reduced pressure. The residue was purified by silica gel column chromatography (25% EtOAc in n-hexane) to afford P44 (1.16 g, 62%) as a yellow oil. ¹H NMR (400 MHz, CDCl₃), δ: 10.34 (br. s, 1H), 8.08 (s, 1H), 7.79 (d, J=8.4 Hz, 1H), 7.68 (s, 1H), 7.26 (dd, J=8.4 Hz, 1.6 Hz, 1H), 1.80 (s, 6H); LRMS (ESI) m/z: calc. for C₁₁H₁₁N₃ 185.10; found, 186.2 [M+H]⁺; HPLC purity: 94.14%, t_{R}=15.43 min.

### Synthesis of 2-cyclopropyl-6-(5-isopropoxy-1-trityl-1H-indazol-3-yl)-4-morpholinopyridazin-3(2H)-one (P48)

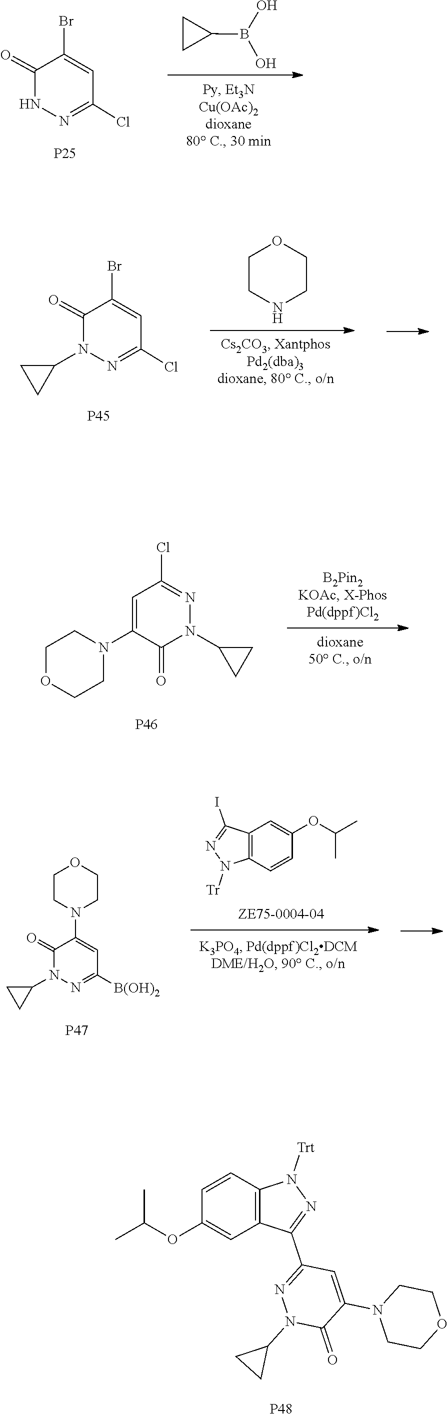

### Preparation 45. 4-Bromo-6-chloro-2-cyclopropylpyridazin-3(2H)-one (P45)

To a solution of P25 (1.50 g, 7.16 mmol) in 1,4-dioxane (10 mL) was added cyclopropyl boronic acid (1.85 g, 21.5 mmol), pyridine (4.57 mL, 57.3 mmol) and triethylamine (5.00 mL, 35.8 mmol). The mixture was bubbled with argon for 15 min. Copper(II) acetate (1.30 g, 7.16 mmol) was added to the mixture, and it was degassed with argon. After the mixture was stirred at 80° C. for 30 min, the solution was treated with water and extracted with EtOAc. The combined organic layers were dried over MgSO₄, filtered, and concentrated. The residue was purified by silica gel chromatography (0-15% EtOAc in n-hexane) to give P45 (252 mg, 14%) as a white solid. ¹H NMR (400 MHz, CDCl₃), δ: 7.57 (s, 1H), 4.11 (s, 1H), 1.13-1.12 (m, 2H), 1.05-1.04 (m, 2H); LCMS (ESI) m/z calc. for C₇H₆BrClN₂O 247.94; found, 248.6 [M+H]⁺.

### Preparation 46. 6-chloro-2-cyclopropyl-4-morpholinopyridazin-3(2H)-one (P46)

To a solution of P45 (250 mg, 1.00 mmol) in 1,4-dioxane (10 mL) was added morpholine (0.087 mL, 1.00 mmol) and cesium carbonate (652 mg, 2.00 mmol). The mixture was bubbled with argon for 15 min. Xantphos (57.9 mg, 0.100 mmol) and tris(dibenzylideneacetone)dipalladium(0) (45.8 mg, 0.050 mmol) was added to the mixture, and it was degassed with argon. After the mixture was stirred at 80° C. for overnight, the solution was filtered through a pad of celite and the filtrate was treated with water and extracted with EtOAc. The combined organic layers were dried over MgSO₄, filtered, and concentrated. The residue was purified by silica gel chromatography (0-40% EtOAc in n-hexane) to give P46 (183 mg, 71%) as a yellow gum. ¹H NMR (400 MHz, CDCl₃), δ: 6.20 (s, 1H), 3.94-3.87 (m, 1H), 3.87-3.81 (m, 4H), 3.55-3.48 (m, 4H), 1.13-0.94 (m, 4H); LCMS (ESI) m/z calc. for C₁₁H₁₄C₁N₃O₂ 255.08; found, 255.9 [M+H]⁺.

### Preparation 47. (1-Cyclopropyl-5-morpholino-6-oxo-1,6-dihydropyridazin-3-yl)boronic acid (P47)

To a solution of P46 (180 mg, 0.704 mmol) in 1,4-dioxane (7.0 mL) was added bis(pinacolato)diboron (894 mg, 3.52 mmol) and potassium acetate (138 mg, 1.41 mmol). The mixture was bubbled with argon for 15 min. XPhos (67 mg, 0.14 mmol) and [1,1′-Bis(diphenylphosphino)ferrocene]dichloropalladium(II) (52 mg, 0.070 mmol) was added to the mixture and the solution was degassed with argon for 15 min. After the mixture was stirred at 50° C. for 16 h, the solution was filtered through a pad of celite and the filtrate was concentrated to give P47 as a crude product which was used in next step without further purification.

### Preparation 48. 2-Cyclopropyl-6-(5-isopropoxy-1-trityl-1H-indazol-3-yl)-4-morpholinopyridazin-3(2H)-one (P48)

To a solution of P47 (crude, 0.704 mmol) in 1,2-dimethoxyethane (7.0 mL) was added P5 (319 mg, 0.586 mmol) and potassium phosphate (2M in H₂O, 0.80 mL, 1.8 mmol). The mixture was degassed with argon for 15 min. [1,1′-Bis(diphenylphosphino)ferrocene]dichloropalladium(II) complex with DCM (48 mg, 0.059 mmol) was added to the mixture, and it was degassed with argon again. After the mixture was stirred at 90° C. for overnight, the solution was diluted with EtOAc and filtered through a pad of celite. The filtrate was concentrated in vacuo and then purified by silica gel chromatography (0-40% EtOAc in n-hexane) to give P48 (316 mg, 69%) as a brown solid. ¹H NMR (400 MHz, CDCl₃), δ: 7.72 (d, J=2.4 Hz, 1H), 7.31-7.27 (m, 8H), 7.20-7.14 (m, 7H), 7.04 (s, 1H), 6.63 (dd, J=9.2, 2.4 Hz, 1H), 6.21 (d, J=9.2 Hz, 1H), 4.63-4.54 (m, 1H), 4.32-4.22 (m, 1H), 3.87-3.82 (m, 4H), 3.47-3.38 (m, 4H), 1.41 (s, 3H), 1.40 (s, 3H), 1.10-1.03 (m, 4H); LCMS (ESI) m/z calc. for C₄₀H₃₉N₅O₃ 637.31; found, 638.5 [M+H]⁺.

### Synthesis of 2-methyl-6-(5-(1-methylcyclopropoxy)-1-(tetrahydro-2H-pyran-2-yl)-1H-indazol-3-yl)-4-morpholinopyridazin-3(2H)-one (P51)

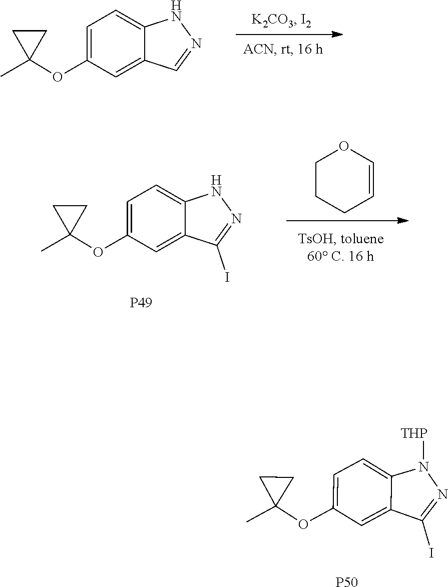

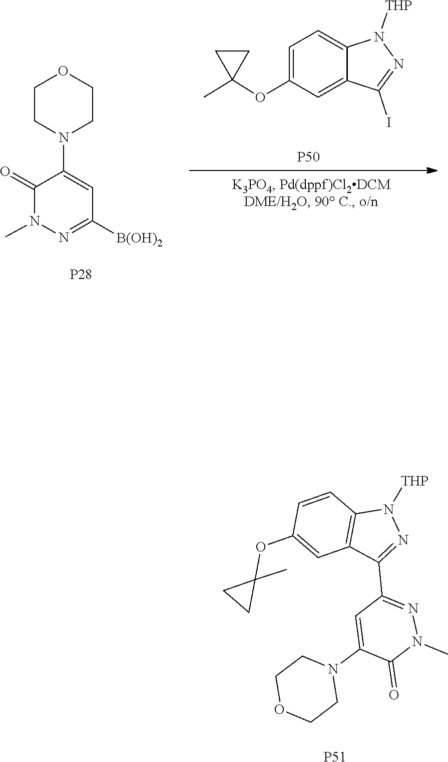

### Preparation 49. 5-(1-Methylcyclopropoxy)-2-((2-(trimethylsilyl)ethoxy)methyl)-2H-indazole (P49)

To a solution of 5-(1-methylcyclopropoxy)-1H-indazole (1.9 g, 10.09 mmol) in acetonitrile (50 mL) was added iodine (3.5 g, 14 mmol), was added potassium carbonate (1.5 g, 27 mmol) and iodine (3.84 g, 15.14 mmol) at rt. The mixture was stirred at rt for 16 h. The mixture was concentrated to remove solvent. The residue was purified by silica gel column chromatography (0-50% ethyl ethanoate in n-hexane) to give P49 (1.8 g, 57%) as a yellow oil. ¹H NMR (400 MHz, CDCl₃), δ: 7.37 (d, J=8.8 Hz, 1H), 7.12-7.09 (m, 1H), 7.05 (d, J=2.4 Hz, 1H), 1.07-1.04 (m, 2H), 0.79-0.76 (m, 2H).

### Preparation 50. 3-(6-Chloropyrimidin-4-yl)-5-(1-methylcyclopropoxy)-2-((2-(trimethylsilyl)ethoxy)methyl)-2H-indazole (P50)

A solution of P49 (2.2 g, 7.0 mmol), p-toluenesulfonic acid monohydrate (269 mg, 1.4 mmol) and 3,4-dihydro-2H-pyran (1.3 mL, 14.0 mmol) in toluene (345 mL) was stirred at 60° C. for 16 h. The mixture was treated with EtOAc and washed with NaHCO_{3(aq)}. The organic layer was dried over Na₂SO₄₍ₛ₎, filtered, and concentrated under reduced pressure. The crude was purified by silica gel column chromatography (0-60% EtOAc in n-hexane) to give P50 (2.29 g, 82%) as a yellow oil. ¹H NMR (400 MHz, CDCl₃): δ 7.45 (d, J=8.8 Hz, 1H), 7.10-7.07 (m, 1H), 7.00 (d, J=2.0 Hz, 1H), 5.65-5.62 (m, 1H), 4.03-3.98 (m, 1H), 3.74-3.68 (m, 1H), 2.56-2.47 (m, 1H), 2.15-2.10 (m, 1H), 2.08-2.03 (m, 2H), 1.77-1.60 (m, 3H), 1.58 (s, 3H), 1.05-1.02 (m, 2H), 0.78-0.74 (m, 2H).

### Preparation 51. 2-Methyl-6-(5-(1-methylcyclopropoxy)-1-(tetrahydro-2H-pyran-2-yl)-1H-indazol-3-yl)-4-morpholinopyridazin-3(2H)-one (P51)

To a solution of P28 (crude, 1.74 mmol) in dimethoxyethane (15 mL) was added P50 (577 mg, 1.45 mmol) and potassium phosphate (2 M in water, 2.2 mL, 4.35 mmol). The mixture was degassed with argon for 15 min. The mixture was added by [1,1′-Bis(diphenylphosphino)ferrocene]dichloropalladium (II) complex with DCM (118 mg, 0.15 mmol) and degassed with argon again. The mixture was stirred at 90° C. for overnight. The mixture was cooled to room temperature and filtered with a pad of celite. The filtrate was concentrated in vacuo and then purified by silica gel chromatography (0-100% EtOAc in n-hexane). The resulting solid was washed with methanol to give P51 (943 mg) as a yellow solid. ¹H NMR (400 MHz, CDCl₃), δ: 8.05 (d, J=2.4 Hz, 1H), 7.48 (d, J=8.8 Hz, 1H), 7.29 (s, 1H), 7.10 (dd, J=8.8, 2.4 Hz, 1H), 5.68 (dd, J=9.4, 2.4 Hz, 1H), 4.10-3.84 (m, 8H), 3.77-3.72 (m, 1H), 3.54-3.52 (m, 4H), 2.65-2.55 (m, 1H), 2.13-2.05 (m, 2H), 1.80-1.72 (m, 3H), 1.61 (s, 3H), 1.09-1.05 (m, 2H), 0.76-0.72 (m, 2H).

### Synthesis of 2-methyl-6-(5-(1-methylcyclopropoxy)-1-(tetrahydro-2H-pyran-2-yl)-1H-indazol-3-yl)-4-morpholinopyridazin-3(2H)-one (P60)

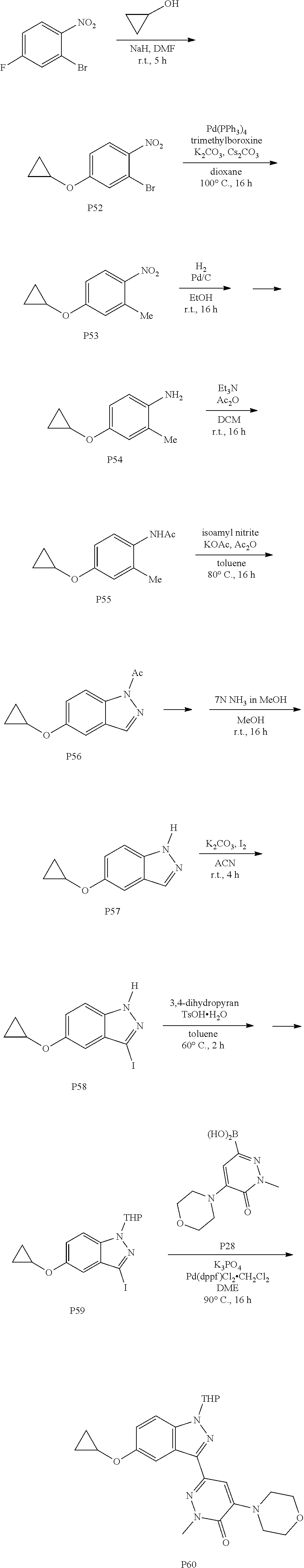

### Preparation 52. 2-Bromo-4-cyclopropoxy-1-nitrobenzene (P52)

To a solution of 60% sodium hydride in mineral oil (2.36 g, 59.1 mmol, 1.3 eq) in anh. DMF (100 mL) was added cyclopropanol (3.16 mL, 49.9 mmol, 1.1 eq) at 0° C. After the reaction mixture was stirred at 0° C. for 30 min, 2-bromo-4-fluoro-1-nitrobenzene (10.0 g, 45.4 mmol, 1.0 eq) was added at 0° C. After the reaction mixture was stirred at rt for 5 h, the solution was diluted with H₂O at 0° C. The mixture solution was extracted with EtOAc and washed with 1N HCl_{(aq)}. The organic layers were dried over MgSO₄₍ₛ₎, filtered, and concentrated under reduced pressure. The residue was purified by silica gel column chromatography (16% EtOAc in n-hexane) to afford P52 (4.17 g, 35%) as a yellow liquid. ¹H NMR (400 MHz, CDCl₃): δ 7.97 (d, J=9.2 Hz, 1H), 7.38 (d, J=2.8 Hz, 1H), 7.05 (dd, J=9.2, 2.8 Hz, 1H), 3.83-3.79 (m, 1H), 0.91-0.79 (m, 4H).

### Preparation 53. 4-Cyclopropoxy-2-methyl-1-nitrobenzene (P53)

A solution of P51 (4.45 g, 17.2 mmol, 1.0 eq) in 1,4-dioxane (170 mL) was added potassium carbonate (2.14 g, 34.5 mmol, 2.0 eq), cesium carbonate (2.52 g, 17.2 mmol, 1.0 eq) and 50% trimethylboroxine in THF (4.34 mL, 34.5 mmol, 2.0 eq). The reaction mixture was degassed with Argon and then tetrakis(triphenylphosphine)palladium(0) (895 mg, 1.72 mmol, 0.1 eq) was added. After the reaction solution was stirred at 100° C. for 16 h, the solution was filtered through a pad of celite and the filtrate was concentrated under reduced pressure. The crude was treated with water and extracted with EtOAc. The organic layers were dried over MgSO₄₍ₛ₎, filtered, and concentrated under reduced pressure. The residue was purified by silica gel column chromatography (10% EtOAc in n-hexane) to afford P53 (2.54 g, 76%) as a yellow liquid. ¹H NMR (400 MHz, CDCl₃), δ: 8.07 (d, J=9.2 Hz, 1H), 6.97-6.91 (m, 2H), 3.82-3.78 (m, 1H), 3.41 (s, 3H), 0.88-0.77 (m, 4H).

### Preparation 54. 4-Cyclopropoxy-2-methylaniline (P54)

To a solution of Pd/C (139 mg, 10% w/w) in ethanol (75 mL) was added a solution of P52 (1.39 g, 7.19 mmol, 1.0 eq) in ethanol (10 mL). After the solution was stirred at rt for 16 h under the hydrogen atmosphere, the reaction solution was filtered through a pad of Celite and washed with solvent. The filtrate was concentrated under reduced pressure. The residue was purified by silica gel column chromatography (16% EtOAc in n-hexane) to afford P54 (920 mg, 78%) as a pink liquid. ¹H NMR (400 MHz, CDCl₃), δ: 6.78-6.75 (m, 2H), 6.61 (d, J=8.4 Hz, 1H), 3.68-3.63 (m, 1H), 3.30 (br. s, 2H), 2.16 (s, 3H), 0.73-0.70 (m, 4H).

### Preparation 55. N-(4-cyclopropoxy-2-methylphenyl)acetamide (P55)

To a solution of P53 (920 mg, 5.63 mmol, 1.0 eq) in DCM (8.0 mL) was added triethylamine (0.864 mL, 6.20 mmol, 1.1 eq) and acetic anhydride (0.586 mL, 6.20 mmol, 1.1 eq) at 0° C. After the reaction solution was stirred at rt for 16 h, the solution was diluted with water and extracted with EtOAc. The organic layers were dried over MgSO₄₍ₛ₎, filtered, and concentrated under reduced pressure. The residue was purified by silica gel column chromatography (16% EtOAc in n-hexane) to afford P55 (1.06 g, 92%) as a white solid. ¹H NMR (400 MHz, CDCl₃), δ: 7.43 (d, J=8.4 Hz, 1H), 6.90-6.86 (m, 3H), 3.72-3.67 (m, 1H), 2.22 (s, 3H), 2.18 (s, 3H), 0.76-0.73 (m, 4H).

### Preparation 56. 1-(5-Cyclopropoxy-1H-indazol-1-yl)ethan-1-one (P56)

To a solution of P55 (1.06 g, 5.16 mmol, 1.0 eq) in toluene (60 mL) was added potassium acetate (760 mg, 7.75 mmol, 1.5 eq) and acetic anhydride (2.19 mL, 23.2 mmol, 4.5 eq) at room temperature. After the reaction solution was heated to 80° C., isoamyl nitrite (2.76 mL, 20.6 mmol, 4.0 eq) was added to the solution. The reaction solution was stirred at 80° C. for 16 h. The solution was diluted with water and extracted with EtOAc. The organic layers were dried over MgSO₄₍ₛ₎, filtered, and concentrated under reduced pressure. The residue was purified by silica gel column chromatography (16% EtOAc in n-hexane) to afford P56 (1.03 g, 92%) as an orange solid. ¹H NMR (400 MHz, CDCl₃), δ: 8.32 (d, J=9.2 Hz, 1H), 8.06 (s, 1H), 7.36 (d, J=2.4 Hz, 1H), 7.21 (dd, J=9.2, 2.4 Hz, 1H), 3.81-3.76 (m, 1H), 2.76 (s, 3H), 0.84-0.80 (m, 4H).

### Preparation 57. 5-Cyclopropoxy-1H-indazole (P57)

To a solution of P56 (1.03 g, 4.76 mmol, 1.0 eq) in methanol (20 mL) was added 7N ammonia in methanol (3.40 mL, 23.8 mmol, 5.0 eq). The reaction solution was stirred at rt for 16 h. The reaction solution was concentrated under reduced pressure to afford P57 (869 mg, 100%) as an orange solid which was used in the next step without purification. ¹H NMR (400 MHz, CDCl₃), δ: 8.00 (s, 1H), 7.40-7.37 (m, 2H), 7.07 (dd, J=8.8, 2.4 Hz, 1H), 3.79-3.75 (m, 1H), 0.82-0.79 (m, 4H).

### Preparation 58. 5-Cyclopropoxy-3-iodo-1H-indazole (P58)

To a solution of P57 (869 mg, 4.98 mmol, 1.0 eq) in acetonitrile (25 mL) was added potassium carbonate (2.06 g, 14.9 mmol, 3.0 eq) and iodine (2.53 g, 9.97 mmol, 2.0 eq). After the reaction solution was stirred at room temperature for 4 h, the mixture was diluted with water and extracted with EtOAc. The organic layers were washed with Na₂S₂O_{3(aq)}, dried over MgSO₄₍ₛ₎, filtered, and concentrated under reduced pressure. The residue was purified by silica gel column chromatography (25% EtOAc in n-hexane) to afford P58 (1.25 g, 83%) as a yellow solid. ¹H NMR (400 MHz, CDCl₃), δ: 10.32 (br. s, 1H), 7. 36 (dd, J=10.0 Hz, 1H), 7.12-7.11 (m, 2H), 3.84-3.81 (m, 1H), 0.88-0.79 (m, 4H).

### Preparation 59. 5-Cyclopropoxy-3-iodo-1-(tetrahydro-2H-pyran-2-yl)-1H-indazole (P59)

To a solution of P58 (1.25 g, 4.16 mmol, 1.0 eq) in toluene (20 mL) was added 3,4-dihydropyran (700 mg, 8.33 mmol, 2.0 eq) and p-toluenesulfonic acid monohydrate (158 mg, 0.833 mmol, 0.2 eq) at rt. After the reaction solution was stirred at 60° C. for 2 h, it was diluted with water and extracted with EtOAc. The organic layers were dried over MgSO₄₍ₛ₎, filtered, and concentrated under reduced pressure. The residue was purified by silica gel column chromatography (33% EtOAc in n-hexane) to afford P59 (1.66 g, 100%) as a yellow oil. ¹H NMR (400 MHz, CDCl₃), δ: 7.45 (d, J=9.2 Hz, 1H), 7.11-7.06 (m, 2H), 5.64 (dd, J=9.2, 2.8 Hz, 1H), 4.03-3.99 (m, 1H), 3.83-3.79 (m, 1H), 3.74-3.68 (m, 1H), 2.57-2.47 (m, 1H), 2.15-2.03 (m, 2H), 1.76-1.70 (m, 2H), 1.67-1.63 (m, 1H), 0.87-0.77 (m, 4H).

### Preparation 60. 6-(5-cyclopropoxy-1-(tetrahydro-2H-pyran-2-yl)-1H-indazol-3-yl)-2-methyl-4-morpholinopyridazin-3(2H)-one (P60)

To a crude solution of (1-methyl-5-morpholino-6-oxo-1,6-dihydropyridazin-3-yl)boronic acid (P28, 0.468 mmol) was added P59 (150 mg, 0.390 mmol, 1.0 eq), K₃PO_{4(aq)} (0.585 mL) and 1,2-dimethoxyethane (4.5 mL) at rt. The mixture solution was degassed with Argon_{(g)} and then added [1,1′-bis(diphenylphosphino)ferrocene]dichloropalladium(II), complex with dichloromethane (31.8 mg, 0.039 mmol, 0.1 eq). After the reaction solution was stirred at 90° C. for 16 h, the solution was filtered through a pad of Celite and washed with EtOAc. The filtrate solution was extracted with EtOAc. The organic layers were washed with water, dried over MgSO₄₍ₛ₎, filtered, and concentrated under reduced pressure. The residue was purified by silica gel column chromatography (50% EtOAc in n-hexane) to afford P60 (41 mg, 23%) as a brown solid. ¹H NMR (400 MHz, CDCl₃), δ: 8.09 (d, J=2.4 Hz, 1H), 7.49 (d, J=8.8 Hz, 1H), 7.30 (s, 1H), 7.13 (dd, J=9.2, 2.8 Hz, 1H), 5.69 (dd, J=9.6, 2.8 Hz, 1H), 4.11-4.07 (m, 1H), 3.91-3.89 (m, 7H), 3.85-3.81 (m, 1H), 3.79-3.73 (m, 1H), 3.55-3.53 (m, 4H), 2.86-2.57 (m, 1H), 2.19-2.16 (m, 1H), 2.07-2.04 (m, 1H) 1.81-1.76 (m, 2H), 1.67-1.66 (m, 1H), 0.84-0.82 (m, 4H).

### Synthesis of 4-((3aR,6aS)-3,3-dimethylhexahydro-1H-furo[3,4-b]pyrrol-1-yl)-2-methyl-6-(5-(1-methylcyclopropoxy)-1-(tetrahydro-2H-pyran-2-yl)-1H-indazol-3-yl)pyridazin-3(2H)-one (P60)

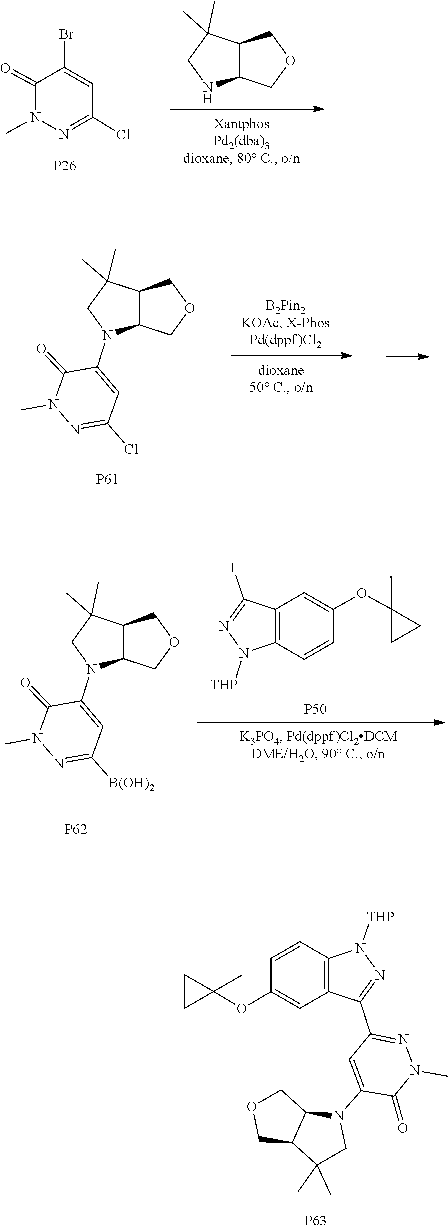

### Preparation 61. 6-Chloro-4-((3aR,6aS)-3,3-dimethylhexahydro-1H-furo[3,4-b]pyrrol-1-yl)-2-methylpyridazin-3(2H)-one (P61)

To a solution of P26 (250 mg, 1.12 mmol) in anhydrous 1,4-dioxane (7.5 mL) was added (3aR,6aS)-3,3-dimethyl-1,2,3a,4,6,6a-hexahydrofuro[3,4-b]pyrrole (190 mg, 1.34 mmol), cesium carbonate (729 mg, 2.24 mmol), and Xantphos (64.7 mg, 0.11 mmol). The mixture was degassed with argon for 15 min. The mixture was added by tris(dibenzylideneacetone)dipalladium(0) (51 mg, 0.06 mmol) and degassed with argon. After the mixture was stirred at 80° C. for overnight, the solution was filtered through a pad of celite. The filtrate was treated with water and extracted with EtOAc. The combined organic layers were dried over Na₂SO₄, filtered, and concentrated. The residue was purified by silica gel chromatography (0-40% EtOAc in n-hexane) to give P61 (69 mg, 28%) as a yellow solid. ¹H NMR (400 MHz, CD₃OD), δ: 6.09 (s, 1H), 4.85 (s, 2H), 3.93-3.89 (m, 1H), 3.80-3.71 (m, 2H), 3.66-3.64 (m, 1H), 3.61 (s, 3H), 3.28 (br. s, 1H), 2.59-2.53 (m, 1H), 1.13 (s, 3H), 1.12 (s, 3H); LCMS (ESI) m/z calc. for C₁₃H₁₈C₁N₃O₂ 283.11; found, 284.1 [M+H]⁺.

### Preparation 62. (5-((3aR,6aS)-3,3-dimethylhexahydro-1H-furo[3,4-b]pyrrol-1-yl)-1-methyl-6-oxo-1,6-dihydropyridazin-3-yl)boronic acid (P62)

To a solution of P61 (153 mg, 0.54 mmol) in anh. 1,4-dioxane (5.4 mL) was added bis(pinacolato)diboron (684 mg, 2.70 mmol), potassium acetate (106 mg, 1.08 mmol), XPhos (5.1 mg, 0.11 mmol). The mixture was bubbled with argon for 15 min. The mixture was added by [1,1′-bis(diphenylphosphino)ferrocene]dichloropalladium(II) (39 mg, 0.05 mmol) and then bubbled with argon for 15 min. After the mixture was stirred at 50° C. for overnight, the solution was used in the next step without further purification. LCMS (ESI) m/z calc. for C₁₃H₂₀BN₃O₄ 293.15; found, 294.2 [M+H]⁺.

### Preparation 63. 4-((3aR,6aS)-3,3-dimethylhexahydro-1H-furo[3,4-b]pyrrol-1-yl)-2-methyl-6-(5-(1-methylcyclopropoxy)-1-(tetrahydro-2H-pyran-2-yl)-1H-indazol-3-yl)pyridazin-3(2H)-one (P63)

To a solution of P62 (crude, 0.54 mmol), P50 (150 mg, 0.38 mmol) and potassium phosphate (242 mg, 1.14 mmol, 2 M in water) in 1,2-dimethoxyethane (3.8 mL) was degassed with argon for 15 min. The mixture was added by [1,1′-bis(diphenylphosphino)ferrocene]dichloropalladium(II) dichloromethane complex (33 mg, 0.04 mmol) and then degassed with argon for 15 min. After the mixture was stirred at 90° C. overnight, the mixture was filtered through a pad of celite. The filtrate was concentrated in vacuo and then purified by silica gel chromatography (0-50% EtOAc in n-hexane) to give P63 (69 mg, 34%) as a yellow oil. ¹H (399 MHz, CD₃OD), δ: 8.07 (d, J=2.4 Hz, 1H), 7.56 (d, J=9.2 Hz, 1H), 7.07 (dd, J=9.2, 2.4 Hz, 1H), 6.96 (s, 1H), 5.79-5.77 (m, 1H), 5.31 (br. s, 1H), 4.61 (br. s, 2H), 4.03-3.99 (m, 1H), 3.96-3.92 (m, 1H), 3.84 (s, 3H), 3.79-3.77 (m, 3H), 3.72-3.69 (m, 1H), 3.38 (br. s, 2H), 2.60-2.49 (m, 2H), 2.14-2.11 (m, 1H), 2.07-2.18 (m, 1H), 1.87-1.67 (m, 3H), 1.60 (s, 3H), 1.13-1.12 (m, 6H), 1.04-1.00 (m, 2H), 0.80-0.77 (m, 2H); LCMS (ESI) m/z calc. for C₂₉H₃₇N₅O₄ 519.28; found, 520.4 [M+H]⁺.

### Synthesis of 4-((2S,6R)-2,6-dimethylmorpholino)-2-methyl-6-(5-(1-methylcyclopropoxy)-1-(tetrahydro-2H-pyran-2-yl)-1H-indazol-3-yl)pyridazin-3(2H)-one (P60)

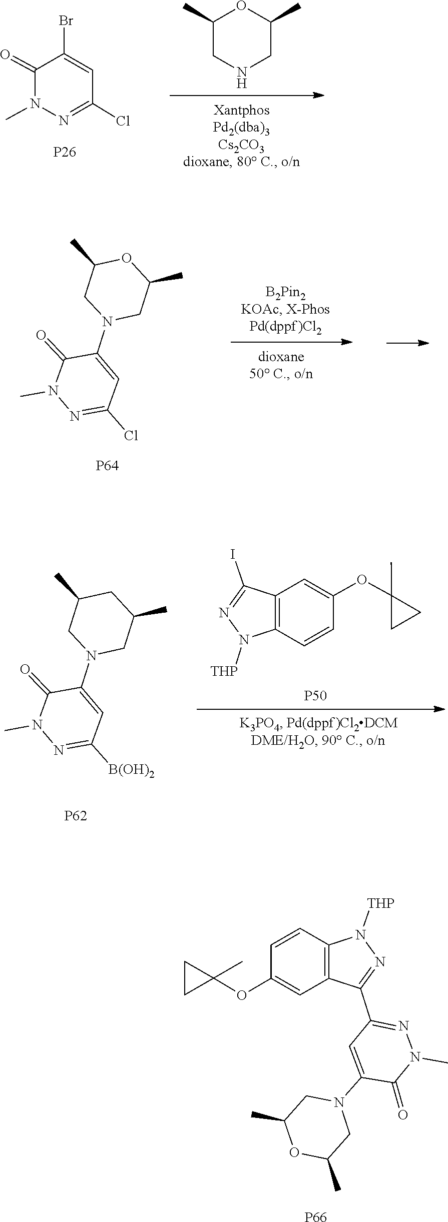

### Preparation 64. 6-Chloro-4-((2S,6R)-2,6-dimethylmorpholino)-2-methylpyridazin-3(2H)-one (P64)

To a solution of P26 (1 g, 4.48 mmol) in anhydrous 1,4-dioxane (30 mL) was added cis-2,6-dimethylmorpholine (0.6 mL, 4.92 mmol), cesium carbonate (2.9 g, 8.96 mmol), and Xantphos (259 mg, 0.45 mmol). The mixture was degassed with argon for 15 min. The mixture was added by tris(dibenzylideneacetone)dipalladium(0) (205 mg, 0.22 mmol) and degassed with argon again. After the mixture was stirred at 80° C. for overnight, the solution was filtered through a pad of celite. The filtrate was treated with water and extracted with EtOAc. The combined organic layers were dried over Na₂SO₄, filtered, and concentrated. The residue was purified by silica gel chromatography (0-40% EtOAc in n-hexane) to give P64 (472 mg, 40%) as a yellow solid. ¹H NMR (400 MHz, CD₃OD), δ: 6.22 (s, 1H), 4.12-4.09 (m, 2H), 3.84-3.76 (m, 2H), 3.70 (s, 3H), 2.50-2.45 (m 2H), 1.22 (d, J=6.0 Hz, 6H).

### Preparation 65. (5-((2S,6R)-2,6-dimethylmorpholino)-1-methyl-6-oxo-1,6-dihydropyridazin-3-yl)boronic acid (P65)

To a solution of P64 (200 mg, 0.78 mmol) in anhydrous 1,4-dioxane (7.8 mL) was added bis(pinacolato)diboron (990 mg, 3.90 mmol), potassium acetate (153 mg, 1.56 mmol), XPhos (75 mg, 0.16 mmol). The mixture was degassed with argon for 15 min. The mixture was added by [1,1′-bis(diphenylphosphino)ferrocene]dichloropalladium(II) (57 mg, 0.08 mmol) and then degassed with argon for 15 min. After the mixture was stirred at 50° C. for overnight, the solution was used in the next step without further purification. LCMS (ESI) m/z calc. for C₁₃H₂₀BN₃O₄ 293.15; found, 294.2 [M+H]⁺.

### Preparation 66. 4-((2S,6R)-2,6-dimethylmorpholino)-2-methyl-6-(5-(1-methylcyclopropoxy)-1-(tetrahydro-2H-pyran-2-yl)-1H-indazol-3-yl)pyridazin-3(2H)-one (P66)

To a above crude solution of P65 (0.78 mmol), P50 (259 mg, 0.65 mmol) and potassium phosphate (413 mg, 1.95 mmol, 2 M in water) in 1,2-dimethoxyethane (6.5 mL) was degassed with argon for 15 min. The mixture was added by [1,1′-bis(diphenylphosphino)ferrocene]dichloropalladium(II) dichloromethane complex (64 mg, 0.08 mmol) and then degassed with argon for 15 min. After the mixture was stirred at 90° C. overnight, the mixture was filtered through a pad of celite. The filtrate was concentrated in vacuo and then purified by silica gel chromatography (0-50% EtOAc in n-hexane) to give P66 (423 mg, 99%) as a yellow solid. ¹H (400 MHz, CDCl₃), δ: 8.06 (d, J=2.4 Hz, 1H), 7.48 (d, J=9.2 Hz, 1H), 7.11 (dd, J=9.2, 2.4 Hz, 1H), 6.98 (s, 1H), 5.69 (dd, J=9.9, 2.6 Hz, 1H), 4.64 (br. s, 2H), 4.17-4.08 (m, 3H), 3.94-3.85 (m, 4H), 3.79-3.73 (m, 2H), 2.53-2.44 (m, 2H), 2.06-2.00 (m, 4H), 1.61 (s, 3H), 1.29-1.28 (m, 6H), 1.10-1.08 (m, 2H), 0.76-0.73 (m, 2H).

### Synthesis of 2-cyclopropyl-4-((2S,6R)-2,6-dimethylmorpholino)-6-(5-(1-methylcyclopropoxy)-1-(tetrahydro-2H-pyran-2-yl)-1H-indazol-3-yl)pyridazin-3(2H)-one (P69)

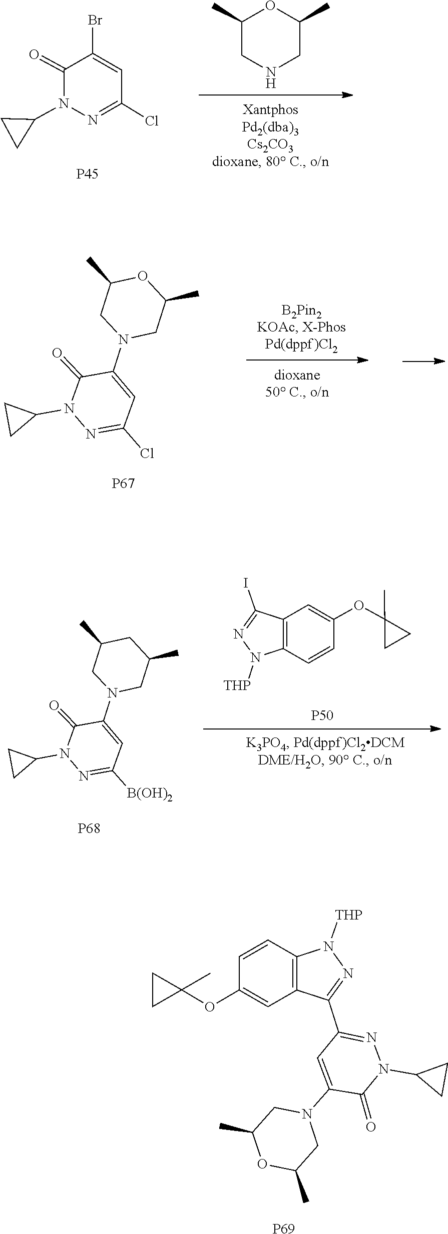

### Preparation 67. 6-Chloro-2-cyclopropyl-4-((2S,6R)-2,6-dimethylmorpholino)pyridazin-3(2H)-one (P67)

To a solution of P45 (300 mg, 1.20 mmol) in anh. 1,4-dioxane (8.6 mL) was added cis-2,6-dimethylmorpholine (148 μL, 1.20 mmol), cesium carbonate (782 mg, 2.40 mmol), and Xantphos (69 mg, 0.12 mmol). The mixture was degassed with argon for 15 min. The mixture was added by tris(dibenzylideneacetone)dipalladium(0) (55 mg, 0.06 mmol) and degassed with argon. After the mixture was stirred at 80° C. for overnight, the solution was filtered through a pad of celite. The filtrate was treated with water and extracted with EtOAc. The combined organic layers were dried over Na₂SO₄, filtered, and concentrated. The residue was purified by silica gel chromatography (0-40% EtOAc in n-hexane) to give P67 (50 mg, 14%) as a yellow solid. ¹H NMR (400 MHz, CDCl₃), δ: 6.19 (s, 1H), 4.11-4.09 (m, 2H), 3.92-3.85 (m, 1H), 3.85-3.77 (m, 2H), 2.49-2.43 (m, 2H), 1.23 (s, 3H), 1.21 (s, 3H), 1.11-1.07 (m, 2H), 1.01-0.96 (m, 2H).

### Preparation 68. (1-Cyclopropyl-5-((2S,6R)-2,6-dimethylmorpholino)-6-oxo-1,6-dihydropyridazin-3-yl) boronic acid (P68)

To a solution of P67 (90 mg, 0.32 mmol) in anh. 1,4-dioxane (3.2 mL) was added bis(pinacolato)diboron (403 mg, 1.59 mmol), potassium acetate (63 mg, 0.64 mmol), XPhos (31 mg, 0.06 mmol). The mixture was degassed with argon for 15 min. The mixture was added by [1,1′-bis(diphenylphosphino)ferrocene]dichloropalladium(II) (23 mg, 0.03 mmol) and then degassed with argon for 15 min. After the mixture was stirred at 50° C. for overnight, the solution was used in the next step without further purification. LCMS (ESI) m/z calc. for C₁₃H₂₀BN₃O₄ 293.15; found, 294.2 [M+H]⁺.

### Preparation 69. 2-Cyclopropyl-4-((2S,6R)-2,6-dimethylmorpholino)-6-(5-(1-methylcyclopropoxy)-1-(tetrahydro-2H-pyran-2-yl)-1H-indazol-3-yl)pyridazin-3(2H)-one (P69)

To a crude solution of P68 (0.32 mmol) was added P50 (106 mg, 0.27 mmol) potassium phosphate (172 mg, 0.81 mmol, 2 M in water), and 1,2-dimethoxyethane (3.0 mL), and it was degassed with argon for 15 min. The mixture was added by [1,1′-bis(diphenylphosphino)ferrocene]dichloropalladium(II) dichloromethane complex (26 mg, 0.03 mmol) and then degassed with argon for 15 min. After the mixture was stirred at 90° C. overnight, the mixture was filtered through a pad of celite. The filtrate was concentrated in vacuo and then purified by silica gel chromatography (0-40% EtOAc in n-hexane) to give P69 (130 mg, 99%) as a yellow solid. ¹H (400 MHz, CDCl₃), δ: 7.94 (d, J=2.4 Hz, 1H), 7.47 (d, J=9.0 Hz, 1H), 7.03 (dd, J=9.0, 2.4 Hz, 1H), 6.97 (s, 1H), 5.69-5.66 (m, 1H), 4.36-4.30 (m, 1H), 4.17-4.08 (m, 2H), 4.03-4.00 (m, 4H), 3.99-3.93 (m, 2H), 3.87-3.81 (m, 4H), 2.43-2.36 (m, 2H), 1.64 (s, 3H), 1.31-1.30 (m, 6H), 0.95-0.92 (m, 8H).

### Synthesis of 2-methyl-6-(5-(1-methylcyclopropoxy)-1-(tetrahydro-2H-pyran-2-yl)-1H-indazol-3-yl)-4-(5-oxa-8-azaspiro[2.6]nonan-8-yl)pyridazin-3(2H)-one (P73)

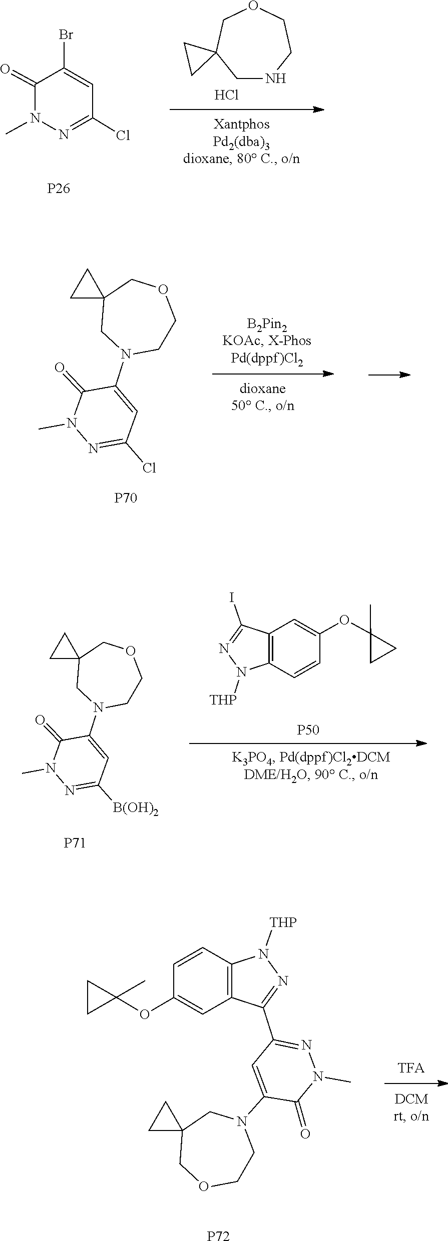

### Preparation 70. 6-Chloro-2-methyl-4-(5-oxa-8-azaspiro[2.6]nonan-8-yl)pyridazin-3(2H)-one (P70)

To a solution of P26 (100 mg, 0.45 mmol) in anhydrous 1,4-dioxane (3 mL) was added 5-oxa-8-azaspiro[2.6]nonane hydrochloride (88 mg, 0.54 mmol), cesium carbonate (440 mg, 1.35 mmol), and Xantphos (29 mg, 0.05 mmol). The mixture was degassed with argon for 15 min. The mixture was added by tris(dibenzylideneacetone)dipalladium(0) (21 mg, 0.02 mmol) and degassed with argon. After the mixture was stirred at 80° C. for overnight, the solution was filtered through a pad of celite. The filtrate was treated with water and extracted with EtOAc. The combined organic layers were dried over Na₂SO₄, filtered, and concentrated. The residue was purified by silica gel chromatography (0-40% EtOAc in n-hexane) to give P70 (71 mg, 58%) as a yellow solid. ¹H NMR (400 MHz, CDCl₃), δ: 6.01 (s, 1H), 4.10-4.06 (m, 2H), 3.90-3.88 (m, 2H), 3.64 (s, 3H), 3.48-3.44 (m, 4H), 0.70 (br m, 2H), 0.62 (br. m, 2H).

### Preparation 71. (1-Methyl-6-oxo-5-(5-oxa-8-azaspiro[2.6]nonan-8-yl)-1,6-dihydropyridazin-3-yl)boronic acid (P71)

To a solution of P70 (137 mg, 0.51 mmol) in anh. 1,4-dioxane (5 mL) was added bis(pinacolato)diboron (645 mg, 2.54 mmol), potassium acetate (100 mg, 1.02 mmol), XPhos (48.6 mg, 0.10 mmol). The mixture was degassed with argon for 15 min. The mixture was added by [1,1′-bis(diphenylphosphino)ferrocene]dichloropalladium(II) (37 mg, 0.05 mmol) and then degassed with argon for 15 min. After the mixture was stirred at 50° C. for overnight, the solution was used in the next step without further purification. LCMS (ESI) m/z calc. for C₁₂H₁₈BN₃O₄ 279.14; found, 280.1 [M+H]⁺.

### Preparation 72. 2-Methyl-6-(5-(1-methylcyclopropoxy)-1-(tetrahydro-2H-pyran-2-yl)-1H-indazol-3-yl)-4-(5-oxa-8-azaspiro[2.6]nonan-8-yl)pyridazin-3(2H)-one (P72)

To a crude solution of P71 (crude, 0.51 mmol) was added P50 (169 mg, 0.43 mmol), potassium phosphate (273 mg, 1.29 mmol, 2 M in water) and 1,2-dimethoxyethane (4.3 mL), and it was degassed with argon for 15 min. The mixture was added by [1,1′-bis(diphenylphosphino)ferrocene]dichloropalladium(II) dichloromethane complex (35 mg, 0.04 mmol) and then degassed with argon for 15 min. After the mixture was stirred at 90° C. overnight, the mixture was filtered through a pad of celite. The filtrate was concentrated in vacuo and then purified by silica gel chromatography (0-70% EtOAc in n-hexane) to give P72 (160 mg, 62%) as a yellow oil. ¹H (400 MHz, CD₃OD), δ: 8.07 (d, J=2.4 Hz, 1H), 7.57 (d, J=9.0, Hz, 1H), 7.24 (s, 1H), 7.08 (dd, J=9.0, 2.4 Hz, 1H), 5.79 (dd, J=9.6, 2.4 Hz, 1H), 4.15-4.11 (m, 2H), 4.02-3.99 (m, 1H), 3.94-3.92 (m, 2H), 3.85 (s, 3H), 3.82-3.78 (m, 1H), 3.58 (s, 2H), 3.52 (s, 2H), 2.59-2.49 (m, 1H), 2.17-2.13 (m, 1H), 2.07-2.02 (m, 1H), 1.90-1.80 (m, 1H), 1.75-1.65 (m, 2H), 1.60 (s, 3H), 1.04-1.00 (m, 2H), 0.80-0.77 (m, 2H), 0.73-0.69 (m, 4H); LCMS (ESI) m/z calc. for C₂₈H₃₅N₅O₄ 505.27; found, 506.4 [M+H]⁺.

### Synthesis of 4-(3,3-difluoropiperidin-1-yl)-2-methyl-6-(5-(1-methylcyclopropoxy)-1-(tetrahydro-2H-pyran-2-yl)-1H-indazol-3-yl)pyridazin-3(2H)-one (P73)

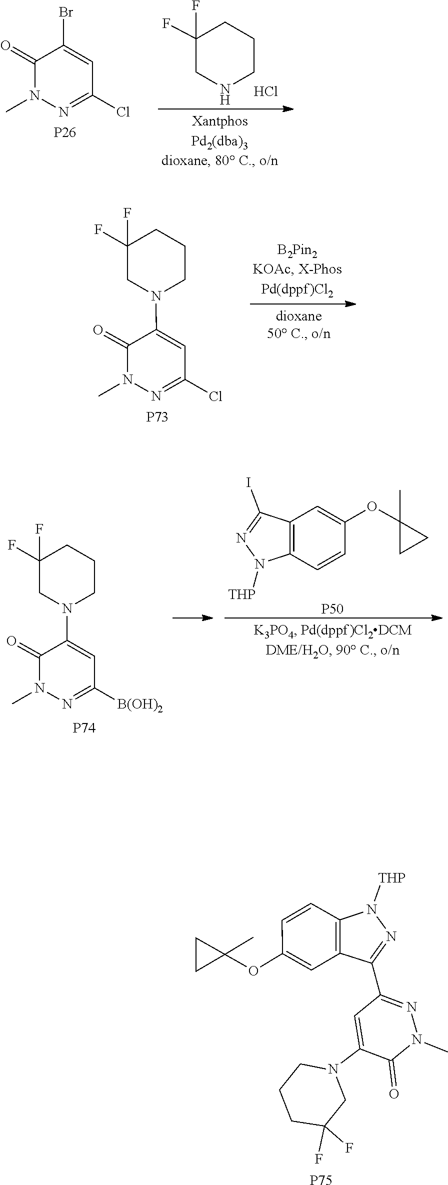

### Preparation 73. 6-Chloro-4-(3,3-difluoropiperidin-1-yl)-2-methylpyridazin-3(2H)-one (P73)

To a solution of P26 (500 mg, 2.25 mmol) in anh. 1,4-dioxane (15 mL) was added 3,3-difluoropiperidine hydrochloride (390 mg, 2.50 mmol), cesium carbonate (2.19 g, 6.75 mmol), and Xantphos (130 mg, 0.23 mmol). The mixture was degassed with argon for 15 min. The mixture was added by tris(dibenzylideneacetone)dipalladium(0) (105 mg, 0.11 mmol) and degassed with argon. After the mixture was stirred at 80° C. for overnight, the solution was filtered through a pad of celite. The filtrate was treated with water and extracted with EtOAc. The combined organic layers were dried over Na₂SO₄, filtered, and concentrated. The residue was purified by silica gel chromatography (0-25% EtOAc in n-hexane) to give P73 (71 mg, 12%) as a yellow solid. ¹H NMR (400 MHz, CDCl₃), δ: 6.29 (s, 1H), 3.99 (t, ³J_{H-F}=11.6 Hz, 2H), 3.70 (s, 3H), 3.54-3.49 (m, 2H), 2.14-2.04 (m, 2H), 1.94-1.86 (m, 2H).

### Preparation 74. (5-(3,3-Difluoropiperidin-1-yl)-1-methyl-6-oxo-1,6-dihydropyridazin-3-yl)boronic acid (P74)

To a solution of P73 (86 mg, 0.33 mmol) in anh. 1,4-dioxane (3.0 mL) was added bis(pinacolato)diboron (418 mg, 1.65 mmol), potassium acetate (65 mg, 0.66 mmol), XPhos (33 mg, 0.07 mmol). The mixture was degassed with argon for 15 min. The mixture was added by [1,1′-bis(diphenylphosphino)ferrocene]dichloropalladium(II) (22 mg, 0.03 mmol) and then degassed with argon for 15 min. After the mixture was stirred at 50° C. for overnight, the solution was used in the next step without further purification. LCMS (ESI) m/z calc. for C₁₀H₁₄BF₂N₃O₃ 273.11; found, 274.1 [M+H]⁺.

### Preparation 75. 4-(3,3-difluoropiperidin-1-yl)-2-methyl-6-(5-(1-methylcyclopropoxy)-1-(tetrahydro-2H-pyran-2-yl)-1H-indazol-3-yl)pyridazin-3(2H)-one (P75)

A solution of P74 (crude, 0.33 mmol), P50 (109 mg, 0.28 mmol) and potassium phosphate (178 mg, 0.84 mmol, 2 M in water) in 1,2-dimethoxyethane (3 mL) was degassed with argon for 15 min. The mixture was added by [1,1′-bis(diphenylphosphino)ferrocene]dichloropalladium(II) dichloromethane complex (24 mg, 0.03 mmol) and then degassed with argon for 15 min. After the mixture was stirred at 90° C. overnight, the mixture was filtered through a pad of celite. The filtrate was concentrated in vacuo and then purified by silica gel chromatography (0-25% EtOAc in n-hexane) to give P75 (93 mg, 57%, 2 steps) as a yellow solid. ¹H (400 MHz, CDCl₃), δ: 8.05 (d, J=2.4 Hz, 1H), 7.48 (d, J=9.6 Hz, 1H), 7.32 (s, 1H), 7.10 (dd, J=9.6, 2.4 Hz, 1H), 5.69 (dd, J=9.6, 2.8 Hz, 1H), 5.09 (s, 1H), 4.09-4.06 (m, 1H), 3.96-3.88 (m, 6H), 3.80-3.71 (m, 2H), 3.62-3.46 (m, 3H), 2.65-2.56 (m, 1H), 1.80-1.74 (m, 3H), 1.61 (s, 3H), 1.09-1.05 (m, 2H), 0.76-0.72 (m, 2H); LCMS (ESI) m/z calc. for C₂₆H₃₁F₂N₅O₃ 499.24; found, 500.3 [M+H]⁺.

### Synthesis of 4-(3,3-difluoropiperidin-1-yl)-2-methyl-6-(5-(1-methylcyclopropoxy)-1-(tetrahydro-2H-pyran-2-yl)-1H-indazol-3-yl)pyridazin-3(2H)-one (P73)

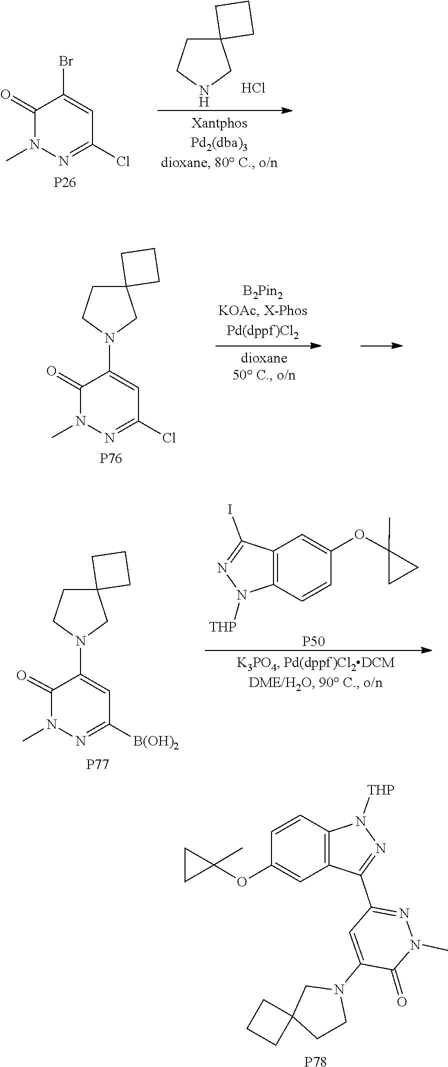

### Preparation 76. 6-Chloro-2-methyl-4-(6-azaspiro[3.4]octan-6-yl)pyridazin-3(2H)-one (P76)

To a solution of P26 (100 mg, 0.45 mmol) in anh. 1,4-dioxane (3.0 mL) was added 6-azaspiro[3.4]octane hydrochloride (73 mg, 0.50 mmol), cesium carbonate (437 mg, 1.35 mmol), and Xantphos (26 mg, 0.04 mmol). The mixture was degassed with argon for 15 min. The mixture was added by tris(dibenzylideneacetone)dipalladium(0) (21 mg, 0.02 mmol) and degassed with argon. After the mixture was stirred at 80° C. for overnight, the solution was filtered through a pad of celite. The filtrate was treated with water and extracted with EtOAc. The combined organic layers were dried over Na₂SO₄, filtered, and concentrated. The residue was purified by silica gel chromatography (0-80% EtOAc in n-hexane) to give P76 (100 mg, 88%) as a yellow solid. ¹H NMR (400 MHz, CD₃OD), δ: 5.97 (s, 1H), 4.86 (s, 4H), 3.59 (s, 3H), 2.10-1.88 (m, 8H).

### Preparation 77. (1-Methyl-6-oxo-5-(6-azaspiro[3.4]octan-6-yl)-1,6-dihydropyridazin-3-yl)boronic acid (P77)

To a solution of P76 (100 mg, 0.39 mmol) in anh. 1,4-dioxane (4 mL) was added bis(pinacolato)diboron (500 mg, 1.97 mmol), potassium acetate (77.3 mg, 0.79 mmol), XPhos (38 mg, 0.08 mmol). The mixture was degassed with argon for 15 min. The mixture was added by [1,1′-bis(diphenylphosphino)ferrocene]dichloropalladium(II) (29 mg, 0.04 mmol) and then degassed with argon for 15 min. After the mixture was stirred at 50° C. for overnight, the solution was used in the next step without further purification. LCMS (ESI) m/z calc. for C₁₂H₁₈BN₃O₃ 263.14; found, 264.2 [M+H]⁺.

### Preparation 78. 2-Methyl-6-(5-(1-methylcyclopropoxy)-1-(tetrahydro-2H-pyran-2-yl)-1H-indazol-3-yl)-4-(6-azaspiro[3.4]octan-6-yl)pyridazin-3(2H)-one (P78)

A solution of P77 (crude, 0.39 mmol), P50 (129 mg, 0.32 mmol) and potassium phosphate (206 mg, 0.98 mmol, 2 M in water) in 1,2-dimethoxyethane (3 mL) was degassed with argon for 15 min. The mixture was added by [1,1′-bis(diphenylphosphino)ferrocene]dichloropalladium(II) dichloromethane complex (26.5 mg, 0.03 mmol) and then degassed with argon for 15 min. After the mixture was stirred at 90° C. overnight, the mixture was filtered through a pad of celite. The filtrate was concentrated in vacuo and then purified by silica gel chromatography (0-40% EtOAc in n-hexane) to give P78 (72.3 mg, 37%, 2 steps) as a yellow solid. ¹H (400 MHz, CDCl₃), δ: 8.06 (d, J=2.4 Hz, 1H), 7.45 (d, J=9.0 Hz, 1H), 7.07 (dd, J=9.0, 2.4 Hz, 1H), 6.96 (s, 1H), 6.73 (s, 1H), 5.68-5.65 (m, 1H), 4.07-4.04 (m, 1H), 3.82 (s, 3H), 3.80-3.77 (m, 1H), 3.74-3.69 (m, 2H), 2.92-2.55 (m, 1H), 2.41-2.36 (m, 1H), 2.04-2.02 (m, 2H), 1.95-1.90 (m, 5H), 1.78-1.66 (m, 2H), 1.59 (s, 3H), 1.14-1.12 (m, 2H), 1.07-1.04 (m, 2H), 0.93-0.91 (m, 2H), 0.74-0.70 (m, 2H); LCMS (ESI) m/z calc. for C₂₈H₃₅N₅O₃ 489.27; found, 490.4 [M+H]⁺.

### Synthesis of 2-cyclopentyl-6-(5-isopropoxy-1-trityl-1H-indazol-3-yl)-4-morpholinopyridazin-3(2H)-one (P82)

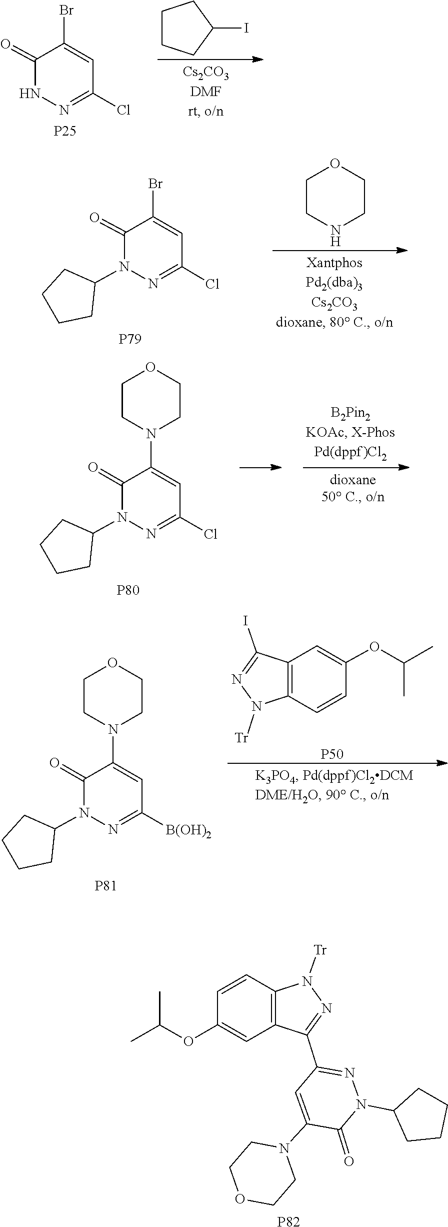

### Preparation 79. 4-Bromo-6-chloro-2-cyclopentylpyridazin-3(2H)-one (P79)

To a solution of P25 (1.0 g, 4.8 mmol) and cesium carbonate (2.3 g, 7.2 mmol) in DMF (10 mL) was added iodocyclopentane (1.4 g, 7.2 mmol) in an ice bath. The reaction was stirred at rt for overnight. The mixture was treated with water and extracted with EtOAc. The combined organic layers were dried over Na₂SO₄, filtered, and concentrated. The residue was purified by silica gel chromatography (0-30% EtOAc in n-hexane) to give P79 (1.15 g) as a yellow oil. ¹H NMR (400 MHz, CDCl₃), δ: 7.57 (s, 1H), 5.38-5.31 (m, 1H), 2.09-2.0 (m, 2H), 1.88-1.83 (m, 4H), 1.68-1.63 (m, 2H).

### Preparation 80. 6-Chloro-2-cyclopentyl-4-morpholinopyridazin-3(2H)-one (P81)

To a solution of P79 (500 mg, 1.8 mmol) in anh. 1,4-dioxane (15 mL) was added morpholine (157 μL, 1.80 mmol), cesium carbonate (1.27 g, 3.6 mmol), and Xantphos (104.3 mg, 0.18 mmol). The mixture was degassed by argon for 15 min. The mixture was added by tris(dibenzylideneacetone)dipalladium(0) (80 mg, 0.1 mmol) and was degassed with argon again. After the mixture was stirred at 80° C. for overnight, the solution was filtered through a pad of celite. The filtrate was treated with water and extracted with EtOAc. The combined organic layers were dried over Na₂SO₄, filtered, and concentrated. The residue was purified by silica gel chromatography (0-20% EtOAc in n-hexane) to give P80 (450 mg) as an orange oil. ¹H NMR (400 MHz, CDCl₃), δ: 6.20 (s, 1H), 5.34-5.27 (m, 1H), 3.84-3.82 (m, 4H), 3.47-3.45 (m, 4H), 2.04-1.97 (m, 2H), 1.91-1.81 (m, 4H), 1.67-1.59 (m, 2H).

### Preparation 81. (1-Cyclopentyl-5-morpholino-6-oxo-1,6-dihydropyridazin-3-yl)boronic acid (P81)

To a solution of P80 (450 mg, 1.59 mmol) in anh. 1,4-dioxane (16 mL) was added bis(pinacolato)diboron (2.01 g, 7.93 mmol), potassium acetate (312 mg, 3.18 mmol), XPhos (152 mg, 0.32 mmol). The mixture was degassed by argon for 15 min. The mixture was added by [1,1′-bis(diphenylphosphino)ferrocene]dichloropalladium(II) (117 mg, 0.16 mmol) and then was degassed by argon for 15 min again. After the mixture was stirred at 50° C. for 6 h, the solution was filtered through a pad of celite and the filtrate was concentrated to give P81 (473 mg) as an orange oil which was used in next step without further purification. LCMS (ESI) m/z calc. for C₁₃H₂₀BN₃O₄ 293.15; found, 293.8 [M+H]⁺.

### Preparation 82. 2-Cyclopentyl-6-(5-isopropoxy-1-trityl-1H-indazol-3-yl)-4-morpholinopyridazin-3(2H)-one (P82)

To a solution of P81 (crude, 1.59 mmol) in dimethoxyethane (16 mL) was added P50 (721 mg, 1.33 mmol) and potassium phosphate (2 M in water, 2 mL, 3.99 mmol). The mixture was degassed by argon for 15 min. The mixture was added by [1,1′-Bis(diphenylphosphino)ferrocene]dichloropalladium(II) dichloromethane complex (109 mg, 0.13 mmol) and the solution was degassed by argon. The mixture was stirred at 90° C. for overnight and then cooled to rt. The solution was filtered through a pad of celite. The filtrate was concentrated in vacuo and then purified by silica gel chromatography (0-100% EtOAc in n-hexane). The resulting solid was washed with methanol to give P82 (1.2 g) as a yellow solid. ¹H NMR (400 MHz, CDCl₃), δ: 7.78 (d, J=2.4 Hz, 1H), 7.28-7.27 (m, 8H), 7.18-7.16 (m, 7H), 7.06 (s, 1H), 6.63 (dd, J=9.2, 2.4 Hz, 1H), 6.21 (d, J=9.2 Hz, 1H), 5.58-5.50 (m, 1H), 4.56 (septet, J=6.0 Hz, 1H), 3.88-3.83 (m, 4H), 3.39-3.37 (m, 4H), 2.19-2.06 (m, 4H), 1.76-1.72 (m, 4H), 1.36 (d, J=6.0 Hz, 6H).

### Synthesis of 2-cyclopentyl-6-(5-isopropoxy-1-trityl-1H-indazol-3-yl)-4-morpholinopyridazin-3(2H)-one (P85)

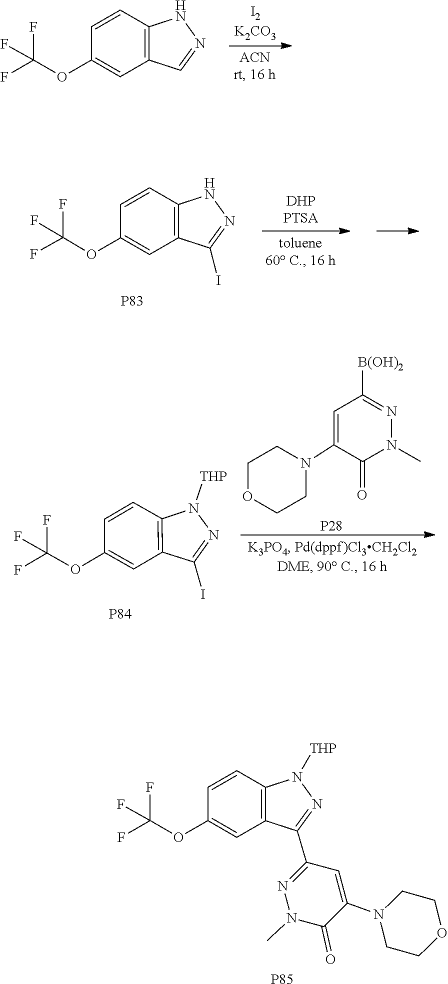

### Preparation 83. 3-Iodo-5-(trifluoromethoxy)-1H-indazole (P83)

To a solution of 5-(trifluoromethoxy)-1H-indazole (1.00 g, 4.95 mmol) in acetonitrile (20 mL) was added iodine (1.88 g, 7.42 mmol) and potassium carbonate (1.37 g, 9.89 mmol). After the mixture was stirred at rt for overnight, the mixture was treated with saturated sodium thiosulfate (10 mL) and extracted with EtOAc. The combined organic layers were dried over MgSO₄, filtered, and concentrated. The residue was purified by silica gel chromatography (0-30% EtOAc in n-hexane) to give P83 (1.50 g, 99%) as a yellow solid. ¹H NMR (400 MHz, CDCl₃), δ: 10.68 (br. s, 1H), 7.55-7.50 (m, 1H), 7.41-7.32 (m, 2H); LCMS (ESI) m/z calc. for C₈H₄F₃IN₂O 327.93; found, 328.7 [M+H]⁺.

### Preparation 84. 3-Iodo-1-(tetrahydro-2H-pyran-2-yl)-5-(trifluoromethoxy)-1H-indazole (P84)

To a solution of P83 (1.40 g, 4.27 mmol) in toluene (17 mL) was added 3,4-dihydro-2H-pyran (0.780 mL, 8.54 mmol) and p-toluenesulfonic acid monohydrate (162 mg, 0.854 mmol) at 0° C. After the mixture was stirred at rt overnight, the mixture was treated with water (20 mL) and extracted with EtOAc. The combined organic layers were dried over MgSO₄, filtered, and concentrated. The residue was purified by silica gel chromatography (0-20% EtOAc in n-hexane) to give P84 (1.43 g, 81%) as a white solid. ¹H NMR (400 MHz, CDCl₃), δ: 7.63-7.58 (m, 1H), 7.35-7.29 (m, 2H), 5.70 (dd, J=8.8, 2.8 Hz, 1H), 4.04-3.95 (m, 1H), 3.78-3.68 (m, 1H), 2.58-2.44 (m, 1H), 2.21-2.02 (m, 2H), 1.83-1.61 (m, 3H); LCMS (ESI) m/z calc. for C₁₃H₁₂F₃IN₂O₂ 411.99; found, 412.7 [M+H]⁺.

### Preparation 85. 2-Methyl-4-morpholino-6-(1-(tetrahydro-2H-pyran-2-yl)-5-(trifluoromethoxy)-1H-indazol-3-yl)pyridazin-3(2H)-one (P85)

To a solution of P28 (crude, 1.31 mmol) in 1,2-dimethoxyethane (12 mL) was added P84 (448 mg, 1.09 mmol) and potassium phosphate (2M in H₂O solution, 1.60 mL, 3.26 mmol). The mixture was degassed by argon for 15 min. [1,1′-Bis(diphenylphosphino)ferrocene]dichloropalladium(II) complex with dichloromethane (89 mg, 0.11 mmol) was added to the mixture, and it was degassed with argon again. After the mixture was stirred at 90° C. for overnight, the solution was diluted with EtOAc and was filtered through a pad of celite. The filtrate was concentrated in vacuo and then purified by silica gel chromatography (0-30% EtOAc in n-hexane) to give P85 (800 mg, N1/N2 isomers) as a white solid. ¹H NMR (400 MHz, CDCl₃), δ: 8.27 (s, 1H), 7.61 (d, J=9.2 Hz, 1H), 7.32 (dd, J=8.4, 2.4 Hz, 1H), 5.74 (dd, J=9.2, 2.4 Hz, 1H), 4.12-4.04 (m, 1H), 3.93-3.87 (m, 7H), 3.83-3.73 (m, 1H), 3.58-3.51 (m, 4H), 2.67-2.52 (m, 1H), 2.17 (s, 2H), 1.84-1.76 (m, 3H); LCMS (ESI) m/z calc. for C₂₂H₂₄F₃N₅O₄ 479.18; found, 480.6 [M+H]⁺.

### Synthesis of 3-iodo-5-isopropoxy-4-methyl-1-(tetrahydro-2H-pyran-2-yl)-1H-indazole (P89)

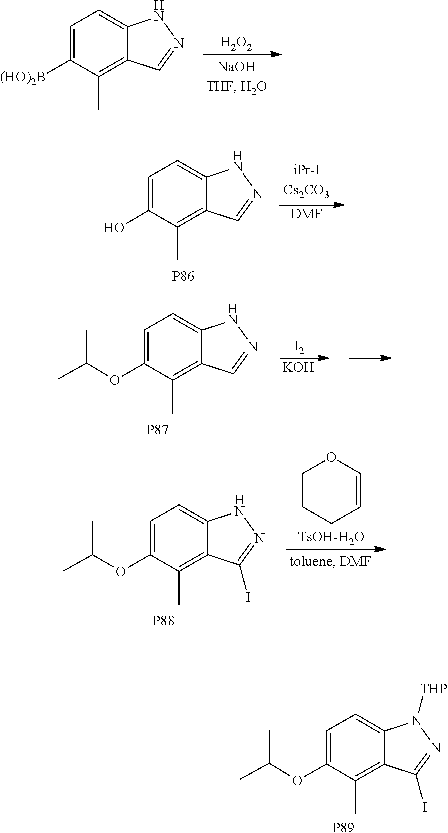

### Preparation 86. 4-Methyl-1H-indazol-5-ol (P86)

A solution of (4-methyl-1H-indazol-5-yl)boronic acid (7.0 g, 40 mmol), H₂O₂ (30% in H₂O, 5.4 g, 48 mmol), NaOH (2.0 g, 50 mmol) in H₂O (42 mL) in THF (210 mL) was stirred at rt for 3 h. The mixture was quenched with 1 N HCl_{(aq)} and extracted with EtOAc. The organic layers were dried over Na₂SO₄₍ₛ₎, filtered, and concentrated under reduced pressure to give P86 (3.8 g, 65%) as a yellow solid which was used in the next step without further purification. ¹H NMR (400 MHz, DMSO-d₆), δ: 12.68 (br. s, 1H), 8.72 (s, 1H), 7.90 (s, 1H), 7.14 (d, J=8.8 Hz, 1H), 6.91 (d, J=8.8 Hz, 1H), 2.31 (s, 3H); LRMS (ESI) m z 149.0 [M+H]⁺.

### Preparation 87. 5-Isopropoxy-4-methyl-1H-indazole (P87)

To a solution of P86 (4.3 g, 29 mmol) and cesium carbonate (19 g, 58 mmol) in N,N-dimethylformamide (50 mL) was added 2-iodopropane (7.4 g, 44 mmol) at rt. The mixture was stirred at rt for 2 h. The mixture was diluted with EtOAc and washed with water. The organic layer was dried over Na₂SO₄₍ₛ₎, filtered, and concentrated under reduced pressure. The crude was purified by silica gel column chromatography (0-50% EtOAc in n-hexane) to give P87 (3.0 g, 54%) as a yellow solid. ¹H NMR (400 MHz, CDCl₃), δ: 8.03 (s, 1H), 7.24 (d, J=8.8 Hz, 1H), 7.11 (d, J=8.8 Hz, 1H), 4.37 (septet, J=5.6 Hz, 1H), 2.48 (s, 3H), 1.33 (d, J=5.6 Hz, 6H); LRMS (ESI) m/z 191.3 [M+H]⁺.

### Preparation 88. 3-Iodo-5-isopropoxy-4-methyl-1H-indazole (P88)

To a solution of P87 (3.0 g, 16 mmol) and potassium hydroxide (3.5 g, 63 mmol) in N,N-dimethylformamide (15 mL) was added iodine (8.0 g, 32 mmol) at rt. The mixture was stirred at rt for 0.5 h. The mixture was treated with EtOAc and washed with Na₂S₂O_{3(aq)}. The organic layers were dried over Na₂SO₄₍ₛ₎, filtered, and concentrated under reduced pressure. The residue was treated with EtOAc/n-hexane (1:5). The resulting precipitate was collected by filtration to give P88 (2.8 g, 56%) as a yellow solid. ¹H NMR (400 MHz, CDCl₃): δ 11.35 (br s, 1H), 7.46-7.34 (m, 1H), 7.15 (d, J=9.0 Hz, 1H), 4.35 (septet, J=6.0 Hz, 1H), 2.74 (s, 3H), 1.33 (d, J=6.0 Hz, 6H); LRMS (ESI) m/z 317.1 [M+H]⁺.

### Preparation 89. 3-Iodo-5-isopropoxy-4-methyl-1-(tetrahydro-2H-pyran-2-yl)-1H-indazole (P89)

A solution of P88 (1.0 g, 3.2 mmol), p-toluenesulfonic acid monohydrate (0.30 g, 1.6 mmol), 3,4-dihydro-2H-pyran (0.53 g, 6.3 mmol) and N,N-dimethylformamide (1.0 mL) in toluene (15 mL) was stirred at 60° C. for 16 h. The mixture was treated with EtOAc and washed with NaHCO_{3(aq)}. The organic layers were dried over Na₂SO₄₍ₛ₎, filtered, and concentrated under reduced pressure. The crude was purified by silica gel column chromatography (0-50% EtOAc in n-hexane) to give P89 (0.9 g, 71%) as a yellow solid. ¹H NMR (400 MHz, CDCl₃), δ: 7.35 (d, J=9.0 Hz, 1H), 7.12 (d, J=9.0 Hz, 1H), 5.61 (dd, J=9.4, 2.8 Hz, 1H), 4.33 (m, 1H), 4.08-3.96 (septet, J=6.0 Hz, 1H), 3.77-3.65 (m, 1H), 2.71 (s, 3H), 2.60-2.47 (m, 1H), 2.22-2.08 (m, 1H), 2.08-1.97 (m, 1H), 1.80-1.70 (m, 2H), 1.66-1.60 (m, 1H), 1.31 (d, J=6.0 Hz, 3H), 1.30 (d, J=6.0 Hz, 3H); LRMS (ESI) m/z 401.1 [M+H]⁺.

### Synthesis of 3-iodo-5-isopropoxy-4-methyl-1-(tetrahydro-2H-pyran-2-yl)-1H-indazole (P90)

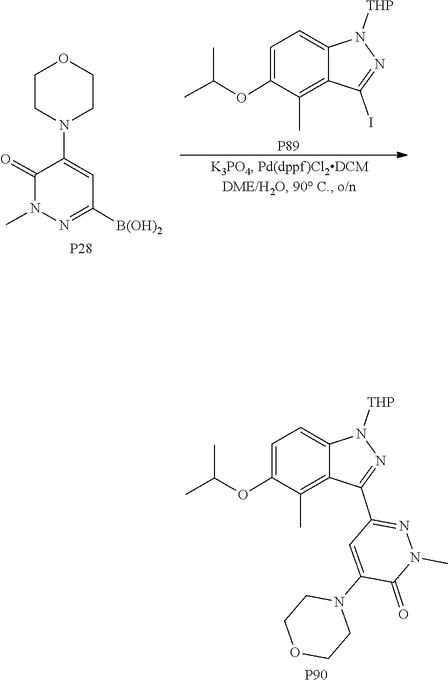

### Preparation 90. 6-(5-Isopropoxy-4-methyl-1-(tetrahydro-2H-pyran-2-yl)-1H-indazol-3-yl)-2-methyl-4-morpholinopyridazin-3(2H)-one (P90)

To a solution of P28 (crude, 1.74 mmol) in dimethoxyethane (15 mL) was added P89 (580 mg, 1.45 mmol) and potassium phosphate (2 M in water, 2.2 mL, 4.35 mmol). The mixture was degassed with argon for 15 min. The mixture was added [1,1′-Bis(diphenylphosphino)ferrocene]dichloropalladium(II) dichloromethane complex (118 mg, 0.15 mmol) and degassed with argon again. The mixture was stirred at 90° C. for overnight and then cooled to rt. The solution was filtered through a pad of celite and the filtrate was concentrated. The residue was purified by silica gel chromatography (0-100% EtOAc in n-hexane). The resulting solid was washed with methanol to give P90 (323 mg) as a yellow solid. ¹H NMR (400 MHz, CDCl₃), δ: 7.39 (d, J=9.0 Hz, 1H), 7.15 (d, J=9.0 Hz, 1H), 6.84 (s, 1H), 5.69 (dd, J=9.6, 2.4 Hz, 1H), 4.36 (septet, J=6.0 Hz, 1H), 3.89-3.85 (m, 7H), 3.78-3.71 (m, 1H), 3.53-3.50 (m, 4H), 2.61-2.51 (m, 1H), 2.42 (s, 3H), 2.16-2.11 (m, 1H), 2.07-2.06 (m, 1H), 1.78-1.72 (m, 2H), 1.65-1.64 (m, 2H), 1.32 (d, J=6.0 Hz, 6H).

### Synthesis of 2-methyl-4-morpholino-6-(3-(tetrahydro-2H-pyran-2-yl)-3,7,8,9-tetrahydropyrano[3,2-e]indazol-1-yl)pyridazin-3(2H)-one (P96)

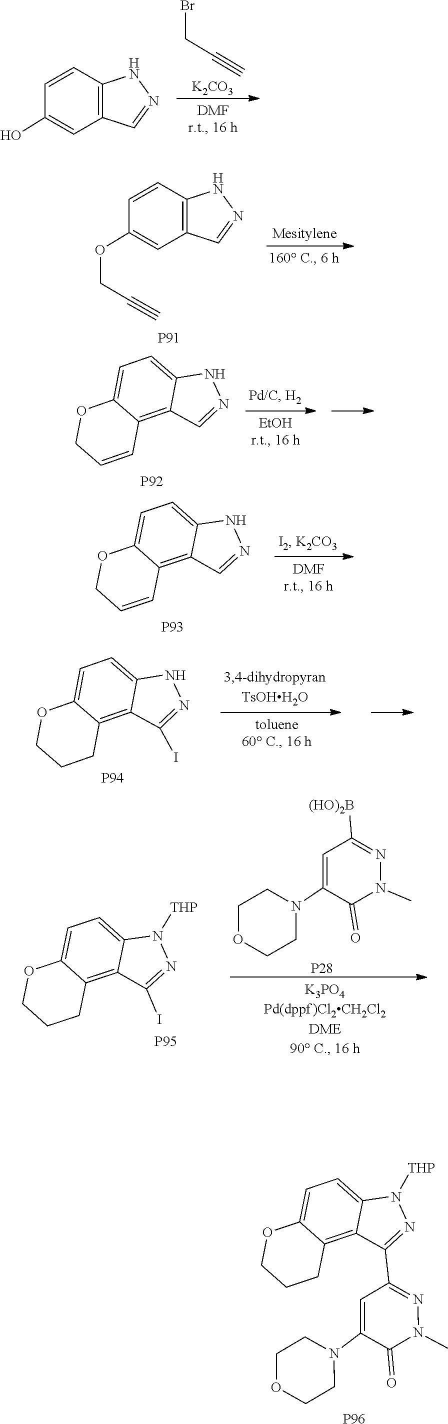

### Preparation 91. 5-(Prop-2-yn-1-yloxy)-1H-indazole (P91)

To a solution of 1H-indazol-5-ol (1.00 g, 7.45 mmol, 1.0 eq) in DMF (8.0 mL) was added potassium carbonate (2.06 g, 14.9 mmol, 2.0 eq) and 80% of 3-bromoprop-1-yne in toluene solution (1.00 mL, 8.94 mmol, 1.2 eq) at rt. After the reaction was stirred at rt for 16 h, the mixture was poured into ice water and extracted with EtOAc. The organic layers were dried over MgSO₄₍ₛ₎, filtered, and concentrated under reduced pressure. The residue was purified by silica gel column chromatography (50% EtOAc in n-hexane) to afford P91 (682 mg, 53%) as an orange solid. ¹H NMR (400 MHz, DMSO-d₆), δ: 12.93 (br. s, 1H), 7.96 (s, 1H), 7.45 (d, J=9.2 Hz, 1H), 7.25 (d, J=2.4 Hz, 1H), 7.02 (dd, J=9.2, 2.4 Hz, 1H), 4.78 (d, J=2.4 Hz, 2H), 3.54 (t, J=2.4 Hz, 1H).

### Preparation 92. 3,7-Dihydropyrano[3,2-e]indazole (P92)

A solution of P91 (100 mg, 0.581 mmol, 1.0 eq) in mesitylene (1.5 mL) in a sealed tube was heated to 160° C. for 6 h. After the reaction mixture was cooled to rt, the solution was diluted with water and extracted with EtOAc. The organic layers were dried over MgSO₄₍ₛ₎, filtered, and purified by silica gel column chromatography (25% EtOAc in n-hexane) to afford P92 (50.0 mg, 50%) as a yellow solid. ¹H NMR (400 MHz, CDCl₃), δ: 10.00 (br. s, 1H), 8.03 (s, 1H), 7.24 (d, J=8.8 Hz, 1H), 6.95 (d, J=8.8 Hz, 1H), 6.81 (d, J=9.6 Hz, 1H), 5.94-5.89 (m, J=9.6, 4.0 Hz, 1H), 4.86-4.84 (m, 2H).

### Preparation 93. 3,7,8,9-Tetrahydropyrano[3,2-e]indazole (P93)

To a solution of Pd/C (24.4 mg, 10% w/w) in ethanol (15 mL) was added a solution of P92 (244 mg, 1.42 mmol, 1.0 eq) in ethanol (5.0 mL). After the solution was stirred at rt for 16 h under the hydrogen atmosphere, the reaction solution was filtered through a pad of Celite and the filtrate was concentrated under reduced pressure to afford P93 (224 mg, 91%) as a grey solid which was used in next step without purification. ¹H NMR (400 MHz, CDCl₃), δ: 9.99 (br. s, 1H), 7.95 (s, 1H), 7.22 (d, J=8.8 Hz, 1H), 6.93 (d, J=8.8 Hz, 1H), 4.24-4.22 (m, 2H), 3.00 (t, J=6.8 Hz, 2H), 2.15-2.09 (m, 2H).

### Preparation 94. 1-Iodo-3,7,8,9-tetrahydropyrano[3,2-e]indazole (P94)

To a solution of P93 (41.0 mg, 0.235 mmol, 1.0 eq) in DMF (2.0 mL) was added potassium carbonate (97.5 mg, 0.706 mmol, 3.0 eq) and iodine (119 mg, 0.471 mmol, 2.0 eq) at rt. After the solution was stirred at rt for 16 h, the solution was diluted with water. The mixture solution was extracted with EtOAc and washed with Na₂S₂O_{3(aq)}. The organic layer was dried over MgSO₄₍ₛ₎, filtered, and concentrated under reduced pressure to afford crude P94 (59 mg, 83%) as a brown oil. ¹H NMR (400 MHz, CDCl₃), δ: 10.44 (br. s, 1H), 7.23 (d, J=8.8 Hz, 1H), 6.95 (d, J=8.8 Hz, 1H), 4.20-4.18 (m, 2H), 3.40 (t, J=6.8 Hz, 2H), 2.12-2.06 (m, 2H).

### Preparation 95. 1-Iodo-3-(tetrahydro-2H-pyran-2-yl)-3,7,8,9-tetrahydropyrano[3,2-e]indazole (P95)

To a solution of P94 (59.0 mg, 0.196 mmol, 1.0 eq) in toluene (3.5 mL) was added 3,4-dihydropyran (35.8 μg, 0.393 mmol, 2.0 eq) and p-toluenesulfonic acid monohydrate (7.48 mg, 0.0393 mmol, 0.2 eq) at rt. After the reaction solution was stirred at 60° C. for 16 h, the reaction was diluted with water and extracted with EtOAc. The organic layers were dried over MgSO₄₍ₛ₎, filtered, and concentrated under reduced pressure. The residue was purified by silica gel column chromatography (33% EtOAc in n-hexane) to afford P95 (47 mg, 62%) as a grey oil. ¹H NMR (400 MHz, CDCl₃), δ: 7.32 (d, J=8.8 Hz, 1H), 6.93 (d, J=8.8 Hz, 1H), 5.59 (dd, J=9.6, 2.8 Hz, 1H), 4.19-4.16 (m, 2H), 4.02-3.97 (m, 1H), 3.72-3.66 (m, 1H), 3.89 (t, J=6.8 Hz, 2H), 2.55-2.45 (m, 1H), 2.15-1.98 (m, 4H), 1.75-1.72 (m, 2H), 1.65-1.61 (m, 1H).

### Preparation 96. 2-Methyl-4-morpholino-6-(3-(tetrahydro-2H-pyran-2-yl)-3,7,8,9-tetrahydropyrano[3,2-e]indazol-1-yl)pyridazin-3(2H)-one (P96)

To a crude solution of P28 was added P95 (47.0 mg, 0.122 mmol, 1.0 eq), 2 M K₃PO_{4(aq)} (0.183 mL) and 1,2-dimethoxyethane (4.5 mL) at rt. The mixture solution was degassed with argon_{(g)} and then added [1,1′-bis(diphenylphosphino)ferrocene]dichloropalladium(II) dichloromethane complex (9.99 mg, 0.0122 mmol, 0.1 eq). After the reaction solution was stirred at 90° C. for 16 h, the solution was filtered through a pad of Celite and washed the pad with EtOAc. The mixture solution was washed with water. The organic layer was dried over MgSO₄₍ₛ₎, filtered, and concentrated under reduced pressure. The residue was purified by silica gel column chromatography (33% EtOAc in n-hexane) to afford P96 (35 mg, 63%) as a white solid. ¹H NMR (400 MHz, CDCl₃), δ: 7.36 (d, J=9.2 Hz, 1H), 6.97 (d, J=9.2 Hz, 1H), 6.88 (s, 1H), 5.67 (dd, J=9.6, 2.8 Hz, 1H), 4.22-4.19 (m, 2H), 3.89-3.84 (m, 8H), 3.54-3.51 (m, 4H), 3.46-3.44 (m, 2H), 2.97 (t, J=6.8 Hz, 2H), 2.56-2.49 (m, 1H), 2.17-2.09 (m, 1H), 2.04-1.98 (m, 3H), 1.78-1.72 (m, 2H).

### Synthesis of 2-methyl-4-morpholino-6-(1-(tetrahydro-2H-pyran-2-yl)-1,6,7,8-tetrahydropyrano[2,3-f]indazol-3-yl)pyridazin-3(2H)-one (P106)

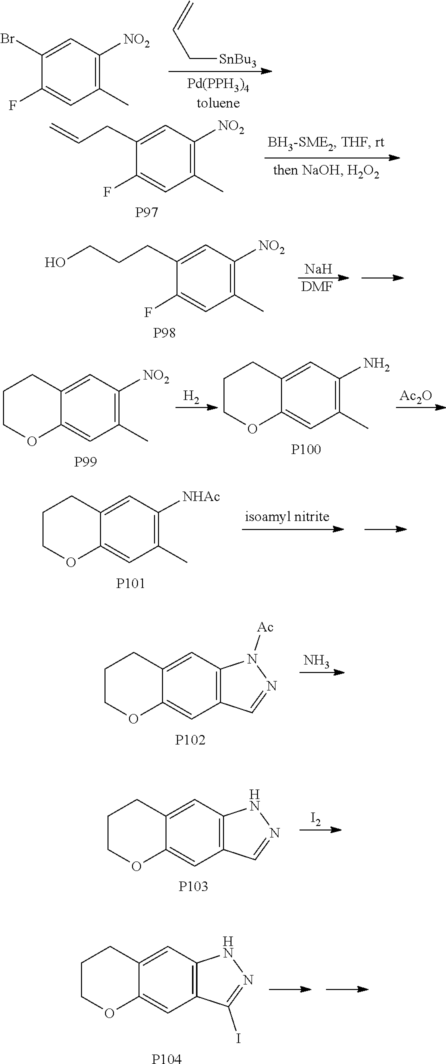

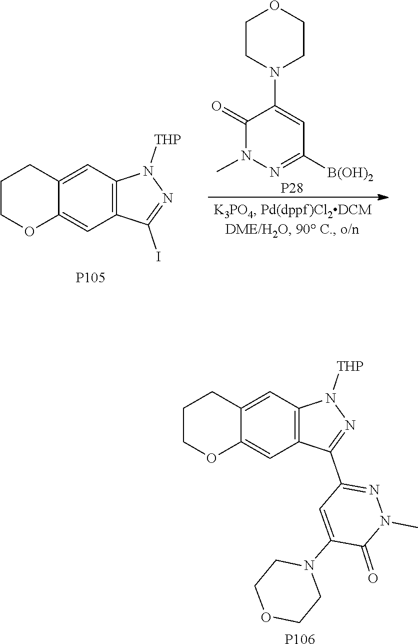

### Preparation 97. 1-Allyl-2-fluoro-4-methyl-5-nitrobenzene (P97)

A solution of 1-bromo-2-fluoro-4-methyl-5-nitro-benzene (5.0 g, 21 mmol), allyltributylstannane (7.8 g, 23 mmol) and tetrakis(triphenylphosphine)palladium (0) (1.2 g, 1.0 mmol) in toluene (50 mL) was degassed with argon. The mixture was heated at 110° C. and stirred for overnight. KF_{(aq)} was added at rt and filtrated with celite. The filtrate was extracted with EtOAc, dried over Na₂SO₄₍ₛ₎, filtered, and concentrated. The residue was purified by silica gel chromatography (0-30% EtOAc in n-hexane) to give P97 (4.1 g, 100%). ¹H NMR (400 MHz, CDCl₃), δ: 7.92 (d, J=7.0 Hz, 1H), 6.99 (d, J=9.8 Hz, 1H), 6.04-5.83 (m, 1H), 5.23-5.03 (m, 2H), 3.41 (d, J=6.6 Hz, 2H), 2.58 (s, 3H).

### Preparation 98. 3-(2-fluoro-4-methyl-5-nitrophenyl)propan-1-ol (P98)

A solution of P98 (4.1 g, 21 mmol) and 2M dimethyl sulfide borane (21 mL, 42 mmol) in THF (50 mL) was stirred at rt for 4 h. 1N NaOH_{(aq)} (150 mL) and 30% H₂O₂ (75 mL) was added to the reaction and it was stirred at rt for 1 h. The mixture was extracted with EtOAc. The combined organic layer was dried over Na₂SO₄₍ₛ₎, filtered, and concentrated. The residue was purified by silica gel chromatography (0-30% EtOAc in n-hexane) to give P99 (1.6 g, 36%). ¹H NMR (400 MHz, CDCl₃), δ: 7.94 (dd, J=7.0, 1.8 Hz, 1H), 6.98 (dd, J=10.0, 1.2 Hz, 1H), 3.69 (t, J=6.2 Hz, 2H), 2.77 (t, J=7.8 Hz, 2H), 2.58 (d, J=2.4 Hz, 3H), 1.95-1.82 (m, 2H).

### Preparation 99. 7-Methyl-6-nitrochromane (P99)

To a solution of P98 (1.4 g, 6.6 mmol) in DMF (140 mL) was added NaH (1.1 g, 28 mmol) and the reaction was stirred at 70° C. for overnight. The mixture was treated with water. The mixture was extracted with EtOAc, dried over Na₂SO₄₍ₛ₎, filtered, and concentrated. The residue was purified by silica gel chromatography (0-10% EtOAc in n-hexane) to give P99 (568 mg, 45%). ¹H NMR (400 MHz, CDCl₃), δ: 7.88 (s, 1H), 6.67 (s, 1H), 4.29-4.17 (m, 2H), 2.80 (t, J=6.4 Hz, 2H), 2.56 (s, 3H), 2.07-1.98 (m, 2H).

### Preparation 100. 7-Methylchroman-6-amine (P100)

A solution of P99 (0.69 g, 3.6 mmol) and Pd/C (200 mg) in MeOH (20 mL) was added stirred at rt under H_{2(g)} (1 atm) for 5 h. The mixture was filtered through a pad of celite. The filtrate was concentrated in vacuo to give P100 (580 mg, 99%). ¹H NMR (400 MHz, CDCl₃), δ: 6.53 (s, 1H), 6.38 (s, 1H), 4.13-4.04 (m, 2H), 3.29 (br. s, 2H), 2.69 (t, J=6.6 Hz, 2H), 2.11 (s, 3H), 2.00-1.90 (m, 2H).

### Preparation 101. N-(7-methylchroman-6-yl)acetamide (P101)

A solution of P100 (0.58 mg, 3.6 mmol), Ac₂O (0.50 mL, 5.4 mmol) and Et₃N (1.0 mL, 6.9 mmol) in DCM (10.0 mL) was stirred at rt for overnight. The mixture was washed with NaHCO_{3(aq)}, dried over Na₂SO₄₍ₛ₎, filtered, and concentrated to give P101 (630 mg, 85%). ¹H NMR (400 MHz, CDCl₃), δ: 7.25 (s, 1H), 6.77 (s, 1H), 6.62 (s, 1H), 4.20-4.07 (m, 2H), 2.74 (t, J=6.6 Hz, 2H), 2.17 (d, J=2.6 Hz, 6H), 2.03-1.91 (m, 2H).

### Preparation 102. 1-(7,8-Dihydropyrano[2,3-f]indazol-1(6H)-yl)ethan-1-one (P102)

A solution of P101 (0.63 g, 3.1 mmol), Ac₂O (1.3 mL, 14 mmol), KOAc (0.45 g, 4.6 mmol) and isoamyl nitrite (1.4 g, 12 mmol) in toluene (15 mL) was 80° C. for 16 h. The mixture was filtered through celite and washed with EtOAc. The filtrate was washed with water, dried over Na₂SO₄₍ₛ₎, filtered, and concentrated. The residue was purified by silica gel chromatography (0-100% EtOAc in n-hexane) to give P102 (540 mg, 81%). ¹H NMR (400 MHz, CDCl₃), δ: 8.14 (d, J=0.6 Hz, 1H), 7.98 (d, J=0.8 Hz, 1H), 7.08-7.04 (m, 1H), 4.27-4.18 (m, 2H), 3.01 (t, J=6.6 Hz, 2H), 2.75 (s, 3H), 2.08-2.03 (m, 2H).

### Preparation 103. 1,6,7,8-tetrahydropyrano[2,3-f]indazole (P103)

A solution of P102 (0.54 mg, 2.5 mmol) and NH_{3(MeOH)} (1.8 mL, 7M in MeOH) in MeOH/CH₂Cl₂ (17 mL, 16:1 (v/v)) was stirred at rt for 2 h. The mixture was concentrated to give P103 (0.42 mg, 97%) which was used in the next step without purification. LCMS (ESI) m/z 174.8 [M+H]⁺.

### Preparation 104. 3-Iodo-1,6,7,8-tetrahydropyrano[2,3-f]indazole (P104)

To a solution of P103 (0.42 mg, 2.4 mmol) and potassium hydroxide (540 mg, 9.64 mmol) in N,N-dimethylformamide (10.0 mL) was added iodine (1.2 g, 4.7 mmol) at rt. The mixture was stirred at rt for 1 h and treated with EtOAc. The solution was washed with Na₂S₂O_{3(aq)}. The organic layer was dried over Na₂SO₄₍ₛ₎, filtered, and concentrated under reduced pressure to give P104 (720 mg). ¹H NMR (400 MHz, CDCl₃), δ: 11.06 (br. s, 1H), 7.17 (s, 1H), 6.83 (s, 1H), 4.25-4.15 (m, 2H), 3.00-2.95 (m, 2H), 2.10-2.01 (m, 2H).

### Preparation 105. 3-Iodo-1-(tetrahydro-2H-pyran-2-yl)-1,6,7,8-tetrahydropyrano[2,3-f]indazole (P105)

A solution of P104 (720 mg, 2.40 mmol), p-toluenesulfonic acid monohydrate (228 mg, 1.20 mmol), 3,4-dihydro-2H-pyran (404 mg, 4.80 mmol) and N,N-dimethylformamide (0.7 mL) in toluene (12 mL) was stirred at 60° C. for 16 h. The mixture was treated with EtOAc and washed with NaHCO_{3(aq)}. The organic layer was dried over Na₂SO₄₍ₛ₎, filtered, and concentrated under reduced pressure. The crude was purified by silica gel column chromatography (0-50% EtOAc in n-hexane) to give P105 (0.4 mg, 43%). ¹H NMR (400 MHz, CDCl₃), δ: 7.23 (s, 1H), 6.82 (s, 1H), 5.60 (dd, J=9.2, 2.8 Hz, 1H), 4.25-4.17 (m, 2H), 4.05-3.93 (m, 1H), 3.77-3.65 (m, 1H), 3.00 (t, J=6.4 Hz, 2H), 2.61-2.41 (m, 1H), 2.23-2.09 (m, 1H), 2.07-2.01 (m, 3H), 1.81-1.71 (m, 2H).

### Preparation 106. 2-Methyl-4-morpholino-6-(1-(tetrahydro-2H-pyran-2-yl)-1,6,7,8-tetrahydropyrano[2,3-f]indazol-3-yl)pyridazin-3(2H)-one (P106)

To a solution of P28 (crude, 1.00 mmol) in dimethoxyethane (8.0 mL) was added P105 (320 mg, 0.83 mmol) and potassium phosphate (2 M in water, 1.2 mL, 2.49 mmol). The mixture was degassed by argon for 15 min. The mixture was added [1,1′-Bis(diphenylphosphino)ferrocene]dichloropalladium(II), complex with dichloromethane (68 mg, 0.08 mmol) and degassed by argon. The mixture was stirred at 90° C. for overnight and cooled to rt. The solution was filtered through celite. The filtrate was concentrated in vacuo and then purified by silica gel chromatography (0-100% EtOAc in n-hexane). The resulting solid was washed with methanol to give P106 (128 mg) as an orange solid. ¹H NMR (400 MHz, CDCl₃), δ: 7.76 (s, 1H), 7.27 (s, 1H), 7.26 (s, 1H), 5.66 (dd, J=9.7, 2.6 Hz, 1H), 4.25-4.22 (m, 2H), 4.11-4.07 (m, 1H), 3.90-3.80 (m, 7H), 3.79-3.73 (m, 1H), 3.53-3.51 (m, 4H), 3.01 (t, J=6.6 Hz, 2H), 2.67-2.57 (m, 1H), 2.17-2.16 (m, 1H), 2.09-2.03 (m, 2H), 1.81-1.75 (m, 2H), 1.67-1.63 (m, 2H).

### Synthesis of 6-(5-(difluoromethoxy)-1-(tetrahydro-2H-pyran-2-yl)-1H-indazol-3-yl)-2-methyl-4-morpholinopyridazin-3(2H)-one (P112)

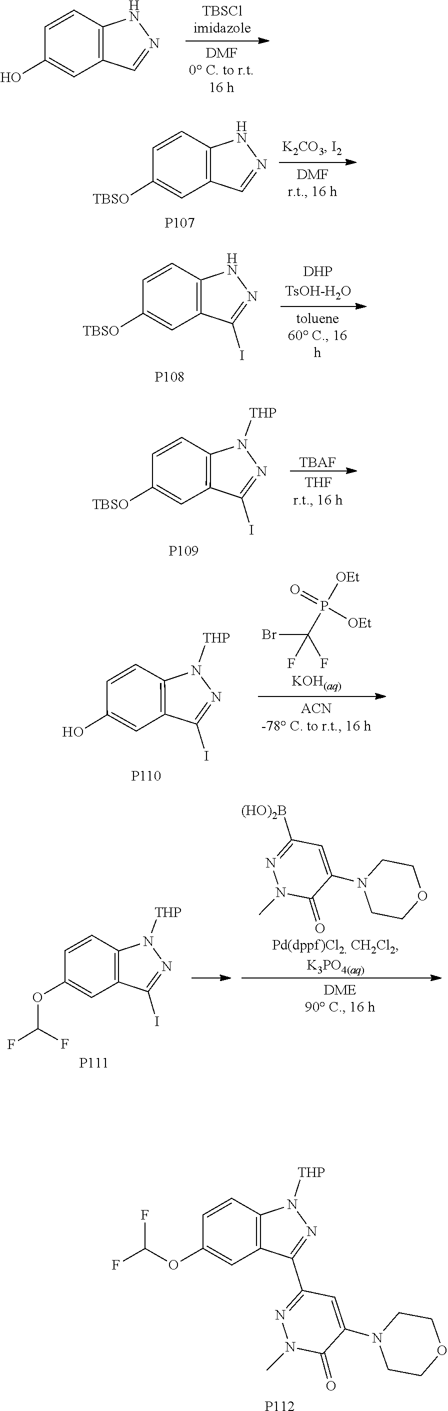

### Preparation 107. 5-((tert-Butyldimethylsilyl)oxy)-1H-indazole (P107)

To a solution of 1H-indazol-5-ol (5.00 g, 37.28 mmol, 1.0 eq) and imidazole (5.08 g, 74.55 mmol, 2.0 eq) in DMF (50 mL) was added tert-butyldimethylsilyl chloride (TBSCl, 8.43 g, 55.91 mmol, 1.5 eq) at 0° C. The reaction mixture was stirred at rt for 16 h. The solution was treated with water and extracted with ethyl acetate three times. The combined organic layers were washed with brine, dried over Na₂SO₄₍ₛ₎, filtered, and concentrated in vacuo to give the crude product P107 (8.95 g, 97%) as a white solid which was used in the next step without further purification. ¹H NMR (400 MHz, CDCl₃), δ: 9.93 (br. s, 1H), 7.95 (s, 1H), 7.35 (d, J=9.0 Hz, 1H), 7.12 (d, J=2.2 Hz, 1H), 6.98 (dd, J=9.0, 2.2 Hz, 1H), 1.01 (s, 9H), 0.21 (s, 6H).

### Preparation 108. 5-((tert-Butyldimethylsilyl)oxy)-3-iodo-1H-indazole (P108)

To a solution of P107 (8.95 g, 36.0 mmol, 1.0 eq) in DMF (125 mL) was added potassium carbonate (9.96 g, 72.06 mmol, 2.0 eq), followed by iodine (13.72 g, 54.05 mmol, 1.5 eq). The reaction was stirred at rt for 16 h. The mixture was quenched with saturated Na₂S₂O_{3(aq)} and extracted with ethyl acetate three times. The combined organic layers were dried over Na₂SO₄₍ₛ₎, filtered, and concentrated in vacuo to give a residue, which was purified by silica gel column chromatography (n-hexane:EtOAc=3:1) to give P108 (6.83 g, 51%) as a yellow solid. ¹H NMR (400 MHz, CDCl₃), δ: 10.28 (br. s, 1H), 7.33 (d, J=9.0 Hz, 1H), 7.03 (dd, J=9.0, 2.2 Hz, 1H), 6.85 (d, J=2.2 Hz, 1H), 1.01 (s, 9H), 0.23 (s, 6H).

### Preparation 109. 5-((tert-Butyldimethylsilyl)oxy)-3-iodo-1-(tetrahydro-2H-pyran-2-yl)-1H-indazole (P109)

To a solution of P108 (6.83 g, 18.25 mmol, 1.0 eq) in toluene (70 mL) was added 3,4-dihydro-2H-pyran (DHP, 3.33 mL, 36.50 mmol, 2.0 eq) and p-toluenesulfonic acid monohydrate (694.2 mg, 3.65 mmol, 0.2 eq) at 0° C. The reaction mixture was stirred at 60° C. for 16 h. The solution was concentrated, treated with water, and extracted with ethyl acetate three times. The combined organic layers were dried over Na₂SO₄₍ₛ₎, filtered, and concentrated in vacuo to give a residue, which was purified by silica gel column chromatography (n-hexane:EtOAc=20:1 to 2:1) to give P109 (5.89 g, 70% yield) as a yellow solid. ¹H NMR (400 MHz, CDCl₃), δ: 7.42 (d, J=9.2 Hz, 1H), 7.01 (dd, J=9.2, 2.2 Hz, 1H), 6.81 (d, J=2.2 Hz, 1H), 5.63 (dd, J=9.6, 2.8 Hz, 1H), 4.06-3.99 (m, 1H), 3.76-3.68 (m, 1H), 2.59-2.47 (m, 1H), 2.18-2.10 (m, 1H), 2.09-2.02 (m, 1H), 1.78-1.62 (m, 3H), 1.01 (s, 9H), 0.21 (s, 6H).

### Preparation 110. 3-Iodo-1-(tetrahydro-2H-pyran-2-yl)-1H-indazol-5-ol (P110)

To a solution of P109 (5.89 g, 12.85 mmol, 1.0 eq) in THF (58 mL) was added 1.0 M tetra-n-butylammonium fluoride solution (TBAF, 64.2 mL, 64.2 mmol, 5.0 eq), and the resulting mixture was stirred at rt for 16 h. The solution was concentrated, treated with water and extracted with ethyl acetate three times. The combined organic layers were washed with brine, dried over Na₂SO₄₍ₛ₎, filtered, and concentrated in vacuo to give the residue. The residue was purified by silica gel column chromatography (n-hexane:EtOAc=2:1 to 1:1) to give P110 (3.84 g, 87% yield) as a light-yellow solid. ¹H NMR (400 MHz, DMSO-d₆), δ: 9.51 (s, 1H), 7.57 (d, J=9.0 Hz, 1H), 7.02 (dd, J=9.0, 2.2 Hz, 1H), 6.64 (dd, J=2.2 Hz, 1H), 5.74 (dd, J=10.0, 2.4 Hz, 1H), 3.89-3.82 (m, 1H), 3.74-3.65 (m, 1H), 2.38-2.26 (m, 1H), 2.04-1.90 (m, 2H), 1.78-1.65 (m, 1H), 1.60-1.51 (m, 2H).

### Preparation 111. 5-(Difluoromethoxy)-3-iodo-1-(tetrahydro-2H-pyran-2-yl)-1H-indazole (P111)

To a solution of P110 (500 mg, 1.45 mmol, 1.0 eq) in ACN (1.9 mL) was added 5.3 M potassium hydroxide aqueous solution (1.92 mL, 10.17 mmol, 7.0 eq), followed by diethyl (bromodifluoromethyl)phosphonate (0.28 mL, 1.60 mmol, 1.1 eq) at −78° C. dropwise. The reaction mixture was stirred at rt for 2 h. The solution was treated with water and extracted with ethyl acetate three times. The combined organic layers were dried over Na₂SO₄₍ₛ₎, filtered, and concentrated in vacuo to give the residue. The residue was purified by silica gel column chromatography (n-hexane:EtOAc=5:1) to give P111 (430 mg, 75% yield) as a white solid. ¹H NMR (400 MHz, CDCl₃), δ: 7.57 (d, J=9.2 Hz, 1H), 7.28-7.25 (m, 1H), 7.21-7.19 (m, 1H), 6.54 (t, J=74.0 Hz, 1H), 5.69 (dd, J=9.2, 2.8 Hz, 1H), 4.04-3.97 (m, 1H), 3.77-3.69 (m, 1H), 2.58-2.46 (m, 1H), 2.20-2.03 (m, 2H), 1.82-1.61 (m, 3H).

### Preparation 112. 6-(5-(Difluoromethoxy)-1-(tetrahydro-2H-pyran-2-yl)-1H-indazol-3-yl)-2-methyl-4-morpholinopyridazin-3(2H)-one (P112)

A solution of P27 (150.0 mg, 0.653 mmol, 1.0 eq), bis(pinacolato)diboron (829.3 mg, 3.27 mmol, 5.0 eq) and potassium acetate (128.2 mg, 1.31 mmol, 2.0 eq) in 1,4-dioxane (5 mL) was degassed by argon for 10 min. To the reaction mixture was added dicyclohexyl[2′,4′,6′-tris(propan-2-yl)[1,1′-biphenyl]-2-yl]phosphane (XPhos, 62.3 mg, 0.131 mmol, 0.2 eq) and [1,1′-bis(diphenylphosphino)ferrocene]dichloropalladium(II) (47.8 mg, 0.065 mmol, 0.1 eq). The reaction was stirred at 50° C. for 16 h to give P28. To the above mixture P28 was added P111 (200 mg, 0.507 mmol, 1.0 eq), 2.0 M potassium phosphate aqueous solution (0.76 mL, 1.52 mmol, 3.0 eq) and DME (5.0 mL). The reaction mixture was degassed by argon for 10 min. To the reaction mixture was added [1,1′-bis(diphenylphosphino)ferrocene]dichloropalladium(II) dichloromethane complex (41.4 mg, 0.051 mmol, 0.1 eq). After the reaction was stirred at 90° C. for 16 h, the mixture was treated with water and extracted with ethyl acetate three times. The combined organic layers were dried over Na₂SO₄₍ₛ₎, filtered, and concentrated in vacuo to give a residue, which was purified by silica gel column chromatography (n-hexane:EtOAc=3:1), followed by slurry (with methanol) to give P112 (104 mg, 44%) as a light-yellow solid. ¹H NMR (400 MHz, CDCl₃), δ: 8.16 (d, J=1.6 Hz, 1H), 7.59 (d, J=9.2 Hz, 1H), 7.29-7.24 (m, 2H), 6.56 (t, J=74.4 Hz, 1H), 5.73 (dd, J=9.6, 2.8 Hz, 1H), 4.12-4.05 (m, 1H), 3.93-3.88 (m, 7H), 3.82-3.73 (m, 1H), 3.59-3.52 (m, 4H), 2.67-2.55 (m, 1H), 2.23-2.13 (m, 1H), 2.12-2.05 (m, 1H), 1.85-1.74 (m, 2H), 1.73-1.65 (m, 1H).

### Synthesis of 2-methyl-6-(5-(methylsulfonyl)-1-(tetrahydro-2H-pyran-2-yl)-1H-indazol-3-yl)-4-morpholinopyridazin-3(2H)-one (P120)

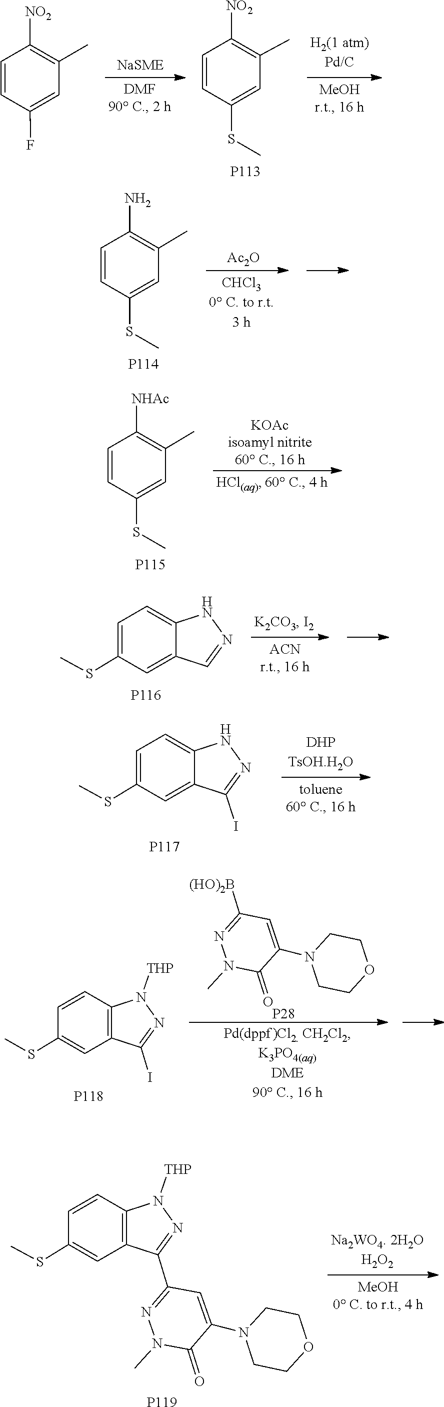

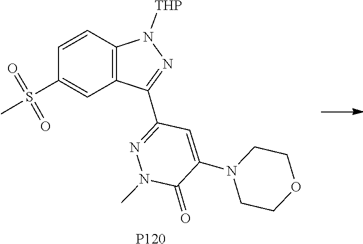

### Preparation 113. Methyl(3-methyl-4-nitrophenyl)sulfane (P113)

To a solution of 4-fluoro-2-methyl-1-nitro-benzene (1.10 g, 7.09 mmol, 1.0 eq) in DMF (12 mL) was added 15% sodium methanethiolate aqueous solution (3.48 g, 7.45 mmol, 1.05 eq). The reaction mixture was stirred at 90° C. for 2 h. The reaction mixture was treated with water and extracted with ethyl acetate three times. The combined organic layers were dried over Na₂SO₄₍ₛ₎, filtered, and concentrated in vacuo to give a crude product P113 (1.29 g, 99% yield, crude) as a yellow oil which was used in next step without further purification. ¹H NMR (400 MHz, CDCl₃), δ: 7.99 (d, J=8.4 Hz, 1H), 7.15-7.08 (m, 2H), 2.62 (s, 3H), 2.53 (s, 3H).

### Preparation 114. 2-Methyl-4-(methylthio)aniline (P114)

To a solution of P113 (1.29 g, 7.04 mmol, 1.0 eq) in methanol (70 mL) was added Pd/C powder (260 mg, 10% wt). The reaction was stirred at room temperature for 16 h under hydrogen balloon (1 atm). The solution was filtered through a pad of celite. The filtrate was concentrated in vacuo to give crude product P114 (1.07 g, 99% yield, crude) as a yellow oil. ¹H NMR (400 MHz, DMSO-d₆), δ: 6.95 (d, J=2.4 Hz, 1H), 6.91 (dd, J=8.4, 2.4 Hz, 1H), 6.56 (d, J=8.4 Hz, 1H), 4.88 (br. s, 2H, NH₂), 2.31 (s, 3H), 2.02 (s, 3H).

### Preparation 115/116. 2-Methyl-4-(methylthio)aniline (P116)

To a solution of P114 (1.07 g, 6.98 mmol, 1.0 eq) in chloroform (8.2 mL) was added acetic anhydride (1.50 mL, 15.85 mmol, 2.27 eq) dropwise at 0° C., and the resulting mixture was stirred at rt for 3 h. To the above reaction mixture was added potassium acetate (205.6 mg, 2.09 mmol, 0.3 eq) and isoamyl nitrite (2.02 mL, 15.01 mmol, 2.15 eq) at 60° C. After the solution was stirred at 60° C. for 16 h. The mixture was concentrated and 6.0 M HCl aq. solution (8.2 mL) was added to the mixture. The reaction was stirred at 60° C. for another 4 h. The pH value was adjusted to 10 by addition of NaOH_{(aq)} and the solution was extracted with ethyl acetate three times. The combined organic layers were dried over Na₂SO₄₍ₛ₎, filtered, and concentrated in vacuo to give the residue. The residue was purified by silica gel column chromatography (n-hexane:EtOAc=3:1) to give P116 (864 mg, 75% yield) as a yellow solid. ¹H NMR (400 MHz, CDCl₃), δ: 8.02 (d, J=1.2 Hz, 1H), 7.68 (dd, J=2.0, 1.2 Hz, 1H), 7.45-7.37 (m, 2H), 2.53 (s, 3H).

### Preparation 117. 3-Iodo-5-(methylthio)-1H-indazole (P117)

To a solution of P116 (430.0 mg, 2.62 mmol, 1.0 eq) in ACN (11 mL) was added potassium carbonate (723.7 mg, 5.24 mmol, 2.0 eq) followed by iodine (996.8 mg, 3.93 mmol, 1.5 eq). The reaction was stirred at rt for 16 h. The mixture was quenched with saturated Na₂S₂O_{3(aq)} and extracted with ethyl acetate three times. The combined organic layers were dried over Na₂SO₄₍ₛ₎, filtered, and concentrated in vacuo to give a residue, which was purified by silica gel column chromatography (n-hexane:EtOAc=4:1) to give P117 (718 mg, 95% yield) as a light-yellow solid. ¹H NMR (400 MHz, CDCl₃): δ 10.31 (br. s, 1H), 7.45-7.36 (m, 3H), 2.56 (s, 3H).

### Preparation 118. 3-Iodo-5-(methylthio)-1-(tetrahydro-2H-pyran-2-yl)-1H-indazole (P118)

To a solution of P117 (718.0 mg, 2.47 mmol, 1.0 eq) in toluene (10 mL) was added 3,4-dihydro-2H-pyran (DHP, 0.45 mL, 4.95 mmol, 2.0 eq) and p-toluenesulfonic acid monohydrate (94.2 mg, 0.50 mmol, 0.2 eq) at 0° C. The reaction mixture was stirred at 60° C. for 16 h. The solution was concentrated, treated with water, and extracted with ethyl acetate three times. The combined organic layers were dried over Na₂SO₄₍ₛ₎, filtered, and concentrated in vacuo to give a residue, which was purified by silica gel column chromatography (n-hexane:EtOAc=5:1) to give P119 (820 mg, 89% yield) as an orange solid. ¹H NMR (400 MHz, CDCl₃), δ: 7.52-7.31 (m, 3H), 5.66 (dd, J=9.2, 2.8 Hz, 1H), 4.04-3.98 (m, 1H), 3.76-3.67 (m, 1H), 2.59-2.47 (m, 4H), 2.19-2.10 (m, 1H), 2.09-2.02 (m, 1H), 1.81-1.61 (m, 3H).

### Preparation 119. 2-Methyl-6-(5-(methylthio)-1-(tetrahydro-2H-pyran-2-yl)-1H-indazol-3-yl)-4-morpholinopyridazin-3(2H)-one (P119)

A solution of P27 (150.0 mg, 0.653 mmol, 1.0 eq), bis(pinacolato)diboron (829.3 mg, 3.27 mmol, 5.0 eq) and potassium acetate (128.2 mg, 1.31 mmol, 2.0 eq) in 1,4-dioxane (5 mL) was degassed by argon for 10 min. To the reaction mixture was added dicyclohexyl[2′,4′,6′-tris(propan-2-yl)[1,1′-biphenyl]-2-yl]phosphane (XPhos, 62.3 mg, 0.131 mmol, 0.2 eq) and [1,1′-bis(diphenylphosphino)ferrocene]dichloropalladium(II) (Pd(dppf)Cl₂, 47.8 mg, 0.065 mmol, 0.1 eq). The reaction was stirred at 50° C. for 16 h to give P28. To the above mixture P28 was added P118 (200.0 mg, 0.534 mmol, 1.0 eq), 2.0 M potassium phosphate aqueous solution (0.8 mL, 1.60 mmol, 3.0 eq) and DME (5 mL), and then the reaction was degassed by argon for 10 min. To the reaction mixture was added [1,1′-bis(diphenylphosphino)ferrocene]dichloropalladium(II) dichloromethane complex (43.6 mg, 0.053 mmol, 0.1 eq). After the solution was stirred at 90° C. for 16 h, the mixture was treated with water and extracted with ethyl acetate three times. The combined organic layers were dried over Na₂SO₄₍ₛ₎, filtered, and concentrated in vacuo to give a residue, which was purified by silica gel column chromatography (n-hexane:EtOAc=3:1 to 1:1), followed by slurry (with methanol) to give P119 (130 mg, 55% yield) as a white solid. ¹H NMR (400 MHz, CDCl₃), δ: 8.33 (dd, J=1.8, 0.4 Hz, 1H), 7.53 (d, J=8.8, 0.4 Hz, 1H), 7.41 (dd, J=8.8, 1.8 Hz, 1H), 7.28 (s, 1H), 5.71 (dd, J=10.0, 2.8 Hz, 1H), 4.12-4.05 (m, 1H), 3.94-3.87 (m, 7H), 3.81-3.73 (m, 1H), 3.58-3.52 (m, 4H), 2.67-2.58 (m, 1H), 2.56 (s, 3H), 2.23-2.13 (m, 1H), 2.11-2.03 (m, 1H), 1.85-1.74 (m, 2H), 1.71-1.64 (m, 1H).

### Preparation 120. 2-Methyl-6-(5-(methylsulfonyl)-1-(tetrahydro-2H-pyran-2-yl)-1H-indazol-3-yl)-4-morpholinopyridazin-3(2H)-one (P120)

To a solution of P119 (80.0 mg, 0.181 mmol, 1.0 eq) in MeOH (2 mL) was added sodium tungstate dihydrate (3.0 mg, 0.009 mmol, 0.05 eq) and 30% hydrogen peroxide aqueous solution (1.0 mL) at 0° C. The reaction mixture was stirred at rt for 4 h. The resulting precipitate was collected through filtration and dried under air to give P120 (81 mg, 94% yield) as a white solid. ¹H NMR (400 MHz, CDCl₃), δ: 9.06 (s, 1H), 7.96 (d, J=8.8 Hz, 1H), 7.77 (d, J=8.8 Hz, 1H), 7.25 (s, 1H), 5.79 (dd, J=9.6, 2.4 Hz, 1H), 4.13-4.05 (m, 1H), 3.97-3.87 (m, 7H), 3.84-3.75 (m, 1H), 3.62-3.53 (m, 4H), 3.11 (s, 3H), 2.68-2.54 (m, 1H), 2.25-2.08 (m, 2H), 1.86-1.68 (m, 3H).

### Synthesis of 2-methyl-6-(5-(methylsulfonyl)-1-(tetrahydro-2H-pyran-2-yl)-1H-indazol-3-yl)-4-morpholinopyridazin-3(2H)-one (P125)

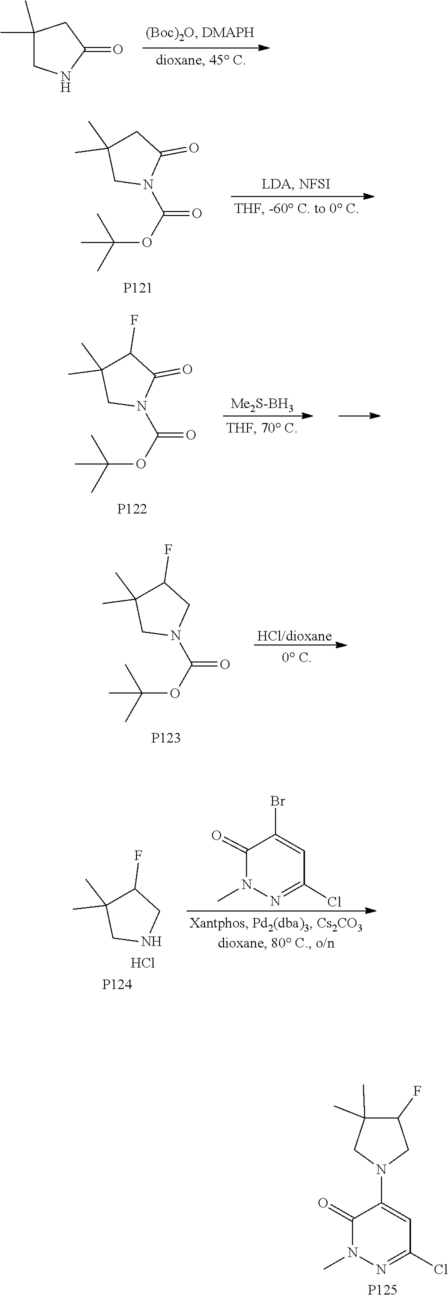

### Preparation 121. tert-Butyl 4,4-dimethyl-2-oxopyrrolidine-1-carboxylate (P121)

To a solution of 4,4-dimethylpyrrolidin-2-one (3.7 g, 32.7 mmol) in dioxane (89 mL) was added di-tert-butyl dicarbonate (9.0 mL, 39 mmol) and 4-dimethylaminopyridine (4.80 g, 39.2 mmol) at rt. After stirred at 45° C. for 2 h, the reaction mixture was cooled to rt, diluted with water (100 mL) and extracted with ethyl acetate (100 mL) for three times. The combined organic layers were washed with 3 M HCl_{(aq)} (100 mL), dried over Na₂SO₄₍ₛ₎, filtered, and concentrated to afford the desired product (7.17 g, crude) which was used in the next step without further purification. ¹H NMR (400 MHz, CDCl₃), δ: 3.48-3.40 (m, 2H), 2.36-2.27 (m, 2H), 1.56-1.46 (m, 9H), 1.20-1.09 (m, 6H).

### Preparation 122. tert-Butyl 3-fluoro-4,4-dimethyl-2-oxopyrrolidine-1-carboxylate (P122)

To a solution of tert-butyl 4,4-dimethyl-2-oxopyrrolidine-1-carboxylate (P121) (8.17 g, 38.3 mmol) in THF (204 mL) was added 2 M lithium diisopropylamide in tetrahydrofuran (25 mL, 50 mmol) at −60° C. After the mixture was stirred for 1 h at −60° C., N-fluorobenzenesulfonimide (15.7 g, 49.8 mmol) was added dropwise and the reaction mixture was stirred at 0° C. for another 2 h. The reaction mixture was quenched with sat. NH₄Cl_{(aq)} (30 mL). The mixture was extracted with ethyl acetate, dried over Na₂SO₄₍ₛ₎, filtered, and concentrated. The residue was purified by silica gel column chromatography (petroleum ether:ethyl acetate=10:0 to 0:10) to afford the desired product (7.67 g, 90% purity, 87% yield). ¹H NMR (400 MHz, CDCl₃), δ: 4.64 (d, J=51.6 Hz, 1H), 3.55-3.53 (m, 1H), 3.30 (d, J=10.8 Hz, 1H), 1.52 (s, 9H), 1.24 (s, 3H), 1.11 (d, J=2.4 Hz, 3H).

### Preparation 123. tert-Butyl 4-fluoro-3,3-dimethylpyrrolidine-1-carboxylate (P123)

To a solution of tert-butyl 3-fluoro-4,4-dimethyl-2-oxopyrrolidine-1-carboxylate P122 (5.89 mg, 25.5 mmol) in THF (65 mL) was added dimethyl sulfide borane (26 mL, 51 mmol, 2 M in THF) at rt. After the solution was stirred at 70° C. for 16 h, the reaction mixture was cooled to rt, quenched with methanol (100 mL) and concentrated in vacuo. The residue was diluted with water (50 mL) and extracted with ethyl acetate (80 mL) twice. The combined organic layers were dried over Na₂SO₄₍ₛ₎, filtered, and concentrated in vacuo. The crude product was purified by silica gel column chromatography (petroleum ether:ethyl acetate=10:0 to 10:1) to afford the desired product (2.03 g, 23% yield) as colorless liquid. ¹H NMR (400 MHz, CDCl₃), δ: 4.82-4.40 (m, 1H), 3.81-2.79 (m, 5H), 1.70-1.37 (m, 9H), 1.23-0.97 (m, 6H).

### Preparation 124. 4-Fluoro-3,3-dimethylpyrrolidine hydrochloride (P124)

To a solution of tert-butyl 4-fluoro-3,3-dimethylpyrrolidine-1-carboxylate (P123, 500 mg, 2.30 mmol) in dioxane (4 mL) was added 4M hydrogen chloride (2 mL) at 0° C. After stirred at 0° C. for 2 h, the reaction mixture was concentrated to dryness to afford the desired product (350 mg, 70% purity, 77% yield) as colorless oil. LCMS (ESI): R_{T}=0.217 min, mass calc. for C₆H₁₃ClFN 153.1, m/z found 118.0 [M+H-HCl]⁺.

### Preparation 125. 6-Chloro-4-(4-fluoro-3,3-dimethylpyrrolidin-1-yl)-2-methylpyridazin-3(2H)-one (P125)

To a solution of 4-fluoro-3,3-dimethylpyrrolidine hydrochloride (P124, 350 mg, 2.28 mmol) in anh. 1,4-dioxane (6 mL) was added 4-bromo-6-chloro-2-methylpyridazin-3(2H)-one (610 mg, 2.73 mmol), cesium carbonate (1.5 g, 4.56 mmol), and 9,9-dimethyl-4,5-bis(diphenylphosphino)xanthene (130 mg, 0.23 mmol). The mixture was degassed with nitrogen for 15 min. The mixture was added tris(dibenzylideneacetone)dipalladium (100 mg, 0.12 mmol) and degassed with nitrogen. After stirred at 80° C. for 16 h, the reaction mixture was filtered through a pad of celite. The filtrate was treated with water (15 mL) and extracted with ethyl acetate (15 mL) for three times. The combined organic layers were dried over Na₂SO₄₍ₛ₎, filtered, and concentrated. The residue was purified by silica gel chromatography (petroleum ether:ethyl acetate=4:1) to give the desired product (260 mg, 88% purity, 44% yield) as yellow solids. LCMS (ESI): R_{T}=1.470 min, mass calc. for C₁₁H₁₅C₁FN₃O 259.1, m/z found 260.1 [M+H]⁺.

### Synthesis of 2-methyl-6-(5-(methylsulfonyl)-1-(tetrahydro-2H-pyran-2-yl)-1H-indazol-3-yl)-4-morpholinopyridazin-3(2H)-one (P127)

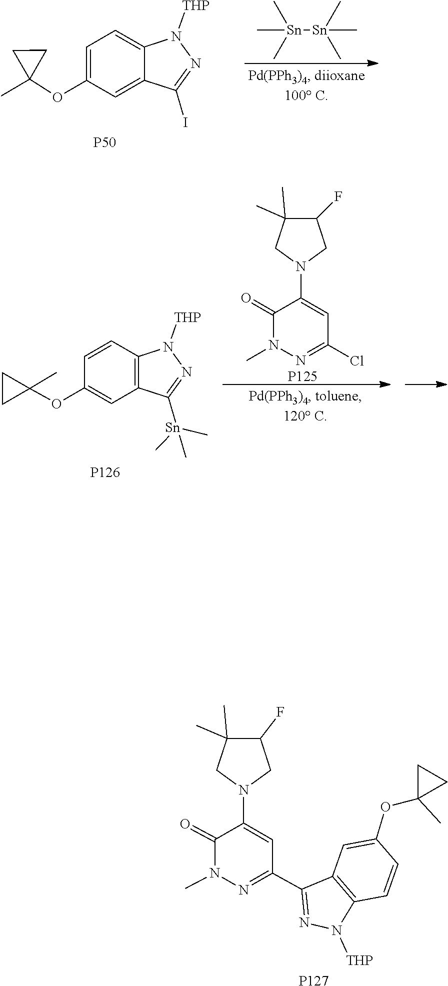

### Preparation 126. 5-(1-Methylcyclopropoxy)-1-(tetrahydro-2H-pyran-2-yl)-3-(trimethylstannyl)-1H-indazole (P126)

To a solution of 3-iodo-5-(1-methylcyclopropoxy)-1-(tetrahydro-2H-pyran-2-yl)-1H-indazole (P50, 2.0 g, 5.02 mmol) in dioxane (20 mL) was added 1,1,1,2,2,2-hexamethyldistannane (8.23 g, 25.11 mmol) and tetrakis(triphenylphosphine)palladium (580 mg, 0.50 mmol) under nitrogen atmosphere. After stirred at 100° C. for 16 h, the reaction mixture was diluted with water (20 mL) and extracted with ethyl acetate (20 mL) for three times. The combined organic layers were dried over Na₂SO₄₍ₛ₎, filtered, and concentrated to leave a residue which was purified by silica gel column chromatography (petroleum ether:ethyl acetate=10:1) to afford the desired product P126 (1.4 g, 90% purity, 60% yield) as yellow oil. LCMS (ESI): R_{T}=2.018 min, mass calc. for C₁₉H₁₈N₂O₂Sn 434.1, m/z found 435.1 [M+H]⁺.

### Preparation 127. 4-(4-Fluoro-3,3-dimethylpyrrolidin-1-yl)-2-methyl-6-(5-(1-methylcyclopropoxy)-1-(tetrahydro-2H-pyran-2-yl)-1H-indazol-3-yl)pyridazin-3(2H)-one (P127)

To a solution of 5-(1-methylcyclopropoxy)-1-(tetrahydro-2H-pyran-2-yl)-3-(trimethylstannyl)-1H-indazole (P126, 100 mg, 0.23 mmol) and 6-chloro-4-(4-fluoro-3,3-dimethylpyrrolidin-1-yl)-2-methylpyridazin-3(2H)-one (P125, 72 mg, 0.27 mmol) in toluene (3 mL) was added tetrakis(triphenylphosphine)palladium (26 mg, 0.026 mmol) at rt under nitrogen atmosphere. After stirred at 120° C. for 16 h, the reaction mixture was cooled to rt, diluted with water (10 mL) and extracted with ethyl acetate (20 mL) for three times. The combined organic layers were dried over Na₂SO₄₍ₛ₎, filtered, and concentrated to leave a residue which was purified by silica gel column chromatography (petroleum ether:ethyl acetate=10:1) to afford the desired product (75 mg, 59%) as a yellow oil. ¹H NMR (400 MHz, DMSO-d₆), δ: 8.02 (d, J=2.8 Hz, 1H), 7.70 (d, J=12.4 Hz, 1H), 7.12 (dd, J=12.4, 2.8 Hz, 1H), 6.72 (s, 1H), 5.89-5.86 (m, 1H), 4.85 (dd, J=71.2, 4.0 Hz, 1H), 4.34-4.16 (m, 1H), 4.08-3.94 (m, 2H), 3.82-3.69 (m, 5H), 3.47-3.44 (m, 1H), 2.46-2.29 (m, 1H), 2.10-2.00 (m, 2H), 1.81-1.77 (m, 1H), 1.66-1.62 (m, 2H), 1.58 (s, 3H), 1.20 (s, 3H), 1.05 (s, 3H), 1.01-0.97 (m, 2H), 0.84-0.81 (in, 2H).

In the Table 3 presented certain examples of key intermediates.

TABLE 3
Certain examples of key intermediates.

| # | Structure |
|---|---|
| P7 | 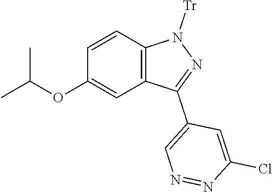 |
| P8 | 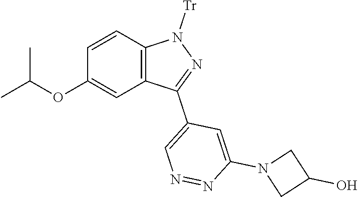 |
| P9 | 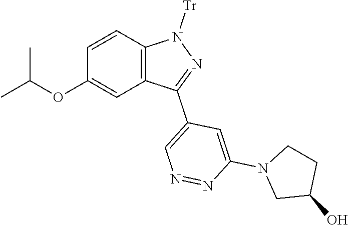 |
| P10 | 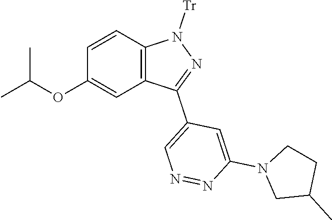 |
| P11 | 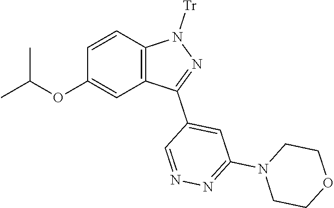 |
| P12 | 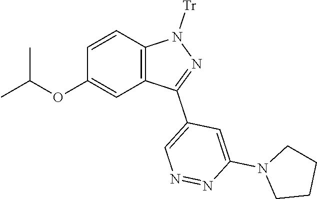 |
| P17 | 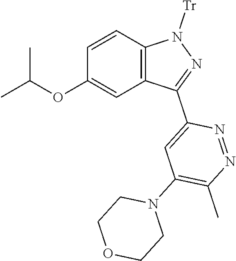 |
| P24 | 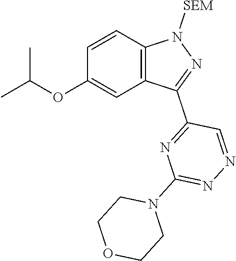 [SMILES: CC(C)Oc1ccc2c(c1)c(-c1cnnc(N3CCOCC3)n1)nn2COCC[Si](C)(C)C] |
| P29 | 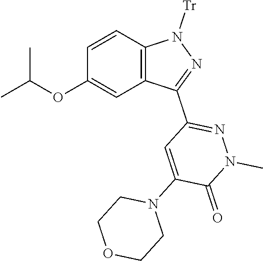 |
| P33 | 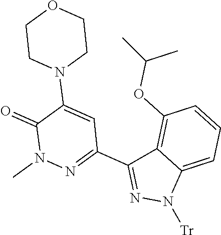 |
| P35 | 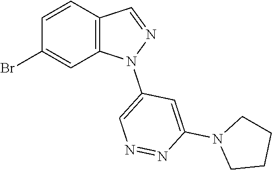 [SMILES: Brc1ccc2cnn(-c3cnnc(N4CCCC4)c3)c2c1] |
| P36 | 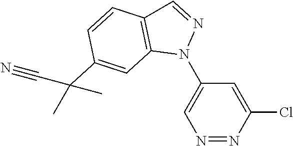 [SMILES: CC(C)(C#N)c1ccc2cnn(-c3cnnc(Cl)c3)c2c1] |
| P37 | 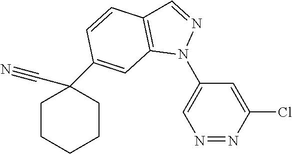 [SMILES: N#CC1(c2ccc3cnn(-c4cnnc(Cl)c4)c3c2)CCCCC1] |
| P38 | 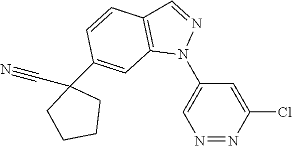 [SMILES: N#CC1(c2ccc3cnn(-c4cnnc(Cl)c4)c3c2)CCCC1] |
| P42 | 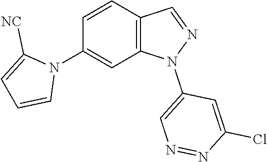 [SMILES: N#Cc1cccn1-c1ccc2cnn(-c3cnnc(Cl)c3)c2c1] |
| P43 | 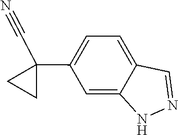 [SMILES: N#CC1(c2ccc3cn[nH]c3c2)CC1] |
| P28 | 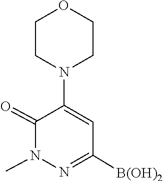 [SMILES: Cn1nc(B(O)O)cc(N2CCOCC2)c1=O] |
| P44 | 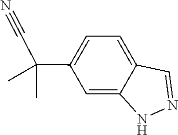 |

### Examples of the Final Compound

In the Table 4 presented certain non-limiting examples of the compound of Formula (I).

TABLE 4
Selected examples of the compound of Formula (I)

| | | | [MH]⁺ | [MH]⁺ |
|---|---|---|---|---|
| # | Structure | IUPAC Name | Calc. | Found |
| 1 | 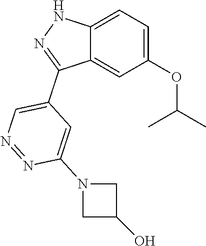 [SMILES: CC(C)Oc1ccc2[nH]nc(-c3cnnc(N4CC(O)C4)c3)c2c1] | 1-[5-(5-isopropoxy-1H- indazol-3-yl)pyridazin-3- yl]azetidin-3-ol | 326.162 | 326.3 |
| 2 | 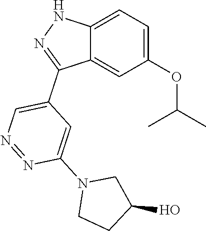 [SMILES: CC(C)Oc1ccc2[nH]nc(-c3cnnc(N4CC[C@H](O)C4)c3)c2c1] | (R)-1-(5-(5-isopropoxy-1H- indazol-3-yl)pyridazin-3- yl)pyrrolidin-3-ol | 340.177 | 340.3 |
| 3 | 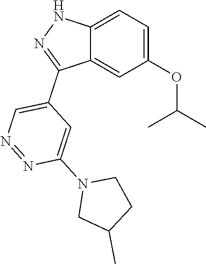 [SMILES: CC1CCN(c2cc(-c3n[nH]c4ccc(OC(C)C)cc34)cnn2)C1] | 5-isopropoxy-3-[6-(3- methylpyrrolidin-1- yl)pyridazin-4-yl]-1H-indazole | 338.198 | 338.3 |
| 4 | 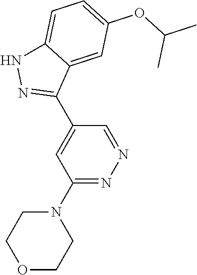 [SMILES: CC(C)Oc1ccc2[nH]nc(-c3cnnc(N4CCOCC4)c3)c2c1] | 4-[5-(5-isopropoxy-1H- indazol-3-yl)pyridazin-3- yl]morpholine | 342.184 | 342.2 |
| 5 | 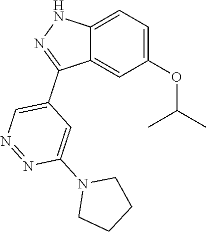 [SMILES: CC(C)Oc1ccc2[nH]nc(-c3cnnc(N4CCCC4)c3)c2c1] | 5-isopropoxy-3-(6-pyrrolidin- 1-ylpyridazin-4-yl)-1H- indazole | 324.182 | 324.2 |
| 6 | 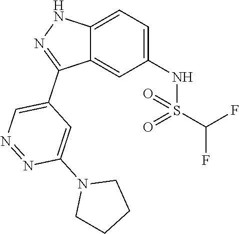 [SMILES: O=S(=O)(Nc1ccc2[nH]nc(-c3cnnc(N4CCCC4)c3)c2c1)C(F)F] | 1,1-difluoro-N-[3-(6- pyrrolidin-1-ylpyridazin-4-yl)- 1H-indazol-5- yl]methanesulfonamide | 395.11 | 395.2 |
| 7 | 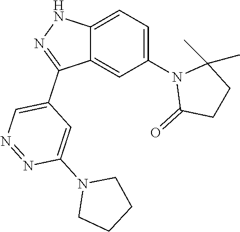 [SMILES: CC1(C)CCC(=O)N1c1ccc2[nH]nc(-c3cnnc(N4CCCC4)c3)c2c1] | 5,5-dimethyl-1-[3-(6- pyrrolidin-1-ylpyridazin-4-yl)- 1H-indazol-5-yl]pyrrolidin-2- one | 377.209 | 377.3 |
| 8 | 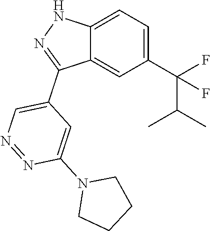 [SMILES: CC(C)C(F)(F)c1ccc2[nH]nc(-c3cnnc(N4CCCC4)c3)c2c1] | 5-(1,1-difluoro-2-methyl- propyl)-3-(6-pyrrolidin-1- ylpyridazin-4-yl)-1H-indazole | 358.184 | 358.3 |
| 9 | 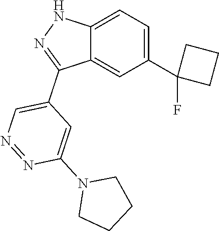 [SMILES: FC1(c2ccc3[nH]nc(-c4cnnc(N5CCCC5)c4)c3c2)CCC1] | 5-(1-fluorocyclobutyl)-3-(6- pyrrolidin-1-ylpyridazin-4-yl)- 1H-indazole | 338.178 | 338.3 |
| 10 | 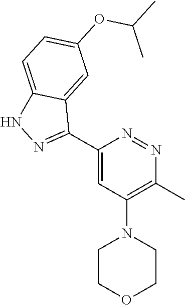 [SMILES: Cc1nnc(-c2n[nH]c3ccc(OC(C)C)cc23)cc1N1CCOCC1] | 4-[6-(5-isopropoxy-1H- indazol-3-yl)-3-methyl- pyridazin-4-yl]morpholine | 354.193 | 354.2 |
| 11 | 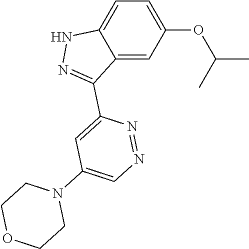 [SMILES: CC(C)Oc1ccc2[nH]nc(-c3cc(N4CCOCC4)cnn3)c2c1] | 4-[6-(5-isopropoxy-1H- indazol-3-yl)pyridazin-4- yl]morpholine | 340.177 | 340.2 |
| 12 | 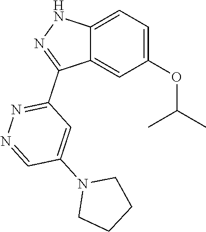 [SMILES: CC(C)Oc1ccc2[nH]nc(-c3cc(N4CCCC4)cnn3)c2c1] | 5-isopropoxy-3-(5-pyrrolidin- 1-ylpyridazin-3-yl)-1H- indazole | 324.182 | 324.3 |
| 13 | 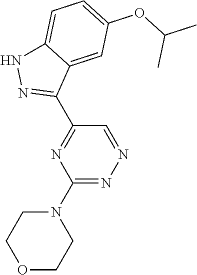 [SMILES: CC(C)Oc1ccc2[nH]nc(-c3cnnc(N4CCOCC4)n3)c2c1] | 4-[5-(5-isopropoxy-1H- indazol-3-yl)-1,2,4-triazin-3- yl]morpholine | 341.173 | 341.2 |
| 14 | 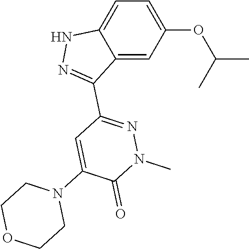 [SMILES: CC(C)Oc1ccc2[nH]nc(-c3cc(N4CCOCC4)c(=O)n(C)n3)c2c1] | 6-(5-isopropoxy-1H-indazol-3- yl)-2-methyl-4-morpholino- pyridazin-3-one | 370.188 | 370.2 |
| 15 | 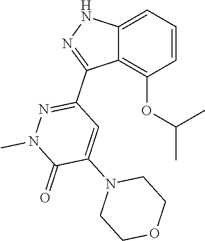 [SMILES: CC(C)Oc1cccc2[nH]nc(-c3cc(N4CCOCC4)c(=O)n(C)n3)c12] | 6-(4-isopropoxy-1H-indazol-3- yl)-2-methyl-4-morpholino- pyridazin-3-one | 370.188 | 370.5 |
| 16 | 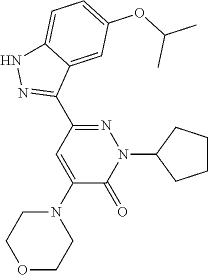 [SMILES: CC(C)Oc1ccc2[nH]nc(-c3cc(N4CCOCC4)c(=O)n(C4CCCC4)n3)c2c1] | 2-cyclopentyl-6-(5- isopropoxy-1H-indazol-3-yl)- 4-morpholino-pyridazin-3-one | 424.235 | 424.6 |
| 17 | 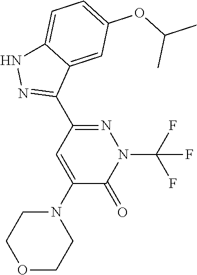 [SMILES: CC(C)Oc1ccc2[nH]nc(-c3cc(N4CCOCC4)c(=O)n(C(F)(F)F)n3)c2c1] | 6-(5-isopropoxy-1H-indazol-3- yl)-4-morpholino-2- (trifluoromethyl)pyridazin-3- one | 424.16 | 424 |
| 18 | 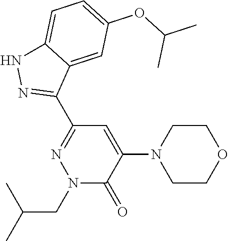 [SMILES: CC(C)Cn1nc(-c2n[nH]c3ccc(OC(C)C)cc23)cc(N2CCOCC2)c1=O] | 2-isobutyl-6-(5-isopropoxy- 1H-indazol-3-yl)-4- morpholino-pyridazin-3-one | 412.235 | 412.3 |
| 19 | 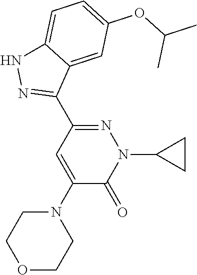 [SMILES: CC(C)Oc1ccc2[nH]nc(-c3cc(N4CCOCC4)c(=O)n(C4CC4)n3)c2c1] | 2-cyclopropyl-6-(5- isopropoxy-1H-indazol-3-yl)- 4-morpholino-pyridazin-3-one | 396.204 | 396.2 |
| 20 | 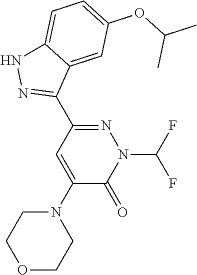 [SMILES: CC(C)Oc1ccc2[nH]nc(-c3cc(N4CCOCC4)c(=O)n(C(F)F)n3)c2c1] | 2-(difluoromethyl)-6-(5- isopropoxy-1H-indazol-3-yl)- 4-morpholino-pyridazin-3-one | 406.169 | 406.2 |
| 21 | 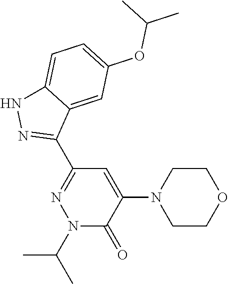 [SMILES: CC(C)Oc1ccc2[nH]nc(-c3cc(N4CCOCC4)c(=O)n(C(C)C)n3)c2c1] | 6-(5-isopropoxy-1H-indazol-3- yl)-2-isopropyl-4-morpholino- pyridazin-3-one | 398.219 | 398.3 |
| 22 | 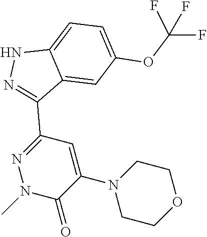 [SMILES: Cn1nc(-c2n[nH]c3ccc(OC(F)(F)F)cc23)cc(N2CCOCC2)c1=O] | 2-methyl-4-morpholino-6-[5- (trifluoromethoxy)-1H- indazol-3-yl]pyridazin-3-one | 396.128 | 396.3 |
| 23 | 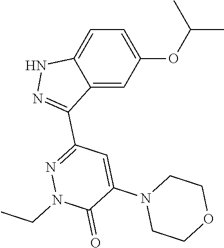 [SMILES: CCn1nc(-c2n[nH]c3ccc(OC(C)C)cc23)cc(N2CCOCC2)c1=O] | 2-ethyl-6-(5-isopropoxy-1H- indazol-3-yl)-4-morpholino- pyridazin-3-one | 384.204 | 384.3 |
| 24 | 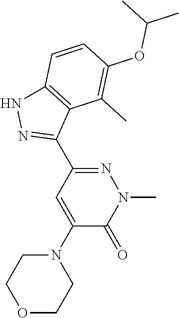 [SMILES: Cc1c(OC(C)C)ccc2[nH]nc(-c3cc(N4CCOCC4)c(=O)n(C)n3)c12] | 6-(5-isopropoxy-4-methyl-1H- indazol-3-yl)-2-methyl-4- morpholino-pyridazin-3-one | 384.204 | 384.5 |
| 25 | 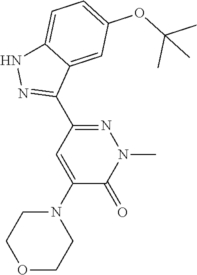 [SMILES: Cn1nc(-c2n[nH]c3ccc(OC(C)(C)C)cc23)cc(N2CCOCC2)c1=O] | 6-(5-tert-butoxy-1H-indazol-3- yl)-2-methyl-4-morpholino- pyridazin-3-one | 384.204 | 384.2 |
| 26 | 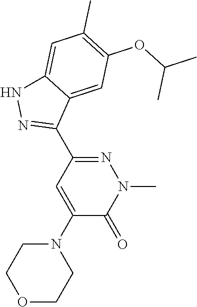 [SMILES: Cc1cc2[nH]nc(-c3cc(N4CCOCC4)c(=O)n(C)n3)c2cc1OC(C)C] | 6-(5-isopropoxy-6-methyl-1H- indazol-3-yl)-2-methyl-4- morpholino-pyridazin-3-one | 384.204 | 384.3 |
| 27 | 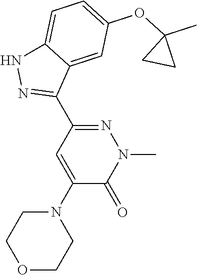 [SMILES: Cn1nc(-c2n[nH]c3ccc(OC4(C)CC4)cc23)cc(N2CCOCC2)c1=O] | 2-methyl-6-[5-(1- methylcyclopropoxy)-1H- indazol-3-yl]-4-morpholino- pyridazin-3-one | 382.188 | 382.5 |
| 28 | 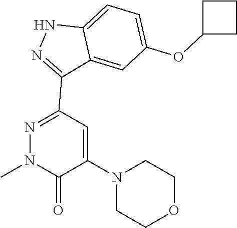 [SMILES: Cn1nc(-c2n[nH]c3ccc(OC4CCC4)cc23)cc(N2CCOCC2)c1=O] | 6-[5-(cyclobutoxy)-1H- indazol-3-yl]-2-methyl-4- morpholino-pyridazin-3-one | 382.188 | 382.4 |
| 29 | 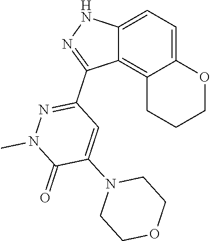 [SMILES: Cn1nc(-c2n[nH]c3ccc4c(c23)CCCO4)cc(N2CCOCC2)c1=O] | 2-methyl-4-morpholino-6- (3,7,8,9-tetrahydropyrano[3,2- e]indazol-1-yl)pyridazin-3-one | 368.172 | 368.3 |
| 30 | 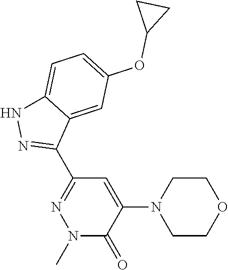 [SMILES: Cn1nc(-c2n[nH]c3ccc(OC4CC4)cc23)cc(N2CCOCC2)c1=O] | 6-[5-(cyclopropoxy)-1H- indazol-3-yl]-2-methyl-4- morpholino-pyridazin-3-one | 368.172 | 368.3 |
| 31 | 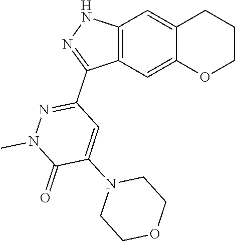 [SMILES: Cn1nc(-c2n[nH]c3cc4c(cc23)OCCC4)cc(N2CCOCC2)c1=O] | 2-methyl-4-morpholino-6- (1,6,7,8-tetrahydropyrano[2,3- flindazol-3-yl)pyridazin-3-one | 368.172 | 368.2 |
| 32 | 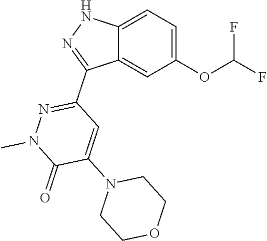 [SMILES: Cn1nc(-c2n[nH]c3ccc(OC(F)F)cc23)cc(N2CCOCC2)c1=O] | 6-[5-(difluoromethoxy)-1H- indazol-3-yl]-2-methyl-4- morpholino-pyridazin-3-one | 378.138 | 378.0 |
| 33 | 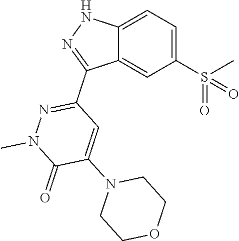 [SMILES: Cn1nc(-c2n[nH]c3ccc(S(C)(=O)=O)cc23)cc(N2CCOCC2)c1=O] | 2-methyl-6-(5-methylsulfonyl- 1H-indazol-3-yl)-4- morpholino-pyridazin-3-one | 390.124 | 390.1 |
| 34 | 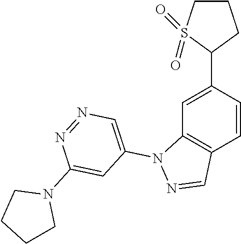 [SMILES: O=S1(=O)CCCC1c1ccc2cnn(-c3cnnc(N4CCCC4)c3)c2c1] | 2-[1-(6-pyrrolidin-1- ylpyridazin-4-yl)indazol-6- yl]thiolane 1,1-dioxide | 384.149 | 384.2 |
| 35 | 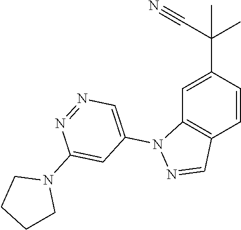 [SMILES: CC(C)(C#N)c1ccc2cnn(-c3cnnc(N4CCCC4)c3)c2c1] | 2-methyl-2-[1-(6-pyrrolidin-1- ylpyridazin-4-yl)indazol-6- yl]propanenitrile | 333.183 | 333.7 |
| 36 | 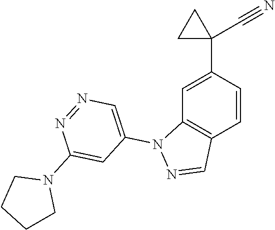 [SMILES: N#CC1(c2ccc3cnn(-c4cnnc(N5CCCC5)c4)c3c2)CC1] | 1-[1-(6-pyrrolidin-1- ylpyridazin-4-yl)indazol-6- yl]cyclopropanecarbonitrile | 331.167 | 331.3 |
| 37 | 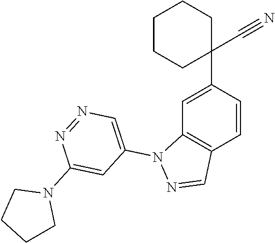 [SMILES: N#CC1(c2ccc3cnn(-c4cnnc(N5CCCC5)c4)c3c2)CCCCC1] | 1-[1-(6-pyrrolidin-1- ylpyridazin-4-yl)indazol-6- yl]cyclohexanecarbonitrile | 373.214 | 373.6 |
| 38 | 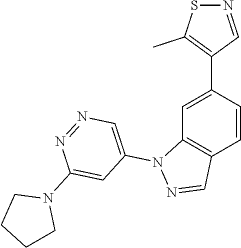 [SMILES: Cc1sncc1-c1ccc2cnn(-c3cnnc(N4CCCC4)c3)c2c1] | 5-methyl-4-[1-(6-pyrrolidin-1- ylpyridazin-4-yl)indazol-6- yl]isothiazole | 363.139 | 363.25 |
| 39 | 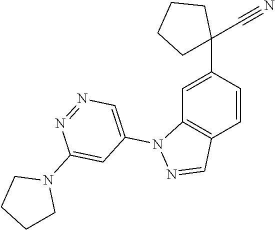 [SMILES: N#CC1(c2ccc3cnn(-c4cnnc(N5CCCC5)c4)c3c2)CCCC1] | 1-[1-(6-pyrrolidin-1- ylpyridazin-4-yl)indazol-6- yl]cyclopentanecarbonitrile | 359.198 | 359.7 |
| 40 | 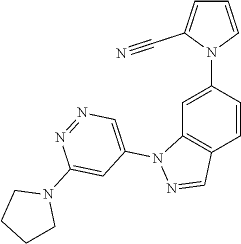 [SMILES: N#Cc1cccn1-c1ccc2cnn(-c3cnnc(N4CCCC4)c3)c2c1] | 1-[1-(6-pyrrolidin-1- ylpyridazin-4-yl)indazol-6- yl]pyrrole-2-carbonitrile | 356.162 | 356.2 |
| 41 | 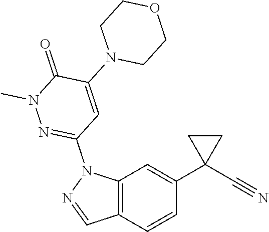 [SMILES: Cn1nc(-n2ncc3ccc(C4(C#N)CC4)cc32)cc(N2CCOCC2)c1=O] | 1-[1-(1-methyl-5-morpholino- 6-oxo-pyridazin-3-yl)indazol- 6-yl]cyclopropanecarbonitrile | 377.173 | 377.0 |
| 42 | 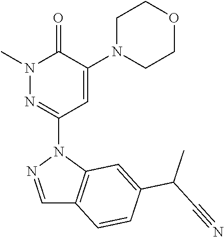 [SMILES: CC(C#N)c1ccc2cnn(-c3cc(N4CCOCC4)c(=O)n(C)n3)c2c1] | 2-[1-(1-methyl-5-morpholino- 6-oxo-pyridazin-3-yl)indazol- 6-yl]propanenitrile | 365.173 | 365.3 |
| 43 | 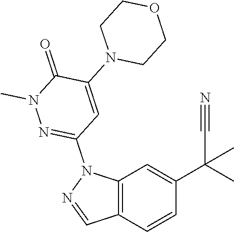 [SMILES: Cn1nc(-n2ncc3ccc(C(C)(C)C#N)cc32)cc(N2CCOCC2)c1=O] | 2-methyl-2-[1-(1-methyl-5- morpholino-6-oxo-pyridazin- 3-yl)indazol-6- yl]propanenitrile | 379.188 | 379.0 |
| 44 | 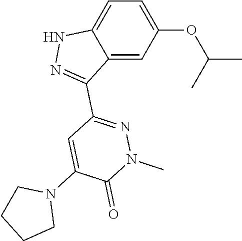 [SMILES: CC(C)Oc1ccc2[nH]nc(-c3cc(N4CCCC4)c(=O)n(C)n3)c2c1] | 6-(5-isopropoxy-1H-indazol-3- yl)-2-methyl-4-pyrrolidin-1-yl- pyridazin-3-one | 354.193 | 354.3 |
| 45 | 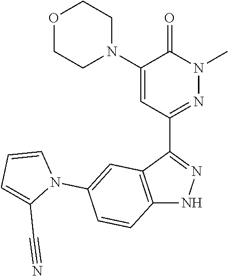 [SMILES: Cn1nc(-c2n[nH]c3ccc(-n4cccc4C#N)cc23)cc(N2CCOCC2)c1=O] | 1-[3-(1-methyl-5-morpholino- 6-oxo-pyridazin-3-yl)-1H- indazol-5-yl]pyrrole-2- carbonitrile | 402.168 | 402.2 |
| 46 | 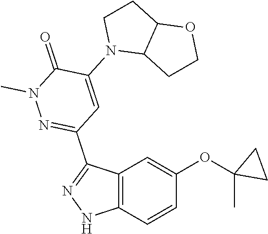 [SMILES: Cn1nc(-c2n[nH]c3ccc(OC4(C)CC4)cc23)cc(N2CCC3OCCC32)c1=O] | 4-(2,3,3a,5,6,6a- hexahydrofuro[3,2-b]pyrrol-4- yl)-2-methyl-6-[5-(1- methylcyclopropoxy)-1H- indazol-3-yl]pyridazin-3-one | 408.204 | 408.3 |
| 47 | 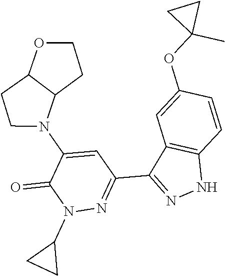 [SMILES: CC1(Oc2ccc3[nH]nc(-c4cc(N5CCC6OCCC65)c(=O)n(C5CC5)n4)c3c2)CC1] | 4-(2,3,3a,5,6,6a- hexahydrofuro[3,2-b]pyrrol-4- yl)-2-cyclopropyl-6-[5-(1- methylcyclopropoxy)-1H- indazol-3-yl]pyridazin-3-one | 434.219 | 434.3 |
| 48 | 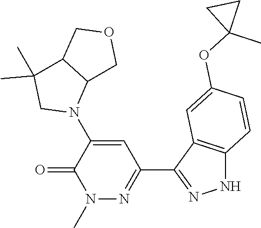 | 4-(3,3-dimethyl-3a,4,6,6a- tetrahydro-2H-furo[3,4- b]pyrrol-1-yl)-2-methyl-6-[5- (1-methylcyclopropoxy)-1H- indazol-3-yl]pyridazin-3-one | 436.235 | 436.3 |
| 49 | 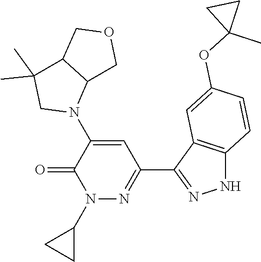 | 2-cyclopropyl-4-(3,3-dimethyl- 3a,4,6,6a-tetrahydro-2H- furo[3,4-b]pyrrol-1-yl)-6-[5- (1-methylcyclopropoxy)-1H- indazol-3-yl]pyridazin-3-one | 462.251 | 462.3 |
| 50 | 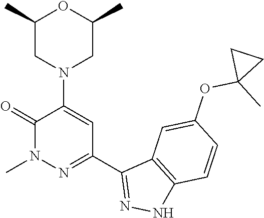 [SMILES: C[C@@H]1CN(c2cc(-c3n[nH]c4ccc(OC5(C)CC5)cc34)nn(C)c2=O)C[C@H](C)O1] | 4-[(2S,6R)-2,6- dimethylmorpholin-4-yl]-2- methyl-6-[5-(1- methylcyclopropoxy)-1H- indazol-3-yl]pyridazin-3-one | 410.219 | 410.3 |
| 51 | 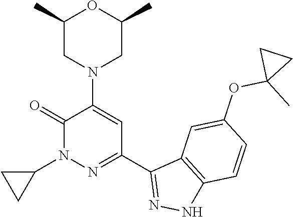 [SMILES: C[C@@H]1CN(c2cc(-c3n[nH]c4ccc(OC5(C)CC5)cc34)nn(C3CC3)c2=O)C[C@H](C)O1] | 2-cyclopropyl-4-[(2S,6R)-2,6- dimethylmorpholin-4-yl]-6-[5- (1-methylcyclopropoxy)-1H- indazol-3-yl]pyridazin-3-one | 436.235 | 436.3 |
| 52 | 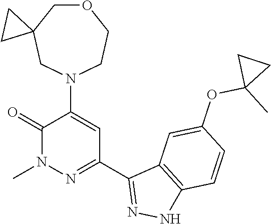 [SMILES: Cn1nc(-c2n[nH]c3ccc(OC4(C)CC4)cc23)cc(N2CCOCC3(CC3)C2)c1=O] | 2-methyl-6-[5-(1- methylcyclopropoxy)-1H- indazol-3-yl]-4-(5-oxa-8- azaspiro[2.6]nonan-8- yl)pyridazin-3-one | 422.219 | 422.3 |
| 53 | 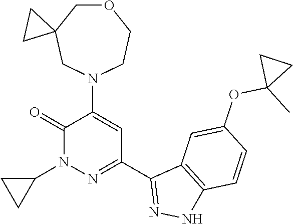 [SMILES: CC1(Oc2ccc3[nH]nc(-c4cc(N5CCOCC6(CC6)C5)c(=O)n(C5CC5)n4)c3c2)CC1] | 2-cyclopropyl-6-[5-(1- methylcyclopropoxy)-1H- indazol-3-yl]-4-(5-oxa-8- azaspiro[2.6]nonan-8- yl)pyridazin-3-one | 448.235 | 448.3 |
| 54 | 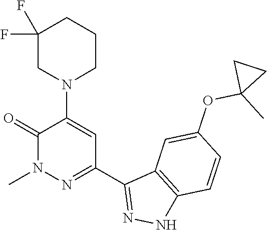 [SMILES: Cn1nc(-c2n[nH]c3ccc(OC4(C)CC4)cc23)cc(N2CCCC(F)(F)C2)c1=O] | 4-(3,3-difluoro-1-piperidyl)-2- methyl-6-[5-(1- methylcyclopropoxy)-1H- indazol-3-yl]pyridazin-3-one | 416.190 | 416.3 |
| 55 | 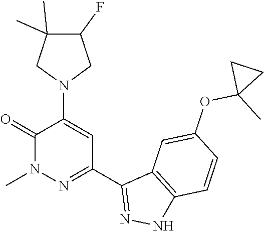 [SMILES: Cn1nc(-c2n[nH]c3ccc(OC4(C)CC4)cc23)cc(N2CC(F)C(C)(C)C2)c1=O] | 4-(4-fluoro-3,3-dimethyl- pyrrolidin-1-yl)-2-methyl-6-[5- (1-methylcyclopropoxy)-1H- indazol-3-yl]pyridazin-3-one | 412.215 | 412.1 |
| 56 | 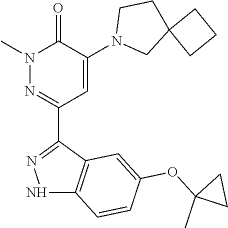 [SMILES: Cn1nc(-c2n[nH]c3ccc(OC4(C)CC4)cc23)cc(N2CCC3(CCC3)C2)c1=O] | 4-(6-azaspiro[3.4]octan-6-yl)- 2-methyl-6-[5-(1- methylcyclopropoxy)-1H- indazol-3-yl]pyridazin-3-one | 406.224 | 406.3 |
| 57 | 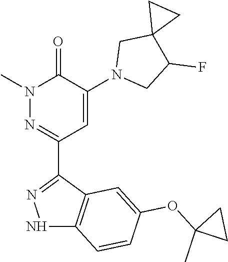 [SMILES: Cn1nc(-c2n[nH]c3ccc(OC4(C)CC4)cc23)cc(N2CC(F)C3(CC3)C2)c1=O] | 4-(7-fluoro-5- azaspiro[2.4]heptan-5-yl)-2- methyl-6-[5-(1- methylcyclopropoxy)-1H- indazol-3-yl]pyridazin-3-one | 410.199 | 410.2 |
| 58 | 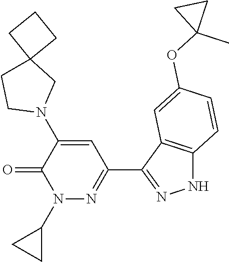 [SMILES: CC1(Oc2ccc3[nH]nc(-c4cc(N5CCC6(CCC6)C5)c(=O)n(C5CC5)n4)c3c2)CC1] | 4-(6-azaspiro[3.4]octan-6-yl)- 2-cyclopropyl-6-[5-(1- methylcyclopropoxy)-1H- indazol-3-yl]pyridazin-3-one | 432.240 | 432.4 |
| 59 | 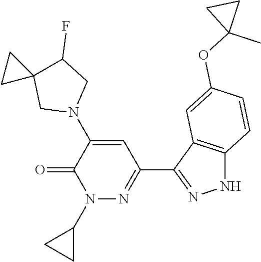 [SMILES: CC1(Oc2ccc3[nH]nc(-c4cc(N5CC(F)C6(CC6)C5)c(=O)n(C5CC5)n4)c3c2)CC1] | 2-cyclopropyl-4-(7-fluoro-5- azaspiro[2.4]heptan-5-yl)-6-[5- (1-methylcyclopropoxy)-1H- indazol-3-yl]pyridazin-3-one | 436.215 | 436.3 |
| 60 | 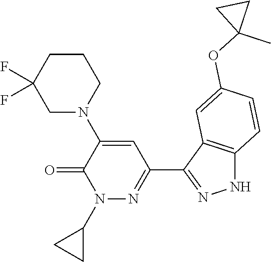 [SMILES: CC1(Oc2ccc3[nH]nc(-c4cc(N5CCCC(F)(F)C5)c(=O)n(C5CC5)n4)c3c2)CC1] | 2-cyclopropyl-4-(3,3-difluoro- 1-piperidyl)-6-[5-(1- methylcyclopropoxy)-1H- indazol-3-yl]pyridazin-3-one | 442.205 | 442.5 |
| 61 | 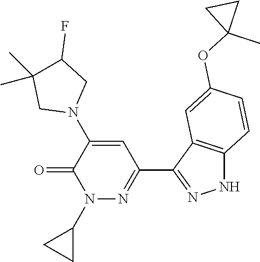 | 2-cyclopropyl-4-(4-fluoro-3,3- dimethyl-pyrrolidin-1-yl)-6-[5- (1-methylcyclopropoxy)-1H- indazol-3-yl]pyridazin-3-one | 438.231 | 438.3 |
| 62 | 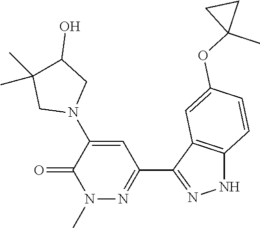 | 4-(4-hydroxy-3,3-dimethyl- pyrrolidin-1-yl)-2-methyl-6-[5- (1-methylcyclopropoxy)-1H- indazol-3-yl]pyridazin-3-one | 410.219 | 410.4 |
| 63 | 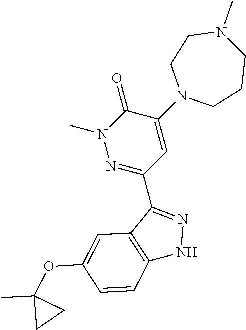 [SMILES: CN1CCCN(c2cc(-c3n[nH]c4ccc(OC5(C)CC5)cc34)nn(C)c2=O)CC1] | 2-methyl-6-[5-(1- methylcyclopropoxy)-1H- indazol-3-yl]-4-(4-methyl-1,4- diazepan-1-yl)pyridazin-3-one | 409.235 | 409.3 |
| 64 | 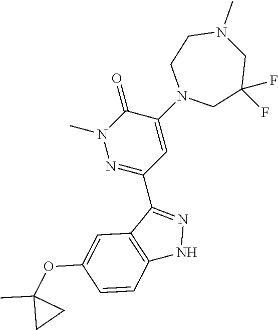 [SMILES: CN1CCN(c2cc(-c3n[nH]c4ccc(OC5(C)CC5)cc34)nn(C)c2=O)CC(F)(F)C1] | 4-(6,6-difluoro-4-methyl-1,4- diazepan-1-yl)-2-methyl-6-[5- (1-methylcyclopropoxy)-1H- indazol-3-yl]pyridazin-3-one | 445.216 | 445.4 |
| 65 | 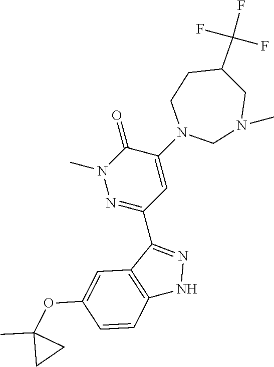 | 2-methyl-6-[5-(1- methylcyclopropoxy)-1H- indazol-3-yl]-4-[4-methyl-5- (trifluoromethyl)-1,4-diazepan- 1-yl]pyridazin-3-one | 477.223 | 477.3 |
| 66 | 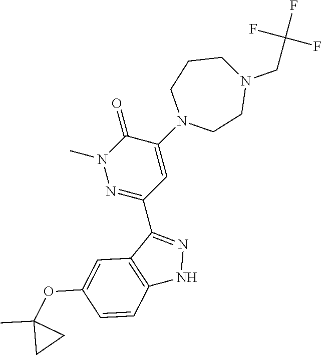 [SMILES: Cn1nc(-c2n[nH]c3ccc(OC4(C)CC4)cc23)cc(N2CCCN(CC(F)(F)F)CC2)c1=O] | 2-methyl-6-[5-(1- methylcyclopropoxy)-1H- indazol-3-yl]-4-[4-(2,2,2- trifluoroethyl)-1,4-diazepan-1- yl]pyridazin-3-one | 477.223 | 477.3 |
| 67 | 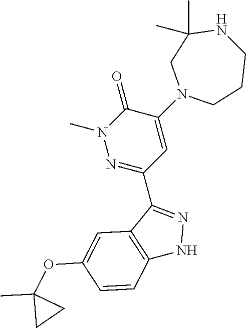 [SMILES: Cn1nc(-c2n[nH]c3ccc(OC4(C)CC4)cc23)cc(N2CCCNC(C)(C)C2)c1=O] | 4-(3,3-dimethyl-1,4-diazepan- 1-yl)-2-methyl-6-[5-(1- methylcyclopropoxy)-1H- indazol-3-yl]pyridazin-3-one | 423.251 | 423.5 |
| 68 | 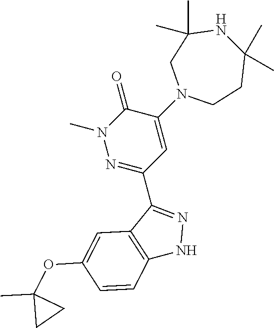 | 2-methyl-6-[5-(1- methylcyclopropoxy)-1H- indazol-3-yl]-4-(3,3,5,5- tetramethyl-1,4-diazepan-1- yl)pyridazin-3-one | 451.282 | 451.3 |
| 69 | 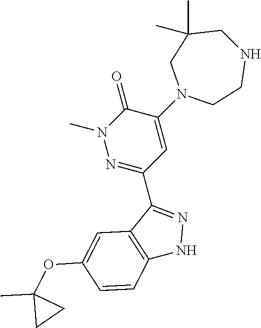 [SMILES: Cn1nc(-c2n[nH]c3ccc(OC4(C)CC4)cc23)cc(N2CCNCC(C)(C)C2)c1=O] | 4-(6,6-dimethyl-1,4-diazepan- 1-yl)-2-methyl-6-[5-(1- methylcyclopropoxy)-1H- indazol-3-yl]pyridazin-3-one | 423.251 | 423.5 |
| 70 | 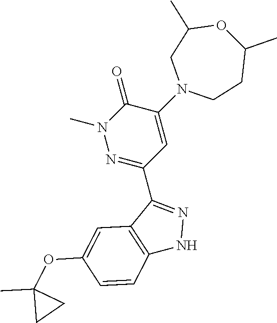 | 4-(2,7-dimethyl-1,4-oxazepan- 4-yl)-2-methyl-6-[5-(1- methylcyclopropoxy)-1H- indazol-3-yl]pyridazin-3-one | 424.235 | 424.3 |
| 71 | 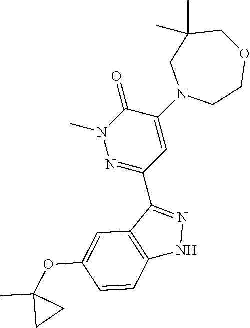 [SMILES: Cn1nc(-c2n[nH]c3ccc(OC4(C)CC4)cc23)cc(N2CCOCC(C)(C)C2)c1=O] | 4-(6,6-dimethyl-1,4-oxazepan- 4-yl)-2-methyl-6-[5-(1- methylcyclopropoxy)-1H- indazol-3-yl]pyridazin-3-one | 424.235 | 424.3 |
| 72 | 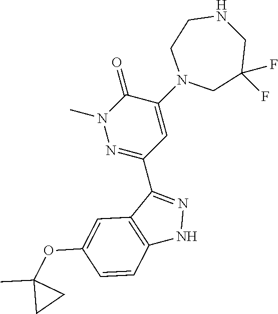 [SMILES: Cn1nc(-c2n[nH]c3ccc(OC4(C)CC4)cc23)cc(N2CCNCC(F)(F)C2)c1=O] | 4-(6,6-difluoro-1,4-diazepan- 1-yl)-2-methyl-6-[5-(1- methylcyclopropoxy)-1H- indazol-3-yl]pyridazin-3-one | 431.201 | 431.5 |
| 73 | 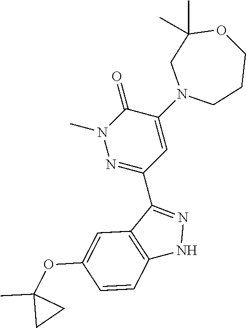 [SMILES: Cn1nc(-c2n[nH]c3ccc(OC4(C)CC4)cc23)cc(N2CCCOC(C)(C)C2)c1=O] | 4-(2,2-dimethyl-1,4-oxazepan- 4-yl)-2-methyl-6-[5-(1- methylcyclopropoxy)-1H- indazol-3-yl]pyridazin-3-one | 424.235 | 424.3 |
| 74 | 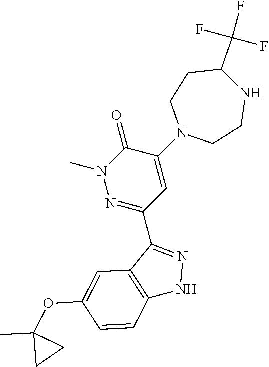 [SMILES: Cn1nc(-c2n[nH]c3ccc(OC4(C)CC4)cc23)cc(N2CCNC(C(F)(F)F)CC2)c1=O] | 2-methyl-6-[5-(1- methylcyclopropoxy)-1H- indazol-3-yl]-4-[5- (trifluoromethyl)-1,4-diazepan- 1-yl]pyridazin-3-one | 463.207 | 463.2 |
| 75 | 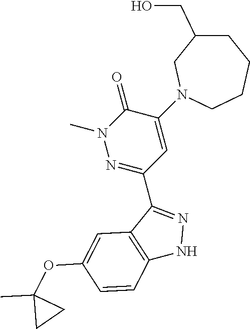 [SMILES: Cn1nc(-c2n[nH]c3ccc(OC4(C)CC4)cc23)cc(N2CCCCC(CO)C2)c1=O] | 4-[3-(hydroxymethyl)azepan- 1-yl]-2-methyl-6-[5-(1- methylcyclopropoxy)-1H- indazol-3-yl]pyridazin-3-one | 424.235 | 424.5 |
| 76 | 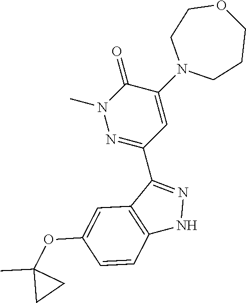 [SMILES: Cn1nc(-c2n[nH]c3ccc(OC4(C)CC4)cc23)cc(N2CCCOCC2)c1=O] | 2-methyl-6-[5-(1- methylcyclopropoxy)-1H- indazol-3-yl]-4-(1,4-oxazepan- 4-yl)pyridazin-3-one | 396.204 | 396.3 |
| 77 | 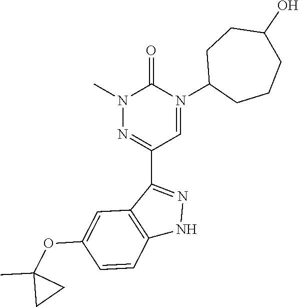 | 4-(4-hydroxyazepan-1-yl)-2- methyl-6-[5-(1- methylcyclopropoxy)-1H- indazol-3-yl]pyridazin-3-one | 410.219 | 410.3 |
| 78 | 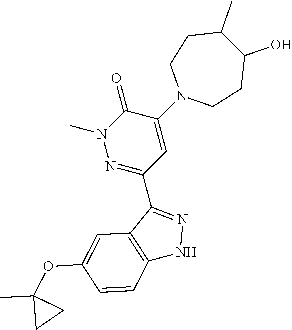 [SMILES: CC1CCN(c2cc(-c3n[nH]c4ccc(OC5(C)CC5)cc34)nn(C)c2=O)CCC1O] | 4-(4-hydroxy-5-methyl- azepan-1-yl)-2-methyl-6-[5-(1- methylcyclopropoxy)-1H- indazol-3-yl]pyridazin-3-one | 424.235 | 424.5 |
| 79 | 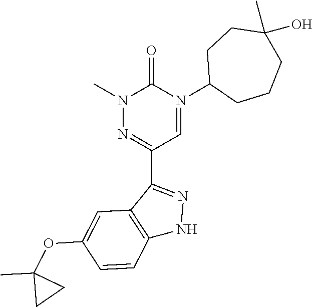 | 4-(4-hydroxy-4-methyl- azepan-1-yl)-2-methyl-6-[5-(1- methylcyclopropoxy)-1H- indazol-3-yl]pyridazin-3-one | 424.235 | 424.3 |
| 80 | 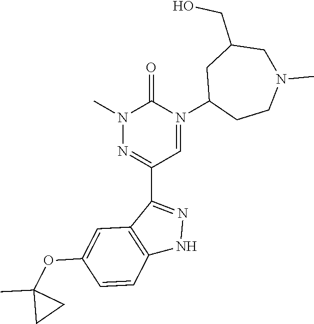 | 4-[6-(hydroxymethyl)-4- methyl-1,4-diazepan-1-yl]-2- methyl-6-[5-(1- methylcyclopropoxy)-1H- indazol-3-yl]pyridazin-3-one | 439.246 | 439.3 |
| 81 | 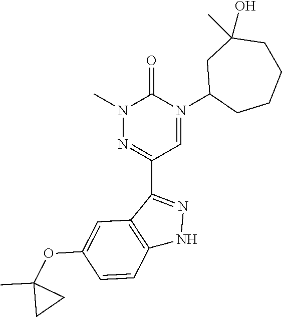 | 4-(3-hydroxy-3-methyl- azepan-1-yl)-2-methyl-6-[5-(1- methylcyclopropoxy)-1H- indazol-3-yl]pyridazin-3-one | 424.235 | 424.3 |
| 82 | 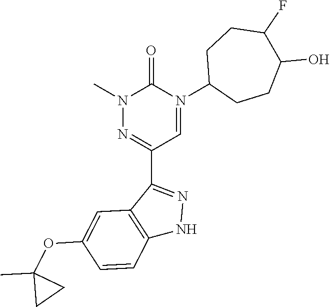 | 4-(4-fluoro-5-hydroxy-azepan- 1-yl)-2-methyl-6-[5-(1- methylcyclopropoxy)-1H- indazol-3-yl]pyridazin-3-one | 428.210 | 428.3 |
| 83 | 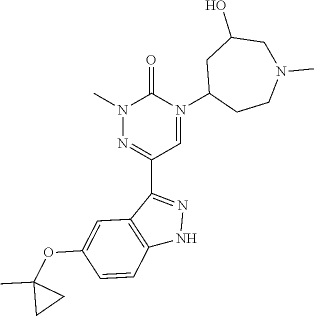 | 4-(6-hydroxy-4-methyl-1,4- diazepan-1-yl)-2-methyl-6-[5- (1-methylcyclopropoxy)-1H- indazol-3-yl]pyridazin-3-one | 425.230 | 425.3 |
| 84 | 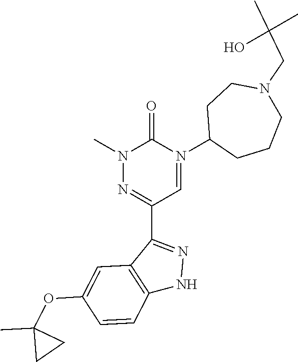 | 4-[4-(2-hydroxy-2-methyl- propyl)-1,4-diazepan-1-yl]-2- methyl-6-[5-(1- methylcyclopropoxy)-1H- indazol-3-yl]pyridazin-3-one | 467.277 | 467.5 |
| 85 | 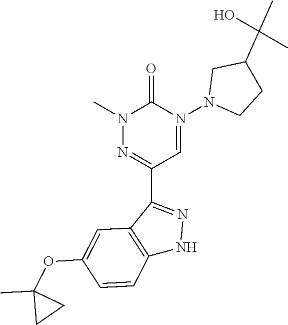 | 4-[3-(1-hydroxy-1-methyl- ethyl)pyrrolidin-1-yl]-2- methyl-6-[5-(1- methylcyclopropoxy)-1H- indazol-3-yl]pyridazin-3-one | 424.235 | 424.3 |
| 86 | 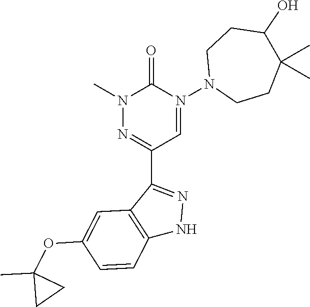 | 4-(5-hydroxy-4,4-dimethyl- azepan-1-yl)-2-methyl-6-[5-(1- methylcyclopropoxy)-1H- indazol-3-yl]pyridazin-3-one | 438.251 | 438.3 |
| 87 | 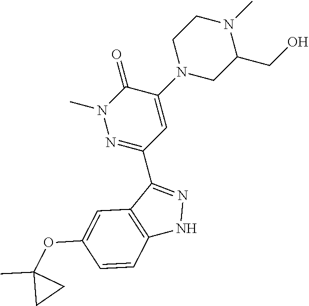 [SMILES: CN1CCN(c2cc(-c3n[nH]c4ccc(OC5(C)CC5)cc34)nn(C)c2=O)CC1CO] | 4-[3-(hydroxymethyl)-4- methyl-piperazin-1-yl]-2- methyl-6-[5-(1- methylcyclopropoxy)-1H- indazol-3-yl]pyridazin-3-one | 425.230 | 425.4 |
| 88 | 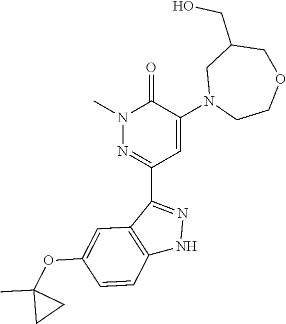 [SMILES: Cn1nc(-c2n[nH]c3ccc(OC4(C)CC4)cc23)cc(N2CCOCC(CO)C2)c1=O] | 4-[6-(hydroxymethyl)-1,4- oxazepan-4-yl]-2-methyl-6-[5- (1-methylcyclopropoxy)-1H- indazol-3-yl]pyridazin-3-one | 426.214 | 426.3 |
| 89 | 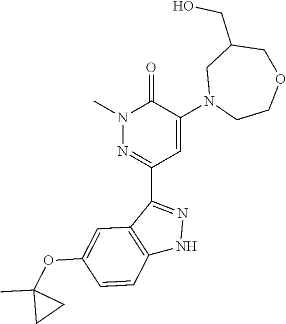 [SMILES: Cn1nc(-c2n[nH]c3ccc(OC4(C)CC4)cc23)cc(N2CCOCC(CO)C2)c1=O] | 4-[2-(hydroxymethyl)-1,4- oxazepan-4-yl]-2-methyl-6-[5- (1-methylcyclopropoxy)-1H- indazol-3-yl]pyridazin-3-one | 426.214 | 426.3 |
| 90 | 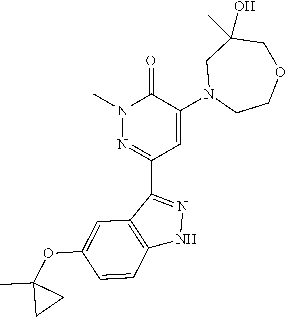 [SMILES: Cn1nc(-c2n[nH]c3ccc(OC4(C)CC4)cc23)cc(N2CCOCC(C)(O)C2)c1=O] | 4-(6-hydroxy-6-methyl-1,4- oxazepan-4-yl)-2-methyl-6-[5- (1-methylcyclopropoxy)-1H- indazol-3-yl]pyridazin-3-one | 426.214 | 426.6 |
| 91 | 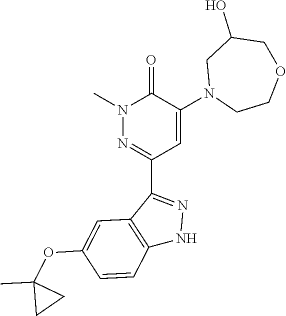 [SMILES: Cn1nc(-c2n[nH]c3ccc(OC4(C)CC4)cc23)cc(N2CCOCC(O)C2)c1=O] | 4-(6-hydroxy-1,4-oxazepan-4- yl)-2-methyl-6-[5-(1- methylcyclopropoxy)-1H- indazol-3-yl]pyridazin-3-one | 412.198 | 412.3 |
| 92 | 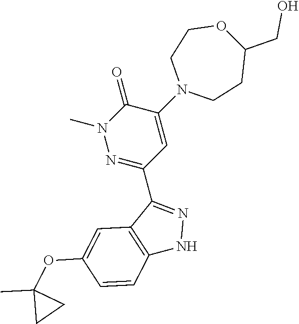 [SMILES: Cn1nc(-c2n[nH]c3ccc(OC4(C)CC4)cc23)cc(N2CCOC(CO)CC2)c1=O] | 4-[7-(hydroxymethyl)-1,4- oxazepan-4-yl]-2-methyl-6-[5- (1-methylcyclopropoxy)-1H- indazol-3-yl]pyridazin-3-one | 426.214 | 426.3 |
| 93 | 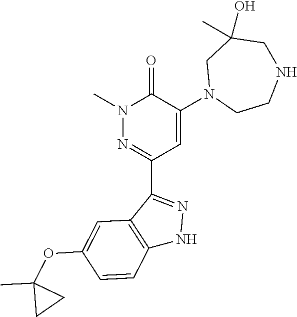 [SMILES: Cn1nc(-c2n[nH]c3ccc(OC4(C)CC4)cc23)cc(N2CCNCC(C)(O)C2)c1=O] | 4-(6-hydroxy-6-methyl-1,4- diazepan-1-yl)-2-methyl-6-[5- (1-methylcyclopropoxy)-1H- indazol-3-yl]pyridazin-3-one | 425.230 | 425.3 |
| 94 | 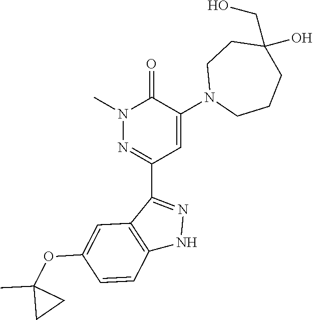 [SMILES: Cn1nc(-c2n[nH]c3ccc(OC4(C)CC4)cc23)cc(N2CCCC(O)(CO)CC2)c1=O] | 4-[4-hydroxy-4- (hydroxymethyl)azepan-1-yl]- 2-methyl-6-[5-(1- methylcyclopropoxy)-1H- indazol-3-yl]pyridazin-3-one | 440.230 | 440.6 |
| 95 | 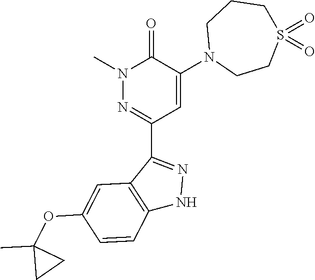 [SMILES: Cn1nc(-c2n[nH]c3ccc(OC4(C)CC4)cc23)cc(N2CCCS(=O)(=O)CC2)c1=O] | 4-(1,1-dioxo-1,4-thiazepan-4- yl)-2-methyl-6-[5-(1- methylcyclopropoxy)-1H- indazol-3-yl]pyridazin-3-one | 444.171 | 444.2 |
| 96 | 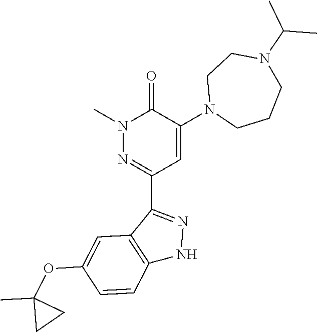 [SMILES: CC(C)N1CCCN(c2cc(-c3n[nH]c4ccc(OC5(C)CC5)cc34)nn(C)c2=O)CC1] | 4-(4-isopropyl-1,4-diazepan-1- yl)-2-methyl-6-[5-(1- methylcyclopropoxy)-1H- indazol-3-yl]pyridazin-3-one | 437.267 | 437.3 |
| 97 | 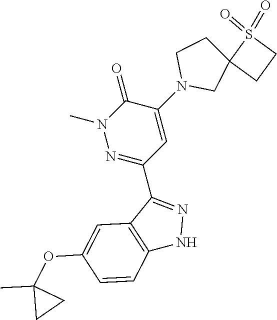 [SMILES: Cn1nc(-c2n[nH]c3ccc(OC4(C)CC4)cc23)cc(N2CCC3(CCS3(=O)=O)C2)c1=O] | 4-(1,1-dioxo-1lambda6-thia-7- azaspiro[3.4]octan-7-yl)-2- methyl-6-[5-(1- methylcyclopropoxy)-1H- indazol-3-yl]pyridazin-3-one | 456.171 | 456.2 |
| 98 | 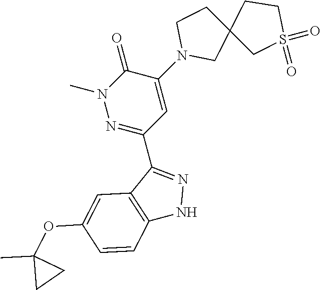 [SMILES: Cn1nc(-c2n[nH]c3ccc(OC4(C)CC4)cc23)cc(N2CCC3(CCS(=O)(=O)C3)C2)c1=O] | 4-(2,2-dioxo-2lambda6-thia-7- azaspiro[4.4]nonan-7-yl)-2- methyl-6-[5-(1- methylcyclopropoxy)-1H- indazol-3-yl]pyridazin-3-one | 470.186 | 470.2 |
| 99 | 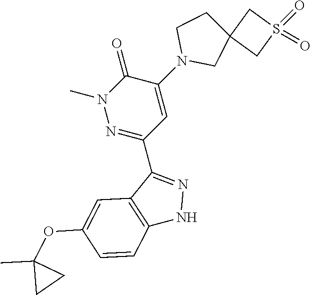 [SMILES: Cn1nc(-c2n[nH]c3ccc(OC4(C)CC4)cc23)cc(N2CCC3(C2)CS(=O)(=O)C3)c1=O] | 4-(2,2-dioxo-2lambda6-thia-7- azaspiro[3.4]octan-7-yl)-2- methyl-6-[5-(1- methylcyclopropoxy)-1H- indazol-3-yl]pyridazin-3-one | 456.171 | 456.3 |
| 100 | 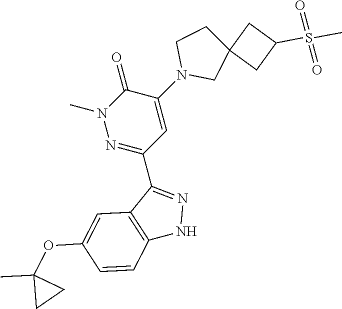 | 2-methyl-6-[5-(1- methylcyclopropoxy)-1H- indazol-3-yl]-4-(2- methylsulfonyl-6- azaspiro[3.4]octan-6- yl)pyridazin-3-one | 484.202 | 484.4 |
| 101 | 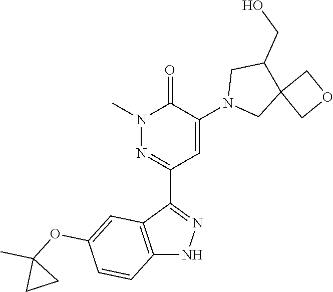 [SMILES: Cn1nc(-c2n[nH]c3ccc(OC4(C)CC4)cc23)cc(N2CC(CO)C3(COC3)C2)c1=O] | 4-[5-(hydroxymethyl)-2-oxa-7- azaspiro[3.4]octan-7-yl]-2- methyl-6-[5-(1- methylcyclopropoxy)-1H- indazol-3-yl]pyridazin-3-one | 438.214 | 438.3 |
| 102 | 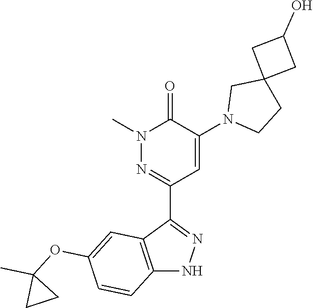 [SMILES: Cn1nc(-c2n[nH]c3ccc(OC4(C)CC4)cc23)cc(N2CCC3(CC(O)C3)C2)c1=O] | 4-(2-hydroxy-6- azaspiro[3.4]octan-6-yl)-2- methyl-6-[5-(1- methylcyclopropoxy)-1H- indazol-3-yl]pyridazin-3-one | 422.219 | 422.4 |
| 103 | 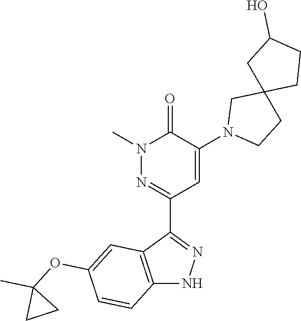 [SMILES: Cn1nc(-c2n[nH]c3ccc(OC4(C)CC4)cc23)cc(N2CCC3(CCC(O)C3)C2)c1=O] | 4-(8-hydroxy-2- azaspiro[4.4]nonan-2-yl)-2- methyl-6-[5-(1- methylcyclopropoxy)-1H- indazol-3-yl]pyridazin-3-one | 436.235 | 436.5 |
| 104 | 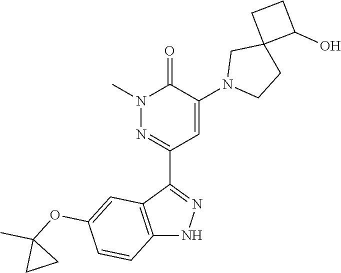 [SMILES: Cn1nc(-c2n[nH]c3ccc(OC4(C)CC4)cc23)cc(N2CCC3(CCC3O)C2)c1=O] | 4-(3-hydroxy-6- azaspiro[3.4]octan-6-yl)-2- methyl-6-[5-(1- methylcyclopropoxy)-1H- indazol-3-yl]pyridazin-3-one | 422.219 | 422.3 |
| 105 | 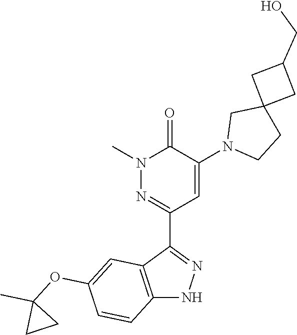 [SMILES: Cn1nc(-c2n[nH]c3ccc(OC4(C)CC4)cc23)cc(N2CCC3(CC(CO)C3)C2)c1=O] | 4-[2-(hydroxymethyl)-6- azaspiro[3.4]octan-6-yl]-2- methyl-6-[5-(1- methylcyclopropoxy)-1H- indazol-3-yl]pyridazin-3-one | 436.235 | 436.6 |
| 106 | 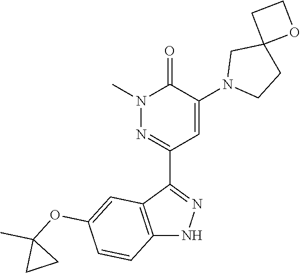 [SMILES: Cn1nc(-c2n[nH]c3ccc(OC4(C)CC4)cc23)cc(N2CCC3(CCO3)C2)c1=O] | 2-methyl-6-[5-(1- methylcyclopropoxy)-1H- indazol-3-yl]-4-(1-oxa-7- azaspiro[3.4]octan-7- yl)pyridazin-3-one | 408.204 | 408.5 |
| 107 | 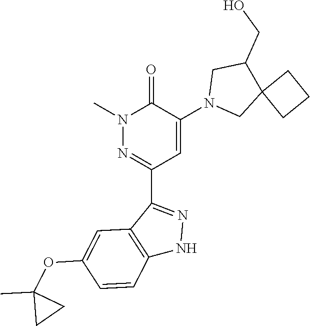 [SMILES: Cn1nc(-c2n[nH]c3ccc(OC4(C)CC4)cc23)cc(N2CC(CO)C3(CCC3)C2)c1=O] | 4-[8-(hydroxymethyl)-6- azaspiro[3.4]octan-6-yl]-2- methyl-6-[5-(1- methylcyclopropoxy)-1H- indazol-3-yl]pyridazin-3-one | 436.235 | 436.3 |
| 108 | 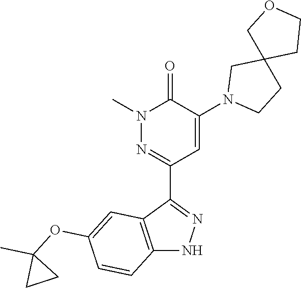 [SMILES: Cn1nc(-c2n[nH]c3ccc(OC4(C)CC4)cc23)cc(N2CCC3(CCOC3)C2)c1=O] | 2-methyl-6-[5-(1- methylcyclopropoxy)-1H- indazol-3-yl]-4-(2-oxa-7- azaspiro[4.4]nonan-7- yl)pyridazin-3-one | 422.219 | 422.5 |
| 109 | 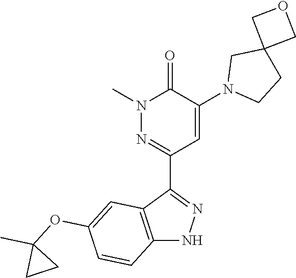 [SMILES: Cn1nc(-c2n[nH]c3ccc(OC4(C)CC4)cc23)cc(N2CCC3(COC3)C2)c1=O] | 2-methyl-6-[5-(1- methylcyclopropoxy)-1H- indazol-3-yl]-4-(2-oxa-7- azaspiro[3.4]octan-7- yl)pyridazin-3-one | 408.204 | 408.3 |
| 110 | 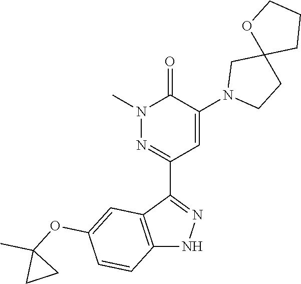 [SMILES: Cn1nc(-c2n[nH]c3ccc(OC4(C)CC4)cc23)cc(N2CCC3(CCCO3)C2)c1=O] | 2-methyl-6-[5-(1- methylcyclopropoxy)-1H- indazol-3-yl]-4-(1-oxa-7- azaspiro[4.4]nonan-7- yl)pyridazin-3-one | 422.219 | 422.3 |
| 111 | 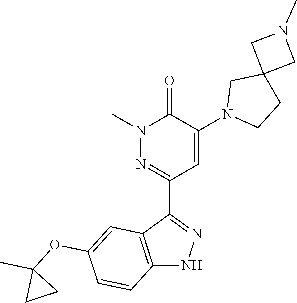 | 2-methyl-6-[5-(1- methylcyclopropoxy)-1H- indazol-3-yl]-4-(2-methyl-2,6- diazaspiro[3.4]octan-6- yl)pyridazin-3-one | 421.235 | 421.3 |
| 112 | 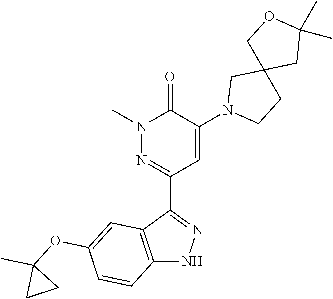 | 4-(3,3-dimethyl-2-oxa-7- azaspiro[4.4]nonan-7-yl)-2- methyl-6-[5-(1- methylcyclopropoxy)-1H- indazol-3-yl]pyridazin-3-one | 450.251 | 450.4 |
| 113 | 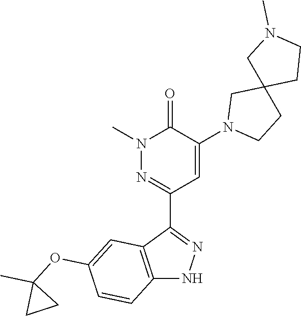 [SMILES: CN1CCC2(CCN(c3cc(-c4n[nH]c5ccc(OC6(C)CC6)cc45)nn(C)c3=O)C2)C1] | 2-methyl-6-[5-(1- methylcyclopropoxy)-1H- indazol-3-yl]-4-(7-methyl-2,7- diazaspiro[4.4]nonan-2- yl)pyridazin-3-one | 435.251 | 435.4 |
| 114 | 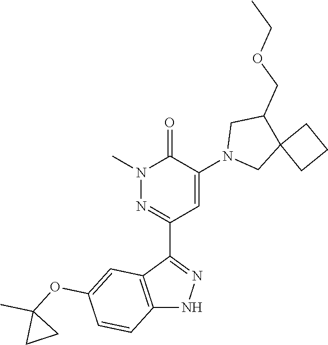 [SMILES: CCOCC1CN(c2cc(-c3n[nH]c4ccc(OC5(C)CC5)cc34)nn(C)c2=O)CC12CCC2] | 4-[8-(ethoxymethyl)-6- azaspiro[3.4]octan-6-yl]-2- methyl-6-[5-(1- methylcyclopropoxy)-1H- indazol-3-yl]pyridazin-3-one | 464.266 | 464.3 |
| 115 | 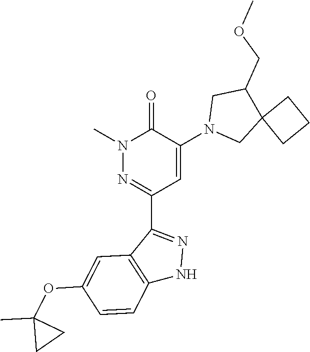 [SMILES: COCC1CN(c2cc(-c3n[nH]c4ccc(OC5(C)CC5)cc34)nn(C)c2=O)CC12CCC2] | 4-[8-(methoxymethyl)-6- azaspiro[3.4]octan-6-yl]-2- methyl-6-[5-(1- methylcyclopropoxy)-1H- indazol-3-yl]pyridazin-3-one | 450.251 | 450.3 |
| 116 | 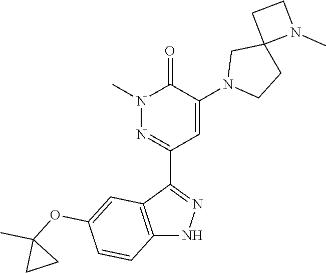 | 2-methyl-6-[5-(1- methylcyclopropoxy)-1H- indazol-3-yl]-4-(1-methyl-1,7- diazaspiro[3.4]octan-7- yl)pyridazin-3-one | 421.235 | 421.4 |
| 117 | 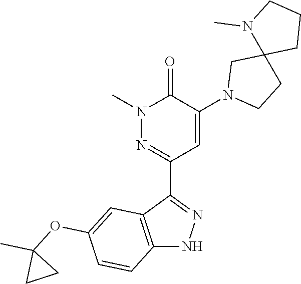 [SMILES: CN1CCCC12CCN(c1cc(-c3n[nH]c4ccc(OC5(C)CC5)cc34)nn(C)c1=O)C2] | 2-methyl-6-[5-(1- methylcyclopropoxy)-1H- indazol-3-yl]-4-(1-methyl-1,7- diazaspiro[4.4]nonan-7- yl)pyridazin-3-one | 435.251 | 435.3 |
| 118 | 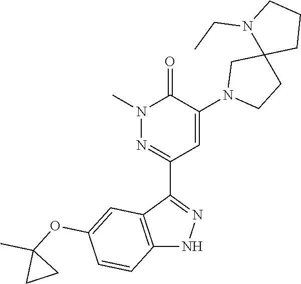 [SMILES: CCN1CCCC12CCN(c1cc(-c3n[nH]c4ccc(OC5(C)CC5)cc34)nn(C)c1=O)C2] | 4-(1-ethyl-1,7- diazaspiro[4.4]nonan-7-yl)-2- methyl-6-[5-(1- methylcyclopropoxy)-1H- indazol-3-yl]pyridazin-3-one | 449.267 | 449.5 |
| 119 | 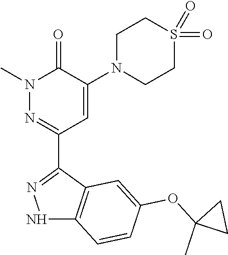 [SMILES: Cn1nc(-c2n[nH]c3ccc(OC4(C)CC4)cc23)cc(N2CCS(=O)(=O)CC2)c1=O] | 4-(1,1-dioxo-1,4-thiazinan-4- yl)-2-methyl-6-[5-(1- methylcyclopropoxy)-1H- indazol-3-yl]pyridazin-3-one | 430.155 | 430.5 |
| 120 | 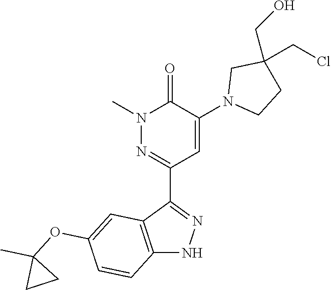 [SMILES: Cn1nc(-c2n[nH]c3ccc(OC4(C)CC4)cc23)cc(N2CCC(CO)(CCl)C2)c1=O] | 4-[3-(chloromethyl)-3- (hydroxymethyl)pyrrolidin-1- yl]-2-methyl-6-[5-(1- methylcyclopropoxy)-1H- indazol-3-yl]pyridazin-3-one | 444.180 | 444.4 |
| 121 | 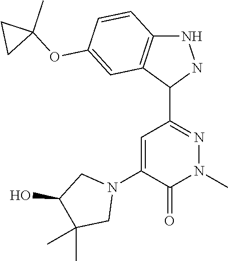 [SMILES: Cn1nc(C2NNc3ccc(OC4(C)CC4)cc32)cc(N2C[C@H](O)C(C)(C)C2)c1=O] | 4-[(4R)-4-hydroxy-3,3- dimethyl-pyrrolidin-1-yl]-2- methyl-6-[5-(1- methylcyclopropoxy)-1H- indazol-3-yl]pyridazin-3-one | 410.219 | 410.4 |
| 122 | 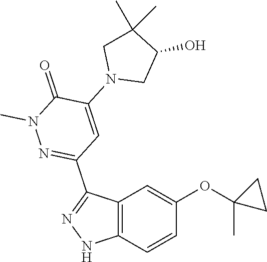 [SMILES: Cn1nc(-c2n[nH]c3ccc(OC4(C)CC4)cc23)cc(N2C[C@@H](O)C(C)(C)C2)c1=O] | 4-[(4S)-4-hydroxy-3,3- dimethyl-pyrrolidin-1-yl]-2- methyl-6-[5-(1- methylcyclopropoxy)-1H- indazol-3-yl]pyridazin-3-one | 410.219 | 410.3 |
| 123 | 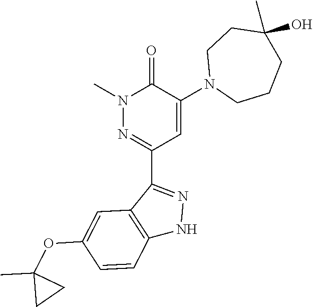 [SMILES: Cn1nc(-c2n[nH]c3ccc(OC4(C)CC4)cc23)cc(N2CCC[C@](C)(O)CC2)c1=O] | 4-[(4S)-4-hydroxy-4-methyl- azepan-1-yl]-2-methyl-6-[5-(1- methylcyclopropoxy)-1H- indazol-3-yl]pyridazin-3-one | 424.235 | 424.6 |
| 124 | 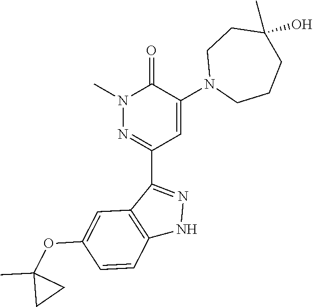 [SMILES: Cn1nc(-c2n[nH]c3ccc(OC4(C)CC4)cc23)cc(N2CCC[C@@](C)(O)CC2)c1=O] | 4-[(4R)-4-hydroxy-4-methyl- azepan-1-yl]-2-methyl-6-[5-(1- methylcyclopropoxy)-1H- indazol-3-yl]pyridazin-3-one | 424.235 | 424.6 |
| 125 | 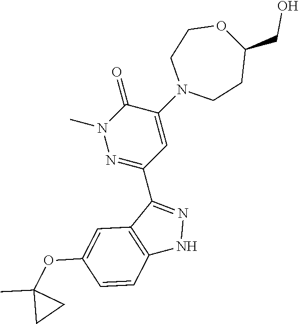 [SMILES: Cn1nc(-c2n[nH]c3ccc(OC4(C)CC4)cc23)cc(N2CCO[C@@H](CO)CC2)c1=O] | 4-[(7R)-7-(hydroxymethyl)- 1,4-oxazepan-4-yl]-2-methyl- 6-[5-(1-methylcyclopropoxy)- 1H-indazol-3-yl]pyridazin-3- one | 426.214 | 426.5 |
| 126 | 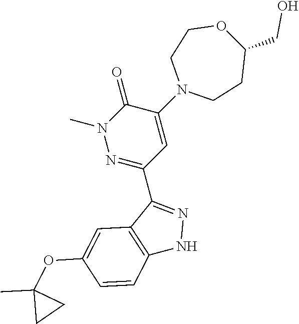 [SMILES: Cn1nc(-c2n[nH]c3ccc(OC4(C)CC4)cc23)cc(N2CCO[C@H](CO)CC2)c1=O] | 4-[(7S)-7-(hydroxymethyl)- 1,4-oxazepan-4-yl]-2-methyl- 6-[5-(1-methylcyclopropoxy)- 1H-indazol-3-yl]pyridazin-3- one | 426.214 | 426.3 |
| 127 | 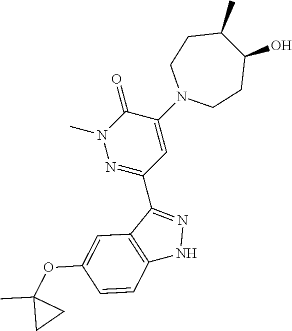 [SMILES: C[C@@H]1CCN(c2cc(-c3n[nH]c4ccc(OC5(C)CC5)cc34)nn(C)c2=O)CC[C@@H]1O] | 4-[(4S,5R)-4-hydroxy-5- methyl-azepan-1-yl]-2-methyl- 6-[5-(1-methylcyclopropoxy)- 1H-indazol-3-yl]pyridazin-3- one | 424.235 | 424.6 |
| 128 | 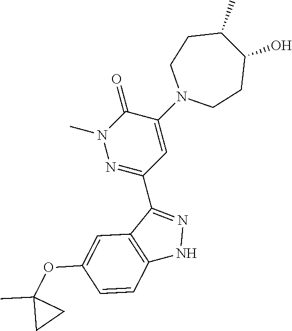 [SMILES: C[C@H]1CCN(c2cc(-c3n[nH]c4ccc(OC5(C)CC5)cc34)nn(C)c2=O)CC[C@H]1O] | 4-[(4R,5S)-4-hydroxy-5- methyl-azepan-1-yl]-2-methyl- 6-[5-(1-methylcyclopropoxy)- 1H-indazol-3-yl]pyridazin-3- one | 424.235 | 424.7 |
| 129 | 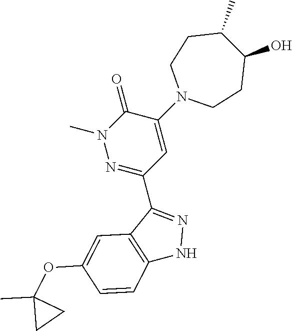 [SMILES: C[C@H]1CCN(c2cc(-c3n[nH]c4ccc(OC5(C)CC5)cc34)nn(C)c2=O)CC[C@@H]1O] | 4-[(4S,5S)-4-hydroxy-5- methyl-azepan-1-yl]-2-methyl- 6-[5-(1-methylcyclopropoxy)- 1H-indazol-3-yl]pyridazin-3- one | 424.235 | 424.5 |
| 130 | 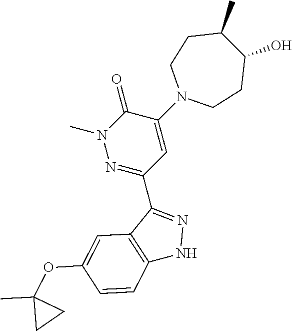 [SMILES: C[C@@H]1CCN(c2cc(-c3n[nH]c4ccc(OC5(C)CC5)cc34)nn(C)c2=O)CC[C@H]1O] | 4-[(4R,5R)-4-hydroxy-5- methyl-azepan-1-yl]-2-methyl- 6-[5-(1-methylcyclopropoxy)- 1H-indazol-3-yl]pyridazin-3- one | 424.235 | 424.5 |
| 131 | 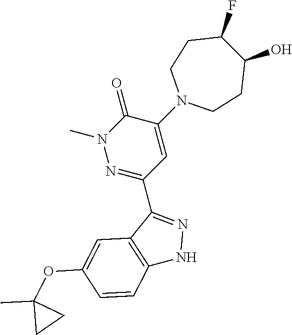 [SMILES: Cn1nc(-c2n[nH]c3ccc(OC4(C)CC4)cc23)cc(N2CC[C@@H](F)[C@@H](O)CC2)c1=O] | 4-[(4R,5S)-4-fluoro-5- hydroxy-azepan-1-yl]-2- methyl-6-[5-(1- methylcyclopropoxy)-1H- indazol-3-yl]pyridazin-3-one | 428.210 | 428.7 |
| 132 | 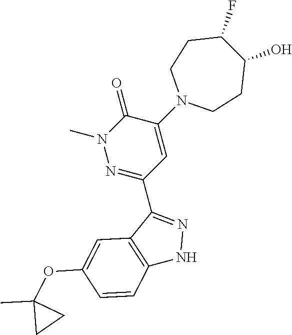 [SMILES: Cn1nc(-c2n[nH]c3ccc(OC4(C)CC4)cc23)cc(N2CC[C@@H](O)[C@@H](F)CC2)c1=O] | 4-[(4S,5R)-4-fluoro-5- hydroxy-azepan-1-yl]-2- methyl-6-[5-(1- methylcyclopropoxy)-1H- indazol-3-yl]pyridazin-3-one | 428.210 | 428.6 |
| 133 | 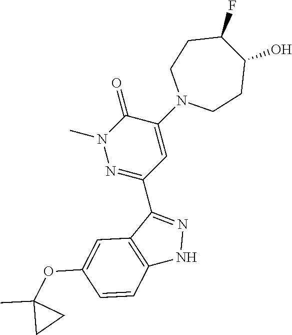 [SMILES: Cn1nc(-c2n[nH]c3ccc(OC4(C)CC4)cc23)cc(N2CC[C@@H](O)[C@H](F)CC2)c1=O] | 4-[(4R,5R)-4-fluoro-5- hydroxy-azepan-1-yl]-2- methyl-6-[5-(1- methylcyclopropoxy)-1H- indazol-3-yl]pyridazin-3-one | 428.210 | 428.5 |
| 134 | 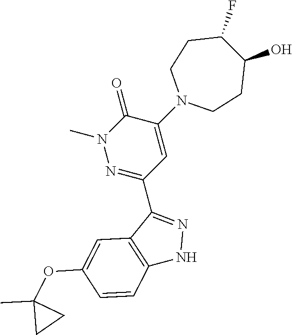 [SMILES: Cn1nc(-c2n[nH]c3ccc(OC4(C)CC4)cc23)cc(N2CC[C@H](O)[C@@H](F)CC2)c1=O] | 4-[(4S,5S)-4-fluoro-5- hydroxy-azepan-1-yl]-2- methyl-6-[5-(1- methylcyclopropoxy)-1H- indazol-3-yl]pyridazin-3-one | 428.210 | 428.6 |

### Synthesis of the Representative Examples of the Compound

### Example 1. 1-[5-(5-isopropoxy-1H-indazol-3-yl)pyridazin-3-yl]azetidin-3-ol (Compound 1)

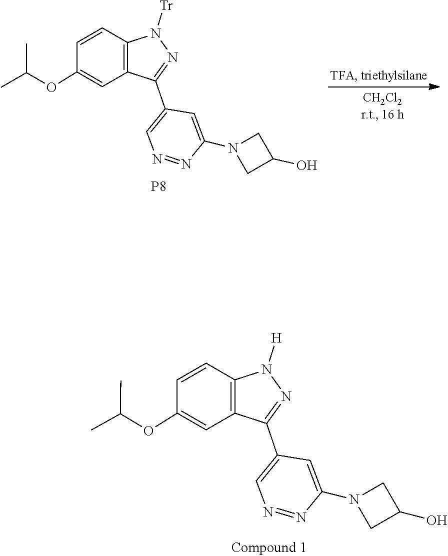

To a solution of P8 (47.0 mg, 0.083 mmol, 1.0 eq) in DCM (0.6 mL) was added TFA (0.6 mL) and triethylsilane (0.014 mL, 0.087 mmol, 1.05 eq). After the reaction mixture was stirred at rt for 16 h, the pH value of the mixture was adjusted to 7 by addition of saturated sodium bicarbonate aqueous solution. The solution was extracted with DCM for three times. The combined organic layers were dried over Na₂SO₄₍ₛ₎, filtered, and concentrated in vacuo. The residue was purified by C-18 reversed-phase column chromatography (mobile phase A: water (with NH₄HCO₃), mobile phase B: methanol, UV: 214 and 254 nm, Flow rate: 35 mL/min, Gradient: 3-90% (% B)) to give Compound 1 (13.0 mg, 48% yield) as a light-yellow solid. ¹H NMR (400 MHz, DMSO-d₆), δ: 13.54 (br. s, 1H), 9.13 (d, J=2.0 Hz, 1H), 7.56 (d, J=8.8 Hz, 1H), 7.46 (d, J=2.0 Hz, 1H), 7.11-7.08 (m, 2H), 5.76 (d, J=6.4 Hz, 1H), 4.76 (septet, J=6.0 Hz, 1H), 4.70-4.64 (m, 1H), 4.38-4.33 (m, 2H), 3.90-3.86 (m, 2H), 1.31 (d, J=6.0 Hz, 6H); LCMS (ESI) m z: calc. for C₁₇H₁₉N₅O₂ 325.15; found, 326.25 [M+H]⁺; HPLC purity: 97.5%, t_{R}=12.977 min.

### Example 2. 1-[5-(5-isopropoxy-1H-indazol-3-yl)pyridazin-3-yl]pyrrolidin-3-ol (Compound 2)

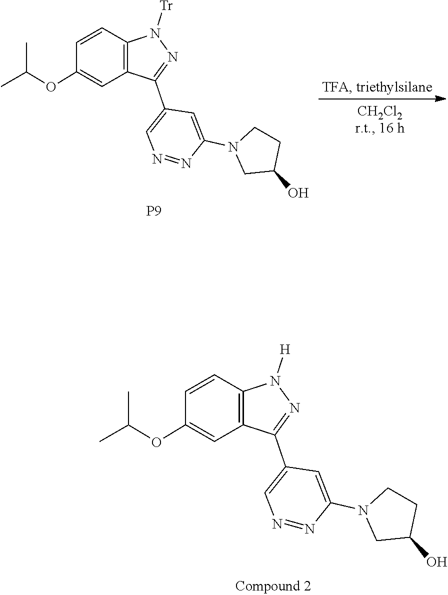

To a solution of P9 (112.0 mg, 0.193 mmol, 1.0 eq) in DCM (1.3 mL) was added TFA (1.3 mL) and triethylsilane (0.032 mL, 0.20 mmol, 1.05 eq). After the reaction mixture was stirred at rt for 16 h, the pH value was adjusted to ˜7 by addition of saturated sodium bicarbonate aqueous solution. The solution was extracted with DCM for three times. The combined organic layers were dried over Na₂SO₄₍ₛ₎, filtered, and concentrated in vacuo. The residue was purified by C-18 reversed-phase column chromatography (mobile phase A: water (with NH₄HCO₃), mobile phase B: methanol, UV: 214 and 254 nm, Flow rate: 35 mL/min, Gradient: 3-90% (% B)) to give Compound 2 (31.6 mg, 48% yield) as a white solid. ¹H NMR (400 MHz, DMSO-d₆), δ: 13.51 (br. s, 1H), 9.05 (d, J=1.6 Hz, 1H), 7.56 (d, J=9.0 Hz, 1H), 7.46 (d, J=2.0 Hz, 1H), 7.14 (d, J=1.6 Hz, 1H), 7.09 (d, J=9.0, 2.0 Hz, 1H), 5.05 (d, J=2.8 Hz, 1H), 4.74 (septet, J=6.0 Hz, 1H), 4.46 (br. m, 1H), 3.69-3.57 (m, 3H), 3.53-3.46 (m, 1H), 2.13-2.03 (m, 1H), 2.00-1.93 (m, 1H), 1.31 (d, J=6.0 Hz, 6H); LCMS (ESI) m z: calc. for C₁₈H₂₁N₅O₂ 339.17; found, 340.31 [M+H]⁺; HPLC purity: 98.8%, t_{R}=12.798 min.

### Example 3. 5-Isopropoxy-3-[6-(3-methylpyrrolidin-1-yl)pyridazin-4-yl]-1H-indazole (Compound 3)

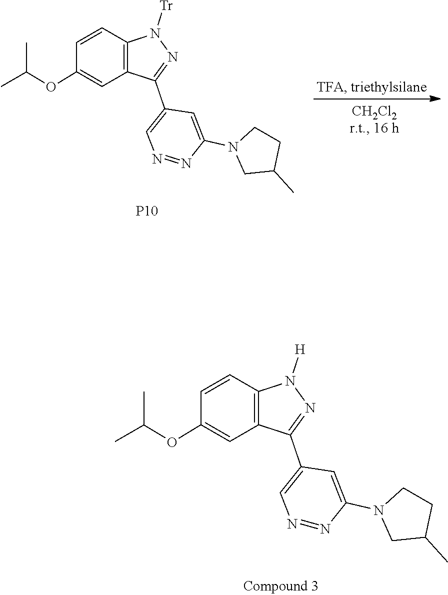

To a solution of P10 (66.0 mg, 0.114 mmol, 1.0 eq) in DCM (0.80 mL) was added TFA (0.80 mL) and triethylsilane (0.019 mL, 0.12 mmol, 1.05 eq). The reaction mixture was stirred at rt for 16 h. The pH value of the mixture was adjusted to ˜7 by addition of saturated sodium bicarbonate aqueous solution and extracted with DCM for three times. The combined organic layers were dried over Na₂SO₄₍ₛ₎, filtered, and concentrated in vacuo. The residue was purified by C-18 reversed-phase column chromatography (mobile phase A: water (with NH₄HCO₃), mobile phase B: methanol, UV: 214 and 254 nm, Flow rate: 35 mL/min, Gradient: 3-90% (% B)) to give Compound 3 (17.9 mg, 47% yield) as a white solid. ¹H NMR (400 MHz, CDCl₃), δ: 10.68 (br. s, 1H), 9.14 (d, J=2.2 Hz, 1H), 7.48 (d, J=9.2 Hz, 1H), 7.39 (d, J=2.2 Hz, 1H), 7.14-7.11 (m, 2H), 4.60 (septet, J=6.0 Hz, 1H), 3.87-3.74 (m, 2H), 3.64-3.56 (m, 1H), 3.21-3.15 (m, 1H), 2.51-2.42 (m, 1H), 2.25-2.17 (m, 1H), 1.76-1.68 (m, 1H), 1.39 (d, J=6.0 Hz, 6H), 1.18 (d, J=6.8 Hz, 3H); LCMS (ESI) m z: calc. for C₁₉H₂₃N₅O 337.19; found, 338.28 [M+H]⁺; HPLC purity: 99.4%, t_{R}=16.770 min.

### Example 4. 4-[5-(5-isopropoxy-1H-indazol-3-yl)pyridazin-3-yl]morpholine (Compound 4)

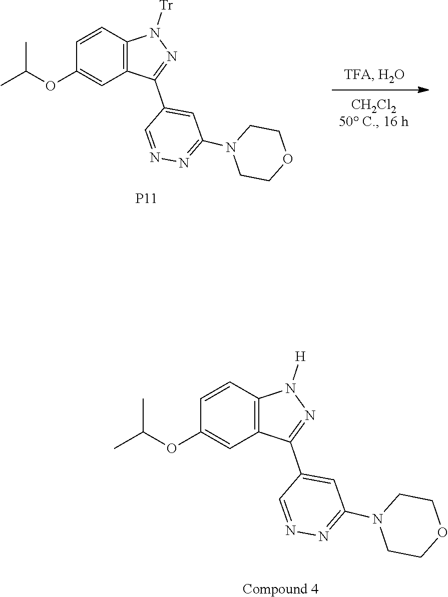

To a solution of P11 (40.0 mg, 0.0690 mmol, 1.0 eq) in DCM (0.50 mL) was added TFA (0.50 mL) and water (0.30 mL). The reaction mixture was stirred at 50° C. for 16 h. The pH value of the solution was adjusted to ˜7 by addition of saturated sodium bicarbonate aqueous solution and extracted with DCM for three times. The combined organic layers were dried over Na₂SO₄₍ₛ₎, filtered, and concentrated in vacuo. The residue was purified by C-18 reversed-phase column chromatography (mobile phase A: water (with NH₄HCO₃), mobile phase B: methanol, UV: 214 and 254 nm, Flow rate: 35 mL/min, Gradient: 3-90% (% B)) to give Compound 4 (10.6 mg, 45% yield) as a white solid. ¹H NMR (400 MHz, CDCl₃), δ: 10.45 (br. s, 1H), 9.25 (d, J=2.0 Hz, 1H), 7.48 (d, J=9.2 Hz, 1H), 7.43 (d, J=2.0 Hz, 1H), 7.38 (d, J=2.0 Hz, 1H), 7.15 (dd, J=9.2, 2.4 Hz, 1H), 4.61 (septet J=6.4 Hz, 1H), 3.91-3.88 (m, 4H), 3.76-3.72 (m, 4H), 1.39 (d, J=6.4 Hz, 6H); LCMS (ESI) m/z: 342.24 [M+H]⁺; HPLC purity: 99.8%, t_{R}=15.959 min.

### Example 5. 5-Isopropoxy-3-(6-pyrrolidin-1-ylpyridazin-4-yl)-1H-indazole (Compound 5)

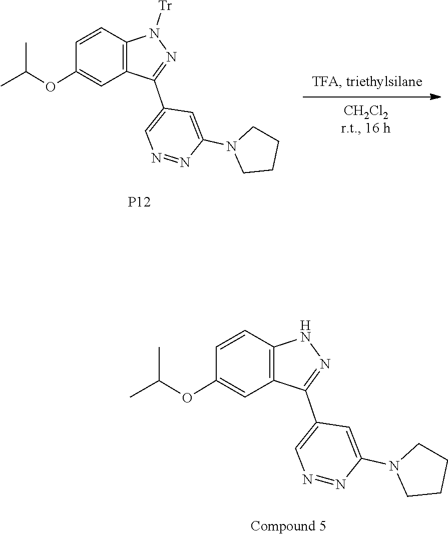

To a solution of P12 (200 mg, 0.354 mmol, 1.0 eq) in DCM (5.0 mL) was added TFA (1.0 mL) and triethylsilane (0.056 mL, 0.354 mmol, 1.0 eq). The reaction mixture was stirred at rt for 16 h. The pH value of the solution was adjusted to ˜7 by addition of saturated sodium bicarbonate aqueous solution and extracted with DCM for three times. The combined organic layers were dried over Na₂SO₄₍ₛ₎, filtered, and concentrated in vacuo. The residue was purified by C-18 reversed-phase column chromatography (mobile phase A: water (with NH₄HCO₃), mobile phase B: methanol, UV: 214 and 254 nm, Flow rate: 35 mL/min, Gradient: 3-90% (% B)) to give Compound 5 (60 mg, 53% yield) as a white solid. ¹H NMR (400 MHz, CDCl₃), δ: 10.39 (s, 1H), 9.14 (d, J=2.0 Hz, 1H), 7.47 (d, J=9.2 Hz, 1H), 7.39 (d, J=2.4 Hz, 1H), 7.16-7.11 (m, 2H), 4.60 (septet, J=6.0 Hz, 1H), 3.66-3.63 (m, J=6.8 Hz, 4H), 2.17-2.02 (m, 4H), 1.39 (d, J=6.0 Hz, 6H); LCMS (ESI) m z: calc. for C₁₈H₂₁N₅O 323.17; found, 324.2 [M+H]⁺; HPLC purity: 98.9%, t_{R}=15.298 min.

### Example 6. 4-(6-(5-isopropoxy-1H-indazol-3-yl)-3-methylpyridazin-4-yl)morpholine (Compound 10)

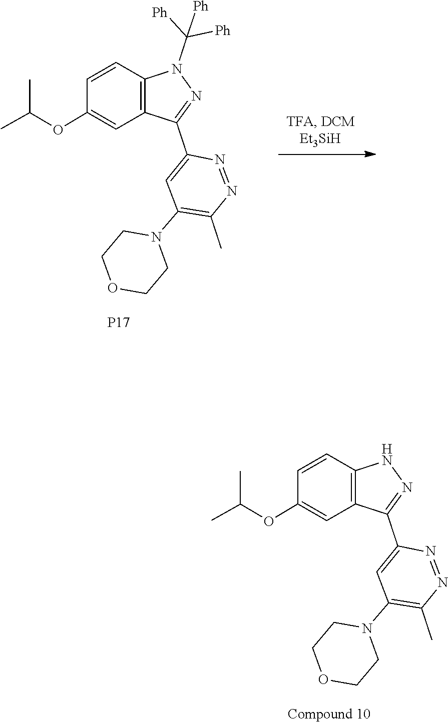

To a solution of P17 (40 mg, 0.067 mmol) in DCM (1.0 mL) was added TFA (1.0 mL) and triethylsilane (8.0 mg, 0.07 mmol) at rt. After the solution was stirred for overnight, the mixture was concentrated and then purified by C18 column (MeOH:water=5-100%) to give Compound 10 (10 mg, 42% yield). ¹H NMR (400 MHz, CD₃OD), δ: 8.03 (d, J=2.4 Hz, 1H), 7.82 (s, 1H), 7.47 (d, J=9.0 Hz, 1H), 7.08 (dd, J=9.0, 2.4 Hz, 1H), 4.68-4.65 (m, 1H), 3.91-3.87 (m, 4H), 3.21-3.19 (m, 4H), 2.69 (s, 3H), 1.36 (d, J=6.0 Hz, 6H); LCMS (ESI) m z: calc. for C₁₉H₂₃N₅O₂ 353.43; found, 354.2 [M+H]⁺; HPLC purity: 100%, t_{R}=16.326 min.

### Example 7. 4-[5-(5-Isopropoxy-1H-indazol-3-yl)-1,2,4-triazin-3-yl]morpholine (Compound 13)

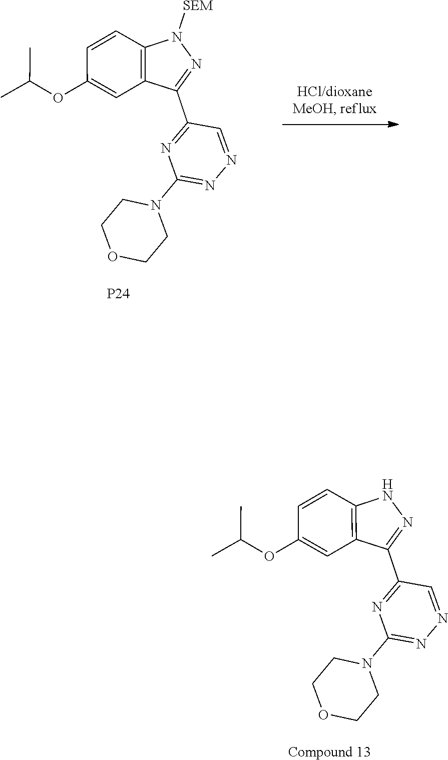 [SMILES: CC(C)Oc1ccc2c(c1)c(-c1cnnc(N3CCOCC3)n1)nn2COCC[Si](C)(C)C ; "P24" ; CC(C)Oc1ccc2[nH]nc(-c3cnnc(N4CCOCC4)n3)c2c1 ; "Compound 13"]

To a solution of P24 (30 mg, 0.063 mmol) in MeOH (5.0 mL) was added HCl (4N in dioxane, 5.0 mL) at rt. After the solution was stirred at 65° C. for overnight, the mixture was concentrated and purified by preparative TLC (n-hexane:EtOAc=1:1) to give Compound 13 (10 mg, 46% yield). ¹H NMR (400 MHz, CDCl₃), δ: 9.38 (s, 1H), 7.89 (d, J=2.2 Hz, 1H), 7.47 (d, J=9.0 Hz, 1H), 7.13 (dd, J=2.4, 9.0 Hz, 1H), 4.57 (septet, J=6.4 Hz, 1H), 4.06-4.03 (m, 4H), 3.90-3.87 (m, 4H), 1.41 (d, J=6.4 Hz, 6H); LCMS (ESI) m/z calc. for C₁₇H₂₀N₆O₂ 340.39; found, 341.2 [M+H]⁺; HPLC purity: 99.77%, t_{R}=18.187 min.

### Example 8. 6-(5-Isopropoxy-1H-indazol-3-yl)-2-methyl-4-morpholino-pyridazin-3-one (Compound 14)

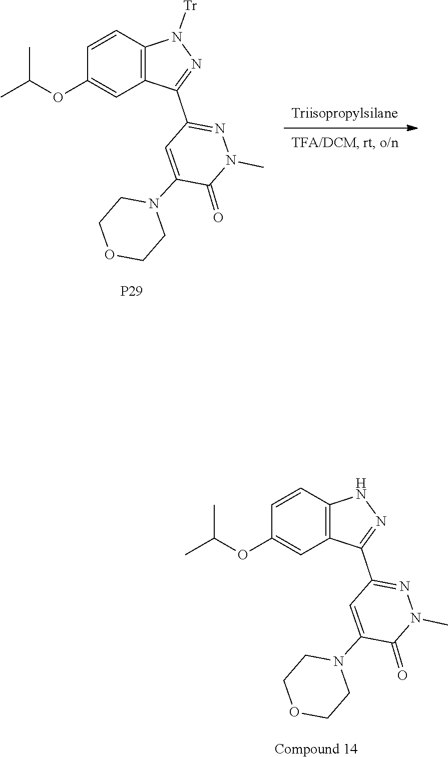

To a solution of P29 (2.30 g, 3.76 mmol) in DCM (62.5 mL) and TFA (12.5 mL) was added triisopropylsilane (0.772 mL, 3.76 mmol). After the mixture was stirred at rt for overnight, the mixture was concentrated under reduced pressure. The residue was purified by silica gel chromatography (0-60% EtOAc in n-hexane) to give a solid. The resulting solid was washed with methanol to give Compound 14 (310 mg, 22%) as a white solid. ¹H NMR (400 MHz, CDCl₃), δ: 10.01 (br. s, 1H), 7.87 (d, J=2.4 Hz, 1H), 7.37 (dd, J=9.0 Hz, 1H), 7.24 (s, 1H), 7.09 (dd, J=9.0, 2.4 Hz, 1H), 4.60 (septet, d, J=6.0 Hz, 1H), 3.90-3.86 (m, 7H), 3.54-3.52 (m, 4H), 1.40 (d, J=6.0 Hz, 6H); LCMS (ESI) m z: calc. for C₁₉H₂₃N₅O₃ 369.18; found, 370.2 [M+H]⁺; HPLC purity: 99.8%, t_{R}=18.519 min.

### Example 9. 6-(4-isopropoxy-1H-indazol-3-yl)-2-methyl-4-morpholino-pyridazin-3-one (Compound 15)

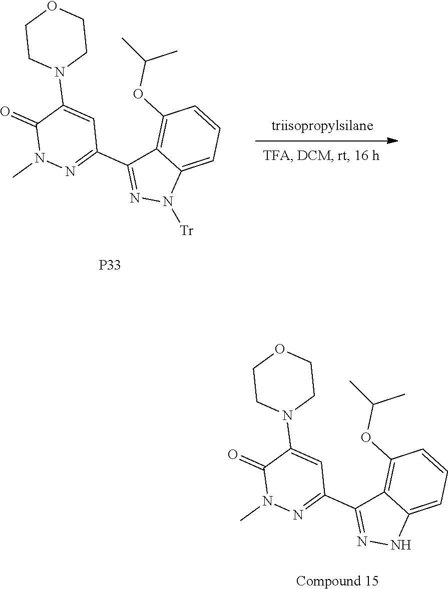

To a solution of P33 (400 mg, 0.654 mmol) in DCM (4.0 mL) was added TFA (4.0 mL) and triisopropylsilane (0.140 mL, 0.687 mmol). After the mixture was stirred at rt for overnight, the mixture was treated with saturated sodium bicarbonate (20 mL) and extracted with DCM. The combined organic layers were dried over MgSO₄, filtered, and concentrated under reduced pressure. The residue was purified by reverse phase chromatography (0-100% MeOH in H₂O) to give Compound 15 (12 mg, 5%) as a white solid. ¹H NMR (400 MHz, CDCl₃), δ: 10.19 (br. s, 1H), 7.32 (dd, J=8.4, 8.0 Hz, 1H), 7.06 (d, J=8.4 Hz, 1H), 6.91 (s, 1H), 6.56 (d, J=8.0 Hz, 1H), 4.70 (septet, J=6.0 Hz, 1H), 3.94-3.83 (m, 7H), 3.56-3.47 (m, 4H), 1.33 (d, J=6.4 Hz, 6H); LCMS (ESI) m/z: calc. for C₁₉H₂₃N₅O₃ 369.18; found, 370.5 [M+H]⁺; HPLC purity: 99.7%, t_{R}=15.836 min.

### Example 10. 2-[1-(6-pyrrolidin-1-ylpyridazin-4-yl)indazol-6-yl]thiolane 1,1-dioxide (Compound 34)

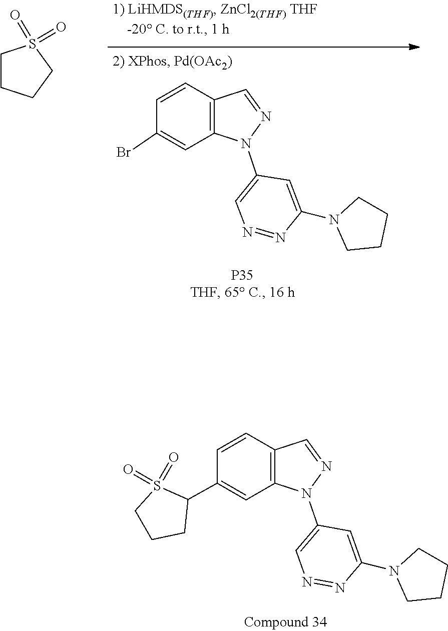 [SMILES: O=S1(=O)CCCC1 ; Brc1ccc2cnn(-c3cnnc(N4CCCC4)c3)c2c1 ; O=S1(=O)CCCC1c1ccc2cnn(-c3cnnc(N4CCCC4)c3)c2c1]

To a solution of tetrahydrothiophene 1,1-dioxide (30.0 mg, 0.25 mmol, 1.0 eq) in dry THF (0.5 mL) was added lithium bis(trimethylsilyl)amide (LiHMDS, 1.0 M in THF, 0.37 mL, 0.37 mmol, 1.5 eq) at −20° C. The reaction mixture was warmed to rt and stirred for 30 min. To the reaction mixture was added zinc chloride (0.5 M in THF, 0.75 mL, 0.37 mmol, 1.5 eq) at −20° C., and then the reaction mixture was stirred at rt for another 1 h. To the reaction mixture was added P35 (60.2 mg, 0.18 mmol, 0.7 eq) and a solution of dicyclohexyl[2′,4′,6′-tris(propan-2-yl)[1,1′-biphenyl]-2-yl]phosphane (XPhos, 13.1 mg, 0.027 mmol, 0.11 eq) and palladium(II) acetate (3.4 mg, 0.015 mmol, 0.06 eq) in dry THF (0.50 mL). The reaction mixture was degassed by argon for 10 min and then stirred at 65° C. for 16 h. The mixture was cooled to rt, quenched with saturated ammonium chloride aqueous solution, and extracted with DCM for three times. The combined organic layers were dried over Na₂SO₄₍ₛ₎, filtered and concentrated in vacuo to give the residue, which was purified by C-18 reversed-phase column chromatography (mobile phase A: water (with NH₄HCO₃), mobile phase B: methanol, UV: 214 and 254 nm, Flow rate: 35 mL/min, Gradient: 3-90% (% B)) to give Compound 34 (10 mg, 10% yield) as a white solid. ¹H NMR (400 MHz, CDCl₃), δ: 9.12 (d, J=2.0 Hz, 1H), 8.27 (d, J=1.0 Hz, 1H), 7.93 (s, 1H), 7.85 (d, J=8.6 Hz, 1H), 7.33 (dd, J=8.6, 1.0 Hz, 1H), 6.97 (d, J=2.0 Hz, 1H), 4.34 (dd, J=9.2, 6.8 Hz, 1H), 3.68-3.60 (m, 4H), 3.40-3.33 (m, 1H), 3.27-3.19 (m, 1H), 2.69-2.60 (m, 1H), 2.59-2.51 (m, 1H), 2.49-2.40 (m, 1H), 2.35-2.24 (m, 1H), 2.12-2.06 (m, 4H); LCMS (ESI) m/z: calc. for C₁₉H₂₁N₅O₂S 383.14; found, 384.2 [M+H]⁺; HPLC purity: 98.8%, t_{R}=12.300 min.

### Example 11. 2-methyl-2-[1-(6-pyrrolidin-1-ylpyridazin-4-yl)indazol-6-yl]propanenitrile (Compound 35)

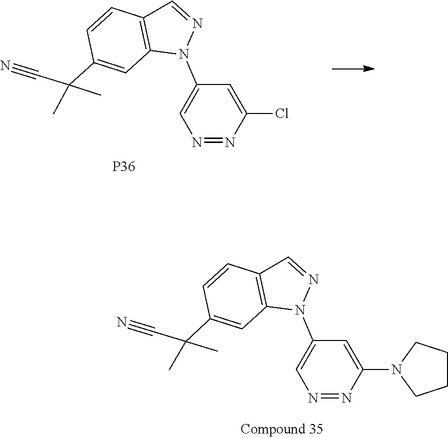

150 mg of compound P36 (0.505 mmol) and 3 mL of pyrrolidine was placed into a vial, sealed, and heated up to 120° C. for 12 h. The reaction mixture was concentrated under reduced pressure and purified via HPLC (YMC-Pack ODS-AQ 250×20 mml, S-10 μm, 12 nm, gradient water-acetonitrile). Yield of the Compound 35 is 52 mg (31%). LCMS (C18 column 20×2 mm, 2.5 μm, pore size 100 Å, water-acetonitrile+0.1% TFA, gradient 5 to 87% for 10 min, t_{R}=4.53 min). MS (ESI) m/z: 333.7 [MH]⁺. ¹H NMR (400 MHz, DMSO-d₆), δ: 9.07 (d, J=2.2 Hz, 1H), 8.54 (s, 1H), 8.10 (s, 1H), 8.01 (d, J=8.5 Hz, 1H), 7.55 (dd, J=8.5, 1.5 Hz, 1H), 7.14 (d, J=2.2 Hz, 1H), 3.56 (m, 4H), 2.02 (m, 4H), 1.80 (s, 6H).

### Example 12. 1-(1-(6-(pyrrolidin-1-yl)pyridazin-4-yl)-1H-indazol-6-yl)cyclohexanecarbonitrile (Compound 37)

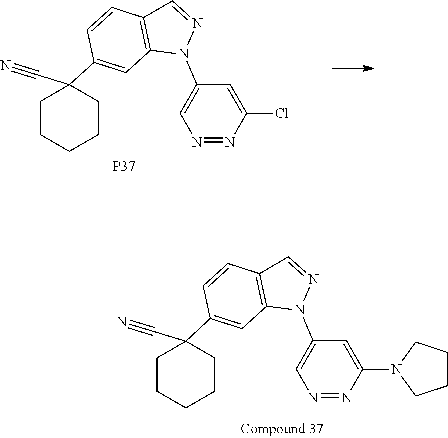

113 mg of compound P37 (0.335 mmol) and 3 mL of pyrrolidine was placed into a vial, sealed, and heated up to 120° C. for 12 h. The reaction mixture was concentrated under reduced pressure and purified via HPLC (YMC-Pack ODS-AQ 250×20 mml, S-10 μm, 12 nm, gradient water-acetonitrile). Yield of the Compound 37 87 mg (70%). LCMS (C18 column 20×2 mm, 2.5 μm, pore size 100 Å, water-acetonitrile+0.1% TFA, gradient 5 to 87% for 10 min, t_{R}=5.03 min). MS (ESI) m/z: 373.6 [MH]⁺. ¹H NMR (400 MHz, DMSO-d₆), δ: 9.08 (d, J=2.1 Hz, 1H), 8.54 (s, 1H), 8.12 (s, 1H), 8.00 (d, J=8.5 Hz, 1H), 7.56 (dd, J=8.5, 1.5 Hz, 1H), 7.13 (d, J=2.2 Hz, 1H), 3.57 (m, 4H), 2.17 (d, J=13.1 Hz, 2H), 2.02 (m, 6H), 1.88 (d, J=14.0 Hz, 2H), 1.69 (m, 3H), 1.37 (m, 1H).

### Example 13. 1-[1-(6-pyrrolidin-1-ylpyridazin-4-yl)indazol-6-yl]cyclopentanecarbonitrile (Compound 39)

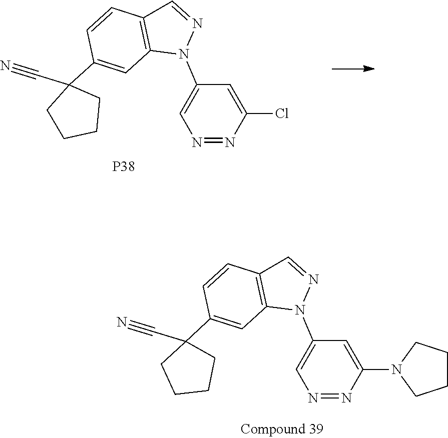

91 mg of compound P38 (0.282 mmol) and 3 mL of pyrrolidine was placed into a vial, sealed, and heated up to 120° C. for 12 h. The reaction mixture was concentrated under reduced pressure and purified via HPLC (YMC-Pack ODS-AQ 250×20 mml, 5-10 μm, 12 nm, gradient water-acetonitrile). Yield of Compound 39 was 54 mg (53%). LCMS (C18 column 20×2 mm, 2.5 μm, pore size 100 Å, water-acetonitrile+0.1% TFA, gradient 5 to 87% for 10 min, t_{R}=4.82 min). LCMS (ESI) m/z: 359.7 [M+H]⁺. ¹H NMR (400 MHz, DMSO-d₆), δ: 9.06 (d, J=2.1 Hz, 1H), 8.54 (s, 1H), 8.09 (s, 1H), 8.00 (d, J=8.5 Hz, 1H), 7.52 (dd, J=8.5, 1.5 Hz, 1H), 7.14 (d, J=2.2 Hz, 1H), 3.56 (m, 4H), 2.51 (m, 2H), 2.22 (m, 2H), 2.02 (m, 4H), 1.94 (m, 4H).

### Example 14. 1-[1-(6-pyrrolidin-1-ylpyridazin-4-yl)indazol-6-yl]pyrrole-2-carbonitrile (Compound 40)

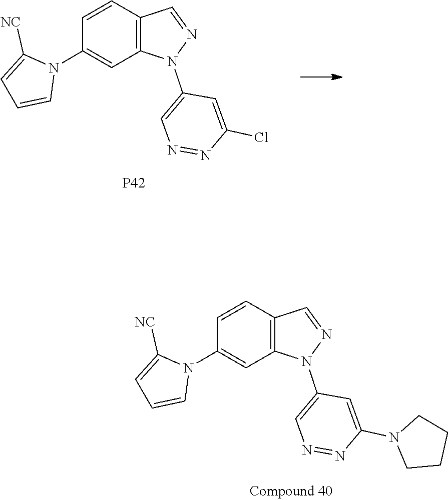

To a solution of compound P42 (0.17 g, 0.53 mmol) in THF (5 ml), pyrrolidine (0.1 ml, 0.085 g, 1.2 mmol). The resulted mixture was refluxed overnight and then evaporated in vacuo. The residue was partitioned between DCM and water (5 ml each). Organic layer was separated; aqueous was extracted with DCM (5 ml). Combined organic layers were dried over MgSO₄, filtered and concentrated. The residue was purified via silica gel chromatography (gradient elution: 100 to 60:1 CHCl₃/methanol) to provide 0.18 g (96%) of Compound 40. LCMS (C18 column 20×2 mm, 2.5 μm, pore size 100 Å, water-acetonitrile+0.1% TFA, gradient 5 to 87% for 10 min, retention time 4.74 min). LCMS (ESI) m/z: 356.2 [M+H]⁺. ¹H NMR (400 MHz, DMSO-d₆), δ: 9.53 (s, 1H), 9.32 (d, J=2.2 Hz, 1H), 8.03 (d, J=8.9 Hz, 1H), 7.95 (s, 1H), 7.65 (d, J=2.6 Hz, 1H), 7.44 (d, J=2.2 Hz, 1H), 7.33 (dd, J=8.9, 1.9 Hz, 1H), 7.27 (d, J=3.9 Hz, 1H), 6.49 (t, J=3.4 Hz, 1H), 3.57 (t, J=6.4 Hz, 4H), 2.02 (m, 4H).

### Example 15. 1-[1-(1-methyl-5-morpholino-6-oxo-pyridazin-3-yl)indazol-6-yl]cyclopropanecarbonitrile (Compound 41)

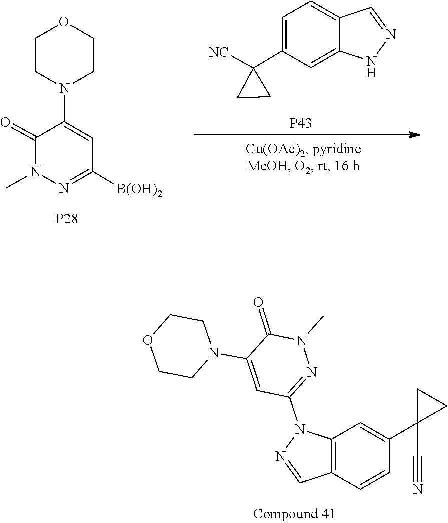 [SMILES: "P28" ; Cn1nc(B(O)O)cc(N2CCOCC2)c1=O ; Cn1nc(-n2ncc3ccc(C4(C#N)CC4)cc32)cc(N2CCOCC2)c1=O ; "P43" ; N#CC1(c2ccc3cn[nH]c3c2)CC1]

To a solution of (1-methyl-5-morpholino-6-oxo-1,6-dihydropyridazin-3-yl)boronic acid (P28, 2.79 mmol) in methanol (28 mL) was added 1-(1H-indazol-6-yl)cyclopropane-1-carbonitrile (P43, 510 mg, 2.79 mmol), copper(II) acetate (507 mg, 2.79 mmol) and pyridine (0.450 mL, 5.58 mmol). The mixture was bubbled with oxygen for 15 min. After the mixture was stirred at rt under oxygen atmosphere for overnight, the mixture was concentrated and treated with EtOAc. The solution was filtered through a pad of celite. The filtrate was concentrated in vacuo and then purified by silica gel chromatography (0-20% EtOAc in DCM) to give a solid. The resulting solid was washed with methanol to give Compound 41 (317 mg, 30%) as a white solid. ¹H NMR (400 MHz, CDCl₃), δ: 8.56 (s, 1H), 8.12 (s, 1H), 7.73 (d, J=8.4 Hz, 1H), 7.23 (s, 1H), 7.10 (d, J=8.4 Hz, 1H), 3.93-3.85 (m, 7H), 3.65-3.60 (m, 4H), 1.87-1.81 (m, 2H), 1.56-1.51 (m, 2H); LCMS (ESI) m/z: calc. for C₂₀H₂₀N₆O₂ 376.16; found, 377.0 [M+H]⁺; HPLC purity: 97.8%, t_{R}=19.279 min.

### Example 16. 2-methyl-2-[1-(1-methyl-5-morpholino-6-oxo-pyridazin-3-yl)indazol-6-yl]propanenitrile (Compound 43)

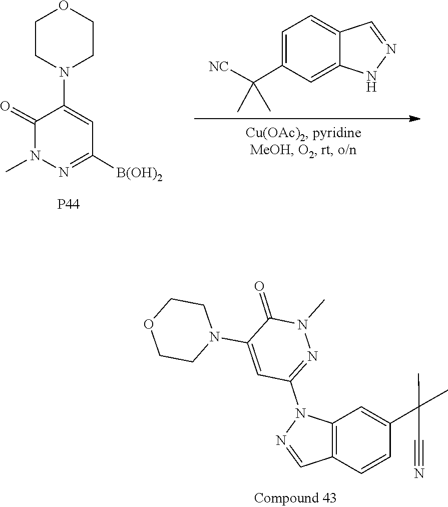 [SMILES: "P44" ; Cn1nc(B(O)O)cc(N2CCOCC2)c1=O ; Cn1nc(-n2ncc3ccc(C(C)(C)C#N)cc32)cc(N2CCOCC2)c1=O ; CC(C)(C#N)c1ccc2cn[nH]c2c1]

To a solution of P44 (1.31 mmol) in methanol (5 mL) was added 2-(1H-indazol-6-yl)-2-methylpropanenitrile (242 mg, 1.31 mmol), copper (II) acetate (237 mg, 1.31 mmol) and pyridine (0.21 mL, 2.6 mmol). The mixture was bubbled with oxygen for 15 min. After the mixture was stirred at rt under oxygen atmosphere for overnight, the mixture was concentrated and diluted with EtOAc. The solution was filtered through a pad of celite. The filtrate was concentrated in vacuo and then purified by silica gel chromatography (0-100% EtOAc in n-hexane). The resulting solid was washed with methanol to give Compound 43 (65 mg, 13%) as a white solid. ¹H NMR (400 MHz, CDCl₃), δ: 8.74 (s, 1H), 8.14 (d, J=1.2 Hz, 1H), 7.78 (d, J=8.4 Hz, 1H), 7.37 (dd, J=1.2 Hz, 8.4 Hz, 1H), 7.24 (s, 1H), 3.91-3.89 (m, 7H), 3.64-3.62 (m, 4H), 1.83 (s, 6H). LCMS (ESI) m/z: calc. for C₂₀H₂₂N₆O₂ 378.18; found, 379.0 [M+H]⁺; HPLC purity: 95.22%, t_{R}=20.149 min.

### Example 17. 2-Cyclopropyl-6-(5-isopropoxy-1H-indazol-3-yl)-4-morpholinopyridazin-3(2H)-one (Compound 19)

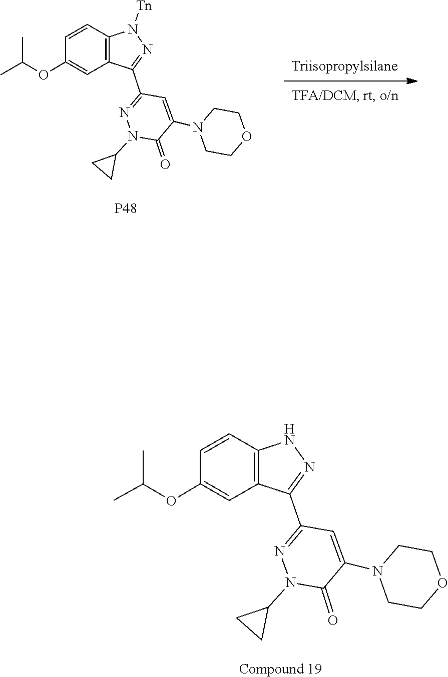

To a solution of P48 (250 mg, 0.392 mmol) in DCM (2.0 mL) was added trifluoroacetic acid (2.0 mL) and triisopropylsilane (0.085 mL, 0.41 mmol). After the mixture was stirred at rt for overnight, the mixture was treated with saturated sodium bicarbonate solution (10 mL) and extracted with DCM. The combined organic layers were dried over MgSO₄, filtered, and concentrated. The residue was purified by reverse phase chromatography (0-100% MeOH in H₂O) to give compound 19 (24 mg, 15%) as a white solid. ¹H NMR (400 MHz, CDCl₃), δ: 9.97 (s, 1H), 7.77 (d, J=2.4 Hz, 1H), 7.38 (d, J=8.8 Hz, 1H), 7.25 (s, 1H), 7.09 (dd, J=8.8, 2.4 Hz, 1H), 4.70-4.58 (m, 1H), 4.35-4.24 (m, 1H), 3.94-3.86 (m, 4H), 3.59-3.51 (m, 4H), 1.44 (d, J=6.0 Hz, 6H), 1.37-1.28 (m, 2H), 1.14-1.05 (m, 2H); LCMS (ESI) m/z calc. for C₂₁H₂₅N₅O₃ 395.20; found, 396.2 [M+H]⁺; HPLC purity: 97.46%, t_{R}=8.00 min.

### Example 18. 2-Methyl-6-(5-(1-methylcyclopropoxy)-1H-indazol-3-yl)-4-morpholinopyridazin-3(2H)-one (Compound 27)

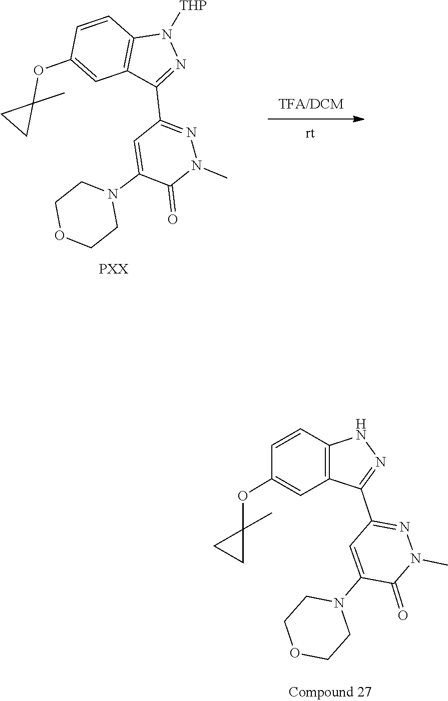

To a solution of P51 (943 mg, 1.45 mmol) in DCM (8.0 mL) was added trifluoroacetic acid (2 mL), then stirred at rt for overnight. The mixture was concentrated and purified by silica gel chromatography (0-100% EtOAc in n-hexane) to give compound 27 (190 mg, 36% yield) as a white solid. ¹H NMR (400 MHz, CDCl₃), δ: 8.08 (s, 1H), 7.39 (d, J=8.8 Hz, 1H), 7.25 (s, 1H), 7.13 (d, J=8.4 Hz, 1H), 3.91-3.88 (m, 7H), 3.54 (br. s, 4H), 1.63 (s, 3H), 1.17-1.01 (m, 2H), 0.77-0.74 (m, 2H); LCMS (ESI) m/z calc. for C₂₀H₂₃N₅O₃ 381.18; found, 382.5 [M+H]⁺; HPLC purity: 94%, t_{R}=18.990 min.

### Example 19. 6-(5-Cyclopropoxy-1H-indazol-3-yl)-2-methyl-4-morpholinopyridazin-3(2H)-one (Compound 30)

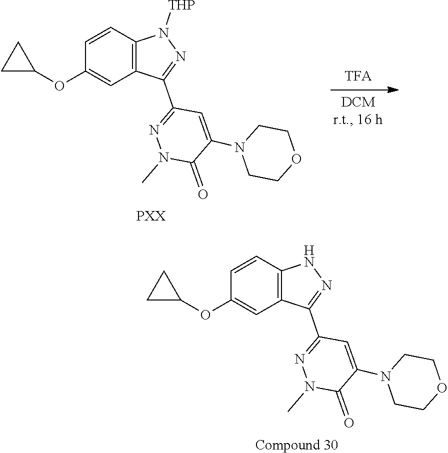

To a solution of P60 (41.0 mg, 0.0908 mmol, 1.0 eq) in DCM (1.5 mL) was added trifluoroacetic acid (0.50 mL). After the solution was stirred at rt for 16 h, the mixture solution was quenched with NaHCO_{3(aq)} and extracted with DCM. The organic layers were dried over MgSO₄₍ₛ₎, filtered, and concentrated under reduced pressure. The residue was purified by silica gel column chromatography (50% EtOAc in n-hexane) to afford compound 30 (13.9 mg, 42%) as a white solid. ¹H NMR (400 MHz, CDCl₃), δ: 13.2 (br. s, 1H), 7.99 (d, J=2.4 Hz, 1H), 7.48 (d, J=8.8 Hz, 1H), 7.21 (s, 1H), 7.07 (dd, J=9.2, 2.4 Hz, 1H), 3.90-3.85 (m, 1H), 3.77-3.73 (m, 7H), 3.47-3.45 (m, 4H), 0.84-0.79 (m, 2H), 0.71-0.67 (m, 2H); LCMS (ESI) m z calc. for C₁₉H₂₁N₅O₃ 367.1; found, 368.3 [M+H]⁺; HPLC purity: 99.2%, t_{R}=17.955 min.

### Example 20. 4-((3aR,6aS)-3,3-dimethylhexahydro-1H-furo[3,4-b]pyrrol-1-yl)-2-methyl-6-(5-(1-methylcyclopropoxy)-1-(tetrahydro-2H-pyran-2-yl)-1H-indazol-3-yl)pyridazin-3(2H)-one (Compound 48)

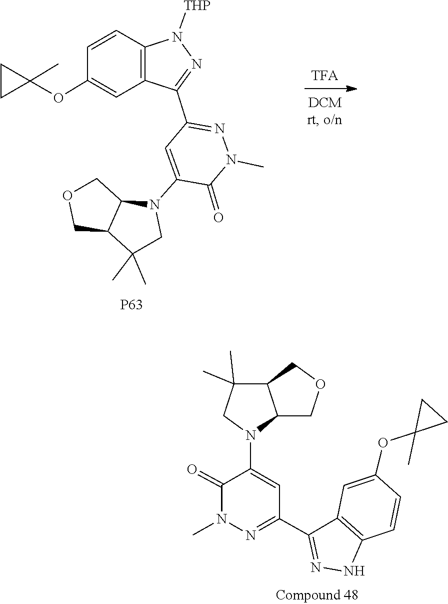

To a solution of P63 (69 mg, 0.13 mmol) in dichloromethane (4.0 mL) was added 2,2,2-trifluoroacetic acid (1.0 mL) and then stirred at rt for overnight. The mixture was concentrated, diluted with DCM, and adjusted pH value to 7 with Dowex-OH. The mixture was concentrated in vacuo and then purified by C18 reverse phase column (0-100% MeOH in water) to give compound 48 (11 mg, 19%) as a yellow solid. ¹H NMR (400 MHz, CDCl₃), δ: 8.09 (d, J=2.4 Hz, 1H), 7.36 (d, J=9.0 Hz, 1H), 7.10 (dd, J=9.0, 2.4 Hz, 1H), 6.80 (s, 1H), 5.39 (br. s, 1H), 4.04-4.00 (m, 1H), 3.87 (s, 3H), 3.82-3.71 (m, 3H), 3.38 (br. s, 2H), 2.55-2.52 (m, 1H), 1.63 (s, 3H), 1.16 (s, 3H), 1.14 (s, 3H), 1.09-1.08 (m, 2H), 0.77-0.74 (m, 2H); LCMS (ESI) m/z calc. for C₂₄H₂₉N₅O₃ 435.23; found, 436.3 [M+H]⁺; HPLC purity: 95.4%, t_{R}=22.069 min.

### Example 21. 4-((2S,6R)-2,6-dimethylmorpholino)-2-methyl-6-(5-(1-methylcyclopropoxy)-1H-indazol-3-yl)pyridazin-3(2H)-one (Compound 50)

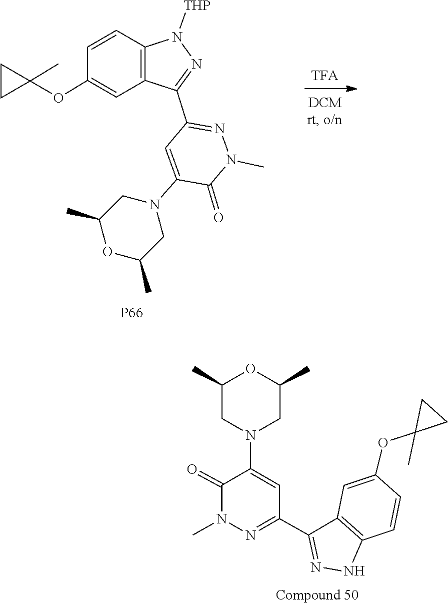

To a solution of P66 (423 mg, 0.65 mmol) in dichloromethane (4.0 mL) was added 2,2,2-trifluoroacetic acid (1.0 mL) and then stirred at rt for overnight. The mixture was concentrated, diluted with DCM, and adjusted pH value to 7 with Dowex-OH. The mixture was concentrated in vacuo and then purified by C18 reverse phase column (0-100% MeOH in water) to give compound 50 (50 mg, 14%) as an orange solid. ¹H NMR (400 MHz, CDCl₃), δ: 8.08 (d, J=2.2 Hz, 1H), 7.39 (d, J=8.8 Hz, 1H), 7.21 (s, 1H), 7.13 (dd, J=8.8, 2.2 Hz, 1H), 4.15-4.12 (m, 2H), 3.91-3.88 (m, 5H), 2.53-2.47 (m, 2H), 1.26-1.24 (m, 9H), 1.11-1.08 (m, 2H), 0.78-0.75 (m, 2H); LCMS (ESI) m/z calc. for C₂₂H₂₇N₅O₃ 409.21; found, 410.3 [M+H]⁺; HPLC purity: 97.6%, t_{R}=21.583 min.

### Example 22. 2-Cyclopropyl-4-((2S,6R)-2,6-dimethylmorpholino)-6-(5-(1-methylcyclopropoxy)-1H-indazol-3-yl)pyridazin-3(2H)-one (Compound 51)

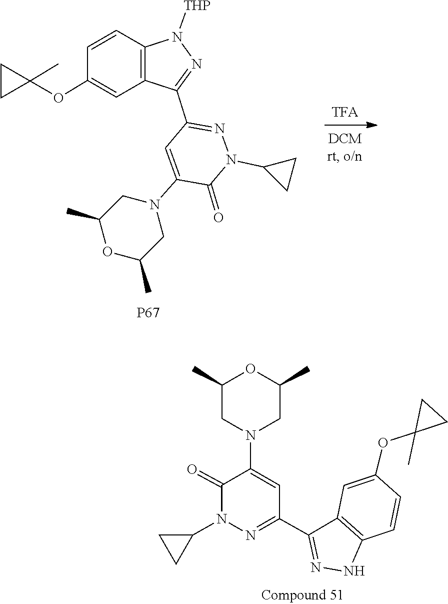

To a solution of P67 (130 mg, 0.25 mmol) in dichloromethane (4.0 mL) was added 2,2,2-trifluoroacetic acid (1.0 mL) and then stirred at rt for overnight. The mixture was concentrated, diluted with DCM, and adjusted pH value to 7 with Dowex-OH. The mixture was concentrated in vacuo and then purified by C18 reverse phase column (0-100% MeOH in water) to give compound 51 (6.5 mg, 6%) as an orange solid. ¹H NMR (400 MHz, CDCl₃), δ: 7.98 (d, J=2.2 Hz, 1H), 7.38 (d, J=9.0 Hz, 1H), 7.24 (s, 1H), 7.05 (dd, J=9.0, 2.2 Hz, 1H), 4.38-4.32 (m, 1H), 4.15-4.12 (m, 2H), 3.93-3.88 (m, 2H), 2.53-2.47 (m, 2H), 1.67 (s, 3H), 1.38-1.34 (m, 2H), 1.26 (s, 3H), 1.25 (s, 3H), 1.10-1.05 (m, 4H), 0.84-0.80 (m, 2H); LCMS (ESI) m/z calc. for C₂₄H₂₉N₅O₃ 435.23; found, 436.3 [M+H]⁺; HPLC purity: 96.9%, t_{R}=23.664 min.

### Example 22. 2-Methyl-6-(5-(1-methylcyclopropoxy)-1H-indazol-3-yl)-4-(5-oxa-8-azaspiro[2.6]nonan-8-yl)pyridazin-3(2H)-one (Compound 52)

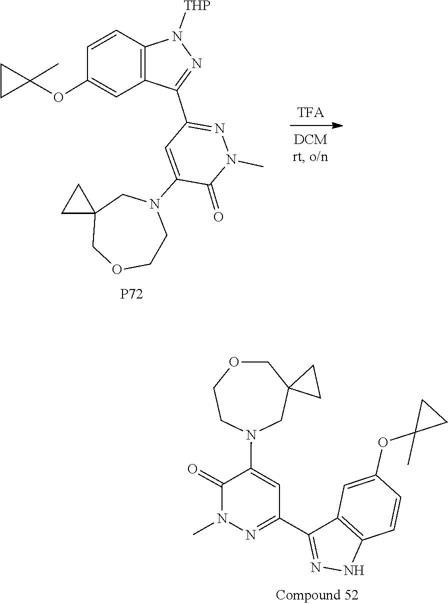

To a solution of P72 (160 mg, 0.32 mmol) in dichloromethane (4.0 mL) was added 2,2,2-trifluoroacetic acid (1.0 mL) and then stirred at rt for overnight. The mixture was concentrated, diluted with DCM, and adjusted pH value to 7 with Dowex-OH. The mixture was concentrated in vacuo and then purified by C18 reverse phase column (0-100% MeOH in water) to give compound 52 (51.1 mg, 38%) as a yellow solid. ¹H NMR (400 MHz, CD₃OD), δ: 8.06 (d, J=2.2 Hz, 1H), 7.42 (d, J=8.8 Hz, 1H), 7.18 (s, 1H), 7.06 (dd, J=8.8, 2.2 Hz, 1H), 4.13-4.07 (m, 2H), 3.99-3.96 (m, 2H), 3.86 (s, 3H), 3.62 (s, 2H), 3.55 (s, 2H), 1.61 (s, 3H), 1.04-1.00 (m, 2H), 0.81-0.77 (m, 2H), 0.74-0.67 (m, 4H); LCMS (ESI) m/z calc. for C₂₃H₂₇N₅O₃ 421.21; found, 422.3 [M+H]⁺; HPLC purity: 97.9%, t_{R}=21.332 min.

### Example 23. 4-(3,3-Difluoropiperidin-1-yl)-2-methyl-6-(5-(1-methylcyclopropoxy)-1H-indazol-3-yl)pyridazin-3(2H)-one (Compound 54)

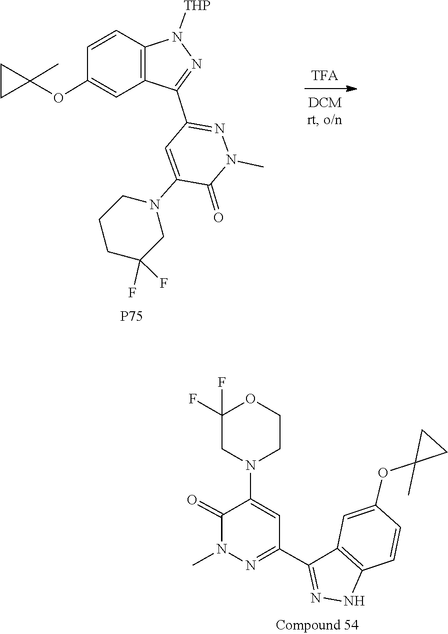

To a solution of P75 (93 mg, 0.19 mmol) in DCM (4.0 mL) was added 2,2,2-trifluoroacetic acid (1.0 mL) and then stirred at rt for overnight. The mixture was concentrated, diluted with DCM, and adjusted pH value to 7 with Dowex-OH. The mixture was concentrated in vacuo and then purified by C18 reverse phase column (0-100% MeOH in water) to give compound 54 (22.5 mg, 29%) as a yellow solid. ¹H NMR (400 MHz, CD₃OD), δ: 8.05 (d, J=2.0 Hz, 1H), 7.44-7.42 (m, 2H), 7.06 (dd, J=9.0, 2.2 Hz, 1H), 4.01 (t, ³J_{H-F}=11.6 Hz, 2H), 3.89 (s, 3H), 3.59-3.53 (m, 2H), 2.15-2.06 (m, 2H), 2.01-1.91 (m, 2H), 1.61 (s, 3H), 1.03-1.00 (m, 2H), 0.81-0.78 (m, 2H); LCMS (ESI) m/z calc. for C₂₁H₂₃F₂N₅O₂ 415.18; found, 416.3 [M+H]⁺; HPLC purity: 94.3%, t_{R}=22.263 min.

### Example 24. 2-Methyl-6-(5-(1-methylcyclopropoxy)-1H-indazol-3-yl)-4-(6-azaspiro[3.4]octan-6-yl)pyridazin-3(2H)-one (Compound 56)

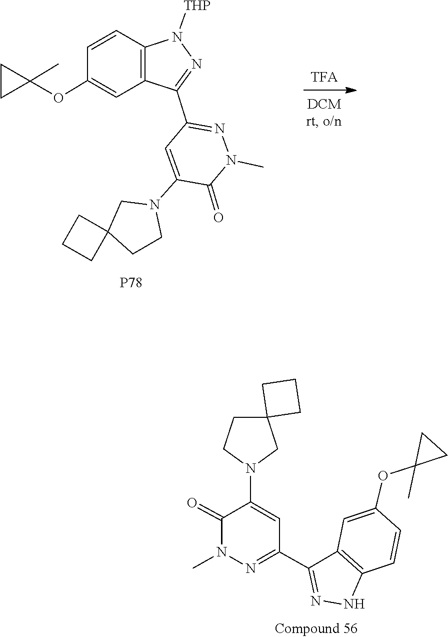

To a solution of P78 (72.3 mg, 0.15 mmol) in DCM (4.0 mL) was added 2,2,2-trifluoroacetic acid (1.0 mL), and the solution was stirred at rt for overnight. The mixture was concentrated, diluted with DCM, and adjusted pH value to 7 with Dowex-OH. The mixture was concentrated in vacuo and then purified by C18 reverse phase column (0-100% MeOH in water) to give compound 56 (22.4 mg, 37%) as a yellow solid. ¹H NMR (400 MHz, CD₃OD), δ: 8.06 (s, 1H), 7.42-7.39 (m, 1H), 7.04 (dd, J=9.0, 2.4 Hz, 1H), 6.82 (s, 1H), 3.82 (s, 3H), 3.75-3.70 (m, 4H), 2.1-1.94 (m, 8H), 1.61 (s, 3H), 1.03-1.00 (m, 2H), 0.80-0.77 (m, 2H).; LCMS (ESI) m/z calc. for C₂₃H₂₇N₅O₂ 405.22; found, 406.3 [M+H]⁺; HPLC purity: 91.8%, t_{R}=25.199 min.

### Example 25. 2-Cyclopentyl-6-(5-isopropoxy-1H-indazol-3-yl)-4-morpholinopyridazin-3(2H)-one (Compound 16)

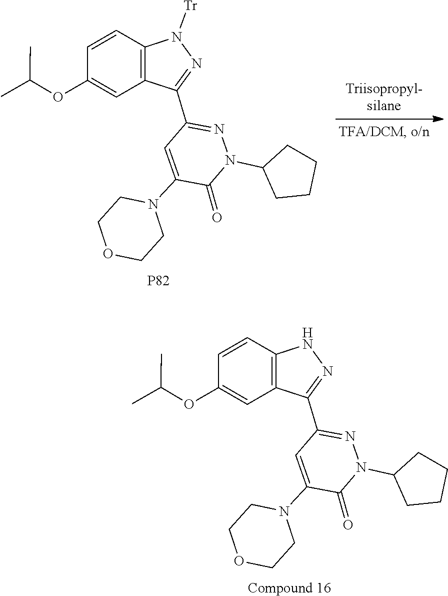

To a solution of P82 (1.2 g, 1.33 mmol) in DCM (22 mL) was added trifluoroacetic acid (5.0 mL) and triisopropylsilane (211 mg, 1.33 mmol). The reaction was stirred at rt for overnight. The mixture was concentrated and purified by silica gel chromatography (0-100% EtOAc in n-hexane) to give compound 16 (180 mg, 31% yield) as a white solid. ¹H NMR (400 MHz, CDCl₃), δ: 7.84 (d, J=2.4 Hz, 1H), 7.39 (d, J=9.2 Hz, 1H), 7.26 (s, 1H), 7.09 (dd, J=9.2, 2.4 Hz, 1H), 5.62-5.54 (m, 1H), 4.60 (septet, J=6.0 Hz, 1H), 3.91-3.88 (m, 4H), 3.53-3.50 (m, 4H), 2.20-2.09 (m, 4H), 1.99-1.91 (m, 2H), 1.79-1.74 (m, 2H), 1.40 (d, J=6.0 Hz, 6H); LCMS (ESI) m z calc. for C₂₃H₂₉N₅O₃ 423.23; found, 424.6 [M+H]⁺; HPLC purity: 99%, t_{R}=24.056 min.

### Example 26. 2-Methyl-4-morpholino-6-(5-(trifluoromethoxy)-1H-indazol-3-yl)pyridazin-3(2H)-one (Compound 22)

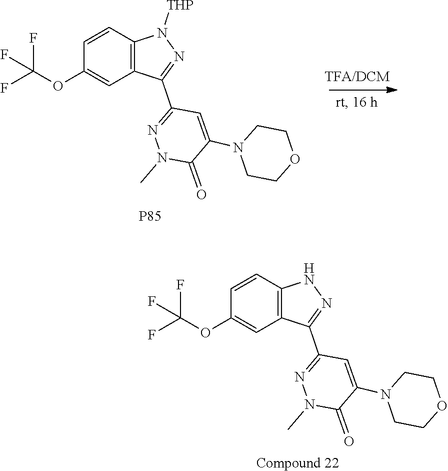

To a solution of P85 (400 mg, 0.834 mmol) in DCM (4.0 mL) was added trifluoroacetic acid (1.0 mL). After the mixture was stirred at rt for overnight, the mixture was treated with saturated sodium bicarbonate (10 mL) and extracted with DCM. The combined organic layers were dried over MgSO₄, filtered, and concentrated. The residue was purified by silica gel chromatography (0-5% methanol in dichloromethane) to give compound 22 (22.7 mg, 7%) as a white solid. ¹H NMR (400 MHz, DMSO-d₆), δ: 13.65 (br. s, 1H), 8.20 (s, 1H), 7.72 (d, J=9.2 Hz, 1H), 7.42 (dd, J=9.2, 2.4 Hz, 1H), 7.20 (s, 1H), 3.81-3.70 (m, 7H), 3.54-3.43 (m, 4H); LCMS (ESI) m/z calc. for C₁₇H₁₆F₃N₅O₃ 395.12; found, 396.3 [M+H]⁺; HPLC purity: 98.9%, t_{R}=20.199 min.

### Example 27. 6-(5-Isopropoxy-4-methyl-1H-indazol-3-yl)-2-methyl-4-morpholinopyridazin-3(2H)-one (Compound 24)

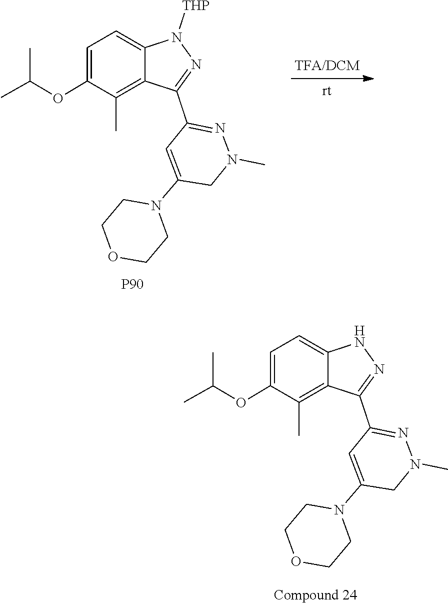

To a solution of P90 (323 mg, 0.69 mmol) in DCM (8.0 mL) was added trifluoroacetic acid (2.0 mL). The solution was stirred at rt for overnight. The mixture was concentrated and purified by silica gel chromatography (0-100% EtOAc in n-hexane) to give compound 24 (190 mg, 71% yield) as a white solid. ¹H NMR (400 MHz, CDCl₃), δ: 10.07 (s, 1H), 7.27 (d, J=8.8 Hz, 1H), 7.17 (d, J=8.8 Hz, 1H), 6.84 (s, 1H), 4.38 (septet, J=6.0 Hz, 1H), 3.89-3.86 (m, 7H), 3.54-3.51 (m, 4H), 2.47 (s, 3H), 1.33 (d, J=6.0 Hz, 6H); LCMS (ESI) m z calc. for C₂₀H₂₅N₅O₃ 383.20; found, 384.5 [M+H]⁺; HPLC purity: 99%, t_{R}=18.114 min.

### Example 28. 2-Methyl-4-morpholino-6-(3,7,8,9-tetrahydropyrano[3,2-e]indazol-1-yl)pyridazin-3(2H)-one (Compound 29)

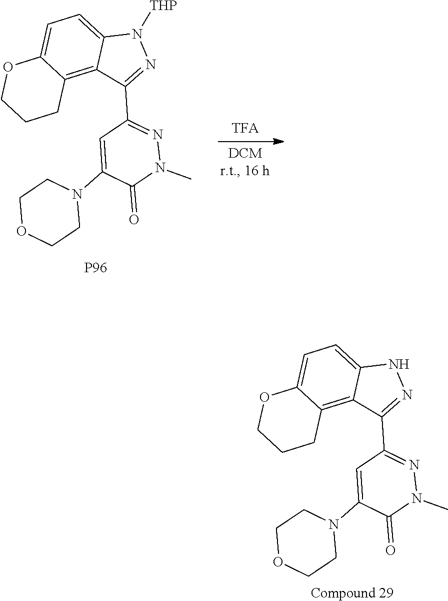

To a solution of P96 (35.0 mg, 0.0775 mmol, 1.0 eq) in DCM (1.5 mL) was added trifluoroacetic acid (0.50 mL) and it was stirred at rt for 16 h. The mixture was quenched with NaHCO_{3(aq)} and extracted with DCM. The organic layers were dried over MgSO₄₍ₛ₎, filtered, and concentrated under reduced pressure. The residue was purified by silica gel column chromatography (50% EtOAc in n-hexane) to afford compound 29 (9.3 mg, 33%) as a white solid. ¹H NMR (400 MHz, CDCl₃), δ: 9.81 (br. s, 1H), 7.23 (d, J=8.8 Hz, 1H), 6.98 (d, J=8.8 Hz, 1H), 6.88 (s, 1H), 4.23-4.21 (m, 2H), 3.89-3.86 (m, 7H), 3.54-3.52 (m, 4H), 3.02 (t, J=6.8 Hz, 2H), 2.04-1.98 (m, 2H); LCMS (ESI) m z calc. for C₁₉H₂₁N₅O₃ 367.1; found, 368.3 [M+H]⁺; HPLC purity: 98.36%, t_{R}=15.303 min.

### Example 29. 2-Methyl-4-morpholino-6-(1,6,7,8-tetrahydropyrano[2,3-f]indazol-3-yl)pyridazin-3(2H)-one (Compound 31)

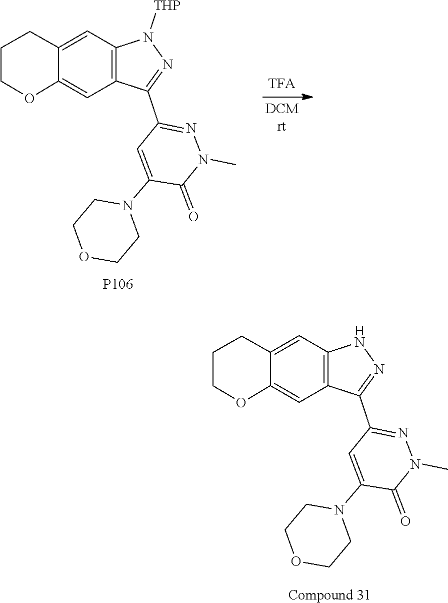

To a solution of P106 (128 mg, 0.28 mmol) in DCM (4.0 mL) was added trifluoroacetic acid (1.0 mL). The solution was stirred at rt for overnight. The mixture was concentrated and purified by silica gel chromatography (0-100% EtOAc in n-hexane) to give compound 31 (45.9 mg, 44% yield) as a yellow solid. ¹H NMR (400 MHz, CDCl₃), δ: 7.78 (s, 1H), 7.23 (s, 1H), 7.16 (s, 1H), 4.28-4.24 (m, 2H), 3.89-3.87 (m, 7H), 3.55-3.52 (m, 4H), 3.01 (t, J=6.6 Hz, 2H), 2.10-2.04 (m, 2H); LCMS (ESI) m/z calc. for C₁₉H₂₁N₅O₃ 367.16; found, 368.2 [M+H]⁺; HPLC purity: 98%, t_{R}=16.564 min.

### Example 30. 6-(5-(Difluoromethoxy)-1H-indazol-3-yl)-2-methyl-4-morpholinopyridazin-3(2H)-one (Compound 32)

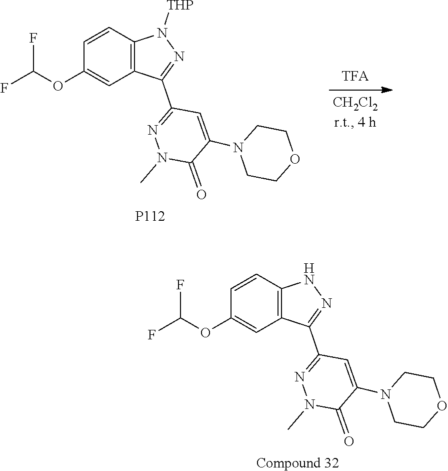

To a solution of P112 (104 mg, 0.225 mmol, 1.0 eq) in DCM (1.2 mL) was added trifluoroacetic acid (1.0 mL) slowly. After the solution was stirred at rt for 4 h, the solution was treated with sat. NaHCO_{3(aq)}, adjusted pH to 7 and extracted with DCM three times. The combined organic layers were dried over Na₂SO₄₍ₛ₎, filtered, and concentrated in vacuo to give a residue, which was purified by C-18 reversed-phase column chromatography (mobile phase A: water (with 0.1% NH₄HCO₃), mobile phase B: acetonitrile, UV: 214 and 254 nm, Flow rate: 40 mL/min, Gradient: 3-90% (% B)) to give compound 32 (41 mg, 48% yield) as a white solid. ¹H NMR (400 MHz, CDCl₃), δ: 8.20-8.18 (m, 1H), 7.49 (dd, J=8.8, 0.8 Hz, 1H), 7.28 (dd, J=9.2, 2.4 Hz, 1H), 7.24 (s, 1H), 6.57 (t, J=74.4 Hz, 1H), 3.92 (s, 3H), 3.91-3.87 (m, 4H), 3.58-3.54 (m, 4H); LCMS (ESI) m/z calc. for C₁₇H₁₇F₂N₅O₃ 377.13; found, 378.0 [M+H]⁺.

### Example 31. 2-Methyl-6-(5-(methylsulfonyl)-1H-indazol-3-yl)-4-morpholinopyridazin-3(2H)-one (Compound 33)

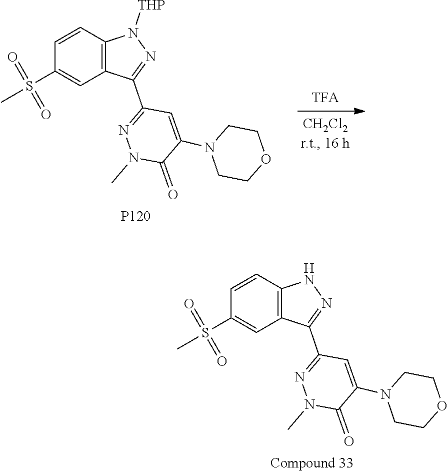

To a solution of P120 (80.0 mg, 0.169 mmol, 1.0 eq) in DCM (1.7 mL) was added trifluoroacetic acid (1.2 mL) slowly. After the solution was stirred at rt for 16 h, the solution was treated with saturated NaHCO_{3(aq)}, adjusted pH to 7 and extracted with DCM three times. The combined organic layers were dried over Na₂SO₄₍ₛ₎, filtered, and concentrated in vacuo to give a residue. The residue was purified by C-18 reversed-phase column chromatography (mobile phase A: water (with 0.1% NH₄HCO₃), mobile phase B: acetonitrile, UV: 214 and 254 nm, Flow rate: 40 mL/min, Gradient: 3-90% (% B)) to give compound 33 (56 mg, 85% yield) as a white solid. ¹H NMR (400 MHz, DMSO-d₆), δ: 13.88 (br. s, 1H), 8.87 (s, 1H), 7.93 (dd, J=8.8 Hz, 1H), 7.83 (d, J=8.8 Hz, 1H), 7.23 (s, 1H), 3.80 (s, 3H), 3.78-3.73 (m, 4H), 3.54-3.48 (m, 4H), 3.26 (s, 3H); LCMS (ESI) m/z calc. for C₁₇H₁₉N₅O₄S 389.12; found, 390.1 [M+H]⁺.

### Example 32. 4-(4-Fluoro-3,3-dimethylpyrrolidin-1-yl)-2-methyl-6-(5-(1-methylcyclopropoxy)-1-(tetrahydro-2H-pyran-2-yl)-1H-indazol-3-yl) pyridazin-3(2H)-one (Compound 55)

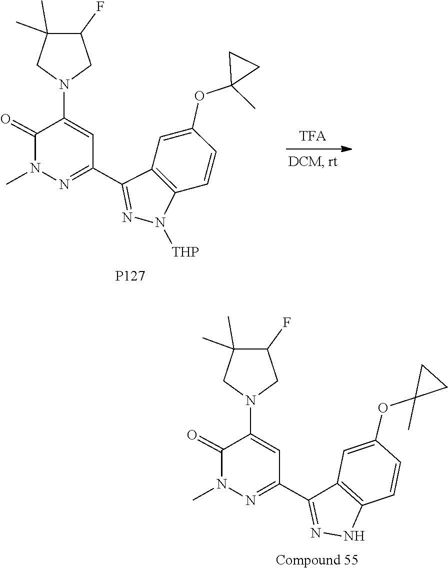

To a solution of 4-(4-fluoro-3,3-dimethylpyrrolidin-1-yl)-2-methyl-6-(5-(1-methylcyclopropoxy)-1-(tetrahydro-2H-pyran-2-yl)-1H-indazol-3-yl)pyridazin-3(2H)-one (P127, 75 mg, 0.23 mmol) in DCM (2 mL) was added trifluoroacetate (1 mL) at rt. After stirred at rt for 16 h, the mixture was diluted with water (10 mL) and DCM (20 mL) for three times. The combined organic layers were dried, filtered, and concentrated to leave a residue which was purified by C18 column (acetonitrile: 0.02% ammonium bicarbonate aqueous solution=05% to 95%) to afford the desired product (33.5 mg, 98.5% purity, 52% yield) as white solid. LCMS (ESI): R_{T}=3.868 min, mass calc. for C₂₂H₂₆FN₅O₂ 411.2, m/z found 412.1 [M+H]⁺. ¹H NMR (400 MHz, DMSO-d₆), δ: 13.13 (s, 1H), 7.97 (d, J=2.4 Hz, 1H), 7.46 (d, J=8.8 Hz, 1H), 7.03 (dd, J=9.2, 2.4 Hz, 1H), 6.74 (s, 1H), 4.82 (dd, J=53.6, 2.8 Hz, 1H), 4.28-4.23 (m, 1H), 4.16-4.12 (m, 1H), 3.90 (s, 3H), 3.72-3.43 (m, 2H), 1.55 (s, 3H), 1.17 (s, 3H), 1.02 (s, 3H), 0.97-0.94 (m, 2H), 0.80-0.77 (m, 2H).

### Biological Assays

### Example A. Primary Assays Used to Determine Potency of the LRRK2 Inhibition

### Assay for Determining Enzymatic Activity Inhibition Against LRRK2[G2019S].

Compound activity was determined using recombinant LRRK2[G2019S] protein (SignalChem Biotech, Inc., Cat #L10-12GG-10) and LRRKtide substrate (SignalChem Biotech, Inc., Cat #L10-58) in an in vitro enzymatic reaction. The enzymatic reaction was carried out in assay buffer (40 mM TRIS-HCl pH 7.4, 20 mM MgCl₂, 0.05 mM DTT, 0.1 mg/mL BSA). The compounds were dispensed into a 384 well Diamond Well Plate (Axigen, Cat #P-384-120SQ-C-S) using the Biomek FX liquid handling system at the 80× solutions of compounds in DMSO. The 2× Protein-Substrate mix (final concentration 36 nM of LRRK2[G2019S] and 0.8 μg/μL of LRRKtide) was prepared in 1× Assay buffer and 4 μL of mixture per well was added into the 384-well white Reaction plate with NBS (Corning, Cat #CLS4513). The 4 μL of LRRKtide substrate w/o LRRK2[G2019S] in the 1× buffer was used for a negative control. Plates were centrifuged for 1 min at 200 g. Next the compounds were added to the reaction plate using Biomek station via following steps: 1 μL of 80× compounds (in DMSO) were mixed thoroughly with 39 μL of 2×10 μM ATP in Assay Buffer, then 4 μL of this mixture was added to Reaction plate with 4 μL of Protein-Substrate mix. Plates were centrifuged for 1 min at 200 g and incubated for 1 h at rt. Then, 4 μL per well of ADP-Glo reagent (Promega, ADP-Glo™ Kinase Assay, Cat #V9102) was added. Plates were incubated for 30 min at rt. Then, 8 μL per well of Kinase detection reagent (Promega, ADP-Glo™ Kinase Assay, Cat #V9102) was added and the luminescence signal was measured with a microplate reader. The luminescence signal was detected with CLARIOstar Plus (BMG LABTECH) microplate reader. The % inhibition was then used to calculate the Ki values. The average Ki values are shown in Table A, wherein “A” corresponds to Ki<5.0 nM, “B” corresponds to 5.0 nM≤Ki<10.0 nM, “C” 10.0 nM≤Ki<50.0 nM, “D” corresponds to 50.0 nM≤Ki<100.0 nM and “E” corresponds to Ki>100.0 nM.

TABLE A
LRRK2 (G2019S) Activity Inhibition Assays

| Compound Number | Kᵢ, nM* |
|---|---|
| 1 | B |
| 2 | B |
| 3 | A |
| 4 | A |
| 5 | A |
| 10 | C |
| 13 | B |
| 14 | A |
| 15 | E |
| 16 | B |
| 18 | B |
| 19 | A |
| 21 | B |
| 22 | E |
| 23 | A |
| 24 | E |
| 26 | C |
| 27 | A |
| 28 | A |
| 29 | E |
| 30 | A |
| 31 | E |
| 32 | D |
| 33 | E |
| 34 | E |
| 35 | D |
| 36 | E |
| 37 | E |
| 39 | D |
| 41 | D |
| 43 | E |
| 44 | A |
| 45 | C |
| 48 | A |
| 50 | A |
| 51 | A |
| 52 | A |
| 54 | A |
| 56 | A |

*Kᵢ is the dissociation equilibrium constant of the enzyme-inhibitor complex (E-I). Ki values are determined through a series of experiments with varying amounts of inhibitor present. Ki: A ≤ 5 nM; 5 nM < B ≤ 10 nM; 10 nM < C ≤ 50 nM; 50 nM < D ≤ 100 nM; E > 100 nM

### Example B. Primary Assays Used to Determine Potency of the LRRK2 Inhibition

### Assay for Determining Enzymatic Activity Inhibition Against LRRK2 WT.

Compound activity was determined using recombinant LRRK2 WT protein (SignalChem Biotech, Inc., Cat #L10-11G-10) and LRRKtide substrate (SignalChem Biotech, Inc., Cat #L10-58) in an in vitro enzymatic reaction. The enzymatic reaction was carried out in assay buffer (40 mM TRIS-HCl pH 7.4, 20 mM MgCl₂, 0.05 mM DTT, 0.1 mg/mL BSA). The compounds were dispensed into a 384 well Diamond Well Plate (Axigen, Cat #P-384-120SQ-C-S) using the Biomek FX liquid handling system at 80× solutions of compounds in DMSO. The 2× Protein-Substrate mix (final concentration 36 nM of LRRK2 WT and 0.8 μg/μL of LRRKtide) was prepared in the 1× Assay buffer and 4 μL of mixture per well was added into the 384-well white Reaction plate with NBS (Corning, Cat #CLS4513). The 4 μL of LRRKtide substrate w/o LRRK2 WT in the 1× buffer was used for a negative control. Plates were centrifuged for 1 min at 200 g. Next the compounds were added to Reaction plate using Biomek station via following steps: 1 μL of 80× compounds (in DMSO) were mixed thoroughly with 39 μL of 2×10 μM ATP in Assay Buffer, then 4 μL of this mixture was added to Reaction plate with 4 μL of Protein-Substrate mix. Plates were centrifuged for 1 min at 200 g and incubated for 1 hour at rt. Then, 4 μL per well of ADP-Glo reagent (Promega, ADP-Glo™ Kinase Assay, Cat #V9102) was added. Plates were incubated for 30 minutes at rt. Then, 8 μL per well of Kinase detection reagent (Promega, ADP-Glo™ Kinase Assay, Cat #V9102) was added and the luminescence signal was measured with a microplate reader. The % inhibition was then used to calculate the Ki values. The average Ki values are shown in Table B, wherein “A” corresponds to Ki<10.0 nM, “B” corresponds to 10.0 nM K Ki<20.0 nM, “C” 20.0 nM K Ki<50.0 nM, “D” corresponds to 50.0 nM K Ki<100.0 nM and “E” corresponds to Ki>100.0 nM.

TABLE B
LRRK2 (WT) Activity Inhibition Assays

| Compound Number | Kᵢ, nM* |
|---|---|
| 1 | B |
| 2 | B |
| 3 | A |
| 4 | A |
| 5 | B |
| 10 | A |
| 13 | A |
| 14 | A |
| 15 | E |
| 16 | A |
| 18 | A |
| 19 | A |
| 21 | A |
| 22 | E |
| 23 | A |
| 24 | E |
| 26 | A |
| 27 | A |
| 28 | A |
| 29 | D |
| 30 | A |
| 31 | D |
| 32 | E |
| 33 | D |
| 34 | E |
| 35 | C |
| 36 | D |
| 37 | E |
| 39 | C |
| 41 | A |
| 43 | D |
| 44 | A |
| 45 | A |
| 48 | A |
| 50 | A |
| 51 | A |
| 52 | A |
| 54 | A |
| 56 | A |

*Ki is the dissociation equilibrium constant of the enzyme-inhibitor complex (E-I). Ki values are determined through a series of experiments with varying amounts of inhibitor present. Ki: A ≤ 10 nM; 10 nM < B ≤ 20 nM; 20 nM < C ≤ 50 nM; 50 nM < D ≤ 100 nM; E > 100 nM

### Example C. Cell-Based NanoBRET Target Engagement Assay in 11EK293 Cells Transiently Transfected with LRRK2 WT- or LRRK2 (G2019S)-Nano Luc Fusion Vector

H₁EK293 (ATCC) cells were transfected with 1 g LRRK2 WT or LRRK2 (G2019S) and 9 μg transfection carrier DNA. The transfected cells were treated with the compounds (starting at 1 μM, 10-dose with 3-fold dilution) and reference compounds CEP-701 or Staurosporine (starting at 1 μM, 10-dose with 3-fold dilution); compound treatment time: 1 h. LRRK2 WT and LRRK2 (G2019S) target engagement was measured by NanoBRET assay. Curve fits were performed only when % NanoBret signal at the highest concentration of compounds was less than 5500. Assay format: 384-well format, 4000 cells/well. The IC₅₀ values are shown in Table C, wherein “A” corresponds to IC₅₀≤50.0 nM, “B” corresponds to 50.0 nM≤IC₅₀<100.0 nM, “C” corresponds to 100.0 nM≤IC₅₀.

TABLE C
HEK293 NanoBRET LRRK2[WT] and LRRK2[G2019S]
activities.

| | HEK293 LRRK2[WT] | HEK293 LRRK2[G2019S] |
|---|---|---|
| Compound # | IC50, nM | IC50, nM |
| 2 | C | C |
| 3 | C | C |
| 4 | B | A |
| 14 | C | C |
| 16 | B | A |
| 18 | C | C |
| 19 | B | A |
| 21 | C | B |
| 23 | C | B |
| 26 | C | C |
| 27 | A | A |
| 28 | C | C |
| 30 | C | C |
| 44 | A | A |
| 48 | A | A |
| 49 | A | A |
| 50 | A | A |
| 51 | A | A |
| 52 | A | A |
| 53 | A | A |
| 54 | A | A |
| 55 | A | A |
| 56 | A | A |
| 57 | A | A |
| 59 | A | A |
| 61 | A | A |
| 62 | A | A |
| 63 | A | A |
| 64 | B | A |
| 65 | A | A |
| 67 | C | C |
| 68 | C | C |
| 70 | A | A |
| 73 | B | A |
| 74 | A | A |
| 75 | A | A |
| 76 | A | A |
| 78 | A | A |
| 79 | A | A |
| 81 | A | A |
| 82 | A | A |
| 84 | A | A |
| 85 | A | A |
| 88 | A | A |
| 90 | A | A |
| 92 | A | A |
| 93 | C | C |
| 96 | A | A |

### Example D. A549 Cytotoxicity Assay Used to Determine Safety of the LRRK2 Inhibitors

A549 (ATCC, CCL-185) cells were seeded at a density of 4000 cells per well in a 384-well clear bottom plate (Greiner Cat #781090) in 45 μL total volume of the complete cell culture medium DMEM (PanEco, Cat #C420, Russia) with 10% FBS (HyClone Cat #SV30160.03). The cells were allowed to adhere overnight at 37° C., 5% CO₂. The 500× compounds solutions in DMSO (Sigma Cat #D2650) were prepared in the compounds plate (Diamond Well Plate, Axigen, Cat #P-384-120SQ-C-S) and DMSO as a vehicle control was included. The 1 μL aliquot of 500× compounds (Compounds plate) was added to the 49 μL culture medium in the Dilution plate (Diamond Well Plate, Axigen, Cat #P-384-120SQ-C-S), mixed and then the 5 μL aliquots of the 10× compounds solutions was transferred to cells followed by centrifugation at 100 g for 1 min. Final DMSO concentration was 0.2%. After 3 days of incubation the 10 μL per well of CellTiter-Glo (Promega, CAT #G7572) was added to the cells, plate was centrifuged at 100 g for 1 min. The luminescence signal was detected with CLARIOstar Plus (BMG LABTECH) microplate reader. The CC₅₀ values are shown in Table D.

TABLE D
A549 cytotoxicity.

| Compound # | A549 CC₅₀, μM |
|---|---|
| 2 | >10 |
| 3 | >10 |
| 14 | >10 |
| 27 | >27 |
| 44 | >10 |
| 48 | >10 |
| 50 | >10 |
| 52 | >10 |
| 54 | >10 |
| 56 | >10 |

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A compound of Formula (I): 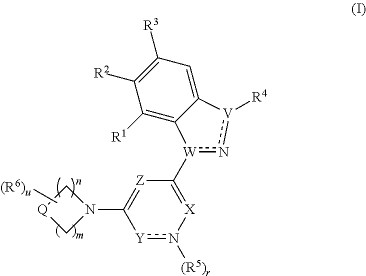
or a pharmaceutically acceptable salt, prodrug, stereoisomer, solvate, or tautomer thereof, wherein
V is selected from C and N;
W is selected from C and N;
and one from V and W is N;
bond V [symbol] N is selected from a single bond and a double bond;
bond W [symbol] N is selected from a single bond and a double bond;
provided that when V is N, the bond V [symbol] N is a single bond, the bond W [symbol] N is a double bond and W is C, and when V is C, the bond V [symbol] N is a double bond, the bond W [symbol] N is a single bond and W is N;
X is selected from CR⁷ and N;
Y is selected from C═O, CR⁸, and N;
provided that one from X and Y is N;
bond Y [symbol] N is selected from a single bond and a double bond;
r is an integer selected from 0 and 1;
provided that when Y is C═O, the bond Y [symbol] N is a single bond and r is 1 and when Y is CR⁸ or N, the bond Y [symbol] N is a double bond and r is 0;
when Y is C═O or N, Z is selected from CR⁹ and N;
when Y is CR⁸, Z is CR⁹;
Q is selected from CH₂, NR⁵, O, S, S(O), S(O)₂
R¹ is selected from H, halogen, C₁-C₆ alkyl, C₁-C₆ alkoxy, C₃-C₁₀ cycloalkyl, C₃-C₁₀ cycloalkoxy, C₂-C₆ alkenyl, C₂-C₆ alkynyl, heterocycle, aryl, heteroaryl, NR¹⁰R¹¹, and S(O)₂R¹² wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl;
R² is selected from H, halogen, C₁-C₆ alkyl, C₁-C₆ alkoxy, C₃-C₁₀ monocyclic cycloalkyl, C₃-C₁₀ cycloalkoxy, C₂-C₆ alkenyl, C₂-C₆ alkynyl, heterocycle, aryl, heteroaryl, C₁-C₆ alkyl-aryl, C₁-C₆ alkyl-heteroaryl, C₂-C₆ alkenyl-aryl, C₂-C₆ alkenyl-heteroaryl, C₂-C₆ alkynyl-aryl, C₂-C₆ alkynyl-heteroaryl, NR¹⁰R¹¹, and S(O)₂R¹² wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl; or
R¹ and R² together with the atoms to which they are attached and any intervening atoms, form a C₃-C₁₀ cycloalkyl, 5-14 membered heterocycle, aryl or heteroaryl wherein the cycloalkyl, heterocycle, aryl or heteroaryl is optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NHC₁-C₆ alkyl, and NR¹⁰R¹¹;
R³ is selected from H, halogen, C₁-C₆ alkyl, C₁-C₆ alkoxy, C₃-C₁₀ monocyclic cycloalkyl, C₃-C₁₀ cycloalkoxy, C₂-C₆ alkenyl, C₂-C₆ alkynyl, heterocycle, aryl, heteroaryl, C₁-C₆ alkyl-aryl, C₁-C₆ alkyl-heteroaryl, C₂-C₆ alkenyl-aryl, C₂-C₆ alkenyl-heteroaryl, C₂-C₆ alkynyl-aryl, C₂-C₆ alkynyl-heteroaryl, NR¹⁰R¹¹, and S(O)₂R¹² wherein the alkyl, alkoxy, alkenyl, alkynyl, heterocycle, cycloalkyl, aryl, or heteroaryl is optionally substituted with one or more substituents independently selected from halogen, OH, NH₂, CN, C₁-C₆ alkyl, C₁-C₆ alkoxy, C₃-C₁₀ cycloalkyl, C₂-C₆ alkenyl, C₂-C₆ alkynyl, heterocycle, aryl, and heteroaryl; or
R² and R³ together with the atoms to which they are attached and any intervening atoms, form a C₃-C₁₀ cycloalkyl, 5-14 membered heterocycle, aryl or heteroaryl wherein the cycloalkyl, heterocycle, aryl or heteroaryl is optionally substituted with one or more substituents independently selected from halogen, OH, oxo, CN, C₁-C₆ alkyl, C₁-C₆ alkoxy, C₁-C₆ alkyl-C₁-C₆ alkoxy, C₁-C₆ alkyl-NHC₁-C₆ alkyl, and NR¹⁰R¹¹;
R⁴ is selected from H, OH, CN, C₁-C₆ alkyl, C₃-C₁₀ cycloalkyl, C₁-C₆ alkyl-C₁-C₆ alkoxy, C₁-C₆ alkyl-NR¹⁰R¹¹, C(O)OC₁-C₆ alkyl, OC(O)C₁-C₆ alkyl wherein the alkyl, cycloalkyl or alkyl-alkoxy is optionally substituted with one or more substituents independently selected from halogen, oxo, OH, C₁-C₆ alkyl, C₁-C₆ alkoxy, NR¹⁰R¹¹, cycloalkyl, and heterocycle;
each R⁵ is independently selected from H, C₁-C₆ alkyl, C₃-C₁₀ cycloalkyl, C₁-C₆ alkyl-C₁-C₆ alkoxy, C₁-C₆ alkyl-NR¹⁰R¹¹, wherein the alkyl, cycloalkyl or alkyl-alkoxy is optionally substituted with one or more substituents independently selected from halogen, oxo, OH, C₁-C₆ alkyl, C₁-C₆ alkoxy, NR¹⁰R¹¹, cycloalkyl, and heterocycle;
each R⁶ is independently selected from halogen, OH, oxo, CN, CONR¹⁰R¹¹, NR¹⁰R¹¹, C₁-C₆ alkyl, C₁-C₆ alkoxy, C₁-C₆ alkyl-C₁-C₆ alkoxy, C₁-C₆ alkyl-C₃-C₈ cycloalkoxy, O—C₁-C₆ alkyl-C(O)NR¹⁰R¹¹, NR¹⁰C(O)R¹¹, —S(O)₂—C₁-C₆ alkyl, —C₁-C₆ alkanediyl-S(O)₂—C₁-C₆ alkyl, wherein the alkyl, or alkoxy is optionally substituted with one or more substituents independently selected from halogen, oxo, OH, C₁-C₆ alkyl, C₁-C₆ alkoxy, NR¹⁰R¹¹, cycloalkyl, and heterocycle;
or two R⁶ together with the atoms to which they are attached and any intervening atoms, form a 3-14 membered cycloalkyl, an aryl, a 3-14 membered heterocycle, or a 5-10 membered heteroaryl, wherein the cycloalkyl, aryl, heterocycle or heteroaryl is optionally substituted with one or more substituents independently selected from halogen, OH, CN, C₁-C₆ alkyl, C₁-C₆ alkoxy, C₁-C₆ hydroxyalkyl, C₁-C₆ alkyl-C₁-C₆ alkoxy, S(O)₂R¹²;
R⁷, R⁸ and R⁹ are each independently selected from H, C₁-C₆ alkyl, C₁-C₆ alkoxy, C₃-C₁₀ cycloalkyl, C₁-C₆ alkyl-C₁-C₆ alkoxy, C₁-C₆ alkyl-NR¹⁰R¹¹, wherein the alkyl, alkoxy, cycloalkyl or alkyl-alkoxy is optionally substituted with one or more substituents independently selected from halogen, oxo, OH, CN, C₁-C₆ alkyl, C₁-C₆ alkoxy, NR¹⁰R¹¹, S(O)₂R¹², cycloalkyl, and heterocycle;
R¹⁰ is selected from H, C₁-C₆ alkyl, C₃-C₁₀ cycloalkyl, S(O)₂R¹², wherein alkyl or cycloalkyl is optionally substituted with one or more substituents independently selected from halogen, oxo, OH, CN, C₁-C₆ alkyl, C₁-C₆ alkoxy;
R¹¹ is selected from H, C₁-C₆ alkyl, C₃-C₁₀ cycloalkyl, wherein alkyl or cycloalkyl is optionally substituted with one or more substituents independently selected from halogen, oxo, OH, CN, C₁-C₆ alkyl, C₁-C₆ alkoxy; or
R¹⁰ and R¹¹ together with the atoms to which they are attached and any intervening atoms, form a 5-14 membered heterocycle, wherein the heterocycle is optionally substituted with one or more substituents independently selected from halogen, OH, CN, C₁-C₆ alkyl, C₁-C₆ alkoxy;
R¹² is selected from C₁-C₆ alkyl, —NH₂, —NH(C₁-C₃ alkyl), or —N(C₁-C₃ alkyl)₂ wherein the alkyl is optionally substituted with one or more substituents independently selected from halogen, oxo, OH, CN, C₁-C₆ alkyl, C₁-C₆ alkoxy;
wherein,
m is an integer selected from 1, 2, 3, 4;
n is an integer selected from 0, 1, 2, 3, 4;
u is an integer selected from 0, 1, 2, 3, 4, 5, 6;
aryl is cyclic, aromatic hydrocarbon groups that have 1 to 3 aromatic rings;
heterocyclyl is saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms selected from O, N, S, P, Se, or B;
heteroaryl is a monovalent monocyclic or a polycyclic aromatic radical of 5 to 24 ring atoms, containing one or more ring heteroatoms selected from N, O, S, P, or B, the remaining ring atoms being C.

2. The compound of claim 1, wherein the compound is of Formula (I-I): 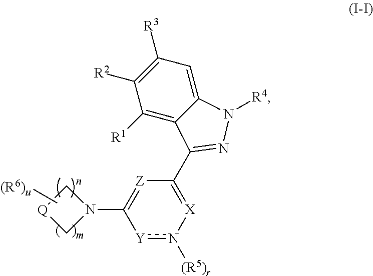
or a pharmaceutically acceptable salt, stereoisomer, solvate, or tautomer thereof.

3. The compound of claim 2, wherein the compound is of Formula (I-I-A), (I-I-B), (I-I-C), or (I-I-D): 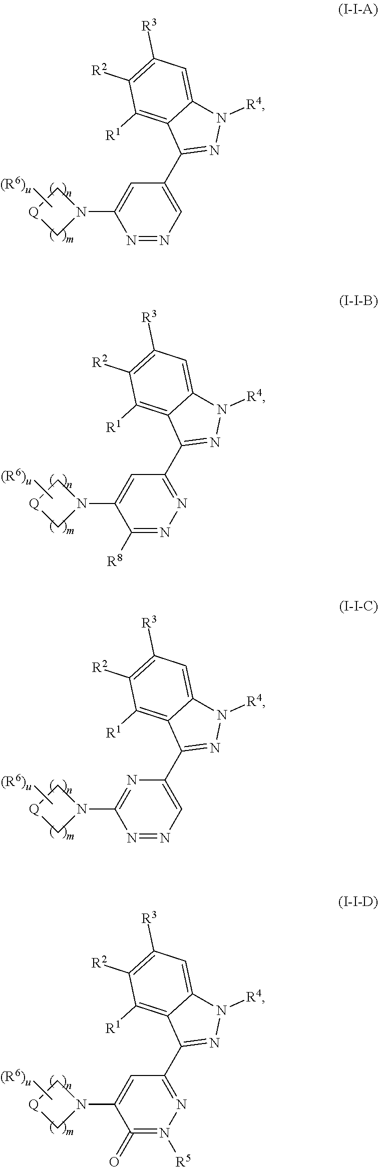
or a pharmaceutically acceptable salt, stereoisomer, solvate, or tautomer thereof.

4. The compound of claim 1, wherein the compound is of Formula (I-II): 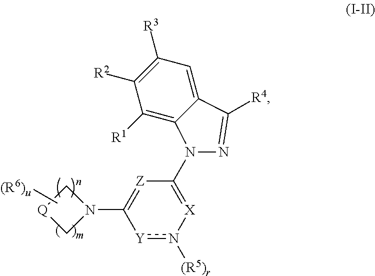
or a pharmaceutically acceptable salt, stereoisomer, solvate, or tautomer thereof.

5. The compound of claim 4, wherein the compound is of Formula (I-II-A), (I-II-B), (I-II-C), or (I-II-D): 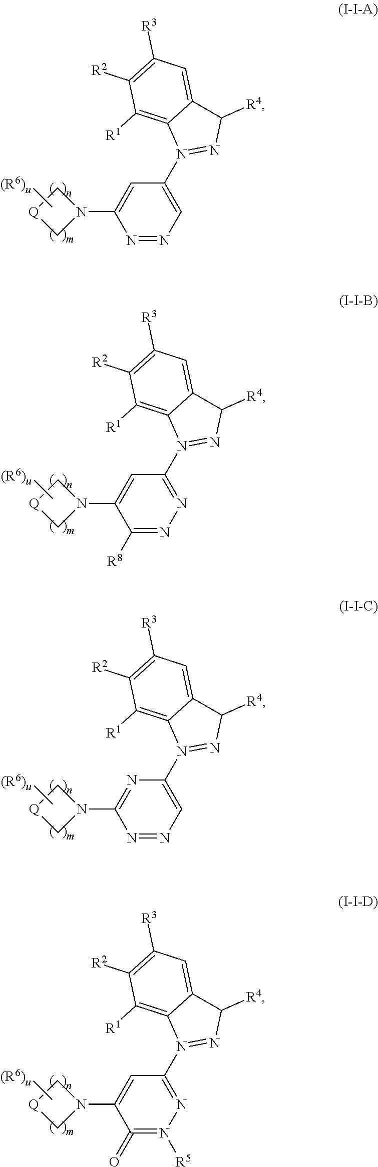
or a pharmaceutically acceptable salt, stereoisomer, solvate, or tautomer thereof.

6. The compound of claim 1, wherein the compound is of Formula (I-O): 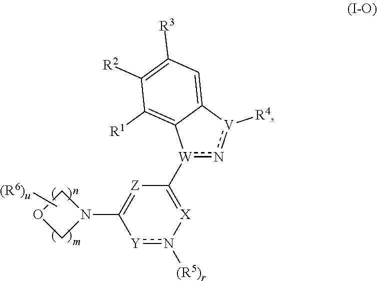
or a pharmaceutically acceptable salt, stereoisomer, solvate, or tautomer thereof.

7. The compound of claim 1, wherein the compound is of Formula (I-N): 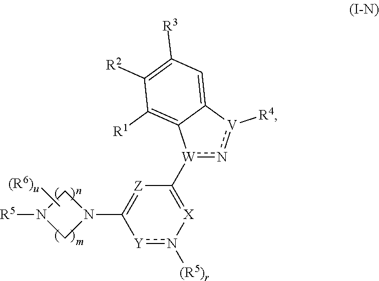
or a pharmaceutically acceptable salt, stereoisomer, solvate, or tautomer thereof.

8. The compound of claim 1, wherein the compound is of Formula (I-C): 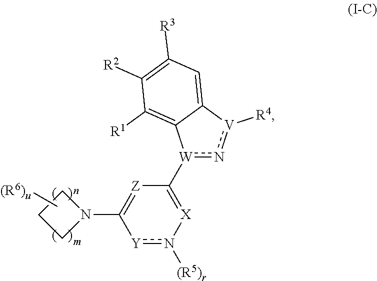
or a pharmaceutically acceptable salt, stereoisomer, solvate, or tautomer thereof.

9. The compound of claim 1, wherein the compound is of Formula (I-S): 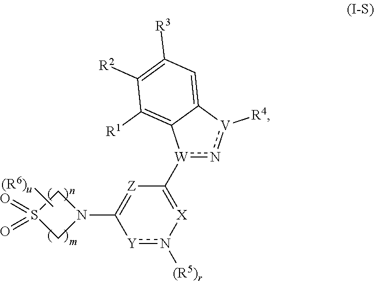
or a pharmaceutically acceptable salt, stereoisomer, solvate, or tautomer thereof.

10. The compound of claim 2, wherein the compound is of Formula (I-O-I): 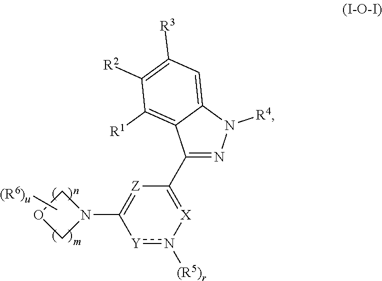
or a pharmaceutically acceptable salt, stereoisomer, solvate, or tautomer thereof.

11. The compound of claim 2, wherein the compound is of Formula (I-N-I): 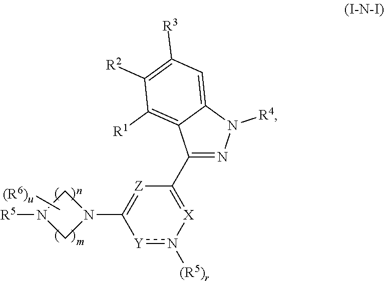
or a pharmaceutically acceptable salt, stereoisomer, solvate, or tautomer thereof.

12. The compound of claim 2, wherein the compound is of Formula (I-C-I): 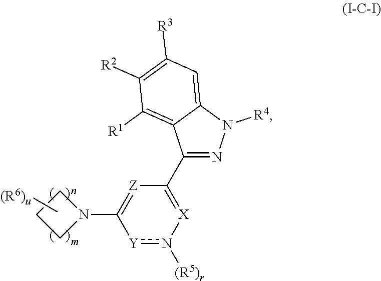
or a pharmaceutically acceptable salt, stereoisomer, solvate, or tautomer thereof.

13. The compound of claim 2, wherein the compound is of Formula (I-S-I): 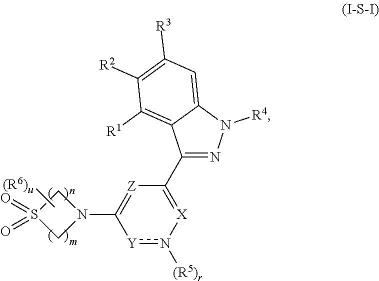
or a pharmaceutically acceptable salt, stereoisomer, solvate, or tautomer thereof.

14. The compound of claim 4, wherein the compound is of Formula (I-O-II): 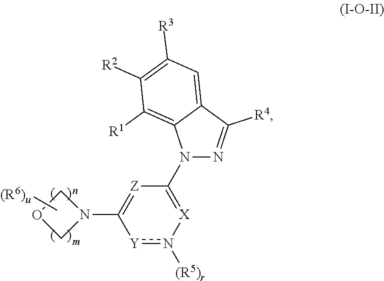
or a pharmaceutically acceptable salt, stereoisomer, solvate, or tautomer thereof.

15. The compound of claim 4, wherein the compound is of Formula (I-N-II): 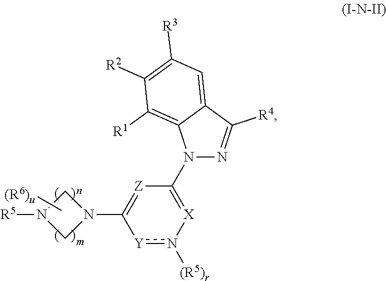
or a pharmaceutically acceptable salt, stereoisomer, solvate, or tautomer thereof.

16. The compound of claim 4, wherein the compound is of Formula (I-C-II): 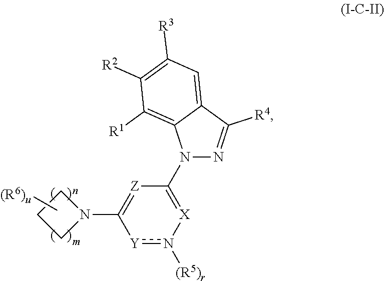
or a pharmaceutically acceptable salt, stereoisomer, solvate, or tautomer thereof.

17. The compound of claim 4, wherein the compound is of Formula (I-S-II): 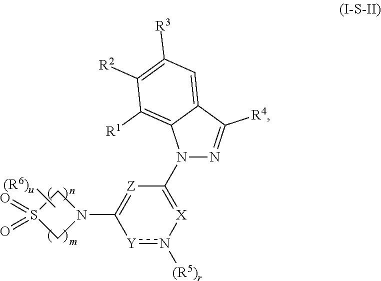
or a pharmaceutically acceptable salt, stereoisomer, solvate, or tautomer thereof.

18. The compound of claim 3, wherein compound is of Formula (I-I-D-I), (I-I-D-II), (I-I-D-III), or (I-I-D-IV): 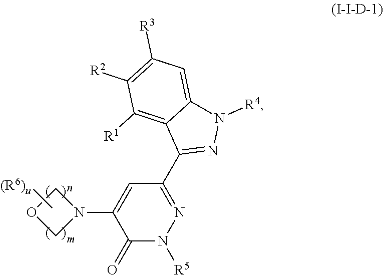 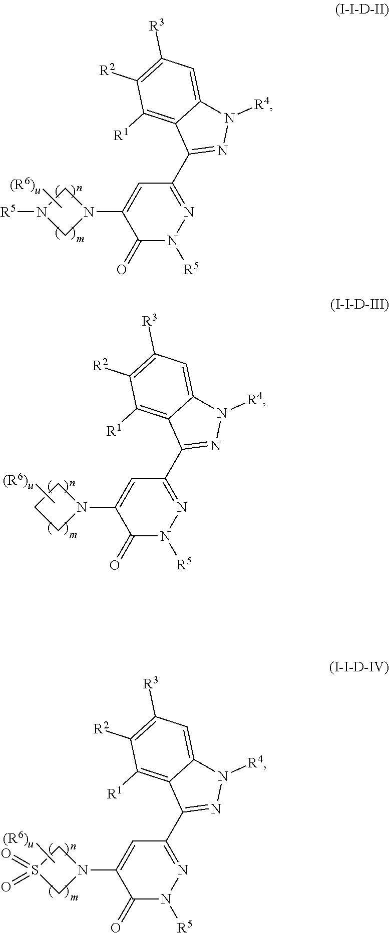
or a pharmaceutically acceptable salt, stereoisomer, solvate, or tautomer thereof.

19. The compound of any one of claims 1, 2, 3, 4, or 5 wherein the fragment 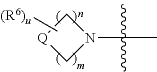
is selected from:

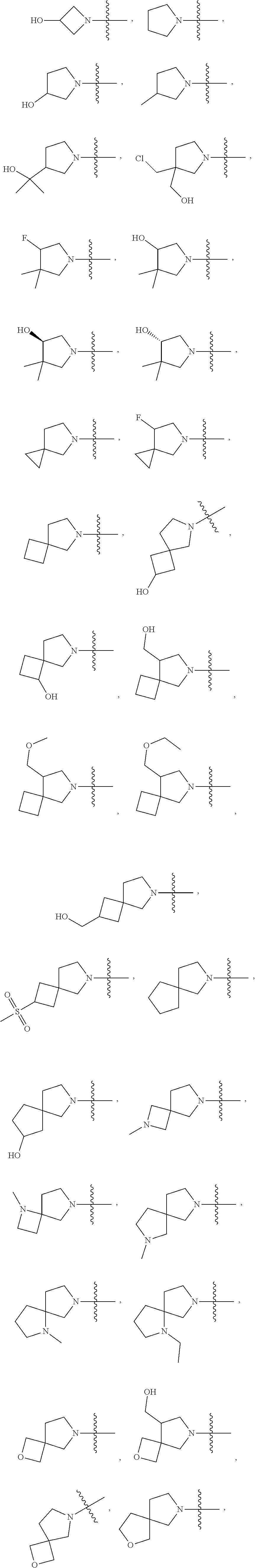

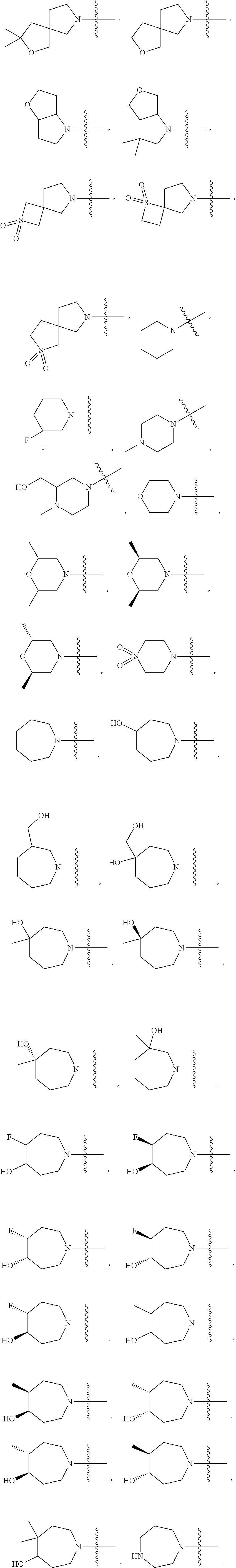

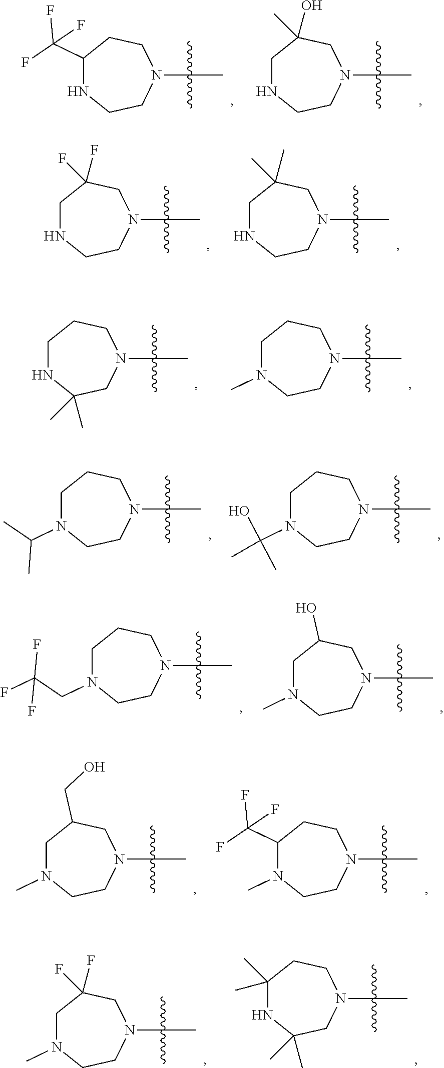

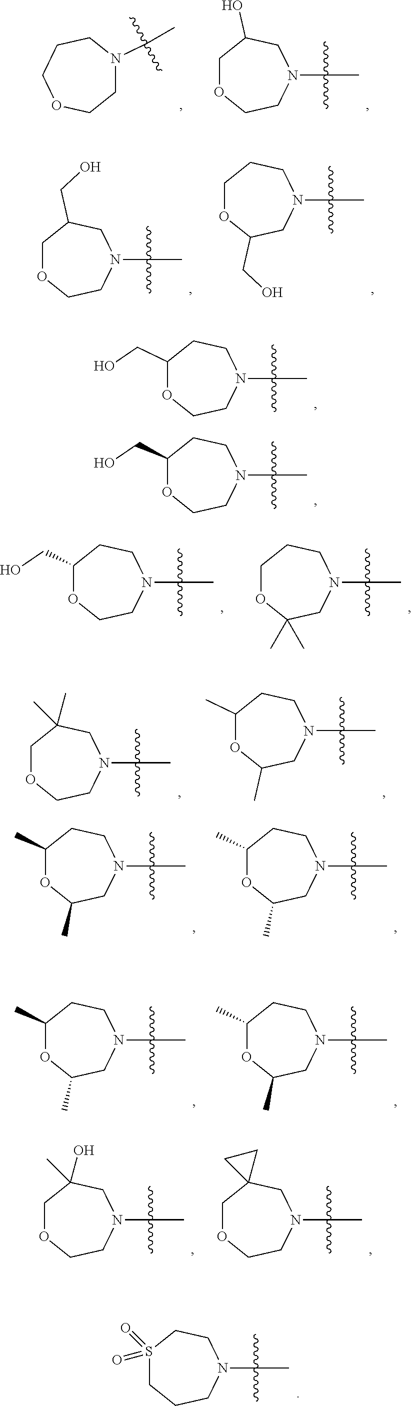

20. The compound of any one of claims 1, 2, 3, 4, or 5, wherein R⁴ is H.

21. A compound is selected from:

| # | Structure | IUPAC Name |
|---|---|---|
| 1 | 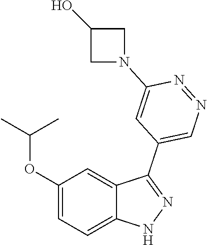 [SMILES: CC(C)Oc1ccc2[nH]nc(-c3cnnc(N4CC(O)C4)c3)c2c1] | 1-[5-(5-isopropoxy-1H-indazol-3- yl)pyridazin-3-yl]azetidin-3-ol |
| 2 | 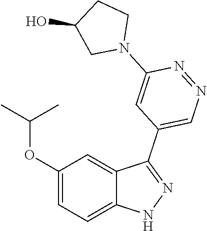 [SMILES: CC(C)Oc1ccc2[nH]nc(-c3cnnc(N4CC[C@H](O)C4)c3)c2c1] | (R)-1-(5-(5-isopropoxy-1H-indazol-3- yl)pyridazin-3-yl)pyrrolidin-3-ol |
| 3 | 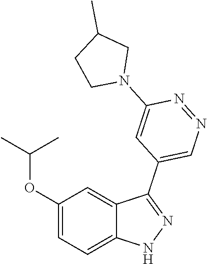 [SMILES: CC1CCN(c2cc(-c3n[nH]c4ccc(OC(C)C)cc34)cnn2)C1] | 5-isopropoxy-3-[6-(3-methylpyrrolidin- 1-yl)pyridazin-4-yl]-1H-indazole |
| 4 | 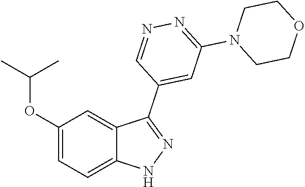 [SMILES: CC(C)Oc1ccc2[nH]nc(-c3cnnc(N4CCOCC4)c3)c2c1] | 4-[5-(5-isopropoxy-1H-indazol-3- yl)pyridazin-3-yl]morpholine |
| 5 | 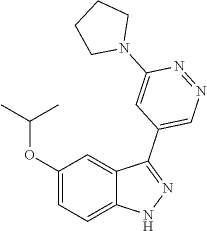 [SMILES: CC(C)Oc1ccc2[nH]nc(-c3cnnc(N4CCCC4)c3)c2c1] | 5-isopropoxy-3-(6-pyrrolidin-1- ylpyridazin-4-yl)-1H-indazole |
| 6 | 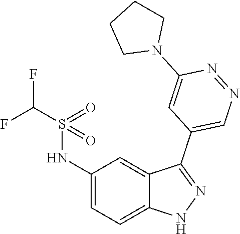 [SMILES: O=S(=O)(Nc1ccc2[nH]nc(-c3cnnc(N4CCCC4)c3)c2c1)C(F)F] | 1,1-difluoro-N-[3-(6-pyrrolidin-1- ylpyridazin-4-yl)-1H-indazol-5- yl]methanesulfonamide |
| 7 | 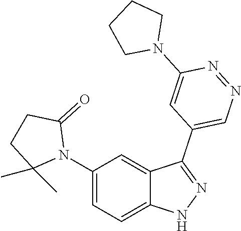 [SMILES: CC1(C)CCC(=O)N1c1ccc2[nH]nc(-c3cnnc(N4CCCC4)c3)c2c1] | 5,5-dimethyl-1-[3-(6-pyrrolidin-1- ylpyridazin-4-yl)-1H-indazol-5- yl]pyrrolidin-2-one |
| 8 | 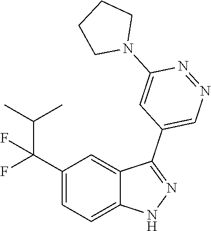 [SMILES: CC(C)C(F)(F)c1ccc2[nH]nc(-c3cnnc(N4CCCC4)c3)c2c1] | 5-(1,1-difluoro-2-methyl-propyl)-3-(6- pyrrolidin-1-ylpyridazin-4-yl)-1H- indazole |
| 9 | 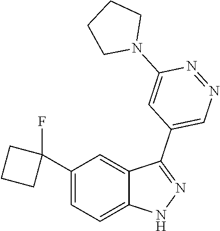 [SMILES: FC1(c2ccc3[nH]nc(-c4cnnc(N5CCCC5)c4)c3c2)CCC1] | 5-(1-fluorocyclobutyl)-3-(6-pyrrolidin-1- ylpyridazin-4-yl)-1H-indazole |
| 10 | 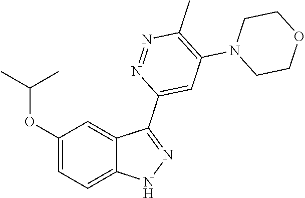 [SMILES: Cc1nnc(-c2n[nH]c3ccc(OC(C)C)cc23)cc1N1CCOCC1] | 4-[6-(5-isopropoxy-1H-indazol-3-yl)-3- methyl-pyridazin-4-yl]morpholine |
| 11 | 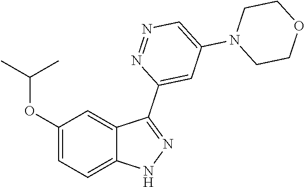 [SMILES: CC(C)Oc1ccc2[nH]nc(-c3cc(N4CCOCC4)cnn3)c2c1] | 4-[6-(5-isopropoxy-1H-indazol-3- yl)pyridazin-4-yl]morpholine |
| 12 | 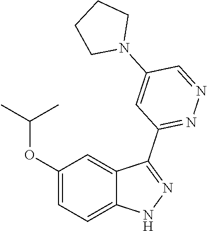 [SMILES: CC(C)Oc1ccc2[nH]nc(-c3cc(N4CCCC4)cnn3)c2c1] | 5-isopropoxy-3-(5-pyrrolidin-1- ylpyridazin-3-yl)-1H-indazole |
| 13 | 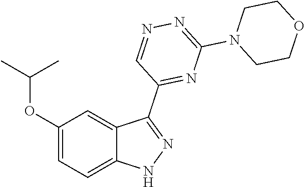 [SMILES: CC(C)Oc1ccc2[nH]nc(-c3cnnc(N4CCOCC4)n3)c2c1] | 4-[5-(5-isopropoxy-1H-indazol-3-yl)- 1,2,4-triazin-3-yl]morpholine |
| 14 | 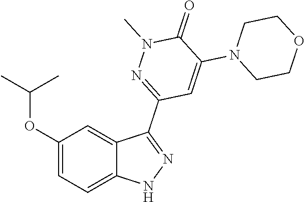 [SMILES: CC(C)Oc1ccc2[nH]nc(-c3cc(N4CCOCC4)c(=O)n(C)n3)c2c1] | 6-(5-isopropoxy-1H-indazol-3-yl)-2- methyl-4-morpholino-pyridazin-3-one |
| 15 | 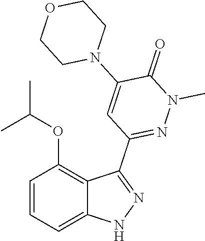 [SMILES: CC(C)Oc1cccc2[nH]nc(-c3cc(N4CCOCC4)c(=O)n(C)n3)c12] | 6-(4-isopropoxy-1H-indazol-3-yl)-2- methyl-4-morpholino-pyridazin-3-one |
| 16 | 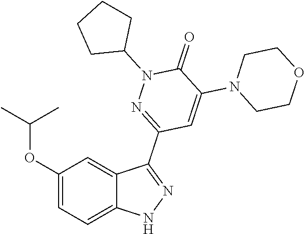 [SMILES: CC(C)Oc1ccc2[nH]nc(-c3cc(N4CCOCC4)c(=O)n(C4CCCC4)n3)c2c1] | 2-cyclopentyl-6-(5-isopropoxy-1H- indazol-3-yl)-4-morpholino-pyridazin-3- one |
| 17 | 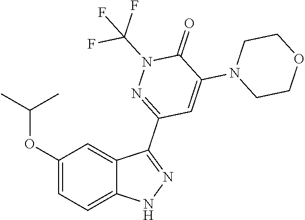 [SMILES: CC(C)Oc1ccc2[nH]nc(-c3cc(N4CCOCC4)c(=O)n(C(F)(F)F)n3)c2c1] | 6-(5-isopropoxy-1H-indazol-3-yl)-4- morpholino-2-(trifluoromethyl)pyridazin- 3-one |
| 18 | 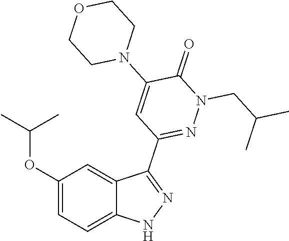 [SMILES: CC(C)Cn1nc(-c2n[nH]c3ccc(OC(C)C)cc23)cc(N2CCOCC2)c1=O] | 2-isobutyl-6-(5-isopropoxy-1H-indazol- 3-yl)-4-morpholino-pyridazin-3-one |
| 19 | 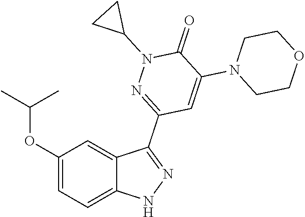 [SMILES: CC(C)Oc1ccc2[nH]nc(-c3cc(N4CCOCC4)c(=O)n(C4CC4)n3)c2c1] | 2-cyclopropyl-6-(5-isopropoxy-1H- indazol-3-yl)-4-morpholino-pyridazin-3- one |
| 20 | 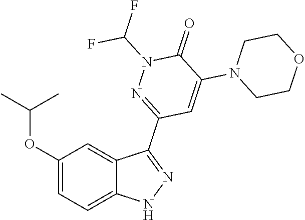 [SMILES: CC(C)Oc1ccc2[nH]nc(-c3cc(N4CCOCC4)c(=O)n(C(F)F)n3)c2c1] | 2-(difluoromethyl)-6-(5-isopropoxy-1H- indazol-3-yl)-4-morpholino-pyridazin-3- one |
| 21 | 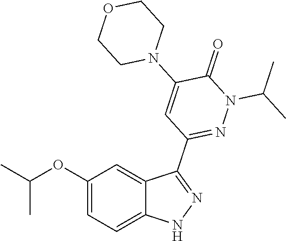 [SMILES: CC(C)Oc1ccc2[nH]nc(-c3cc(N4CCOCC4)c(=O)n(C(C)C)n3)c2c1] | 6-(5-isopropoxy-1H-indazol-3-yl)-2- isopropyl-4-morpholino-pyridazin-3-one |
| 22 | 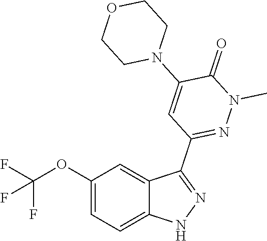 [SMILES: Cn1nc(-c2n[nH]c3ccc(OC(F)(F)F)cc23)cc(N2CCOCC2)c1=O] | 2-methyl-4-morpholino-6-[5- (trifluoromethoxy)-1H-indazol-3- yl]pyridazin-3-one |
| 23 | 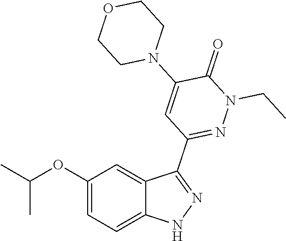 [SMILES: CCn1nc(-c2n[nH]c3ccc(OC(C)C)cc23)cc(N2CCOCC2)c1=O] | 2-ethyl-6-(5-isopropoxy-1H-indazol-3- yl)-4-morpholino-pyridazin-3-one |
| 24 | 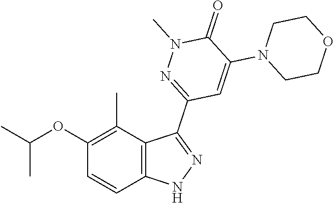 [SMILES: Cc1c(OC(C)C)ccc2[nH]nc(-c3cc(N4CCOCC4)c(=O)n(C)n3)c12] | 6-(5-isopropoxy-4-methyl-1H-indazol-3- yl)-2-methyl-4-morpholino-pyridazin-3- one |
| 25 | 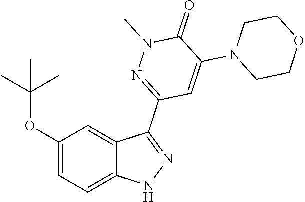 [SMILES: Cn1nc(-c2n[nH]c3ccc(OC(C)(C)C)cc23)cc(N2CCOCC2)c1=O] | 6-(5-tert-butoxy-1H-indazol-3-yl)-2- methyl-4-morpholino-pyridazin-3-one |
| 26 | 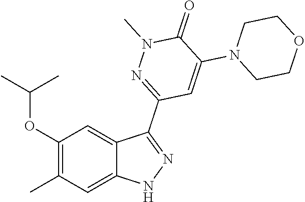 [SMILES: Cc1cc2[nH]nc(-c3cc(N4CCOCC4)c(=O)n(C)n3)c2cc1OC(C)C] | 6-(5-isopropoxy-6-methyl-1H-indazol-3- yl)-2-methyl-4-morpholino-pyridazin-3- one |
| 27 | 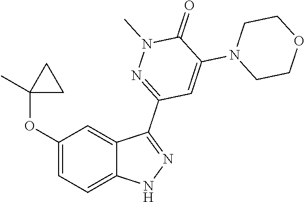 [SMILES: Cn1nc(-c2n[nH]c3ccc(OC4(C)CC4)cc23)cc(N2CCOCC2)c1=O] | 2-methyl-6-[5-(1-methylcyclopropoxy)- 1H-indazol-3-yl]-4-morpholino- pyridazin-3-one |
| 28 | 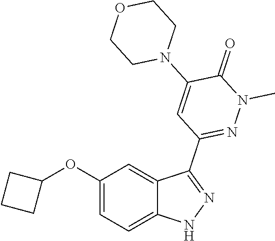 [SMILES: Cn1nc(-c2n[nH]c3ccc(OC4CCC4)cc23)cc(N2CCOCC2)c1=O] | 6-[5-(cyclobutoxy)-1H-indazol-3-yl]-2- methyl-4-morpholino-pyridazin-3-one |
| 29 | 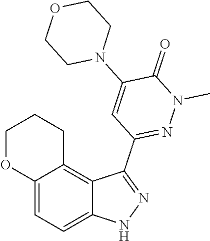 [SMILES: Cn1nc(-c2n[nH]c3ccc4c(c23)CCCO4)cc(N2CCOCC2)c1=O] | 2-methyl-4-morpholino-6-(3,7,8,9- tetrahydropyrano[3,2-e]indazol-1- yl)pyridazin-3-one |
| 30 | 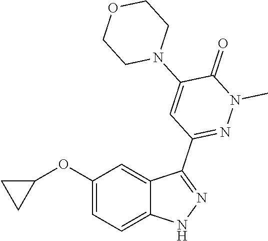 [SMILES: Cn1nc(-c2n[nH]c3ccc(OC4CC4)cc23)cc(N2CCOCC2)c1=O] | 6-[5-(cyclopropoxy)-1H-indazol-3-yl]-2- methyl-4-morpholino-pyridazin-3-one |
| 31 | 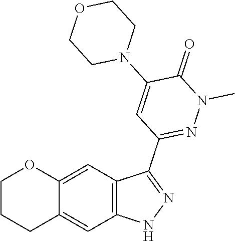 [SMILES: Cn1nc(-c2n[nH]c3cc4c(cc23)OCCC4)cc(N2CCOCC2)c1=O] | 2-methyl-4-morpholino-6-(1,6,7,8- tetrahydropyrano[2,3-f]indazol-3- yl)pyridazin-3-one |
| 32 | 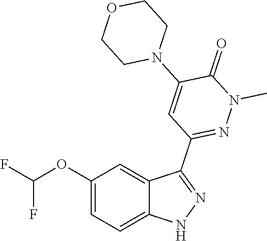 [SMILES: Cn1nc(-c2n[nH]c3ccc(OC(F)F)cc23)cc(N2CCOCC2)c1=O] | 6-[5-(difluoromethoxy)-1H-indazol-3- yl]-2-methyl-4-morpholino-pyridazin-3- one |
| 33 | 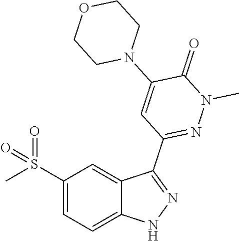 | 2-methyl-6-(5-methylsulfonyl-1H- indazol-3-yl)-4-morpholino-pyridazin-3- one |
| 34 | 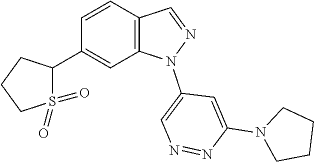 [SMILES: O=S1(=O)CCCC1c1ccc2cnn(-c3cnnc(N4CCCC4)c3)c2c1] | 2-[1-(6-pyrrolidin-1-ylpyridazin-4- yl)indazol-6-yl]thiolane 1,1-dioxide |
| 35 | 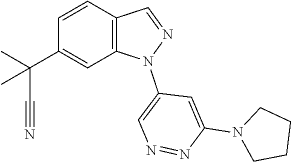 [SMILES: CC(C)(C#N)c1ccc2cnn(-c3cnnc(N4CCCC4)c3)c2c1] | 2-methyl-2-[1-(6-pyrrolidin-1- ylpyridazin-4-yl)indazol-6- yl]propanenitrile |
| 36 | 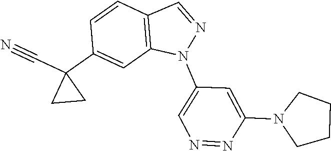 [SMILES: N#CC1(c2ccc3cnn(-c4cnnc(N5CCCC5)c4)c3c2)CC1] | 1-[1-(6-pyrrolidin-1-ylpyridazin-4- yl)indazol-6-yl]cyclopropanecarbonitrile |
| 37 | 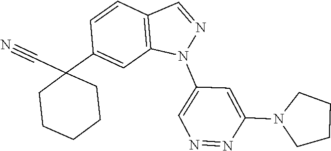 [SMILES: N#CC1(c2ccc3cnn(-c4cnnc(N5CCCC5)c4)c3c2)CCCCC1] | 1-[1-(6-pyrrolidin-1-ylpyridazin-4- yl)indazol-6-yl]cyclohexanecarbonitrile |
| 38 | 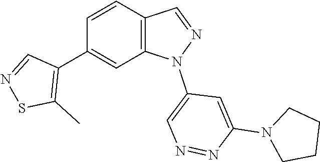 [SMILES: Cc1sncc1-c1ccc2cnn(-c3cnnc(N4CCCC4)c3)c2c1] | 5-methyl-4-[1-(6-pyrrolidin-1- ylpyridazin-4-yl)indazol-6-yl]isothiazole |
| 39 | 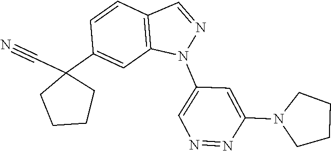 [SMILES: N#CC1(c2ccc3cnn(-c4cnnc(N5CCCC5)c4)c3c2)CCCC1] | 1-[1-(6-pyrrolidin-1-ylpyridazin-4- yl)indazol-6-yl]cyclopentanecarbonitrile |
| 40 | 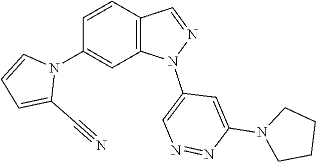 [SMILES: N#Cc1cccn1-c1ccc2cnn(-c3cnnc(N4CCCC4)c3)c2c1] | 1-[1-(6-pyrrolidin-1-ylpyridazin-4- yl)indazol-6-yl]pyrrole-2-carbonitrile |
| 41 | 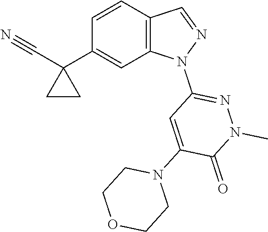 [SMILES: Cn1nc(-n2ncc3ccc(C4(C#N)CC4)cc32)cc(N2CCOCC2)c1=O] | 1-[1-(1-methyl-5-morpholino-6-oxo- pyridazin-3-yl)indazol-6- yl]cyclopropanecarbonitrile |
| 42 | 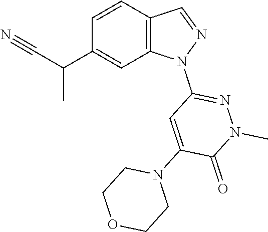 [SMILES: CC(C#N)c1ccc2cnn(-c3cc(N4CCOCC4)c(=O)n(C)n3)c2c1] | 2-[1-(1-methyl-5-morpholino-6-oxo- pyridazin-3-yl)indazol-6- yl]propanenitrile |
| 43 | 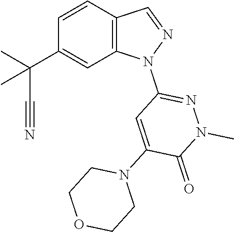 [SMILES: Cn1nc(-n2ncc3ccc(C(C)(C)C#N)cc32)cc(N2CCOCC2)c1=O] | 2-methyl-2-[1-(1-methyl-5-morpholino- 6-oxo-pyridazin-3-yl)indazol-6- yl]propanenitrile |
| 44 | 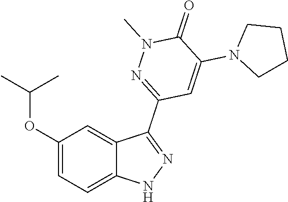 [SMILES: CC(C)Oc1ccc2[nH]nc(-c3cc(N4CCCC4)c(=O)n(C)n3)c2c1] | 6-(5-isopropoxy-1H-indazol-3-yl)-2- methyl-4-pyrrolidin-1-yl-pyridazin-3-one |
| 45 | 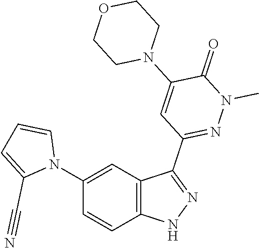 [SMILES: Cn1nc(-c2n[nH]c3ccc(-n4cccc4C#N)cc23)cc(N2CCOCC2)c1=O] | 1-[3-(1-methyl-5-morpholino-6-oxo- pyridazin-3-yl)-1H-indazol-5-yl]pyrrole- 2-carbonitrile |
| 46 | 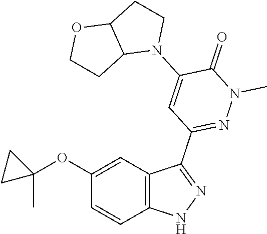 [SMILES: Cn1nc(-c2n[nH]c3ccc(OC4(C)CC4)cc23)cc(N2CCC3OCCC32)c1=O] | 4-(2,3,3a,5,6,6a-hexahydrofuro[3,2- b]pyrrol-4-yl)-2-methyl-6-[5-(1- methylcyclopropoxy)-1H-indazol-3- yl]pyridazin-3-one |
| 47 | 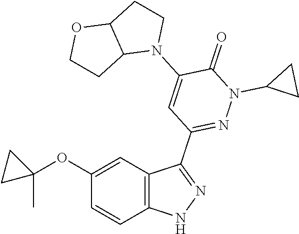 [SMILES: CC1(Oc2ccc3[nH]nc(-c4cc(N5CCC6OCCC65)c(=O)n(C5CC5)n4)c3c2)CC1] | 4-(2,3,3a,5,6,6a-hexahydrofuro[3,2- b]pyrrol-4-yl)-2-cyclopropyl-6-[5-(1- methylcyclopropoxy)-1H-indazol-3- yl]pyridazin-3-one |
| 48 | 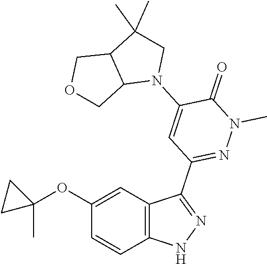 [SMILES: Cn1nc(-c2n[nH]c3ccc(OC4(C)CC4)cc23)cc(N2CC(C)(C)C3COCC32)c1=O] | 4-(3,3-dimethyl-3a,4,6,6a-tetrahydro-2H- furo[3,4-b]pyrrol-1-yl)-2-methyl-6-[5-(1- methylcyclopropoxy)-1H-indazol-3- yl]pyridazin-3-one |
| 49 | 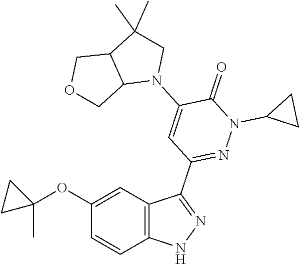 [SMILES: CC1(Oc2ccc3[nH]nc(-c4cc(N5CC(C)(C)C6COCC65)c(=O)n(C5CC5)n4)c3c2)CC1] | 2-cyclopropyl-4-(3,3-dimethyl-3a,4,6,6a- tetrahydro-2H-furo[3,4-b]pyrrol-1-yl)-6- [5-(1-methylcyclopropoxy)-1H-indazol- 3-yl]pyridazin-3-one |
| 50 | 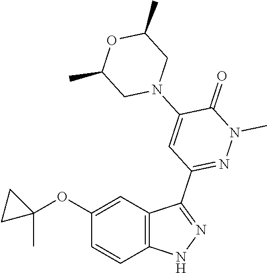 [SMILES: C[C@@H]1CN(c2cc(-c3n[nH]c4ccc(OC5(C)CC5)cc34)nn(C)c2=O)C[C@H](C)O1] | 4-[(2S,6R)-2,6-dimethylmorpholin-4-yl]- 2-methyl-6-[5-(1-methylcyclopropoxy)- 1H-indazol-3-yl]pyridazin-3-one |
| 51 | 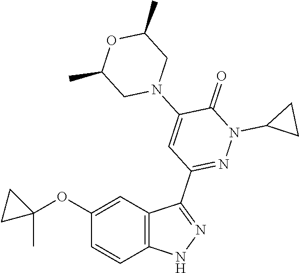 [SMILES: C[C@@H]1CN(c2cc(-c3n[nH]c4ccc(OC5(C)CC5)cc34)nn(C3CC3)c2=O)C[C@H](C)O1] | 2-cyclopropyl-4-[(2S,6R)-2,6- dimethylmorpholin-4-yl]-6-[5-(1- methylcyclopropoxy)-1H-indazol-3- yl]pyridazin-3-one |
| 52 | 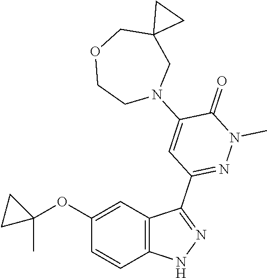 [SMILES: Cn1nc(-c2n[nH]c3ccc(OC4(C)CC4)cc23)cc(N2CCOCC3(CC3)C2)c1=O] | 2-methyl-6-[5-(1-methylcyclopropoxy)- 1H-indazol-3-yl]-4-(5-oxa-8- azaspiro[2.6]nonan-8-yl)pyridazin-3-one |
| 53 | 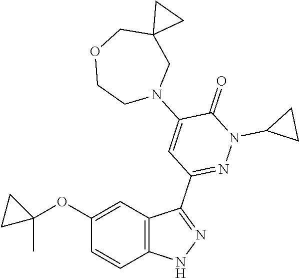 [SMILES: CC1(Oc2ccc3[nH]nc(-c4cc(N5CCOCC6(CC6)C5)c(=O)n(C5CC5)n4)c3c2)CC1] | 2-cyclopropyl-6-[5-(1- methylcyclopropoxy)-1H-indazol-3-yl]- 4-(5-oxa-8-azaspiro[2.6]nonan-8- yl)pyridazin-3-one |
| 54 | 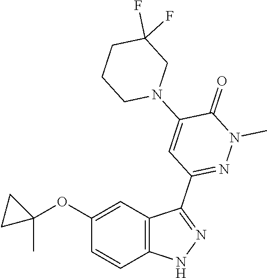 [SMILES: Cn1nc(-c2n[nH]c3ccc(OC4(C)CC4)cc23)cc(N2CCCC(F)(F)C2)c1=O] | 4-(3,3-difluoro-1-piperidyl)-2-methyl-6- [5-(1-methylcyclopropoxy)-1H-indazol- 3-yl]pyridazin-3-one |
| 55 | 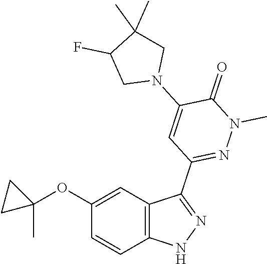 [SMILES: Cn1nc(-c2n[nH]c3ccc(OC4(C)CC4)cc23)cc(N2CC(F)C(C)(C)C2)c1=O] | 4-(4-fluoro-3,3-dimethyl-pyrrolidin-1- yl)-2-methyl-6-[5-(1- methylcyclopropoxy)-1H-indazol-3- yl]pyridazin-3-one |
| 56 | 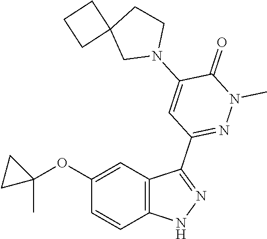 [SMILES: Cn1nc(-c2n[nH]c3ccc(OC4(C)CC4)cc23)cc(N2CCC3(CCC3)C2)c1=O] | 4-(6-azaspiro[3.4]octan-6-yl)-2-methyl- 6-[5-(1-methylcyclopropoxy)-1H- indazol-3-yl]pyridazin-3-one |
| 57 | 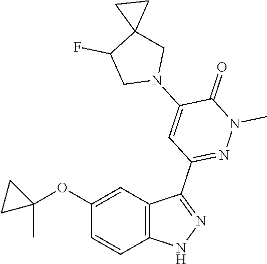 [SMILES: Cn1nc(-c2n[nH]c3ccc(OC4(C)CC4)cc23)cc(N2CC(F)C3(CC3)C2)c1=O] | 4-(7-fluoro-5-azaspiro[2.4]heptan-5-yl)- 2-methyl-6-[5-(1-methylcyclopropoxy)- 1H-indazol-3-yl]pyridazin-3-one |
| 58 | 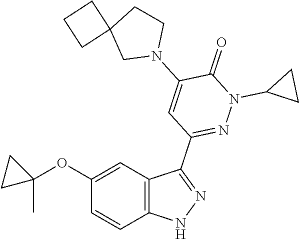 [SMILES: CC1(Oc2ccc3[nH]nc(-c4cc(N5CCC6(CCC6)C5)c(=O)n(C5CC5)n4)c3c2)CC1] | 4-(6-azaspiro[3.4]octan-6-yl)-2- cyclopropyl-6-[5-(1- methylcyclopropoxy)-1H-indazol-3- yl]pyridazin-3-one |
| 59 | 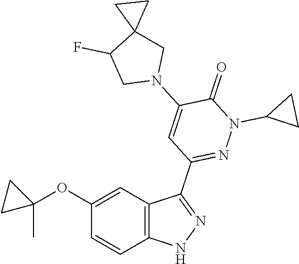 [SMILES: CC1(Oc2ccc3[nH]nc(-c4cc(N5CC(F)C6(CC6)C5)c(=O)n(C5CC5)n4)c3c2)CC1] | 2-cyclopropyl-4-(7-fluoro-5- azaspiro[2.4]heptan-5-yl)-6-[5-(1- methylcyclopropoxy)-1H-indazol-3- yl]pyridazin-3-one |
| 60 | 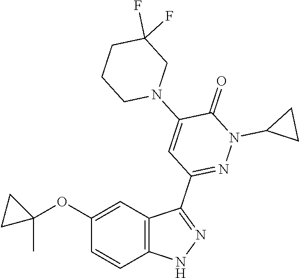 [SMILES: CC1(Oc2ccc3[nH]nc(-c4cc(N5CCCC(F)(F)C5)c(=O)n(C5CC5)n4)c3c2)CC1] | 2-cyclopropyl-4-(3,3-difluoro-1- piperidyl)-6-[5-(1-methylcyclopropoxy)- 1H-indazol-3-yl]pyridazin-3-one |
| 61 | 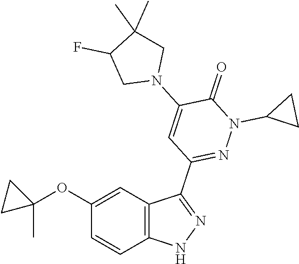 [SMILES: CC1(Oc2ccc3[nH]nc(-c4cc(N5CC(F)C(C)(C)C5)c(=O)n(C5CC5)n4)c3c2)CC1] | 2-cyclopropyl-4-(4-fluoro-3,3-dimethyl- pyrrolidin-1-yl)-6-[5-(1- methylcyclopropoxy)-1H-indazol-3- yl]pyridazin-3-one |
| 62 | 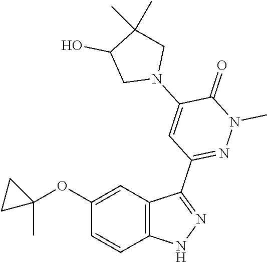 [SMILES: Cn1nc(-c2n[nH]c3ccc(OC4(C)CC4)cc23)cc(N2CC(O)C(C)(C)C2)c1=O] | 4-(4-hydroxy-3,3-dimethyl-pyrrolidin-1- yl)-2-methyl-6-[5-(1- methylcyclopropoxy)-1H-indazol-3- yl]pyridazin-3-one |
| 63 | 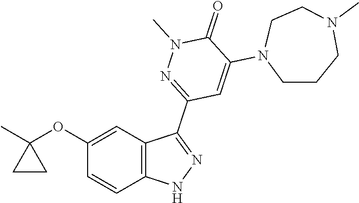 | 2-methyl-6-[5-(1-methylcyclopropoxy)- 1H-indazol-3-yl]-4-(4-methyl-1,4- diazepan-1-yl)pyridazin-3-one |
| 64 | 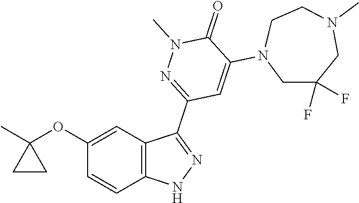 | 4-(6,6-difluoro-4-methyl-1,4-diazepan-1- yl)-2-methyl-6-[5-(1- methylcyclopropoxy)-1H-indazol-3- yl]pyridazin-3-one |
| 65 | 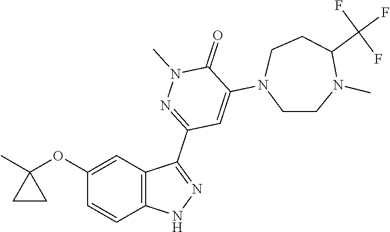 [SMILES: CN1CCN(c2cc(-c3n[nH]c4ccc(OC5(C)CC5)cc34)nn(C)c2=O)CCC1C(F)(F)F] | 2-methyl-6-[5-(1-methylcyclopropoxy)- 1H-indazol-3-yl]-4-[4-methyl-5- (trifluoromethyl)-1,4-diazepan-1- yl]pyridazin-3-one |
| 66 | 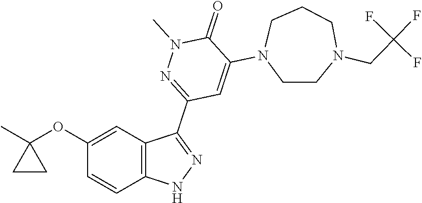 [SMILES: Cn1nc(-c2n[nH]c3ccc(OC4(C)CC4)cc23)cc(N2CCCN(CC(F)(F)F)CC2)c1=O] | 2-methyl-6-[5-(1-methylcyclopropoxy)- 1H-indazol-3-yl]-4-[4-(2,2,2- trifluoroethyl)-1,4-diazepan-1- yl]pyridazin-3-one |
| 67 | 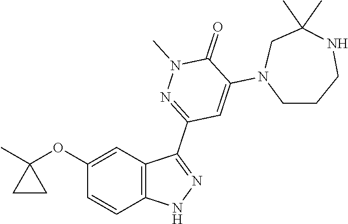 [SMILES: Cn1nc(-c2n[nH]c3ccc(OC4(C)CC4)cc23)cc(N2CCCNC(C)(C)C2)c1=O] | 4-(3,3-dimethyl-1,4-diazepan-1-yl)-2- methyl-6-[5-(1-methylcyclopropoxy)- 1H-indazol-3-yl]pyridazin-3-one |
| 68 | 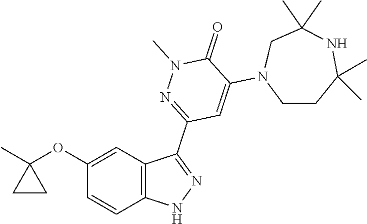 | 2-methyl-6-[5-(1-methylcyclopropoxy)- 1H-indazol-3-yl]-4-(3,3,5,5-tetramethyl- 1,4-diazepan-1-yl)pyridazin-3-one |
| 69 | 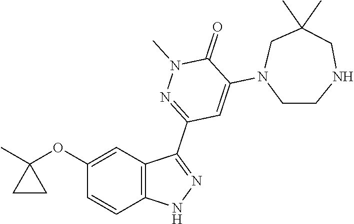 [SMILES: Cn1nc(-c2n[nH]c3ccc(OC4(C)CC4)cc23)cc(N2CCNCC(C)(C)C2)c1=O] | 4-(6,6-dimethyl-1,4-diazepan-1-yl)-2- methyl-6-[5-(1-methylcyclopropoxy)- 1H-indazol-3-yl]pyridazin-3-one |
| 70 | 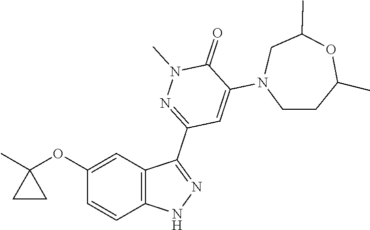 | 4-(2,7-dimethyl-1,4-oxazepan-4-yl)-2- methyl-6-[5-(1-methylcyclopropoxy)- 1H-indazol-3-yl]pyridazin-3-one |
| 71 | 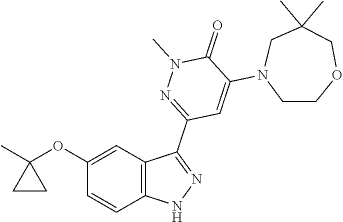 [SMILES: Cn1nc(-c2n[nH]c3ccc(OC4(C)CC4)cc23)cc(N2CCOCC(C)(C)C2)c1=O] | 4-(6,6-dimethyl-1,4-oxazepan-4-yl)-2- methyl-6-[5-(1-methylcyclopropoxy)- 1H-indazol-3-yl]pyridazin-3-one |
| 72 | 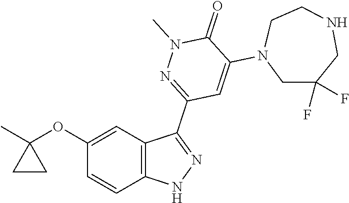 [SMILES: Cn1nc(-c2n[nH]c3ccc(OC4(C)CC4)cc23)cc(N2CCNCC(F)(F)C2)c1=O] | 4-(6,6-difluoro-1,4-diazepan-1-yl)-2- methyl-6-[5-(1-methylcyclopropoxy)- 1H-indazol-3-yl]pyridazin-3-one |
| 73 | 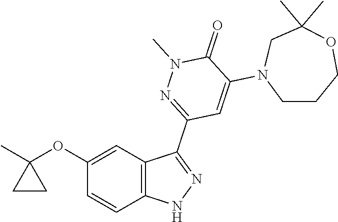 [SMILES: Cn1nc(-c2n[nH]c3ccc(OC4(C)CC4)cc23)cc(N2CCCOC(C)(C)C2)c1=O] | 4-(2,2-dimethyl-1,4-oxazepan-4-yl)-2- methyl-6-[5-(1-methylcyclopropoxy)- 1H-indazol-3-yl]pyridazin-3-one |
| 74 | 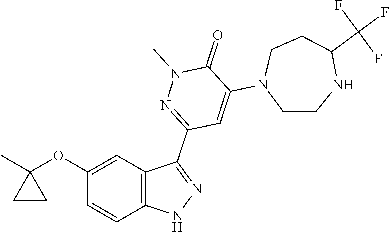 [SMILES: Cn1nc(-c2n[nH]c3ccc(OC4(C)CC4)cc23)cc(N2CCNC(C(F)(F)F)CC2)c1=O] | 2-methyl-6-[5-(1-methylcyclopropoxy)- 1H-indazol-3-yl]-4-[5-(trifluoromethyl)- 1,4-diazepan-1-yl]pyridazin-3-one |
| 75 | 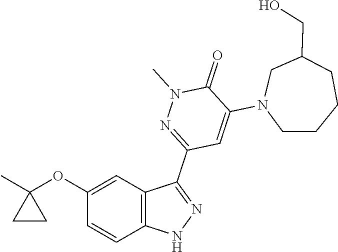 [SMILES: Cn1nc(-c2n[nH]c3ccc(OC4(C)CC4)cc23)cc(N2CCCCC(CO)C2)c1=O] | 4-[3-(hydroxymethyl)azepan-1-yl]-2- methyl-6-[5-(1-methylcyclopropoxy)- 1H-indazol-3-yl]pyridazin-3-one |
| 76 | 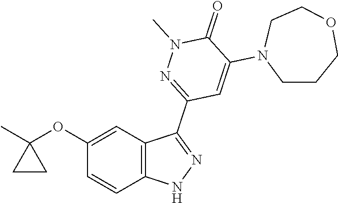 [SMILES: Cn1nc(-c2n[nH]c3ccc(OC4(C)CC4)cc23)cc(N2CCCOCC2)c1=O] | 2-methyl-6-[5-(1-methylcyclopropoxy)- 1H-indazol-3-yl]-4-(1,4-oxazepan-4- yl)pyridazin-3-one |
| 77 | 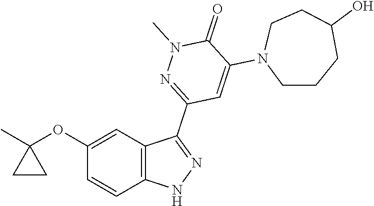 [SMILES: Cn1nc(-c2n[nH]c3ccc(OC4(C)CC4)cc23)cc(N2CCCC(O)CC2)c1=O] | 4-(4-hydroxyazepan-1-yl)-2-methyl-6-[5- (1-methylcyclopropoxy)-1H-indazol-3- yl]pyridazin-3-one |
| 78 | 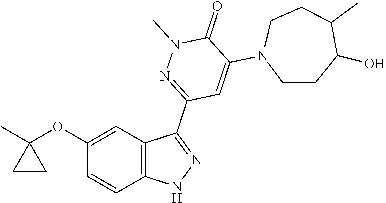 [SMILES: CC1CCN(c2cc(-c3n[nH]c4ccc(OC5(C)CC5)cc34)nn(C)c2=O)CCC1O] | 4-(4-hydroxy-5-methyl-azepan-1-yl)-2- methyl-6-[5-(1-methylcyclopropoxy)- 1H-indazol-3-yl]pyridazin-3-one |
| 79 | 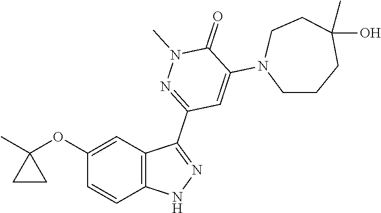 [SMILES: Cn1nc(-c2n[nH]c3ccc(OC4(C)CC4)cc23)cc(N2CCCC(C)(O)CC2)c1=O] | 4-(4-hydroxy-4-methyl-azepan-1-yl)-2- methyl-6-[5-(1-methylcyclopropoxy)- 1H-indazol-3-yl]pyridazin-3-one |
| 80 | 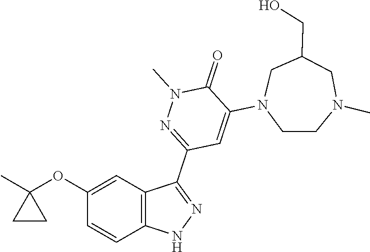 | 4-[6-(hydroxymethyl)-4-methyl-1,4- diazepan-1-yl]-2-methyl-6-[5-(1- methylcyclopropoxy)-1H-indazol-3- yl]pyridazin-3-one |
| 81 | 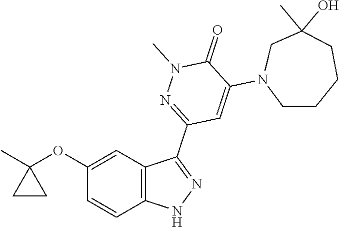 [SMILES: Cn1nc(-c2n[nH]c3ccc(OC4(C)CC4)cc23)cc(N2CCCCC(C)(O)C2)c1=O] | 4-(3-hydroxy-3-methyl-azepan-1-yl)-2- methyl-6-[5-(1-methylcyclopropoxy)- 1H-indazol-3-yl]pyridazin-3-one |
| 82 | 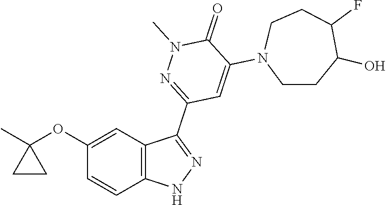 [SMILES: Cn1nc(-c2n[nH]c3ccc(OC4(C)CC4)cc23)cc(N2CCC(O)C(F)CC2)c1=O] | 4-(4-fluoro-5-hydroxy-azepan-1-yl)-2- methyl-6-[5-(1-methylcyclopropoxy)- 1H-indazol-3-yl]pyridazin-3-one |
| 83 | 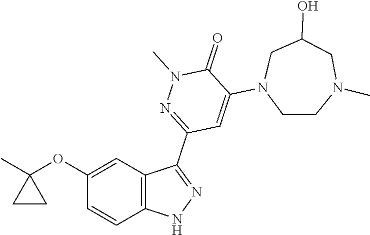 | 4-(6-hydroxy-4-methyl-1,4-diazepan-1- yl)-2-methyl-6-[5-(1- methylcyclopropoxy)-1H-indazol-3- yl]pyridazin-3-one |
| 84 | 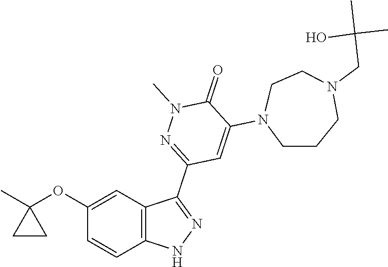 [SMILES: Cn1nc(-c2n[nH]c3ccc(OC4(C)CC4)cc23)cc(N2CCCN(CC(C)(C)O)CC2)c1=O] | 4-[4-(2-hydroxy-2-methyl-propyl)-1,4- diazepan-1-yl]-2-methyl-6-[5-(1- methylcyclopropoxy)-1H-indazol-3- yl]pyridazin-3-one |
| 85 | 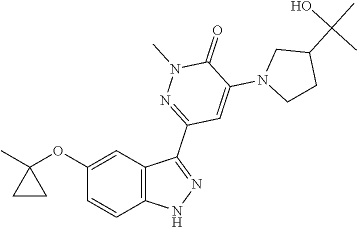 [SMILES: Cn1nc(-c2n[nH]c3ccc(OC4(C)CC4)cc23)cc(N2CCC(C(C)(C)O)C2)c1=O] | 4-[3-(1-hydroxy-1-methyl- ethyl)pyrrolidin-1-yl]-2-methyl-6-[5-(1- methylcyclopropoxy)-1H-indazol-3- yl]pyridazin-3-one |
| 86 | 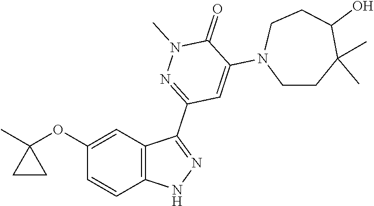 | 4-(5-hydroxy-4,4-dimethyl-azepan-1-yl)- 2-methyl-6-[5-(1-methylcyclopropoxy)- 1H-indazol-3-yl]pyridazin-3-one |
| 87 | 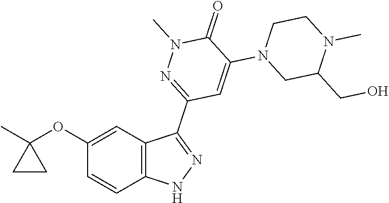 [SMILES: CN1CCN(c2cc(-c3n[nH]c4ccc(OC5(C)CC5)cc34)nn(C)c2=O)CC1CO] | 4-[3-(hydroxymethyl)-4-methyl- piperazin-1-yl]-2-methyl-6-[5-(1- methylcyclopropoxy)-1H-indazol-3- yl]pyridazin-3-one |
| 88 | 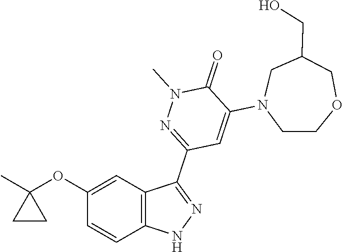 [SMILES: Cn1nc(-c2n[nH]c3ccc(OC4(C)CC4)cc23)cc(N2CCOCC(CO)C2)c1=O] | 4-[6-(hydroxymethyl)-1,4-oxazepan-4- yl]-2-methyl-6-[5-(1- methylcyclopropoxy)-1H-indazol-3- yl]pyridazin-3-one |
| 89 | 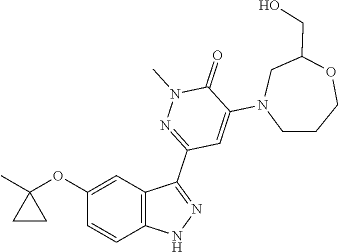 [SMILES: Cn1nc(-c2n[nH]c3ccc(OC4(C)CC4)cc23)cc(N2CCCOC(CO)C2)c1=O] | 4-[2-(hydroxymethyl)-1,4-oxazepan-4- yl]-2-methyl-6-[5-(1- methylcyclopropoxy)-1H-indazol-3- yl]pyridazin-3-one |
| 90 | 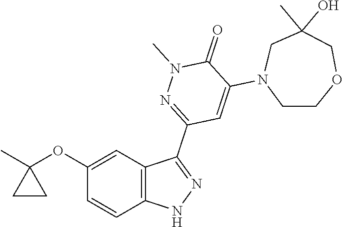 [SMILES: Cn1nc(-c2n[nH]c3ccc(OC4(C)CC4)cc23)cc(N2CCOCC(C)(O)C2)c1=O] | 4-(6-hydroxy-6-methyl-1,4-oxazepan-4- yl)-2-methyl-6-[5-(1- methylcyclopropoxy)-1H-indazol-3- yl]pyridazin-3-one |
| 91 | 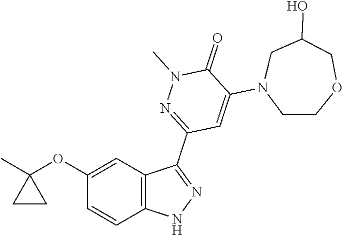 [SMILES: Cn1nc(-c2n[nH]c3ccc(OC4(C)CC4)cc23)cc(N2CCOCC(O)C2)c1=O] | 4-(6-hydroxy-1,4-oxazepan-4-yl)-2- methyl-6-[5-(1-methylcyclopropoxy)- 1H-indazol-3-yl]pyridazin-3-one |
| 92 | 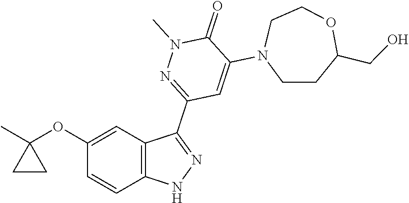 [SMILES: Cn1nc(-c2n[nH]c3ccc(OC4(C)CC4)cc23)cc(N2CCOC(CO)CC2)c1=O] | 4-[7-(hydroxymethyl)-1,4-oxazepan-4- yl]-2-methyl-6-[5-(1- methylcyclopropoxy)-1H-indazol-3- yl]pyridazin-3-one |
| 93 | 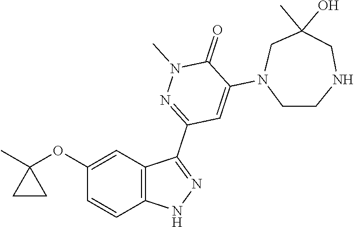 [SMILES: Cn1nc(-c2n[nH]c3ccc(OC4(C)CC4)cc23)cc(N2CCNCC(C)(O)C2)c1=O] | 4-(6-hydroxy-6-methyl-1,4-diazepan-1- yl)-2-methyl-6-[5-(1- methylcyclopropoxy)-1H-indazol-3- yl]pyridazin-3-one |
| 94 | 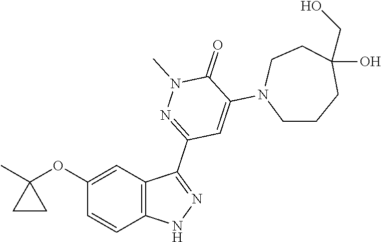 [SMILES: Cn1nc(-c2n[nH]c3ccc(OC4(C)CC4)cc23)cc(N2CCCC(O)(CO)CC2)c1=O] | 4-[4-hydroxy-4-(hydroxymethyl)azepan- 1-yl]-2-methyl-6-[5-(1- methylcyclopropoxy)-1H-indazol-3- yl]pyridazin-3-one |
| 95 | 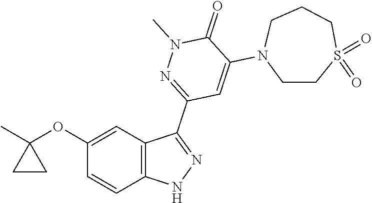 [SMILES: Cn1nc(-c2n[nH]c3ccc(OC4(C)CC4)cc23)cc(N2CCCS(=O)(=O)CC2)c1=O] | 4-(1,1-dioxo-1,4-thiazepan-4-yl)-2- methyl-6-[5-(1-methylcyclopropoxy)- 1H-indazol-3-yl]pyridazin-3-one |
| 96 | 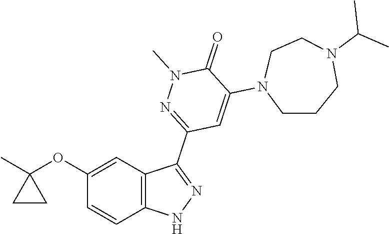 [SMILES: CC(C)N1CCCN(c2cc(-c3n[nH]c4ccc(OC5(C)CC5)cc34)nn(C)c2=O)CC1] | 4-(4-isopropyl-1,4-diazepan-1-yl)-2- methyl-6-[5-(1-methylcyclopropoxy)- 1H-indazol-3-yl]pyridazin-3-one |
| 97 | 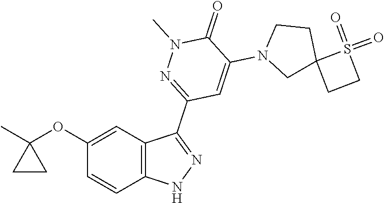 [SMILES: Cn1nc(-c2n[nH]c3ccc(OC4(C)CC4)cc23)cc(N2CCC3(CCS3(=O)=O)C2)c1=O] | 4-(1,1-dioxo-1lambda6-thia-7- azaspiro[3.4]octan-7-yl)-2-methyl-6-[5- (1-methylcyclopropoxy)-1H-indazol-3- yl]pyridazin-3-one |
| 98 | 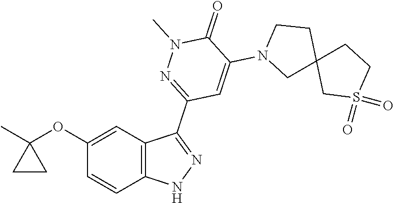 [SMILES: Cn1nc(-c2n[nH]c3ccc(OC4(C)CC4)cc23)cc(N2CCC3(CCS(=O)(=O)C3)C2)c1=O] | 4-(2,2-dioxo-2lambda6-thia-7- azaspiro[4.4]nonan-7-yl)-2-methyl-6-[5- (1-methylcyclopropoxy)-1H-indazol-3- yl]pyridazin-3-one |
| 99 | 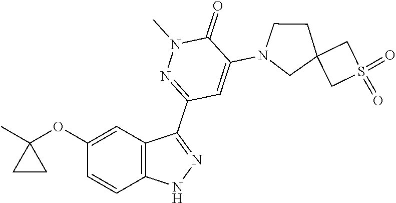 [SMILES: Cn1nc(-c2n[nH]c3ccc(OC4(C)CC4)cc23)cc(N2CCC3(C2)CS(=O)(=O)C3)c1=O] | 4-(2,2-dioxo-2lambda6-thia-7- azaspiro[3.4]octan-7-yl)-2-methyl-6-[5- (1-methylcyclopropoxy)-1H-indazol-3- yl]pyridazin-3-one |
| 100 | 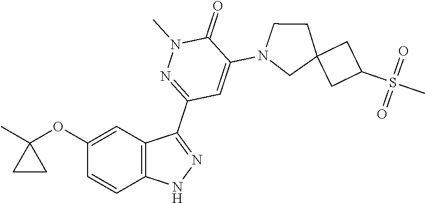 | 2-methyl-6-[5-(1-methylcyclopropoxy)- 1H-indazol-3-yl]-4-(2-methylsulfonyl-6- azaspiro[3.4]octan-6-yl)pyridazin-3-one |
| 101 | 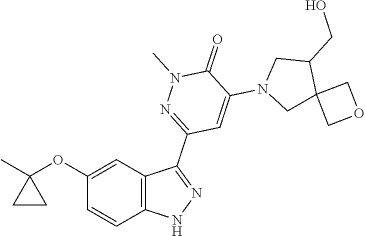 [SMILES: Cn1nc(-c2n[nH]c3ccc(OC4(C)CC4)cc23)cc(N2CC(CO)C3(COC3)C2)c1=O] | 4-[5-(hydroxymethyl)-2-oxa-7- azaspiro[3.4]octan-7-yl]-2-methyl-6-[5- (1-methylcyclopropoxy)-1H-indazol-3- yl]pyridazin-3-one |
| 102 | 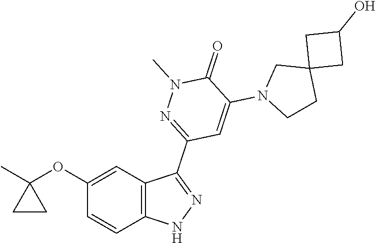 [SMILES: Cn1nc(-c2n[nH]c3ccc(OC4(C)CC4)cc23)cc(N2CCC3(CC(O)C3)C2)c1=O] | 4-(2-hydroxy-6-azaspiro[3.4]octan-6-yl)- 2-methyl-6-[5-(1-methylcyclopropoxy)- 1H-indazol-3-yl]pyridazin-3-one |
| 103 | 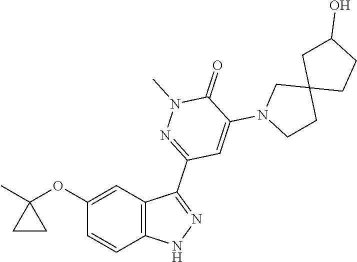 [SMILES: Cn1nc(-c2n[nH]c3ccc(OC4(C)CC4)cc23)cc(N2CCC3(CCC(O)C3)C2)c1=O] | 4-(8-hydroxy-2-azaspiro[4.4]nonan-2- yl)-2-methyl-6-[5-(1- methylcyclopropoxy)-1H-indazol-3- yl]pyridazin-3-one |
| 104 | 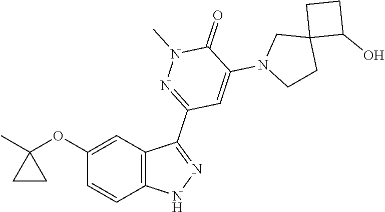 [SMILES: Cn1nc(-c2n[nH]c3ccc(OC4(C)CC4)cc23)cc(N2CCC3(CCC3O)C2)c1=O] | 4-(3-hydroxy-6-azaspiro[3.4]octan-6-yl)- 2-methyl-6-[5-(1-methylcyclopropoxy)- 1H-indazol-3-yl]pyridazin-3-one |
| 105 | 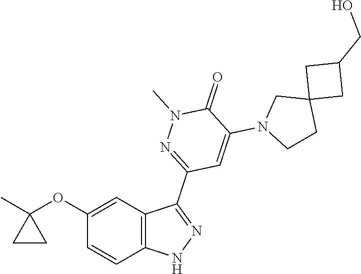 [SMILES: Cn1nc(-c2n[nH]c3ccc(OC4(C)CC4)cc23)cc(N2CCC3(CC(CO)C3)C2)c1=O] | 4-[2-(hydroxymethyl)-6- azaspiro[3.4]octan-6-yl]-2-methyl-6-[5- (1-methylcyclopropoxy)-1H-indazol-3- yl]pyridazin-3-one |
| 106 | 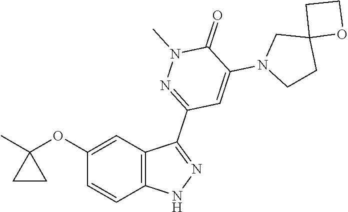 [SMILES: Cn1nc(-c2n[nH]c3ccc(OC4(C)CC4)cc23)cc(N2CCC3(CCO3)C2)c1=O] | 2-methyl-6-[5-(1-methylcyclopropoxy)- 1H-indazol-3-yl]-4-(1-oxa-7- azaspiro[3.4]octan-7-yl)pyridazin-3-one |
| 107 | 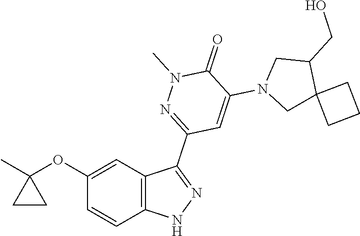 [SMILES: Cn1nc(-c2n[nH]c3ccc(OC4(C)CC4)cc23)cc(N2CC(CO)C3(CCC3)C2)c1=O] | 4-[8-(hydroxymethyl)-6- azaspiro[3.4]octan-6-yl]-2-methyl-6-[5- (1-methylcyclopropoxy)-1H-indazol-3- yl]pyridazin-3-one |
| 108 | 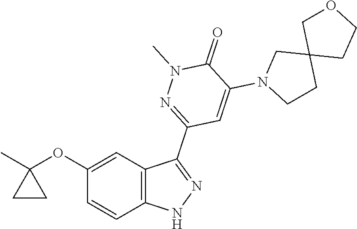 [SMILES: Cn1nc(-c2n[nH]c3ccc(OC4(C)CC4)cc23)cc(N2CCC3(CCOC3)C2)c1=O] | 2-methyl-6-[5-(1-methylcyclopropoxy)- 1H-indazol-3-yl]-4-(2-oxa-7- azaspiro[4.4]nonan-7-yl)pyridazin-3-one |
| 109 | 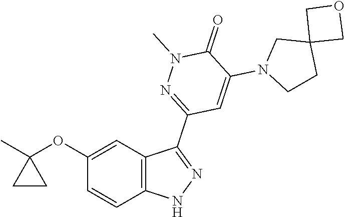 [SMILES: Cn1nc(-c2n[nH]c3ccc(OC4(C)CC4)cc23)cc(N2CCC3(COC3)C2)c1=O] | 2-methyl-6-[5-(1-methylcyclopropoxy)- 1H-indazol-3-yl]-4-(2-oxa-7- azaspiro[3.4]octan-7-yl)pyridazin-3-one |
| 110 | 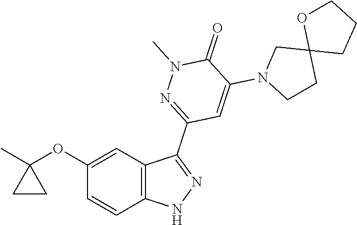 [SMILES: Cn1nc(-c2n[nH]c3ccc(OC4(C)CC4)cc23)cc(N2CCC3(CCCO3)C2)c1=O] | 2-methyl-6-[5-(1-methylcyclopropoxy)- 1H-indazol-3-yl]-4-(1-oxa-7- azaspiro[4.4]nonan-7-yl)pyridazin-3-one |
| 111 | 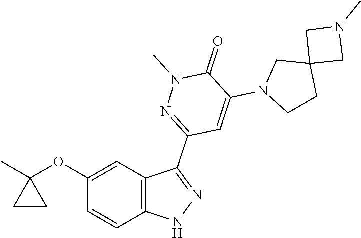 | 2-methyl-6-[5-(1-methylcyclopropoxy)- 1H-indazol-3-yl]-4-(2-methyl-2,6- diazaspiro[3.4]octan-6-yl)pyridazin-3- one |
| 112 | 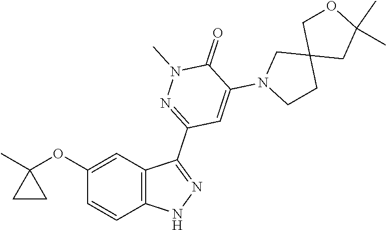 | 4-(3,3-dimethyl-2-oxa-7- azaspiro[4.4]nonan-7-yl)-2-methyl-6-[5- (1-methylcyclopropoxy)-1H-indazol-3- yl]pyridazin-3-one |
| 113 | 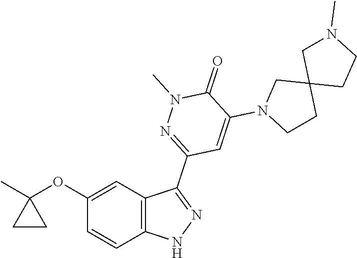 [SMILES: CN1CCC2(CCN(c3cc(-c4n[nH]c5ccc(OC6(C)CC6)cc45)nn(C)c3=O)C2)C1] | 2-methyl-6-[5-(1-methylcyclopropoxy)- 1H-indazol-3-yl]-4-(7-methyl-2,7- diazaspiro[4.4]nonan-2-yl)pyridazin-3- one |
| 114 | 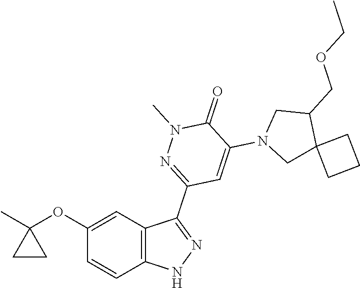 [SMILES: CCOCC1CN(c2cc(-c3n[nH]c4ccc(OC5(C)CC5)cc34)nn(C)c2=O)CC12CCC2] | 4-[8-(ethoxymethyl)-6- azaspiro[3.4]octan-6-yl]-2-methyl-6-[5- (1-methylcyclopropoxy)-1H-indazol-3- yl]pyridazin-3-one |
| 115 | 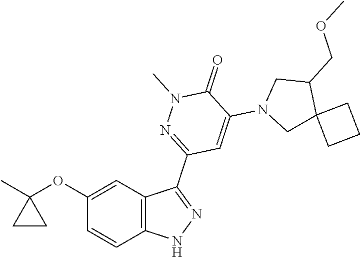 [SMILES: COCC1CN(c2cc(-c3n[nH]c4ccc(OC5(C)CC5)cc34)nn(C)c2=O)CC12CCC2] | 4-[8-(methoxymethyl)-6- azaspiro[3.4]octan-6-yl]-2-methyl-6-[5- (1-methylcyclopropoxy)-1H-indazol-3- yl]pyridazin-3-one |
| 116 | 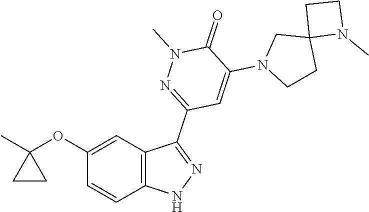 | 2-methyl-6-[5-(1-methylcyclopropoxy)- 1H-indazol-3-yl]-4-(1-methyl-1,7- diazaspiro[3.4]octan-7-yl)pyridazin-3- one |
| 117 | 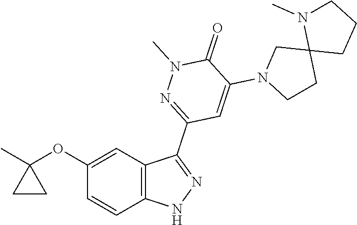 [SMILES: CN1CCCC12CCN(c1cc(-c3n[nH]c4ccc(OC5(C)CC5)cc34)nn(C)c1=O)C2] | 2-methyl-6-[5-(1-methylcyclopropoxy)- 1H-indazol-3-yl]-4-(1-methyl-1,7- diazaspiro[4.4]nonan-7-yl)pyridazin-3- one |
| 118 | 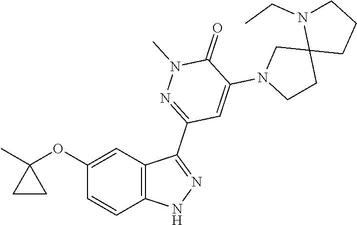 [SMILES: CCN1CCCC12CCN(c1cc(-c3n[nH]c4ccc(OC5(C)CC5)cc34)nn(C)c1=O)C2] | 4-(1-ethyl-1,7-diazaspiro[4.4]nonan-7- yl)-2-methyl-6-[5-(1- methylcyclopropoxy)-1H-indazol-3- yl]pyridazin-3-one |
| 119 | 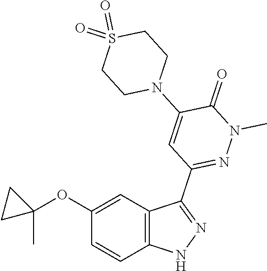 [SMILES: Cn1nc(-c2n[nH]c3ccc(OC4(C)CC4)cc23)cc(N2CCS(=O)(=O)CC2)c1=O] | 4-(1,1-dioxo-1,4-thiazinan-4-yl)-2- methyl-6-[5-(1-methylcyclopropoxy)- 1H-indazol-3-yl]pyridazin-3-one |
| 120 | 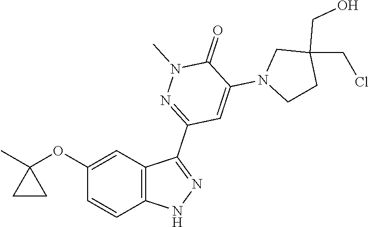 [SMILES: Cn1nc(-c2n[nH]c3ccc(OC4(C)CC4)cc23)cc(N2CCC(CO)(CCl)C2)c1=O] | 4-[3-(chloromethyl)-3- (hydroxymethyl)pyrrolidin-1-yl]-2- methyl-6-[5-(1-methylcyclopropoxy)- 1H-indazol-3-yl]pyridazin-3-one |
| 121 | 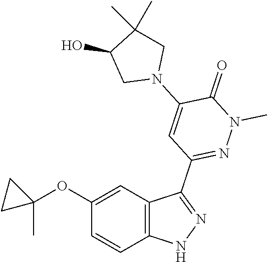 [SMILES: Cn1nc(-c2n[nH]c3ccc(OC4(C)CC4)cc23)cc(N2C[C@@H](O)C(C)(C)C2)c1=O] | 4-[(4R)-4-hydroxy-3,3-dimethyl- pyrrolidin-1-yl]-2-methyl-6-[5-(1- methylcyclopropoxy)-1H-indazol-3- yl]pyridazin-3-one |
| 122 | 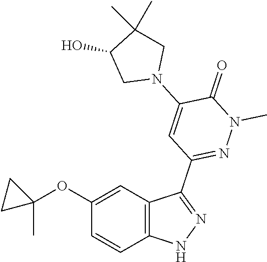 [SMILES: Cn1nc(-c2n[nH]c3ccc(OC4(C)CC4)cc23)cc(N2C[C@H](O)C(C)(C)C2)c1=O] | 4-[(4S)-4-hydroxy-3,3-dimethyl- pyrrolidin-1-yl]-2-methyl-6-[5-(1- methylcyclopropoxy)-1H-indazol-3- yl]pyridazin-3-one |
| 123 | 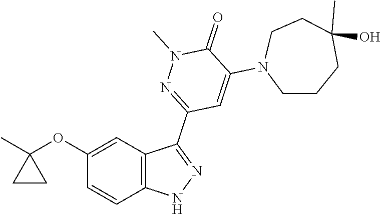 [SMILES: Cn1nc(-c2n[nH]c3ccc(OC4(C)CC4)cc23)cc(N2CCC[C@](C)(O)CC2)c1=O] | 4-[(4S)-4-hydroxy-4-methyl-azepan-1- yl]-2-methyl-6-[5-(1- methylcyclopropoxy)-1H-indazol-3- yl]pyridazin-3-one |
| 124 | 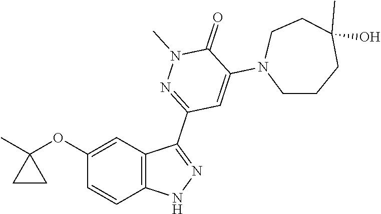 [SMILES: Cn1nc(-c2n[nH]c3ccc(OC4(C)CC4)cc23)cc(N2CCC[C@@](C)(O)CC2)c1=O] | 4-[(4R)-4-hydroxy-4-methyl-azepan-1- yl]-2-methyl-6-[5-(1- methylcyclopropoxy)-1H-indazol-3- yl]pyridazin-3-one |
| 125 | 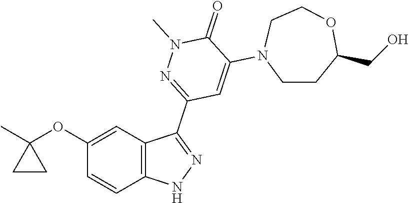 [SMILES: Cn1nc(-c2n[nH]c3ccc(OC4(C)CC4)cc23)cc(N2CCO[C@@H](CO)CC2)c1=O] | 4-[(7R)-7-(hydroxymethyl)-1,4- oxazepan-4-yl]-2-methyl-6-[5-(1- methylcyclopropoxy)-1H-indazol-3- yl]pyridazin-3-one |
| 126 | 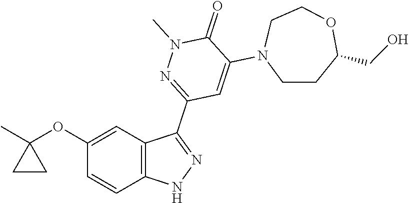 [SMILES: Cn1nc(-c2n[nH]c3ccc(OC4(C)CC4)cc23)cc(N2CCO[C@H](CO)CC2)c1=O] | 4-[(7S)-7-(hydroxymethyl)-1,4- oxazepan-4-yl]-2-methyl-6-[5-(1- methylcyclopropoxy)-1H-indazol-3- yl]pyridazin-3-one |
| 127 | 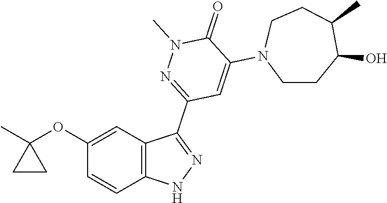 [SMILES: C[C@@H]1CCN(c2cc(-c3n[nH]c4ccc(OC5(C)CC5)cc34)nn(C)c2=O)CC[C@@H]1O] | 4-[(4S,5R)-4-hydroxy-5-methyl-azepan- 1-yl]-2-methyl-6-[5-(1- methylcyclopropoxy)-1H-indazol-3- yl]pyridazin-3-one |
| 128 | 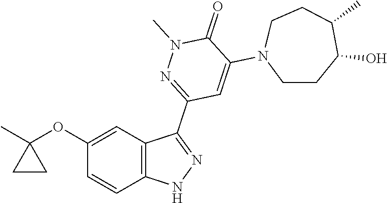 [SMILES: C[C@H]1CCN(c2cc(-c3n[nH]c4ccc(OC5(C)CC5)cc34)nn(C)c2=O)CC[C@H]1O] | 4-[(4R,5S)-4-hydroxy-5-methyl-azepan- 1-yl]-2-methyl-6-[5-(1- methylcyclopropoxy)-1H-indazol-3- yl]pyridazin-3-one |
| 129 | 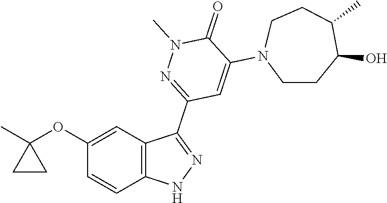 [SMILES: C[C@H]1CCN(c2cc(-c3n[nH]c4ccc(OC5(C)CC5)cc34)nn(C)c2=O)CC[C@@H]1O] | 4-[(4S,5S)-4-hydroxy-5-methyl-azepan- 1-yl]-2-methyl-6-[5-(1- methylcyclopropoxy)-1H-indazol-3- yl]pyridazin-3-one |
| 130 | 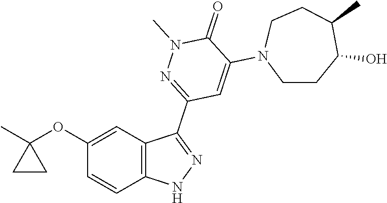 [SMILES: C[C@@H]1CCN(c2cc(-c3n[nH]c4ccc(OC5(C)CC5)cc34)nn(C)c2=O)CC[C@H]1O] | 4-[(4R,5R)-4-hydroxy-5-methyl-azepan- 1-yl]-2-methyl-6-[5-(1- methylcyclopropoxy)-1H-indazol-3- yl]pyridazin-3-one |
| 131 | 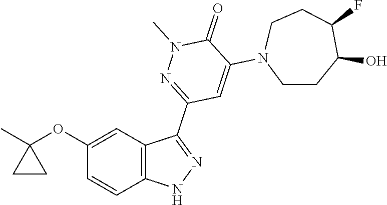 [SMILES: Cn1nc(-c2n[nH]c3ccc(OC4(C)CC4)cc23)cc(N2CC[C@@H](F)[C@@H](O)CC2)c1=O] | 4-[(4R,5S)-4-fluoro-5-hydroxy-azepan-1- yl]-2-methyl-6-[5-(1- methylcyclopropoxy)-1H-indazol-3- yl]pyridazin-3-one |
| 132 | 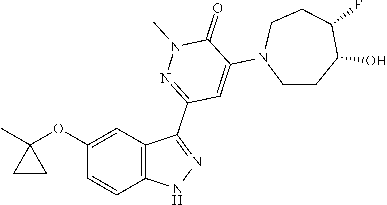 [SMILES: Cn1nc(-c2n[nH]c3ccc(OC4(C)CC4)cc23)cc(N2CC[C@@H](O)[C@@H](F)CC2)c1=O] | 4-[(4S,5R)-4-fluoro-5-hydroxy-azepan-1- yl]-2-methyl-6-[5-(1- methylcyclopropoxy)-1H-indazol-3- yl]pyridazin-3-one |
| 133 | 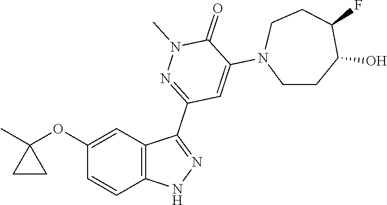 [SMILES: Cn1nc(-c2n[nH]c3ccc(OC4(C)CC4)cc23)cc(N2CC[C@@H](O)[C@H](F)CC2)c1=O] | 4-[(4R,5R)-4-fluoro-5-hydroxy-azepan- 1-yl]-2-methyl-6-[5-(1- methylcyclopropoxy)-1H-indazol-3- yl]pyridazin-3-one |
| 134 | 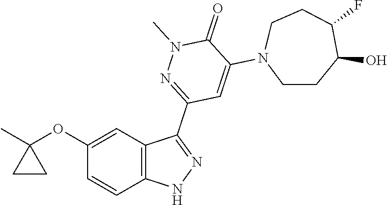 [SMILES: Cn1nc(-c2n[nH]c3ccc(OC4(C)CC4)cc23)cc(N2CC[C@H](O)[C@@H](F)CC2)c1=O] | 4-[(4S,5S)-4-fluoro-5-hydroxy-azepan-1- yl]-2-methyl-6-[5-(1- methylcyclopropoxy)-1H-indazol-3- yl]pyridazin-3-one |

or a pharmaceutically acceptable salt, stereoisomer, solvate, or tautomer thereof.

22. A pharmaceutical composition comprising the compound of claim 1 or a pharmaceutically acceptable salt, stereoisomer, solvate, or tautomer thereof, and a pharmaceutically acceptable carrier.

23. The pharmaceutical composition of claim 22, further comprising one or more additional pharmaceutically active agents.

24. A method of inhibiting of LRRK2 in a cell, comprising contacting the cell with a compound of claim 1.

25. The method of claim 24, wherein the contacting is in vitro or in vivo.

26. A method for the treatment or prevention of a disease or disorder associated with LRRK2 comprising administering to a subject in need thereof a compound of claim 1.

27. The method of claim 26, wherein the disease or disorder is selected from the group consisting of Parkinson Disease 8, Autosomal Dominant (PARK8); Hereditary Late-Onset Parkinson Disease (LOPD); Spinocerebellar Atrophy; Klippel-Feil Syndrome 1, Autosomal Dominant (KFS1); Autosomal Dominant Cerebellar Ataxia (SCA); Parkinson Disease, Late-Onset (PD); Parkinson Disease 2, Autosomal Recessive Juvenile (PARK2); Parkinsonism; Rem Sleep Behavior Disorder; Dementia, Lewy Body (DLB); Lrrk2 Parkinson Disease; Parkinson Disease 3, Autosomal Dominant (PARK3); Early-Onset Parkinson's Disease; Multiple System Atrophy 1 (MSA1); Essential Tremor; Movement Disease; Supranuclear Palsy, Progressive, 1 (PSNP1); Klippel-Feil Syndrome 1; Dementia; Parkinson Disease 10 (PARK10); Tremor; Frontotemporal Dementia (FTD); Postencephalitic Parkinson Disease; Vascular Parkinsonism; Aphasia; Parkinson Disease 1, Autosomal Dominant (PARK1); Athetosis; Klippel-Feil Syndrome (KFS); Kufor-Rakeb Syndrome (KRS); Leprosy 3 (LPRS3); Alzheimer Disease 8 (AD8); Crohn's Disease; Rheumatoid Arthritis (RA); Alzheimer Disease (AD); Color Agnosia; Gaucher Disease, Type I (GD1); Parkinson Disease 15, Autosomal Recessive Early-Onset (PARK15); Von Economo's Disease; Gerstmann-Straussler Disease (GSD); Amyotrophic Lateral Sclerosis-Parkinsonism/dementia Complex 1 (ALS-PDC1); Dystonia; Sphingolipidosis; Radial Nerve Lesion; Toxic Encephalopathy; Sleep Disorder; Pick Disease of Brain (PIDB); Ophthalmomyiasis; Gaucher's Disease (GD); Optic Atrophy 7 with or Without Auditory Neuropathy (OPA7); Ulnar Nerve Lesion; Inflammatory Bowel Disease 4 (IBD4); Inflammatory Bowel Disease; 3-Methylglutaconic Aciduria, Type Iii (MGCA3); Nervous System Disease; Amyotrophic Lateral Sclerosis 1 (ALS1); Mitochondrial Complex I Deficiency, Nuclear Type 1 (MC1DN1).

28. The method of claim 26, wherein the disease or disorder is Parkinson Disease (PD).

29. The method of any one of claims 24-28, wherein the subject is a mammal.

30. The method of claim 29, wherein the subject is a human.
